(19) United States
(12) Patent Application Publication
(10) Pub. No.: US 2020/0247800 A1
(43) Pub. Date: Aug. 6, 2020

(54) SUBSTITUTED BICYCLIC HETEROCYCLIC COMPOUNDS AS NADPH OXIDASE INHIBITORS

(71) Applicant: Glenmark Pharmaceuticals S.A., La Chaux-de-Fonds (CH)

(72) Inventors: Sukeerthi Kumar, Navi Mumbai, Maharashtra (IN); Abraham Thomas, Navi Mumbai, Maharashtra (IN); Sachin Sundarlal Chaudhari, Navi Mumbai, Maharshtra (IN); Laxmikant Atmaram Gharat, Thane (W), Maharashtra (IN); Neelima Khairatkar-Joshi, Thane (W), Maharashtra (IN); Daisy Manish Shah, Mumbai (IN); Indranil Mukhopadhyay, Navi Mumbai, Maharashtra (IN)

(21) Appl. No.: 16/610,351

(22) PCT Filed: May 4, 2018
(86) PCT No.: PCT/IB2018/053121

§ 371 Date: Nov. 1, 2019

(30) Foreign Application Priority Data
May 4, 2017 (IN) 201721015787

(51) Int. Cl. C07D 471/04

(52) U.S. Cl. CPC C07D 471/04

(57) ABSTRACT

The present application relates to substituted fused heteroaryl and hetero-cyclic compounds, useful as nicotinamide adenine dinucleotide phosphate oxidase inhibitors (NADPH oxidase inhibitors), processes for their preparation, pharmaceutical compositions comprising the compounds, and the use of the compounds or the compositions in the treatment or prevention of various diseases, conditions and/or disorders mediated by NADPH oxidase. (Formula I)

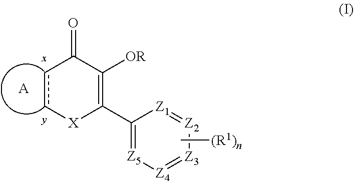

### RELATED APPLICATIONS

This application claims the benefit of Indian Provisional Application No. 201721015787 filed on May 4, 2017; which is hereby incorporated by reference in its entirety.

### TECHNICAL FIELD OF THE INVENTION

The present application relates to substituted fused heteroaryl and heterocyclic compounds, useful as nicotinamide adenine dinucleotide phosphate oxidase inhibitors (NADPH oxidase inhibitors), processes for their preparation, pharmaceutical compositions comprising the compounds, and the use of the compounds or the compositions in the treatment or prevention of various diseases, conditions and/or disorders mediated by NADPH oxidase.

### BACKGROUND OF THE INVENTION

The NOX family NADPH oxidases (nicotinamide adenine dinucleotide phosphate oxidase) comprise a family of reactive oxygen species (ROS)-producing enzymes that is increasingly recognized as a source of oxidative stress in many disease settings. Whereas NOX2 (also known as gp91phox), the phagocyte oxidase, has been known for several decades as the enzyme responsible for the oxidative burst and associated microbicidal activity, the other members of the gene family have been identified only recently. The NOX family now consists of seven members (NOX1, NOX2, NOX3, NOX4, NOX5, DUOX1, and DUOX2), each with a distinct tissue distribution. Since the discovery that NOX enzymes are not limited to white blood cells, an exponential increase in scientific reports describe how NOX enzymes are responsible for increased ROS generation in numerous pathologic conditions, such as inflammation, hypertension, ischemia/reperfusion, diabetes, cardiovascular diseases and neuro-degeneration (Lambeth et al., Semin Immunopathol 30: 339-363, 2008). The elevated ROS production has been linked to the pathobiology of many of these conditions (Lambeth et al., Semin Immunopathol 30: 339-363, 2008).

NADPH oxidase generates superoxide by transferring electrons from NADPH inside the cell across the membrane and coupling these to molecular oxygen to produce superoxide anion, a reactive free-radical. Superoxide can be produced in phagosomes, which contain ingested bacteria and fungi, or it can be produced outside of the cell. In a phagosome, superoxide can spontaneously form hydrogen peroxide that will undergo further reactions to generate reactive oxygen species (ROS).

Reactive oxygen species are oxygen-derived small molecules, including oxygen radicals [superoxide (O2.⁻), hydroxyl (.OH), peroxyl (RO₂.), and alkoxyl (RO.)]and certain non-radicals that are either oxidizing agents and/or are easily converted into radicals, such as hypochlorous acid (HOCl), ozone (O3), singlet oxygen (1O2), and hydrogen peroxide (H₂O2). Nitrogen-containing oxidants, such as nitric oxide, are called reactive nitrogen species (RNS). ROS generation is generally a cascade of reactions that starts with the production of superoxide. Superoxide rapidly dismutates to hydrogen peroxide either spontaneously, particularly at low pH or catalyzed by superoxide dismutase. Other elements in the cascade of ROS generation include the reaction of superoxide with nitric oxide to form peroxynitrite, the peroxidase-catalyzed formation of hypochlorous acid from hydrogen peroxide, and the iron-catalyzed Fenton reaction leading to the generation of hydroxyl radical (Klebanoff et al., Ann Intern Med, 1980, 93: 480-489; Thannickal et al., Am J Physiol Lung Cell Mol Physiol, 2000, 279: L1005-L1028). ROS avidly interact with a large number of molecules including other small inorganic molecules as well as DNA, proteins, lipids, carbohydrates and nucleic acids. This initial reaction may generate a second radical, thus multiplying the potential damage. ROS are involved not only in cellular damage and killing of pathogens, but also in a large number of reversible regulatory processes in virtually all cells and tissues. However, despite the importance of ROS in the regulation of fundamental physiological processes, ROS production can also irreversibly destroy or alter the function of the target molecule. Consequently, ROS have been increasingly identified as major contributors to damage in biological organisms, so-called “oxidative stress”.

During inflammation, NADPH oxidase is one of the most important sources of ROS production in vascular cells under inflammatory conditions (Thabut et al, J. Biol. Chem., 2002, 277:22814-22821). In the lungs, tissues are constantly exposed to oxidants that are generated either endogenously by metabolic reactions (e.g. by mitochondrial respiration or activation of recruited inflammatory cells) or exogenously in the air (e.g. cigarette smoke or air pollutants). Further, the lungs, constantly exposed to high oxygen tensions as compared to other tissues, have a considerable surface area and blood supply and are particularly susceptible to injury mediated by ROS (Brigham, Chest, 1986, 89(6): 859-863). NADPH oxidase-dependent ROS generation has been described in pulmonary endothelial cells and smooth muscle cells. NADPH oxidase activation in response to stimuli has been thought to be involved in the development of respiratory disorders such as pulmonary hypertension and enhancement of pulmonary vasoconstriction (Djordjevic et al, Arterioscler. Thromb. Vase. Biol, 2005, 25, 519-525; Liva et al, Am. J. Physiol. Lung, Cell. Mol. Physiol, 2004, 287: L111-118). Further, pulmonary fibrosis has been characterized by lung inflammation and excessive generation of ROS. Osteoclasts, which are macrophage-like cells that play a crucial role in bone turn-over (e.g. bone resorption), generate ROS through NADPH oxidase-dependent mechanisms (Yang et al, J. Cell. Chem., 2002, 84, 645-654). Diabetes is known to increase oxidative stress (e.g. increased generation of ROS by auto-oxidation of glucose) both in humans and animals and increased oxidative stress has been said to play an important role in the development of diabetic complications. It has been shown that increased peroxide localization and endothelial cell dysfunction in the central retina of diabetic rats coincides with the areas of NADPH oxidase activity in the retinal endothelial cells (Ellis et al, Free Rad. Biol. Med., 2000, 28:91-101). Further, it has been suggested that controlling oxidative stress (ROS) in mitochondria and/or inflammation may be a beneficial approach for the treatment of diabetes (Pillarisetti et al., Expert Opin. Ther. Targets, 2004, 8(5):401-408).

ROS are also strongly implicated in the pathogenesis of atherosclerosis, cell proliferation, hypertension and reperfusion injury cardiovascular diseases in general (Cai et al., Trends Pharmacol. ScL, 2003, 24:471-478). Not only is superoxide production, for example in the arterial wall, increased by all risk factors for atherosclerosis, but ROS also induce many “proatherogenic” in vitro cellular responses. The increase in NADPH oxidase activity in vascular wall after balloon injury has been reported (Shi et al, 2001, Throm. Vase. Biol, 2001, 21, 739-745).

It is believed that oxidative stress or free radical damage is also a major causative factor in neurodegenerative diseases. Such damages may include mitochondrial abnormalities, neuronal demyelination, apoptosis, neuronal death and reduced cognitive performance potentially leading to the development of progressive neurodegenerative disorders (Nunomura et al, J. Neuropathol Exp. Neurol, 2001, 60:759-767; Girouard, J. Appl Physiol., 2006, 100:328-335).

Thus, ROS derived from NADPH oxidase contribute to the pathogenesis of numerous diseases, especially cardiovascular diseases or disorders, respiratory disorder or disease, disease or disorder affecting the metabolism, bone disorders, neurodegenerative diseases, inflammatory diseases, reproduction disorder or disease, pain, cancer and disease or disorders of the gastrointestinal system. Therefore, it would be highly desirable to develop new active agents focusing on the ROS signalling cascade, especially on NADPH oxidases (NOX).

Several patent applications relate to various scaffolds and compounds useful as NADPH oxidase inhibitors. PCT publication numbers WO2008113856, WO2010035217, WO2010035219, WO2010035220 and WO2010035221 disclose pyrazolo pyridine derivatives as NADPH oxidase inhibitors in the treatment of cardiovascular diseases, respiratory disorders and disorders affecting the metabolism, skin and/or bone diseases, neurodegenerative diseases, kidney diseases, reproduction disorders, inflammatory disorders and cancer.

The object of the present invention is also to provide compounds as nicotinamide adenine dinucleotide oxidase inhibitors (NADPH oxidase inhibitors) and a method for use of such compounds in treating or ameliorating a disease or disorder wherein inhibition of NADPH oxidase is required.

### SUMMARY OF THE INVENTION

In one aspect, the present invention relates to compound of formula (I)

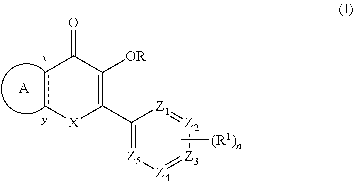

or a pharmaceutically acceptable salt thereof,
wherein,

dotted line [- - - -] inside the ring represents an optional single bond;

X is NH or O;

R is selected from hydrogen, C₁₋₈alkyl and —C(O)R⁷;

Z₁ is CH or S;

Z₂ is CH;

Z₃ is CH or N;

Z₄ is CH;

Z₅ is CH or absent;

ring A is selected from

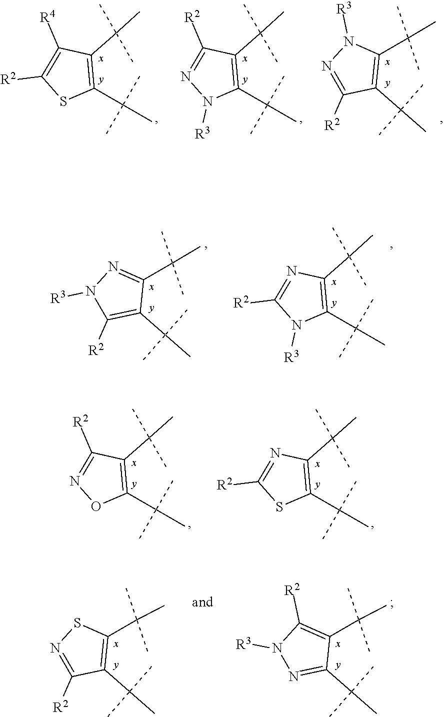

wherein x and y represents the point of attachment;

at each occurrence, R¹ is independently selected from halogen, amino, hydroxyl, C₁₋₈alkyl, C₁₋₈alkoxy, C₁₋₈alkoxyC₁₋₈alkoxy, haloC₁₋₈alkyl, haloC₁₋₈alkoxy, —(CH₂)ₘNR⁵C(O)R⁶, —(CH₂)ₘOR⁵, —(CH₂)ₘNR⁷S(O)_{P}R⁸, C₆₋₁₄aryl and 5- to 14-membered heteroaryl; wherein C₆₋₁₄aryl is optionally substituted with one or more substituents selected from halogen and C₁₋₈alkyl;

at each occurrence, R² is independently selected from hydrogen, C₁₋₈alkyl, haloC₁₋₈alkyl, hydroxyC₁₋₈alkyl, —(CH₂)ₘNR⁵C(O)NR⁶, —(CH₂)ₘOR⁵, 3- to 15-membered heterocyclyl, 3- to 15-membered heterocyclylC₁₋₈alkyl, C₆₋₁₄aryl and C₆₋₁₄arylC₁₋₈alkyl; wherein 3- to 15-membered heterocyclyl, 3- to 15-membered heterocyclylC₁₋₈alkyl, C₆₋₁₄aryl and C₆₋₁₄arylC₁₋₈alkyl are optionally substituted with one or more substituents selected from halogen, C₁₋₈alkyl, haloC₁₋₈alkyl, —(CH₂)ₘS(O)_{P}R⁸, C₃₋₁₂cycloalkyl and 3- to 15-membered heterocyclyl;

at each occurrence, R³ is independently selected from hydrogen, C₁₋₈alkyl, haloC₁₋₈alkyl, hydroxyC₁₋₈alkyl, —(CH₂)ₘOR⁵, —(CH)₂N(R⁵)₂, —(CH₂)ₘS(O)_{P}R⁸, C₃₋₁₂cycloalkyl, 3- to 15-membered heterocyclyl, 3- to 15-membered heterocyclylC₁₋₈alkyl, C₆₋₁₄aryl, C₆₋₁₄arylC₁₋₈alkyl, 5- to 14-membered heteroaryl and 5- to 14-membered heteroarylC₁₋₈alkyl; wherein C₃₋₁₂cycloalkyl, 3- to 15-membered heterocyclylC₁₋₈alkyl, C₆₋₁₄aryl and C₆₋₁₄arylC₁₋₈alkyl are optionally substituted with one or more substituents selected from halogen, oxo, C₁₋₈alkyl and C₁₋₈alkoxy;

at each occurrence, R⁴ is independently selected from hydrogen and C₁₋₈alkyl;

at each occurrence, R⁵ is independently selected from hydrogen and C₁₋₈alkyl;

at each occurrence, R⁶ is independently selected from hydrogen and C₁₋₈alkyl;

at each occurrence, R⁷ is independently selected from hydrogen and C₁₋₈alkyl;

at each occurrence, R⁸ is independently selected from hydrogen and C₁₋₈alkyl;

‘m’ is an integer ranging from 0 to 4, both inclusive;

‘n’ is an integer ranging from 0 to 5, both inclusive; and

‘p’ is an integer ranging from 0 to 2, both inclusive.

The compounds of formula (I) may involve one or more embodiments. Embodiments of formula (I) include compounds of formula (II), as described hereinafter. It is to be understood that the embodiments below are illustrative of the present invention and are not intended to limit the claims to the specific embodiments exemplified. It is also to be understood that the embodiments defined herein may be used independently or in conjunction with any definition, any other embodiment defined herein. Thus the invention contemplates all possible combinations and permutations of the various independently described embodiments. For example, the invention provides compounds of formula (I) as defined above wherein R is hydrogen, methyl or —C(O)CH₃ (according to an embodiment defined below), X is NH or O (according to another embodiment defined below), R¹ is F, Cl, NH₂, OH, methyl, methoxy, —OCH₂CH₂OCH₃, CF₃, OCF₃,

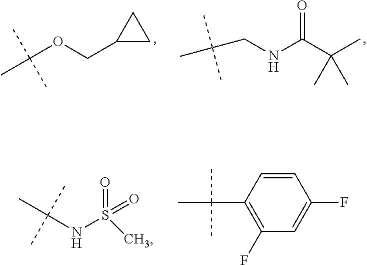

or 1H-imidazol-1-yl (according to yet another embodiment defined below).

According to one embodiment, specifically provided are compounds of formula (I), in which R is hydrogen, C₁₋₈alkyl or —C(O)R⁷.

According to another embodiment, specifically provided are compounds of formula (I), in which R is hydrogen, C₁₋₈alkyl (e.g. methyl) or —C(O)R⁷. In this embodiment, R⁷ is C₁₋₈alkyl (e.g. methyl).

According to yet another embodiment, specifically provided are compounds of formula (I), in which R is hydrogen, methyl or —C(O)R⁷. In this embodiment, R⁷ is methyl.

According to yet another embodiment, specifically provided are compounds of formula (I), in which R is hydrogen, methyl or —C(O)CH₃.

According to another embodiment, specifically provided are compounds of formula (I), in which R is hydrogen.

According to yet another embodiment, specifically provided are compounds of formula (I), in which R is C₁₋₈alkyl (e.g. methyl).

According to yet another embodiment, specifically provided are compounds of formula (I), in which R is methyl.

According to yet another embodiment, specifically provided are compounds of formula (I), in which R is —C(O)R⁷. In this embodiment R⁷ is C₁₋₈alkyl (e.g. methyl).

According to yet another embodiment, specifically provided are compounds of formula (I), in which R is —C(O)R⁷. In this embodiment R⁷ is methyl.

According to yet another embodiment, specifically provided are compounds of formula (I), in which R is —C(O)CH₃.

According to yet another embodiment, specifically provided are compounds of formula (I), in which X is NH or O.

According to yet another embodiment, specifically provided are compounds of formula (I), in which X is NH.

According to yet another embodiment, specifically provided are compounds of formula (I), in which X is O.

According to yet another embodiment, specifically provided are compounds of formula (I), in which Z₁ is CH or S, Z₂ is CH, Z₃ is CH or N, Z₄ is CH and Z₅ is CH or absent.

According to yet another embodiment, specifically provided are compounds of formula (I), in which Z₁ is CH, Z₂ is CH, Z₃ is CH or N, Z₄ is CH and Z₅ is CH.

According to yet another embodiment, specifically provided are compounds of formula (I), in which Z₁ is CH, Z₂ is CH, Z₃ is CH, Z₄ is CH and Z₅ is CH.

According to yet another embodiment, specifically provided are compounds of formula (I), in which Z₁ is CH, Z₂ is CH, Z₃ is N, Z₄ is CH and Z₅ is CH.

According to yet another embodiment, specifically provided are compounds of formula (I), in which Z₁ is S, Z₂ is CH, Z₃ is N, Z₄ is CH and Z₅ is absent.

According to yet another embodiment, specifically provided are compounds of formula (I), in which Z₁ is CH.

According to yet another embodiment, specifically provided are compounds of formula (I), in which Z₁ is S.

According to yet another embodiment, specifically provided are compounds of formula (I), in which Z₃ is CH.

According to yet another embodiment, specifically provided are compounds of formula (I), in which Z₅ is CH.

According to yet another embodiment, specifically provided are compounds of formula (I), in which Z₃ is N.

According to yet another embodiment, specifically provided are compounds of formula (I), in which Z₅ is absent.

According to yet another embodiment, specifically provided are compounds of formula (I), in which R¹ is halogen (e.g. F, Cl, or Br), amino (e.g. NH₂), hydroxyl (e.g. OH), C₁₋₈alkyl (e.g. methyl), C₁₋₈alkoxy (e.g methoxy), C₁₋₈alkoxyC₁₋₈alkoxy (e.g. —OCH₂CH₂OCH₃), haloC₁₋₈alkyl (e.g. CF₃), haloC₁₋₈alkoxy (e.g OCF₃), —(CH₂)ₘOR⁵

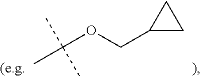

—(CH₂)ₘNR⁵C(O)R⁶

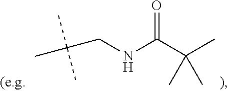

—(CH₂)ₘNR⁷S(O)_{P}R⁸

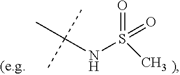

C₆₋₁₄aryl (e.g. phenyl) optionally substituted with one or more substituents selected from halogen (e.g. Cl, F or Br) and 5- to 14-membered heteroaryl (e.g. 1H-imidazol-1-yl). In this embodiment, R⁵ is hydrogen or C₃₋₁₂cycloalkylC₁₋₈alkyl

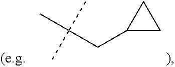

R⁶ is C₁₋₈alkyl

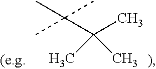

R⁷ is hydrogen, R⁸ is C₁₋₈alkyl (e.g. methyl), ‘p’ is 2 and ‘m’ is 0 or 1.

According to yet another embodiment, specifically provided are compounds of formula (I), in which R¹ is halogen (e.g. F, Cl, or Br), amino (e.g. NH₂), hydroxyl (e.g. OH), C₁₋₈alkyl (e.g. methyl), C₁₋₈alkoxy (e.g methoxy), C₁₋₈alkoxyC₁₋₈alkoxy (e.g. —OCH₂CH₂OCH₃), haloC₁₋₈alkyl (e.g. CF₃), haloC₁₋₈alkoxy (e.g OCF₃), —(CH₂)ₘOR⁵

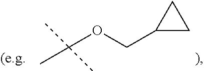

(CH₂)ₘNR⁵C(O)R⁶

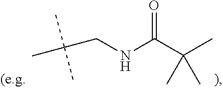

—(CH₂)ₘNR⁷S(O)_{P}R⁸

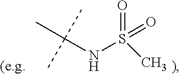

### C₆₋₁₄aryl

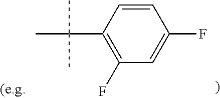

or 5- to 14-membered heteroaryl (e.g. 1H-imidazol-1-yl). In this embodiment, R⁵ is hydrogen or

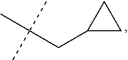

### R⁶ is

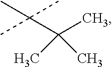

R⁷ is hydrogen, R⁸ is methyl, ‘p’ is 2 and ‘m’ is 0 or 1.

According to yet another embodiment, specifically provided are compounds of formula (I), in which R¹ is F, Cl, NH₂, OH, methyl, methoxy, —OCH₂CH₂OCH₃, CF₃, OCF₃,

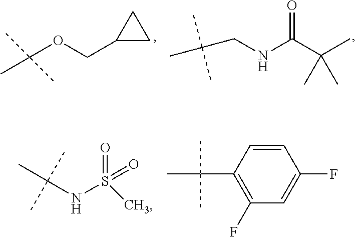

### or 1H-imidazol-1-yl.

According to yet another embodiment, specifically provided are compounds of formula (I), in which ‘n’ is 0, 1, 2 or 3.

According to yet another embodiment, specifically provided are compounds of formula (I), in which ‘n’ is 0.

According to yet another embodiment, specifically provided are compounds of formula (I), in which ‘n’ is 1.

According to yet another embodiment, specifically provided are compounds of formula (I), in which ‘n’ is 2.

According to yet another embodiment, specifically provided are compounds of formula (I), in which ‘n’ is 3.

According to yet another embodiment, specifically provided are compounds of formula (I), in which R¹ is F, Cl, NH₂, OH, methyl, methoxy, —OCH₂CH₂OCH₃, CF₃, OCF₃,

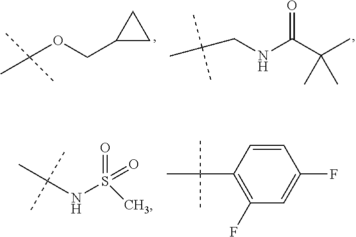

or 1H-imidazol-1-yl and ‘n’ is 0, 1, 2 or 3.

According to yet another embodiment, specifically provided are compounds of formula (I), in which ring A is

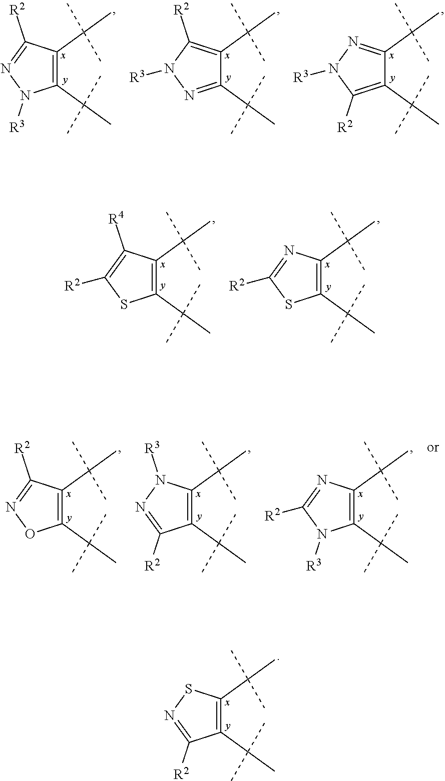

According to yet another embodiment, specifically provided are compounds of formula (I), in which R² is hydrogen, C₁₋₈alkyl (e.g. methyl, ethyl, isopropyl or isobutyl), haloC₁₋₈alkyl (e.g. trifluoromethyl or difluoromethyl), hydroxyC₁₋₈alkyl (e.g. —CH₂OH), —(CH₂)ₘOR⁵ (e.g. —CH₂OCH₃), —(CH₂)ₘC(O)NR⁵R⁶ (e.g. —C(O)NH₂), 3- to 15-membered heterocyclyl

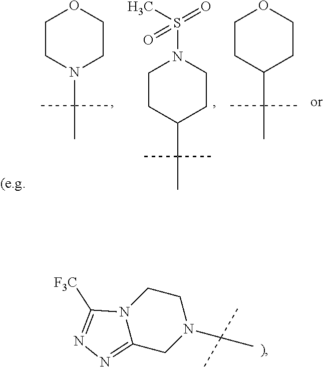

3- to 15-membered heterocyclylC₁₋₈alkyl

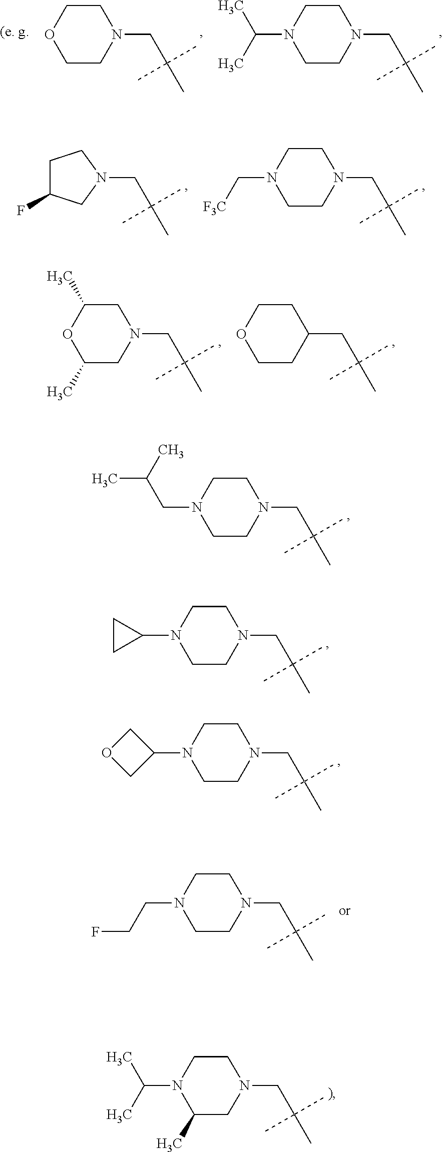

### C₆₋₁₄aryl

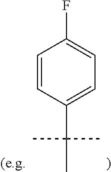

or C₆₋₁₄arylC₁₋₈alkyl

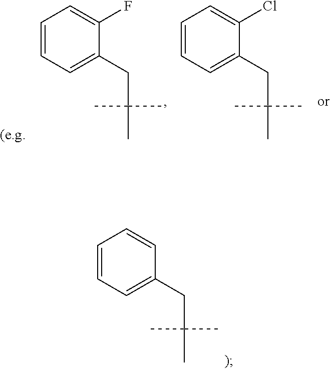

wherein 3- to 15-membered heterocyclyl, 3- to 15-membered heterocyclylC₁₋₈alkyl, C₆₋₁₄aryl and C₆₋₁₄ arylC₁₋₈alkyl are optionally substituted with one or more substituents selected from halogen (e.g. Cl, F or Br), C₁₋₈alkyl (e.g. methyl, 2-methylpropyl or prop-2-yl), haloC₁₋₈alkyl (e.g. trifluoromethyl, trifluoroethyl or fluoroethyl), —(CH₂)ₘS(O)_{P}R⁸ (e.g. —S(O)₂CH₃), C₃₋₁₂cycloalkyl (e.g. cyclopropyl) and 3- to 15-membered heterocyclyl (e.g. oxatane). In this embodiment, R⁵ is hydrogen or C₁₋₈alkyl (e.g. methyl); R⁶ is hydrogen; ‘p’ is 2 and ‘m’ is 0 or 1.

According to yet another embodiment, specifically provided are compounds of formula (I), in which R² is hydrogen, C₁₋₈alkyl (e.g. methyl, ethyl, isopropyl or isobutyl), haloC₁₋₈alkyl (e.g. trifluoromethyl or difluoromethyl), hydroxyC₁₋₈alkyl (e.g. —CH₂OH), —(CH₂)ₘOR⁵ (e.g. —CH₂OCH₃), —(CH₂)ₘC(O)NR⁵R⁶ (e.g. —C(O)NH₂), 3- to 15-membered heterocyclyl

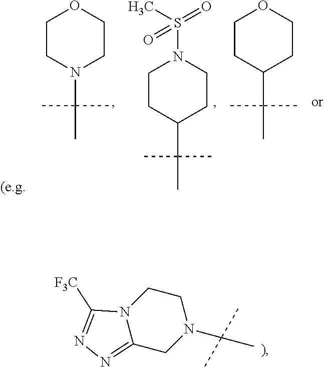

3- to 15-membered heterocyclylC₁₋₈alkyl

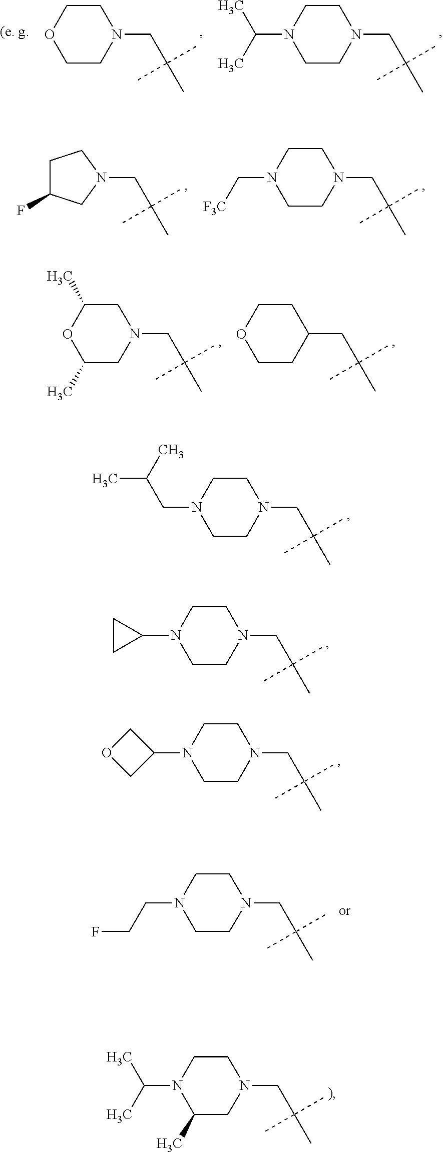

C₆₋₁₄ aryl

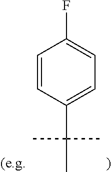

or C₆₋₁₄arylC₁₋₈alkyl

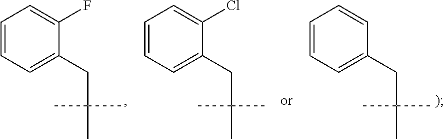

wherein 3- to 15-membered heterocyclyl, 3- to 15-membered heterocyclylC₁₋₈alkyl, C₆₋₁₄aryl and C₆₋₁₄ arylC₁₋₈alkyl are optionally substituted with one or more substituents selected from Cl, F, methyl, 2-methylpropyl, trifluoromethyl, trifluoroethyl, fluoroethyl, —S(O)₂CH₃), cyclopropyl and oxatane. In this embodiment, R⁵ is hydrogen or methyl; R⁶ is hydrogen; ‘p’ is 2 and ‘m’ is 0 or 1.

According to yet another embodiment, specifically provided are compounds of formula (I), in which R² is hydrogen, methyl, ethyl, isopropyl, isobutyl, trifluoromethyl, difluoromethyl, —CH₂OH, —CH₂OCH₃, —C(O)NH₂),

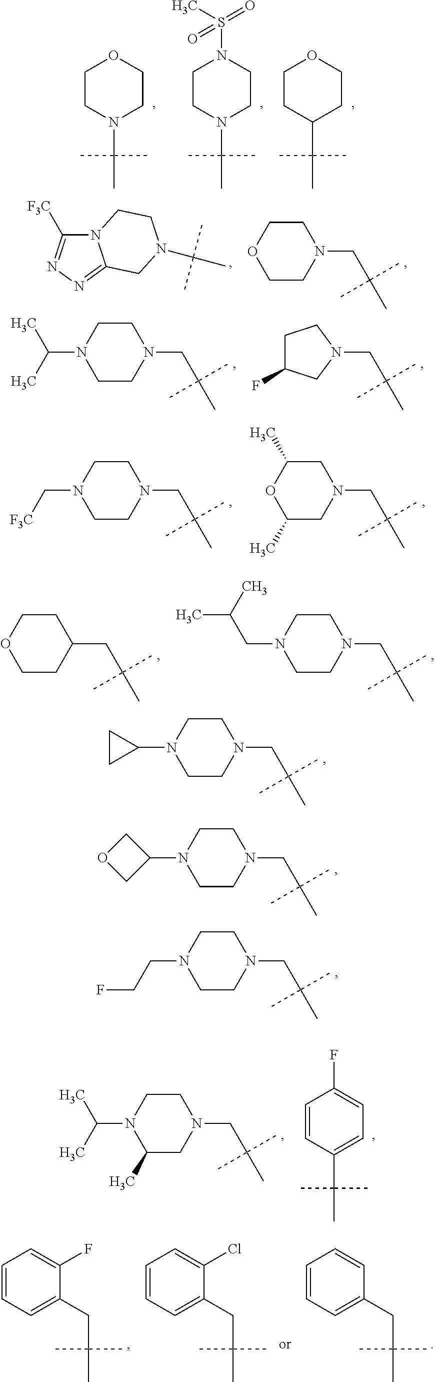

According to yet another embodiment, specifically provided are compounds of formula (I), in which R³ is hydrogen, C₁₋₈alkyl (e.g. methyl, ethyl, propyl, isopropyl, isobutyl or isopentyl), haloC₁₋₈alkyl (e.g. trifluoroethyl), —(CH₂)ₘOR⁵ (e.g. —CH₂CH₂OCH₃ or —CH₂CH₂CH₂OCH₃), hydroxyC₁₋₈alkyl (e.g. —CH₂CH₂OH or —CH₂CH₂CH₂OH), —(CH)₂N(R⁵)₂ (e.g. —CH₂CH₂N(CH₃)₂), C₃₋₁₂cycloalkyl

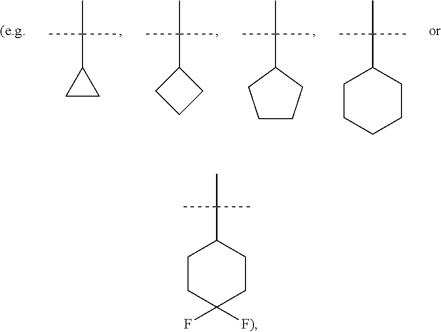

3- to 15-membered heterocyclyl

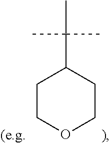

3- to 15-membered heterocyclylC₁₋₈alkyl

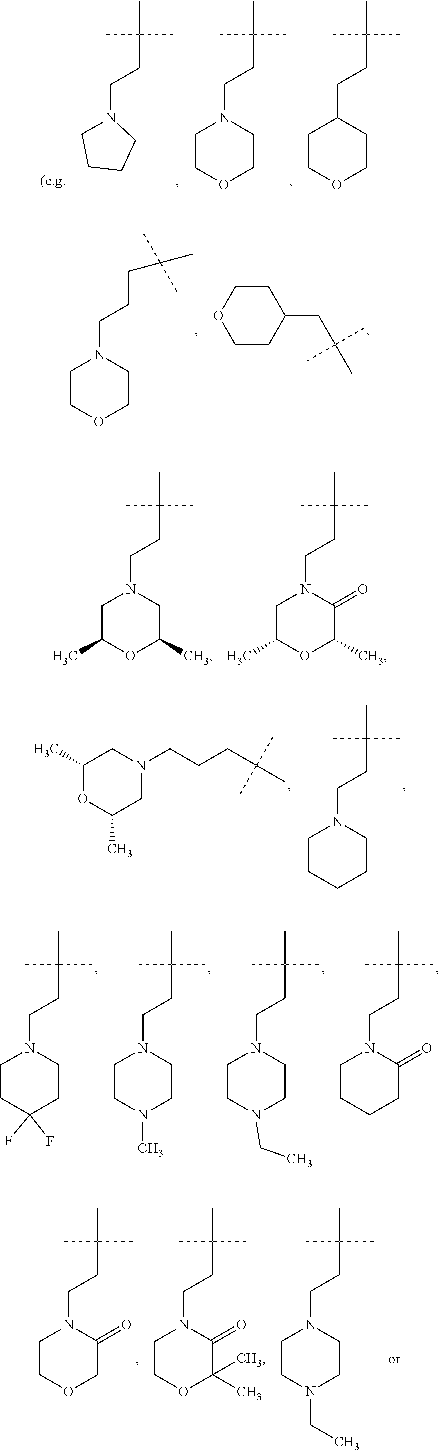

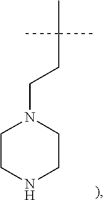

### C₆₋₁₄aryl

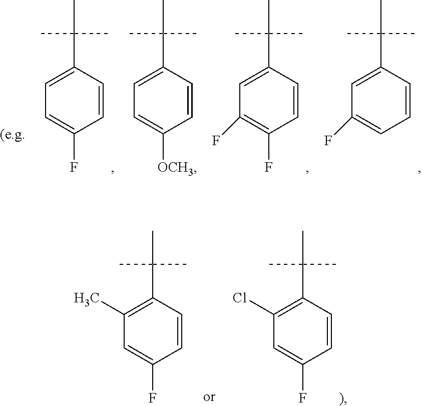

C₆₋₁₄arylC₁₋₈alkyl

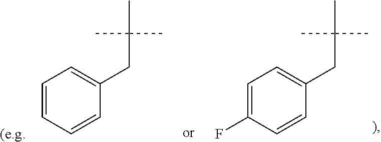

5- to 14-membered heteroaryl

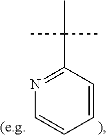

5- to 14-membered heteroarylC₁₋₈alkyl

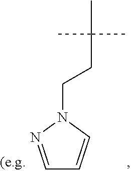

or —(CH₂)ₘS(O)_{P}R⁸

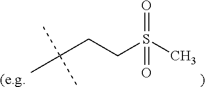

wherein C₃₋₁₂cycloalkyl, 3- to 15-membered heterocyclylC₁₋₈alkyl, C₆₋₁₄aryl and C₆₋₁₄arylC₁₋₈alkyl are optionally substituted with one or more substituents selected from halogen (e.g. Cl, F or Br), oxo (e.g. ═O), C₁₋₈alkyl (e.g. methyl or ethyl) and C₁₋₈alkoxy (e.g. methoxy). In this embodiment, R⁵ is C₁₋₈alkyl (e.g methyl); R⁸ is C₁₋₈alkyl (e.g methyl); m′ is 2 or 3 and ‘p’ is 2.

According to yet another embodiment, specifically provided are compounds of formula (I), in which R³ is hydrogen, C₁₋₈alkyl (e.g. methyl, ethyl, propyl, isopropyl, isobutyl or isopentyl), haloC₁₋₈alkyl (e.g. trifluoroethyl), —(CH₂)ₘOR⁵ (e.g. —CH₂CH₂OCH₃ or —CH₂CH₂CH₂OCH₃), hydroxyC₁₋₈alkyl (e.g. —CH₂CH₂OH or —CH₂CH₂CH₂OH), —(CH)₂N(R⁵)₂ (e.g. —CH₂CH₂N(CH₃)₂), C₃₋₁₂cycloalkyl

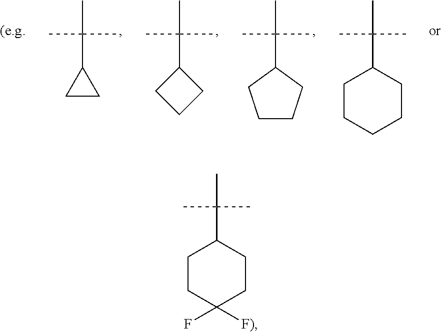

3- to 15-membered heterocyclyl

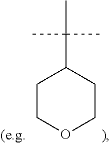

3- to 15-membered heterocyclylC₁₋₈alkyl

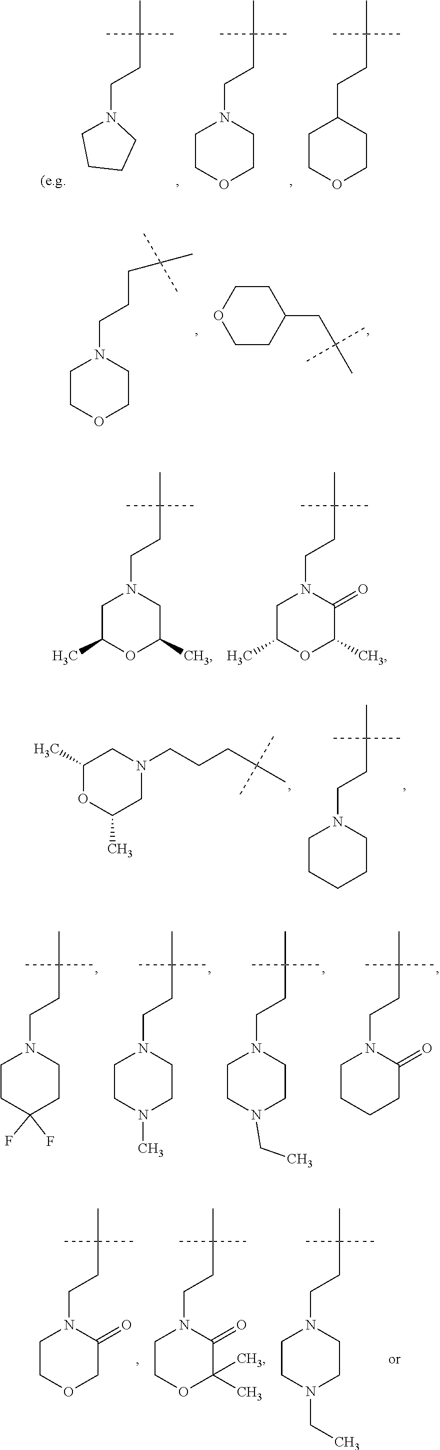

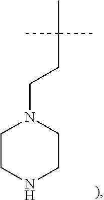

### C₆₋₁₄aryl

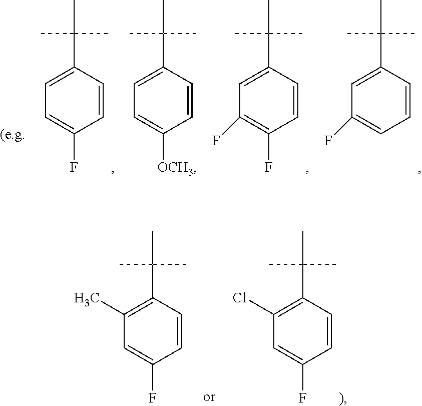

C₆₋₁₄arylC₁₋₈alkyl

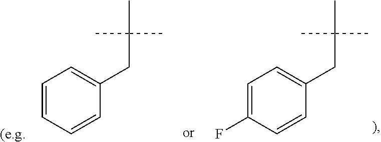

5- to 14-membered heteroaryl

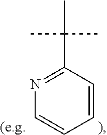

5- to 14-membered heteroarylC₁₋₈alkyl

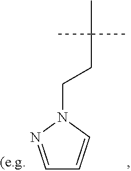

or —(CH₂)ₘS(O)_{P}R⁸

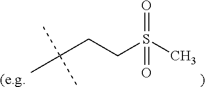

wherein C₃₋₁₂cycloalkyl, 3- to 15-membered heterocyclylC₁₋₈alkyl, C₆₋₁₄aryl and C₆₋₁₄arylC₁₋₈alkyl are optionally substituted with one or more substituents selected from Cl, F, ═O, methyl, ethyl and methoxy. In this embodiment, R⁵ is methyl; R⁸ is methyl; m′ is 2 or 3 and ‘p’ is 2.

According to yet another embodiment, specifically provided are compounds of formula (I), in which R³ is hydrogen, methyl, ethyl, propyl, isopropyl, isobutyl, isopentyl, trifluoroethyl, —CH₂CH₂OCH₃, —CH₂CH₂CH₂OCH₃, —CH₂CH₂OH, —CH₂CH₂CH₂OH, —CH₂CH₂N(CH₃)₂,

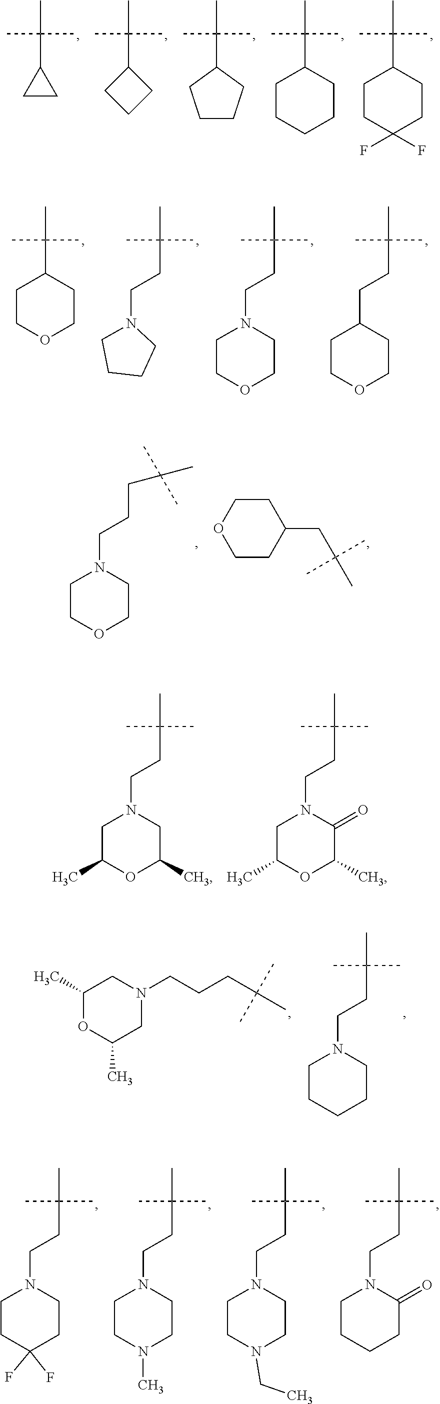

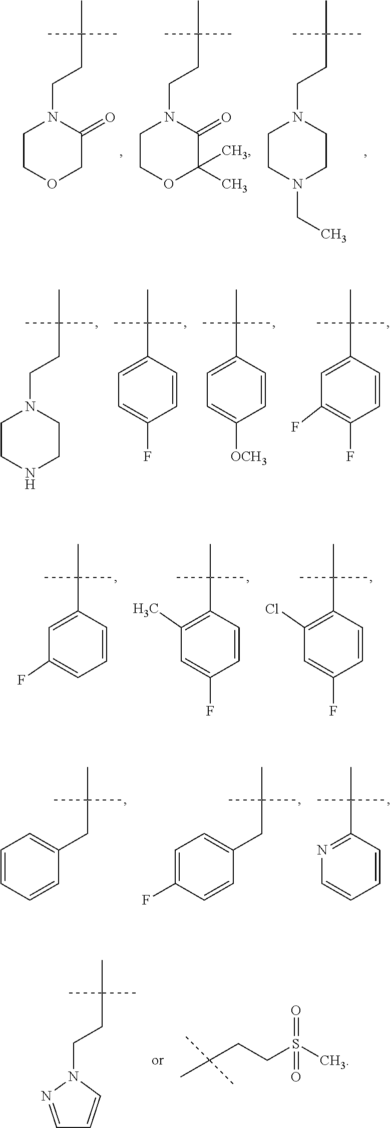

According to yet another embodiment, specifically provided are compounds of formula (I), in which R⁴ is hydrogen.

According to yet another embodiment, specifically provided are compounds of formula (I), in which ring A is

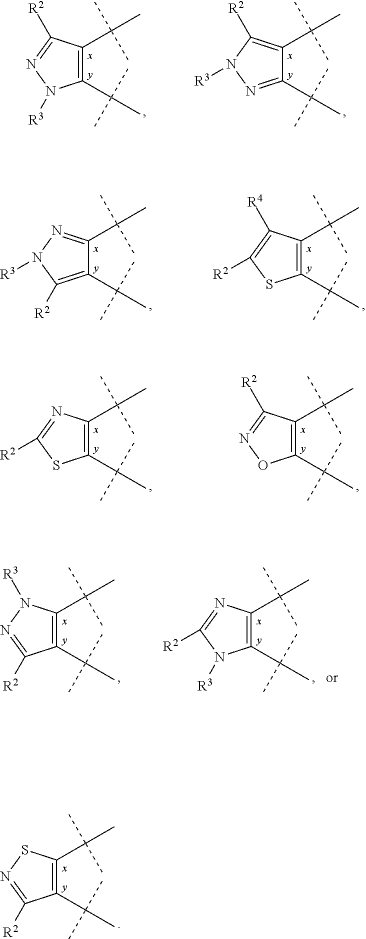

In this embodiment, R² is hydrogen, C₁₋₈alkyl (e.g. methyl, ethyl, isopropyl or isobutyl), haloC₁₋₈alkyl (e.g. trifluoromethyl or difluoromethyl), hydroxyC₁₋₈alkyl (e.g. —CH₂OH), —(CH₂)ₘOR⁵ (e.g. —CH₂OCH₃), —(CH₂)ₘC(O)NR⁵R⁶ (e.g. —C(O)NH₂), 3- to 15-membered heterocyclyl

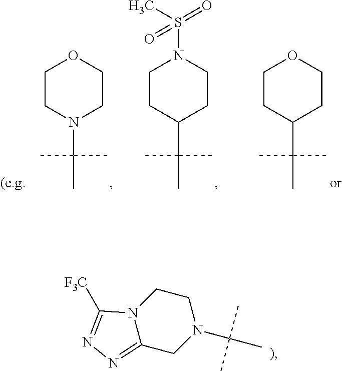

3- to 15-membered heterocyclylC₁₋₈alkyl

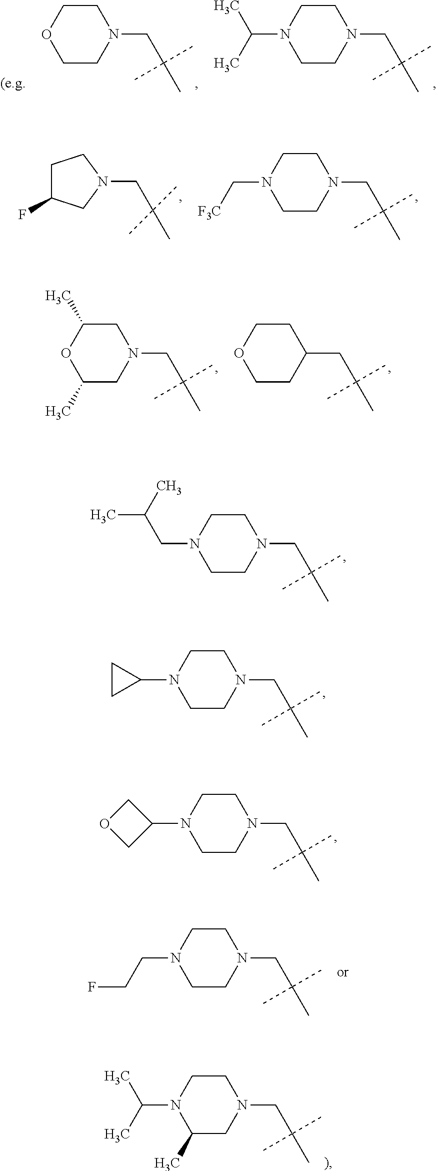

### C₆₋₁₄aryl

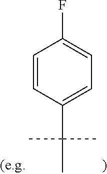

or C₆₋₁₄arylC₁₋₈alkyl

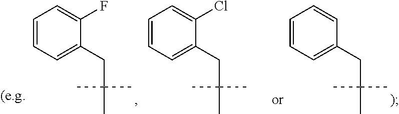

R³ is hydrogen, C₁₋₈alkyl (e.g. methyl, ethyl, propyl, isopropyl, isobutyl or isopentyl), haloC₁₋₈alkyl (e.g. trifluoroethyl), —(CH₂)ₘOR⁵ (e.g. —CH₂CH₂OCH₃ or —CH₂CH₂CH₂OCH₃), hydroxyC₁₋₈alkyl (e.g. —CH₂CH₂OH or —CH₂CH₂CH₂OH), —(CH)₂N(R⁵)₂ (e.g. —CH₂CH₂N(CH₃)₂), C₃₋₁₂cycloalkyl

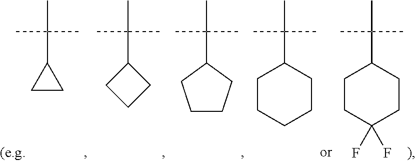

3- to 15-membered heterocyclyl

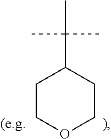

3- to 15-membered heterocyclylC₁₋₈alkyl

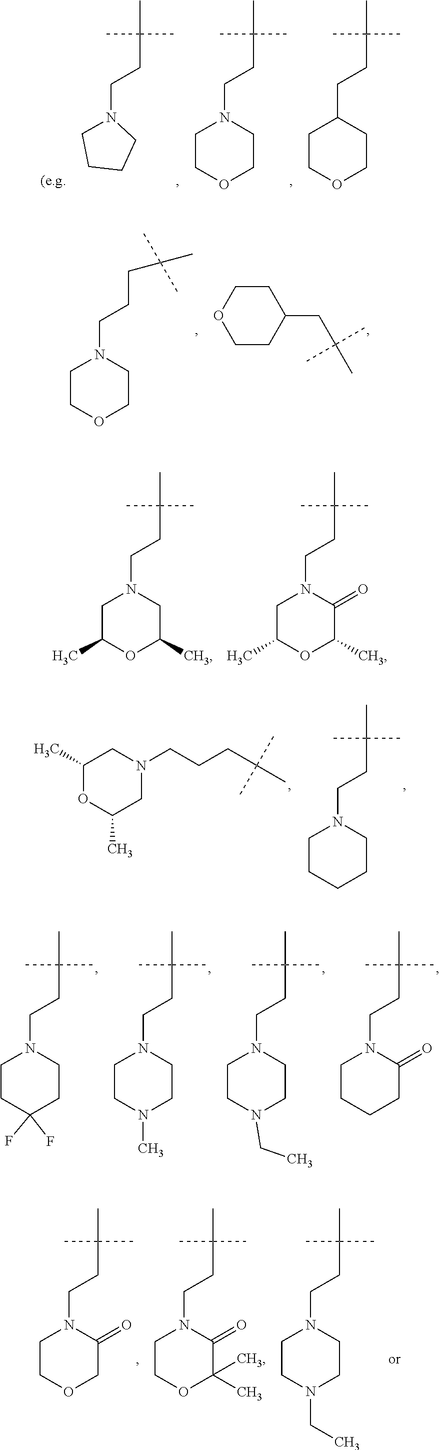

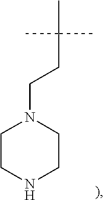

### C₆₋₁₄aryl

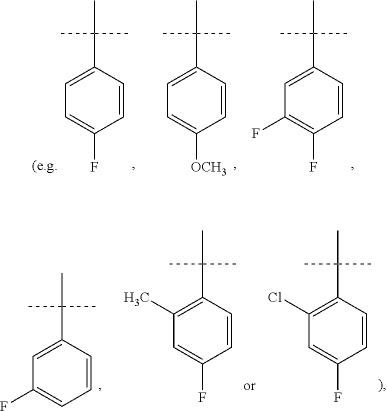

C₆₋₁₄arylC₁₋₈alkyl

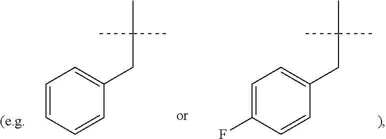

5- to 14-membered heteroaryl

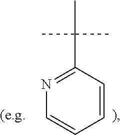

5- to 14-membered heteroarylC₁₋₈alkyl

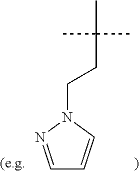

or —(CH₂)ₘS(O)_{P}R⁸

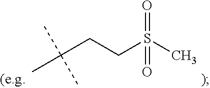

R⁴ is hydrogen; ‘m’ is 0, 1, 2 or 3 and ‘p’ is 2.

According to yet another embodiment, specifically provided are compounds of formula (I), in which ring A is

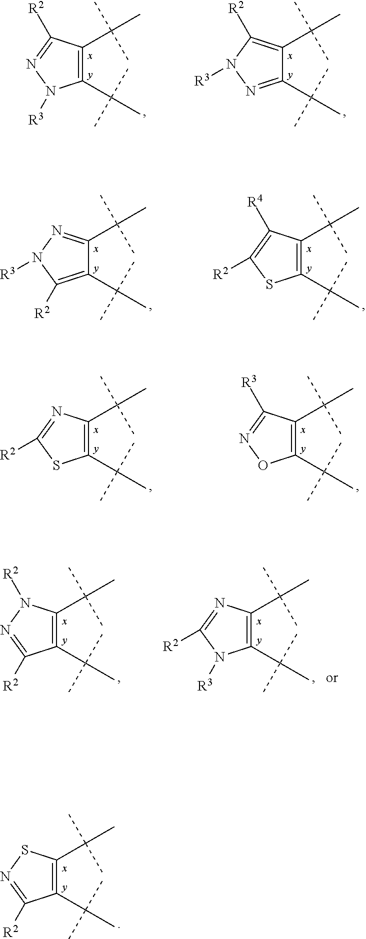

In this embodiment, R² is hydrogen, methyl, ethyl, isopropyl, isobutyl, trifluoromethyl, difluoromethyl, —CH₂OH, —CH₂OCH₃, —C(O)NH₂,

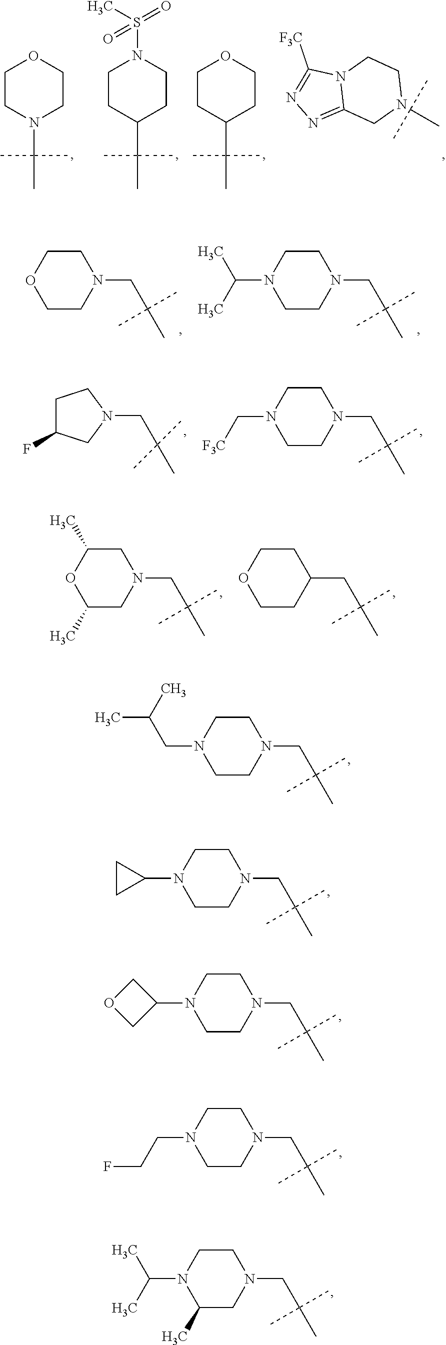

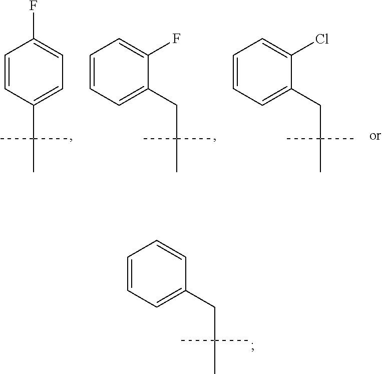

R³ is hydrogen, methyl, ethyl, propyl, isopropyl, isobutyl, isopentyl, trifluoroethyl, —CH₂CH₂OCH₃, —CH₂CH₂CH₂OCH₃, —CH₂CH₂OH, —CH₂CH₂CH₂OH, —CH₂CH₂N(CH₃)₂,

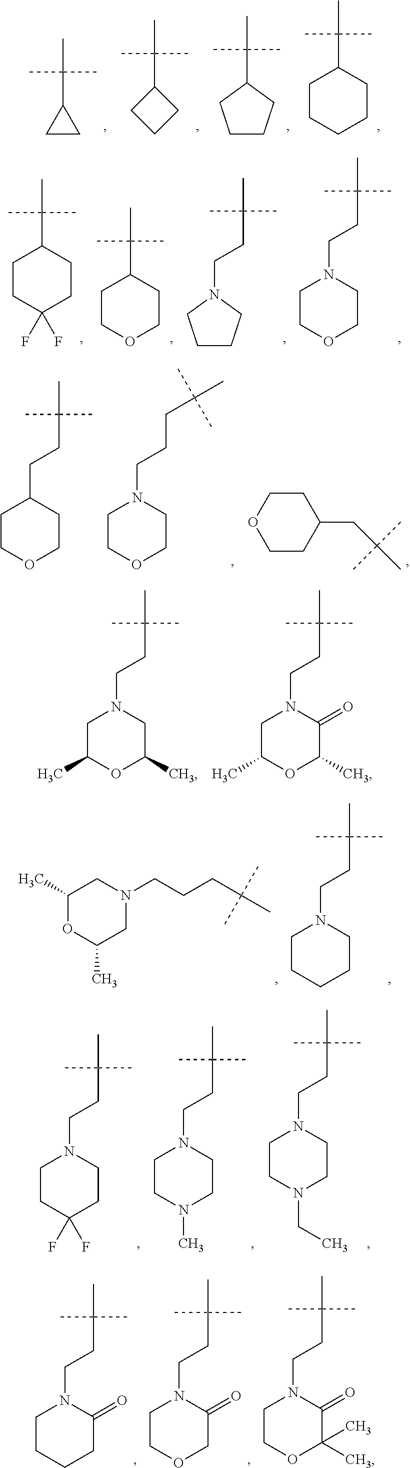

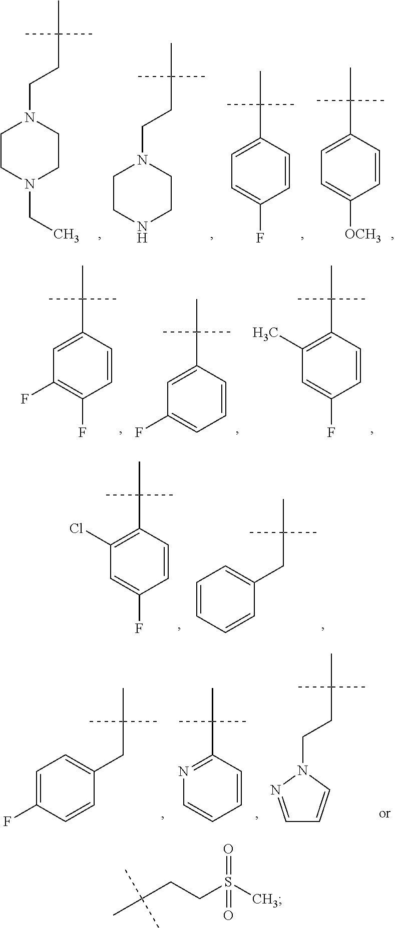

R⁴ is hydrogen; ‘m’ is 0, 1, 2 or 3 and ‘p’ is 2.

According to yet another embodiment, specifically provided are compounds of formula (I), in which ring A is

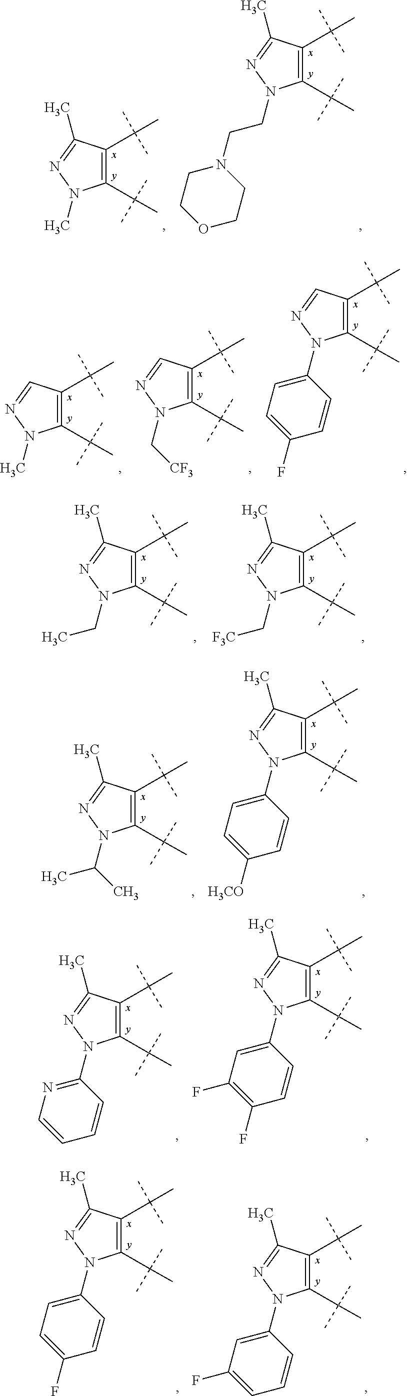

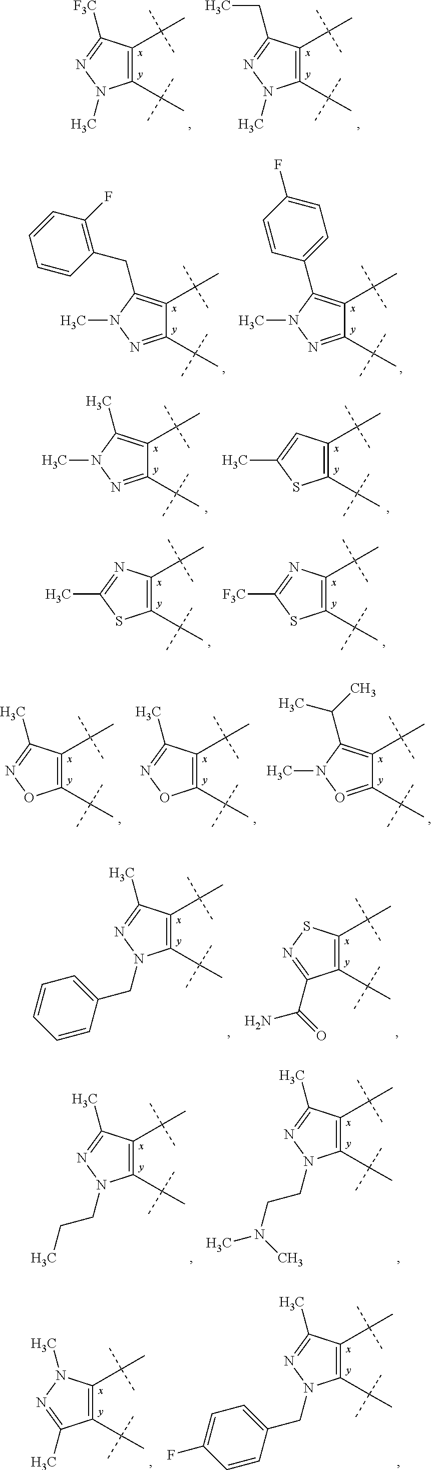

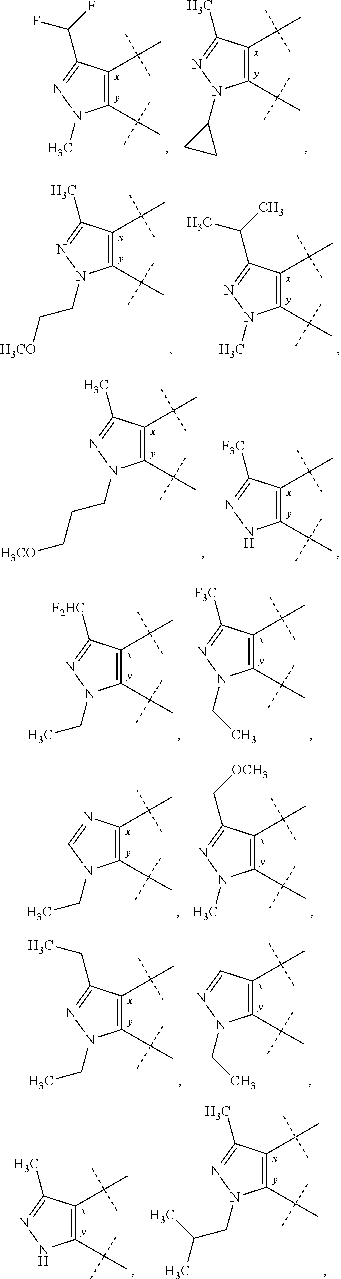

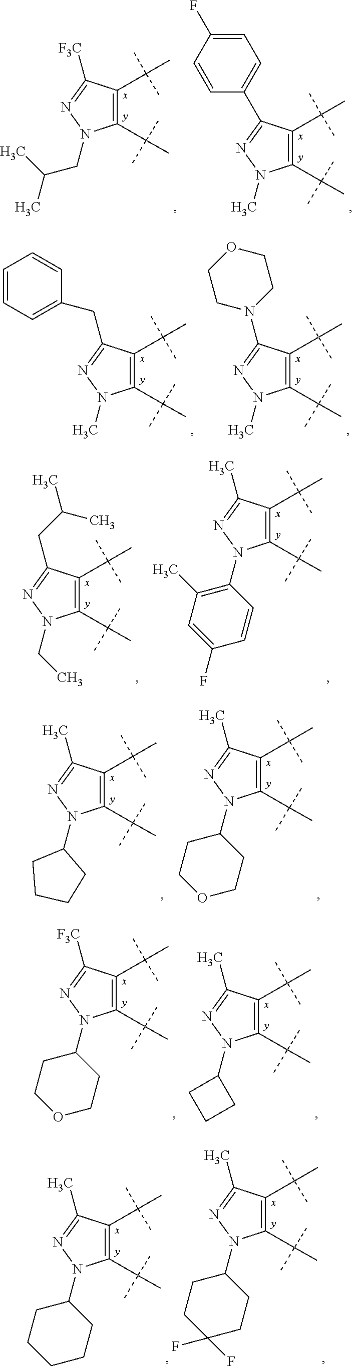

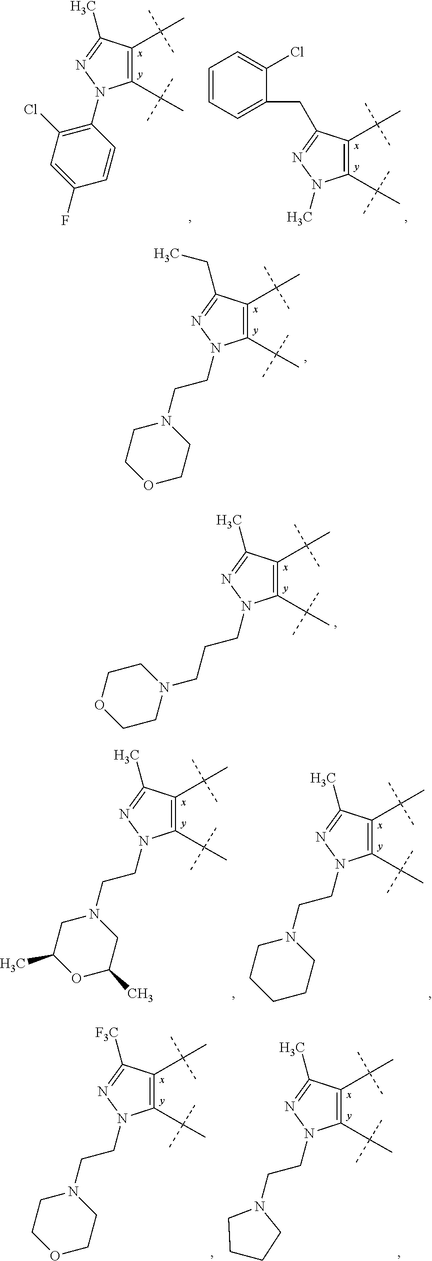

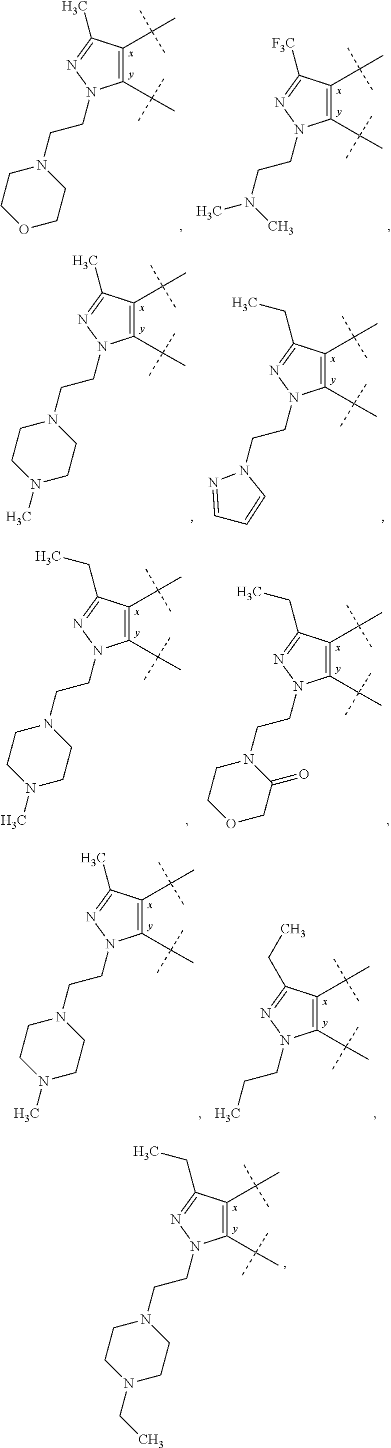

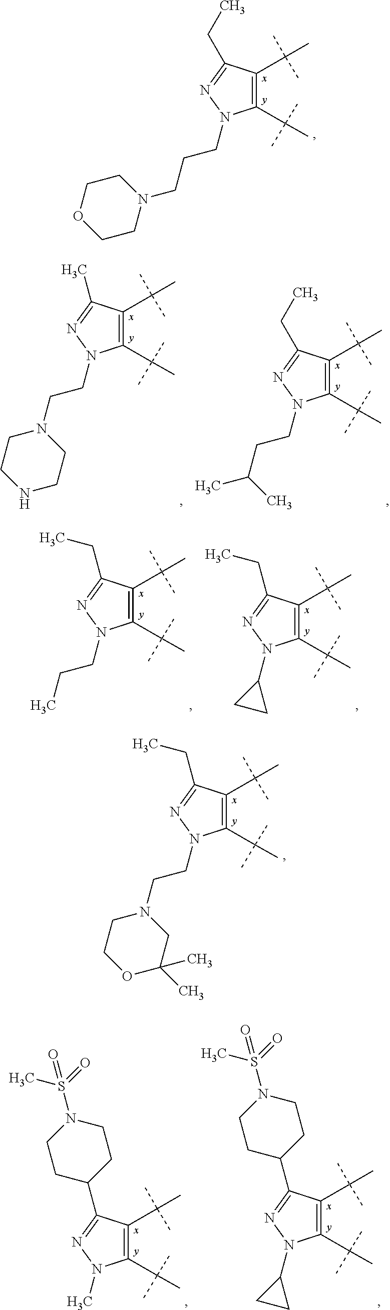

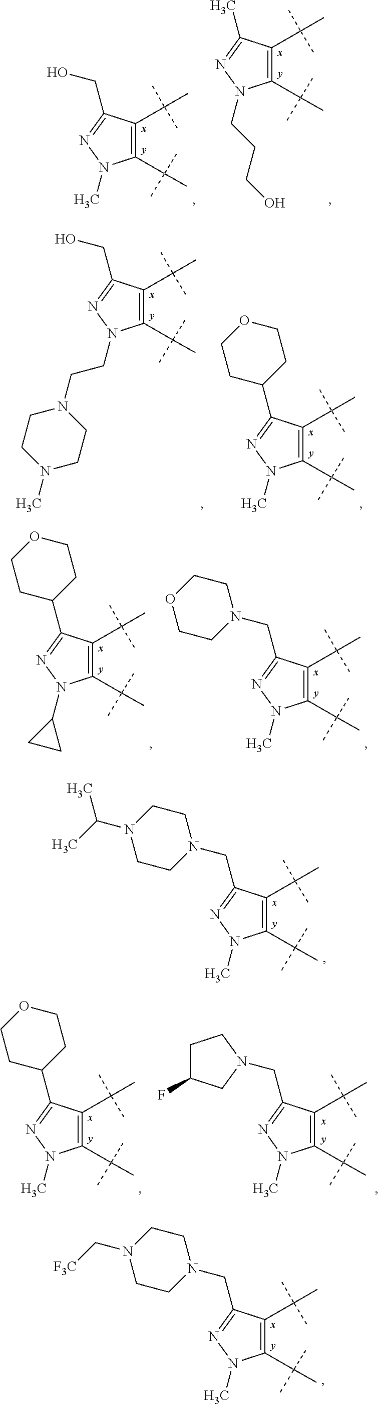

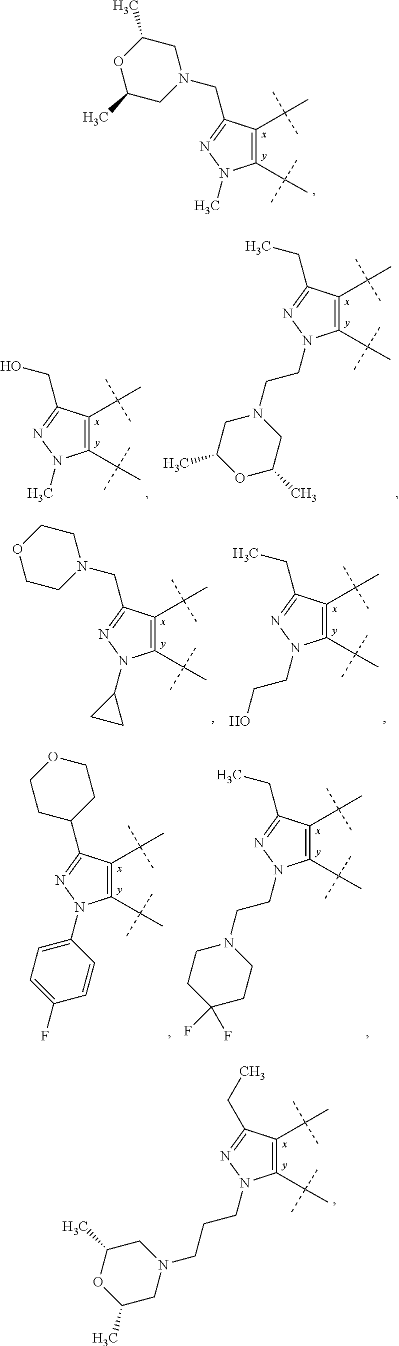

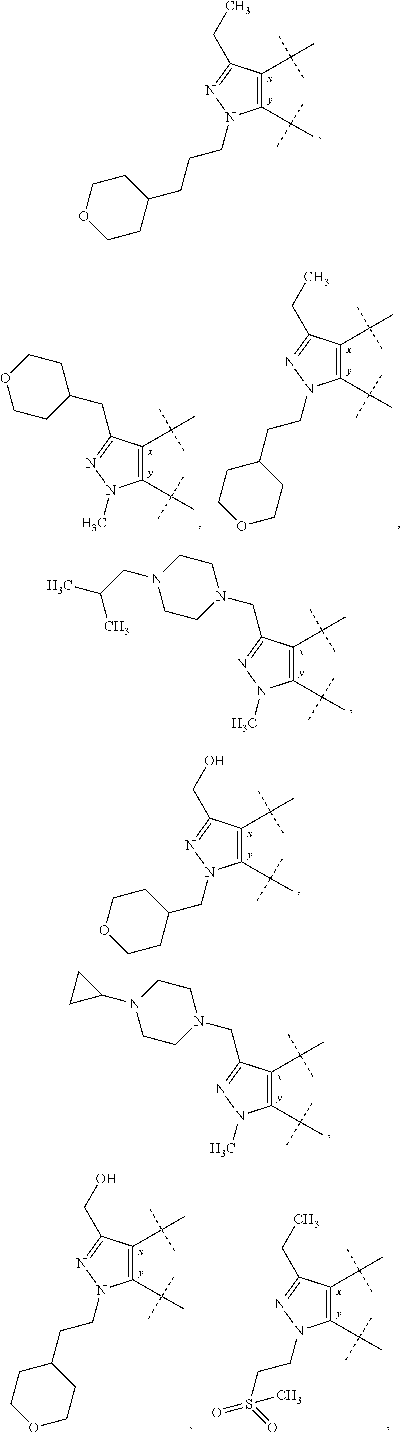

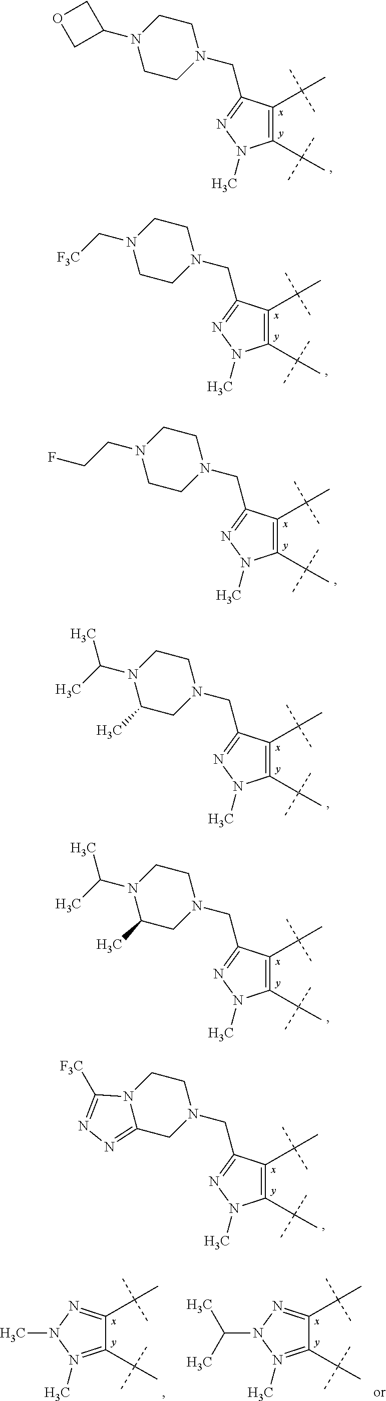

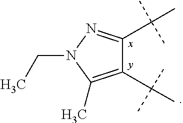

According to yet another embodiment, specifically provided are compounds of formula (I), in which

R is hydrogen, methyl or —C(O)CH₃;

X is NH or O;

Z¹ is CH or S, Z² is CH, Z³ is CH or N, Z⁴ is CH and Z⁵ is CH or absent;

R¹ is F, Cl, NH₂, OH, methyl, methoxy, —OCH₂CH₂OCH₃, CF₃, OCF₃,

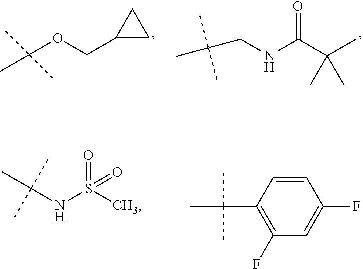

### or 1H-imidazol-1-yl;

ring A is

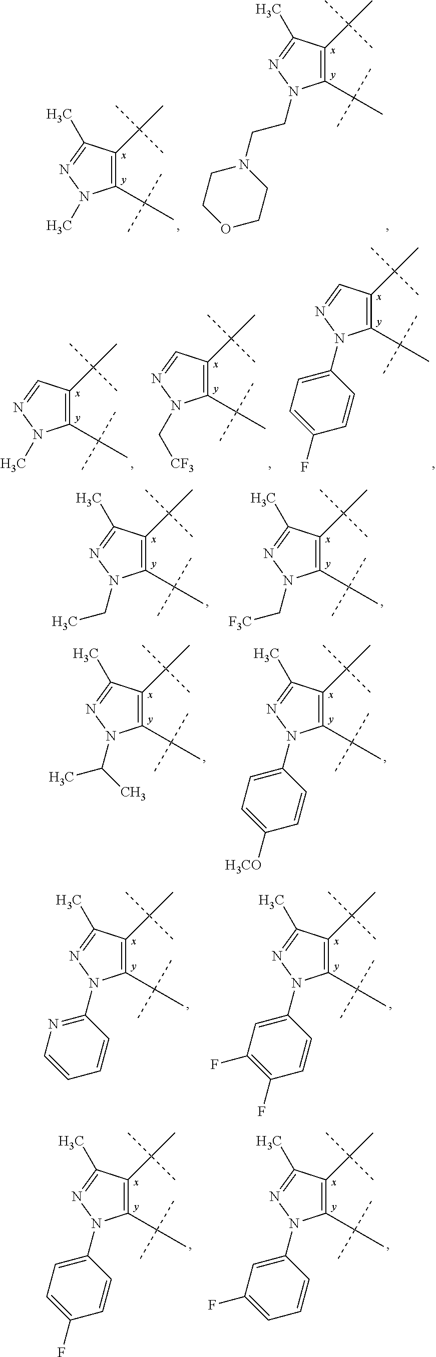

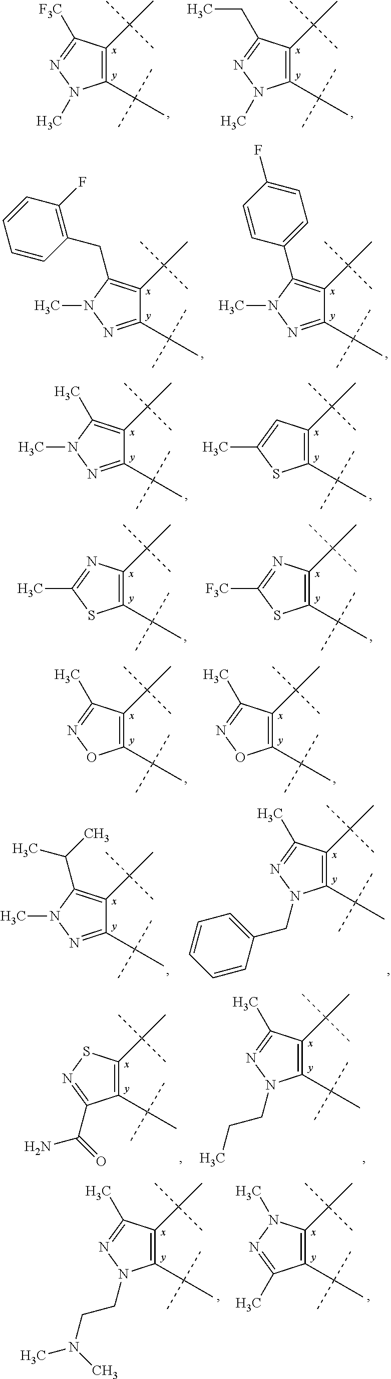

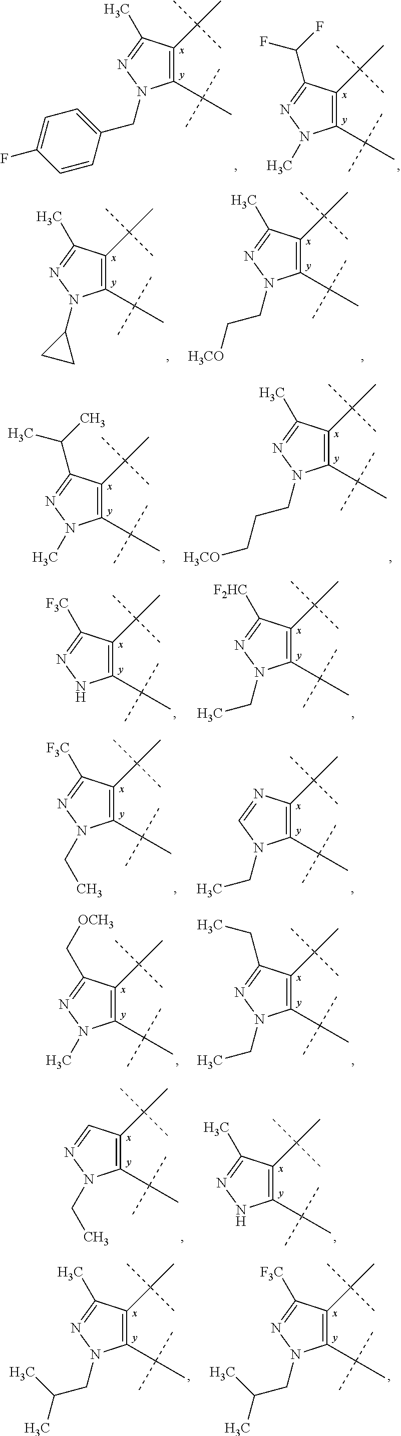

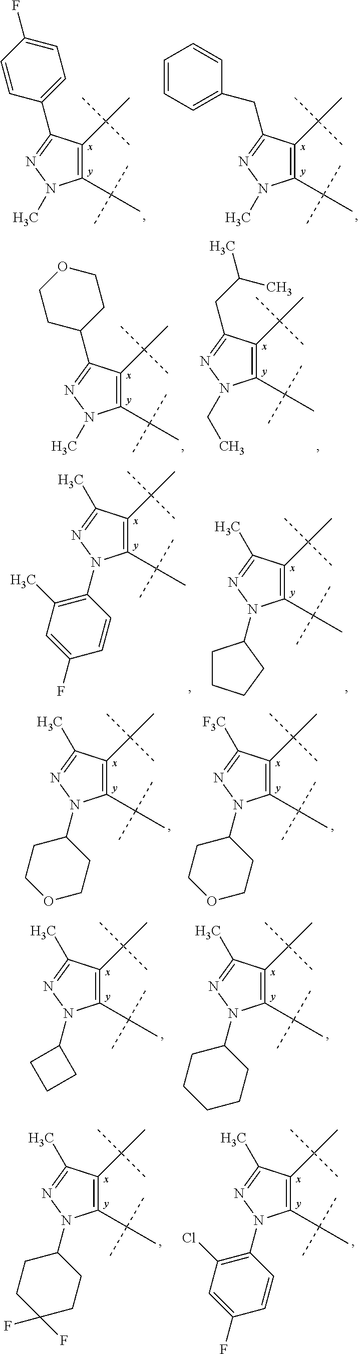

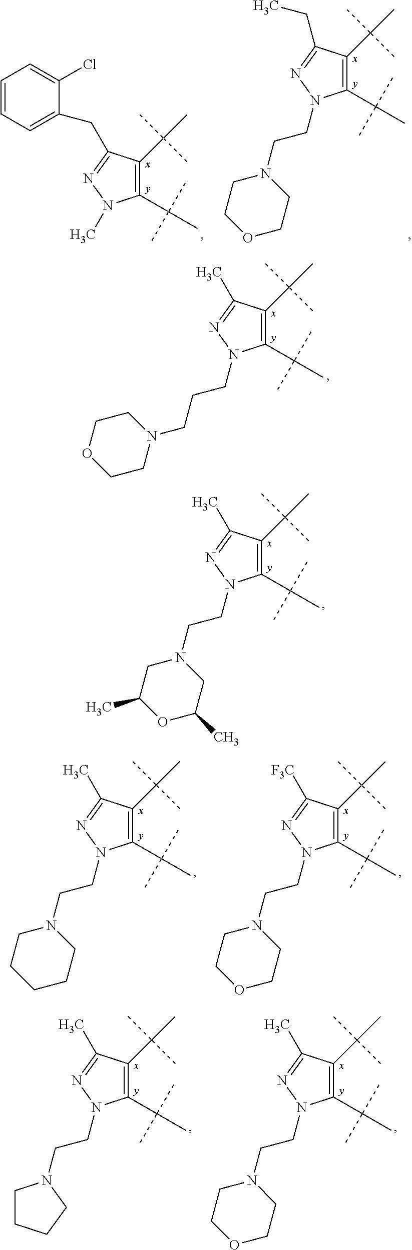

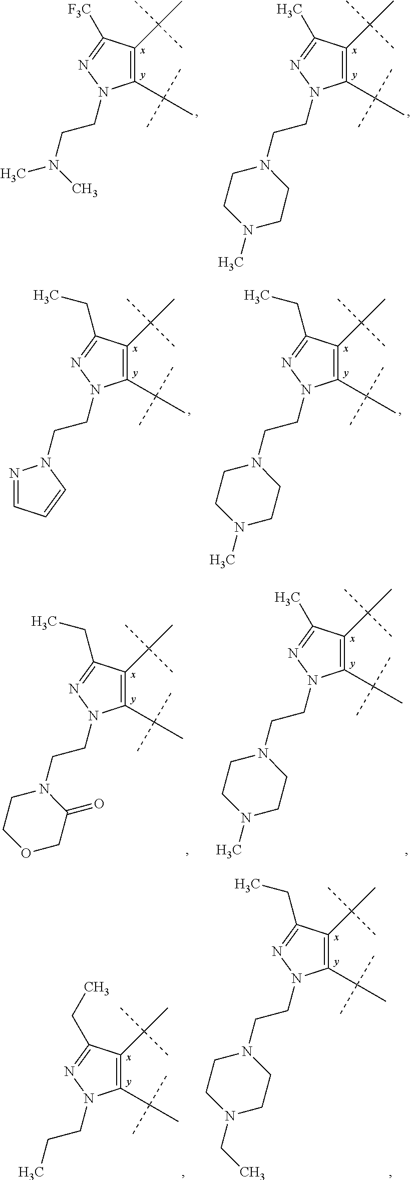

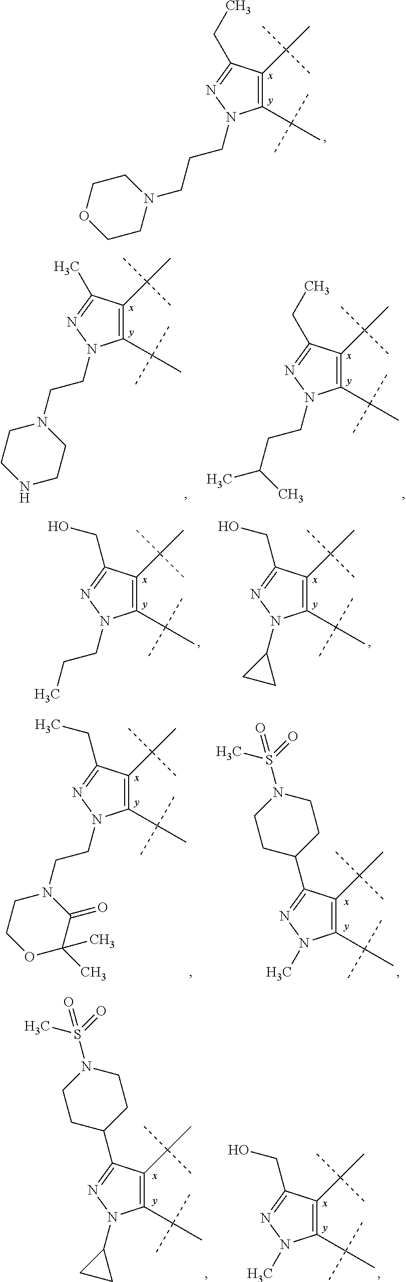

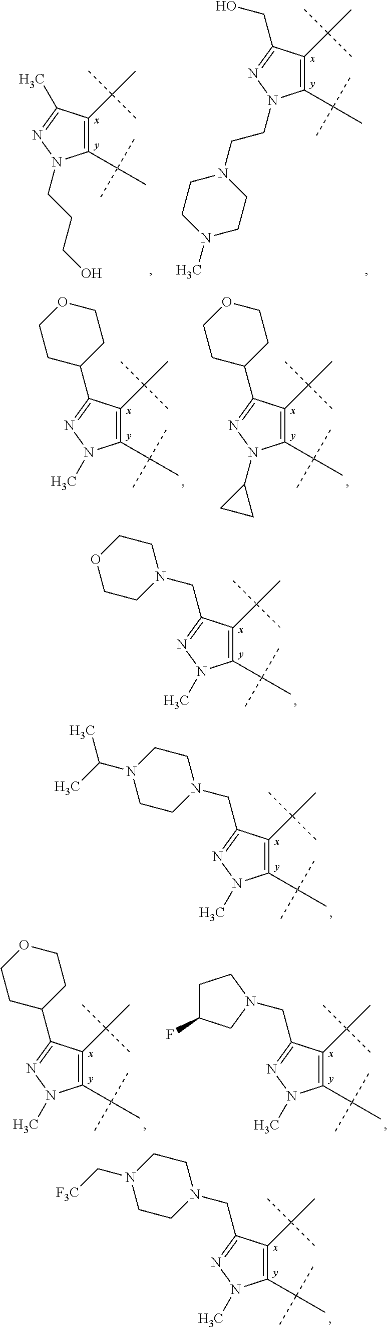

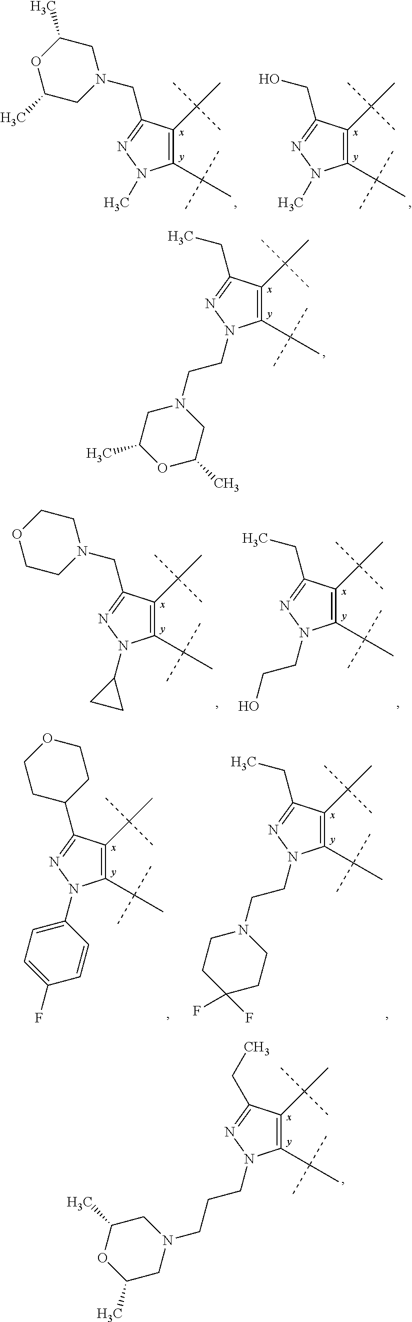

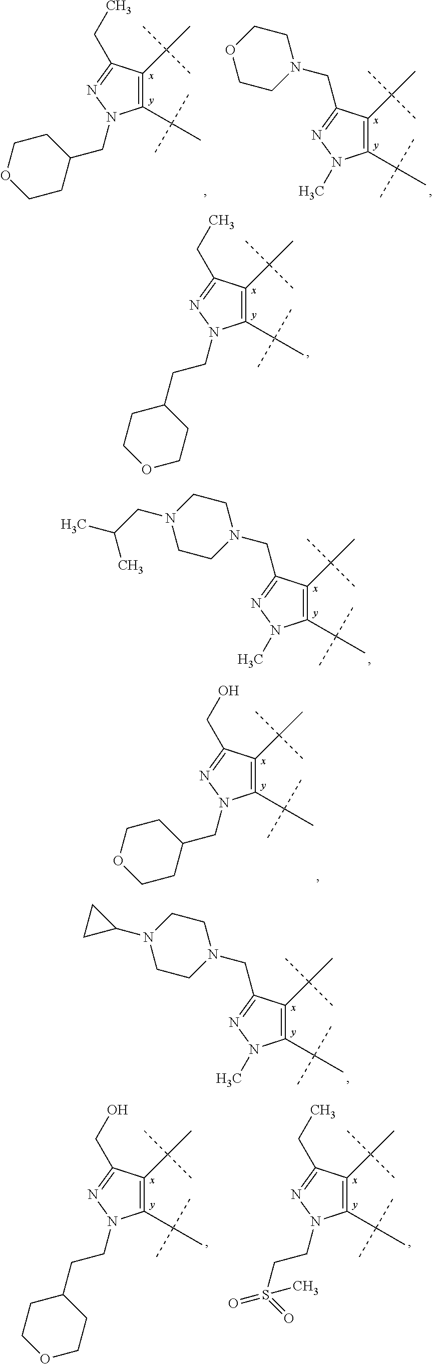

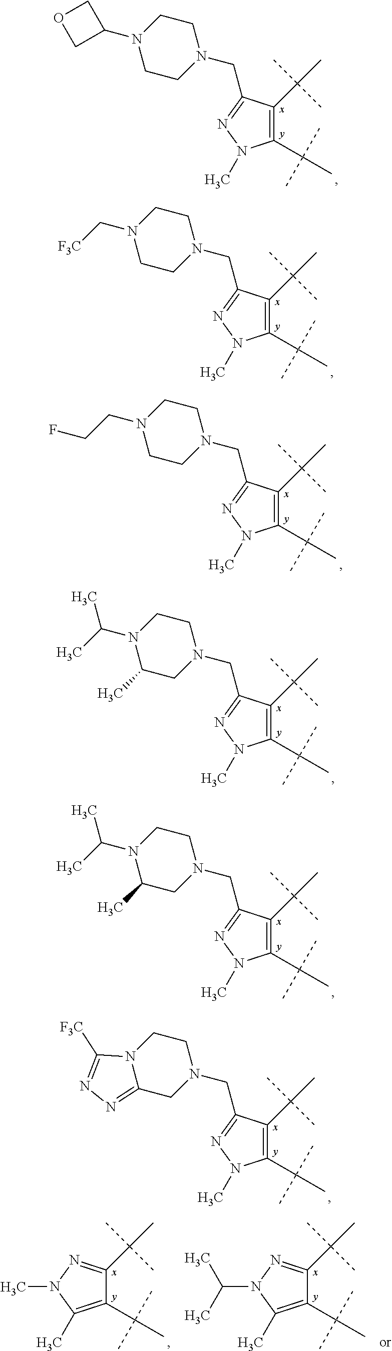

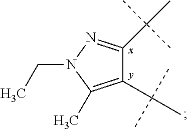

and

‘n’ is 0, 1, 2 or 3;

According to yet another embodiment, specifically provided are compounds of formula (I), in which

R is hydrogen;

X is NH;

Z¹ is CH, Z² is CH, Z³ is CH or N, Z⁴ is CH and Z⁵ is CH;

R¹ is F, Cl, NH₂, OH, methyl, methoxy, —OCH₂CH₂OCH₃, CF3, OCF3,

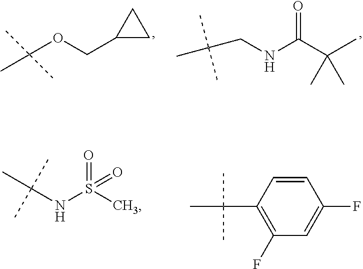

### or 1H-imidazol-1-yl;

ring A is

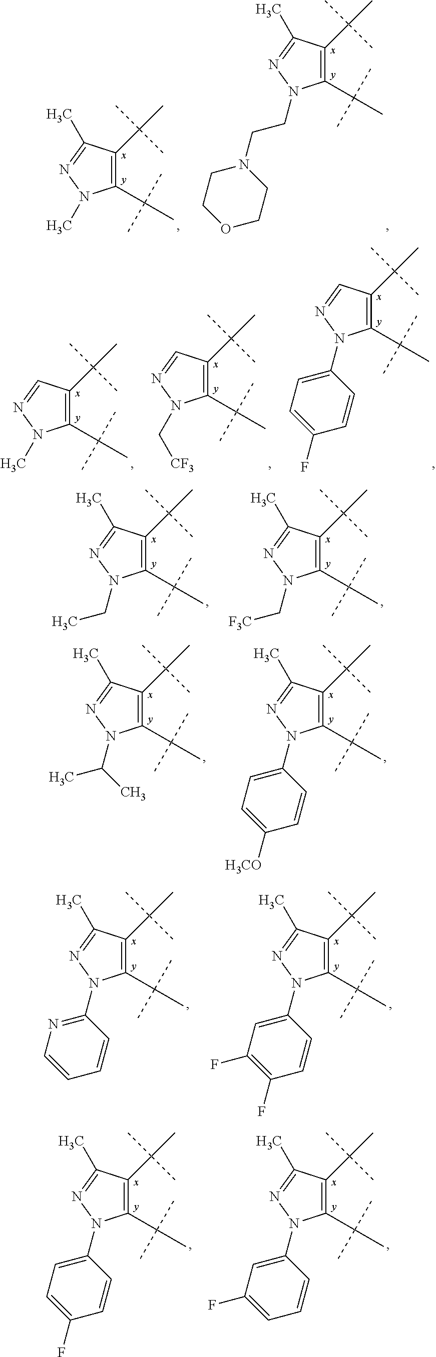

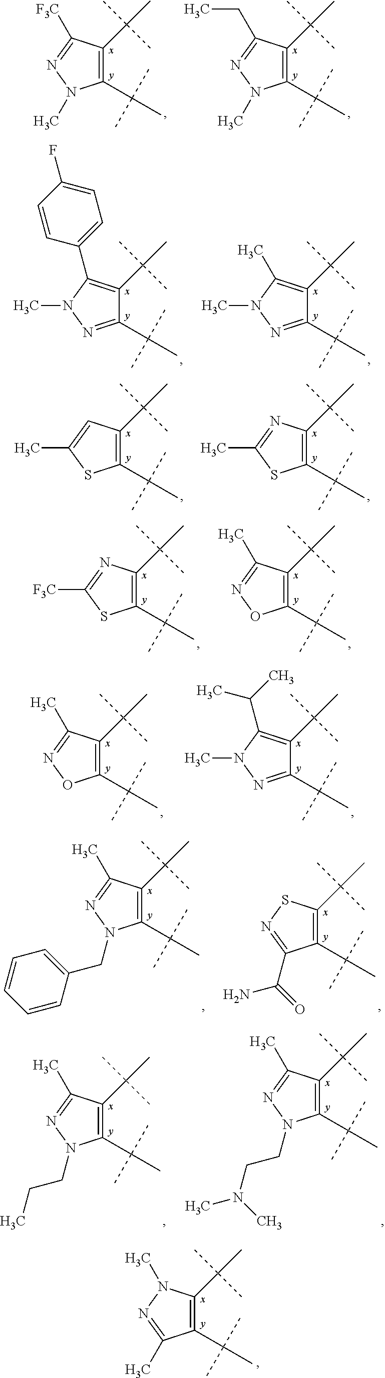

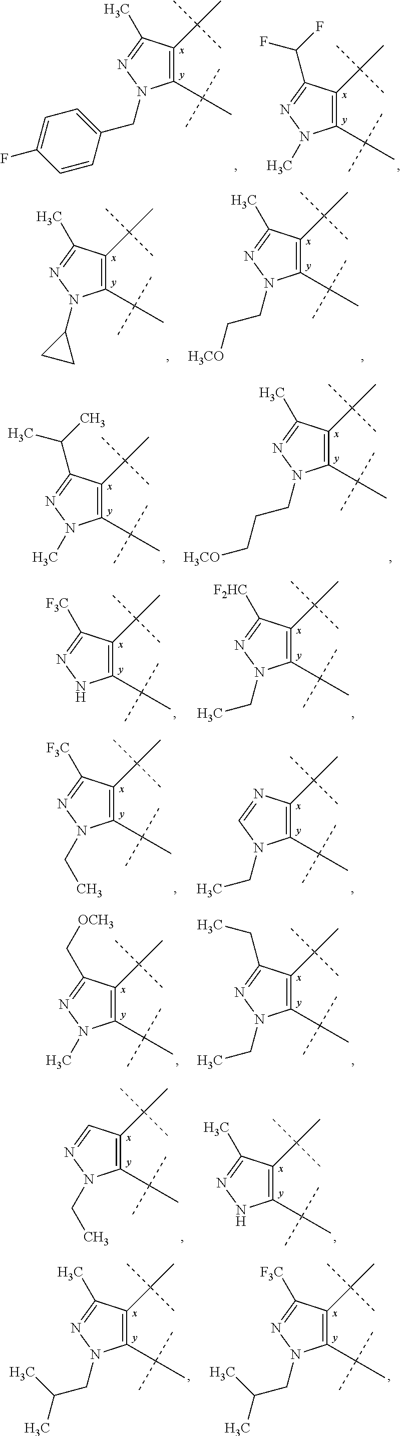

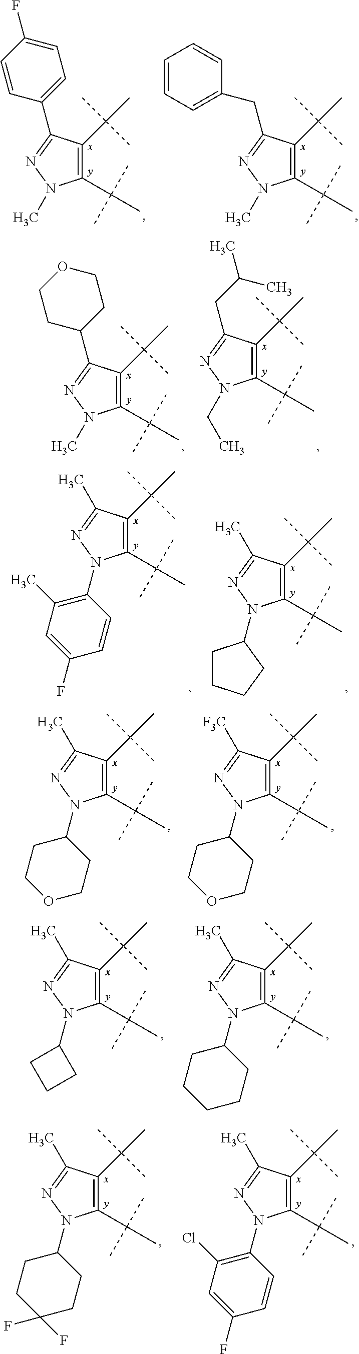

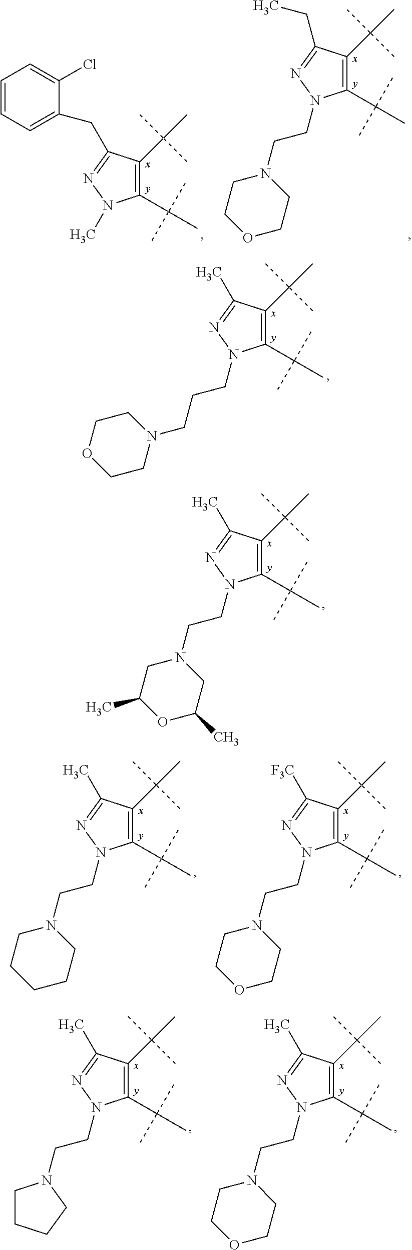

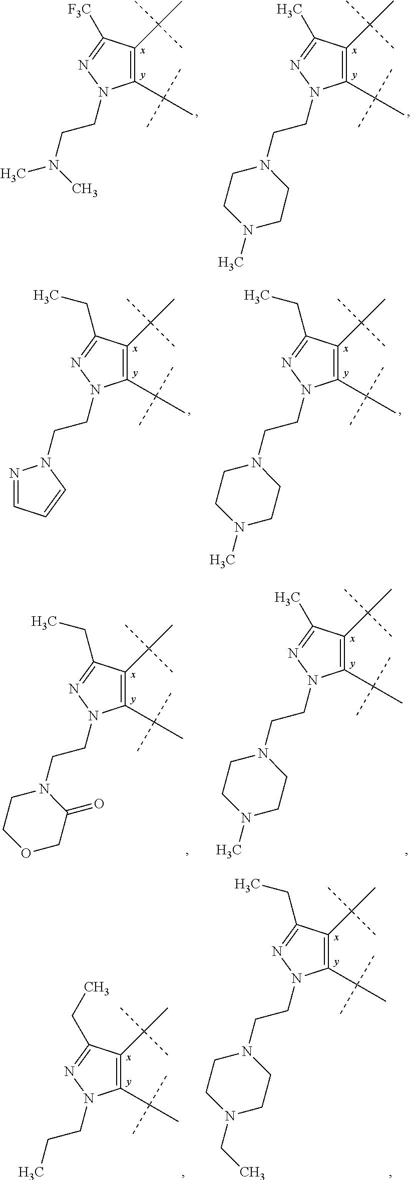

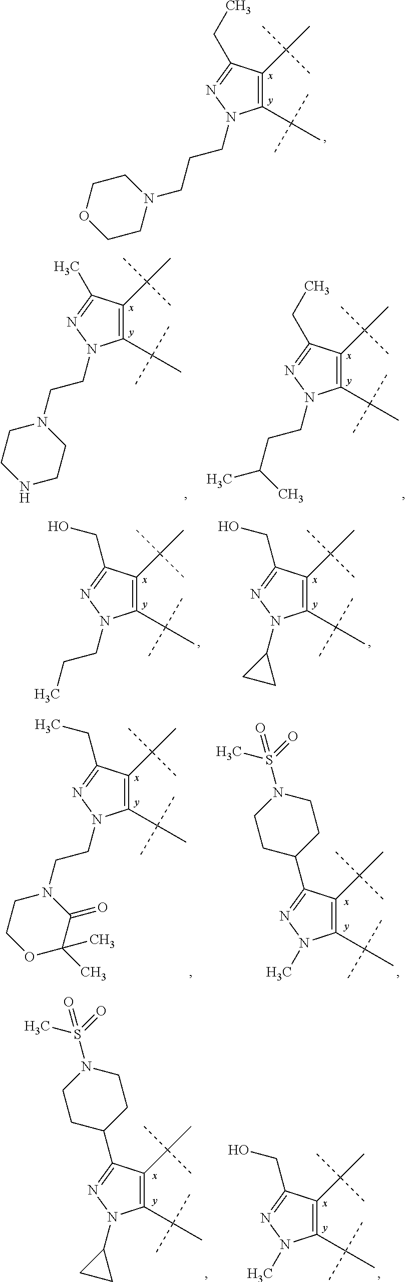

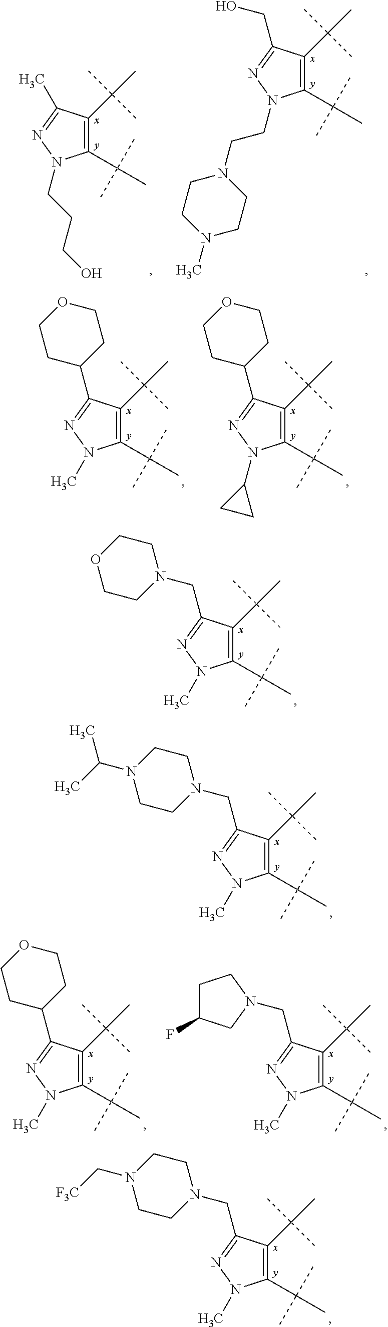

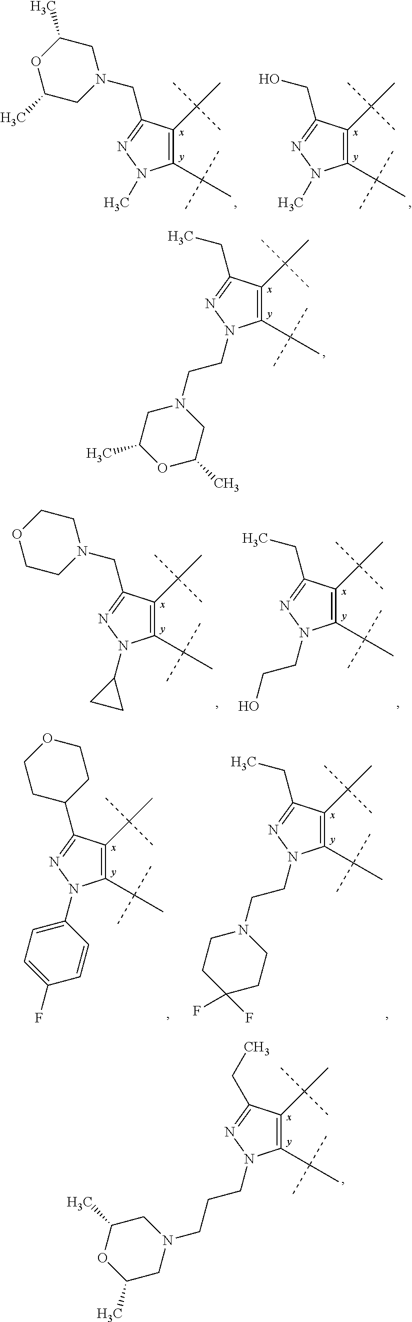

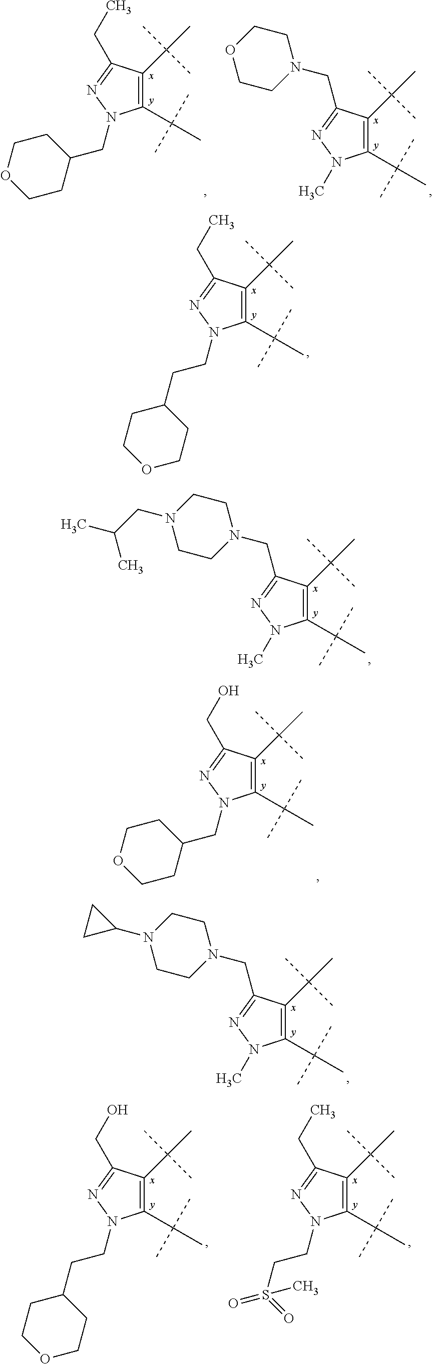

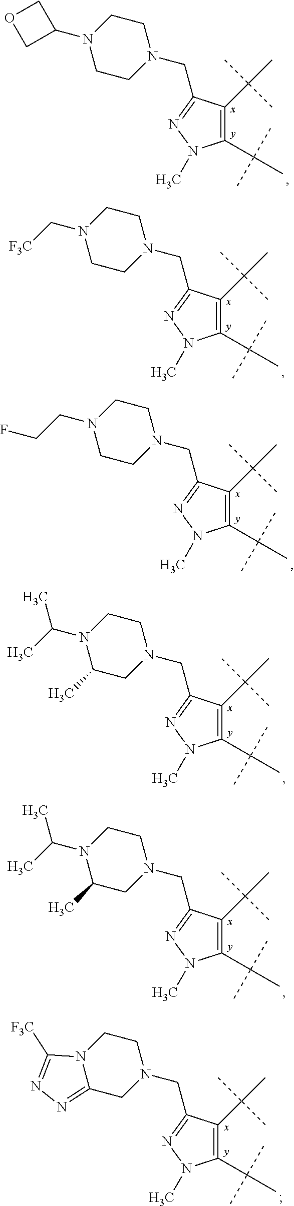

and

‘n’ is 0, 1, 2 or 3;

According to an embodiment, specifically provided are compounds of formula (I) with an IC₅₀ value of less than 1100 nM, preferably, less than 100 nM, more preferably less than 50 nM, with respect to NADPH oxidase inhibitor activity.

Further embodiments relating to groups X, R¹, R², R³, Z¹, Z², Z³, Z⁴, Z⁵, ring A and n (and groups defined therein) are described hereinafter in relation to the compounds of formula (II). It is to be understood that these embodiments are not limited to use in conjunction with formula (II), but apply independently and individually to the compounds of formula (I). For example, in an embodiment described hereinafter, the invention specifically provides compounds of formula (II), in which X is NH and consequently, there is also provided a compound of formula (I) in which X is NH.

The invention also provides a compound of formula (II) which is an embodiment of a compound of formula (I).

Accordingly the invention provides a compound of formula (II)

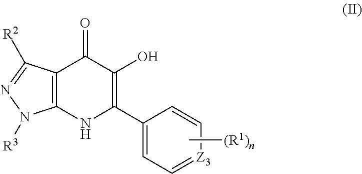

or a pharmaceutically acceptable salt thereof,
wherein,

Z₃ is CH or N;

at each occurrence, R¹ is independently selected from halogen, amino, hydroxyl, C₁₋₈alkyl, C₁₋₈alkoxy, C₁₋₈alkoxyC₁₋₈alkoxy, haloC₁₋₈alkyl, haloC₁₋₈alkoxy, —(CH₂)ₘNR⁵C(O)R⁶, —(CH₂)ₘOR⁵, —(CH₂)ₘNR⁷S(O)_{P}R⁸, C₆₋₁₄aryl and 5- to 14-membered heteroaryl; wherein C₆₋₁₄ aryl is optionally substituted with one or more substituents selected from halogen and C₁₋₈alkyl;

at each occurrence, R² is independently selected from hydrogen, C₁₋₈alkyl, haloC₁₋₈alkyl, hydroxyC₁₋₈alkyl, —(CH₂)ₘNR⁵C(O)NR⁶, —(CH₂)ₘOR⁵, 3- to 15-membered heterocyclyl, 3- to 15-membered heterocyclylC₁₋₈alkyl, C₆₋₁₄aryl and C₆₋₁₄arylC₁₋₈alkyl; wherein 3- to 15-membered heterocyclyl, 3- to 15-membered heterocyclylC₁₋₈alkyl, C₆₋₁₄aryl and C₆₋₁₄arylC₁₋₈alkyl are optionally substituted with one or more substituents selected from halogen, C₁₋₈alkyl, haloC₁₋₈alkyl, —(CH₂)ₘS(O)_{P}R⁸,C₃₋₁₂cycloalkyl and 3- to 15-membered heterocyclyl;

at each occurrence, R³ is independently selected from hydrogen, C₁₋₈alkyl, haloC₁₋₈alkyl, hydroxyC₁₋₈alkyl, —(CH₂)ₘOR⁵, —(CH)₂N(R⁵)₂, —(CH₂)ₘS(O)_{P}R⁸, C₃₋₁₂cycloalkyl, 3- to 15-membered heterocyclyl, 3- to 15-membered heterocyclylC₁₋₈alkyl, C₆₋₁₄aryl, C₆₋₁₄arylC₁₋₈alkyl, 5- to 14-membered heteroaryl and 5- to 14-membered heteroarylC₁₋₈alkyl; wherein C₃₋₁₂cycloalkyl, 3- to 15-membered heterocyclylC₁₋₈alkyl, C₆₋₁₄aryl and C₆₋₁₄arylC₁₋₈alkyl are optionally substituted with one or more substituents selected from halogen, oxo, C₁₋₈alkyl and C₁₋₈alkoxy;

at each occurrence, R⁵ is independently selected from hydrogen and C₁₋₈alkyl;

at each occurrence, R⁶ is independently selected from hydrogen and C₁₋₈alkyl;

at each occurrence, R⁷ is independently selected from hydrogen and C₁₋₈alkyl;

at each occurrence, R⁸ is independently selected from hydrogen and C₁₋₈alkyl;

‘m’ is an integer ranging from 0 to 4, both inclusive;

‘n’ is an integer ranging from 0 to 5, both inclusive; and

‘p’ is an integer ranging from 0 to 2, both inclusive.

The compounds of formula (II) may involve one or more embodiments. It is to be understood that the embodiments below are illustrative of the present invention and are not intended to limit the claims to the specific embodiments exemplified. It is also to be understood that the embodiments defined herein may be used independently or in conjunction with any definition of any other embodiment defined herein. Thus the invention contemplates all possible combinations and permutations of the various independently described embodiments. For example, the invention provides compounds of formula (II) as defined above wherein Z₃ is CH (according to an embodiment defined below), Z₃ is N (according to another embodiment defined below). ‘n’ is 0, 1, 2 or 3 (according to yet another embodiment defined below).

According to one embodiment, specifically provided are compounds of formula (II), in which Z₃ is CH.

According to another embodiment, specifically provided are compounds of formula (II), in which Z₃ is N.

According to yet another embodiment, specifically provided are compounds of formula (II), in which R¹ is halogen (e.g. F, Cl, or Br), amino (e.g. NH₂), hydroxyl (e.g. OH), C₁₋₈alkyl (e.g. methyl), C₁₋₈alkoxy (e.g methoxy), C₁₋₈alkoxyC₁₋₈alkoxy (e.g. —OCH₂CH₂OCH₃), haloC₁₋₈alkyl (e.g. CF₃), haloC₁₋₈alkoxy (e.g OCF₃), —(CH₂)ₘOR⁵

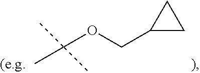

—(CH₂)ₘNR⁵C(O)R⁶

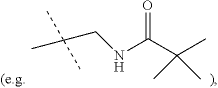

—(CH₂)ₘNR⁷S(O)_{P}R⁸

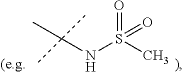

C₆₋₁₄aryl (e.g. phenyl) optionally substituted with one or more substituents selected from halogen (e.g. Cl, F or Br) and 5- to 14-membered heteroaryl (e.g. 1H-imidazol-1-yl). In this embodiment, R⁵ is hydrogen or C₃₋₁₂cycloalkylC₁₋₈alkyl

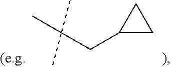

R⁶ is C₁₋₈alkyl

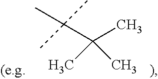

R⁷ is hydrogen, R⁸ is C₁₋₈alkyl (e.g. methyl), ‘p’ is 2 and ‘m’ is 0 or 1.

According to yet another embodiment, specifically provided are compounds of formula (II), in which R¹ is halogen (e.g. F, Cl, or Br), amino (e.g. NH₂), hydroxyl (e.g. OH), C₁₋₈alkyl (e.g. methyl), C₁₋₈alkoxy (e.g methoxy), C₁₋₈alkoxyC₁₋₈alkoxy (e.g. —OCH₂CH₂OCH₃), haloC₁₋₈alkyl (e.g. CF3), haloC₁₋₈alkoxy (e.g OCF3), —(CH₂)ₘOR⁵

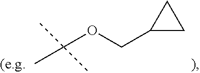

(CH₂)ₘNR⁵C(O)R⁶

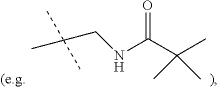

—(CH₂)ₘNR⁷S(O)_{P}R⁸

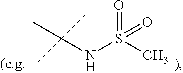

### C₆₋₁₄aryl

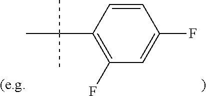

or 5- to 14-membered heteroaryl (e.g. 1H-imidazol-1-yl). In this embodiment, R⁵ is hydrogen or

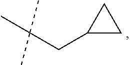

### R⁶ is

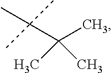

R⁷ is hydrogen, R⁸ is methyl, ‘p’ is 2 and ‘m’ is 0 or 1.

According to yet another embodiment, specifically provided are compounds of formula (II), in which R¹ is F, Cl, NH₂, OH, methyl, methoxy, —OCH₂CH₂OCH₃, CF3, OCF3,

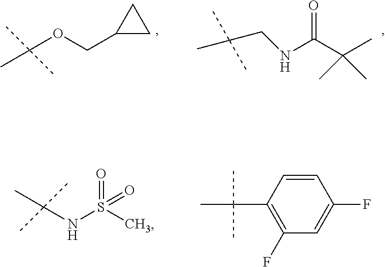

or 1H-imidazol-1-yl. According to yet another embodiment, specifically provided are compounds of formula (II), in which ‘n’ is 0, 1, 2 or 3.

According to yet another embodiment, specifically provided are compounds of formula (II), in which ‘n’ is 0.

According to yet another embodiment, specifically provided are compounds of formula (II), in which ‘n’ is 1.

According to yet another embodiment, specifically provided are compounds of formula (II), in which ‘n’ is 2.

According to yet another embodiment, specifically provided are compounds of formula (II), in which ‘n’ is 3.

According to yet another embodiment, specifically provided are compounds of formula (II), in which R¹ is F, Cl, NH₂, OH, methyl, methoxy, —OCH₂CH₂OCH₃, CF3, OCF3,

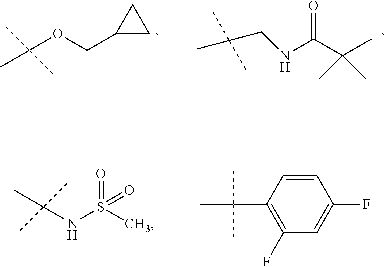

or 1H-imidazol-1-yl and ‘n’ is 0, 1, 2 or 3.

According to yet another embodiment, specifically provided are compounds of formula (II), in which R² is hydrogen, C₁₋₈alkyl (e.g. methyl, ethyl, isopropyl or isobutyl), haloC₁₋₈alkyl (e.g. trifluoromethyl or difluoromethyl), hydroxyC₁₋₈alkyl (e.g. —CH₂OH), —(CH₂)ₘOR⁵ (e.g. —CH₂OCH₃), —(CH₂)ₘC(O)NR⁵R⁶ (e.g. —C(O)NH₂), 3- to 15-membered heterocyclyl

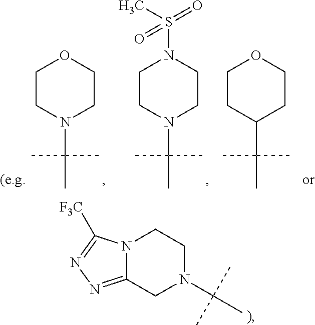

3- to 15-membered heterocyclylC₁₋₈alkyl

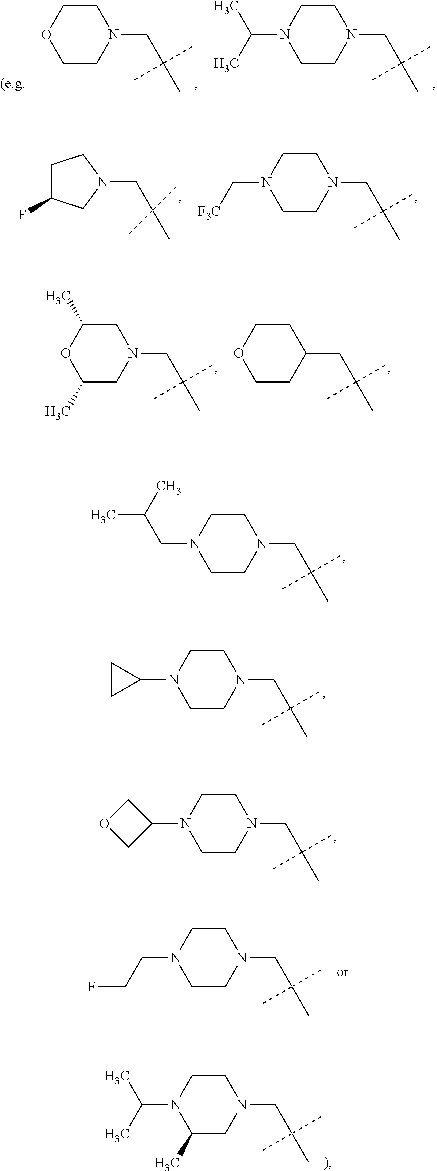

### C₆₋₁₄aryl

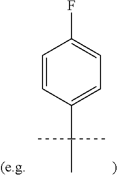

or C₆₋₁₄arylC₁₋₈alkyl

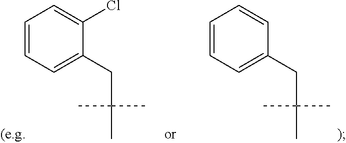

wherein 3- to 15-membered heterocyclyl, 3- to 15-membered heterocyclylC₁₋₈alkyl, C₆₋₁₄aryl and C₆₋₁₄arylC₁₋₈alkyl are optionally substituted with one or more substituents selected from halogen (e.g. Cl, F or Br), C₁₋₈alkyl (e.g. methyl, 2-methylpropyl or prop-2-yl), haloC₁₋₈alkyl (e.g. trifluoromethyl, trifluoroethyl or fluoroethyl), —(CH₂)ₘS(O)_{P}R⁸ (e.g. —S(O)₂CH₃), C₃₋₁₂cycloalkyl (e.g. cyclopropyl) and 3- to 15-membered heterocyclyl (e.g. oxatane). In this embodiment, R⁵ is hydrogen or C₁₋₈alkyl (e.g. methyl); R⁶ is hydrogen; ‘p’ is 2 and ‘m’ is 0 or 1.

According to yet another embodiment, specifically provided are compounds of formula (II), in which R² is hydrogen, C₁₋₈alkyl (e.g. methyl, ethyl, isopropyl or isobutyl), haloC₁₋₈alkyl (e.g. trifluoromethyl or difluoromethyl), hydroxyC₁₋₈alkyl (e.g. —CH₂OH), —(CH₂)ₘOR⁵ (e.g. —CH₂OCH₃), —(CH₂)ₘC(O)NR⁵R⁶ (e.g. —C(O)NH₂), 3- to 15-membered heterocyclyl

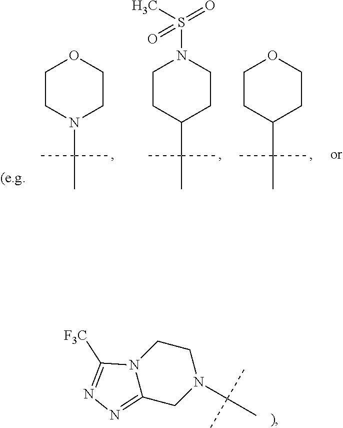

3- to 15-membered heterocyclylC₁₋₈alkyl

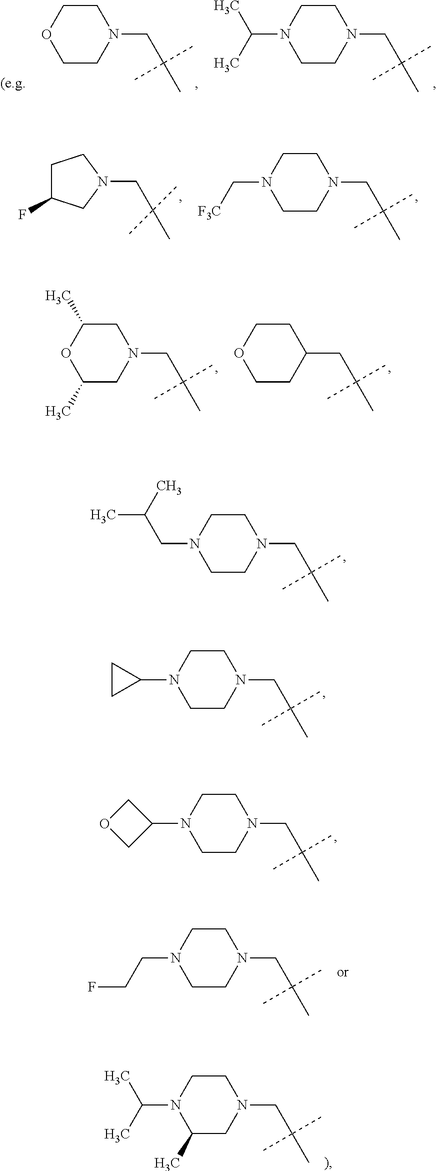

### C₆₋₁₄aryl

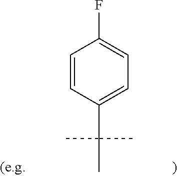

or C₆₋₁₄arylC₁₋₈alkyl

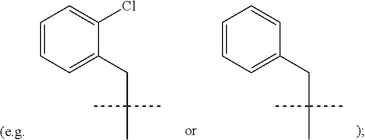

wherein 3- to 15-membered heterocyclyl, 3- to 15-membered heterocyclylC₁₋₈alkyl, C₆₋₁₄aryl and C₆₋₁₄arylC₁₋₈alkyl are optionally substituted with one or more substituents selected from Cl, methyl, 2-methylpropyl, trifluoromethyl, trifluoroethyl, fluoroethyl, —S(O)₂CH₃, cyclopropyl and oxatane. In this embodiment, R⁵ is hydrogen or methyl; R⁶ is hydrogen; ‘p’ is 2 and ‘m’ is 0 or 1.

According to yet another embodiment, specifically provided are compounds of formula (II), in which R² is hydrogen, methyl, ethyl, isopropyl, isobutyl, trifluoromethyl, difluoromethyl, —CH₂OH, —CH₂OCH₃, —C(O)NH₂),

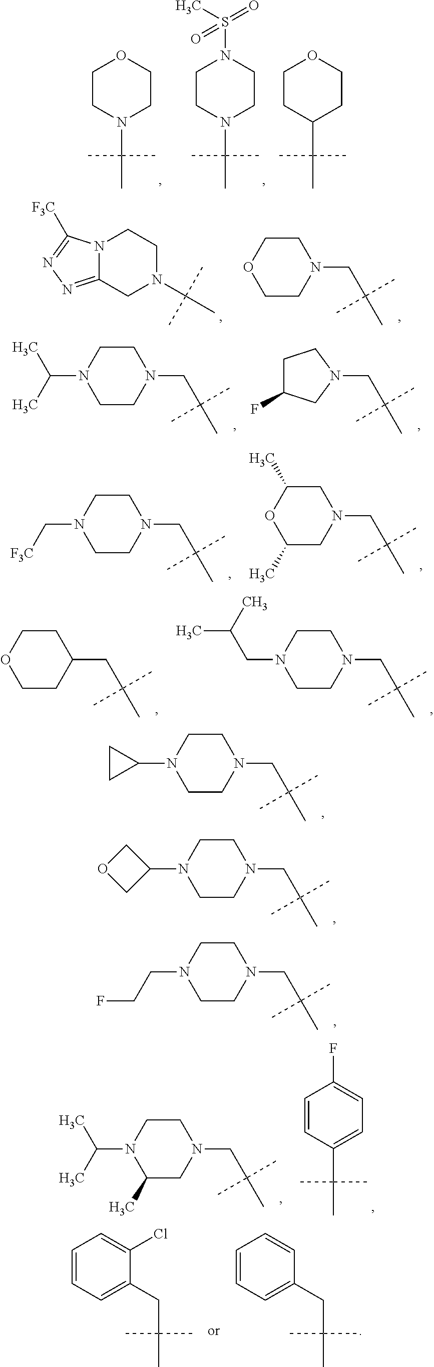

According to yet another embodiment, specifically provided are compounds of formula (II), in which R³ is hydrogen, C₁₋₈alkyl (e.g. methyl, ethyl, propyl, isopropyl, isobutyl or isopentyl), haloC₁₋₈alkyl (e.g. trifluoroethyl), —(CH₂)ₘOR⁵ (e.g. —CH₂CH₂OCH₃ or —CH₂CH₂CH₂OCH₃), hydroxyC₁₋₈alkyl (e.g. —CH₂CH₂OH or —CH₂CH₂CH₂OH), —(CH)₂N(R⁵)₂ (e.g. —CH₂CH₂N(CH₃)₂), C₃₋₁₂cycloalkyl

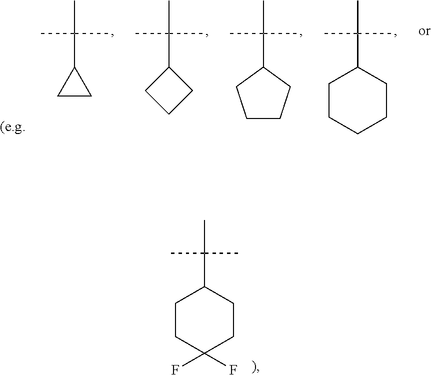

3- to 15-membered heterocyclyl

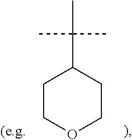

3- to 15-membered heterocyclylC₁₋₈alkyl

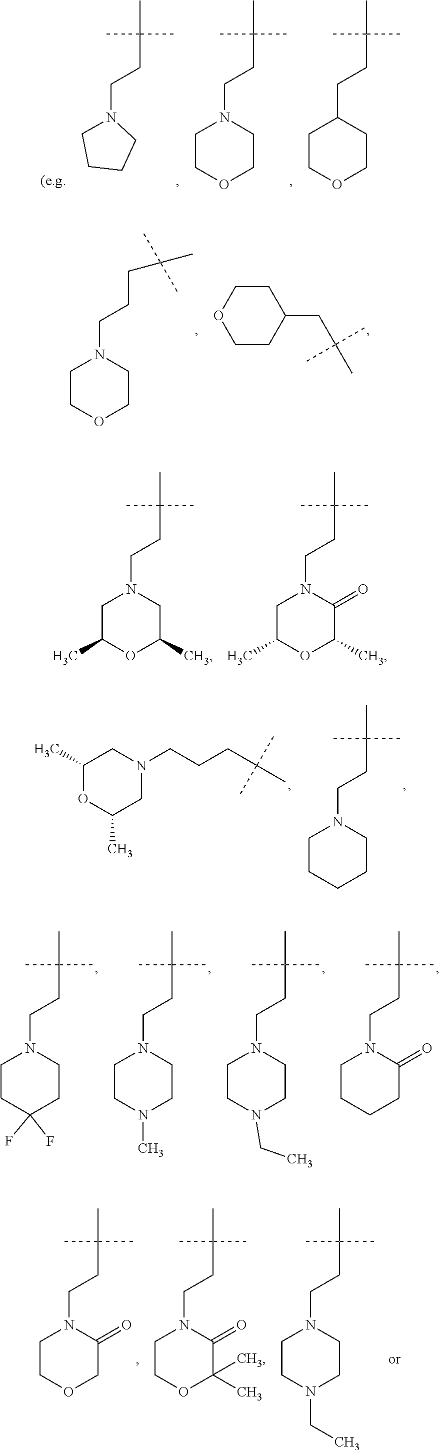

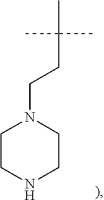

### C₆₋₁₄aryl

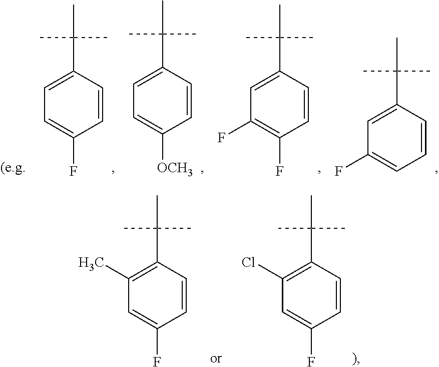

C₆₋₁₄arylC₁₋₈alkyl

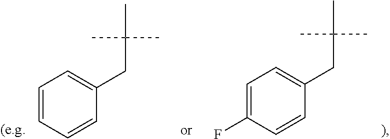

5- to 14-membered heteroaryl

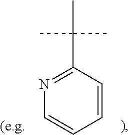

5- to 14-membered heteroarylC₁₋₈alkyl

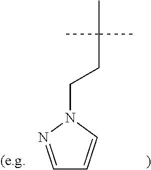

or —(CH₂)ₘS(O)_{P}R⁸

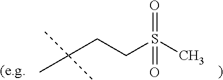

wherein C₃₋₁₂cycloalkyl, 3- to 15-membered heterocyclylC₁₋₈alkyl, C₆₋₁₄aryl and C₆₋₁₄arylC₁₋₈alkyl are optionally substituted with one or more substituents selected from halogen (e.g. Cl, F or Br), oxo (e.g. ═O), C₁₋₈alkyl (e.g. methyl or ethyl) and C₁₋₈alkoxy (e.g. methoxy). In this embodiment, R⁵ is C₁₋₈alkyl (e.g methyl); R⁸ is C₁₋₈alkyl (e.g methyl); m′ is 2 or 3 and ‘p’ is 2.

According to yet another embodiment, specifically provided are compounds of formula (II), in which R³ is hydrogen, C₁₋₈alkyl (e.g. methyl, ethyl, propyl, isopropyl, isobutyl or isopentyl), haloC₁₋₈alkyl (e.g. trifluoroethyl), —(CH₂)ₘOR⁵ (e.g. —CH₂CH₂OCH₃ or —CH₂CH₂CH₂OCH₃), hydroxyC₁₋₈alkyl (e.g. —CH₂CH₂OH or —CH₂CH₂CH₂OH), —(CH)₂N(R⁵)₂ (e.g. —CH₂CH₂N(CH₃)₂), C₃₋₁₂cycloalkyl

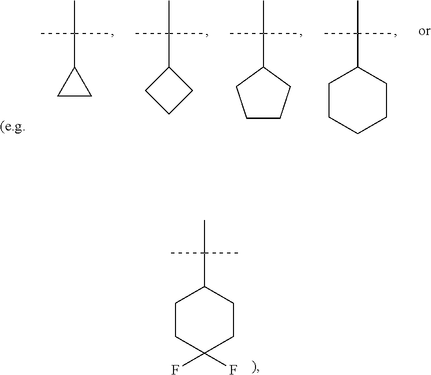

3- to 15-membered heterocyclyl

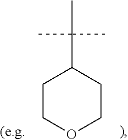

3- to 15-membered heterocyclylC₁₋₈alkyl

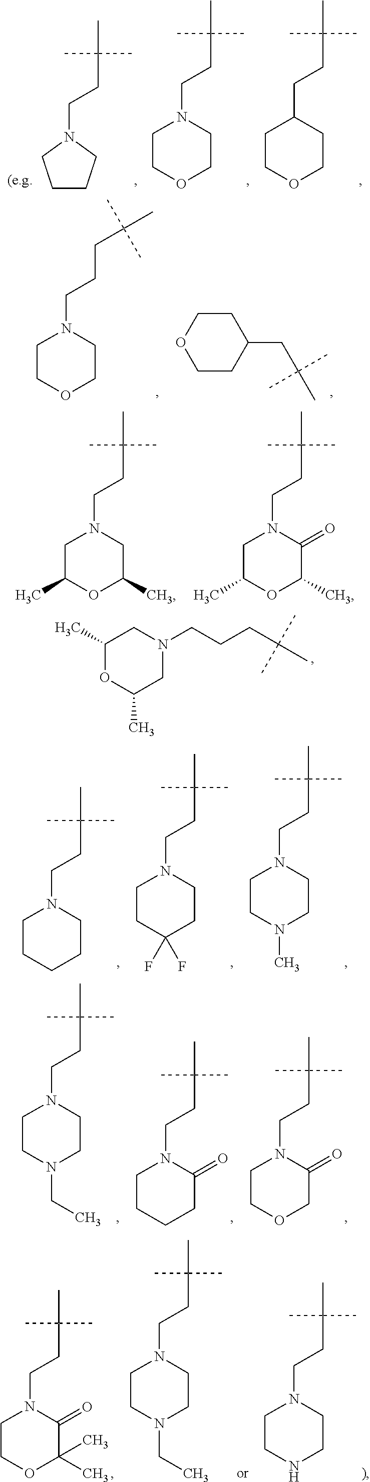

### C₆₋₁₄aryl

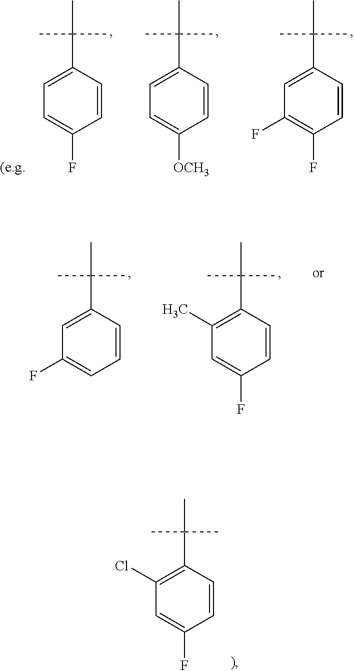

C₆₋₁₄arylC₁₋₈alkyl

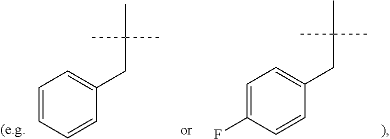

5- to 14-membered heteroaryl

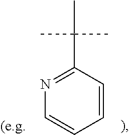

5- to 14-membered heteroarylC₁₋₈alkyl

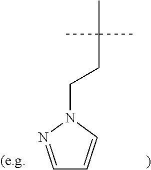

or —(CH₂)ₘS(O)_{P}R⁸

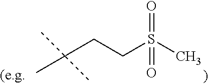

wherein C₃₋₁₂Cycloalkyl, 3- to 15-membered heterocyclylC₁₋₈alkyl, C₆₋₁₄aryl and C₆₋₁₄arylC₁₋₈alkyl are optionally substituted with one or more substituents selected from Cl, F, ═O, methyl, ethyl and methoxy. In this embodiment, R⁵ is methyl; R⁸ is methyl; m′ is 2 or 3 and ‘p’ is 2.

According to yet another embodiment, specifically provided are compounds of formula (II), in which R³ is hydrogen, methyl, ethyl, propyl, isopropyl, isobutyl, isopentyl, trifluoroethyl, —CH₂CH₂OCH₃, —CH₂CH₂CH₂OCH₃, —CH₂CH₂OH, —CH₂CH₂CH₂OH, —CH₂CH₂N(CH₃)₂,

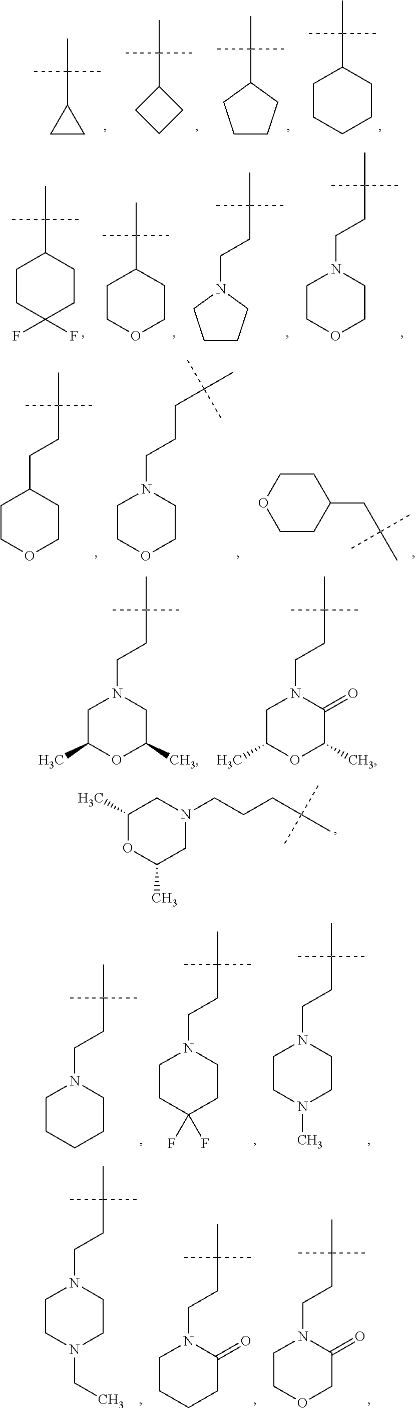

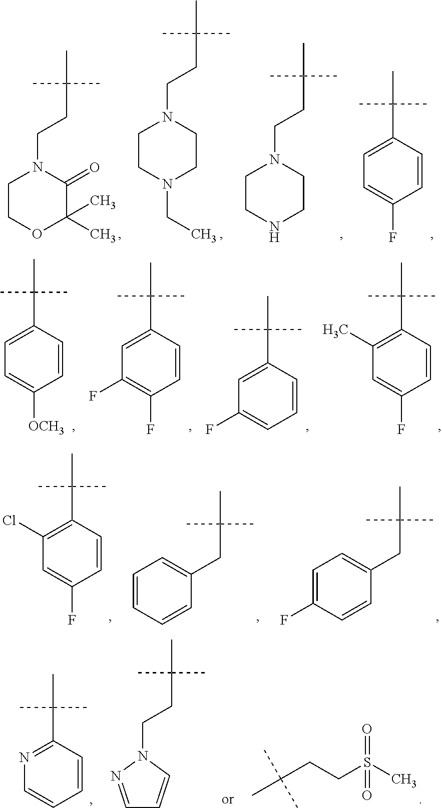

According to yet another embodiment, specifically provided are compounds of formula (II), in which

Z³ is CH or N;

R¹ is F, Cl, NH₂, OH, methyl, methoxy, —OCH₂CH₂OCH₃, CF3, OCF3,

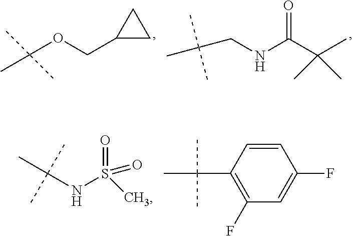

### or 1H-imidazol-1-yl;

R² is hydrogen, methyl, ethyl, isopropyl, isobutyl, trifluoromethyl, difluoromethyl, —CH₂OH, —CH₂OCH₃, —C(O)NH₂),

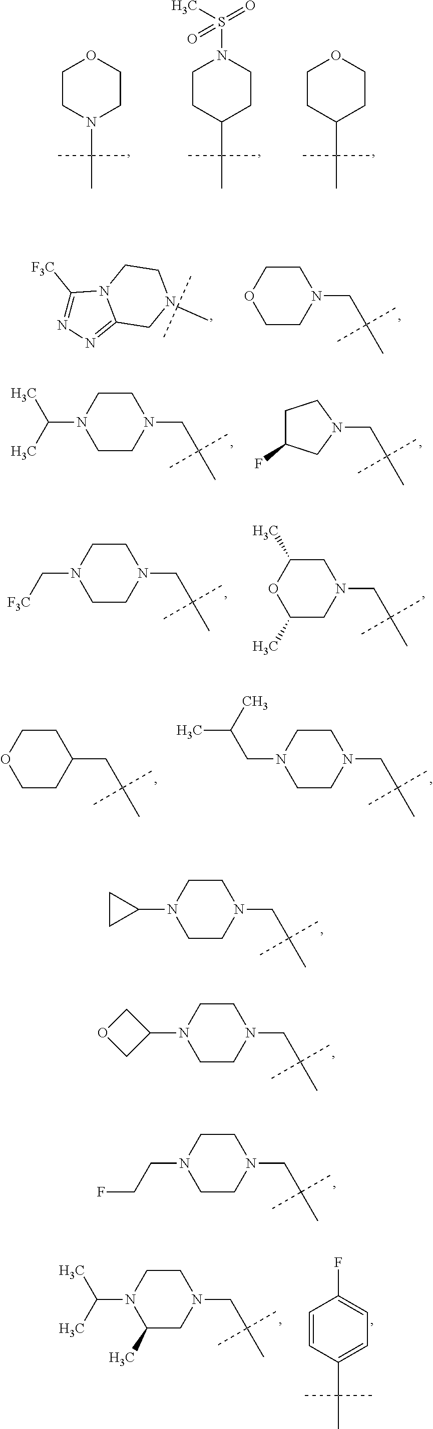

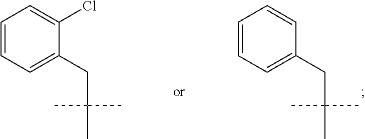

R³ is hydrogen, methyl, ethyl, propyl, isopropyl, isobutyl or isopentyl, trifluoroethyl, —CH₂CH₂OCH₃, —CH₂CH₂CH₂OCH₃, —CH₂CH₂OH, —CH₂CH₂CH₂OH, —CH₂CH₂N(CH₃)₂,

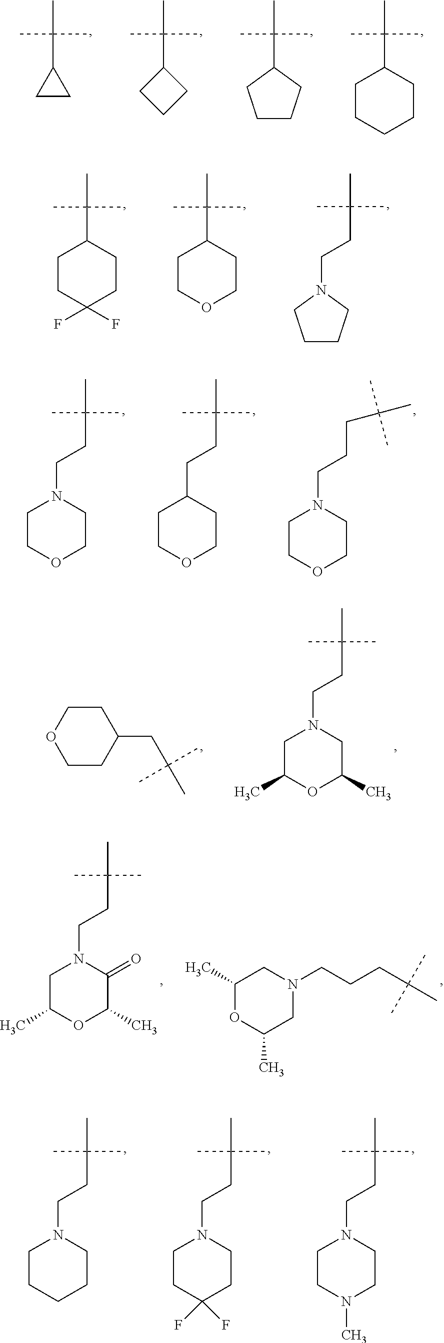

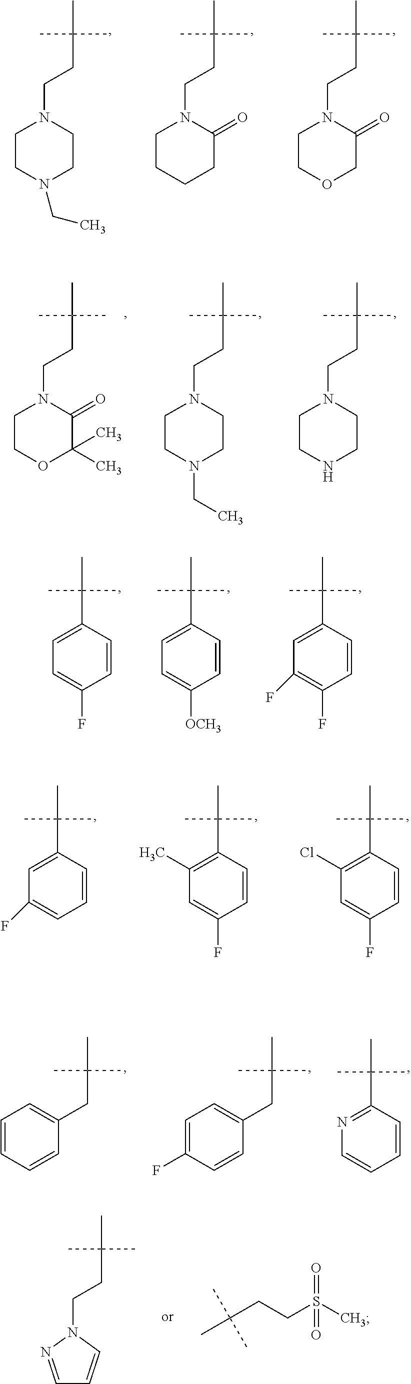

and

‘n’ is 0, 1, 2 or 3.

According to yet another embodiment, specifically provided are compounds of formula (II), in which

Z³ is CH;

R¹ is F, Cl, NH₂, OH, methyl, methoxy, —OCH₂CH₂OCH₃, CF3, OCF3,

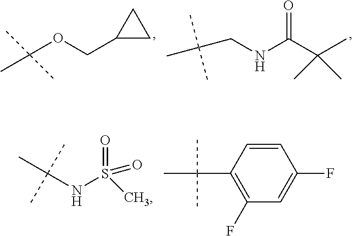

### or 1H-imidazol-1-yl ;

R² is hydrogen, methyl, ethyl, isopropyl, isobutyl, trifluoromethyl, difluoromethyl, —CH₂OH, —CH₂OCH₃, —C(O)NH₂),

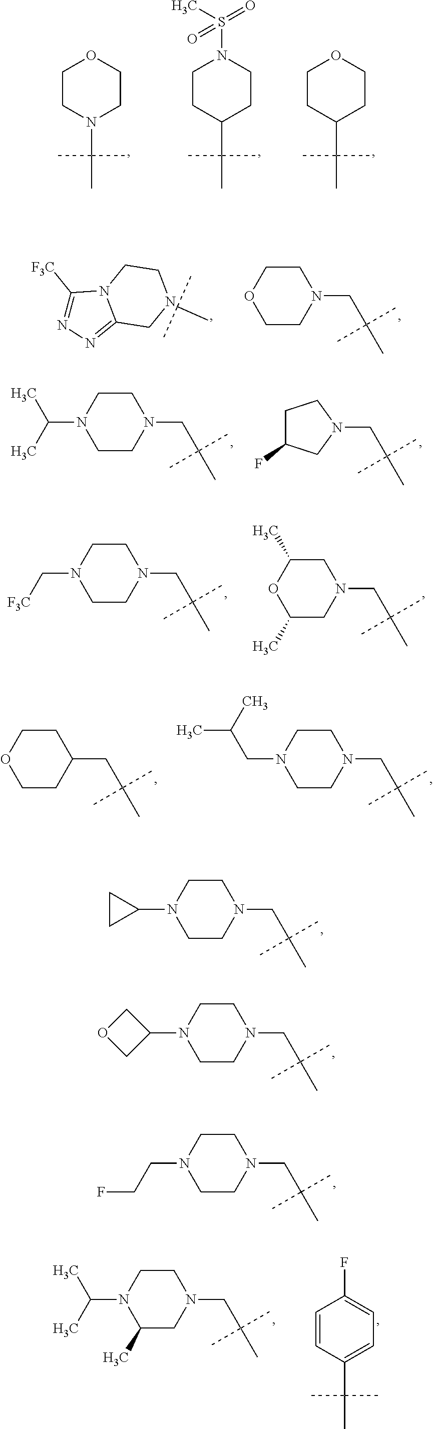

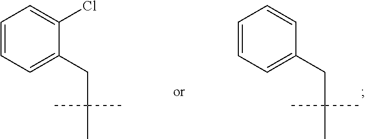

R³ is hydrogen, methyl, ethyl, propyl, isopropyl, isobutyl, isopentyl, trifluoroethyl, —CH₂CH₂OCH₃, —CH₂CH₂CH₂OCH₃, —CH₂CH₂OH, —CH₂CH₂CH₂OH, —CH₂CH₂N(CH₃)₂,

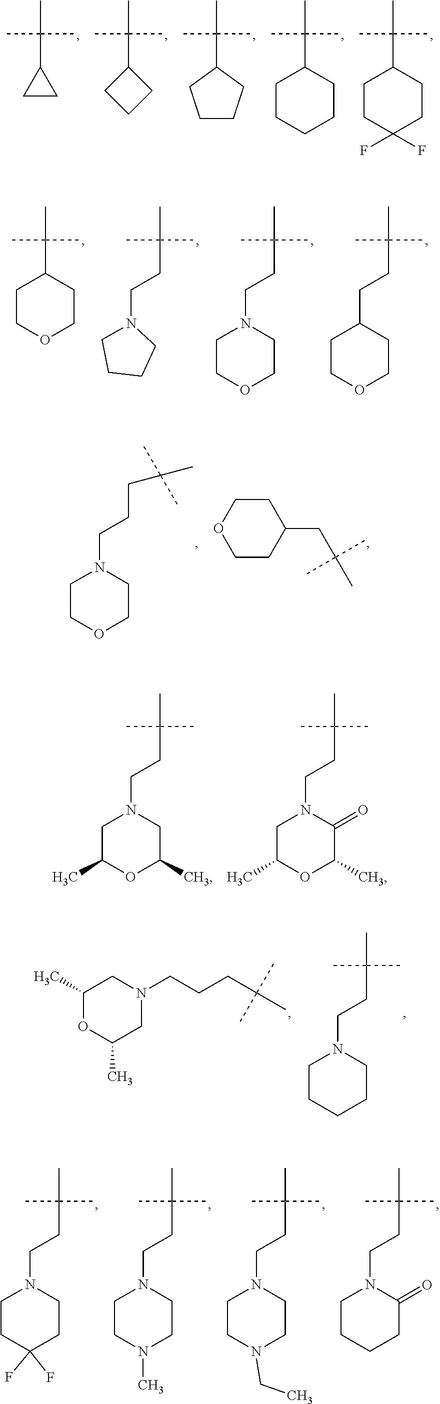

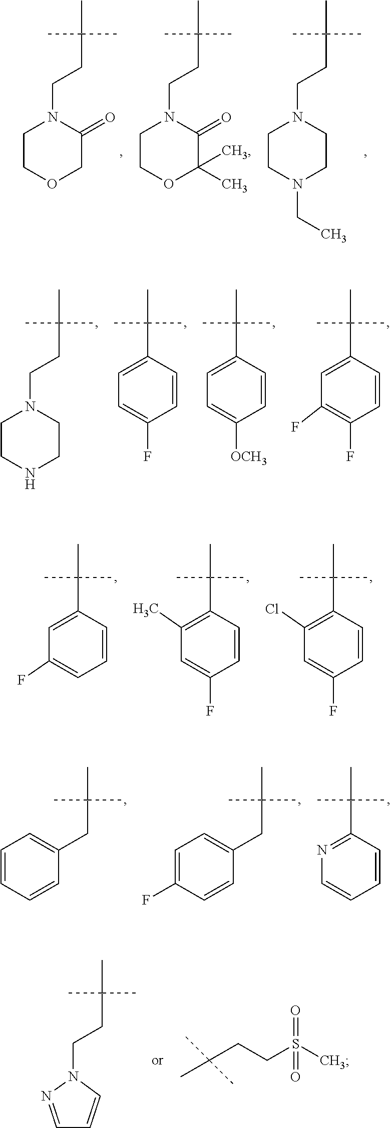

and

‘n’ is 1, 2 or 3.

According to an embodiment, specifically provided are compounds of formula (II) with an IC₅₀ value of less than 1100 nM, preferably, less than 100 nM, more preferably less than 50 nM, with respect to NADPH oxidase inhibitor activity.

It should be understood that the formulas (I) and (II), structurally encompass all geometrical isomers, stereoisomers, enantiomers and diastereomers, N-oxides, and pharmaceutically acceptable salts that may be contemplated from the chemical structure of the genera described herein.

According to an embodiment, the compounds of formula (I) (wherein X is NH) or formula (II) (wherein X is NH), structurally encompass all tautomeric forms whether such tautomer exists in equilibrium or predominantly in one form. Such tautomeric form may be different or the same when the compound is bound to the NADPH oxidase enzyme.

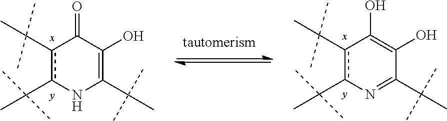

The present invention also provides a pharmaceutical composition that includes at least one compound described herein or a pharmaceutically acceptable salt thereof and at least one pharmaceutically acceptable excipient (such as a pharmaceutically acceptable carrier or diluent). Preferably, the pharmaceutical composition comprises a therapeutically effective amount of at least one compound described herein. The compounds described in the present patent application may be associated with a pharmaceutically acceptable excipient (such as a carrier or a diluent) or be diluted by a carrier, or enclosed within a carrier which can be in the form of a capsule, sachet, paper or other container.

The compounds and pharmaceutical compositions of the present invention are useful for inhibiting the activity of NADPH, which is related to a variety of disease states.

The present invention further provides a method of inhibiting NADPH oxidase in a subject in need thereof by administering to the subject one or more compounds described herein in an amount effective to cause inhibition of NADPH.

### DETAILED DESCRIPTION OF THE INVENTION

### Definitions

The terms “halogen” or “halo” means fluorine (fluoro), chlorine (chloro), bromine (bromo), or iodine (iodo).

The term “alkyl” refers to a straight or branched hydrocarbon chain radical that includes solely carbon and hydrogen atoms in the backbone, containing no unsaturation, having from one to eight carbon atoms (i.e. C₁₋₈alkyl), and which is attached to the rest of the molecule by a single bond. “C₁₋₆ alkyl” is an alkyl group that has from 1 to 6 carbon atoms. Non-limiting examples of alkyl groups include methyl, ethyl, n-propyl, 1-methylethyl (isopropyl), n-butyl, 2-methylpropyl (isobutyl), n-pentyl, 1,1-dimethylethyl (t-butyl), and 2,2-dimethylpropyl.

The term “alkoxy” denotes an alkyl group attached via an oxygen linkage to the rest of the molecule (i.e. C₁₋₈alkoxy). Representative examples of such groups are —OCH₃ and —OC₂ H₅.

The term “alkoxyalkoxy” denotes an alkoxy group attached via an oxygen linkage to the rest of the molecule (i.e. C₁₋₈alkoxy). Example of such alkoxyalkoxy moiety includes, but not limited to, —OCH₂—CH₂OCH₃ and —OCH₂CH₂OC₂ H₅.

The term “haloalkyl” refers to at least one halo group (selected from F, Cl, Br or I), linked to an alkyl group as defined above (i.e. haloC₁₋₈alkyl). Examples of such haloalkyl moiety include, but are not limited to, trifluoromethyl, trifluoroethyl, difluoromethyl and fluoromethyl groups.

The term “haloalkoxy” refers to an alkoxy group substituted with one or more halogen atoms (i.e. haloC₁₋₈alkoxy). Examples of “haloalkoxy” include but are not limited to fluoromethoxy, difluoromethoxy, trifluoromethoxy, 2,2,2-trifluoroethoxy, pentafluoroethoxy, pentachloroethoxy, chloromethoxy, dichlorormethoxy, trichloromethoxy and 1-bromoethoxy.

The term “hydroxyalkyl” refers to an alkyl group as defined above wherein one to three hydrogen atoms on different carbon atoms is/are replaced by hydroxyl groups (i.e. hydroxyC₁₋₈alkyl). Examples of hydroxyalkyl moiety include, but are not limited to —CH₂OH, —C2 H₄OH and —CH(OH)C₂ H₄OH.

The term “cycloalkyl” denotes a non-aromatic mono or multicyclic ring system of 3 to about 12 carbon atoms, for example C₃₋₁₂cycloalkyl, such as cyclopropyl, cyclobutyl, cyclopentyl, and cyclohexyl. Examples of multicyclic cycloalkyl groups include, but are not limited to, perhydronapththyl, adamantyl and norbornyl groups, bridged cyclic groups or spirobicyclic groups, e.g., spiro(4,4)non-2-yl.

The term “cycloalkylalkyl” refers to a cyclic ring-containing radical having 3 to about 8 carbon atoms directly attached to an alkyl group, for example C₃₋₈cycloalkylC₁₋₈alkyl. The cycloalkylalkyl group may be attached to the main structure at any carbon atom in the alkyl group that results in the creation of a stable structure. Non-limiting examples of such groups include cyclopropylmethyl, cyclobutylethyl, and cyclopentylethyl.

The term “aryl” refers to an aromatic radical having 6 to 14 carbon atoms (i.e. C₆₋₁₄aryl), including monocyclic, bicyclic and tricyclic aromatic systems, such as phenyl, naphthyl, tetrahydronapthyl, indanyl, and biphenyl.

The term “arylalkyl” refers to an aryl group as defined above directly bonded to an alkyl group as defined above, i.e. C₆₋₁₄arylC₁₋₈alkyl, such as —CH₂C₆ H₅ and —C₂ H₄C₆ H₅.

The term “heterocyclyl” or “heterocyclic ring” unless otherwise specified refers to substituted or unsubstituted non-aromatic 3- to 15-membered ring radical which consists of carbon atoms and from one to five hetero atoms selected from nitrogen, phosphorus, oxygen and sulfur. The heterocyclic ring radical may be a mono-, bi- or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; also, unless otherwise constrained by the definition the heterocyclic ring or heterocyclyl may optionally contain one or more olefinic bond(s). Examples of such heterocyclic ring radicals include, but are not limited to azepinyl, azetidinyl, benzodioxolyl, benzodioxanyl, chromanyl, dioxolanyl, dioxaphospholanyl, decahydroisoquinolyl, indanyl, indolinyl, isoindolinyl, isochromanyl, isothiazolidinyl, isoxazolidinyl, morpholinyl, oxazolinyl, oxazolidinyl, oxadiazolyl, 2-oxopiperazinyl, 2-oxopiperidinyl, 2-oxopyrrolidinyl, 2-oxoazepinyl, octahydroindolyl, octahydroisoindolyl, perhydroazepinyl, piperazinyl, 4-piperidonyl, pyrrolidinyl, piperidinyl, phenothiazinyl, phenoxazinyl, quinuclidinyl, tetrahydroisquinolyl, tetrahydrofuryl, tetrahydropyranyl, thiazolinyl, thiazolidinyl, thiamorpholinyl, thiamorpholinyl sulfoxide and thiamorpholinyl sulfone. The heterocyclic ring radical may be attached to the main structure at any heteroatom or carbon atom that results in the creation of a stable structure.

The term “heterocyclylalkyl” refers to a heterocyclic ring radical directly bonded to an alkyl group (i.e. heterocyclylC₁₋₈alkyl). The heterocyclylalkyl radical may be attached to the main structure at any carbon atom in the alkyl group that results in the creation of a stable structure.

The term “heteroaryl” unless otherwise specified refers to substituted or unsubstituted 5- to 14-membered aromatic heterocyclic ring radical with one or more heteroatom(s) independently selected from N, O or S. The heteroaryl may be a mono-, bi- or tricyclic ring system. The heteroaryl ring radical may be attached to the main structure at any heteroatom or carbon atom that results in the creation of a stable structure. Examples of such heteroaryl ring radicals include, but are not limited to oxazolyl, isoxazolyl, imidazolyl, furyl, indolyl, isoindolyl, pyrrolyl, pyrazolyl, triazolyl, triazinyl, tetrazoyl, thienyl, thiazolyl, isothiazolyl, pyridyl, pyridinyl, pyrimidinyl, pyrazinyl, pyridazinyl, benzofuranyl, benzothiazolyl, benzoxazolyl, benzimidazolyl, benzothienyl, benzopyranyl, carbazolyl, quinolinyl, isoquinolinyl, quinazolinyl, cinnolinyl, naphthyridinyl, pteridinyl, purinyl, quinoxalinyl, quinolyl, isoquinolyl, thiadiazolyl, indazolyl, indolizinyl, acridinyl, phenazinyl and phthalazinyl.

The term “heteroarylalkyl” refers to a heteroaryl ring radical directly bonded to an alkyl group (i.e. heterarylC₁₋₈alkyl). The heteroarylalkyl radical may be attached to the main structure at any carbon atom in the alkyl group that results in the creation of a stable structure.

The term “pharmaceutically acceptable salt” includes salts prepared from pharmaceutically acceptable bases or acids including inorganic or organic bases and inorganic or organic acids. Examples of such salts include, but are not limited to, acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate, diphosphate, polygalacturonate, salicylate, stearate, sulfate, subacetate, succinate, tannate, tartrate, teoclate, tosylate, triethiodide and valerate. Examples of salts derived from inorganic bases include, but are not limited to, aluminum, ammonium, calcium, copper, ferric, ferrous, lithium, magnesium, manganic, mangamous, potassium, sodium, and zinc.

The term “treating” or “treatment” of a state, disorder or condition includes: (a) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (b) inhibiting the state, disorder or condition, i.e., arresting or reducing the development of the disease or at least one clinical or subclinical symptom thereof; or (c) relieving the disease, i.e., causing regression of the state, disorder or condition or at least one of its clinical or subclinical symptoms.

The term “subject” includes mammals (especially humans) and other animals, such as domestic animals (e.g., household pets including cats and dogs) and non-domestic animals (such as wildlife).

A “therapeutically effective amount” means the amount of a compound that, when administered to a subject for treating a state, disorder or condition, is sufficient to effect such treatment. The “therapeutically effective amount” will vary depending on the compound, the disease and its severity and the age, weight, physical condition and responsiveness of the subject to be treated.

The compound described in the present patent application may form salts. Non-limiting examples of pharmaceutically acceptable salts forming part of this patent application include salts derived from inorganic bases salts of organic bases salts of chiral bases, salts of natural amino acids and salts of non-natural amino acids.

Certain compounds of present patent application are capable of existing in stereoisomeric forms (e.g. diastereomers and enantiomers). With respect to the overall compounds described by the general formula (I) the present patent application extends to these stereoisomeric forms and to mixtures thereof. To the extent prior art teaches synthesis or separation of particular stereoisomers, the different stereoisomeric forms of the present patent application may be separated from one another by the method known in the art, or a given isomer may be obtained by stereospecific or asymmetric synthesis. Tautomeric forms and mixtures of compounds described herein are also contemplated. It is also to be understood that compounds of the invention may exist in solvated forms (such as hydrates) as well as unsolvated forms, and that the invention encompasses all such forms.

### Pharmaceutical Compositions

The compounds of the invention are typically administered in the form of a pharmaceutical composition. Such compositions can be prepared using procedures well known in the pharmaceutical art and comprise at least one compound of the invention. The pharmaceutical composition of the present patent application comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents, solvents and the like.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters, and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, capsules, tablets, solutions, suspensions, injectables or products for topical application. Further, the pharmaceutical composition of the present invention may be formulated so as to provide desired release profile.

Administration of the compounds of the invention, in pure form or in an appropriate pharmaceutical composition, can be carried out using any of the accepted routes of administration of pharmaceutical compositions. The route of administration may be any route which effectively transports the active compound of the patent application to the appropriate or desired site of action. Suitable routes of administration include, but are not limited to, oral, nasal, buccal, dermal, intradermal, transdermal, parenteral, rectal, subcutaneous, intravenous, intraurethral, intramuscular or topical.

Solid oral formulations include, but are not limited to, tablets, capsules (soft or hard gelatin), dragees (containing the active ingredient in powder or pellet form), troches and lozenges.

Liquid formulations include, but are not limited to, syrups, emulsions, and sterile injectable liquids, such as suspensions or solutions.

Topical dosage forms of the compounds include ointments, pastes, creams, lotions, powders, solutions, eye or ear drops, impregnated dressings, and may contain appropriate conventional additives such as preservatives, solvents to assist drug penetration.

The pharmaceutical compositions of the present patent application may be prepared by conventional techniques, e.g., as described in Remington: The Science and Practice of Pharmacy, 20^{th} Ed., 2003 (Lippincott Williams & Wilkins).

Suitable doses of the compounds for use in treating the diseases and disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms, and suitable pharmaceutical excipients can also be well used and adjusted by those skilled in the art. All changes and modifications are envisioned within the scope of the present patent application.

### Methods of Treatment

Compounds of the present patent application inhibit the activity of NADPH oxidase (nicotinamide adenine dinucleotide phosphate oxidase) i.e., they prevent or suppress the action of NADPH oxidase, and/or elicit NADPH oxidase modulating effect, thereby reducing the generation of reactive oxygen species (ROS). Compounds of the present invention are thus useful in the treatment of numerous diseases and disorders mediated by ROS derived from NADPH oxidase.

Compounds of the present patent application are thus expected to be useful in the treatment of pain, inflammatory disorders, bone disorders, autoimmune diseases, cardiovascular disorders, endocrine disorders, respiratory disorders, metabolism disorders, skin disorders, neuroinflammatory and/or neurodegenerative disorders, kidney diseases, reproduction disorders, endocrine disorders, diseases affecting the eye and/or the lens and/or conditions affecting the inner ear, liver diseases, cancers, allergic disorders, traumatisms, septic, hemorrhagic and anaphylactic shock, diseases or disorders of the gastrointestinal system, angiogenesis, angiogenesis-dependent conditions, as well as lung infections, acute lung injury, pulmonary arterial hypertension, obstructive lung disorders, fibrotic lung disease, and cancer.

The term “pain” includes, but not limited to, nociceptive pain, dental pain, cardiac pain arising from an ischemic myocardium, pain due to migraine, acute pain, chronic pain, neuropathic pain, post-operative pain, pain due to neuralgia (e.g., post-herpetic neuralgia or trigeminal neuralgia), pain due to diabetic neuropathy, low back and neck pain, dysmenorrhea, headache, migraine (acute and prophylactic treatment), toothache, sprains and strains, acute, subacute and chronic musculoskeletal pain syndromes such as bursitis, burns, injuries, pain following surgical (post-operative pain) and dental procedures as well as the preemptive treatment of surgical pain, cancer pain and inflammatory pain conditions such as myositis, synovitis, acute gout and ankylosing spondylitis and arthritis (including rheumatoid arthritis, juvenile rheumatoid arthritis and osteoarthritis).

The term “inflammatory disorder” includes, but not limited to, inflammatory bowel disease, sepsis, septic shock, adult respiratory distress syndrome, pancreatitis, shock induced by trauma, asthma, bronchial asthma, allergic rhinitis, rheumatoid arthritis, chronic rheumatoid arthritis, arteriosclerosis, intracerebral hemorrhage, cerebral infarction, heart failure, myocardial infarction, psoriasis, cystic fibrosis, liver fibrosis, stroke, acute bronchitis, chronic bronchitis, acute bronchiolitis, chronic bronchiolitis, osteoarthritis, gout, myelitis, ankylosing spondylitis, Reuter syndrome, psoriatic arthritis, spondylarthritis, juvenile arthritis or juvenile ankylosing spondylitis, reactive arthritis, infectious arthritis or arthritis after infection, gonococcal arthritis, syphilitic arthritis, Lyme disease, arthritis induced by “angiitis syndrome,” polyarteritis nodosa, anaphylactic angiitis, Luegenec granulomatosis, rheumatoid polymyalgia, articular cell rheumatism, calcium crystal deposition arthritis, pseudogout, non-arthritic rheumatism, bursitis, tendosynovitis, epicondyle inflammation (tennis elbow), carpal tunnel syndrome, disorders by repetitive use (typing), mixed form of arthritis, neuropathic arthropathy, hemorrhagic arthritis, vascular peliosis, hypertrophic osteoarthropathy, multicentric reticulohistiocytosis, arthritis induced by specific diseases, blood pigmentation, sickle cell disease and other hemoglobin abnormality, hyperlipoproteinemia, dysgammaglobulinemia, hyperparathyroidism, acromegaly, familial Mediterranean fever, Bechet's disease, systemic autoimmune disease erythematosus, multiple sclerosis and Crohn's disease or diseases like relapsing polychondritis or chronic inflammatory bowel diseases (IBD).

The term “autoimmune diseases” will be understood by those skilled in the art to refer to a condition that occurs when the immune system mistakenly attacks and destroys healthy body tissue. An autoimmune disorder may result in the destruction of one or more types of body tissue, abnormal growth of an organ, and changes in organ function. An autoimmune disorder may affect one or more organ or tissue types which include, but are not limited to, blood vessels, connective tissues, endocrine glands such as the thyroid or pancreas, joints, muscles, red blood cells, and skin. Examples of autoimmune (or autoimmune-related) disorders include multiple sclerosis, arthritis, scleroderma, rheumatoid arthritis, psoriasis, Crohn's disease, gastrointestinal disorder, inflammatory bowel disease, irritable bowel syndrome, colitis, ulcerative colitis, Sjorgen's syndrome, atopic dermatitis, optic neuritis, respiratory disorder, chronic obstructive pulmonary disease (COPD), asthma, type I diabetes, neuromyelitis optica, Myasthenia Gavis, uveitis, Guillain- Barre syndrome, psoriatic arthritis, Gaves' disease, allergy, osteoarthritis, Kawasaki disease, mucosal leishmaniasis, Hashimoto's thyroiditis, Pernicious anemia, Addison's disease, Systemic lupus erythematosus, Dermatomyositis, Sjogren syndrome, Lupus erythematosus, Myasthenia gravis, Reactive arthritis, Celiac disease - sprue (gluten-sensitive enteropathy), Graves's disease, thymopoiesis and Lupus.

The term “bone disorder” includes, but not limited to, osteoporosis, osteosclerosis, periodontitis, and hyperparathyroidism.

The term “cardiovascular disorder” comprises atherosclerosis, especially diseases or disorders associated with endothelial dysfunction including but not limited to hypertension, cardiovascular complications of Type I or Type II diabetes, intimal hyperplasia, coronary heart disease, cerebral, coronary or arterial vasospasm, endothelial dysfunction, heart failure including congestive heart failure, peripheral artery disease, restenosis, trauma caused by a stent, stroke, ischemic attack, vascular complications such as after organ transplantation, myocardial infarction, hypertension, formation of atherosclerotic plaques, platelet aggregation, angina pectoris, aneurysm, aortic dissection, ischemic heart disease, cardiac hypertrophy, pulmonary embolus, thrombotic events including deep vein thrombosis, injury caused after ischemia by restoration of blood flow or oxygen delivery as in organ transplantation, open heart surgery, angioplasty, hemorrhagic shock, angioplasty of ischemic organs including heart, brain, liver, kidney, retina and bowel.

The term “respiratory disorder” includes, but not limited to, asthma, cough, bronchial asthma, bronchitis, allergic rhinitis, acute respiratory distress syndrome, cystic fibrosis, lung viral infection (influenza), pulmonary hypertension, idiopathic pulmonary fibrosis, chronic obstructive pulmonary diseases (COPD) and COPD exacerbation.

The “allergic disorder” includes, but not limited to, cough, hay fever and asthma. The “metabolism disorder” includes, but not limited to, obesity, metabolic syndrome and Type II diabetes. The “skin disorder” includes, but not limited to, psoriasis, eczema, dermatitis, wound healing and scar formation.

The “neurodegenerative disorder” comprises a disease or a state characterized by a central nervous system (CNS) degeneration or alteration, especially at the level of the neurons such as Alzheimer's disease, Parkinson's disease, Huntington's disease, amyotrophic lateral sclerosis, epilepsy and muscular dystrophy. It further comprises neuro-inflammatory and demyelinating states or dis eas es such as leukoencephalopathies, and leukodystrophies. The term “demyelinating” is referring to a state or a disease of the CNS comprising the degradation of the myelin around the axons. In the context of the invention, the term demyelinating disease is intended to comprise conditions which comprise a process that demyelinate cells such as multiple sclerosis, progressive multifocal leukoencephalopathy (PML), myelopathies, any neuroinflammatory condition involving autoreactive leukocyte within the CNS, congenital metabolic disorder, a neuropathy with abnormal myelination, drug induced demyelination, radiation induced demyelination, a hereditary demyelinating condition, a prion induced demyelinating condition, encephalitis induced demyelination or a spinal cord injury. Preferably, the condition is multiple sclerosis.

The “kidney disease” includes, but not limited to, diabetic nephropathy, renal failure, glomerulonephritis, nephrotoxicity of aminoglycosides and platinum compounds and hyperactive bladder. In a particular embodiment, the term according to the invention includes chronic kidney diseases or disorders. The “reproduction disorder” includes, but not limited to, erectile dysfunction, fertility disorders, prostatic hypertrophy and benign prostatic hypertrophy.

The “disease affecting the eye and/or the lens” includes, but not limited to, cataract including diabetic cataract, re-opacification of the lens post cataract surgery, diabetic and other forms of retinopathy. The “conditions affecting the inner ear” includes presbyacusis, tinnitus, Meniere's disease and other balance problems, utriculolithiasis, vestibular migraine, and noise induced hearing loss and drug induced hearing loss (ototoxicity).

The term “cancer” includes, but not limited to, carcinoma (e.g., fibrosarcoma, myxosarcoma, liposarcomachondros arcoma, osteogenic sarcoma, chordoma, angiosarcoma, endothelium sarcoma, lymphangiosarcoma, lymphangioendothelioma, periosteoma, mesothelioma, Ewing's tumor, leiomyosarcoma, rhabdomyosarcoma, colon carcinoma, pancreatic cancer, breast cancer, lung cancer, non-small cell lung cancer, prostate cancer, ovarian cancer, renal cancer, prostatic carcinoma, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma or hepatocellular carcinoma.

The term “liver diseases” includes, but not limited to, hepatitis, liver fibrosis, alcoholic liver disease, fatty liver disease, non-alcoholic fatty liver disease (NAFLD), non-alcoholic steatohepatitis (NASH), Primary biliary cirrhosis or cirrhosis.

Compounds of the present application are useful in the treatment of diseases or disorder mediated by ROS derived from NADPH oxidases.

Compounds of the present patent application are useful in the treatment of pain, inflammatory disorders, bone disorders, cardiovascular disorders, endocrine disorders, respiratory disorders, metabolism disorders, skin disorders, neuroinflammatory and/or neurodegenerative disorders, kidney diseases, reproduction disorders, endocrine disorders, diseases affecting the eye and/or the lens and/or conditions affecting the inner ear, liver diseases, cancers, allergic disorders, traumatisms, septic, hemorrhagic and anaphylactic shock, diseases or disorders of the gastrointestinal system, angiogenesis, angiogenesis-dependent conditions, as well as lung infections, acute lung injury, pulmonary arterial hypertension, obstructive lung disorders, fibrotic diseases, fibrotic lung disease and cancer.

In an embodiment, the compounds of the present patent application are useful in the treatment of pain, particularly, nociceptive pain, dental pain, cardiac pain arising from an ischemic myocardium, pain due to migraine, acute pain, chronic pain, neuropathic pain, post-operative pain, pain due to neuralgia (e.g., post-herpetic neuralgia or trigeminal neuralgia), pain due to diabetic neuropathy, dental pain, low back and neck pain, dysmenorrhea, headache, migraine (acute and prophylactic treatment), toothache, sprains and strains, acute, subacute and chronic musculoskeletal pain syndromes such as bursitis, burns, injuries, pain following surgical (post-operative pain) and dental procedures as well as the preemptive treatment of surgical pain, cancer pain and inflammatory pain conditions such as myositis, synovitis, acute gout and ankylosing spondylitis and arthritis (including rheumatoid arthritis, juvenile rheumatoid arthritis and osteoarthritis).

In another embodiment, the compounds of the present patent application are useful in the treatment of pain, inflammatory disorders, autoimmune diseases, cardiovascular disorders, respiratory disorders, metabolism disorders, skin disorders, kidney diseases, liver diseases or allergic disorders.

In another embodiment, the compounds of the present patent application are useful in the treatment of pain or inflammation.

In yet another embodiment, the compounds of the present patent application are useful in the treatment of pain.

In yet another embodiment, the compounds of the present patent application are useful in the treatment of chronic pain, acute pain or neuropathic pain.

In yet another embodiment, the compounds of the present patent application are useful in the treatment of inflammatory pain conditions.

In yet another embodiment, the compounds of the present patent application are useful in the treatment of inflammatory disorders.

In yet another embodiment, the compounds of the present patent application are useful in the treatment of metabolic disorder.

In yet another embodiment, the compounds of the present patent application are useful in the treatment of diabetes.

In yet another embodiment, the compounds of the present patent application are useful in the treatment of Type II diabetes.

In yet another embodiment, the compounds of the present patent application are useful in the treatment of respiratory disorder.

In yet another embodiment, the compounds of the present patent application are useful in the treatment of cystic fibrosis, cough, asthma, idiopathic pulmonary fibrosis, chronic obstructive pulmonary diseases (COPD) or COPD exacerbation.

In yet another embodiment, the compounds of the present patent application are useful in the treatment of cystic fibrosis or idiopathic pulmonary fibrosis.

In yet another embodiment, the compounds of the present patent application are useful in the treatment of idiopathic pulmonary fibrosis.

In yet another embodiment, the compounds of the present patent application are useful in the treatment of allergic disorders.

In yet another embodiment, the compounds of the present patent application are useful in the treatment of asthma.

In yet another embodiment, the compounds of the present patent application are useful in the treatment of cough.

In yet another embodiment, the compounds of the present patent application are useful in the treatment of autoimmune diseases.

In yet another embodiment, the compounds of the present patent application are useful in the treatment of scleroderma.

In yet another embodiment, the compounds of the present patent application are useful in the treatment of kidney disorder.

In yet another embodiment, the compounds of the present patent application are useful in the treatment of diabetic nephropathy.

In yet another embodiment, the compounds of the present patent application are useful in the treatment of pain due to diabetic nephropathy.

In yet another embodiment, the compounds of the present patent application are useful in the treatment of bone disorder.

In yet another embodiment, the compounds of the present patent application are useful in the treatment of osteoporosis.

In yet another embodiment, the compounds of the present patent application are useful in the treatment of disease or disease conditions such as pain, diabetes, cystic fibrosis osteoporosis, asthma, cough, chronic obstructive pulmonary diseases, COPD exacerbation, non-small cell lung cancer, breast cancer, prostate cancer, non-alcoholic fatty liver disease, non-alcoholic steatohepatitis, Primary biliary cirrhosis or cirrhosis.

In yet another embodiment, the compounds of the present patent application are useful in the treatment of cystic fibrosis, cough, asthma, idiopathic pulmonary fibrosis, chronic obstructive pulmonary diseases or COPD exacerbation.

In yet another embodiment, the compounds of the present patent application are useful in the treatment of non-alcoholic fatty liver disease, non-alcoholic steatohepatitis, Primary biliary cirrhosis or cirrhosis.

In yet another embodiment, the compounds of the present patent application are useful in the treatment of non-alcoholic fatty liver disease.

In yet another embodiment, the compounds of the present patent application are useful in the treatment of non-alcoholic steatohepatitis.

In yet another embodiment, the compounds of the present patent application are useful in the treatment of Primary biliary cirrhosis.

In yet another embodiment, the compounds of the present patent application are useful in the treatment of cirrhosis.

In yet another embodiment, the compounds of the present patent application are useful in the treatment of non-small cell lung cancer, breast cancer or prostate cancer.

In yet another embodiment, the compounds of the present patent application are useful in the treatment of lung cancer.

In yet another embodiment, the compounds of the present patent application are useful in the treatment of non-small cell lung cancer.

In yet another embodiment, the compounds of the present patent application are useful in the treatment of breast cancer.

In yet another embodiment, the compounds of the present patent application are useful in the treatment of prostate cancer.

The present patent application relates to the use of the compounds in the preparation of a medicament for the treatment of diseases mediated by ROS derived from NADPH oxidases.

Compounds of the present patent application are indicated both in the therapeutic and/or prophylactic treatment of the above-mentioned conditions. For the above-mentioned therapeutic uses the dosage administered will, of course, vary with the compound employed, the mode of administration, the treatment desired and the disorder indicated. The daily dosage of the compound of the invention may be in the range from 0.05 mg/kg to 100 mg/kg.

### General Methods of Preparation

The compounds described herein, including compounds of general formula (I), (II) and specific examples are prepared using techniques known to one skilled in the art through the reaction sequences depicted in schemes 1-20 as well as by other methods. Furthermore, in the following synthetic schemes, where specific acids, bases, reagents, coupling agents, solvents, etc. are mentioned, it is understood that other suitable acids, bases, reagents, coupling agents etc. may be used and are included within the scope of the present invention. The compounds obtained by using the general reaction sequences may be of insufficient purity. These compounds can be purified by using any of the methods for purification of organic compounds known to persons skilled in the art, for example, crystallization or silica gel or alumina column chromatography using different solvents in suitable ratios.

A general approach for the synthesis of pyrazolo[3,4-b]pyridinone of general formula (IIa) [wherein Z₃, R, R¹, R², R³ and ‘n’ are as defined with respect to a compound of formula (II)] is depicted in synthetic scheme 1.

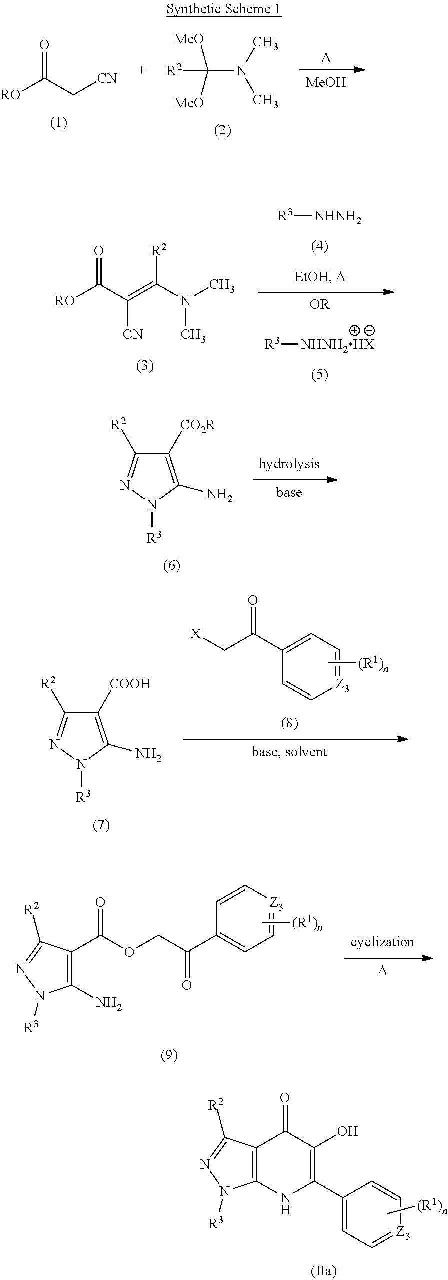

The reaction of alkyl cyanoacetate of the formula (1) with an appropriate acetal of the formula (2) in suitable solvent at elevated temperature affords the corresponding enamine of the formula (3). In an embodiment the reaction may be carried out in the presence of suitable solvent. In an embodiment the suitable solvent may be selected from ethanol, methanol and DMF. In an embodiment the appropriate acetal compound of formula (2) may be selected from N,N′-dimethyl formamide dimethyl acetal and N,N′-dimethyl acetamide dimethyl acetal. In an embodiment the reaction may be carried out in elevated temperature. In an embodiment the elevated temperature may be in the range 50° C. to 150° C. The intermolecular cyclization of enamine of formula (3) with suitably substituted hydrazine of the formula (4) in suitable solvent affords amino pyrazole ester of the formula (6). In an embodiment the reaction may be carried out in the presence of suitable solvent. In an embodiment the suitable solvent is ethanol. In an embodiment the suitably substituted hydrazine is methyl hydrazine. Alternatively amino pyrazole ester of the formula (6) can be prepared by intermolecular cyclization of enamine of the formula (3) with suitable substituted hydrazine salts of the formula (5) in the presence of suitable base. In an embodiment the reaction may be carried out in the presence of a suitable base. In an embodiment the suitable base may be selected from N,N-diisopropylethylamine, triethylamine, sodium hydroxide and potassium hydroxide. In an embodiment the reaction may be carried out in the presence of suitable solvent. In an embodiment the suitable solvent may be selected from dry ethanol and dry methanol. In an embodiment the suitable solvent is dry ethanol. In an embodiment the suitably substituted hydrazine salt is methyl hydrazine sulfate. The ester hydrolysis of amino pyrazole ester of the formula (6) using a suitable base in a mixture of suitable solvent affords the corresponding amino pyrazole carboxylic acid of the formula (7). In an embodiment the reaction may be carried out in the presence of a suitable base. In an embodiment the suitable base may be potassium hydroxide or sodium hydroxide. In an embodiment the reaction may be carried out in the presence of a mixture of suitable solvent. In an embodiment the mixture of suitable solvent is water and ethanol or water and methanol. In an embodiment a mixture of suitable solvent is in the appropriate proportion. In an embodiment the appropriate proportion is 1:3. The coupling reaction of amino pyrazole carboxylic acid of the formula (7) with appropriately substituted phenacyl halide of the formula (8) using suitable base in the presence of suitable solvent gives compound of the formula (9). In an embodiment the reaction may be carried out in the presence of a suitable base. In an embodiment the suitable base may be potassium fluoride. In an embodiment the reaction may be carried out in the presence of suitable solvent. In an embodiment the suitable solvent may be N,N′-dimethyl formamide. The intramolecular cyclization of compound of formula (9) using a suitable dehydrating agent furnishes pyrazolo[3,4-b]pyridinone compound of general formula (IIa). In an embodiment the suitable dehydrating agent may be selected from polyphosphoric acid, phosphorous pentoxide, zinc chloride and sulfuric acid.

Another approach for synthesis pyrazolo[3,4-b]pyridinone compound of general formula (IIa) [wherein Z₃, R, R¹, R², R³ and ‘n’ are as defined with respect to a compound of formula (II)] is depicted in synthetic scheme 2.

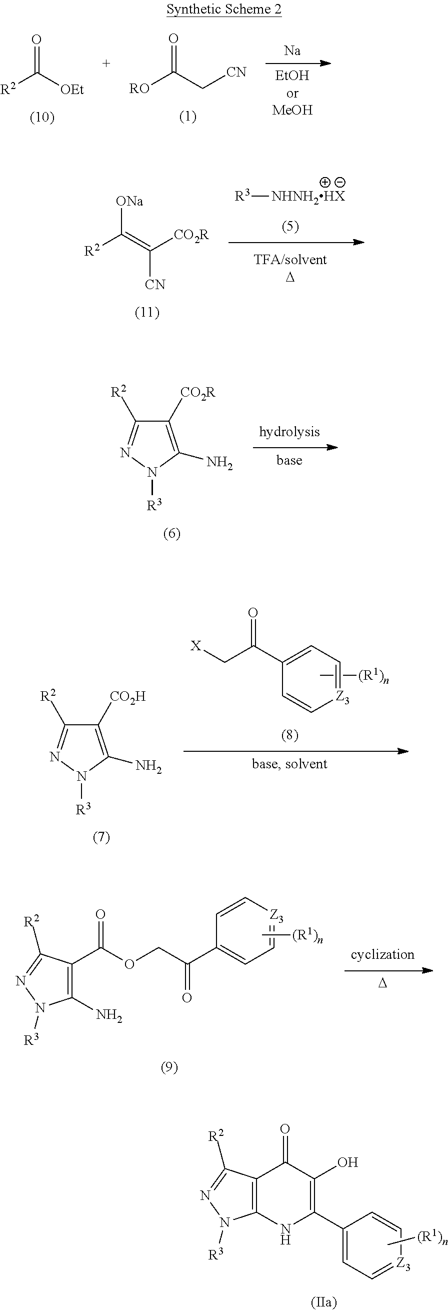

The condensation of appropriately substituted ester of formula (10) and alkyl cyanoacetate of the formula (1) using suitable base affords the corresponding sodium salt of unsaturated hydroxy ester of the formula (11). In an embodiment the reaction may be carried out in the presence of a suitable base. In an embodiment the suitable base is sodium ethoxide or sodium methoxide. The intermolecular cyclization of compound of the formula (11) with suitably substituted hydrazine salt of the formula (5) in the presence of trifluoroacetic acid and suitable solvent affords amino pyrazole ester of the formula (6). In an embodiment the suitably substituted hydrazine salt is methyl hydrazine sulfate. In an embodiment the reaction may be carried out in the presence of suitable solvent. In an embodiment the suitable solvent may diethyl carbonate or dimethyl carbonate. The ester hydrolysis of amino pyrazole ester of the formula (6) using a suitable base in a mixture of suitable solvent affords the corresponding amino pyrazole carboxylic acid of the formula (7). In an embodiment the reaction may be carried out in the presence of a suitable base. In an embodiment the suitable base is sodium hydroxide or potassium hydroxide. In an embodiment the reaction may be carried out in the presence of a mixture of suitable solvent. In an embodiment the mixture of suitable solvent is water and ethanol or water and methanol. In an embodiment a mixture of suitable solvent is in the appropriate proportion. In an embodiment the appropriate proportion is 1:3. The coupling reaction of amino pyrazole carboxylic acid of the formula (7) with appropriately substituted phenacyl halide of the formula (8) in suitable solvent using suitable base affords compound of formula (9). In an embodiment the reaction may be carried out in the presence of a suitable base. In an embodiment the suitable base may be potassium fluoride. In an embodiment the reaction may be carried out in the presence of suitable solvent. In an embodiment the suitable solvent may N,N′-dimethyl formamide. The intramolecular cyclization of compound of the formula (9) using a suitable dehydrating agent gives compound of general formula (IIa). In an embodiment the suitable dehydrating agent may be selected from polyphosphoric acid, phosphorous pentoxide, zinc chloride and sulfuric acid.

Another approach for synthesis pyrazolo[3,4-b]pyridinone compound of general formula (IIa) is depicted in synthetic scheme 3 [wherein Z₃, R, R¹, R², R³ and ‘n’ are as defined with respect to a compound of formula (II)].

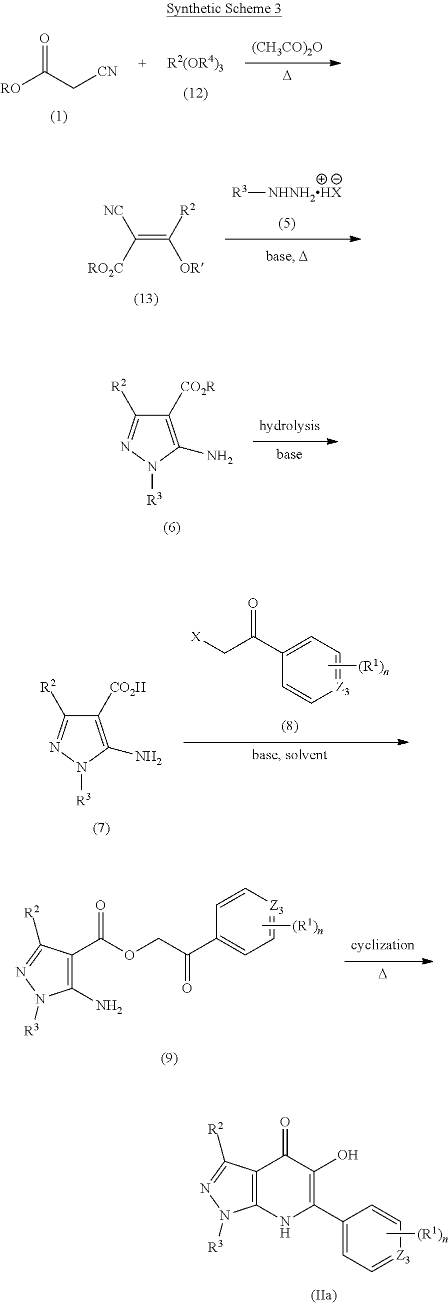

The reaction of alkyl cyanoacetate of formula (1) with trialky ortho derivative of the formula (12) [wherein R′ is C₁₋₄alkyl] using suitable reagent gives alkyl 2-cyano-3-alkoxyacrylate of the formula (13). In an embodiment the reaction carried out in suitable reagent. In an embodiment the suitable reagent is acetic anhydride. The intermolecular cyclization of compound of the formula (13) with alkyl or aryl hydrazine or its salts such as sulfate or hydrochloride of formula (5) in the presence of suitable base affords amino pyrazole ester of the formula (6). In an embodiment the reaction may be carried out in the presence of suitable solvent. In an embodiment the suitable solvent is ethanol. In an embodiment the suitably substituted hydrazine is methyl hydrazine. The ester hydrolysis of amino pyrazole ester of the formula (6) using a suitable base gives corresponding amino pyrazole carboxylic acid of formula (7). In an embodiment the reaction may be carried out in the presence of a suitable base. In an embodiment the suitable base is sodium hydroxide or potassium hydroxide. The coupling reaction of amino pyrazole carboxylic acid of the formula (7) with appropriately substituted phenacyl halide of the formula (8) using suitable base such affords compound of the formula (9). In an embodiment the reaction may be carried out in the presence of a suitable base. In an embodiment the suitable base may be potassium fluoride. In an embodiment the reaction may be carried out in the presence of suitable solvent. In an embodiment the suitable solvent may N,N′-dimethyl formamide. The intramolecular cyclization of compound of the formula (9) using a suitable dehydrating agent gives compound of general formula (IIa). In an embodiment the suitable dehydrating agent may be selected from polyphosphoric acid, phosphorous pentoxide, zinc chloride and sulfuric acid.

An alternative approach for synthesis pyrazolo[3,4-b]pyridinone compound of general formula (IIa) is depicted in synthetic scheme 4 [wherein Z₃, R¹, R², R³ and ‘n’ are as defined with respect to a compound of formula (II)].

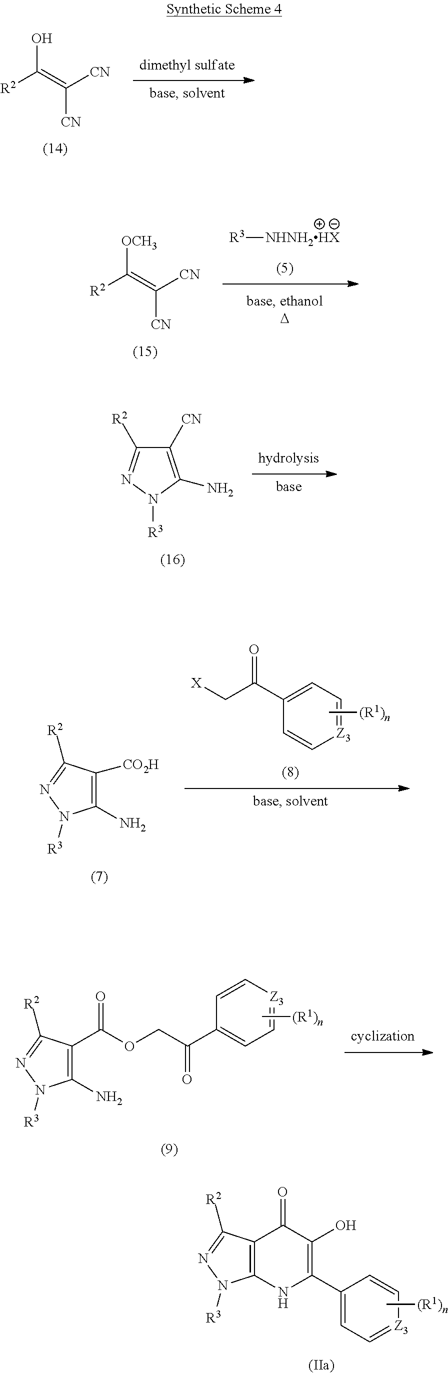

The starting material (14) can be prepared by a known method from the reaction of malononitrile with appropriately substituted acid chloride using suitable base in suitable solvent. The methylation of hydroxy dicyano (14) using dimethyl sulfate or methyl iodide in the presence of suitable base gives the corresponding methoxy dicyano derivative compound of formula (15). In an embodiment the reaction may be carried out in the presence of a suitable base. In an embodiment the suitable base may be sodium hydroxide. The intermolecular cyclization of compound of formula (15) with suitably substituted hydrazine salt of formula (5) in the presence of suitable base and suitable solvent affords 5-amino-4-cyanopyrazole compound of formula (16). In an embodiment the reaction may be carried out in the presence of suitable solvent. In an embodiment the suitable solvent is ethanol. In an embodiment the suitable base for the reaction may be selected from N,N-diisopropylethylamine or triethylamine. In an embodiment the suitably substituted hydrazine is methyl hydrazine. The aqueous hydrolysis of compound of the formula (16) using suitable base affords the pyrazole carboxylic acid of the formula (7). In an embodiment the reaction may be carried out in the presence of a suitable base. In an embodiment the suitable base is sodium hydroxide or potassium hydroxide. The coupling reaction of pyrazole carboxylic acid (7) with appropriately substituted halide compound of formula (8) using suitable base in suitable solvent affords compound of formula (9). In an embodiment the reaction may be carried out in the presence of a suitable base. In an embodiment the suitable base may be potassium fluoride. In an embodiment the reaction may be carried out in the presence of suitable solvent. In an embodiment the suitable solvent may N,N′-dimethyl formamide. The intramolecular cyclization of compound of formula (9) in the presence of suitable dehydrating agent gives compound of general formula (IIa). In an embodiment the suitable dehydrating agent may be selected from polyphosphoric acid, phosphorous pentoxide, zinc chloride and sulfuric acid.

Another approach for synthesis pyrazolo[3,4-b]pyridinone compound of general formula (IIa) is depicted in synthetic scheme 5 [wherein Z₃, R¹, R², R³ and ‘n’ are as defined with respect to a compound of formula (II)].

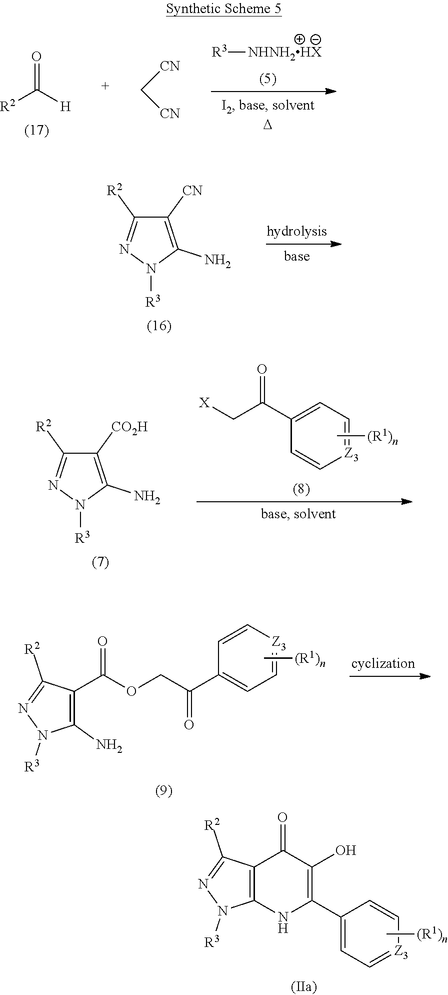

The reaction of appropriately substituted aldehyde of the formula (17) with malononitrile using suitable base followed by the intermolecular cyclization using suitably substituted hydrazine salt of formula (5) in the presence of iodine affords 5-amino-4-cyanopyrazole (16). In an embodiment the reaction may be carried out in the presence of a suitable base. In an embodiment the suitable base is sodium hydroxide or potassium hydroxide. In an embodiment the suitably substituted hydrazine is methyl hydrazine. The aqueous hydrolysis of 5 amino pyrazole nitrile (16) using basic or acidic conditions affords the pyrazole carboxylic acid of formula (7). The coupling reaction of pyrazole carboxylic acid (7) with appropriately substituted halide compound of formula (8) using suitable base affords compound of formula (9). In an embodiment the reaction may be carried out in the presence of a suitable base. In an embodiment the suitable base may be potassium fluoride. In an embodiment the reaction may be carried out in the presence of suitable solvent. In an embodiment the suitable solvent may N,N′-dimethyl formamide. The intramolecular cyclization of compound of formula (9) in the presence of suitable dehydrating agent gives compound of general formula (IIa). In an embodiment the suitable dehydrating agent may be selected from polyphosphoric acid, phosphorous pentoxide, zinc chloride and sulfuric acid.

An approach for synthesis of substituted 5-hydroxy-6-arylpyrano[2,3-c]pyrazol-4(1H)-one of general formula (Ia) [wherein R¹, R², R³ and ‘n’ are as defined with respect to a compound of formula (I)] is depicted in synthetic scheme 6.

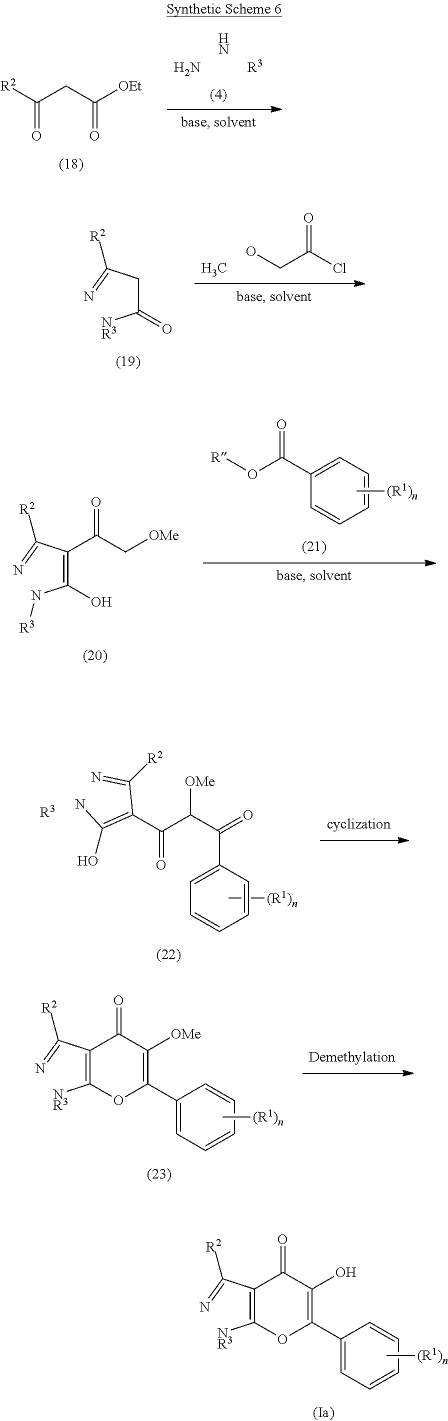

The reaction of β-keto ester derivative of formula (18) with an appropriately substituted hydrazine compound of formula (4) in the presence of a suitable base affords pyrazolone derivative of formula (19). In an embodiment the reaction may be carried out in the presence of a suitable base. In an embodiment the suitable base is sodium hydroxide or potassium hydroxide. In an embodiment, the reaction may be carried out in the presence of the suitably substituted hydrazine. In an embodiment the suitably substituted hydrazine is methyl hydrazine. The reaction of pyrazolone derivative of formula (19) with methoxyacetyl chloride in the presence of a suitable base gives pyrazolo-2-methoxyethanone deravative (20). In an embodiment the reaction may be carried out in the presence of a suitable base. In an embodiment the suitable base is calcium hydroxide. The Claisen condensation of pyrazolo-2-methoxyethanone (20) with aryl carboxylic acid ester of formula (21) in the presence of suitable base affords 5-hydroxy-pyrazolo-2-methoxy-3-arylpropane-1,3-dione of formula (22). In an embodiment the reaction may be carried out in the presence of a suitable base. In an embodiment the suitable base is sodium hydride. The intramolecular cyclization of (22) using suitable reagents gives the 5-methoxy-6-arylpyrano [2,3-c]pyrazol-4(1H)-one derivative (23). In an embodiment the reaction may be carried out in the presence of suitable reagents. In an embodiment the suitable reagents may be a mixture of sulfuric acid and acetic acid. The demethylation of compound of formula (23) using suitable reagent furnishes the 5-hydroxy-6-arylpyrano[2,3-c]pyrazole of general formula (Ia). In an embodiment the reaction may be carried out in the presence of suitable reagents. In an embodiment the suitable reagents may be boron tribromide or aqueous hydrobromic acid.

A general approach for synthesis of substituted pyrazolo[3,4-b]pyridinone compound of general formula (IIb) [wherein Z₃, R¹, R³ and ‘n’ are as defined with respect to a compound of formula (II) and wherein R″ is H, F, Cl, C₁₋₈alkyl, etc] is depicted in synthetic scheme 7.

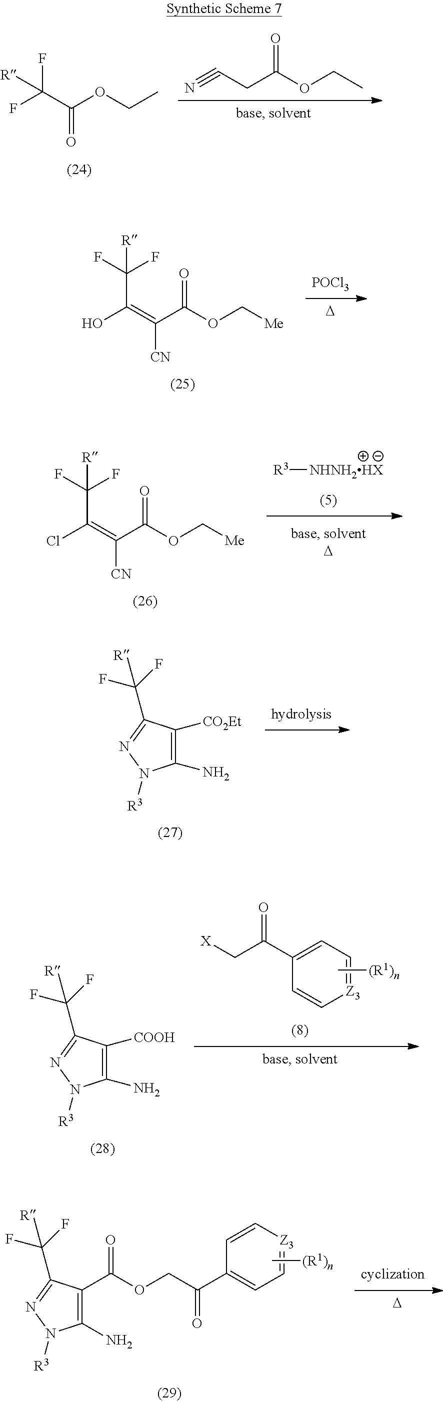

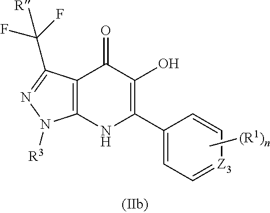

The hydroxy cyano intermediate of the formula (25) can be readily prepared by reaction of ethyl cyanoacetate with appropriately substituted ester of the formula (24) in the presence of suitable base. In an embodiment the reaction may be carried out in the presence of a suitable base. In an embodiment the suitable base may be sodium hydroxide or triethylamine. The reaction of (25) with phosphorus oxychloride gives the corresponding chloride compound of the formula (26).The intermolecular cyclization of compound of the formula (26) with alkyl or aryl hydrazine or its salts such as sulfate or hydrochloride of the formula (5) in the presence of suitable base affords amino pyrazole ester of the formula (27). In an embodiment the reaction may be carried out in the presence of suitable solvent. In an embodiment the suitable solvent is ethanol. In an embodiment the reaction may be carried out in the presence of a suitable base. In an embodiment the suitable base for the reaction may be selected from N,N-diisopropylethylamine or triethylamine. In an embodiment the reaction may be carried out in the presence of suitably substituted hydrazine. In an embodiment the suitably substituted hydrazine is methyl hydrazine. The ester hydrolysis of amino pyrazole ester (27) using a suitable base gives amino pyrazole caboxylic acid derivative of the formula (28). In an embodiment the reaction may be carried out in the presence of a suitable base. In an embodiment the suitable base is sodium hydroxide or potassium hydroxide. The coupling of amino pyrazole caboxylic acid derivative of the formula (28) with suitably substituted phenacyl halide of the formula (8) using suitable base affords compound of the formula (29). In an embodiment the reaction may be carried out in the presence of a suitable base. In an embodiment the suitable base is potassium fluoride. In an embodiment the reaction may be carried out in the presence of a suitable solvent. In an embodiment the suitable solvent is N,N′-dimethyl formamide. The intramolecular cyclization of compound of the formula (29) using a suitable dehydrating agent gives compound of general formula (IIb). In an embodiment the suitable dehydrating agent may be selected from polyphosphoric acid, phosphorous pentoxide, zinc chloride or sulfuric acid.

Another approach for synthesis pyrazolo[3,4-b]pyridinone compound of general formula (IIc) [wherein Z₃, R¹, R³ and ‘n’ are as defined with respect to a compound of formula (II)] is depicted in synthetic scheme 8.

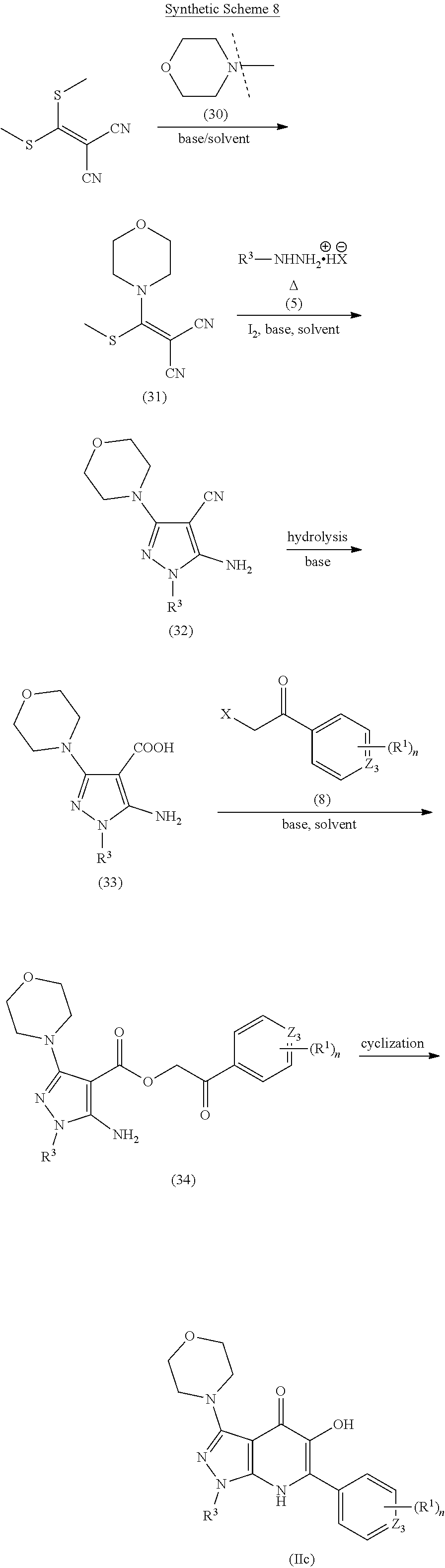

The 2-[Bis(methylthio)methylene]malononitrile can be prepared readily by the recation malononitrile with carbon disulphide and methyl iodide using potassium fluoride as base in dry DMF. The displacement recation of 2-[bis(methylthio)methylene]malononitrile with amine of the formula (30) with using suitable base and solvent affords bisnitrile compound of formula (31). The intermolecular cyclization of compound of formula (31) with suitably substituted hydrazine salt of formula (5) in the presence of suitable base and suitable solvent affords 5-amino-4-cyanopyrazole compound of formula (32). In an embodiment the reaction may be carried out in the presence of a suitable solvent. In an embodiment the suitable solvent is ethanol. In an embodiment the suitable base for the reaction may be selected from N,N-diisopropylethylamine or triethylamine. In an embodiment the suitably substituted hydrazine is methyl hydrazine. The hydrolysis of the cyano group of compound of formula (31) in the presence sitable base in a mixture of suitable solvent affords the carboxylic acid compound of formula (33). In an embodiment the suitable base for the reaction may be sodium hydroxide or potassium hydroxide. In an embodiment the reaction may be carried out in the presence of a mixture of suitable solvent. In an embodiment the mixture of suitable solvent is water and ethanol or water and methanol. In an embodiment a mixture of suitable solvent is in the appropriate proportion. In an embodiment the appropriate proportion is 1:3. The coupling reaction of pyrazole carboxylic acid (33) with appropriately substituted halide compound of formula (8) using suitable base affords compound of formula (34). In an embodiment the reaction may be carried out in the presence of a suitable base. In an embodiment the suitable base may be potassium fluoride. In an embodiment the reaction may be carried out in the presence of a suitable solvent. In an embodiment the suitable solvent may N,N′-dimethyl formamide. The intramolecular cyclization of compound of formula (34) in the presence of suitable dehydrating agent gives compound of general formula (IIc). In an embodiment the suitable dehydrating agent may be selected from polyphosphoric acid, phosphorous pentoxide, zinc chloride and sulfuric acid.

Another approach for synthesis of pyrazolo[3,4-b]pyridinone compound of general formula (lb) is depicted in synthetic scheme 9 [wherein Z₃, R, R¹, R², R³ and ‘n’ are as defined with respect to a compound of formula (I)].

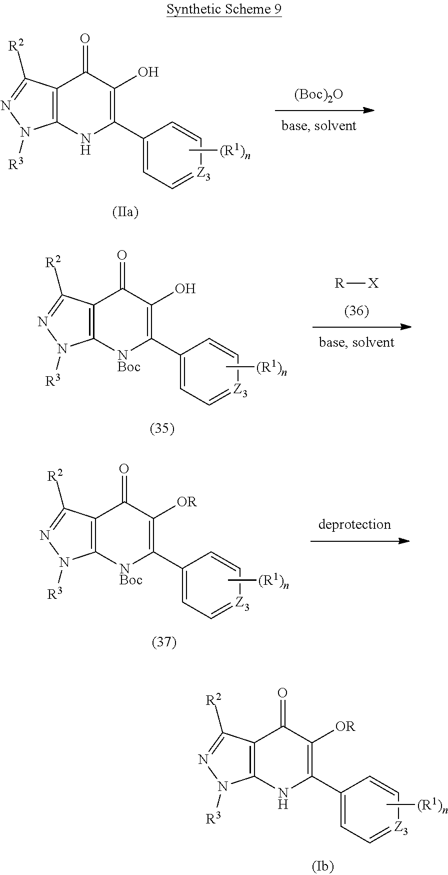

The amine group in compound of formula (11a) on reaction with with di-tert-butyl dicarbonate (BOC anhydride) in the presence of suitable base in a suitable solvent to gives the protected amine compound of formula (35). In an embodiment the reaction may be carried out in the presence of a suitable base. In an embodiment the suitable base is DMAP. In an embodiment the reaction may be carried out in the presence of a suitable solvent. In an embodiment the suitable solvent is THF. The 0-alkylation of compound (35) using suitable alkyl halide of the formula (36) [wherein R is C₁₋₈alkyl and X is Cl, F or I] in the presence of suitable base in a suitable solvent gives compound of formula (37). In an embodiment the reaction may be carried out in the presence of a suitable base. In an embodiment the suitable base is potassium carbonate. In an embodiment the reaction may be carried out in the presence of a suitable solvent. In an embodiment the suitable solvent is N,N′-dimethyl formamide. The deprotection of compound of formula (37) using trifluoroacetic acid in suitable solvent under acidic condition gives the compound of formula (Ib). In an embodiment the reaction may be carried out in the presence of suitable solvent. In an embodiment the suitable solvent is dichloromethane.

A general approach for synthesis of substituted pyrazolo[3,4-b]pyridinone compound of general formula (Ic) [wherein Z₃, R¹, R², R³ and ‘n’ are as defined with respect to a compound of formula (I)] is depicted in synthetic scheme 10.

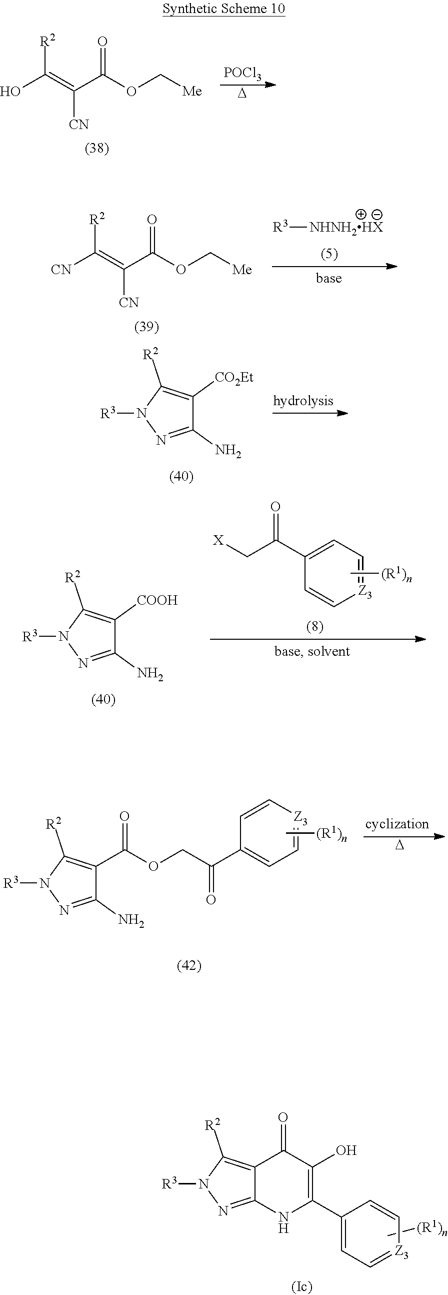

The starting material (38) can be prepared by the reaction of ethyl cyanoacetate with appropriately substituted acid chloride using suitable base in suitable solvent. In an embodiment the reaction may be carried out in the presence of a suitable solvent. In an embodiment the suitable solvent may be N,N′-dimethyl formamide or THF. The reaction of hydroxyl cyano ester (38) with phosphorus oxychloride gives the corresponding chloride compound of the formula (39). The intermolecular cyclization of compound of the formula (39) with alkyl or aryl hydrazine or its salts such as sulfate or hydrochloride of the formula (5) in the presence of suitable base affords amino pyrazole ester of the formula (40). In an embodiment the reaction may be carried out in the presence of a suitable solvent. In an embodiment the suitable solvent is ethanol. In an embodiment the reaction may be carried out in the presence of a suitable base. In an embodiment the suitable base may be triethylamine or N,N-diisopropyl ethyl amine. In an embodiment the reaction may be carried out in the presence of suitably substituted hydrazine. In an embodiment the suitably substituted hydrazine is methyl hydrazine. The ester hydrolysis of amino pyrazole ester (40) using a suitable base in a mixture of suitable solvent gives amino pyrazole caboxylic acid derivative of the formula (41). In an embodiment the reaction may be carried out in the presence of a suitable base. In an embodiment the suitable base may be potassium hydroxide or sodium hydroxide. In an embodiment the reaction may be carried out in the presence of a mixture of suitable solvents. In an embodiment the mixture of suitable solvent are water and ethanol or water and methanol. In an embodiment a mixture of suitable solvent is in the appropriate proportion. In an embodiment the appropriate proportion is 1:3. The coupling of amino pyrazole caboxylic acid derivative of the formula (41) with appropriately substituted phenacyl halide of the formula (8) using suitable base affords compound of the formula (42). In an embodiment the reaction may be carried out in the presence of a suitable base. In an embodiment the suitable base is potassium fluoride. In an embodiment the reaction may be carried out in the presence of suitable solvent. In an embodiment the suitable solvent is N,N′-dimethyl formamide. The intramolecular cyclization of compound of the formula (42) using a suitable dehydrating agent gives compound of general formula (Ic). In an embodiment the suitable dehydrating agent may be selected from polyphosphoric acid, phosphorous pentoxide, zinc chloride and sulfuric acid.

An alternative approach for synthesis of substituted pyrazolo[3,4-b]pyridinone compound of general formula (Ic) [wherein Z₃, R¹, R², R³ and ‘n’ are as defined with respect to a compound of formula (I)] is depicted in synthetic scheme 11.

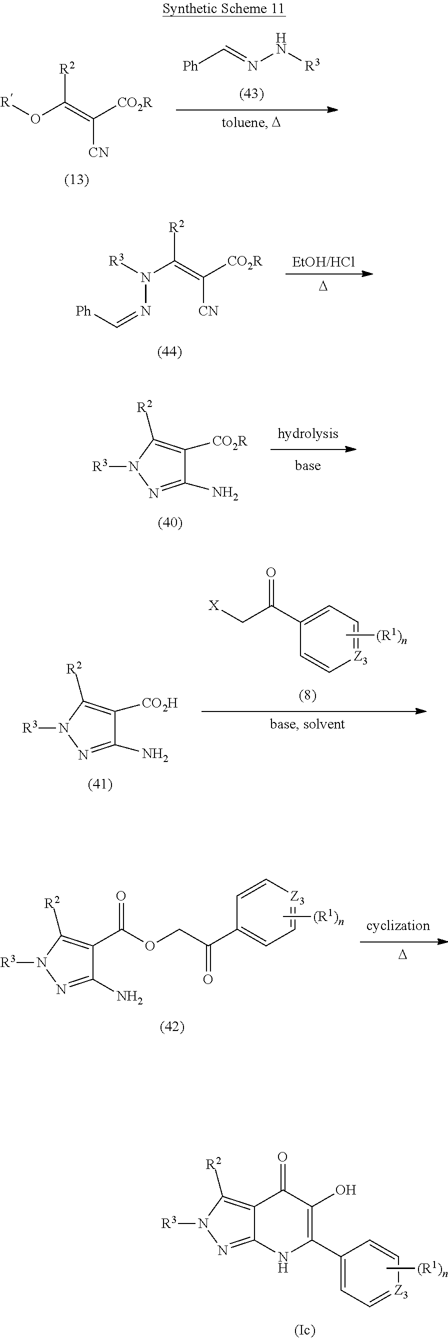

The reaction of ethyl 2-cyano-3-alkoxyacrylate of formula (13) [wherein R′ is C₁₋₈alkyl] with appropriately substituted hydrazone compound of formula (43) affords compound of the formula (44). The cyclization of (44) under acidic condition gives amino pyrazole ester (40), which on ester hydrolysis as described in scheme 10 gives amino pyrazole carboxylic acid derivative (41). The coupling reaction of pyrazole carboxylic acid (41) with an appropriately substituted phenacyl halide of the formula (8) using suitable affords compound of formula (42). In an embodiment the reaction may be carried out in the presence of a suitable base. In an embodiment the suitable base may be potassium fluoride. In an embodiment the reaction may be carried out in the presence of a suitable solvent. In an embodiment the suitable solvent may N,N′-dimethyl formamide. The intramolecular cyclization of the compound of formula (42) using a suitable dehydrating agent gives compound of general formula (Ic). In an embodiment the suitable dehydrating agent may be selected from polyphosphoric acid, phosphorous pentoxide, zinc chloride and sulfuric acid.

A general approach for the synthesis of thieno[2,3-b]pyridinone of general formula (Id) [wherein Z₃, R¹, R², R⁴ and ‘n’ are as defined with respect to a compound of formula (I)] is depicted in synthetic scheme 12.

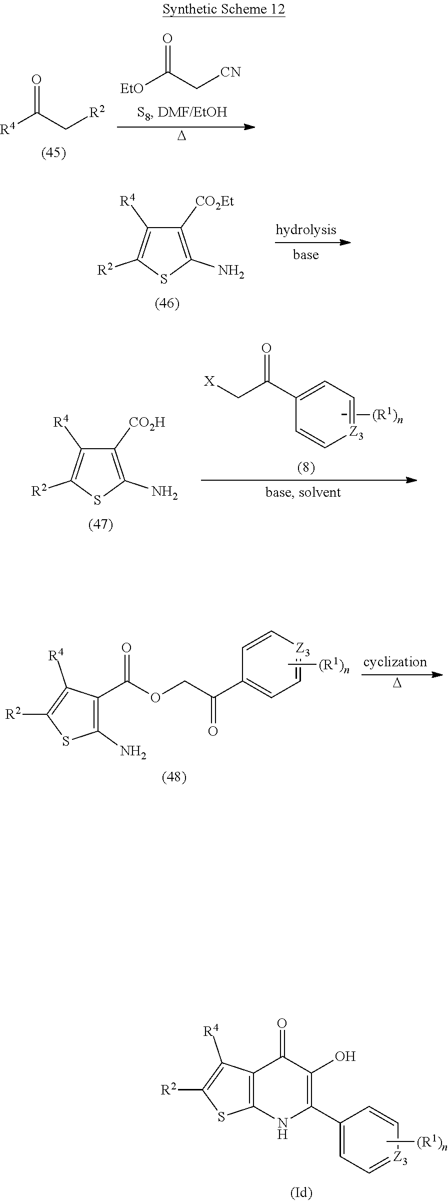

The 2-amino-thiophene ester of formula (46) was prepared using compound of formula (45) with ethyl cyanoacetate using sulfur powder. The ester hydrolysis of 2-amino thiophene ester compound of formula (46) using a suitable base affords amino thiophene carboxylic acid compound of formula (47). In an embodiment the reaction may be carried out in the presence of a suitable base. In an embodiment the suitable base may be lithium hydroxide, potassium hydroxide or sodium hydroxide. In an embodiment the reaction may be carried out in the presence of solvent such as ethanol, isopropanol, etc. The coupling reaction of thiophene carboxylic acid (47) with an appropriately substituted phenacyl halide compound of formula (8) using suitable base affords compound of formula (48). In an embodiment the reaction may be carried out in the presence of a suitable base. In an embodiment the suitable base may be potassium fluoride. In an embodiment the reaction may be carried out in the presence of a suitable solvent. In an embodiment the suitable solvent may N,N′-dimethyl formamide. The intramolecular cyclization of compound of formula (48) using a suitable dehydrating agent furnishes thieno[2,3-b]pyridinone compound of general formula (Id). In an embodiment the suitable dehydrating agent may be selected from polyphosphoric acid, phosphorous pentoxide, zinc chloride and sulfuric acid.

A general approach for synthesis of substituted pyrazolo[3,4-b]pyridinone compound of general formula (Ie) [wherein Z₃, R¹, R², R³ and ‘n’ are as defined with respect to a compound of formula (I)] is depicted in synthetic scheme 13.

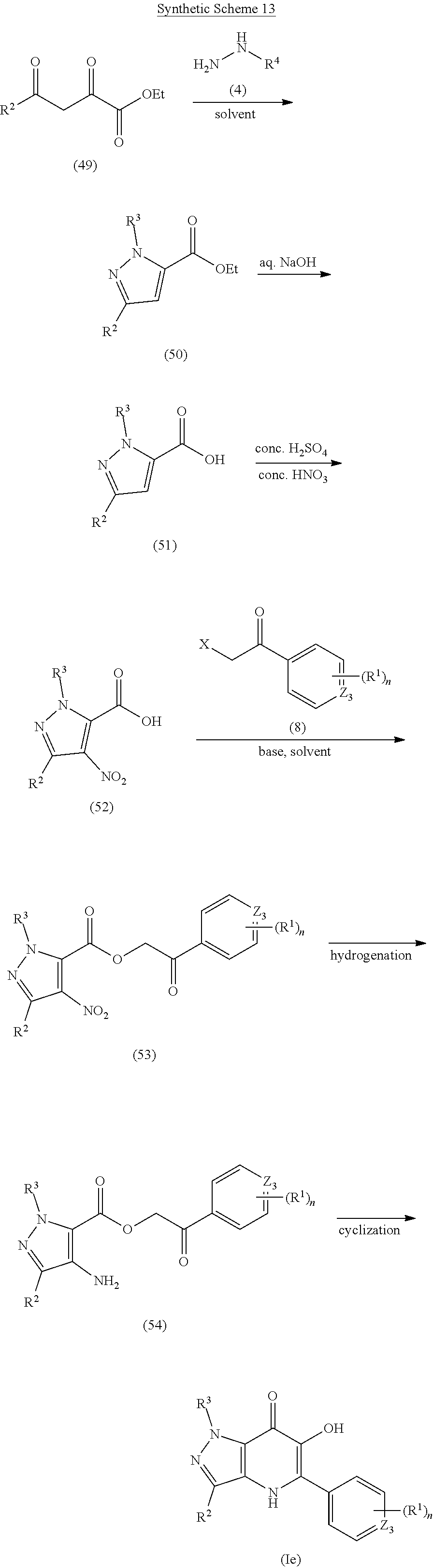

The cyclization of diketoester of the formula (49) with appropriately substituted hydrazine compound of formula (4) affords pyrazole-5-carboxylate compound of formula (50). The ester hydrolysis of pyrazole-5-carboxylate (50) using suitable base affords corresponding pyrazole carboxylic acid of formula (51). In an embodiment the reaction may be carried out in the presence of a suitable base. In an embodiment the suitable base is sodium hydroxide or potassium hydroxide. The nitration of compound of formula (51) using concentrated sulfuric acid and fuming nitric acid results in formation of the corresponding 4-nitro-1H-pyrazole-5-carboxylic acid derivative (52). The coupling reaction of nitro pyrazole acid (52) with appropriately substituted phenacyl halide of the formula (8) using suitable base affords nitro pyrazole derivative of the formula (53). In an embodiment the reaction may be carried out in the presence of a suitable base. In an embodiment the suitable base may be potassium fluoride. The reduction of nitro pyrazole ester derivative (53) on catalytic hydrogenation in an appropriate solvent gives amino pyrazole ester derivatives of formula (54). In an embodiment the reaction may be carried out in the presence of solvent such as ethanol, methanol, ethyl acetate etc. The intramolecular cyclization of the amino pyrazole ester (54) using a suitable dehydrating agent gives pyrazolo[4,3-b]pyridinone of general formula (Ie). In an embodiment the suitable dehydrating agent may be selected from polyphosphoric acid, phosphorous pentoxide, zinc chloride and sulfuric acid.

A general approach for the synthesis of thiazolo[5,4-b]pyridinone of general formula (If) [wherein Z₃, R¹, R² and ‘n’ are as defined with respect to a compound of formula (I)] is depicted in synthetic scheme 14.

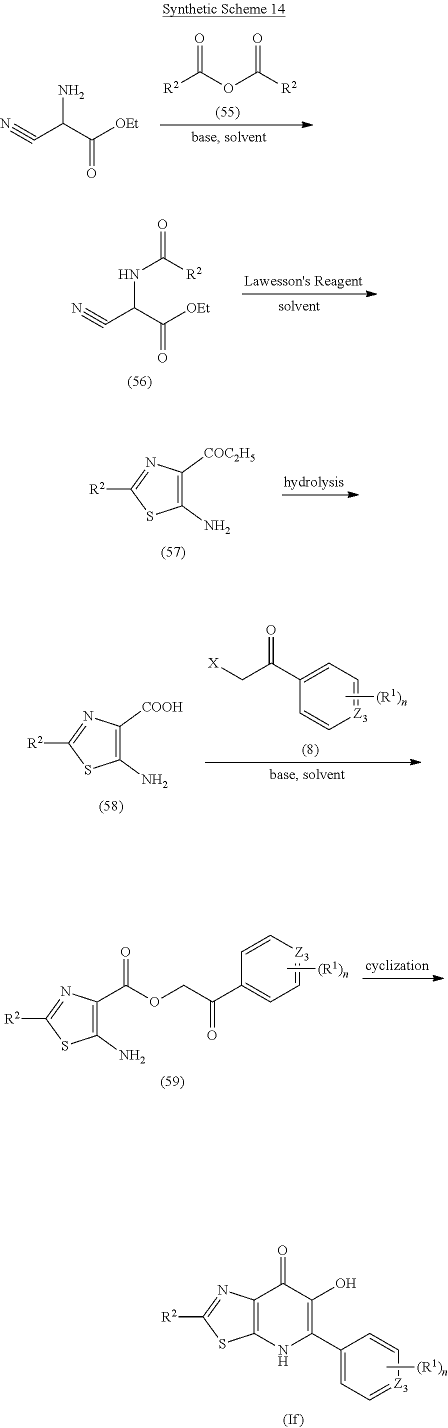

The acylation of ethyl 2-amino-2-cyanoacetate with suitable anhydride of the formula (55) in the presence of base affords acyl derivative of 2-amino-2-cyanoacetate (56). In an embodiment the reaction may be carried out in the presence of a suitable base. In an embodiment the suitable base may be dry pyridine. The cyclization of acyl amino derivative of the formula (56) using Lawes son' s reagent gives 5-amino-2-alkylthiazole-4-carboxylate (57). In an embodiment the reaction may be carried out in the presence of solvent. In an embodiment the suitable solvent may be selected from pyridine, toluene, THF, etc. The ester hydrolysis of compound (57) by using a suitable base affords 5-amino-2-alkylthiazole-4-carboxylic acid (58). In an embodiment the reaction may be carried out in the presence of a suitable base. In an embodiment the suitable base may be lithium hydroxide, potassium hydroxide or sodium hydroxide. The coupling reaction of amino thiazole carboxylic acid (58) with appropriately substituted phenacyl halide compound of formula (8) using suitable base affords compound of formula (59). In an embodiment the reaction may be carried out in the presence of a suitable base. In an embodiment the suitable base may be potassium fluoride. The intramolecular cyclization of compound of formula (59) using a suitable dehydrating agent furnishes thiazolo[5,4-b]pyridin-7(4H)-one of the general formula (If). In an embodiment the suitable dehydrating agent may be selected from polyphosphoric acid, phosphorous pentoxide, zinc chloride or sulfuric acid.

An approach for synthesis of substituted 6-hydroxy-3-methyl-5-aryl pyrano [3,2-c]pyrazol-7-one compound of general formula (Ig) [wherein R¹, R³ and ‘n’ are as defined with respect to a compound of formula (I)] is depicted in synthetic scheme 15.

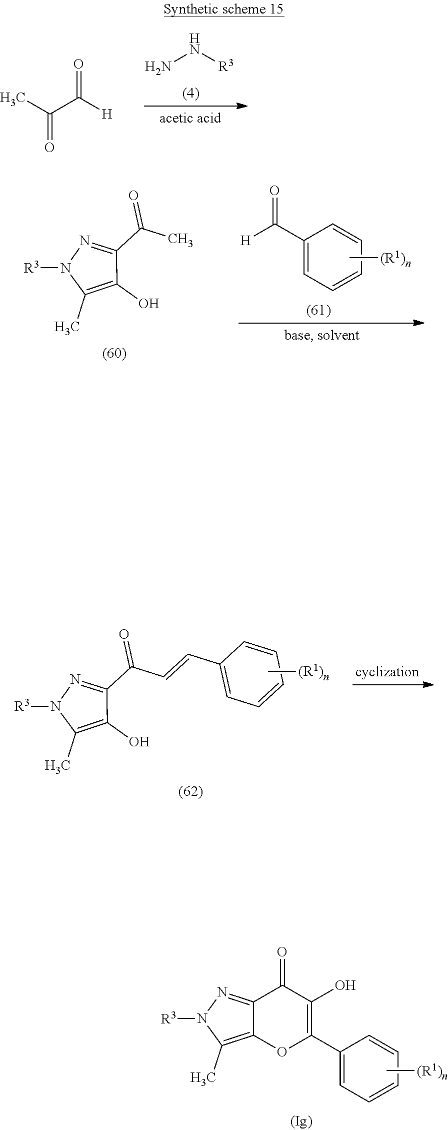

The 4-hydroxyl-3-acetyl pyrazole derivative of formula (60) can be prepared by the reaction of methyl glyoxal with appropriately substituted hydrazine compound of formula (4) in the presence of acetic acid. The reaction of 4-hydroxyl-3-acetyl pyrazole (60) with substituted aromatic aldehyde (61) in the presence of a suitable base affords substituted pyrazolo chalcone derivative of formula (62). In an embodiment the reaction may be carried out in the presence of a suitable base. In an embodiment the suitable base may be potassium hydroxide or sodium hydroxide. In an embodiment the reaction may be carried out in the presence of solvent such as ethanol, methanol, THF, isopropanol, etc. The intramolecular cyclization of compound of formula (62) using hydrogen peroxide and suitable base furnishes 6-hydroxy-3-methyl-5-arylpyrano[3,2-c]pyrazol-7-one of general formula (Ig). In an embodiment the reaction may be carried out in the presence of a suitable base. In an embodiment the suitable base may be potassium hydroxide or sodium hydroxide.

A general approach for the synthesis of 5-hydroxy-oxazolo[5,4-b]pyridine-4-one of general formula (Ih) [wherein Z₃, R¹, R² and ‘n’ are as defined with respect to a compound of formula (I)] is depicted in synthetic scheme 16.

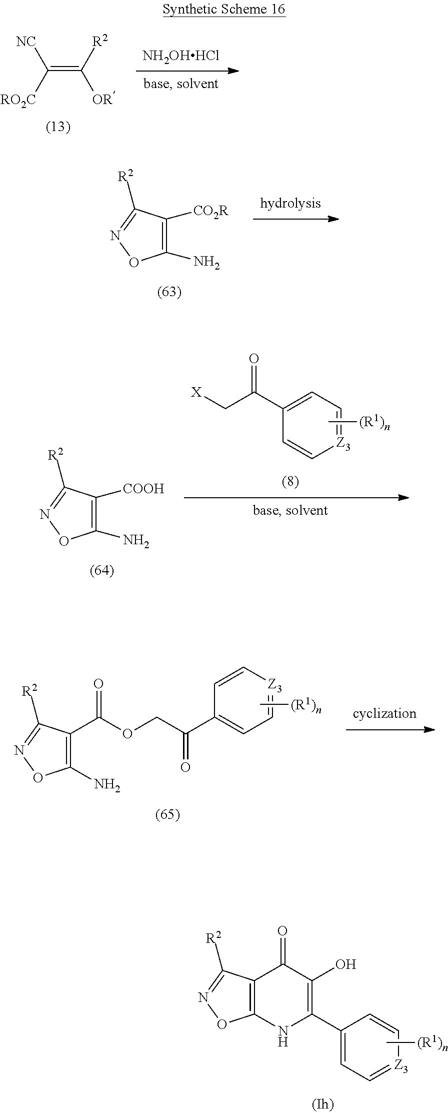

The ethyl 5-amino-3-alkylisoxazole-4-carboxylate of formula (63) can be prepared by the recation of 2-cyano-3-ethoxyalkyl-2-enoate derivative (13) with hydroxylamine hydrochloride using suitable base and solvent. In an embodiment the reaction may be carried out in the presence of a suitable base. In an embodiment the suitable base is potassium fluoride. In an embodiment the reaction may be carried out in the presence of a suitable solvent. In an embodiment the suitable solvent is N,N′-dimethyl formamide. The base mediated aqueous hydrolysis of compound of formula (63) gives corresponding amino isoxazole carboxylic acid compound of formula (64). The coupling reaction of amino isoxazole acid compound of formula (64) with appropriately substituted phenacyl halide compound of formula (8) using suitable base affords compound of formula (65). In an embodiment the reaction may be carried out in the presence of a suitable base. In an embodiment the suitable base is potassium fluoride. The intramolecular cyclization of compound of formula (65) using a suitable dehydrating agent furnishes 5-hydroxy-oxazolo[5,4-b]pyridine-4-one of general formula (Ih). In an embodiment the suitable dehydrating agent may be selected from polyphosphoric acid, phosphorous pentoxide, zinc chloride and sulfuric acid.

A general approach for synthesis imidazo[4,5-b]pyridin-7-one of general formula (Ii) is depicted in synthetic scheme 17 [wherein Z₃, R¹, R², R³ and ‘n’ are as defined with respect to a compound of formula (I)].

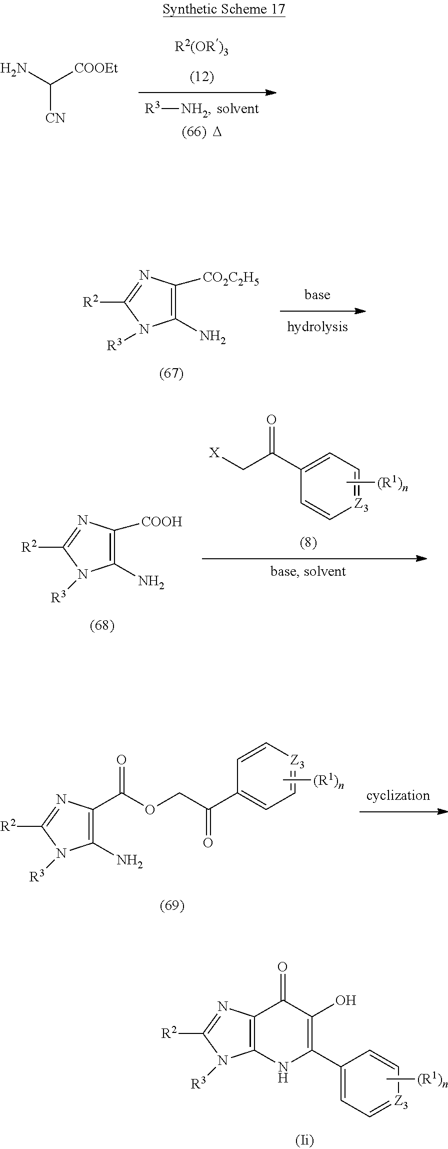

The 5-amino-imidazole-4-carboxylate of formula (67) was prepared by coupling reaction of ethyl 2-amino-2-cyanoacetate with amine of the formula (66) and trialkyl ortho derivative of the formula (12) under reflux condition using suitable solvent. In an embodiment the reaction may be carried out in the presence of a suitable solvent. In an embodiment the suitable solvent is acetonitrile. The ester hydrolysis of compound (67) using a suitable base affords the corresponding carboxylic acid compound of formula (68). In an embodiment the reaction may be carried out in the presence of a suitable base. In an embodiment the suitable base may be potassium hydroxide or sodium hydroxide. The coupling reaction of carboxylic acid compound of formula (68) with an appropriately substituted halide compound of formula (8) using suitable base affords the compound of formula (69). In an embodiment the reaction may be carried out in the presence of a suitable base. In an embodiment the suitable base is potassium fluoride. The intramolecular cyclization of the compound of formula (69) using a suitable dehydrating agent furnishes the imidazo[4,5-b]pyridin-7-one compound of general formula (Ii). In an embodiment the suitable dehydrating agent may be selected from polyphosphoric acid, phosphorous pentoxide, zinc chloride andsulfuric acid.

An approach for synthesis of thiazolo[4,5-b]pyridine-3-carboxamide compound of general formula (Ij) [wherein Z₃, R¹ and ‘n’ are as defined with respect to a compound of formula (I)] is depicted in synthetic scheme 18.

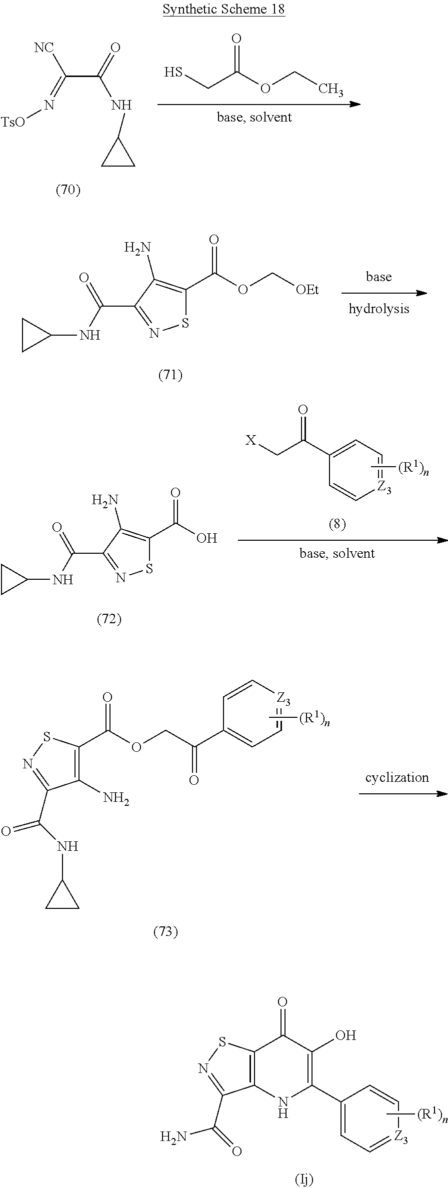

2-(Cyclopropylamino)-2-oxo-N-(tosyloxy)acetimidoyl cyanide (70) can be prepared from reaction of 2-cyano-N-cyclopropylacetamide with of NaNO2 in the precence of acetic acid and water followed by tosylation using p-toluenesulphonyl chloride in the presence of suitable base. The reaction of 2-(cyclopropylamino)-2-oxo-N-(tosyloxy)acetimidoyl cyanide (70) with ethyl 2-mercaptoacetate in the presence of suitable base affords ethoxymethyl 4-amino-3-(cyclopropylcarbamoyl)-1,2-thiazole-5-carboxylate (71). In an embodiment the reaction may be carried out in the presence of a suitable base. In an embodiment the suitable base may be piperidine or morpholine. In an embodiment the reaction may be carried out in the presence of a suitable solvent such as ethanol, methanol, or THF, etc. The ester hydrolysis of compound (71) using a suitable base affords the corresponding carboxylic acid compound of formula (72). In an embodiment the reaction may be carried out in the presence of a suitable base. In an embodiment the suitable base may be potassium hydroxide or sodium hydroxide. In an embodiment the reaction may be carried out in the presence of solvent such as ethanol , isopropanol, etc. The coupling reaction of carboxylic acid compound of formula (72) with an appropriately substituted halide compound of formula (8) using suitable base affords the compound of formula (73). In an embodiment the reaction may be carried out in the presence of a suitable base. In an embodiment the suitable base is potassium fluoride. The intramolecular cyclization of the compound of formula (73) using a suitable dehydrating agent affords the thiazolo[4,5-b]pyridine-3-carboxamide compound of general formula (Ij). In an embodiment the suitable dehydrating agent may be selected from polyphosphoric acid, phosphorous pentoxide, zinc chloride and sulfuric acid.

A general approach for the synthesis of pyrazolo[3,4-b]pyridinone of formula (IIe) [wherein Z₃, R¹, R ³ and ‘n’ are as defined with respect to a compound of formula (II)] is depicted in synthetic scheme 19.

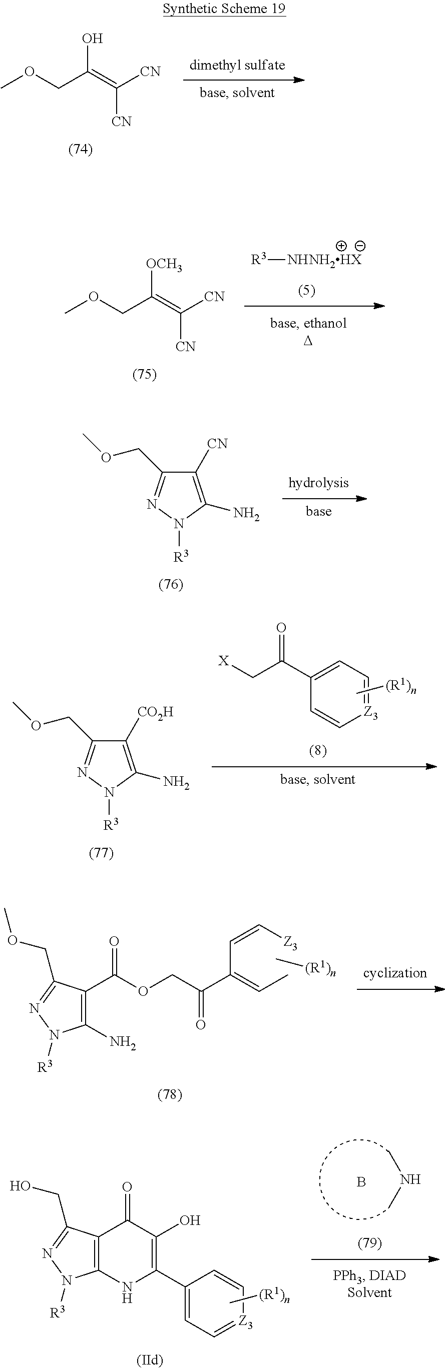

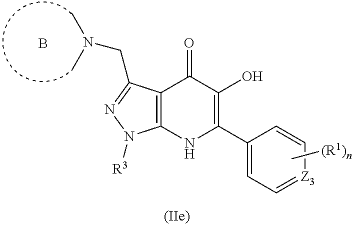

The starting material (74) can be prepared by a known method from the reaction of malononitrile with methoxy acetyl chloride using suitable base in suitable solvent. The methylation of hydroxy dicyano (74) using dimethyl sulfate or methyl iodide in the presence of suitable base gives 2-(1,2-dimethoxyethylidene)malononitrile (75). In an embodiment the suitable base may be sodium hydride or potassium tertiary butoxide or sodium tertiary butoxide. The cyclization of (75) with suitably substituted hydrazine or its salt of formula (5) in the presence of suitable base and suitable solvent affords 5-amino-4-cyanopyrazole compound of formula (76). In an embodiment the reaction may be carried out in the presence of suitable solvent. In an embodiment the suitable solvent is ethanol. In an embodiment the suitable base for the reaction may be selected from N,N-diisopropylethylamine or triethylamine. In an embodiment the suitably substituted hydrazine is methyl hydrazine. The aqueous hydrolysis of compound of the formula (76) using procedure describe in Scheme 4 gives pyrazole carboxylic acid of the formula (77). The coupling reaction of pyrazole carboxylic acid (77) with appropriately substituted halide compound of formula (8) using suitable base in suitable solvent affords compound of formula (78). The cyclization of compound of formula (78) in the presence of suitable dehydrating agent gives compound of general formula (IId). In an embodiment the suitable dehydrating agent may be selected from polyphosphoric acid, phosphorous pentoxide, zinc chloride and sulfuric acid. The Mitsunobu reaction of compound of formula (IId) with compound of formula (79) [wherein ring B is 3- to 15-membered heterocyclylC₁₋₈alkyl optionally substituted with one or more substituents independently selected from halogen, C₁₋₈alkyl, haloC₁₋₈alkyl, —S(O)₂CH₃, C₃₋₁₂cycloalkyl and 3- to 15-membered heterocyclyl] gives pyrazolo[3,4-b]pyridinone of formula (IIe). In an embodiment the suitable reagent for the reaction may be selected from triphenylphosphine and diethyl azodicarboxylate (DEAD) or diisopropyl azodicarboxylate (DIAD). In an embodiment the suitable solvent for the reaction may be selected such as THF, DMF or dioxane etc.

A general approach for the synthesis of pyrazolo[3,4-b]pyridinone of general formula (II) [wherein Z₃, R, R¹, R², R³ and ‘n’ are as defined with respect to a compound of formula (II)] is depicted in synthetic scheme 20.

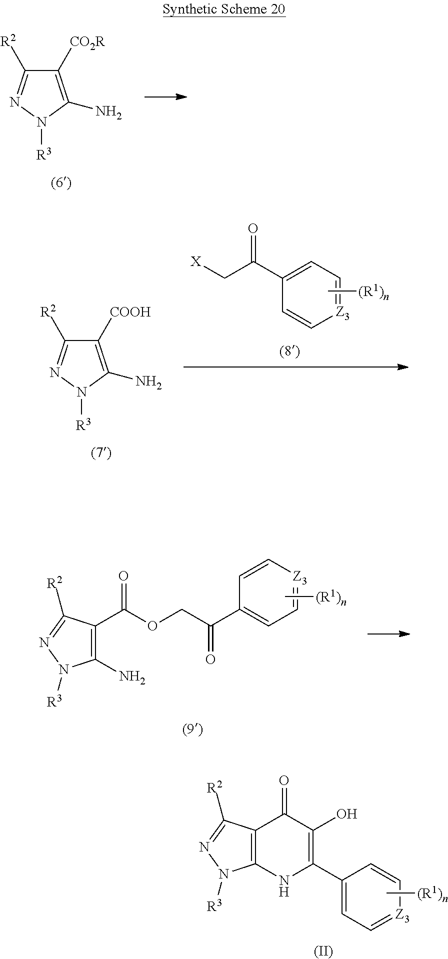

The process for the preparation of compound of formula (II) or a pharmaceutically acceptable salt thereof, the process comprising:

(i) hydrolysing the compound of formula (6′) to afford compound of formula (7′);

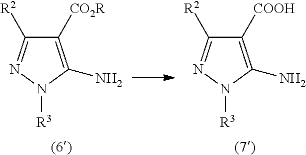

(ii) reacting the compound of formula (7′) with compound of formula (8′) to afford the compound of formula (9′)

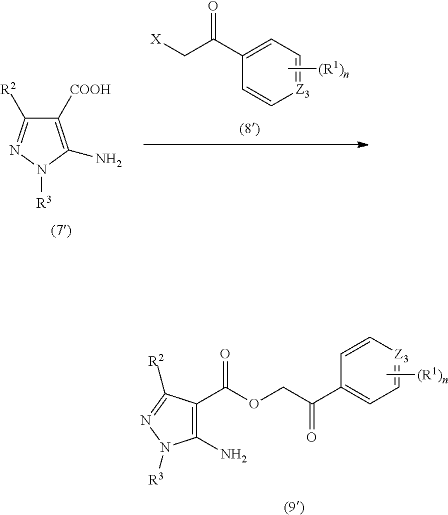

(iii) Converting the compound of formula (9′) to afford the compound of the general formula (IIa);

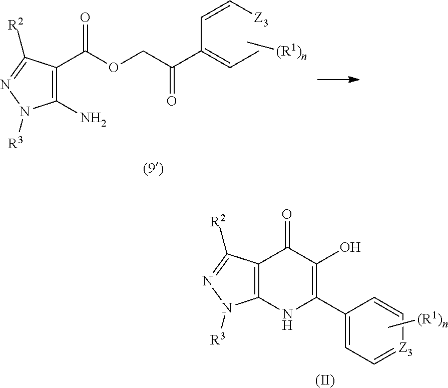

(iv) optionally converting the compound of the general formula (II) to a pharmaceutically acceptable salt thereof.

In an embodiment, the reaction of the compound of formula (6′) is carried out in presence of the suitable base.

In another embodiment, the suitable base is potassium hydroxide or sodium hydroxide. In yet another embodiment, the reaction of the compound of formula (6′) is carried out in presence of mixture of the suitable solvent.

In yet another embodiment, the mixture of the suitable solvent is water and ethanol or water and methanol.

In yet another embodiment, the mixture of the suitable solvent is in the appropriate proportion.

In yet another embodiment, the appropriate proportion is 1:3.

In yet another embodiment, the reaction of compound of formula (7′) is carried out in presence of the suitable base.

In yet another embodiment, the suitable base is potassium fluoride.

In yet another embodiment, the reaction of compound of formula (7′) is carried out in presence of the suitable solvent.

In yet another embodiment, the suitable solvent is N,N′-dimethyl formamide. In yet another embodiment, the reaction of compound of formula (9′) is carried out in presence of the suitable dehydrating agent.

In yet another embodiment, the suitable dehydrating agent is polyphosphoric acid, phosphorous pentoxide, zinc chloride or sulfuric acid.

### Experimental

Unless otherwise stated, work-up implies the following operations: distribution of the reaction mixture between the organic and aqueous phase, separation of layers, drying the organic layer over sodium sulfate, filtration and evaporation of the organic solvent. Purification, unless otherwise mentioned, implies purification by silica gel chromatographic techniques, generally using ethyl acetate/petroleum ether mixture of a suitable polarity as the mobile phase.

The following abbreviations are used in the text: DMSO-d₆: hexadeuterodimethyl sulfoxide; DMF: N,N-dimethylformamide, ¹H NMR: Proton Nuclear Magnetic Resonance; MS: Mass Spectrum; Ex.: Example; CDCl₃: Deuterated chloroform; CD₃COCD₃: Deuterated acetone; THF: Tetrahydrofuran; J: coupling constant in units of Hz; RT or rt: room temperature (22-26° C.); h: hour(s); min: minute(s); The starting materials used herein are commercially available or were prepared by methods known in the art to those of ordinary skill or by methods disclosed herein.

The intermediates described below were prepared using synthetic schemes 1 to 20 depicted above.

### Intermediates

### Intermediate 1

2-(2-Chlorophenyl)-2-oxoethyl 5-amino-1-methyl-1H-pyrazole-4-carboxylate

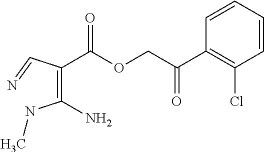 [SMILES: CN/C(N)=C(\C=N)C(=O)OCC(=O)c1ccccc1Cl]

### Step 1: Ethyl-2-cyano-3-(dimethylamino)prop-2-enoate

A mixture of ethyl cyanoacetate (10.0 g, 88.40 mmol) and N,N′-dimethylformamide dimethyl acetal (15.31 mL, 114.92 mmol) was refluxed in dry methanol (100 mL) for 3 h. The reaction mixture was cooled to room tempetrature and concentrated under reduced pressure. The residue obtained was diluted with water and extracted with ethyl acetate (2×250 mL) and the organic layer was dried over anhydrous sodium sulfate. The solution was concentrated under reduced pressure and the residue obtained was purified by flash silica gel column chromatography to afford 15.2 g of the product as a solid. ¹H NMR (300 MHz, CDCl₃): δ 1.30 (t, J=7.5 Hz, 3H), 3.21 (s, 3H), 3.38 (s, 3H), 4.22 (q, J=6.9 Hz, 2H), 7.69 (s, 1H).

### Step 2: Ethyl 5-amino-1-methyl-1H-pyrazole-4-carboxylate

A mixture of Step 1 intermediate (6.0 g, 35.670 mmol) and methyl hydrazine (1.9 mL, 35.670 mmol) was refluxed in dry ethanol (60 mL) overnight. The reaction mixture was cooled to room temperature and concentrated under reduced pressure and the residue thus obtained was diluted with water (150 mL). The aqueous layer was extracted with ethyl acetate (3×150 mL). The combined organic extracts were washed with water (150 ml) and dried over anhydrous sodium sulfate. The solution was concentrated under reduced pressure and the residue thus obtained was purified by flash silica gel column chromatography to afford 4.82 g of the titled product as a solid. ¹H NMR (300 MHz DMSO-d₆): δ 1.23 (t, J=7.5 Hz, 3H), 3.52 (s, 3H), 4.15 (q, J=6.6 Hz, 2H), 6.19 (br s, 2H), 7.41 (s, 1H).

### Step 3: 5-Amino-1-methyl-1H-pyrazole-4-carboxylic acid

To a stirred solution of step 2 intermediate (4.8 g, 28.37 mmol) in ethanol (28 mL), aqueous solution of potassium hydroxide (2.0 M, 28 mL, 42.555 mmol) was added and the reaction mixture was refluxed for overnight. The reaction mixture was cooled to RT, concentrated under reduced pressure. The residue was stirred in 1.0 N citric acid (80 mL). The solid precipitated was filtered and dried to yield 3.59 g of the titled product. ¹H NMR (300 MHz DMSO-d₆): δ 3.51 (s, 3H), 6.13 (br s, 2H), 7.38 (s, 1H), 11.74 (s, 1H).

### Step 4: 2-(2-Chlorophenyl)-2-oxoethyl 5-amino-1-methyl-1H-pyrazole-4-carboxylate

To a stirred solution of Step 3 intermediate (800 mg, 5.666 mmol) in dry DMF (8 ml), 2-bromo-1-(2-chlorophenyl)ethanone (1.32 g, 5.666 mmol) was added followed by potassium fluoride (500 mg, 8.499 mmol) at room temperature and the resultant reaction mixture was stirred overnight. The mixture was quenched with water (75 mL) and ethyl acetate (30 mL). The layers were separated and the aqueous layer was extracted with ethyl acetate (3×100 mL). The combined organic layers were washed with water (2×100 mL) and dried over anhydrous sodium sulfate. The solvent was distilled off under reduced pressure and the residue obtained was purified by flash silica gel column chromatography to afford 912 mg of the desired product as a solid. ¹H NMR (300 MHz, DMSO-d₆): δ 3.54 (s, 3H), 5.31 (s, 2H), 6.31 (s, 2H), 7.43-7.53 (m, 1H), 7.55 (d, J=4.8 Hz, 1H), 7.77 (d, J=7.5 Hz, 2H), 7.95 (s, 1H).

### Intermediate 2

2-(3-Chlorophenyl)-2-oxoethyl 5-amino-1-methyl-1H-pyrazole-4-carboxylate

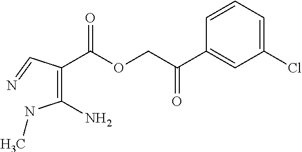 [SMILES: CN/C(N)=C(\C=N)C(=O)OCC(=O)c1cccc(Cl)c1]

The titled intermediate was prepared by the reaction of 5-amino-1-methyl-1H-pyrazole-4-carboxylic acid (700 mg, 4.93 mmol) with 2-bromo-1-(3-chlorophenyl)ethanone (1.38 g, 5.92 mmol) using potassium fluoride (430 mg, 7.40 mmol) in dry DMF (7.0 mL) as per the procedure described in Step 4 of Intermediate 1 to yield 680 mg of the product as a solid. ¹H NMR (300 MHz, CDCl₃): δ 3.64 (s, 3H), 5.08 (br s, 2H), 5.42 (s, 2H), 7.45 (t, J=7.8 Hz, 1H), 7.60 (d, J=8.7 Hz, 1H), 7.71 (s, 1H), 7.83 (d, J=7.8 Hz, 1H), 7.93 (s, 1H).

### Intermediate 3

2-(2,4-Dichlorophenyl)-2-oxoethyl 5-amino-1-methyl-1H-pyrazole-4-carboxylate

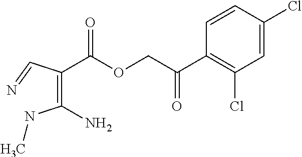 [SMILES: CN/C(N)=C(\C=N)C(=O)OCC(=O)c1ccc(Cl)cc1Cl]

The titled intermediate was prepared by the reaction of 5-amino-1-methyl-1H-pyrazole-4-carboxylic acid (800 mg, 5.66 mmol) with 2-bromo-1-(2,4-dichlorophenyl)ethanone (1.81 g, 6.77 mmol) using potassium fluoride (494 mg, 8.49 mmol) in dry DMF (8.0 mL) as per the procedure described in Step 4 of Intermediate 1 to yield 1.18 g of the product as a solid. ¹H NMR (300 MHz, CDCl₃): δ 3.54 (s, 3H), 5.30 (s, 2H), 6.32 (s, 2H), 7.47 (s, 1H), 7.61 (d, J=8.4 Hz, 1H), 7.81 (t, J=8.7 Hz, 1H), 7.95 (s, 1H), 7.95 (s, 1H).

### Intermediate 4

2-[4-Fluoro-3-(trifluoromethyl)phenyl]-2-oxoethyl 5-amino-1-methyl-1H-pyrazole-4-carboxylate

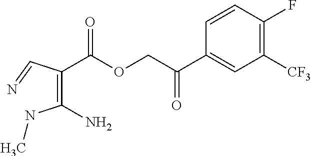 [SMILES: CN/C(N)=C(\C=N)C(=O)OCC(=O)c1ccc(F)c(C(F)(F)F)c1]

The titled intermediate was prepared by the reaction of 5-amino-1-methyl-1H-pyrazole-4-carboxylic acid (500 mg, 3.54 mmol) with 2-bromo-1-[4-fluoro-3-(trifluoromethyl)phenyl]ethanone (1.21 g, 4.24 mmol) using potassium fluoride (309 mg, 5.31 mmol) in dry DMF (5.0 mL) as per the procedure described in Step 4 of Intermediate 1 to yield 597 mg of the product as a solid. ¹H NMR (300 MHz, DMSO-d₆): δ 3.55 (s, 3H), 5.57 (s, 2H), 6.31 (s, 2H), 7.52 (s, 1H), 7.74 (t, J=9.6 Hz, 1H), 8.31 (d, J=7.5 Hz, 1H), 8.35-8.43 (m, 1H).

### Intermediate 5

2-(2-Chloro-6-fluorophenyl)-2-oxoethyl 5-amino-1-methyl-1H-pyrazole-4-carboxylate

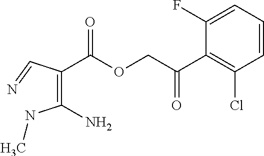 [SMILES: CN/C(N)=C(\C=N)C(=O)OCC(=O)c1c(F)cccc1Cl]

The titled intermediate was prepared by the reaction of 5-amino-1-methyl-1H-pyrazole-4-carboxylic acid (650 mg, 4.60 mmol) with 2-bromo-1-(2-chloro-6-fluorophenyl)ethanone (1.3 g, 5.52 mmol) using potassium fluoride (401 mg, 6.90 mmol) in dry DMF (6.5 mL) as per the procedure described in Step 4 of Intermediate 1 to yield 670 mg of the product as a solid. ¹H NMR (300 MHz, CDCl₃): δ 3.63 (s, 3H), 5.17 (br s, 2H), 5.51 (s, 2H), 7.08 (t, J=8.7 Hz, 1H), 7.21-7.28 (m, 1H), 7.30-7.45 (m, 1H), 7.62 (s, 1H).

### Intermediate 6

2-(2-Chlorophenyl)-2-oxoethyl 5-amino-1-(2,2,2-trifluoroethyl)-1H-pyrazole-4-carboxylate

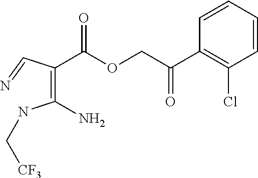 [SMILES: N=C/C(C(=O)OCC(=O)c1ccccc1Cl)=C(/N)NCC(F)(F)F]

### Step 1: Ethyl 5-amino-1-(2,2,2-trifluoroethyl)-1H-pyrazole-4-carboxylate

The titled intermediate was prepared by the reaction of ethyl 2-cyano-3-(dimethylamino)prop-2-enoate (2.5 g, 14.86 mmol) and (2,2,2-trifluoroethyl)hydrazine (1.69 g, 14.86 mmol) in dry ethanol (25 mL) as per the procedure described in Step 2 of Intermediate 1 to afford 1.42 g of the product as a solid. ¹H NMR (300 MHz, CDCl₃): δ 1.34 (t, J=7.5 Hz, 3H), 4.28 (q, J=6.6 Hz, 2H), 4.56 (q, J=8.7 Hz, 2H), 6.20 (br s, 2H), 7.70 (s, 1H).

### Step 2: 5-Amino-1-(2,2,2-trifluoroethyl)-1H-pyrazole-4-carboxylic acid

The titled intermediate was prepared by the ester hydrolysis of Step 1 intermediate (1.4 g, 5.902 mmol) using aqueous solution of potassium hydroxide (2.0 M, 6 mL, 8.853 mmol) as per the procedure described in Step 3 of Intermediate 1 to yield 680 mg of the product as a solid. ¹H NMR (300 MHz, DMSO-d₆): δ 4.90 (q, J=8.7 Hz, 2H), 6.56 (s, 2H), 7.51 (s, 1H), 11.90 (s, 1H).

### Step 3: 2-(2-Chlorophenyl)-2-oxoethyl 5-amino-1-(2,2,2-trifluoroethyl)-1H-pyrazole-4-carboxylate

The titled intermediate was prepared by the reaction of Step 2 intermediate (650 mg, 3.10 mmol) with 2-bromo-1-(2-chlorophenyl)ethanone (724 mg, 3.10 mmol) using potassium fluoride (270 mg, 4.66 mmol) in dry DMF (6.5 mL) as per the procedure described in Step 4 of Intermediate 1 to yield 410 mg of the product as a solid. ¹H NMR (300 MHz, DMSO-d₆): δ 4.93 (q, J=8.7 Hz, 2H), 5.34 (s, 2H), 6.75 (s, 2H), 7.44-7.52 (m, 1H), 7.55-7.63 (m, 3H), 7.79 (d, J=7.2 Hz, 1H).

### Intermediate 7

2-(2,6-Difluorophenyl)-2-oxoethyl 5-amino-1-(4-fluorophenyl)-1H-pyrazole-4-carboxylate

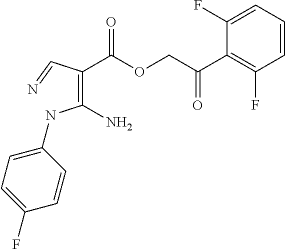 [SMILES: N=C/C(C(=O)OCC(=O)c1c(F)cccc1F)=C(/N)Nc1ccc(F)cc1]

### Step 1: Ethyl (2E)-2-cyano-3-ethoxyprop-2-enoate

To a stirred solution of ethyl cyanoacetate (10.0 g, 88.40 mmol) in acetic anhydride (100 mL) was added triethyl orthoformate (16.7 mL, 97.24 mmol) at RT. The reaction mixture was heated to 90° C. for 18 h. The reaction mixture was cooled to RT, solvent was evaporated under reduced pressure and the obtained product was purified by silica gel column chromatography to yield 8.0 g of the titled product as a solid. ¹H NMR (300 MHz, CDCl₃): δ 1.30 (t, J=7.2 Hz, 3H), 1.42 (t, J=7.2 Hz, 3H), 4.20-4.36 (m, 4H), 7.99 (s, 1H).

### Step 2: Ethyl 5-amino-1-(4-fluorophenyl)-1H-pyrazole-4-carboxylate

To a stirred solution of Step 1 intermediate (2.0 g, 10.14 mmol) in ethanol (20 mL), 4-fluorophenylhydrazine hydrochloride (1.97 g, 12.17 mmol) was added at RT and the reaction mixture was stirred overnight at 110° C. The rection mixture was cooled to RT, solvent were evaporated under reduced pressure and the residue was basified with saturated aqueous sodium bicarbonate solution till pH 9-10. The mixture was extracted with ethyl acetate (100 mL×2). The organic layer was dried over anhydrous sodium sulfate and concentrated under reduced pressure. The obtained product was purified by silica gel column chromatography to afford 2.65 g of the titled product as a solid. ¹H NMR (300 MHz, DMSO-d₆): δ 1.26 (t, J=7.2 Hz, 3H), 4.21 (q, J=7.2 Hz, 2H), 6.31 (s, 2H), 7.37 (t, J=8.1 Hz, 2H), 7.54-7.57 (m, 2H), 7.69 (s, 1H); APCI (m/z) 250 (M+H)⁺.

### Step 3: 5-Amino-1-(4-fluorophenyl)-1H-pyrazole-4-carboxylic acid

To a stirred solution of Step 2 intermediate (2.6 g, 10.42 mmol) in isopropyl alcohol (35 mL) was added potassium hydroxide (880 mg, 15.62 mmol) at RT. The mixture was stirred at 80° C. for 5 h. The solvent was evaporated under reduced pressure and the residue was acidified with nitric acid till pH 2-3. The precipitated solid was filtered, washed with water (40 mL×2) and dried under vacuum to yield 1.80 g of the titled product as a solid. ¹H NMR (300 MHz, DMSO-d₆): δ 6.27 (s, 2H), 7.36 (t, J=8.4 Hz, 2H), 7.54-7.60 (m, 2H), 7.66 (s, 1H), 12.07 (br s, 1H); APCI (m/z) 220 (M−H)⁻.

### Step 4: 2-(2,6-Difluorophenyl)-2-oxoethyl 5-amino-1-(4-fluorophenyl)-1H-pyrazole-4-carboxylate

The reaction of Step 3 intermediate (800 mg, 3.62 mmol) with 2-bromo-1-(2,6-difluorophenyl)ethanone (1.02 g, 4.34 mmol) using potassium fluoride (316 mg, 5.42 mmol) in dry DMF (8 mL) as per the procedure described in Step 4 of Intermediate 1 yielded 1.05 mg of the product as a solid. ¹H NMR (300 MHz, DMSO-d₆): δ 5.27 (s, 2H), 6.43 (s, 2H), 7.28 (t, J=7.8 Hz, 4H), 7.38 (t, J=8.1 Hz, 2H), 7.56-7.75 (m, 2H); ESI (m/z) 374 (M−H)⁻.

### Intermediate 8

2-(2-Chlorophenyl)-2-oxoethyl 5-amino-1-(4-fluorophenyl)-1H-pyrazole-4-carboxylate

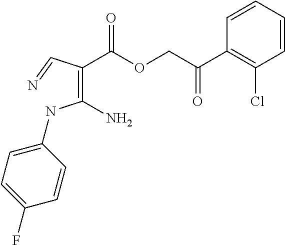 [SMILES: N=C/C(C(=O)OCC(=O)c1ccccc1Cl)=C(/N)Nc1ccc(F)cc1]

The titled compound was prepared by the reaction of 5-amino-1-(4-fluorophenyl)-1H-pyrazole-4-carboxylic acid (800 mg, 3.62 mmol) and 2-bromo-1-(2-chlorophenyl)ethanone (1.0 g, 4.29 mmol) in the presence of potassium fluoride (315 mg, 5.41 mmol) in dry DMF (8 mL) as per the procedure described in Step 4 of Intermediate 1 to obtain 1.05 g of the product as a solid.

¹H NMR (300 MHz, DMSO-d₆): δ 5.39 (s, 2H), 6.43 (s, 2H), 7.38 (t, J=8.7 Hz, 2H), 7.50-7.60 (m, 5H), 7.76-7.83 (m, 2H); APCI (m/z) 372 (M−H)⁻.

### Intermediate 9

2-(2-Chlorophenyl)-2-oxoethyl 5-amino-1,3-dimethyl-1H-pyrazole-4-carboxylate

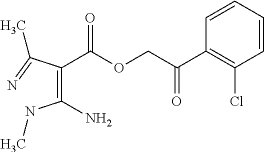 [SMILES: CN/C(N)=C(\C(C)=N)C(=O)OCC(=O)c1ccccc1Cl]

### Method-I:

### Step 1: Ethyl-2-cyano-3-(dimethylamino)but-2-enoate

The titled intermediate was prepared by the reaction of ethyl cyanoacetate (3.0 g, 26.52 mmol) with N,N′-dimethylformamide dimethyl acetal (5.0 mL, 34.47 mmol) in the presence of methanol (30 mL) as per the procedure described in Step 1 of Intermediate 1 to yield 5.1 g of the product as oil. ¹H NMR (300 MHz, CDCl₃): δ 1.32 (t, J=6.9 Hz, 3H), 2.49 (s, 3H), 3.11 (s, 3H), 3.26 (s, 3H), 4.18 (q, J=7.2 Hz, 2H).

### Step 2: Ethyl 5-amino-1,3-dimethyl-1H-pyrazole-4-carboxylate

The titled intermediate was prepared by the reaction of Step 1 intermediate (5.0 g, 27.43 mmol) with methyl hydrazine (1.5 mL, 27.43 mmol) using dry ethanol (50 mL) as per the procedure described in Step 2 of Intermediate 1 to yield 1.66 g of the product as a solid. ¹H NMR (300 MHz, DMSO-d₆): δ 1.34 (t, J=7.2 Hz, 3H), 2.33 (s, 3H), 3.56 (s, 3H), 4.27 (q, J=7.2 Hz, 2H), 5.07 (s, 2H).

### Step 3: 5-Amino-1,3-dimethyl-1H-pyrazole-4-carboxylic acid

The titled intermediate was prepared by the ester hydrolysis of Step 2 intermediate (1.6 g, 8.73 mmol) using aqueous solution of potassium hydroxide (2 M, 8.7 mL, 13.10 mmol) in ethanol (9.0 mL) as per the procedure described in Step 3 of Intermediate 1 to yield 563 mg of the product as a solid. ¹H NMR (300 MHz, CDCl₃): δ 2.12 (s, 3H), 3.43 (s, 3H), 6.09 (s, 2H), 11.69 (s, 1H).

### Step 4: 2-(2-Chlorophenyl)-2-oxoethyl 5-amino-1,3-dimethyl-1H-pyrazole-4-carboxylate

The titled intermediate was prepared by the reaction of Step 3 intermediate (550 mg, 3.54 mmol) with 2-bromo-1-(2-chlorophenyl)ethanone (826 mg, 3.54 mmol) using potassium fluoride (308 mg, 5.31 mmol) in dry DMF (6.0 mL) as per the procedure described in Step 4 of Intermediate 1 to yield 604 mg of the product as a solid. ¹H NMR (300 MHz, DMSO-d₆): δ 2.12 (s, 3H), 3.46 (s, 3H), 5.30 (s, 2H), 6.25 (s, 2H), 7.45-7.53 (m, 1H), 7.55-7.64 (m, 2H), 7.77 (d, J=7.8 Hz, 1H).

### Method-II:

### Step 1: (E)-ethyl 2-cyano-3-ethoxybut-2-enoate

The title intermediate was prepared by heating mixture of ethyl cyano acetate (50.0 g, 442 mmol) and triethylortho acetate (86.0 g, 530 mmol) at 110° C. for 2 h. The ethanol formed in the reaction was distilled out under reduced pressure and triethylortho acetate (86.0 g, 530 mmol) was added and mixture was heated at 130° C. for 4 h. The obtained product was purified by column chromatography using pet ether-ethyl acetate (90:10) to give 40 g of the titled product as an oil. ¹H NMR (300 MHz, CDCl₃): δ 1.32 (t, J=6.9 Hz, 3H), 1.43 (t, J=7.2 Hz, 3H), 2.61 (s, 3H), 3.11 (s, 3H), 4.15-4.35 (m, 4H).

### Step 2: Ethyl 5-amino-1,3-dimethyl-1H-pyrazole-4-carboxylate

The title intermediate was prepared by heating mixture of step 1 intermediate (17.0 g, 92.79 mmol) with methyl hydrazine sulfate (13.3 g, 92.79 mmol) using N,N′ diisopropylethyl amine (31.7 ml, 185.53 mmol) in dry ethanol (175 mL) at reflux temperature for overnight. The excess of ethanol was evaporated under reduced pressure. The residue was basified with aqueous saturated sodium bicarbonate solution (100 mL) and extracted with ethyl acetate (150 mL×3). The organic layer was dried over anhydrous sodium sulfate and concentrated under reduced pressure. The residue was purified by silica gel column chromatography to yield 14.1 of the product as a solid; ¹H NMR (300 MHz, DMSO-d₆): δ 1.34 (t, J=7.2 Hz, 3H), 2.33 (s, 3H), 3.56 (s, 3H), 4.27 (q, J=7.2 Hz, 2H), 5.07 (s, 2H).

### Step 3: 5-Amino-1,3-dimethyl-1H-pyrazole-4-carboxylic acid

To a stirred solution of step-2 intermediate (1.6 g, 8.734 mmol) in ethanol (8.7 mL), aqueous solution of potassium hydroxide (2.0 M, 8.7 mL, 13.101 mmol) was added and the reaction mixture was refluxed overnight. The reaction mixture was cooled to RT, concentrated under reduced pressure. The residue was stirred in 1.0 N citric acid (80 mL). The solid precipitated was filtered and dried to yield 560 mg of the desired product. ¹H NMR (300 MHz, CDCl₃): δ 2.12 (s, 3H), 3.43 (s, 3H), 6.09 (s, 2H), 11.69 (s, 1H).

### Step 4: 2-(2-Chlorophenyl)-2-oxoethyl 5-amino-1,3-dimethyl-1H-pyrazole-4-carboxylate

The titled intermediate was prepared by the reaction of Step 3 intermediate (550 mg, 3.54 mmol) with 2-bromo-1-(2-chlorophenyl)ethanone (826 mg, 3.54 mmol) using potassium fluoride (308 mg, 5.31 mmol) in dry DMF (6.0 mL) as per the procedure described in Step 4 of Intermediate 1 to yield 604 mg of the product as a solid. ¹H NMR (300 MHz, DMSO-d₆): δ 2.12 (s, 3H), 3.46 (s, 3H), 5.30 (s, 2H), 6.25 (s, 2H), 7.45-7.53 (m, 1H), 7.55-7.64 (m, 2H), 7.77 (d, J=7.8 Hz, 1H).

### Intermediate 10

2-(2,4-Dichlorophenyl)-2-oxoethyl 5-amino-1,3-dimethyl-1H-pyrazole-4-carboxylate

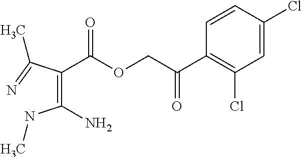 [SMILES: CN/C(N)=C(\C(C)=N)C(=O)OCC(=O)c1ccc(Cl)cc1Cl]

The titled compound was prepared by the reaction of 5-amino-1,3-dimethyl-1H-pyrazole-4-carboxylic acid (700 mg, 4.51 mmol) with 2-bromo-1-(2,4-dichlorophenyl)ethanone (1.2 g, 4.51 mmol) using potassium fluoride (393 mg, 6.76 mmol) in dry DMF (7.0 mL) as per the procedure described in Step 4 of Intermediate 1 to yield 716 mg of the product as a solid. ¹H NMR (300 MHz, DMSO-d₆): δ 2.12 (s, 3H), 3.46 (s, 3H), 5.29 (s, 2H), 6.25 (s, 2H), 7.60 (d, J=8.1 Hz, 1H), 7.75-7.86 (m, 2H).

### Intermediate 11

2-(2-Chloro-4-fluorophenyl)-2-oxoethyl 5-amino-1,3-dimethyl-1H-pyrazole-4-carboxylate

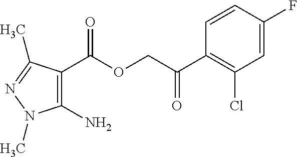 [SMILES: Cc1nn(C)c(N)c1C(=O)OCC(=O)c1ccc(F)cc1Cl]

The titled compound was prepared by the reaction of 5-amino-1,3-dimethyl-1H-pyrazole-4-carboxylic acid (600 mg, 3.86 mmol) with 2-bromo-1-(2-chloro-4-fluorophenyl)ethanone (1.16 g, 4.63 mmol) using potassium fluoride (336 mg, 5.79 mmol) in dry DMF (6.0 mL) as per the procedure described in Step 4 of Intermediate 1 to yield 743 mg of the product as a solid. ¹H NMR (300 MHz, CDCl₃): δ 2.31 (s, 3H), 3.57 (s, 3H), 5.13 (br s, 2H), 5.32 (s, 2H), 7.09 (t, J=9.0 Hz, 1H) 7.19 (d, J=8.1 Hz, 1H), 7.74 (t, J=8.7 Hz, 1H).

### Intermediate 12

2-(2,6-Difluorophenyl)-2-oxoethyl 5-amino-1,3-dimethyl-1H-pyrazole-4-carboxylate

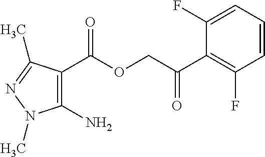 [SMILES: Cc1nn(C)c(N)c1C(=O)OCC(=O)c1c(F)cccc1F]

The titled compound was prepared by the reaction of 5-amino-1,3-dimethyl-1H-pyrazole-4-carboxylic acid (600 mg, 3.86 mmol) with 2-bromo-1-(2,6-difluorophenyl)ethanone (1.09 g, 4.63 mmol) using potassium fluoride (337 mg, 5.80 mmol) in dry DMF (6.0 mL) as per the procedure described in Step 4 of Intermediate 1 to yield 825 mg of the product as a solid. ¹H NMR (300 MHz, DMSO-d₆): δ 2.11 (s, 3H), 3.45 (s, 3H), 5.18 (s, 2H), 6.25 (s, 2H), 7.26 (t, J=8.4 Hz, 2H), 7.64-7.69 (m, 1H).

### Intermediate 13

2-(2,4-Difluorophenyl)-2-oxoethyl 5-amino-1,3-dimethyl-1H-pyrazole-4-carboxylate

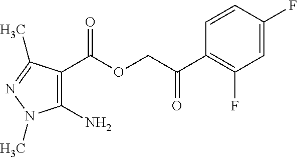 [SMILES: Cc1nn(C)c(N)c1C(=O)OCC(=O)c1ccc(F)cc1F]

The titled compound was prepared by the reaction of 5-amino-1,3-dimethyl-1H-pyrazole-4-carboxylic acid (500 mg, 3.22 mmol) with 1-[(bromooxy)carbonyl]-2,4-difluorobenzene (757 mg, 3.22 mmol) using potassium fluoride (280 mg, 4.81 mmol) in dry DMF (5.0 mL) as per the procedure described in Step 4 of Intermediate 1 to yield 520 mg of the product as a solid. ¹H NMR (300 MHz, DMSO-d₆): δ 2.17 (s, 3H), 3.47 (s, 3H), 5.33 (s, 2H), 6.25 (s, 2H), 7.29 (t, J=8.1 Hz, 1H), 7.49 (t, J=8.7 Hz, 1H), 7.95-8.03 (m, 1H).

### Intermediate 14

2-(3,4-Dimethylphenyl)-2-oxoethyl 5-amino-1,3-dimethyl-1H-pyrazole-4-carboxylate

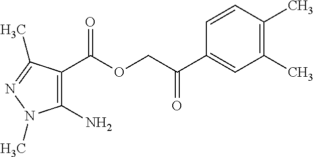 [SMILES: Cc1ccc(C(=O)COC(=O)c2c(C)nn(C)c2N)cc1C]

The titled compound was prepared by the reaction of 5-amino-1,3-dimethyl-1H-pyrazole-4-carboxylic acid (600 mg, 3.86 mmol) with 2-bromo-1-(3,4-dimethylphenyl)ethanone (1.05 g, 4.64 mmol) using potassium fluoride (337 mg, 5.80 mmol) in dry DMF (6.0 mL) as per the procedure described in Step 4 of Intermediate 1 to yield 730 mg of the product as a solid. ¹H NMR (300 MHz, CDCl₃): δ 2.38 (s, 9H), 3.57 (s, 3H), 5.23 (br s, 2H), 5.46 (s, 2H), 7.23 (s, 1H), 7.67-7.72 (m, 2H).

### Intermediate 15

2-[3-Fluoro-4-(trifluoromethoxy)phenyl]-2-oxoethyl 5-amino-1,3-dimethyl-1H-pyrazole-4-carboxylate

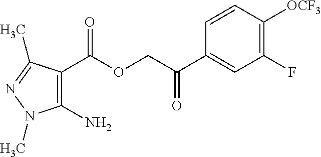 [SMILES: Cc1nn(C)c(N)c1C(=O)OCC(=O)c1ccc(OC(F)(F)F)c(F)c1]

The titled compound was prepared by the reaction of 5-amino-1,3-dimethyl-1H-pyrazole-4-carboxylic acid (500 mg, 3.22 mmol) with 2-bromo-1-[3-fluoro-4-(trifluoromethoxy)phenyl]ethanone (878 mg, 2.91 mmol) using potassium fluoride (280 mg, 4.81 mmol) in dry DMF (5.0 mL) as per the procedure described in Step 4 of Intermediate 1 to yield 710 mg of the product as a solid. ¹H NMR (300 MHz, DMSO-d₆): δ 2.17 (s, 3H), 3.50 (s, 3H), 5.53 (s, 2H), 6.26 (s, 2H), 7.71-7.77 (m, 1H), 8.13 (d, J=7.2 Hz, 2H).

### Intermediate 16

2-(3,4-Difluorophenyl)-2-oxoethyl 5-amino-1,3-dimethyl-1H-pyrazole-4-carboxylate

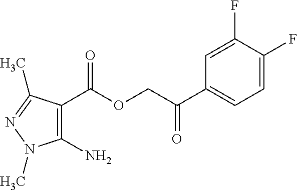 [SMILES: Cc1nn(C)c(N)c1C(=O)OCC(=O)c1ccc(F)c(F)c1]

The titled compound was prepared by the reaction of 5-amino-1,3-dimethyl-1H-pyrazole-4-carboxylic acid (600 mg, 3.86 mmol) with 2-bromo-1-(3,4-difluorophenyl)ethanone (1.1 g, 4.63 mmol) using potassium fluoride (336 mg, 5.79 mmol) in dry DMF (6.0 mL) as per the procedure described in Step 4 of Intermediate 1 to yield 698 mg of the product as a solid. ¹H NMR (300 MHz, DMSO-d₆): δ 2.17 (s, 3H), 3.47 (s, 3H), 5.50 (s, 2H), 6.26 (s, 2H), 7.60-7.70 (m, 1H), 7.85-7.90 (m, 1H), 8.07 (t, J=9.3 Hz, 1H); ESI (m/z) 310 (M+H)⁺.

Intermediate 17

2-(2-Chloro-4-methoxyphenyl)-2-oxoethyl 5-amino-1,3-dimethyl-1H-pyrazole-4-carboxylate

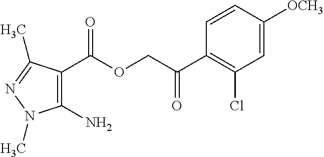 [SMILES: COc1ccc(C(=O)COC(=O)c2c(C)nn(C)c2N)c(Cl)c1]

The titled compound was prepared by the reaction of 5-amino-1,3-dimethyl-1H-pyrazole-4-carboxylic acid (500 mg, 3.22 mmol) in dry DMF (5 mL) were added potassium fluoride (280 mg, 4.82 mmol) and 2-bromo-1-(2-chloro-4-methoxyphenyl)ethanone (847 mg, 3.22 mmol) as per the procedure described in Step 4 of Intermediate 1 to afford 690 mg of the compound as a solid. ¹H NMR (300 MHz, DMSO-d₆): δ 2.14 (s, 3H), 3.46 (s, 3H), 3.95 (s, 3H), 5.31 (s, 2H), 6.25 (br s, 2H), 7.05 (d, J=8.1 Hz, 1H), 7.15 (s, 1H), 7.85 (d, J=8.4 Hz, 1H).

### Intermediate 18

2-(2-Fluoro-4-methoxyphenyl)-2-oxoethyl 5-amino-1,3-dimethyl-1H-pyrazole-4-carboxylate

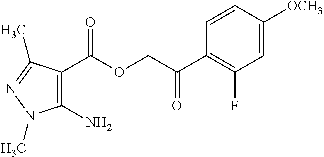 [SMILES: COc1ccc(C(=O)COC(=O)c2c(C)nn(C)c2N)c(F)c1]

The titled intermediate was prepared by the reaction of 2-bromo-1-(2-fluoro-4-methoxyphenyl)ethanone (1.52g, 6.18 mmol) with 5-amino-1,3- dimethyl-1H-pyrazole-4-carboxylic acid (800 mg, 5.15 mmol) using potassium fluoride (449 mg, 7.73 mmol) in dry DMF (8.0 mL) as per the procedure described in Step 4 of Intermediate 1 to yield 670 mg of the product as a solid. ¹H NMR (300 MHz, DMSO-d₆): δ 2.17 (s, 3H), 3.47 (s, 3H), 3.87 (s, 3H), 5.29 (br s, 2H), 6.26 (br s, 2H), 6.92-7.05 (m, 2H), 7.86 (t, J=8.1 Hz, 1H); ESI (m/z) 322 (M+H)⁺.

### Intermediate 19

2-(2,5-Dichlorophenyl)-2-oxoethyl 5-amino-1,3-dimethyl-1H-pyrazole-4-carboxylate

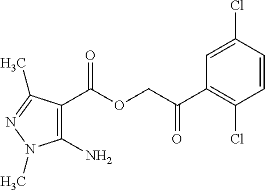 [SMILES: Cc1nn(C)c(N)c1C(=O)OCC(=O)c1cc(Cl)ccc1Cl]

The titled intermediate was prepared by the reaction of 2-bromo-1-(2,5-dichlorophenyl)ethanone (860 mg, 3.22 mmol) with 5-amino-1,3-dimethyl-1H-pyrazole-4-carboxylic acid (500 mg, 3.22 mmol) using potassium fluoride (280 mg, 4.82 mmol) in dry DMF (5.0 mL) as per the procedure described in Step 4 of Intermediate 1 to yield 510 mg of the product as a solid. ¹H NMR (300 MHz, DMSO-d₆): δ 2.12 (s, 3H), 3.45 (s, 3H), 5.29 (s, 2H), 6.26 (br s, 2H), 7.60-7.66 (m, 2H), 7.87 (s, 1H); ESI (m/z) 342 (M)⁺.

### Intermediate 20

2-[2-Fluoro-4-(trifluoromethyl)phenyl]-2-oxoethyl 5-amino-1,3-dimethyl-1H-pyrazole-4-carboxylate

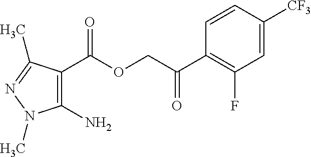 [SMILES: Cc1nn(C)c(N)c1C(=O)OCC(=O)c1ccc(C(F)(F)F)cc1F]

The titled compound was prepared by the reaction of 2-bromo-1-[2-fluoro-4-(trifluoromethyl)phenyl]ethanone (881 mg, 3.09 mmol) with 5-amino-1,3-dimethyl-1H-pyrazole-4-carboxylic acid (400 mg, 2.57 mmol) using potassium fluoride (224 mg, 3.86 mmol) in dry DMF (4.0 mL) as per the procedure described in Step 4 of Intermediate 1 to afford 485 mg of the product as a solid. ¹H NMR (300 MHz, DMSO-d₆): δ 2.16 (s, 3H), 3.47 (s, 3H), 5.38 (s, 2H), 6.28 (br s, 2H), 7.77 (d, J=8.4 Hz, 1H), 7.96 (d, J=10.8 Hz, 1H), 8.05-8.10 (m, 1H); ESI (m/z) 360 (M+H)⁺.

### Intermediate 21

2-[3-Fluoro-4-(trifluoromethyl)phenyl]-2-oxoethyl 5-amino-1,3-dimethyl-1H-pyrazole-4-carboxylate

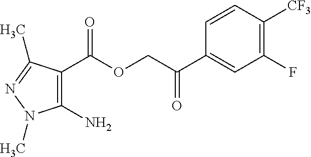 [SMILES: Cc1nn(C)c(N)c1C(=O)OCC(=O)c1ccc(C(F)(F)F)c(F)c1]

The titled compound was prepared by the reaction of 5-amino-1,3-dimethyl-1H-pyrazole-4-carboxylic acid (500 mg, 3.22 mmol and 2-bromo-1-[3-fluoro-4-(trifluoromethyl)phenyl]ethanone (912 mg, 3.22 mmol) using potassium fluoride (280 mg, 4.83 mmol) in dry DMF (5.0 mL) at RT as per the procedure described in Step 4 of Intermediate 1 to afford 613 mg of the product as a solid. ¹H NMR (300 MHz, DMSO-d₆): δ 2.17 (s, 3H), 3.47 (s, 3H), 5.55 (s, 2H), 6.28 (br s, 2H), 7.95-8.00 (m, 2H), 8.02-8.12 (m, 1H); ESI (m/z) 360 (M+H)⁺.

### Intermediate 22

2-(2-Chloro-5-methoxyphenyl)-2-oxoethyl 5-amino-1,3-dimethyl-1H-pyrazole-4-carboxylate

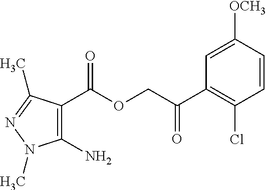 [SMILES: COc1ccc(Cl)c(C(=O)COC(=O)c2c(C)nn(C)c2N)c1]

The titled compound was prepared by the reaction of 2-bromo-1-(2-chloro-5-methoxyphenyl)ethanone (843 mg, 3.20 mmol) and 5-amino-1,3-dimethyl-1H-pyrazole-4-carboxylic acid (350 mg, 2.25 mmol) in the presence of potassium fluoride (280 mg, 4.83 mmol) in dry DMF (3.5 mL) at RT as per the procedure described in Step 4 of Intermediate 1 to afford 387 mg of the compound as a solid. ¹H NMR (300 MHz, DMSO-d₆): δ 2.12 (s, 3H), 3.45 (s, 3H), 3.80 (s, 3H), 5.29 (s, 2H), 6.25 (br s, 2H), 7.13-7.17 (m, 1H), 7.28 (s, 1H), 7.47 (d, J=8.7 Hz, 1H).

### Intermediate 23

2-[4-Chloro-3-(trifluoromethyl)phenyl]-2-oxoethyl 5-amino-1,3-dimethyl-1H-pyrazole-4-carboxylate

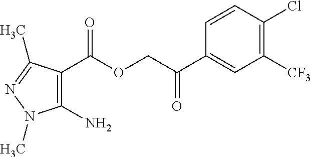 [SMILES: Cc1nn(C)c(N)c1C(=O)OCC(=O)c1ccc(Cl)c(C(F)(F)F)c1]

The titled intermediate was prepared by the reaction of 5-amino-1,3-dimethyl-1H-pyrazole-4-carboxylic acid (413 mg, 2.66 mmol) with 2-bromo-1-[4-chloro-3-(trifluoromethyl)phenyl]ethanone (800 mg, 2.66 mmol) using potassium fluoride (231 mg, 3.99 mmol) in dry DMF (4.0 mL) as per the procedure described in Step 4 of Intermediate 1 to yield 513 mg of the product as a solid. ¹H NMR (300 MHz, DMSO-d₆): δ 2.17 (s, 3H), 3.46 (s, 3H), 5.56 (s, 2H), 6.26 (s, 2H), 7.95 (d, J=8.1 Hz, 1H), 8.25-8.31 (m, 2H); ESI (m/z) 376 (M+H)⁺.

### Intermediate 24

2-(4-Chloro-2-fluorophenyl)-2-oxoethyl 5-amino-1,3-dimethyl-1H-pyrazole-4-carboxylate

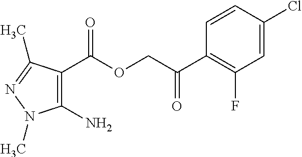 [SMILES: Cc1nn(C)c(N)c1C(=O)OCC(=O)c1ccc(Cl)cc1F]

The titled intermediate was prepared by the reaction of 5-amino-1,3-dimethyl-1H-pyrazole-4-carboxylic acid (600 mg, 3.86 mmol) with 2-bromo-1-(4-chloro-2-fluorophenyl)ethanone (1.16 g, 4.63 mmol) using potassium fluoride (335 mg, 0.33 mmol) in dry DMF (6.0 mL) as per the procedure described in Step 4 of Intermediate 1 to yield 850 mg of the product as a solid. ¹H NMR (300 MHz, CDCl₃): δ 2.36 (s, 3H), 3.56 (s, 3H), 5.09 (s, 2H), 5.34 (d, J=3.9 Hz, 2H), 7.26 (t, J=8.7 Hz, 2H), 7.95 (t, J=7.8 Hz, 1H).

### Intermediate 25

2-(2-Chlorophenyl)-2-oxoethyl 5-amino-1-ethyl-3-methyl-1H-pyrazole-4-carboxylate

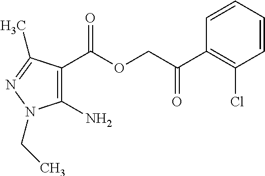 [SMILES: CCn1nc(C)c(C(=O)OCC(=O)c2ccccc2Cl)c1N]

### Step 1: Ethyl 5-amino-1-ethyl-3-methyl-1H-pyrazole-4-carboxylate

The titled compound was prepared by the reaction of ethyl 2-cyano-3-(dimethylamino)but-2-enoate (2.5 g, 13.71 mmol) with ethyl hydrazine oxalate (2.0 g, 13.71 mmol) using triethylamine (3.8 mL, 27.42 mmol) in dry ethanol (25 mL) as per the procedure described in Step 2 of Intermediate 1 to yield 980 mg of the product as a solid. ¹H NMR (300 MHz, CDCl₃): δ 1.35 (t, J=7.2 Hz, 6H), 2.34 (s, 3H), 3.86 (q, J=7.2 Hz, 2H), 4.26 (q, J=7.2 Hz, 2H), 5.08 (s, 2H).

### Step 2: 5-Amino-1-ethyl-3-methyl-1H-pyrazole-4-carboxylic acid

The titled compound was prepared by the ester hydrolysis of Step 1 intermediate (960 mg, 4.86 mmol) using aqueous solution of potassium hydroxide (1.25 M, 7.30 mmol) in ethanol (16 mL) as per the procedure described in Step 3 of Intermediate 1 to yield 720 mg of the product as a solid. ¹H NMR (300 MHz, DMSO-d₆): δ 1.18 (t, J=7.2 Hz, 3H), 2.14 (s, 3H), 3.83 (q, J=7.2 Hz, 2H), 6.12 (br s, 2H), 11.69 (br s, 1H).

### Step 3: 2-(2-Chlorophenyl)-2-oxoethyl 5-amino-1-ethyl-3-methyl-1H-pyrazole-4-carboxylate

The titled compound was prepared by the reaction of Step 2 intermediate (470 mg, 2.78 mmol) with 2-bromo-1-(2-chlorophenyl)ethanone (779 mg, 3.33 mmol) using potassium fluoride (243 mg, 4.17 mmol) in dry DMF (5.0 mL) as per the procedure described in Step 4 of Intermediate 1 to yield 720 mg of the product as a solid. ¹H NMR (300 MHz, CDCl₃): δ 1.38 (t, J=7.2 Hz, 3H), 2.30 (s, 3H), 3.88 (q, J=7.2 Hz, 2H), 5.08 (br s, 2H), 5.34 (s, 2H), 7.39-7.40 (m, 1H), 7.45-7.46 (m, 2H), 7.65 (d, J=7.2 Hz, 1H).

### Intermediate 26

2-(2-Chlorophenyl)-2-oxoethyl 5-amino-3-methyl-1-(2,2,2-trifluoroethyl)-1H-pyrazole-4-carboxylate

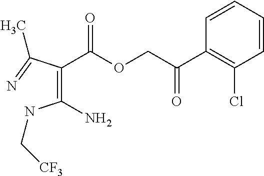 [SMILES: CC(=N)/C(C(=O)OCC(=O)c1ccccc1Cl)=C(/N)NCC(F)(F)F]

### Step 1: Ethyl 5-amino-3-methyl-1-(2,2,2-trifluoroethyl)-1H-pyrazole-4-carboxylate

The titled compound was prepared by the reaction of ethyl 2-cyano-3-(dimethylamino)but-2-enoate (2.5 g, 13.71 mmol) and (2,2,2-trifluoroethyl)hydrazine (70% in water, 2.2 g, 13.71 mmol) in ethanol (25 mL) as per the procedure described in Step 2 of Intermediate 1 to afford 1.12 g of the product as colorless oil. ¹H NMR (300 MHz, CDCl₃): δ 1.34 (t, J=7.5 Hz, 3H), 2.34 (s, 3H), 4.25 (q, J=6.6 Hz, 2H), 4.50 (q, J=8.7 Hz, 2H), 5.31 (br s, 2H).

### Step 2: 5-Amino-3-methyl-1-(2,2,2-trifluoroethyl)-1H-pyrazole-4-carboxylic acid

The titled compound was prepared by the ester hydrolysis of Step 1 intermediate (1.1 g, 4.37 mmol) using potassium hydroxide (367 mg, 6.56 mmol) in water and ethanol (1:1, 9.0 mL) as per the procedure described in Step 3 of Intermediate 1 to yield 720 mg of the product as a solid. ¹H NMR (300 MHz, CDCl₃): δ 2.35 (s, 3H), 4.28-4.35 (m, 2H), 5.28 (br s, 2H).

### Step 3: 2-(2-Chlorophenyl)-2-oxoethyl 5-amino-3-methyl-1-(2,2,2-trifluoroethyl)-1H-pyrazole-4-carboxylate.

The reaction of Step 2 intermediate (700 mg, 3.13 mmol) with 2-chlorophenyl bromide (731 mg, 3.13 mmol) using potassium fluoride (273 mg, 4.69 mmol) in dry DMF (7.0 mL) as per the procedure described in Step 4 of Intermediate 1 yielded 319 mg of the product as a solid. ¹H NMR (300 MHz, DMSO-d₆): δ 2.16 (s, 3H), 4.80-4.87 (m, 2H), 5.32 (s, 2H), 6.55 (br s, 1H), 6.69 (br s, 1H), 7.49-7.52 (m, 1H), 7.55-7.60 (m, 2H), 7.75-7.78 (m, 1H).

### Intermediate 27

2-(2-Chlorophenyl)-2-oxoethyl 5-amino-3-methyl-1-(propan-2-yl)-1H-pyrazole-4-carboxylate

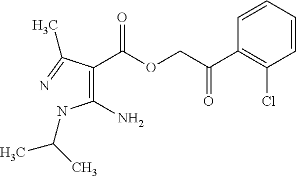 [SMILES: CC(=N)/C(C(=O)OCC(=O)c1ccccc1Cl)=C(/N)NC(C)C]

### Step 1: Ethyl 5-amino-3-methyl-1-(propan-2-yl)-1H-pyrazole-4-carboxylate

The titled compound was prepared by the reaction of ethyl 2-cyano-3-(dimethylamino)but-2-enoate (2.5 g, 13.71 mmol) with isopropyl hydrazine hydrochloride (1.51 g, 13.71 mmol) using triethylamine (3.82 mL, 27.42 mmol) in dry ethanol (50 mL) as per the procedure described in Step 2 of Intermediate 1 to yield 1.87 g of the product as oil. ¹H NMR (300 MHz, CDCl₃): δ 1.34 (t, J=7.2 Hz, 3H), 1.45 (d, J=6.9 Hz, 6H), 2.04 (s, 3H), 4.11-4.17 (m, 1H), 4.27 (q, J=7.2 Hz, 2H), 5.05 (s, 2H).

### Step 2: 5-Amino-3-methyl-1-(propan-2-yl)-1H-pyrazole-4-carboxylic acid

The titled compound was prepared by the ester hydrolysis of Step 1 intermediate (1.8 g, 8.520 mmol) using aqueous solution of potassium hydroxide (2.0 M, 8.5 mL, 10.21 mmol) in ethanol (8.5 mL) as per the procedure described in Step 3 of Intermediate 1 to yield 812 mg of the product as a solid. δ 1.25 (d, J=6.9 Hz, 6H), 2.15 (s, 3H), 4.33-4.38 (m, 1H), 6.12 (s, 2H), 11.66 (br s, 1H).

### Step 4: 2-(2-Chlorophenyl)-2-oxoethyl 5-amino-3-methyl-1-(propan-2-yl)-1H-pyrazole-4-carboxylate.

The titled compound was prepared by the reaction of Step 2 intermediate (800 mg, 4.36 mmol) with 2-bromo-1-(2-chlorophenyl)ethanone (1.01 g, 4.36 mmol) using potassium fluoride (380 mg, 6.54 mmol) in dry DMF (8.0 mL) as per the procedure described in Step 4 of Intermediate 1 to yield 1.03 g of the product as viscous oil. ¹H NMR (300 MHz, DMSO-d₆): δ 1.27 (d, J=6.3 Hz, 6H), 2.14 (s, 3H), 4.36-4.42 (m, 1H), 5.30 (s, 2H), 6.25 (s, 2H), 7.49-7.51 (m, 1H), 7.55-7.60 (m, 2H), 7.77 (d, J=7.2 Hz, 1H).

### Intermediate 28

2-(2,6-Difluorophenyl)-2-oxoethyl 5-amino-1-(4-methoxyphenyl)-3-methyl-1H-pyrazole-4-carboxylate

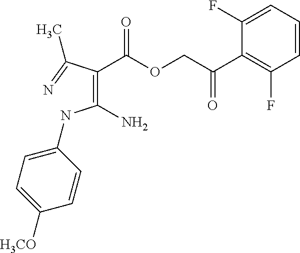 [SMILES: COc1ccc(N/C(N)=C(\C(C)=N)C(=O)OCC(=O)c2c(F)cccc2F)cc1]

### Step 1: Ethyl 5-amino-1-(4-methoxyphenyl)-3-methyl-1H-pyrazole-4-carboxylate

The titled compound was prepared by the reaction of ethyl-2-cyano-3-ethoxybut-2-enoate (2.0 g, 10.14 mmol) and 4-methoxyphenyl)hydrazine hydrochloride (2.28 g, 13.05 mmol) using triethylamine (1.84 mL, 13.05 mmol) in dry ethanol (20 mL) as per the procedure described in Step 2 of Intermediate 1 to afford 2.70 g of the product as a solid. ¹H NMR (300 MHz, CDCl₃): δ 1.36 (t, J=7.2 Hz, 3H), 2.40 (s, 3H), 3.84 (s, 3H), 4.30 (q, J=6.9 Hz, 2H), 5.23 (br s, 2H), 6.98 (d, J=9.3 Hz, 2H), 7.40 (t, J=8.7 Hz, 2H).

### Step 2: 5-Amino-1-(4-methoxyphenyl)-3-methyl-1H-pyrazole-4-carboxylic acid

The titled compound was prepared by the ester hydrolysis of Step 1 intermediate (2.65 g, 9.62 mmol) using potassium hydroxide (808 mg, 14.43 mmol) in water (12 mL) and ethanol (36 mL) as per the procedure described in Step 3 of Intermediate 1 to yield 1.81 g of the product as a solid. ¹H NMR (300 MHz, DMSO-d₆): δ 2.22 (s, 3H), 3.79 (s, 3H), 6.12 (s, 2H), 7.04 (d, J=8.7 Hz, 2H), 7.40 (d, J=8.7 Hz, 2H), 11.98 (br s, 1H).

### Step 3: 2-(2,6-Difluorophenyl)-2-oxoethyl 5-amino-1-(4-methoxyphenyl)-3-methyl-1H-pyrazole-4-carboxylate.

The reaction of Step 2 intermediate (600 mg, 3.61 mmol) with 2-bromo-1-(2,6-difluorophenyl)ethanone (685 mg, 2.91 mmol) using potassium fluoride (211 mg, 3.63 mmol) in dry DMF (6.0 mL) as per the procedure described in Step 4 of Intermediate 1 to afford 710 mg of the product as a solid. ¹H NMR (300 MHz, DMSO-d₆): δ 2.21 (s, 3H), 3.80 (s, 3H), 5.25 (s, 2H), 6.25 (s, 2H), 7.06 (d, J=8.7 Hz, 2H), 7.28 (t, J=8.4 Hz, 2H), 7.40 (d, J=8.7 Hz, 2H), 7.62-7.73 (m, 1H).

### Intermediate 29

2-(2-Chlorophenyl)-2-oxoethyl 5-amino-1-(pyridin-2-yl))-3-methyl-1H-pyrazole-4-carboxylate.

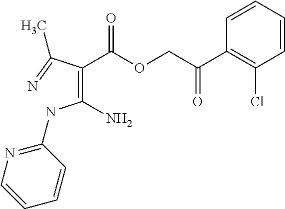 [SMILES: CC(=N)/C(C(=O)OCC(=O)c1ccccc1Cl)=C(/N)Nc1ccccn1]

### Step 1: Ethyl 5-amino-3-methyl-1-(pyridin-2-yl)-1H-pyrazole-4-carboxylate

The titled compound was prepared by the reaction of ethyl-2-cyano-3-ethoxybut-2-enoate (1.5 g, 8.19 mmol) and 2-hydrazinylpyridine (1.07 g, 9.82 mmol) using triethylamine (1.15 mL, 8.19 mmol) in dry ethanol (15 mL) as per the procedure described in Step 2 of Intermediate 1 to afford 2.02 g of the product as a solid. ¹H NMR (300 MHz, DMSO-d₆): δ 1.28 (t, J=7.2 Hz, 3H), 2.29 (s, 3H), 4.20 (q, J=7.2 Hz, 2H), 7.28 (t, J=6.9 Hz, 1H), 7.62 (br s, 2H), 7.81 (d, J=8.4 Hz, 1H), 7.94-7.99 (m, 1H), 8.42-8.44 (m, 1H); APCI (m/z) 247(M+H)⁺.

### Step 2: 5-Amino-3-methyl-1-(pyridin-2-yl)-1H-pyrazole-4-carboxylic acid

The titled compound was prepared by the ester hydrolysis of Step 1 intermediate (2.0 g, 8.12 mmol) using potassium hydroxide (682 mg, 12.18 mmol) in water (6.5 mL) and ethanol (27 mL) as per the procedure described in Step 3 of Intermediate 1 to yield 1.30 g of the product as a solid. ¹H NMR (300 MHz, DMSO-d₆): δ 2.28 (s, 3H), 7.28 (br s, 1H), 7.57 (br s, 2H), 7.80-7.85 (m, 1H), 7.95-7.97 (m, 1H), 8.44 (br s, 1H), 12.07 (br s, 1H); APCI (m/z) 219 (M+H)⁺.

### Step 3: 2-(2-Chlorophenyl)-2-oxoethyl 5-amino-3-methyl-1-(p yridin-2-yl)-1H-pyrazole-4-carboxylate.

The reaction of Step 2 intermediate (800 mg, 3.66 mmol) with 2-bromo-1-(2-chlorophenyl)ethanone (1.02 g, 4.39 mmol) using potassium fluoride (318 mg, 5.49 mmol) in dry DMF (8.0 mL) as per the procedure described in Step 4 of Intermediate 1 yielded 780 mg of the product as a solid. ¹H NMR (300 MHz, DMSO-d₆): δ 2.27 (s, 3H), 5.41 (br s, 2H), 7.29-7.33 (m, 1H), 7.51-7.55 (m, 1H), 7.56-7.60 (m, 2H), 7.75-7.85 (m, 2H), 7.95-8.00 (m, 1H), 8.44-8.46 (m, 1H); APCI (m/z) 371 (M+H)⁺.

### Intermediate 30

2-(2-Chlorophenyl)-2-oxoethyl 5-amino-1-(3,4-difluorophenyl)-3-methyl-1H-pyrazole-4-carboxylate

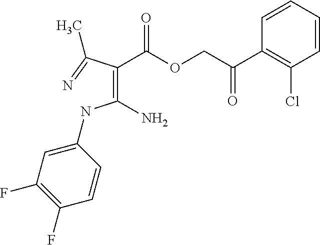 [SMILES: CC(=N)/C(C(=O)OCC(=O)c1ccccc1Cl)=C(/N)Nc1ccc(F)c(F)c1]

### Step 1: Ethyl 5-amino-1-(3,4-difluorophenyl)-3-methyl-1H-pyrazole-4-carboxylate

The titled compound was prepared by the reaction of (3,4-difluorophenyl)hydrazine (1.7 g, 13.64 mmol) ethyl (2Z)-2-cyano-3-methoxybut-2-enoate (1.9 g, 10.37 mmol) using triethylamine (1.87 mL, 27.42 mmol) in dry ethanol (20 mL) as per the procedure described in Step 2 of Intermediate 1 to yield 1.83 g of the product as oil. ¹H NMR (300 MHz, CDCl₃): δ 1.37 (t, J=7.5 Hz, 3H), 2.39 (s, 3H), 4.31 (q, J=7.5 Hz, 2H), 5.39 (br s, 2H), 7.27-7.32 (m, 2H), 7.42 (t, J=9.3 Hz, 1H); ESI (m/z) 282 (M+H)⁺.

### Step 2: 5-Amino-1-(3,4-difluorophenyl)-3-methyl-1H-pyrazole-4-carboxylic acid

The titled intermediate was prepared by the ester hydrolysis of Step 1 intermediate (1.8 g, 6.40 mmol) using aqueous solution of potassium hydroxide (626 mg, 11.18 mmol) in water (9.0 mL) as per the procedure described in Step 3 of Intermediate 1 to yield 1.5 g of the product as a solid. ESI (m/z) 254 (M+H)⁺.

### Step 3: 2-(2-Chlorophenyl)-2-oxoethyl 5-amino-1-(3,4-difluorophenyl)-3-methyl-1H-pyrazole-4-carboxylate

The reaction of Step 2 intermediate (600 mg, 2.37 mmol) with 2-bromo-1-(2-chlorophenyl)ethanone (553 mg, 2.35 mmol) using potassium fluoride (206 mg, 3.55 mmol) in dry DMF (6.0 mL) as per the procedure described in Step 4 of Intermediate 1 yielded 626 mg of the product as a solid. ¹H NMR (300 MHz, DMSO-d₆): δ 2.23 (s, 3H), 5.38 (s, 2H), 6.53 (s, 2H), 7.35-7.40 (m, 1H), 7.51-7.65 (m, 5H), 7.80 (d, J=7.2 Hz, 1H); ESI (m/z) 406 (M+H)⁺.

### Intermediate 31

2-(2,6-Difluorophenyl)-2-oxoethyl 5-amino-1-(4-fluorophenyl)-3-methyl-1H-pyrazole-4-carboxylate

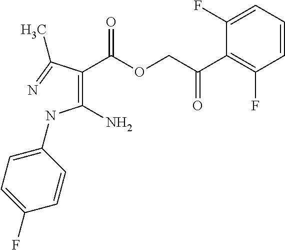 [SMILES: CC(=N)/C(C(=O)OCC(=O)c1c(F)cccc1F)=C(/N)Nc1ccc(F)cc1]

### Step 1: Ethyl 5-amino-1-(4-fluorophenyl)-3-methyl-1H-pyrazole-4-carboxylate

The titled compound was prepared by the reaction of ethyl-2-cyano-3-ethoxybut-2-enoate (2.0 g, 10.91 mmol) and (4-fluorophenyl)hydrazine hydrochloride (2.13 g, 13.09 mmol) using triethylamine (1.9 mL, 14.18 mmol) in dry ethanol (20 mL) as per the procedure described in Step 2 of Intermediate 1 to afford 2.68 g of the product as a solid. ¹H NMR (300 MHz, CDCl₃): δ 1.37 (t, J=7.5 Hz, 3H), 2.39 (s, 3H), 4.31 (q, J=7.5 Hz, 2H), 5.28 (br s, 2H), 7.18 (t, J=8.7 Hz, 2H), 7.46-7.52 (m, 2H); ESI (m/z) 264 (M+H)⁺.

### Step 2: 5-Amino-1-(4-fluorophenyl)-3-methyl-1H-pyrazole-4-carboxylic acid

The titled compound was prepared by the ester hydrolysis of Step 1 intermediate (2.6 g, 9.87 mmol) using potassium hydroxide (829 mg, 14.80 mmol) in water (12 mL) and ethanol (32 mL) as per the procedure described in Step 3 of Intermediate 1 to yield 2.01 g of the product as a solid. ¹H NMR (300 MHz, DMSO-d₆): δ 2.24 (s, 3H), 6.26 (s, 2H), 7.33 (t, J=9.0 Hz, 2H), 7.52-7.57 (m, 2H), 12.03 (br s, 1H); APCI (m/z) 236 (M+H)⁺.

### Step 3: 2-(2,6-Difluorophenyl)-2-oxoethyl 5-amino-1-(4-fluorophenyl)-3-methyl-1H-pyrazole-4-carboxylate.

The titled compound was prepared by the reaction of Step 2 intermediate (700 mg, 2.97 mmol) with 2,6-difluorophenacylbromide (701 mg, 3.36 mmol) in the presence of potassium fluoride (259 mg, 4.46 mmol) in DMF (7.0 mL) as per the procedure described in Step 4 of Intermediate 1 to yield 756 mg of the product as a solid. ¹H NMR (300 MHz, DMSO-d₆): δ 2.22 (s, 3H), 5.26 (s, 2H), 6.40 (s, 2H), 7.25-7.39 (m, 4H), 7.52-7.56 (m, 2H), 7.65-7.72 (m, 1H); ESI (m/z) 390 (M+H)⁺.

### Intermediate 32

2-(2-Chlorophenyl)-2-oxoethyl 5-amino-1-(4-fluorophenyl)-3-methyl-1H-pyrazole-4-carboxylate

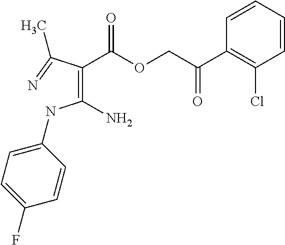 [SMILES: CC(=N)/C(C(=O)OCC(=O)c1ccccc1Cl)=C(/N)Nc1ccc(F)cc1]

The titled compound was prepared by the reaction of 5-amino-1-(4-fluorophenyl)-3-methyl-1H-pyrazole-4-carboxylic acid (800 mg, 3.40 mmol) with 2-bromo-1-(2-chlorophenyl)ethanone (793 mg, 3.40 mmol) using potassium fluoride (296 mg, 5.11 mmol) in dry DMF (8 mL) as per the procedure described in Step 4 of Intermediate 1 to yield 965 mg of the product as a solid. ¹H NMR (300 MHz, DMSO-d₆): δ 2.23 (s, 3H), 5.38 (s, 2H), 6.40 (s, 2H), 7.36 (t, J=9.3 Hz, 2H), 7.50-7.60 (m, 4H), 7.80 (d, J=7.5 Hz, 2H); ESI (m/z) 388 (M)⁺.

### Intermediate

2-(2-Chlorophenyl)-2-oxoethyl 5-amino-1-(3-fluorophenyl)-3-methyl-1H-pyrazole-4-carboxylate.

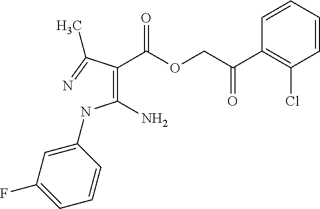 [SMILES: CC(=N)/C(C(=O)OCC(=O)c1ccccc1Cl)=C(/N)Nc1cccc(F)c1]

### Step 1: Ethyl 5-amino-1-(3-fluorophenyl)-3-methyl-1H-pyrazole-4-carboxylate

To a stirred solution of ethyl (2Z)-2-cyano-3-methoxybut-2-enoate (1.5 g, 8.18 mmol) in ethanol (15 mL) were added 3-fluorophenylhydrazine (1.6 g, 9.82 mmol) and triethylamine (1.5 mL, 10.64 mmol) at RT. The reaction mixture was refluxed for 18 h. The reaction mixture was cooled to RT and diluted with cold water (100 mL). The precipitated solid was filtered and dried under vacuum to obtain 1.82 g of the titled compound. ¹H NMR (300 MHz, CDCl₃): δ 1.37 (t, J=7.5 Hz, 3H), 2.40 (s, 3H), 4.31 (q, J=7.5 Hz, 2H), 5.41 (br s, 2H), 7.02-7.08 (m, 1H), 7.30-7.36 (m, 2H), 7.40-7.48 (m, 1H).

### Step 2: 5-Amino-1-(3-fluorophenyl)-3-methyl-1H-pyrazole-4-carboxylic acid

To a stirred solution of Step 1 intermediate (1.8 g, 6.83 mmol) in ethanol (22 mL) was added a solution of potassium hydroxide (574 mg, 10.25 mmol) in water (8.0 mL) at RT. The reaction mixture was refluxed overnight. The mixture was cooled to RT and the ethanol was recovered under reduced pressure. The concentrated aqueous mixture was acidified with 1 N citric acid till pH 2-3. The precipitated solid was filtered and dried under vacuum to yield 1.31 g of the titled product. ¹H NMR (300 MHz, DMSO-d₆): δ 2.23 (s, 3H), 6.43 (s, 2H), 7.20 (br s, 1H), 7.39-7.42 (m, 2H), 7.50-7.54 (m, 1H), 12.12 (br s, 1H); ESI (m/z) 236 (M+H)⁺.

### Step 3: 2-(2-Chlorophenyl)-2-oxoethyl 5-amino-1-(3-fluorophenyl)-3-methyl-1H-pyrazole-4-carboxylate

The titled compound was prepared by the reaction of Step 2 intermediate (800 mg, 3.40 mmol) with 2-chlorophenacylbromide (935 mg, 4.08 mmol) using potassium fluoride (296 mg, 5.10 mmol) in dry DMF (8.0 mL) as per the procedure described in Step 4 of Intermediate 1 to obtain 1.1 g of the product as oil. ¹H NMR (300 MHz, CDCl₃): δ 2.38 (s, 3H), 5.39 (s, 2H), 5.48 (br s, 2H), 7.10 (t, J=8.7 Hz, 1H), 7.32-7.41 (m, 3H), 7.42-7.48 (m, 3H), 7.67 (d, J=7.8 Hz, 1H); ESI (m/z) 388 (M)⁺.

### Intermediate 34

2-(2,6-Difluorophenyl)-2-oxoethyl 5-amino-1-(3-fluorophenyl)-3-methyl-1H-pyrazole-4-carboxylate

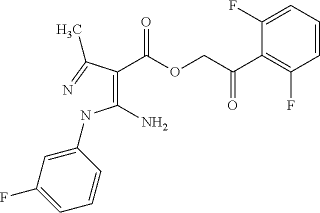 [SMILES: CC(=N)/C(C(=O)OCC(=O)c1c(F)cccc1F)=C(/N)Nc1cccc(F)c1]

The titled compound was prepared by the reaction of 5-amino-1-(3-fluorophenyl)-3-methyl-1H-pyrazole-4-carboxylic acid (500 mg, 1.97 mmol) with 2,6-difluorophenacylbromide (557 mg, 2.36 mmol) in the presence of potassium fluoride (172 mg, 2.96 mmol) at RT in DMF (5.0 mL) as per the procedure described in Step 4 of Intermediate 1 to yield 660 mg of the product as a solid. ¹H NMR (300 MHz, CDCl₃): δ 2.38 (s, 3H), 5.25 (s, 2H), 5.50 (br s, 2H), 7.01 (t, J=8.4 Hz, 2H), 7.05-7.13 (m, 1H), 7.31-7.36 (m, 2H), 7.43-7.51 (m, 2H); ESI (m/z) 390 (M+H)⁺.

### Intermediate 35

2-(2,6-Difluorophenyl)-2-oxoethyl 5-amino-3-methyl-1-(p yridin-2-yl)-1H-pyrazole-4-carboxylate

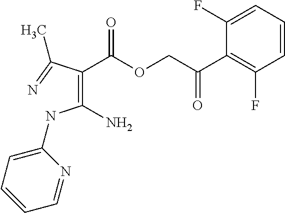 [SMILES: CC(=N)/C(C(=O)OCC(=O)c1c(F)cccc1F)=C(/N)Nc1ccccn1]

The titled intermediate was prepared by the reaction of 5-amino-3-methyl-1-(pyridin-2-yl)-1H-pyrazole-4-carboxylic acid with 2-bromo-1-(2,6-difluorophenyl)ethanone (1.1 g, 4.67 mmol) using potassium fluoride (340 mg, 5.83 mmol) in dry DMF (8.5 mL) as per the procedure described in Step 4 of Intermediate 1 to yield 1.08 g of the product as a solid. ¹H NMR (300 MHz, DMSO-d₆): δ 2.65 (s, 3H), 5.26 (s, 2H), 5.51 (br s, 2H), 7.24-7.29 (m, 3H), 7.67-7.83 (m, 4H).

### Intermediate 36

2-(2,6-Difluorophenyl)-2-oxoethyl 5-amino-1-(3,4-difluorophenyl)-3-methyl-1H-pyrazole-4-carboxylate

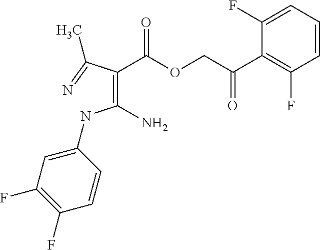 [SMILES: CC(=N)/C(C(=O)OCC(=O)c1c(F)cccc1F)=C(/N)Nc1ccc(F)c(F)c1]

The titled compound was prepared by the reaction of 5-amino-1-(3,4-difluorophenyl)-3-methyl-1H-pyrazole-4-carboxylic acid (800 mg, 3.16 mmol) with 2,6-difluorophenacylbromide (743 mg, 3.16 mmol) in the presence of potassium fluoride (275 mg, 4.73 mmol) in dry DMF (8.0 mL) at RT as per the procedure described in Step 4 of Intermediate 1 to yield 767 mg of the product as a solid. ¹H NMR (300 MHz, DMSO-d₆): δ 2.22 (s, 3H), 5.27 (s, 2H), 6.53 (s, 2H), 7.28 (t, J=7.8 Hz, 2H), 7.35-7.40 (m, 1H), 7.55-7.72 (m, 3H); ESI (m/z) 408 (M+H)⁺.

### Intermediate 37

2-(2-Fluoro-4-methoxyphenyl)-2-oxoethyl 5-amino-1-(4-fluorophenyl)-3-methyl-1H-pyrazole-4-carboxylate

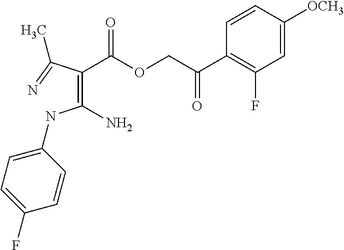 [SMILES: COc1ccc(C(=O)COC(=O)/C(C(C)=N)=C(\N)Nc2ccc(F)cc2)c(F)c1]

The titled compound was prepared by the reaction of 2-bromo-1-(2-fluoro-4-methoxyphenyl)ethanone (1.05 g, 4.25 mmol) with 5-amino-1-(4-fluorophenyl)-3-methyl-1H-pyrazole-4-carboxylic acid (1.0 g, 4.25 mmol) using potassium fluoride (370 mg, 6.37 mmol) in dry DMF (10 mL) as per the procedure described in Step 4 of Intermediate 1 to yield 866 mg of the product as a solid. ¹H NMR (300 MHz, DMSO-d₆): δ 2.28 (s, 3H), 3.88 (s, 3H), 5.36 (s, 2H), 6.40 (s, 2H), 6.95-7.05 (m, 2H), 7.36 (t, J=9.0 Hz, 2H), 7.54-7.59 (m, 2H), 7.88 (t, J=8.7 Hz, 1H); ESI (m/z) 402 (M+H)⁺.

### Intermediate 38

2-(2-Fluoro-4-methoxyphenyl)-2-oxoethyl 5-amino-1-(3,4-difluorophenyl)-3-methyl-1H-pyrazole-4-carboxylate

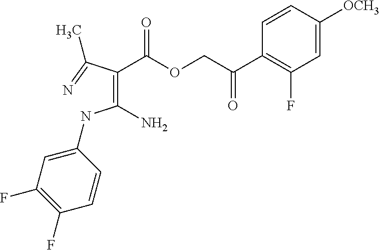 [SMILES: COc1ccc(C(=O)COC(=O)/C(C(C)=N)=C(\N)Nc2ccc(F)c(F)c2)c(F)c1]

The titled intermediate was prepared by the reaction of 2-bromo-1-(2-fluoro-4-methoxyphenyl)ethanone (927 mg, 3.75 mmol) with 5-amino-1-(3,4-difluorophenyl)-3-methyl-1H-pyrazole-4-carboxylic acid (950 mg, 3.75 mmol) using potassium fluoride (327 mg, 5.63 mmol) in dry DMF (10 mL) as per the procedure described in Step 4 of Intermediate 1 to yield 879 mg of the product as a solid. ¹H NMR (300 MHz, DMSO-d₆): δ 2.28 (s, 3H), 3.88 (s, 3H), 5.36 (s, 2H), 6.53 (s, 2H), 6.94-7.05 (m, 2H), 7.39-7.42 (m, 1H), 7.59-7.65 (m, 2H), 7.88 (t, J=8.4 Hz, 1H).

### Intermediate 39

2-(2-Chlorophenyl)-2-oxoethyl 5-amino-1-methyl-3-(trifluoromethyl)-1H-pyrazole-4-carboxylate

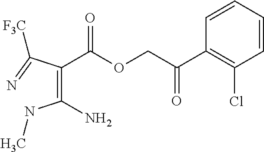 [SMILES: CN/C(N)=C(\C(=N)C(F)(F)F)C(=O)OCC(=O)c1ccccc1Cl]

### Step 1: Sodium-3-cyano-4-ethoxy-1,1,1-trifluoro-4-oxobut-2-en-2-olate.

To a stirred suspension of sodium metal (1.18 g, 51.72 mmol) in ethanol (19 mL), ethyl cyanoacetate (5.2 mL, 49.26 mmol) was added slowly at RT and the mixture was stirred for 1 h. The ethyl trifluoroacetate (7.0 g, 49.26 mmol) was added to the reaction mixture and stirred for 3 h at RT. The mixture was concentrated under reduced pressure and the residue was triturated with hexane (20 mL). The solvent was evaporated under vacuum to yield 11.3 g of the titled product as oil. ¹H NMR (300 MHz, CD₃COCD₃): δ 1.23 (t, J=6.9 Hz, 3H), 4.11 (q, J=6.9 Hz, 2H).

### Step 2: Ethyl-5-amino-1-methyl-3-(trifluoromethyl)-1H-pyrazole-4-carboxylate

To a stirred solution of Step 1 intermediate (11.0 g, 47.59 mmol) in dimethyl carbonate (90 mL) were added methyl hydrazine sulfate (13.72 g, 95.19 mmol), molecular sieves (12 g) and trifluoroacetic acid (3.64 mL, 47.59 mmol) at RT. The reaction mixture was refluxed overnight. The mixture was cooled to room temperature and filtered off the molecular sieves. The filtrate was concentrated under reduced pressure to afford 3.87 g of the titled product as a solid. ¹H NMR (300 MHz, CDCl₃): δ 1.32 (t, J=6.6 Hz, 3H), 3.64 (s, 3H), 4.27 (q, J=6.9 Hz, 2H), 5.19 (br s, 2H).

### Step 3: 5-Amino-1-methyl-3-(trifluoromethyl)-1H-pyrazole-4-carboxylic acid

To a stirred solution of Step 2 intermediate (3.8 g, 16.02 mmol) in ethanol (16 mL) was added an aqueous solution of potassium hydroxide (2.0 M, 16 mL, 24.03 mmol) and the mixture was refluxed overnight. The reaction mixture was cooled to RT, concentrated under reduced pressure and the residue was diluted with water (5.0 mL). The aqueous mixture was acidified with 1 N citric acid till pH 3-4. The solid precipitated was filtered and dried to afford 2.3 g of the desired product. ¹H NMR (300 MHz, DMSO-d₆): δ 3.60 (s, 3H), 6.53 (s, 2H), 12.40 (s, 1H).

### Step 4: 2-(2-Chlorophenyl)-2-oxoethyl 5-amino-1-methyl-3-(trifluoromethyl)-1H-pyrazole-4-carboxylate

To a stirred solution of Step 3 intermediate (900 mg, 4.30 mmol) in DMF (9.0 mL) were added 2-bromo-1-(2-chlorophenyl)ethanone (1.2 g, 5.16 mmol) and potassium fluoride (375 mg, 6.45 mmol) at RT. The mixture was stirred overnight at RT. The reaction mixture was quenched with water (20 mL). The precipitated solid was filtered and dried under vacuum. The crude compound was purified by silica gel column chromatography to yield 1.03 g of the titled intermediate as a solid. ¹H NMR (300 MHz, DMSO-d₆): δ 3.63 (s, 3H), 5.38 (s, 2H), 6.72 (s, 2H), 7.44-7.55 (m, 1H), 7.59 (d, J=3.9 Hz, 2H), 7.80 (d, J=7.8 Hz, 1H).

### Intermediate 40

2-(2-Fluorophenyl)-2-oxoethyl 5-amino-1-methyl-3-(trifluoromethyl)-1H-pyrazole-4 carboxylate

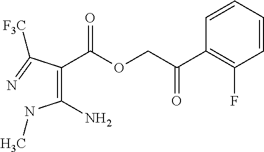 [SMILES: CN/C(N)=C(\C(=N)C(F)(F)F)C(=O)OCC(=O)c1ccccc1F]

The titled compound was prepared by the reaction of 5-amino-1-methyl-3-(trifluoromethyl)-1H-pyrazole-4-carboxylic acid (500 mg, 2.39 mmol) with 2-bromo-1-(2-fluorophenyl)ethanone (622 mg, 2.86 mmol) using potassium fluoride (208 mg, 4.30 mmol) in dry DMF (5.0 mL) as per the procedure described in Step 4 of Intermediate 1 to yield 642 mg of the product as a solid. ¹H NMR (300 MHz, DMSO-d₆): δ 3.64 (s, 3H), 5.40 (s, 2H), 6.72 (s, 2H), 7.35-7.47 (m, 2H), 7.67-7.75 (m, 1H), 7.90 (t, J=7.2 Hz, 1H).

### Intermediate 41

2-(4-Fluorophenyl)-2-oxoethyl 5-amino-1-methyl-3-(trifluoromethyl)-1H-pyrazole-4 carboxylate

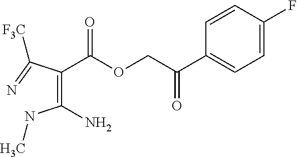 [SMILES: CN/C(N)=C(\C(=N)C(F)(F)F)C(=O)OCC(=O)c1ccc(F)cc1]

The titled compound was prepared by the reaction of 5-amino-1-methyl-3-(trifluoromethyl)-1H-pyrazole-4-carboxylic acid (500 mg, 2.39 mmol) with 2-bromo-1-(4-fluorophenyl)ethanone (622 mg, 2.86 mmol) using potassium fluoride (208 mg, 3.58 mmol) in dry DMF (5.0 mL) as per the procedure described in Step 4 of Intermediate 1 to yield 622 mg of the product as a solid. ¹H NMR (300 MHz, DMSO-d₆): δ 3.64 (s, 3H), 5.58 (s, 2H), 6.71 (s, 2H), 7.41 (t, J=8.7 Hz, 2H), 8.04-8.10 (m, 2H).

### Intermediate 42

2-(4-Chlorophenyl)-2-oxoethyl 5-amino-1-methyl-3-(trifluoromethyl)-1H-pyrazole-4 carboxylate

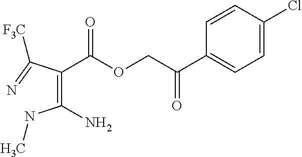 [SMILES: CN/C(N)=C(\C(=N)C(F)(F)F)C(=O)OCC(=O)c1ccc(Cl)cc1]

The titled compound was prepared by the reaction of 5-amino-1-methyl-3-(trifluoromethyl)-1H-pyrazole-4-carboxylic acid (500 mg, 2.39 mmol) with 2-bromo-1-(4-chlorophenyl)ethanone (669 mg, 2.86 mmol) using potassium fluoride (208 mg, 3.58 mmol) in dry DMF (5.0 mL) as per the procedure described in Step 4 of Intermediate 1 to yield 760 mg of the product as a solid. ¹H NMR (300 MHz, DMSO-d₆): δ 3.63 (s, 3H), 5.57 (s, 2H), 6.71 (s, 2H), 7.64 (d, J=8.4 Hz, 2H), 8.00 (d, J=8.7 Hz, 2H).

### Intermediate 43

2-(2-Chloro-4-fluorophenyl)-2-oxoethyl 5-amino-1-methyl-3-(trifluoromethyl)-1H-pyrazole-4-carboxylate

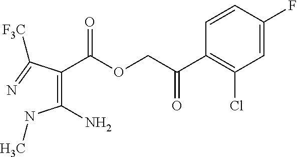 [SMILES: CN/C(N)=C(\C(=N)C(F)(F)F)C(=O)OCC(=O)c1ccc(F)cc1Cl]

The titled compound was prepared by the reaction of 5-amino-1-methyl-3-(trifluoromethyl)-1H-pyrazole-4-carboxylic acid (600 mg, 2.86 mmol) with 2-bromo-1-(2-chloro-4-fluorophenyl)ethanone (866 mg, 3.44 mmol) using potassium fluoride (250 mg, 4.30 mmol) in dry DMF (6.0 mL) as per the procedure described in Step 4 of Intermediate 1 to yield 722 mg of the product as a solid. ¹H NMR (300 MHz, DMSO-d₆): δ 3.63 (s, 3H), 5.38 (s, 2H), 6.72 (s, 2H), 7.40 (t, J=6.3 Hz, 1H), 7.62 (d, J=6.6 Hz, 1H), 7.93 (t, J=6.6 Hz, 1H).

### Intermediate 44

2-(2-Chloro-6-fluorophenyl)-2-oxoethyl 5-amino-1-methyl-3-(trifluoromethyl)-1H-pyrazole-4-carboxylate

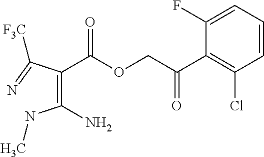 [SMILES: CN/C(N)=C(\C(=N)C(F)(F)F)C(=O)OCC(=O)c1c(F)cccc1Cl]

The titled compound was prepared by the reaction of 5-amino-1-methyl-3-(trifluoromethyl)-1H-pyrazole-4-carboxylic acid (700 mg, 3.34 mmol) with 2-bromo-1-(2-chloro-6-fluorophenyl)ethanone (1.0 g, 4.01 mmol) using potassium fluoride (291 mg, 5.02 mmol) in dry DMF (7.0 mL) as per the procedure described in Step 4 of Intermediate 1 to yield 567 mg of the product as a solid. ¹H NMR (300 MHz, DMSO-d₆): δ 3.62 (s, 3H), 5.26 (s, 2H), 6.73 (s, 2H), 7.32-7.50 (m, 1H), 7.55-7.65 (m, 2H).

### Intermediate 45

2-(3-Chloropyridin-4-yl)-2-oxoethyl 5-amino-1-methyl-3-(trifluoromethyl)-1H-pyrazole-4-carboxylate

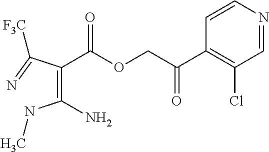 [SMILES: CN/C(N)=C(\C(=N)C(F)(F)F)C(=O)OCC(=O)c1ccncc1Cl]

The titled compound was prepared by the reaction of 5-amino-1-methyl-3-(trifluoromethyl)-1H-pyrazole-4-carboxylic acid (1.0 g, 4.78 mmol) with 2-bromo-1-(3-chloropyridin-4-yl)ethanone (2.12 g, 4.78 mmol) using triethylamine (2.0 mL, 14.34 mmol) in acetonitrile (24 mL) as per the procedure described in Step 4 of Intermediate 1 to yield 1.06 g of the product as a solid. ¹H NMR (300 MHz, DMSO-d₆): δ 3.62 (s, 3H), 5.40 (s, 2H), 6.73 (s, 2H), 7.75-7.78 (m, 1H), 8.70-8.72 (m, 1H), 8.81 (s, 1H).

### Intermediate 46

2-(2-Fluoro-4-methoxyphenyl)-2-oxoethyl 5-amino-1-methyl-3-(trifluoromethyl)-1H-pyrazole-4-carboxylate

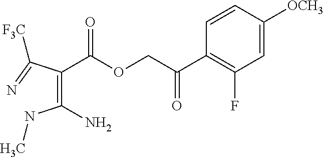 [SMILES: CN/C(N)=C(\C(=N)C(F)(F)F)C(=O)OCC(=O)c1ccc(OC)cc1F]

The titled compound was prepared by the reaction of 5-amino-1-methyl-3-(trifluoromethyl)-1H-pyrazole-4-carboxylic acid (950 mg, 4.54 mmol) with 2-bromo-1-(2-fluoro-4-methoxyphenyl)ethanone (1.34 g, 5.45 mmol) using potassium fluoride (396 mg, 6.81 mmol) in dry DMF (10.0 mL) as per the procedure described in Step 4 of Intermediate 1 to yield 1.01 g of the product as a solid. ¹H NMR (300 MHz, DMSO-d₆): δ 3.64 (s, 3H), 3.87 (s, 3H), 5.41 (s, 2H), 6.72 (br s, 2H), 6.94-7.04 (m, 2H), 7.88 (t, J=8.7 Hz, 1H); ESI (m/z) 377 (M+H)⁺.

### Intermediate 47

2-(2-Chloro-4-methoxyphenyl)-2-oxoethyl 5-amino-1-methyl-3-(trifluoromethyl)-1H-pyrazole-4-carboxylate

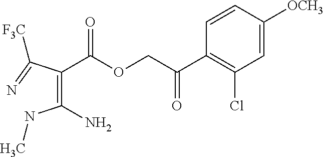 [SMILES: CN/C(N)=C(\C(=N)C(F)(F)F)C(=O)OCC(=O)c1ccc(OC)cc1Cl]

The titled compound was prepared by the reaction of 5-amino-1-methyl-3-(trifluoromethyl)-1H-pyrazole-4-carboxylic acid (900 mg, 4.30 mmol) with 2-bromo-1-(2-chloro-4-methoxyphenyl)ethanone (1.13 g, 4.29 mmol) using potassium fluoride (375 mg, 6.44 mmol) in dry DMF (9.0 mL) as per the procedure described in Step 4 of Intermediate 1 to yield 256 mg of the product as a solid. ¹H NMR (300 MHz, DMSO-d₆): δ 3.63 (s, 3H), 3.82 (s, 3H), 5.40 (s, 2H), 6.71 (s, 2H), 7.04-7.07 (m, 1H), 7.16 (s, 1H), 7.89 (d, J=8.4 Hz, 1H); APCI (m/z) 392 (M+H)⁺.

### Intermediate 48

2-(2-Chloro-5-methoxyphenyl)-2-oxoethyl 5-amino-1-methyl-3-(trifluoromethyl)-1H-pyrazole-4-carboxylate

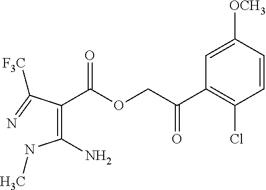 [SMILES: CN/C(N)=C(\C(=N)C(F)(F)F)C(=O)OCC(=O)c1cc(OC)ccc1Cl]

The titled intermediate was prepared by the reaction of 5-amino-1-methyl-3-(trifluoromethyl)-1H-pyrazole-4-carboxylic acid (1.0 g, 4.78 mmol) with 2-bromo-1-(2-chloro-5-methoxyphenyl)ethanone (1.8 g, 4.78 mmol) with using potassium fluoride (416 mg, 7.17 mmol) in dry DMF (10 mL) as per the procedure described in Step 4 of Intermediate 1 to yield 1.2 g of the product as a solid. ¹H NMR (300 MHz, DMSO-d₆): δ 3.63 (s, 3H), 3.81 (s, 3H), 5.39 (s, 2H), 6.72 (br s, 2H), 7.14-7.18 (m, 1H), 7.30-7.32 (m, 1H), 7.48 (d, J=8.7 Hz, 1H).

### Intermediate 49

2-(2,5-Dichlorophenyl)-2-oxoethyl 5-amino-1-methyl-3-(trifluoromethyl)-1H-pyrazole-4-carboxylate.

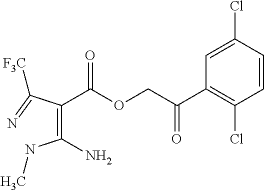 [SMILES: CN/C(N)=C(\C(=N)C(F)(F)F)C(=O)OCC(=O)c1cc(Cl)ccc1Cl]

The titled compound was prepared by the reaction of 2-bromo-1-(2,5-dichlorophenyl)ethanone (1.27 g, 4.76 mmol) with 5-amino-1-methyl-3-(trifluoromethyl)-1H-pyrazole-4-carboxylic acid (1.0 g, 4.78 mmol) using potassium fluoride (416 mg, 7.16 mmol) in dry DMF (10.0 mL) as per the procedure described in Step 4 of Intermediate 1 to yield 1.12 g of the product as a solid. ¹H NMR (300 MHz, DMSO-d₆): δ 3.63 (s, 3H), 5.38 (s, 2H), 6.73 (s, 2H), 7.60-7.67 (m, 2H), 7.89 (s, 1H).

### Intermediate 50

2-(2,4-Dimethoxyphenyl)-2-oxoethyl 5-amino-1-methyl-3-(trifluoromethyl)-1H-pyrazole-4-carboxylate.

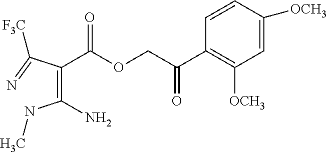 [SMILES: CN/C(N)=C(\C(=N)C(F)(F)F)C(=O)OCC(=O)c1ccc(OC)cc1OC]

The titled compound was prepared by the reaction of 5-amino-1-methyl-3-(trifluoromethyl)-1H-pyrazole-4-carboxylic acid (750 mg, 3.58 mmol) with 2-bromo-1-(2,4-dimethoxyphenyl)ethanone (1.11 g, 4.30 mmol) using triethylamine (545 mg, 5.38 mmol) in acetonitrile (10 mL) as per the procedure described in Step 4 of Intermediate 1 to yield 940 mg of the product as a solid. ¹H NMR (300 MHz, DMSO-d₆): δ 3.63 (s, 3H), 3.87 (s, 3H), 3.96 (s, 3H), 5.28 (s, 2H), 6.68-6.71 (m, 4H), 7.80 (d, J=9.0 Hz, 1H); ESI (m/z) 388 (M)⁺.

### Intermediate 51

2-(4-Chloro-2-fluorophenyl)-2-oxoethyl 5-amino-1-methyl-3-(trifluoromethyl)-1H-pyrazole-4-carboxylate

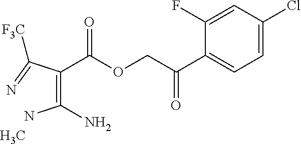

The titled intermediate was prepared by the reaction of (5-amino-1-methyl-3-(trifluoromethyl)-1H-pyrazole-4-carboxylic acid (1.0 g, 4.78 mmol) with 2-bromo-1-(4-chloro-2-fluorophenyl)ethanone (1.2 g, 4.78 mmol) using potassium fluoride (416 mg, 7.17 mmol) in dry DMF (10 mL) as per the procedure described in Step 4 of Intermediate 1 to yield 1.3 g of the product as a solid. ¹H NMR (300 MHz, DMSO-d₆): δ 3.63 (s, 3H), 5.39 (s, 2H), 6.72 (s, 2H), 7.50 (d, J=8.4 Hz, 1H), 7.72 (d, J=9.0 Hz, 1H), 7.92 (t, J=8.4 Hz, 1H); ESI (m/z) 402 (M+H)⁺.

### Intermediate 52

2-(4-Methoxyphenyl)-2-oxoethyl 5-amino-1-methyl-3-(trifluoromethyl)-1H-pyrazole-4-carboxylate

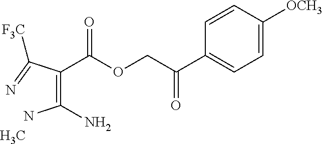

The titled intermediate was prepared by the reaction of 5-amino-1-methyl-3-(trifluoromethyl)-1H-pyrazole-4-carboxylic acid (2.0 g, 9.56 mmol) with 2-Bromo-1-(4-methoxyphenyl)ethanone (2.2 g, 9.56 mmol) using potassium fluoride (833 mg, 14.34 mmol) in dry DMF (20 mL) as per the procedure described in Step 4 of Intermediate 1 to yield 2.31 g of the product as a solid. ¹H NMR (300 MHz, DMSO-d₆): δ 3.64 (s, 3H), 3.86 (s, 3H), 5.53 (s, 2H), 6.72 (s, 2H), 7.09 (d, J=9.0 Hz, 2H), 7.97 (d, J=9.0 Hz, 2H).

### Intermediate 53

2-[4-(1H-Imidazol-1-yl)phenyl]-2-oxoethyl 5-amino-1-methyl-3-(trifluoromethyl)-1H-pyrazole-4-carboxylate

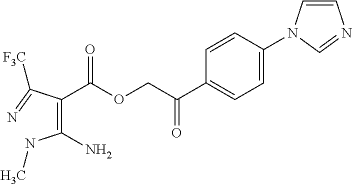 [SMILES: CN/C(N)=C(\C(=N)C(F)(F)F)C(=O)OCC(=O)c1ccc(-n2ccnc2)cc1]

The titled compound was prepared by the reaction of 5-amino-1-methyl-3-(trifluoromethyl)-1H-pyrazole-4-carboxylic acid (1 g, 4.78 mmol) with 2-bromo-1-[4-(1H-imidazol-1-yl)phenyl]ethanone hydrobromide (1.65 g, 4.78 mmol) using triethylamine (5.33 mL, 38.24 mmol) in acetonitrile (24 mL) as per the procedure described in Step 4 of Intermediate 1 to yield 286 mg of the product as a solid. ¹H NMR (300 MHz, DMSO-d₆): δ 3.64 (s, 3H), 5.62 (s, 2H), 6.73 (s, 2H), 7.16 (s, 1H), 7.88-7.93 (m, 3H), 8.13 (d, J=8.7 Hz, 2H), 8.47 (s, 1H).

### Intermediate 54

2-Oxo-2-(pyridine-4-yl)ethyl 5-amino-1-methyl-3-(trifluoromethyl)-1H-pyrazole-4-carboxylate.

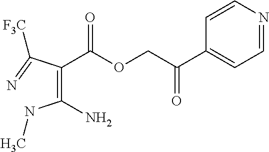 [SMILES: CN/C(N)=C(\C(=N)C(F)(F)F)C(=O)OCC(=O)c1ccncc1]

The titled compound was prepared by the reaction of 5-amino-1-methyl-3-(trifluoromethyl)-1H-pyrazole-4-carboxylic acid (600 mg, 2.86 mmol) with 2-bromo-1-(pyridin-4-yl)ethanone (803 mg, 2.86 mmol) using triethylamine (3.2 mL, 22.95 mmol) in acetonitrile (15 mL) as per the procedure described in Step 4 of Intermediate 1 to yield 358 mg of the product as a solid.

¹H NMR (300 MHz, DMSO-d₆): δ 3.63 (s, 3H), 5.61 (s, 2H), 6.73 (s, 2H), 7.85 (d, J=5.7 Hz, 2H), 8.85 (d, J=6.0 Hz, 2H).

### Intermediate 55

2-(2-Chlorophenyl)-2-oxoethyl 5-amino-3-ethyl-1-methyl-1H-pyrazole-4-carboxylate

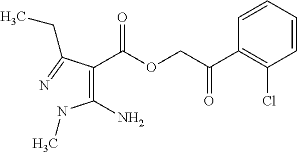 [SMILES: CCC(=N)/C(C(=O)OCC(=O)c1ccccc1Cl)=C(/N)NC]

### Step 1: Ethyl 2-cyano-3-ethoxypent-2-enoate

To a stirred solution of ethyl cyanoacetate (6.0 g, 53.04 mmol) in acetic anhydride (60 mL), triethyl orthopropionate (11.73 mL, 58.34 mmol) was added at RT and the reaction mixture was heated to 140° C. overnight. The mixture was concentrated under reduced pressure. The residue thus obtained was purified by silica gel column chromatography to yield 5.1 g of the titled product as viscous liquid. ¹H NMR (300 MHz, CDCl₃): δ 1.19 (t, J=7.8 Hz, 2H), 1.30 (t, J=7.2 Hz, 2H), 1.42 (t, J=7.2 Hz, 2H), 2.99 (q, J=7.8 Hz, 2H), 4.21 (q, J=7.2 Hz, 2H), 4.31 (q, J=7.8 Hz, 2H).

### Step 2: Ethyl 5-amino-3-ethyl-1-methyl-1H-pyrazole-4-carboxylate

The titled compound was prepared by the reaction of Step 1 intermediate (2.7 g, 13.68 mmol) with methyl hydrazine (742 μL, 13.68 mmol) in dry ethanol (27 mL) as per the procedure described in Step 2 of Intermediate 1 to yield 1.71 g of the product as oil. ¹H NMR (300 MHz, CDCl₃): δ 1.21 (t, J=7.8 Hz, 2H), 1.35 (t, J=7.2 Hz, 2H), 2.75 (q, J=7.8 Hz, 2H), 3.58 (s, 3H), 4.28 (q, J=7.8 Hz, 2H), 5.04 (br s, 2H).

### Step 3: 5-Amino-3-ethyl-1-methyl-1H-pyrazole-4-carboxylic acid

The titled compound was prepared by the ester hydrolysis of Step 2 intermediate (1.7 g, 8.61 mmol) using aqueous solution of potassium hydroxide (2.0 M, 6 mL, 17.23 mmol) in ethanol (17 mL) as per the procedure described in Step 3 of Intermediate 1 to yield 1.51 g of the product as oil. ¹H NMR (300 MHz, CDCl₃): δ 1.18 (t, J=7.8 Hz, 2H), 2.51 (q, J=7.8 Hz, 2H), 3.60 (s, 3H), 5.86 (br s, 1H), 12.40 (s, 1H).

### Step 4: 2-(2-Chlorophenyl)-2-oxoethyl 5-amino-3-ethyl-1-methyl-1H-pyrazole-4-carboxylate

The titled compound was prepared by the reaction of Step 3 intermediate (1.5 g, 4.30 mmol) with 2-bromo-1-(2-chlorophenyl)ethanone (2.48 g, 10.61 mmol) using potassium fluoride (780 mg, 13.30 mmol) in dry DMF (15.0 mL) as per the procedure described in Step 4 of Intermediate 1 to yield 650 mg of the product as a solid. ¹H NMR (300 MHz, DMSO-d₆): δ 1.24 (t, J=9.0 Hz, 3H), 2.70 (q, J=9.0 Hz, 2H), 3.55 (s, 3H), 5.09 (s, 2H), 5.33 (s, 2H), 7.30-7.50 (m, 2H), 7.64 (d, J=7.8 Hz, 2H).

### Intermediate 56

2-(2-chlorophenyl)-2-oxoethyl 3-amino-5-(2-fluorobenzyl)-1-methyl-1H-pyrazole-4-carboxylate.

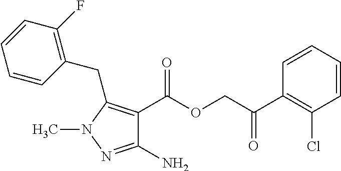 [SMILES: Cn1nc(N)c(C(=O)OCC(=O)c2ccccc2Cl)c1Cc1ccccc1F]

### Step 1: Ethyl (2Z)-2-cyano-4-(2-fluorophenyl)-3-hydroxybut-2-enoate

To a stirred solution of (2-fluorophenyl)acetic acid (5.0 g, 32.44 mmol) in dichloromethane (50 mL), oxalyl chloride (4.2 mL, 48.61 mmol) was added at 0° C. and the reaction mixture stirred for 3 h at room temperature. The reaction was concentrated under reduced pressure to obtain (2-fluorophenyl)acetyl chloride (5.6 g, 32.45 mmol). The ethyl cyanoacetate (3.46 mL, 32.53 mmol) was added to stirred suspension of sodium hydride (60% w/w, 2.6 g, 64.88 mmol) in THF (15 mL) and the mixture was stirred at room temperature for 1 h. The mixture was cooled to 0° C. and (2-fluorophenyl)acetyl chloride (5.6 g, 32.45 mmol) was added to the reaction mixture. The mixture was stirred at room temperature overnight. The reaction mixture was quenched with 2N sulfuric acid till pH 2-3. The aqueous mixture was extracted with ethyl acetate (2×300 mL) and the combined organic layers were dried over sodium sulfate. The solution was concentrated under reduced pressure and purified by silica gel column chromatography to yield 7.02 g of the product as a solid. ¹H NMR (300 MHz, CDCl₃): δ 1.37 (t, J=6.9 Hz, 3H), 3.98 (s, 2H), 4.36 (q, J=6.9 Hz, 2H), 7.09-7.16 (m, 2H), 7.26-7.33 (m, 2H), 13.74 (s, 1H).

### Step 2: Ethyl 3-chloro-2-cyano-4-[2-(fluoromethyl)phenyl]but-2-enoate

To a stirred solution of ethyl 2-cyano-4-(2-fluorophenyl)-3-hydroxybut-2-enoate (5.7 g, 22.88 mmol) in dichloromethane (57 mL), phosphorous oxychloride (2.3 mL, 25.16 mmol) and triethylamine 4.78 mL, 34.32 mmol) were added at room temperature. The reaction mixture was heated to 50° C. and stirred for 18 h. The reaction mixture was cooled to RT and quenched with saturated sodium bicarbonate solution (200 mL). The layer was separated and the aqueous layer was extracted with dichloromethane (3×250 mL). The organic layer was dried over anhydrous sodium sulfate, filtered and concentrated under reduced pressure. The residue thus obtained was purified by silica gel column chromatography to yield 1.96 g of the titled product as viscous liquid. ¹H NMR (300 MHz, CDCl₃): δ 1.39 (t, J=6.9 Hz, 3H), 4.38 (q, J=6.9 Hz, 2H), 4.56 (s, 2H), 7.08-7.15 (m, 2H), 7.21-7.32 (m, 2H).

### Step 3: Ethyl 3-amino-5-(2-fluorobenzyl)-1-methyl-1H-pyrazole-4-carboxylate

The titled compound was prepared by the reaction of Step 1 intermediate (1.0 g, 3.73 mmol) with aqueous methyl hydrazine (86%, 800 μL, 3.73 mmol) in 10% aqueous sodium hydroxide (10 mL) as per the procedure described in Step 2 of Intermediate 1 to yield 1.2 g of the product as oil. The compound was carried forward to the next step without purification or characterization.

### Step 4: 3-Amino-5-(2-flu orobenzyl)-1-methyl-1H-pyrazole-4-carboxylic acid

The titled compound was prepared by the ester hydrolysis of Step 3 intermediate (1.18 g, 4.25 mmol) using aqueous potassium hydroxide (4.0 mL, 6.38 mmol) in ethanol (4.0 mL) as per the procedure described in Step 3 of Intermediate 1 to yield 730 mg of the product as a solid. ¹H NMR (300 MHz, DMSO-d₆): δ 3.60 (s, 3H), 4.27 (s, 2H), 6.90 (t, J=7.2 Hz, 1H), 7.08-7.26 (m, 5H).

### Step 5: 2-(2-chlorophenyl)-2-oxoethyl 3-amino-5-(2-fluorobenzyl)-1-methyl-1H-pyrazole-4-carboxylate

The titled compound was prepared by the reaction of Step 4 intermediate (730 mg, 2.92 mmol) with 2-bromo-1-(2-chlorophenyl)ethanone (683 mg, 2.92 mmol) using potassium fluoride (255 mg, 4.39 mmol) in dry DMF (7.0 mL) as per the procedure described in Step 4 of Intermediate 1 to yield 446 mg of the product as a solid. ¹H NMR (300 MHz, CDCl₃): δ 3.63 (s, 3H), 4.32 (s, 2H), 5.37 (s, 2H), 7.04-7.23 (m, 3H), 7.22-7.25 (m, 1H), 7.33-7.42 (m, 3H), 7.63 (d, J=7.2 Hz, 1H).

### Intermediate 57

2-(2-Chlorophenyl)-2-oxoethyl 3-amino-5-(4-fluorophenyl)-1-methyl-1H-pyrazole-4-carboxylate

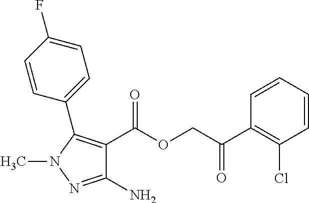 [SMILES: Cn1nc(N)c(C(=O)OCC(=O)c2ccccc2Cl)c1-c1ccc(F)cc1]

### Step 1: Ethyl 2-cyano-3-(4-fluorophenyl)-3-hydroxyprop-2-enoate

To a stirred solution of 4-fluorobenzoic acid (20 g, 142.73 mmol) in DMF (5.0 mL) and dichloromethane (200 mL), oxalyl chloride (25 mL, 285.46 mmol) was added at 0° C. and the reaction mixture was stirred at RT for 5 h. The solvent was evaporated under reduced pressure. The residue was diluted with toluene (300 mL). Ethyl cyanoacetate (8.07 g, 71.30 mmol) and triethylamine (20 mL, 142.73 mmol) were added to the mixture at room temperature. The mixture was stirred overnight at at RT. The reaction mixture was diluted with water (300 mL) and extracted with ethyl acetate (2×300 mL). The organic layer was dried over anhydrous sodium sulfate and concentrated under reduced pressure to yield 17.25 g of the titled product as oil. The intermediate was directly used in the next step.

### Step 2: Ethyl 3-chloro-2-cyano-3-(4-fluorophenyl)prop-2-enoate

To a stirred solution of Step 1 intermediate (17.22 g, 73.21 mmol) in dry dichloromethane (173 mL), phosphoryl oxychloride (7.4 mL, 80.53 mmol) and triethylamine (15.5 mL, 109.81 mmol) were added at RT. The reaction mixture was heated to 45° C. and stirred at the same temperature overnight. The mixture was cooled to room temperature and acidified with 5.0 N HCl (100 mL). The mixture was extracted with dichlomethane (2×200 mL). The organic layer was washed with sodium bicarbonate (200 mL) and dried over anhydrous sodium sulfate. The complete evaporation of solvent gave 7.0 g of the titled product as oil. The intermediate was as such used in the next step.

### Step 3: Ethyl 3-amino-5-(4-fluorophenyl)-1-methyl-1H-pyrazole-4-carboxylate

To a stirred solution of Step 2 intermediate (6.7 g, 26.41 mmol) in 1.0 N aqueous sodium hydroxide (67 mL) was added methyl hydrazine sulfate (3.8 g, 26.41 mmol) and the reaction mixture was stirred at room temperature for 16 h. The mixture was diluted with water and extracted with ethyl acetate (3×150 mL). The organic layer was dried over anhydrous sodium sulfate. The solution was concentrated under reduced pressure and the obtained product was purified by flash silica gel column chromatography to afford 880 mg of the titled product as a solid. ¹H NMR (300 MHz, DMSO-d₆): δ 0.97 (t, J=6.9 Hz, 3H), 3.42 (s, 3H), 3.96 (q, J=6.9 Hz, 2H), 5.45 (s, 2H), 7.30 (t, J=8.7 Hz, 2H), 7.45 (t, J=8.4 Hz, 2H).

### Step 4: 3-Amino-5-(4-fluorophenyl)-1-methyl-1H-pyrazole-4-carboxylic acid

The titled compound was prepared by the ester hydrolysis of Step 3 intermediate (870 mg, 3.30 mmol) using aqueous solution of potassium hydroxide (2.0 M, 3.3 mL, 4.95 mmol) in ethanol (3.3 mL) as per the procedure described in Step 3 of Intermediate 1 to yield 699 mg of the product as a solid. ¹H NMR (300 MHz, DMSO-d₆): δ 3.62 (s, 3H), 5.41 (s, 2H), 7.44 (t, J=8.7 Hz, 2H), 7.66 (t, J=8.4 Hz, 2H), 11.42 (br s, 1H).

### Step 5: 2-(2-Chlorophenyl)-2-oxoethyl 3-amino-5-(4-fluorophenyl)-1-methyl-1H-pyrazole-4-carboxylate

The titled compound was prepared by the reaction of Step 4 intermediate (690 mg, 2.933 mmol) with 2-bromo-1-(2-chlorophenyl)ethanone (821 mg, 3.52 mmol) using potassium fluoride (255 mg, 4.40 mmol) in dry DMF (7 mL) as per the procedure described in Step 4 of Intermediate 1 to yield 560 mg of the product as a solid. ¹H NMR (300 MHz, DMSO-d₆): δ 3.44 (s, 3H), 5.17 (s, 2H), 5.55 (s, 2H), 7.29 (t, J=8.7 Hz, 2H), 7.39-7.50 (m, 3H), 7.56 (d, J=3.9 Hz, 2H), 7.68 (d, J=7.2 Hz, 1H).

### Intermediate 58

2-(2-Chlorophenyl)-2-oxoethyl 3-amino-1,5-dimethyl-1H-pyrazole-4-carboxylate

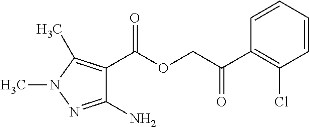 [SMILES: Cc1c(C(=O)OCC(=O)c2ccccc2Cl)c(N)nn1C]

### Step 1: 1-Benzylidene-2-methylhydrazine

To a stirred solution of benzaldehyde (4.0 g, 37.68 mmol) in dry ethanol (6.3 mL), methyl hydrazine (2.0 mL, 37.68 mmol) was added at room temperature and the mixture was refluxed for 1 h. The reaction mixture was concentrated under reduced pressure and the residue was diluted with water (300 mL). The aqueous mixture was extracted with ethyl acetate (2×200 mL). The organic layer was dried over anhydrous sodium sulfate and concentrated under reduced pressure to yield 3.5 g of the titled product as colorless oil. ¹H NMR (300 MHz, CDCl₃): δ 2.98 (s, 3H), 4.98 (s, 1 H,) 7.20-7.38 (m, 3H), 7.51-7.60 (m, 3H).

### Step 2: Ethyl 2-cyano-3-ethoxybut-2-enoate

A stirred mixture of ethyl cyanoacetate (10.0 g, 88.40 mmol) and triethyl orthoacetate (17.90 mL, 96.77 mmol) in acetic anhydride (100 mL) was refluxed for 24 h. The excess of acetic anhydride was removed under vacuum and the residue was purified by silica gel column chromatography to yield 2.7 g of the titled product as oil. ¹H NMR (300 MHz, CDCl₃): δ 1.30 (t, J=7.5 Hz, 3H), 1.42 (t, J=6.9 Hz, 3H), 2.60 (s, 3H), 4.15-4.35 (m, 4H).

### Step 3: Ethyl 3-amino-1,5-dimethyl-1H-pyrazole-4-carboxylate

To a stirred solution of Step 2 intermediate (2.7 g, 12.85 mmol) in dry toluene (11 mL), Step 1 intermediate (1.5 g, 11.17 mmol) was added at RT and the reaction mixture was refluxed overnight. The reaction mixture was cooled to room temperature and concentrated under reduced pressure. The residue was refluxed in a mixture of ethanol (15 mL) and concentrated hydrochloric acid (1.5 mL) for 1 h. The reaction mixture was cooled to room temperature and concentrated under reduced pressure. The residue was treated with 1.0 N HCl (50 mL) and extracted with chloroform (3×75 mL). The organic layer was dried over anhydrous sodium sulfate and concentrated under reduced pressure to yield 730 mg of the titled product as a solid. ¹H NMR (300 MHz, DMSO-d₆): δ 1.25 (d, J=6.6 Hz, 3H), 2.35 (s, 3H), 3.51 (s, 3H), 4.16 (q, J=6.9 Hz, 2H), 5.22 (s, 2H).

### Step 4: 3-Amino-1,5-dimethyl-1H-pyrazole-4-carboxylic acid

The titled compound was prepared by the ester hydrolysis of Step 3 intermediate (720 mg, 3.93 mmol) using aqueous solution of potassium hydroxide (2.0 M, 3.9 mL, 5.89 mmol) in ethanol (4.0 mL) as per the procedure described in Step 3 of Intermediate 1 to yield 515 mg of the product as a solid. ¹H NMR (300 MHz, DMSO-d₆): δ 2.34 (s, 3H), 3.50 (s, 3H), 5.42 (s, 2H), 11.38 (br s, 1H).

### Step 5: 2-(2-Chlorophenyl)-2-oxoethyl-3-amino-1,5-dimethyl-1H-pyrazole-4-carboxylate

The titled compound was prepared by the reaction of Step 4 intermediate (500 mg, 3.22 mmol) with 2-bromo-1-(2-chlorophenyl)ethanone (902 mg, 3.86 mmol) using potassium fluoride (280 mg, 4.81 mmol) in dry DMF (5.0 mL) as per the procedure described in Step 4 of Intermediate 1 to yield 260 mg of the product as a solid. ¹H NMR (300 MHz, DMSO-d₆): δ 2.33 (s, 3H), 3.53 (s, 3H), 5.34 (br s, 4H), 7.41-7.50 (m, 1H), 7.58 (d, J=3.9 Hz, 2H), 7.79 (d, J=7.8 Hz, 1H).

### Intermediate 59

3-(2-Chlorophenyl)-1-(4-hydroxy-1,5-dimethyl-1H-pyrazol-3-yl)prop-2-en-1-one

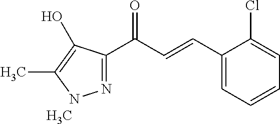

### Step 1: 1-(4-Hydroxy-1,5-dimethyl-1H-pyrazol-3-yl)ethanone

Methyl hydrazine sulfate (2.0 g, 13.87 mmol) was added to a stirred mixture of pyruvic aldehyde (40% in water, 14.9 mL, 83.24 mmol), acetic acid (1.2 mL, 20.80 mmol) and water (22 mL). The reaction mixture was refluxed for 3 h. The mixture was cooled to RT and diluted with water (100 mL). The layer was separated and the aqueous solution was extracted with ethyl acetate (2×250 mL). The combined organic layer was dried over anhydrous sodium sulfate and concentrated under reduced pressure to afford 1.70 g of the titled product as a solid. ¹H NMR (300 MHz, CDCl₃): δ 2.19 (s, 3H), 2.51 (s, 3H), 3.79 (s, 3H), 7.83 (br s, 1H).

### Step 2: 3-(2-Chlorophenyl)-1-(4-hydroxy-1,5-dimethyl-1H-pyrazol-3-yl)prop-2-en-1-one

To a stirred solution of 2-chlorobenzadehyde (380 μL, 3.24 mmol) in dry ethanol (5.0 mL), were added the Step 1 intermediate (500 mg, 3.24 mmol) and a solution of sodium hydroxide (557 mg, 13.93 mmol) in ethanol (5.0 mL). The reaction mixture was stirred at RT for 18 h. The solvents were recovered under reduced pressure and the residue was diluted with water (100 mL). The aqueous solution was extracted with ethyl acetate (2×100 mL). The organic layer was dried over anhydrous sodium sulfate and concentrated to afford 455 mg of the titled product as a solid. ¹H NMR (300 MHz, CDCl₃): δ 2.23 (s, 3H), 3.83 (s, 3H), 7.29-7.32 (m, 2H), 7.41-7.44 (m, 1H), 7.57-7.62 (m, 1H), 7.82-7.85 (m, 1H), 8.19 (s, 1H), 8.32-8.38 (m, 1H).

### Intermediate 60

2-(2-Chlorophenyl)-2-oxoethyl 2-amino-5-methylthiophene-3-carboxylate

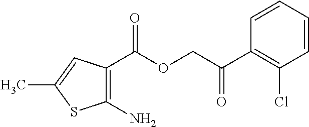 [SMILES: Cc1cc(C(=O)OCC(=O)c2ccccc2Cl)c(N)s1]

Step 1: Ethyl 2-amino-5-methylthiophene-3-carboxylate

To a stirred mixture of ethyl cyanoacetate (10.0 g, 88.40 mmol), sulfur (2.83 g, 88.40 mmol) and triethylamine (12.5 mL) in dry DMF (18 mL) was slowly added a solution of propionaldehyde (7.0 mL, 97.10 mmol) in ethanol (3.2 mL) at room temperature. The reaction mixture was heated to 60° C. for 1 h. The mixture was cooled to room temperature and quenched with water (100 mL). The aqueous mixture was extracted with ethyl acetate (2×250 mL). The organic layer was dried over anhydrous sodium sulfate. The solution was concentrated under reduced pressure to afford 12 g of the titled product as a solid. ¹H NMR (300 MHz, CDCl₃): δ 1.33 (t, J=6.6 Hz, 3H), 2.26 (s, 3H), 4.25 (q, J=6.9 Hz, 2H), 4.61 (br s, 2H), 6.62 (s, 1H).

### Step 2: 2-Amino-5-methylthiophene-3-carboxylic acid

To a stirred solution of Step 1 intermediate (2.5 g, 13.50 mmol) in THF (25 mL), water (8.0 mL) and methanol (16.5 mL) was added aqueous solution of lithium hydroxide [prepared by dissolving lithium hydroxide (2.83 g, 67.54 mmol) in water (42.5 mL)] at room temperature. The reaction mixture was stirred at 85° C. for 3 h. The mixture was concentrated under reduced pressure to remove the organic solvent. The residue was diluted with ethyl acetate (100 mL). The layer was separated and the aqueous layer was acidified with 1.0 N HCl till pH 4.0. The solid precipitated was filtered and dried to afford 1.4 g of the titled product. ¹H NMR (300 MHz, CDCl₃): δ 2.16 (s, 3H), 6.45 (s, 1H), 7.02 (br s, 2H), 11.74 (s, 1H).

### Step 3: 2-(2-Chlorophenyl)-2-oxoethyl-2-amino-5-methylthiophene-3-carboxylate

The titled compound was prepared by the reaction of Step 2 intermediate (2.0 g, 12.72 mmol) with 2-bromo-1-(2-chlorophenyl)ethanone (3.56 g, 15.26 mmol) using potassium fluoride (1.1 g, 19.08 mmol) in dry DMF (20 mL) as per the procedure described in Step 4 of Intermediate 1 to yield 3.05 g of the product as a solid. ¹H NMR (300 MHz, DMSO-d₆): δ 2.26 (s, 3H), 4.62 (br s, 2H), 5.32 (s, 2H), 6.66 (s, 1H), 7.30-7.39 (m, 1H), 7.41-7.50 (m, 2H), 7.65 (d, J=7.2 Hz, 1H).

### Intermediate 61

2-[4-Fluoro-3-(trifluoromethyl)phenyl]-2-oxoethyl 2-amino-5-methylthiophene-3-carboxylate

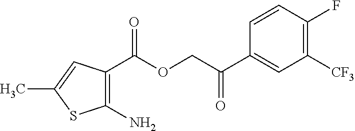 [SMILES: Cc1cc(C(=O)OCC(=O)c2ccc(F)c(C(F)(F)F)c2)c(N)s1]

The titled compound was prepared by the reaction of 2-amino-5-methylthiophene-3-carboxylic acid (550 mg, 3.49 mmol) with 2-bromo-144-fluoro-3-(trifluoromethyl)phenyllethanone (998 mg, 3.49 mmol) using potassium fluoride (305 mg, 5.24 mmol) in dry DMF (6.0 mL) as per the procedure described in Step 4 of Intermediate 1 to yield 817 mg of the product as a solid. ¹H NMR (300 MHz, DMSO-d₆): δ 2.20 (s, 3H), 5.57 (s, 2H), 6.56 (s, 2H), 7.20 (s, 2H), 7.74 (d, J=9.3 Hz, 1H), 8.31 (d, J=6.3 Hz, 1H).

### Intermediate 62

(2E)-3-(2-Chlorophenyl)-1-[4-hydroxy-5-methyl-1-(propan-2-yl)-1H-pyrazol-3-yl]prop-2-en-1-one

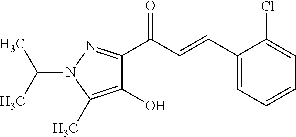 [SMILES: Cc1c(O)c(C(=O)/C=C/c2ccccc2Cl)nn1C(C)C]

### Step 1: 1-[4-Hydroxy-5-methyl-1-(propan-2-yl)-1H-pyrazol-3-yl]ethanone

To a stirred solution of isopropylhydrazine hydrochloride (1.0 g, 9.04 mmol) in water (11 mL) was added aqueous pyruvic aldehyde solution (40%, 10 mL, 54.29 mmol) followed by acetic acid (776 μL, 13.57 mmol) at RT. The reaction mixture was stirred at 110° C. for 3 h. The mixture was cooled to RT and extracted with ethyl acetate (100 mL×2). The organic layers were collected, dried over anhydrous sodium sulfate and concentrated under reduced pressure to obtain 715 mg of the titled product as oil. ¹H NMR (300 MHz, CDCl₃): δ 1.47 (d, J=6.3 Hz, 6H), 2.20 (s, 3H), 2.52 (s, 3H), 4.39-4.46 (m, 1H), 7.81 (s, 1H); APCI (m/z) 182 (M)⁺.

### Step 2: (2E)-3-(2-Chlorophenyl)-1-[4-hydroxy-5-methyl-1-(propan-2-yl)-1H-pyrazol-3-yl]prop-2-en-1-one

To a stirred solution of Step 1 intermediate (360 mg, 1.97 mmol) in ethanol (3.5 mL), 2-chlorobenzaldehyde (277 mg, 1.97 mmol) and a solution of sodium hydroxide (339 mg, 8.49 mmol) in ethanol (3.5 mL) were added. The reaction mixture was stirred for 18 h at RT. The solvent was evaporated under reduced pressure. The residue was diluted with water (10 mL) and extracted with ethyl acetate (50 mL×2). The organic layer was dried over anhydrous sodium sulfate and concentrated to obtain 340 mg of the titled product as oil. ¹H NMR (300 MHz, CDCl₃): δ 1.52 (d, J=6.3 Hz, 6H), 2.24 (s, 3H), 4.43-4.48 (m, 1H), 7.26 (s, 2H), 7.30-7.33 (m, 1H), 7.60 (s, 1H), 7.83-7.87 (m, 1H), 8.48 (s, 1H).

### Intermediate 63

(2E)-3-(2-Chlorophenyl)-1-(1-ethyl-4-hydroxy-5-methyl-1H-pyrazol-3-yl)prop-2-en-1-one

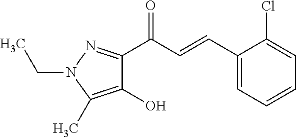 [SMILES: CCn1nc(C(=O)/C=C/c2ccccc2Cl)c(O)c1C]

To a stirred solution of 1-(1-ethyl-4-hydroxy-5-methyl-1H-pyrazol-3-yl)ethanone (500 mg, 2.97 mmol) and 2-chlorobenzaldehyde (350 μL, 2.97 mmol) in ethanol (5.0 mL) was added a solution of sodium hydroxide (511 mg, 12.79 mmol) in ethanol (5.0 mL) at RT and the mixture was stirred overnight. The solvent was evaporated under reduced pressure and the residue was acidified with HCl till pH 3. The aqueous mixture was extracted with ethyl acetate (50 mL×2) and the organic layer was washed with water (100 mL×2). The organic solution was concentrated under reduced pressure and the obtained residue was purified by silica gel column chromatography to afford 541 mg of the titled product as a solid. ¹H NMR (300 MHz, CDCl₃): δ 1.46 (t, J=7.5 Hz, 3H), 2.24 (s, 3H), 4.12 (q, J=7.5 Hz, 2H), 7.30-7.32 (m, 2H), 7.40-7.45 (m, 1H), 7.63-7.75 (m, 1H), 7.82-7.87 (m, 1H), 8.20 (br s, 1H), 8.35-8.40 (m, 1H).

### Intermediate 64

6-(2-Chlorophenyl)-5-methoxy-1,3-dimethylpyrano [2,3-c] pyrazol-4(1H)-one

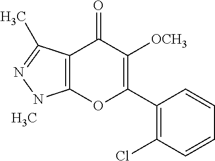

### Step 1: 1-(5-Hydroxy-1,3-dimethyl-1H-pyrazol-4-yl)-2-methoxyethanone

To a stirred mixture of 2,5-dimethyl-2,4-dihydro-3H-pyrazol-3-one (2.6 g, 23.18 mmol) and calcium hydroxide (6.9 g, 92.74 mmol) in dry 1,4 dioxane (60 mL), methoxy acetyl chloride (2.13 ml, 23.187 mmol) was added at RT. The reaction mixture was stirred at 100° C. for 18 h. The mixture was cooled to RT and acidified with 1 N HCl till pH 2-3. The mixture was extracted in ethyl acetate (200 mL×2), dried over anhydrous sodium sulfate and concentrated. The solid obtained was crystallized with ethyl acetate (10 mL) and n-hexane (5.0 mL) to yield 2.2 g of the titled product. ¹H NMR (300 MHz, CDCl₃): δ 2.36 (s, 3H), 3.52 (s, 3H), 3.59 (s, 3H), 4.36 (s, 2H).

### Step 2: Ethyl 2-chlorobenzoate

To a stirred solution of 2-chlorobenzoic acid (6.0 g, 38.32 mmol) in ethanol (60 mL), a catalytic amount of sulfuric acid was added at RT and the reaction mixture was stirred overnight. The solvent was recovered under reduced pressure and the residue was basified with saturated aqueous sodium bicarbonate solution (60 mL). The mixture was extracted with ethyl acetate (200 mL×3). The organic layer was dried over anhydrous sodium sulfate and concentrated to afford 6.4 g of the titled compound as oil. ¹H NMR (300 MHz, CDCl₃): δ 1.40 (t, J=7.5 Hz, 3H), 4.40 (q, J=6.6 Hz, 2H), 7.26-7.34 (m, 1H), 7.42 (q, J=9.3 Hz, 2H), 7.81 (d, J=7.2 Hz, 1H).

### Step 3: 1-(2-Chlorophenyl)-3-(5-hydroxy-1,3-dimethyl-1H-pyrazol-4-yl)-2-methoxypropane-1,3-dione

To a stirred suspension of sodium hydride (60% w/w, 800 mg, 20.01 mmol) in THF (10 mL), a solution of Step 1 intermediate (920 mg, 5.00 mmol) in THF (10 mL) was added at RT and the mixture was stirred at 50° C. for 30 min. Step 2 intermediate (1.28 g, 7.02 mmol) was added to the reaction mixture and stirred overnight at 60° C. The mixture was cooled to RT, acidified with 1 N HCl till pH 2-3 and extracted with chloroform (100 mL×4). The organic extract was dried over anhydrous sodium sulfate, filtered and concentrated. The obtained product was purified by silica gel column chromatography to yield 447 mg of the titled compound as a solid. ¹H NMR (300 MHz, CDCl₃): δ 2.32 (s, 3H), 3.59 (s, 3H), 3.62 (s, 3H), 5.47 (s, 1H), 7.34-7.38 (m, 1H), 7.40-7.45 (m, 2H), 7.50 (d, J=7.8 Hz, 1H).

### Step 4: 6-(2-Chlorophenyl)-5-methoxy-1,3-dimethylpyrano[2,3-c]pyrazol-4(1H)-one

A solution of Step 3 intermediate (435 mg, 1.35 mmol) in a mixture of sulfuric acid and acetic acid (5.5 mL, 1:10) was stirred overnight at 120° C. The mixture was cooled to RT and diluted with water (30 mL). The product was extracted in ethyl acetate (50 mL×3) and the combined organic extracts were dried over anhydrous sodium sulfate. The solution was filtered, concentrated and the residue obtained was purified by silica gel column chromatography to yield 207 mg of the titled compound as a solid. ¹H NMR (300 MHz, CDCl₃): δ 2.58 (s, 3H), 3.77-3.82 (m, 6H), 7.40-7.56 (m, 4H); ESI (m/z) 305 (M+H)⁺.

### Intermediate 65

6-(2-Chlorophenyl)-1-(4-fluorophenyl)-5-methoxy-3-methylpyrano[2,3-c]pyrazol-4(1H)-one

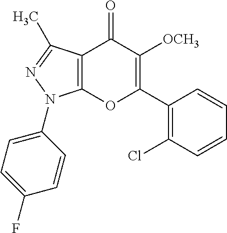 [SMILES: COc1c(-c2ccccc2Cl)oc2c(c(C)nn2-c2ccc(F)cc2)c1=O]

### Step 1: 2-(4-Fluorophenyl)-5-methyl-2,4-dihydro-3H-pyrazol-3-one

The titled compound was prepared by the reaction of ethyl acetoacetate (6.3 g, 48.40 mmol) with N,N′-diisopropylethylamine (8.0 mL, 49.12 mmol) and 4-fluorophenylhydrazine (7.87 g, 48.40 mmol) in ethanol (35 mL) at RT as per the procedure described in Step 2 of Intermediate 1 to yield 4.83 g of the titled compound as a solid. ¹H NMR (300 MHz, DMSO-d₆): δ 2.08 (s, 3H), 5.33 (s, 1H), 7.24 (t, J=9.3 Hz, 2H), 7.69-7.72 (m, 2H), 11.50 (br s, 1H); ESI (m/z) 193 (M+H)⁺.

### Step 2: 1-[1-(4-Fluorophenyl)-5-hydroxy-3-methyl-1H-pyrazol-4-yl]-2-methoxyethanone

The titled compound was prepared by the reaction of Step 1 intermediate (4.3 g, 22.37 mmol) with methoxy acetylchloride (2.0 mL) in the presence of calcium hydroxide (6.63 g, 89.49 mmol) in dry 1,4 dioxane (60 mL) as per the procedure described in Step 1 of intermediate 64 to afford 3.8 g of the desired product as a solid. ¹H NMR (300 MHz, CDCl₃): δ 2.44 (s, 3H), 3.55 (s, 3H), 4.43 (s, 3H), 7.14 (t, J=8.7 Hz, 2H), 7.74-7.78 (m, 2H).

### Step 3: 1-(2-Chlorophenyl)-3-[1-(4-fluorophenyl)-5-hydroxy-3-methyl-1H-pyrazol-4-yl]-2-methoxypropane-1,3-dione

To a stirred suspension of sodium hydride (60% w/w, 1.21 g, 50.42 mmol) in THF (38 mL), a solution of Step 2 intermediate (2.0 g, 7.56 mmol) in THF (10 mL) was added at RT and the reaction mixture was stirred at 50° C. for 30 min. The ethyl 2-chlorobenzoate (1.94 g, 10.54 mmol) was added to the mixture and the reaction was stirred overnight at 60° C. The reaction mixture was cooled to RT, acidified with 1 N HCl till pH 2-3 and extracted with ethyl acetate (150 mL×3). The organic extract was dried under anhydrous sodium sulfate, filtered and concentrated. The residue obtained was purified by silica gel column chromatography to yield 1.44 g of the titled compound as a solid. ¹H NMR (300 MHz, CDCl₃): δ 2.54 (s, 3H), 3.64 (s, 3H), 7.14 (t, J=8.7 Hz, 2H), 7.35-7.58 (m, 4H), 7.72-7.78 (m, 2H), 15.26 (br s, 1H); ESI (m/z) 401 (M−H)⁻.

### Step 4: 6-(2-Chlorophenyl)-1-(4-fluorophenyl)-5-methoxy-3-methylpyrano[2,3-c]pyrazol-4(1H)-one

To Step 3 intermediate (1.4 g, 3.47 mmol), a mixture of sulfuric acid and acetic acid (14 mL, 1:10) was added at RT. The reaction mixture was stirred overnight at 120° C. The mixture was diluted with water (100 mL) and extracted with ethyl acetate (250 mL×3). The organic extract was dried over anhydrous sodium sulfate and concentrated to yield 278 mg of the titled product as a solid. ¹H NMR (300 MHz, CDCl₃): δ 2.67 (s, 3H), 3.80 (s, 3H), 7.15 (t, J=8.7 Hz, 2H), 7.40-7.58 (m, 4H), 7.78-7.81 (m, 2H); ESI (m/z) 385 (M)⁺.

### Intermediate 66

2-(2-Chlorophenyl)-2-oxoethyl 5-amino-2-methyl-1,3-thiazole-4-carboxylate

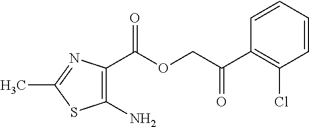 [SMILES: Cc1nc(C(=O)OCC(=O)c2ccccc2Cl)c(N)s1]

### Step 1: Ethyl 2-acetamido-2-cyanoacetate

To a stirred solution of ethyl cyanoglycoxalate-2-oxime (10 g, 70.37 mmol) in glacial acetic acid (50 mL), acetic anhydride (18 mL, 190 mmol) followed by zinc dust (14.8 g, 225.8 mmol) was added and reaction mixture was stirred at RT for 2 h. The mixture was concentrated under reduced pressure, neutralized with saturated solution of sodium bicarbonate and extracted with ethyl acetate (100 mL×3). The organic layer was dried over anhydrous sodium sulfate and concentrated under reduced pressure. The residue obtained was stirred with hexane and filtered to give 2.3 g of the titled product as a solid. ¹H NMR (300 MHz, DMSO-d₆): δ 1.21 (t, J=6.9 Hz, 3H), 1.92 (s, 3H), 4.19 (q, J=7.2 Hz, 2H), 5.71 (d, J=7.2 Hz, 1H), 9.18 (d, J=7.2 Hz, 1H); APCI (m/z) 171 (M+H)⁺.

### Step 2: Ethyl 5-amino-2-methyl-1,3-thiazole-4-carboxylate

The Lawesson's reagent (2.85 g, 7.05 mmol) was added to a stirred solution of Step 1 intermediate (2.0 g, 11.75 mmol) in dry toluene (25 mL) at RT and the mixture was heated to 120° C. overnight. The mixture was cooled to RT and concentrated under reduced pressure. The residue obtained was purified by flash silica gel column chromatography to yield 1.01 g of the product as a solid. ¹H NMR (300 MHz, DMSO-d₆): δ 1.24 (t, J=7.2 Hz, 3H), 2.38 (s, 3H), 4.18 (q, J=7.2 Hz, 2H), 7.19 (br s, 2H); APCI (m/z) 187 (M+H)⁺.

### Step 3: 5-Amino-2-methyl-1,3-thiazole-4-carboxylic acid

The titled compound was prepared by the ester hydrolysis of Step 2 intermediate (1.0 g, 5.36 mmol) using aqueous solution of potassium hydroxide (2.0 M, 6.4 mL, 8.05 mmol) in ethanol (18 mL) as per the procedure described in Step 3 of Intermediate 1 to yield 532 mg of the product as a solid. ¹H NMR (300 MHz, DMSO-d₆): δ 2.38 (s, 3H), 7.14 (brs, 2H); APCI (m/z) 159 (M+H)⁺.

### Step 4: 2-(2-chlorophenyl)-2-oxoethyl 5-amino-2-methyl-1,3-thiazole-4-carboxylate

The titled compound was prepared by the reaction of Step 3 intermediate (500 mg, 3.16 mmol) with 2-bromo-1-(2-chlorophenyl)ethanone (885 mg, 3.79 mmol) using potassium fluoride (275 mg, 4.74 mmol) in dry DMF (3.0 mL) as per the procedure described in Step 4 of Intermediate 1 to yield 483 mg of the product as a solid. ¹H NMR (300 MHz, DMSO-d₆): δ 2.39 (s, 3H), 5.35 (s, 2H), 7.31 (s, 2H), 7.49-7.59 (m, 3H), 7.78 (t, J=9.3 Hz, 1H); APCI (m/z) 311 (M+H)⁺.

### Intermediate 67

2-(2-Chlorophenyl)-2-oxoethyl 5-amino-2-(trifluoromethyl)-1,3-thiazole-4-carboxylate

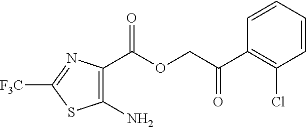 [SMILES: Nc1sc(C(F)(F)F)nc1C(=O)OCC(=O)c1ccccc1Cl]

### Step 1: Ethyl amino(cyano)acetate

To a suspension of ethyl cyanoglyoxylate-2-oxime (10 g, 70.37 mmol) in water (60 mL), a saturated aqueous sodium bicarbonate solution (30 mL) followed by the sodium dithionate (34.30 g, 97.01 mmol) were added at RT and the mixture was stirred for 30 min. The mixture was extracted with chloroform (200 mL×3). The organic layer was dried over anhydrous sodium sulfate and concentrated under reduced pressure to obtain 3.85 g of the titled product as oil. The product obtained was used immediately for next Step.

### Step 2: Ethyl cyano[(trifluoroacetyl)amino]acetate

To a stirred solution of Step 1 intermediate (3.75 g, 29.26 mmol) in dry THF (38 mL), dry pyridine (4.71 mL, 58.52 mmol) and trifluoroacetic anhydride (4.3 mL, 30.73 mmol) were added drop wise at RT. The mixture was stirred at RT overnight. The mixture was basified using aqueous saturated sodium bicarbonate and extracted with ethyl acetate (2×300 mL). The organic layer was washed with brine (200 mL) and dried over anhydrous sodium sulfate. The mixture was concentrated under reduced pressure to yield 4.01 g of the product as a solid. ¹H NMR (300 MHz, DMSO-d₆): δ 1.22 (t, J=7.2 Hz, 3H), 4.25 (q, J=7.5 Hz, 2H), 6.10 (d, J=7.2 Hz, 1H), 10.99 (d, J=6.9 Hz, 1H).

### Step 3: Ethyl 5-amino-2-(trifluoromethyl)-1,3-thiazole-4-carboxylate

To a stirred solution of Step 2 intermediate (4.9 g, 21.87 mmol) in toluene (60 mL), Lawesson's reagent (5.30 g, 13.12 mmol) was added at RT and the mixture was heated to 120° C. overnight. The mixture was cooled to RT and concentrated under reduced pressure. The residue was purified by flash silica gel column chromatography to yield 1.20 g of the product as a solid. ¹H NMR (300 MHz, DMSO-d₆): δ 1.28 (t, J=7.2 Hz, 3H), 4.26 (q, J=7.5 Hz, 2H), 7.89 (s, 2H).

### Step 4: 5-Amino-2-(trifluoromethyl)-1,3-thiazole-4-carboxylic acid

The titled compound was prepared by the ester hydrolysis of Step 3 intermediate (1.15 g, 4.79 mmol) using aqueous solution of potassium hydroxide (2.0 M, 5.7 mL, 7.18 mmol) in ethanol (16 mL) as per the procedure described in Step 3 of Intermediate 1 to yield 630 mg of the product as a solid. ¹H NMR (300 MHz, DMSO-d₆): δ 3.39 (br s, 2H), 7.81 (br s, 1H).

### Step 5: 2-(2-Chlorophenyl)-2-oxoethyl 5-amino-2-(trifluoro methyl)-1,3-thiazole-4-carboxylate

The titled compound was prepared by the reaction of Step 4 intermediate (620 mg, 2.92 mmol) with 2-bromo-1-(3-chlorophenyl)ethanone (820 mg, 3.50 mmol) using potassium fluoride (255 mg, 4.38 mmol) in dry DMF (6.0 mL) as per the procedure described in Step 4 of Intermediate 1 to yield 470 mg of the product as a solid. ¹H NMR (300 MHz, DMSO-d₆): δ 5.48 (s, 2H), 7.45-7.56 (m, 1H), 7.61 (d, J=4.5 Hz, 2H), 7.84 (d, J=7.8 Hz, 1H), 8.02 (s, 2H).

### Intermediate 68

2-(2-Chlorophenyl)-2-oxoethyl 5-amino-3-methyl-1,2-oxazole-4-carboxylate

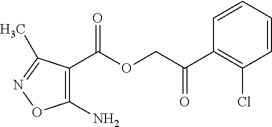 [SMILES: Cc1noc(N)c1C(=O)OCC(=O)c1ccccc1Cl]

### Step 1: 5-Amino-3-methyl-1,2-oxazole-4-carboxylic acid

To a stirred solution of ethyl 5-amino-3-methyl-1,2-oxazole-4-carboxylate (1.66 g, 9.76 mmol) in ethanol (30 mL), 1.25 M aqueous potassium hydroxide solution (8 mL, 14.64 mmol) was added at RT. The mixture was stirred overnight at 90° C. The organic solvent was recovered under reduced pressure and the residue was acidified with 1N HCl till pH-2. The precipitated solid was filtered, washed with water (100 mL) and dried under vacuum to obtain 850 mg of the titled compound as a solid. ¹H NMR (300 MHz, DMSO-d₆): δ 2.16 (s, 3H), 7.58 (br s, 2H); APCI (m/z) 143 (M+H)⁺.

### Step 2: 2-(2-Chlorophenyl)-2-oxoethyl 5-amino-3-methyl-1,2-oxazole-4-carboxylate

To a well stirred solution of Step 1 intermediate (840 mg, 5.91 mmol) in DMF (9.0 mL) were added potassium fluoride (516 mg, 8.86 mmol) and 2-bromo-1-(2-chlorophenyl)ethanone (1.66 g, 7.09 mmol) at RT. The mixture was stirred overnight at same temperature. The reaction was quenched with saturated aqueous sodium bicarbonate solution (10 mL). The precipitated solid was filtered, washed with water (50 mL) and dried under vacuum to afford 1.35 g of the titled compound as a solid. ¹H NMR (300 MHz, DMSO-d₆): δ 2.17 (s, 3H), 5.33 (s, 2H), 7.51-7.60 (m, 3H), 7.78 (d, J=7.2 Hz, 1H), 7.85-7.88 (m, 2H); APCI (m/z) 295 (M+H)⁺.

### Intermediate 69

2-[2-Chloro-4-(2-methoxyethoxy)phenyl]-2-oxoethyl 5-amino-1,3-dimethyl-1H-pyrazole-4-carboxylate

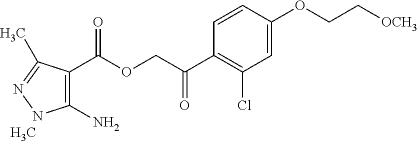

To a stirred solution of step-3 of intermediate 9 (250 mg, 1.61 mmol) in dry DMF (3 ml), 2-bromo-1-[2-chloro-4-(2-methoxyethoxy)phenyl]ethanone (490 mg, 1.61 mmol) was added followed by potassium fluoride (140 mg, 2.42 mmol) at RT and the reaction mixture was stirred overnight at RT. The reaction mixture was diluted with ethyl acetate (30 mL) and quenched with water (75 mL). The mixture was extracted with ethyl acetate (3×100 mL). The organic layer was washed with water (2×100 mL) and dried over anhydrous sodium sulfate. The solvent was distilled off under vacuum and the residue was purified by flash silica gel column chromatography to afford 360 mg of the product as a solid. ¹H NMR (300 MHz, DMSO-d₆): δ 2.14 (s, 3H), 3.30 (s, 3H), 3.46 (s, 3H), 3.66 (t, J=4.5 Hz, 2H), 4.21 (t, J=4.8 Hz, 2H), 5.29 (s, 2H), 6.24 (s, 2H), 7.05 (d, J=8.1 Hz, 1H), 7.16 (s, 1H), 7.84 (d, J=9.0 Hz, 1H); APCI (m/z) 380 (M−H)⁻.

### Intermediate 70

2-[2-Fluoro-4-(2-methoxyethoxy)phenyl]-2-oxoethyl 5-amino-1-methyl-3-(trifluoromethyl)-1H-pyrazole-4-carboxylate

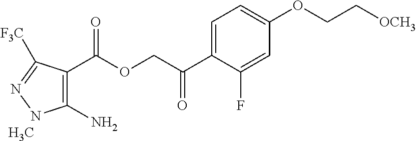

The titled compound is prepared by the reaction of 5-amino-1-methyl-3-(trifluoromethyl)-1H-pyrazole-4-carboxylic acid (800 mg, 3.82 mmol) with 2-bromo-1-[2-fluoro-4-(2-methoxyethoxy)phenyl]ethanone (1.11 g, 3.82 mmol) using potassium fluoride (333 mg, 5.73 mmol) in DMF (8.0 mL) as per the procedure described in Step 4 of Intermediate 1 to yield 1.18 g of the product as a solid. ¹H NMR (300 MHz, DMSO-d₆): δ 3.32 (s, 3H), 3.66-3.70 (m, 5H), 4.23 (t, J=4.5 Hz, 2H), 5.33 (s, 2H), 6.71 (s, 2H), 6.93-7.06 (m, 2H), 7.86 (t, J=7.6 Hz, 1H).

### Intermediate 71

2-(2,6-Difluoro-4-methoxyphenyl)-2-oxoethyl 5-amino-1,3-dimethyl-1H-pyrazole-4-carboxylate

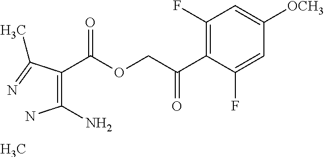

The titled intermediate was prepared by the reaction of 5-amino-1,3-dimethyl-1H-pyrazole-4-carboxylic acid (600 mg, 3.86 mmol) with 2-bromo-1-(2,6-difluoro-4-methoxyphenyl)ethanone (1.02 g, 3.86 mmol) using potassium fluoride (336 mg, 5.79 mmol) in dry DMF (6.0 mL) as per the procedure described in Step 4 of Intermediate 1 to yield 579 mg of the product as a solid. ¹H NMR (300 MHz, DMSO-d₆): δ 2.13 (s, 3H), 3.46 (s, 3H), 3.85 (s, 3H), 5.14 (s, 2H), 6.25 (s, 2H), 6.90 (d, J=8.4 Hz, 2H).

### Intermediate 72

2-(2,6-Difluorophenyl)-2-oxoethyl 5-amino-1-ethyl-3-methyl-1H-pyrazole-4-carboxylate

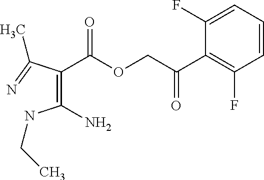 [SMILES: CCN/C(N)=C(\C(C)=N)C(=O)OCC(=O)c1c(F)cccc1F]

To a stirred solution of 5-amino-1-ethyl-3-methyl-1H-pyrazole-4-carboxylic acid (500 mg, 2.95 mmol) in dry DMF (5.0 ml), 2-bromo-1-(2,6-difluorophenyl)ethanone (693 mg, 2.95 mmol) and potassium fluoride (257 mg, 4.42 mmol) were added at RT. The mixture was stirred overnight at RT. The reaction mixture was quenched with water (75 mL) and extracted with ethyl acetate (3×100 mL). The organic layer was washed with water (2×100 mL) and dried over anhydrous sodium sulfate. The solvent was distilled off under vacuum and the residue obtained was purified by flash silica gel column chromatography to afford 912 mg of the desired product as a solid. ¹H NMR (300 MHz, DMSO-d₆): δ 1.17 (t, J=4.8 Hz, 3H), 2.10 (s, 3H), 3.82 (q, J=6.9 Hz, 2H), 5.17 (s, 2H), 6.25 (s, 2H), 7.24 (t, J=8.4 Hz, 2H), 7.62-7.69 (m, 1H); APCI (m/z) 322 (M−H)⁻.

### Intermediate 73

2-(2-Fluoro-4-methoxyphenyl)-2-oxoethyl 5-amino-1-ethyl-3-methyl-1H-pyrazole-4-carboxylate

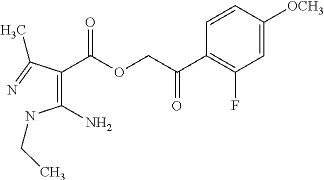 [SMILES: CCN/C(N)=C(\C(C)=N)C(=O)OCC(=O)c1ccc(OC)cc1F]

The titled compound was prepared by the reaction of 5-amino-1-ethyl-3-methyl-1H-pyrazole-4-carboxylic acid (600 mg, 3.54 mmol) with 2-bromo-1-(2-fluoro-4-methoxyphenyl)ethanone (875 mg, 3.54 mmol) using potassium fluoride (309 mg, 5.31 mmol) in dry DMF (6.0 mL) as per the procedure described in Step 4 of Intermediate 1 to yield 630 mg of the product as a solid. ¹H NMR (300 MHz, DMSO-d₆): δ 1.20 (t, J=4.5 Hz, 3H), 2.18 (s, 3H), 3.81-3.89 (m, 5H), 5.29 (s, 2H), 6.28 (s, 2H), 6.93-7.05 (m, 2H), 7.86 (t, J=8.1 Hz, 1H).

### Intermediate 74

2-(2-Fluoro-4-methoxyphenyl)-2-oxoethyl 5-amino-3-methyl-1-(2,2,2-trifluoroethyl)-1H-pyrazole-4-carboxylate

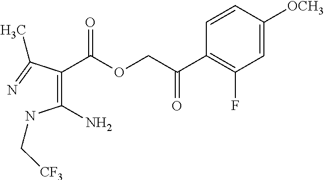 [SMILES: COc1ccc(C(=O)COC(=O)/C(C(C)=N)=C(\N)NCC(F)(F)F)c(F)c1]

The titled compound was prepared by the reaction of 5-amino-3-methyl-1-(2,2,2-trifluoroethyl)-1H-pyrazole-4-carboxylic acid (1.3 g, 5.82 mmol) with 2-bromo-1-(2-fluoro-4-methoxyphenyl)ethanone (1.43 g, 5.82 mmol) using potassium fluoride (507 mg, 8.73 mmol) in dry DMF (13 mL) as per the procedure described in Step 4 of Intermediate 1 to yield 910 mg of the product as a solid. ¹H NMR (300 MHz, DMSO-d₆): δ 2.19 (s, 3H), 3.87 (s, 3H), 4.81-4.89 (m, 2H), 5.31 (s, 2H), 6.69 (s, 2H), 6.94-7.04 (m, 2H), 7.85-7.89 (m, 1H); APCI (m/z) 388 (M−H)⁻.

### Intermediate 75

2-(2,6-Difluorophenyl)-2-oxoethyl 5-amino-3-ethyl-1-methyl-1H-pyrazole-4-carboxylate

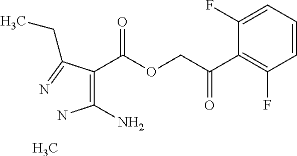

### Step 1: Ethyl 5-amino-3-ethyl-1-methyl-1H-pyrazole-4-carboxylate

To a stirred solution of ethyl (2Z)-2-cyano-3-ethoxypent-2-enoate (3.8 g, 19.3 mmol) in ethanol (38 mL) was added methyl hydrazine sulfate (2.8 g, 19.3 mmol) followed by N,N-diisopropylethylamine (6.6 mL, 38.6 mmol) at RT. The mixture was stirred overnight at 90° C. The ethanol was evaporated under reduced pressure and the residue was basified with aqueous saturated sodium bicarbonate solution (40 mL). The aqueous mixture was extracted with ethyl acetate (75 mL×2). The organic layer was dried over anhydrous sodium sulfate and concentrated under reduced pressure. The residue obtained was purified by silica gel column chromatography to yield 1.68 g of the product as a solid. ¹H NMR (300 MHz, DMSO-d₆): δ 1.09 (t, J=4.5 Hz, 3H), 1.24 (t, J=4.8 Hz, 3H), 2.57 (q, J=7.2 Hz, 2H), 3.45 (s, 3H), 4.15 (q, J=6.9 Hz, 2H), 6.14 (s, 2H).

### Step 2: 5-Amino-3-ethyl-1-methyl-1H-pyrazole-4-carboxylic acid

The titled compound was prepared by the ester hydrolysis of Step 2 intermediate (1.65 g, 8.36 mmol) using aqueous solution of potassium hydroxide (2.0 M, 6 mL, 16.73 mmol) in ethanol (17 mL) as per the procedure described in Step 3 of Intermediate 1 to yield 3.1 g of the product as a solid. ¹H NMR (300 MHz, DMSO-d₆): δ 1.06 (t, J=7.2 Hz, 3H), 2.30 (q, J=7.5 Hz, 2H), 3.40 (s, 3H), 4.99 (s, 2H), 5.07 (s, 1H).

### Step 3: 2-(2,6-Difluorophenyl)-2-oxoethyl 5-amino-3-ethyl-1-methyl-1H-pyrazole-4-carboxylate

The titled compound was prepared by the reaction of Step 2 intermediate (1.0 g, 5.91 mmol) with 2-bromo-1-(2,6-difluorophenyl)ethanone (1.39 g, 5.91 mmol) using potassium fluoride (525 mg, 8.87 mmol) in dry DMF (10 mL) as per the procedure described in Step 4 of Intermediate 1 to afford 840 mg of the product as a solid. ¹H NMR (300 MHz, DMSO-d₆): δ 1.06 (t, J=6.6 Hz, 3H), 2.53 (q, J=6.6 Hz, 2H), 3.46 (s, 3H), 5.18 (s, 2H), 6.25 (s, 2H), 7.25 (t, J=8.1 Hz, 2H), 7.63-7.68 (m, 1H).

### Intermediate 76

2-(2-Fluoro-4-methoxyphenyl)-2-oxoethyl 5-amino-3-ethyl-1-methyl-1H-pyrazole-4-carboxylate

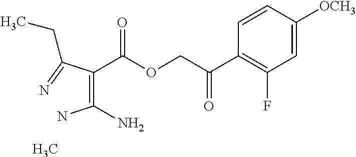

The titled compound was prepared by the reaction of 5-amino-3-ethyl-1-methyl-1H-pyrazole-4-carboxylic acid (1.5 g, 8.87 mmol) with 2-bromo-1-(2-fluoro-4-methoxyphenyl)ethanone (2.2 g, 8.87 mmol) using potassium fluoride (775 mg, 13.30 mmol) in dry DMF (15 mL) as per the procedure described in Step 4 of Intermediate 1 to yield 1.7 g of the product as a solid. ¹H NMR (300 MHz, DMSO-d₆): δ 1.11 (t, J=6.6 Hz, 3H), 2.60 (q, J=6.6 Hz, 2H), 3.48 (s, 3H), 3.87 (s, 3H), 5.29 (s, 2H), 6.27 (s, 2H), 6.92-7.03 (m, 2H), 7.87 (t, J=8.1 Hz, 1H); ESI (m/z) 336 (M+H)⁺.

### Intermediate 77

2-(2-Chloro-4-methoxyphenyl)-2-oxoethyl 5-amino-3-ethyl-1-methyl-1H-pyrazole-4-carboxylate

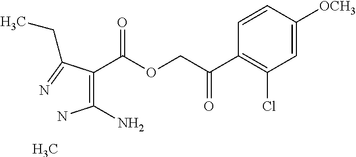

The titled compound was prepared by the reaction of 5-amino-3-ethyl-1-methyl-1H-pyrazole-4-carboxylic acid (700 mg, 4.14 mmol) with 2-bromo-1-(2-chloro-4-methoxyphenyl)ethanone (1.1 g, 4.14 mmol) using potassium fluoride (360 mg, 6.21 mmol) in dry DMF (7.0 mL) as per the procedure described in Step 4 of Intermediate 1 to yield 900 mg of the product as a solid. ¹H NMR (300 MHz, DMSO-d₆): δ 1.07 (t, J=6.6 Hz, 3H), 2.55 (q, J=6.6 Hz, 2H), 3.46 (s, 3H), 3.83 (s, 3H), 5.30 (s, 2H), 6.24 (s, 2H), 6.99-7.04 (m, 2H), 7.83 (d, J=8.1 Hz, 1H); ESI (m/z) 352 (M)⁺.

### Intermediate 78

2-(2,6-Difluorophenyl)-2-oxoethyl 5-amino-3-methyl-1-(propan-2-yl)-1H-pyrazole-4-carboxylate

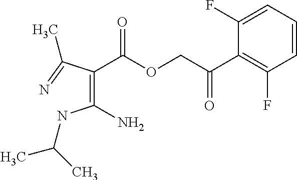 [SMILES: CC(=N)/C(C(=O)OCC(=O)c1c(F)cccc1F)=C(/N)NC(C)C]

The titled compound was prepared by the reaction of 5-amino-3-methyl-1-(propan-2-yl)-1H-pyrazole-4-carboxylic acid (800 mg, 4.36 mmol) with 2-bromo-1-(2,6-difluorophenyl)ethanone (1.02 g, 4.36 mmol) using potassium fluoride (380 mg, 6.54 mmol) in dry DMF (8.0 mL) as per the procedure described in Step 4 of Intermediate 1 to yield 252 mg of the product as a solid. ¹H NMR (300 MHz, DMSO-d₆): δ 1.27 (d, J=6.3 Hz, 6H), 2.13 (s, 3H), 4.36-4.42 (m, 1H), 5.19 (s, 2H), 6.28 (s, 2H), 7.23-7.30 (m, 2H), 7.63-7.70 (m, 1H).

### Intermediate 79

2-(2-chlorophenyl)-2-oxoethyl 3-amino-5-isopropyl-1-methyl-1H-pyrazole-4-carboxylate

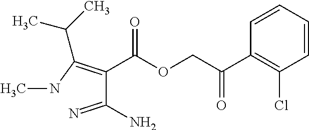 [SMILES: CN/C(=C(\C(=N)N)C(=O)OCC(=O)c1ccccc1Cl)C(C)C]

### Step 1: Ethyl (2Z)-2-cyano-3-hydroxy-4-methylpent-2-enoate

To a suspension of sodium hydride (60% w/w, 3.75 g, 93.84 mmol) in dry THF (78 mL), ethylcyanoacetate (5 mL, 46.92 mmol) was added at 0° C. The reaction mixture was stirred for 1 h at RT. The reaction mixture was cooled to 0° C. and to it isobutyryl chloride (5.0 g, 46.92 mmol) was added. The reaction mixture was stirred at RT for 18 h. The mixture was quenched with 2N sulfuric acid and extracted with ethyl acetate (250 mL×3). The organic extract was dried under anhydrous sodium sulfate, filtered and concentrated. The obtained product was purified by silica gel column chromatography to yield 8.9 g of the title compound as oil. ¹H NMR (300 MHz, CDCl₃): δ 1.24 (d, J=6.6 Hz, 6H), 1.37 (t, J=6.6 Hz, 3H), 3.08-3.17 (m, 1H), 4.34 (q, J=6.6 Hz, 2H), 13.78 (s, 1H).

### Step 2: Ethyl (2Z)-3-chloro-2-cyano-4-methylpent-2-enoate

To a stirred solution of the reaction of Step 1 intermediate (8.9 g, 48.58 mmol) in dichloromethane (90 mL), phosphorous oxychloride (4.8 mL, 53.43 mmol) and triethylamine (10.15 mL, 72.87 mmol) were added at RT. The reaction mixture was heated to 50° C. and stirred at the 50° C. for 18 h. The reaction mixture was cooled to RT and quenched with saturated sodium bicarbonate solution (200 mL). The layer was separated and the aqueous layer was extracted with dichloromethane (3×250 mL). The organic layer was dried over anhydrous sodium sulfate, filtered and concentrated under reduced pressure. The obtained product was purified by silica gel column chromatography to afford 4.1 g of the titled product as oil. ¹H NMR (300 MHz, CDCl₃): δ 1.25 (d, J=6.6 Hz, 6H), 1.38 (t, J=6.6 Hz, 3H), 3.08-3.16 (m, 1H), 4.34 (q, J=6.6 Hz, 2H).

### Step 3: 3-amino-5-isopropyl-1-methyl-1H-pyrazole-4-carboxylic acid

A mixture of step 2 intermediate (4 g, 19.83 mmol) and methyl hydrazine (913 μL, 19.83 mmol) in 10% NaOH (40 ml) was stirred at RT for overnight. The reaction mixture was cooled with ice, acidified with 1 N HCl and thesolid obtained was collected by filtration to yield 1.3 of the product. ¹H NMR (300 MHz, DMSO-d₆): δ 1.27 (d, J=6.0 Hz, 6H), 3.10-3.19 (m, 1H), 3.60 (s, 3H), 11.11 (br s, 1H); APCI (m/z) 184 (M+H)⁺.

### Step 4: 2-(2-chlorophenyl)-2-oxoethyl 3-amino-5-isopropyl-1-methyl-1H-pyrazole-4-carboxylate

The title compound was prepared by the reaction of step 3 intermediate (600 mg, 3.27 mmol) with 2-bromo-1-(2-chlorophenyl)ethanone (761 mg, 3.27 mmol) using potassium fluoride (285 mg, 4.91 mmol) in dry DMF (6.0 mL) as per the procedure described in step 4 of intermediate 1 to yield 545 mg of the product as solid. ¹H NMR (300 MHz, DMSO-d₆): δ 1.27 (d, J=6.2 Hz, 6H), 3.11-3.23 (m, 1H), 3.63 (s, 3H), 5.40 (s, 2H), 7.45-7.52 (m, 1H), 7.57 (s, 2H), 7.76 (d, J=7.2 Hz, 1H); ESI (m/z) 335 (M)⁺.

### Intermediate 80

2-(2,6-Difluorophenyl)-2-oxoethyl 5-amino-1-benzyl-3-methyl-1H-pyrazole-4-carboxylate

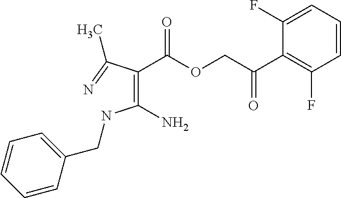 [SMILES: CC(=N)/C(C(=O)OCC(=O)c1c(F)cccc1F)=C(/N)NCc1ccccc1]

### Step 1: Ethyl 5-amino-1-benzyl-3-methyl-1H-pyrazole-4-carboxylate

To a stirred solution of ethyl (2Z)-2-cyano-3-ethoxybut-2-enoate (5.0 g, 27.3 mmol) in ethanol (50 mL), benzylhydrazine dihydrochloride (5.3 g, 27.3 mmol) followed by N, N-diisopropylethylamine (14 mL, 81.9 mmol) were added at RT. The reaction mixture was stirred overnight at 90° C. The ethanol was removed by evaporation and the residue obtained was basified with aqueous saturated sodium bicarbonate solution (50 mL). The aqueous mixture was extracted with ethyl acetate (100 mL×2). The organic layer was dried over anhydrous sodium sulfate and concentrated under reduced pressure. The residue obtained was purified by silica gel column chromatography to yield 4.45 g of the titled product as a solid. ¹H NMR (300 MHz, DMSO-d₆): δ 1.25 (t, J=6.6 Hz, 3H), 2.16 (s, 3H), 4.16 (q, J=6.6 Hz, 2H), 5.09 (s, 2H), 6.32 (s, 2H), 7.14 (d, J=8.1 Hz, 2H), 7.23-7.35 (m, 3H).

### Step 2: 5-Amino-1-benzyl-3-methyl-1H-pyrazole-4-carboxylic acid

The titled intermediate was prepared by the ester hydrolysis of Step 1 intermediate (4.4 g, 16.9 mmol) using aqueous solution of potassium hydroxide (2.0 M, 20 mL, 33.96 mmol) as per the procedure described in Step 3 of Intermediate 1 to yield 3.3 g of the product as a solid. ¹H NMR (300 MHz, DMSO-d₆): δ 2.15 (s, 3H), 5.07 (s, 2H), 6.30 (s, 2H), 7.15 (d, J=7.8 Hz, 2H), 7.23-7.36 (m, 3H), 11.77 (br s, 1H); ESI (m/z) 232 (M+H)⁺.

### Step 3: 2-(2,6-Difluorophenyl)-2-oxoethyl 5-amino-1-benzyl-3-methyl-1H-pyrazole-4-carboxylate

The titled compound was prepared by the reaction of Step 2 intermediate (1.0 g, 4.32 mmol) with 2-bromo-1-(2,6-difluorophenyl)ethanone (1.05 g, 4.32 mmol) using potassium fluoride (380 mg, 6.48 mmol) in dry DMF (10 mL) as per the procedure described in Step 4 of Intermediate 1 to afford 1.45 g of the product as a solid. ¹H NMR (300 MHz, DMSO-d₆): δ 2.13 (s, 3H), 5.10 (s, 2H), 5.20 (s, 2H), 6.45 (s, 2H), 7.14 (d, J=8.1 Hz, 2H), 7.22-7.35 (m, 5H), 7.64-7.69 (m, 1H); ESI (m/z) 386 (M+H)⁺.

### Intermediate 81

2-(2-Fluoro-4-methoxyphenyl)-2-oxoethyl 5-amino-1-benzyl-3-methyl-1H-pyrazole-4-carboxylate

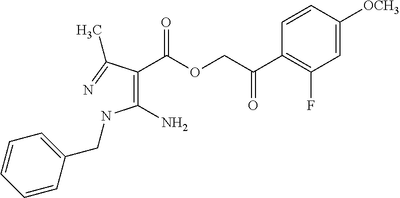 [SMILES: COc1ccc(C(=O)COC(=O)/C(C(C)=N)=C(\N)NCc2ccccc2)c(F)c1]

The titled compound was prepared by the reaction of 5-amino-1-benzyl-3-methyl-1H-pyrazole-4-carboxylic acid (1.0 g, 4.32 mmol) with 2-bromo-1-(2-fluoro-4-methoxyphenyl)ethanone (1.07 g, 4.32 mmol) using potassium fluoride (380 mg, 6.48 mmol) in dry DMF (10 mL) as per the procedure described in Step 4 of Intermediate 1 to yield 1.4 g of the product as a solid. ¹H NMR (300 MHz, DMSO-d₆): δ 2.19 (s, 3H), 3.87 (s, 3H), 5.11 (s, 2H), 5.30 (s, 2H), 6.46 (s, 2H), 6.94-7.04 (m, 2H), 7.17 (d, J=8.7 Hz, 2H), 7.25-7.37 (m, 3H), 7.87 (t, J=8.7 Hz, 1H); ESI (m/z) 398 (M)⁺.

### Intermediate 82

2-(2-Chlorophenyl)-2-oxoethyl 4-amino-3-(cyclopropylcarb amoyl)-1,2-thiazole-5-carboxylate

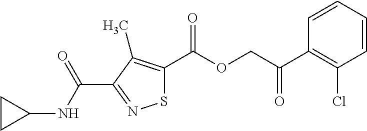 [SMILES: Cc1c(C(=O)NC2CC2)nsc1C(=O)OCC(=O)c1ccccc1Cl]

### Step 1: (2E)-2-Cyano-N-cyclopropyl-2-(hydroxyimino)ethanamide

A solution of sodium nitrite (6.94 g, 100.6 mmol) in water (70 mL) at 5-10° C., a stirred solution of 2-cyano-N-cyclopropylacetamide (5.0 g, 40.27 mmol) in acetic acid (10 mL) was added drop-wise. The reaction was stirred at 10° C. for 8 h and then at RT overnight. The precipitated solid was filtered and dried well to obtain 1.53 g of the titled product. ¹H NMR (300 MHz, DMSO-d₆): δ 0.56-0.68 (m, 4H), 2.71-2.75 (m, 1H), 8.59 (s, 1H), 14.64 (br s, 1H).

### Step 2: (2E)-2—Cyano-N-cyclopropyl-2-({[(4-methylphenyl)sulfonyl] oxy} iminoethanamide

To a stirred solution of Step 1 intermediate (1.5 g, 9.79 mmol) in pyridine (4.0 mL) at 5° C. was added tosyl chloride (2.01 g, 10.57 mmol) portion wise and the resulting mixture was stirred overnight at RT. Ethanol (15 mL) was added to the above mixture, the precipitated solid was filtered and dried well to afford 1.69 g of the desired product as a solid. ¹H NMR (300 MHz, DMSO-d₆): δ 0.60-0.72 (m, 4H), 2.44 (s, 3H), 2.70-2.75 (m, 1H), 7.54 (d, J=8.7 Hz, 2H), 7.99 (d, J=8.7 Hz, 2H), 8.98 (s, 1H).

### Step 3: Ethyl 4-amino-3-(cyclopropylcarbamoyl)-1,2-thiazole-5-carboxylate

To a stirred solution of Step 2 intermediate (700 mg, 2.27 mmol) and ethyl-2-mercaptoacetate (300 μL, 2.73 mmol) in ethanol (3.0 mL) at 0° C. was added morpholine (300 μL, 3.41 mmol). The resulting mixture was stirred for 30 min. The mixture was diluted with water (10 mL) and extracted with ethyl acetate (150 mL×4). The organic layer was dried over anhydrous sodium sulfate and concentrated under reduced pressure. The residue was purified by silica gel column chromatography to afford 678 mg of the product as a solid. ¹H NMR (300 MHz, CDCl₃): δ 0.66 (q, J=6.0 Hz, 2H), 0.85 (q, J=6.0 Hz, 2H), 1.34 (t, J=6.8 Hz, 3H), 2.81-2.88 (m, 1H), 4.34 (q, J=7.2 Hz, 2H), 7.26 (s, 2H); ESI (m/z) 256 (M+H)⁺.

### Step 4: 4-Amino-3-(cyclopropylcarbamoyl)-1,2-thiazole-5-carboxylic acid

To a stirred solution of Step 3 intermediate (650 mg, 2.54 mmol) in ethanol (2.5 mL), potassium hydroxide solution (213 mg, 3.81 mmol) in water (2.5 mL) was added. The resulting mixture was refluxed for 3 h. the mixture was concentrated under reduced pressure, cooled to 0° C. and acidified with 1 N HCl till pH 2. The precipitated solid was filtered and dried well to yield 462 mg of the titled product as a solid. ¹H NMR (300 MHz, DMSO-d₆): δ 0.61-0.67 (m, 4H), 2.79-2.83 (m, 1H), 3.61 (br s, 2H), 7.59-7.64 (m, 1H), 8.77 (s, 1H); ESI (m/z) 226 (M−H)⁻.

### Step: 5: 2-(2-Chlorophenyl)-2-oxoethyl 4-amino-3-(cyclopropylcarbamoyl)-1,2-thiazole-5-carboxylate

The titled compound was prepared by the reaction of Step 4 intermediate (450 mg, 1.98 mmol) with 2-bromo-1-(2-chlorophenyl)ethanone (552 mg, 2.37 mmol) using potassium fluoride (172 mg, 2.97 mmol) in dry DMF (5.0 mL) as per the procedure described in Step 4 of Intermediate 1 to yield 565 mg of the product as a solid. ¹H NMR (300 MHz, CDCl₃): δ 0.67 (q, J=6.0 Hz, 2H), 0.85 (q, J=6.0 Hz, 2H), 2.81-2.86 (m, 1H), 5.41 (s, 2H), 6.74 (br s, 2H), 7.26 (s, 1H), 7.36-7.46 (m, 2H), 7.66 (t, J=8.7 Hz, 1H); ESI (m/z) 380 (M+H)⁺.

### Intermediate 83

2-(2,6-Difluorophenyl)-2-oxoethyl 5-amino-3-methyl-1-propyl-1H-pyrazole-4-carboxylate

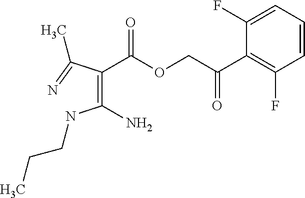 [SMILES: CCCN/C(N)=C(\C(C)=N)C(=O)OCC(=O)c1c(F)cccc1F]

### Step 1: Ethyl 5-amino-3-methyl-1-propyl-1H-pyrazole-4-carboxylate

The titled compound was prepared by the reaction of ethyl (2E)-2-cyano-3-ethoxybut-2-enoate (2.85 g, 15.6 mmol) with propylhydrazine (1.38 g, 18.68 mmol) using N,N′-DIPEA (5.5 mL, 31.2 mmol) in ethanol (30 mL) as per the procedure described in Step 1 of Intermediate 75 to yield 3.15 g of the product as oil. ¹H NMR (300 MHz, CDCl₃): δ 0.94 (t, J=7.2 Hz, 3H), 1.32 (t, J=6.9 Hz, 3H), 1.82 (q, J=7.2 Hz, 2H), 2.36 (s, 3H), 3.82 (t, J=8.4 Hz, 2H), 4.27 (q, J=7.5 Hz, 2H), 5.15 (br s, 2H); ESI (m/z) 212 (M+H)⁺.

### Step 2: 5-Amino-3-methyl-1-propyl-1H-pyrazole-4-carboxylic acid

The titled intermediate was prepared by the ester hydrolysis of Step 1 intermediate (3.1 g, 14.7 mmol) using aqueous solution of potassium hydroxide (2.0 M, 20 mL, 29.44 mmol) as per the procedure described in Step 3 of Intermediate 1 to yield 1.25 g of the product as a solid. ¹H NMR (300 MHz, DMSO-d₆): δ 0.81 (t, J=7.2 Hz, 3H), 1.63 (q, J=6.9 Hz, 2H), 2.13 (s, 3H), 3.73 (t, J=7.2 Hz, 2H), 6.12 (br s, 2H).

### Step 3: 2-(2,6-Difluorophenyl)-2-oxoethyl 5-amino-3-methyl-1-propyl-1H-pyrazole-4-carboxylate

The titled compound was prepared by the reaction of Step 2 intermediate (1.2 g, 6.55 mmol) with 2-bromo-1-(2,6-difluorophenyl)ethanone (1.85 g, 7.85 mmol) using potassium fluoride (570 mg, 9.80 mmol) in dry DMF (12 mL) as per the procedure described in Step 4 of

Intermediate 1 to afford 1.75 g of the product as oil. ¹H NMR (300 MHz, DMSO-d₆): δ 0.82 (t, J=7.2 Hz, 3H), 1.63 (q, J=6.9 Hz, 2H), 2.12 (s, 3H), 3.76 (t, J=7.2 Hz, 2H), 5.19 (s, 2H), 6.28 (br s, 2H), 7.26 (t, J=8.4 Hz, 2H), 7.62-7.68 (m, 1H); ESI (m/z) 338 (M+H)⁺.

### Intermediate 84

2-(2-Chlorophenyl)-2-oxoethyl 5-amino-1-[2-(dimethylamino)ethyl]-3-methyl-1H-pyrazole-4-carboxylate

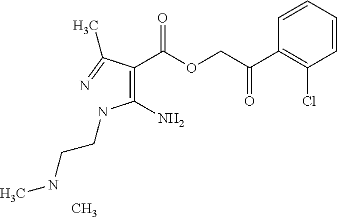

### Step 1: Ethyl 5-amino-I-[2-(dimethylamino)ethyl]-3-methyl-1H-pyrazole-4-carboxylate

The titled compound was prepared by the reaction of ethyl (2E)-2-cyano-3-ethoxybut-2-enoate (7.0 g, 38.20 mmol) with 2-hydrazinyl-N,N-dimethylethanamine (4.73 g, 45.85 mmol) using N, N-diisopropylethylamine (13 mL, 76.41 mmol) in dry ethanol (70 mL) as per the procedure described in Step 2 of Intermediate 1 to yield 7.0 g of the product as oil. ¹H NMR (300 MHz, CDCl₃): δ 1.34 (t, J=6.9 Hz, 3H), 2.31(s, 3H), 2.39 (s, 6H), 2.81 (br s, 2H), 4.11 (br s, 2H), 4.26 (q, J=7.5 Hz, 2H), 6.34 (s, 2H); ESI (m/z) 241 (M+H)⁺.

### Step 2: 5-Amino-1-[2-(dimethylamino)ethyl]-3-methyl-1H-pyrazole-4-carboxylic acid

The titled intermediate was prepared by the ester hydrolysis of Step 1 intermediate (1.2 g, 4.91 mmol) using aqueous solution of potassium hydroxide (2.0 M, 5 mL, 9.82 mmol) as per the procedure described in Step 3 of Intermediate 1 to yield 1.04 g of the product as a liquid. The intermediate was used as such for next Step without any further purification and characterization.

### Step 3: 2-(2-Chlorophenyl)-2-oxoethyl 5-amino-1-[2-(dimethylamino)ethyl]-3-methyl-1H-pyrazole-4-carboxylate

The titled compound was prepared by the reaction of Step 2 intermediate (1.2 g, 5.65 mmol) with 2-bromo-1-(2-chlorophenyl)ethanone (1.3 g, 5.65 mmol) using potassium fluoride (820 mg, 14.12 mmol) in dry DMF (12 mL) as per the procedure described in Step 4 of Intermediate 1 to yield 890 mg of the product as a thick liquid. ¹H NMR (300 MHz, CDCl₃): δ 2.28 (s, 3H), 2.44 (s, 6H), 2.86-2.97 (m, 2H), 4.17 (br s, 2H), 5.32 (s, 2H), 6.46 (br s, 2H), 7.37-7.39 (m, 1H), 7.42-7.48 (m, 2H), 7.64 (d, J=7.8 Hz, 1 H.

### Intermediate 85

2-(2,5-Difluorophenyl)-2-oxoethyl 5-amino-1-ethyl-3-methyl-1H-pyrazole-4-carboxylate

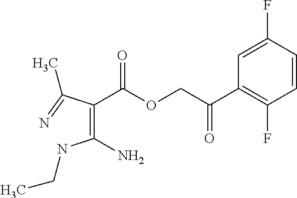 [SMILES: CCN/C(N)=C(\C(C)=N)C(=O)OCC(=O)c1cc(F)ccc1F]

The titled compound was prepared by the reaction of 5-amino-1-ethyl-3-methyl-1H-pyrazole-4-carboxylic acid (700 mg, 4.13 mmol) with 2-bromo-1-(2,5-difluorophenyl)ethanone (972 mg, 4.13 mmol) using potassium fluoride (360 mg, 6.20 mmol) in dry DMF (7.0 mL) as per the procedure described in Step 4 of Intermediate 1 to yield 660 mg of the product as a solid. ¹H NMR (300 MHz, DMSO-d₆): δ 1.20 (t, J=6.9 Hz, 3H), 2.18 (s, 3H), 3.85 (q, J=7.2 Hz, 2H), 5.34 (s, 2H), 6.28 (s, 2H), 7.48-7.54 (m, 1H), 7.61-7.68 (m, 2H); APCI (m/z) 322 (M−H)⁻.

### Intermediate 86

2-(2-Fluoro-4-methoxyphenyl)-2-oxoethyl 5-amino-3-methyl-1-propyl-1H-pyrazole-4-carboxylate

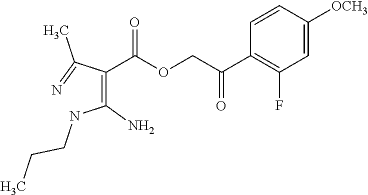 [SMILES: CCCN/C(N)=C(\C(C)=N)C(=O)OCC(=O)c1ccc(OC)cc1F]

The titled compound was prepared by the reaction of 5-amino-3-methyl-1-propyl-1H-pyrazole-4-carboxylic acid (1.0 g, 5.46 mmol) with 2-bromo-1-(2-fluoro-4-methoxyphenyl)ethanone (1.62 g, 6.55 mmol) using potassium fluoride (480 mg, 8.19 mmol) in dry DMF (10 mL) as per the procedure described in Step 4 of Intermediate 1 to yield 1.75 g of the product as a solid. ¹H NMR (300 MHz, DMSO-d₆): δ 0.84 (t, J=7.2 Hz, 3H), 1.65 (q, J=7.2 Hz, 2H), 2.18 (s, 3H), 3.77 (t, J=6.9 Hz, 2H), 3.87 (s, 3H), 5.29 (s, 2H), 6.28 (s, 2H), 6.93-7.05 (m, 2H), 7.86 (t, J=8.7 Hz, 1H). APCI (m/z) 350 (M+H)⁺.

### Intermediate 87

2-(2-Chlorophenyl)-2-oxoethyl 4-amino-1,3-dimethyl-1H-pyrazole-5-carboxylate

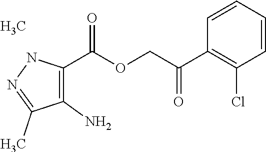

### Step 1: Ethyl 1,3-dimethyl-1H-pyrazole-5-carboxylate

To a stirred solution of ethyl 2,4-dioxovalerate (5.0 g, 31.61 mmol) in dichloromethane (50 mL), methyl hydrazine (85%, 1.45 g, 31.61 mmol) was drop wise added at 0° C. for 1 h. The reaction mixture was stirred at RT for 24 h and refluxed for another 24 h. The mixture was concentrated, diluted with water (50 mL) and extracted with ethyl acetate (250 mL×3). The organic layer was dried over anhydrous sodium sulfate and concentrated under reduced pressure to obtain 5.3 g of the product as oil. The product was used as such for next Step without any purification and characterization.

### Step 2: 1,3-Dimethyl-1H-pyrazole-5-carboxylic acid

A suspension of Step 1 intermediate (5.3 g, 31.52 mmol) in aqueous sodium hydroxide (5%, 32 mL) was refluxed for 4 h. The reaction mixture was cooled to 0° C. and acidified with conc. HCl till pH 2-3. The precipitated solid was filtered and dried well to obtain 3.67 g of the desired product. ¹H NMR (300 MHz, CDCl₃): δ 3.87 (s, 3H), 4.13 (s, 3H), 6.61 (s, 1H), 6.71 (s, 1H).

### Step 3: 1,3-Dimethyl-4-nitro-1H-pyrazole-5-carboxylic acid

To a stirred solution of Step 2 intermediate (3.6 g, 25.68 mmol) in conc. sulfuric acid (26 mL) was added conc. nitric acid (2.3 mL) at -5° C. The mixture was stirred at 0° C. for 15 min and at RT for 30 min. The mixture was heated at 80° C. for 4 h. The mixture was cooled to RT and quenched with ice cold water (30 mL). The precipitated solid was filtered and dried well to obtain 3.2 g of a mixture of the product. ¹H NMR (300 MHz, CDCl₃): δ 2.57 (s, 3H), 4.18 (s, 3H), 7.07 (br s, 1H).

### Step 4: 2-(2-Chlorophenyl)-2-oxoethyl 1,3-dimethyl-4-nitro-1H-pyrazole-5-carboxylate

The titled compound was prepared by the reaction of mixture obtained in Step 3 intermediate (1.2 g, 6.48 mmol) with 2-bromo-1-(2-chlorophenyl)ethanone (1.8 g, 7.77 mmol) using potassium fluoride (564 mg, 9.72 mmol) in dry DMF (12 mL) as per the procedure described in Step 4 of Intermediate 1 to yield 4.19 g of a mixture of the products as a solid. ¹H NMR (300 MHz, CDCl₃): δ 2.52 (s, 3H), 4.08 (s, 3H), 5.57 (s, 2H), 7.40-7.45 (m, 1H), 7.49 (s, 1H), 7.73 (d, J=7.8 Hz, 1H).

### Step 5: 2-(2-Chlorophenyl)-2-oxoethyl 4-amino-1,3-dimethyl-1H-pyrazole-5-carboxylate

To a stirred solution of Step 4 intermediate (2.5 g, 7.40 mmol) in ethyl acetate (25 mL) was added palladium on carbon (10%, 250 mg) and the mixture was stirred under hydrogen atmosphere for 24 h. The mixture was filtered, the filtrate was concentrated and purified by silica gel column chromatography to obtain 569 mg of the titled product as a solid. ¹H NMR (300 MHz, CDCl₃): δ 2.20 (s, 3H), 3.98 (s, 3H), 5.48 (s, 2H), 7.34-7.40 (m, 2H), 7.46 (d, J=3.3 Hz, 3H), 7.68 (d, J=7.5 Hz, 1H); APCI (m/z) 308 (M+H)⁺.

### Intermediate 88

2-(2,6-Difluorophenyl)-2-oxoethyl 5-amino-1-(4-fluorobenzyl)-3-methyl-1H-pyrazole-4-carboxylate

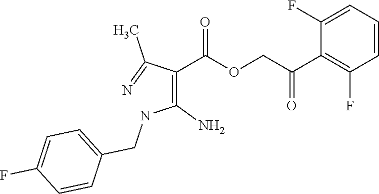 [SMILES: CC(=N)/C(C(=O)OCC(=O)c1c(F)cccc1F)=C(/N)NCc1ccc(F)cc1]

### Step 1: Ethyl 5-amino-1-(4-fluorobenzyl)-3-methyl-1H-pyrazole-4-carboxylate

The titled compound was prepared by the reaction of ethyl (2Z)-2-cyano-3-ethoxybut-2-enoate (1.65 g, 9.01 mmol) with (4-fluorobenzyl)hydrazine (1.5 g, 10.8 mmol) using N, N-diisopropylethylamine (3.1 mL, 18.03 mmol) in dry ethanol (20 mL) as per the procedure described in Step 2 of Intermediate 1 to yield 2.40 g of the product as a solid. ¹H NMR (300 MHz, DMSO-d₆): δ 1.34 (t, J=6.9 Hz, 3H), 2.36 (s, 3H), 4.26 (q, J=6.9 Hz, 2H), 4.89 (s, 2H), 5.06 (s, 2H), 7.00-7.13 (m, 2H), 7.15-7.27 (m, 2H).

### Step 2: 5-Amino-1-(4-fluorobenzyl)-3-methyl-1H-pyrazole-4-carboxylic acid

The titled intermediate was prepared by the ester hydrolysis of Step 1 intermediate (2.35 g, 8.47 mmol) using aqueous solution of potassium hydroxide (2.0 M, 5 mL, 19.94 mmol) and ethanol (15 mL) as per the procedure described in Step 3 of Intermediate 1 to yield 1.5 g of the product as a solid. ¹H NMR (300 MHz, DMSO-d₆): δ 2.12 (s, 3H), 5.04 (s, 2H), 6.29 (s, 2H), 7.08-7.22 (m, 4H), 11.65 (br s, 1H); ESI (m/z) 278 (M+H)⁺.

### Step 3: 2-(2,6-Difluorophenyl)-2-oxoethyl 5-amino-1-(4-fluorobenzyl)-3-methyl-1H-pyrazole-4-carboxylate

The titled compound was prepared by the reaction of Step 2 intermediate (1.0 g, 4.01 mmol) with 2-bromo-1-(2,6-difluorophenyl)ethanone (1.15 g, 4.81 mmol) using potassium fluoride (350 mg, 6.01 mmol) in dry DMF (10 mL) as per the procedure described in Step 4 of Intermediate 1 to afford 1.45 g of the product as oil. ¹H NMR (300 MHz, DMSO-d₆): δ 2.34 (s, 3H), 5.08 (s, 4H), 5.20 (s, 2H), 6.95-7.08 (m, 4H), 7.14-7.27 (m, 2H), 7.43-7.48 (m, 1H); ESI (m/z) 404 (M+H)⁺.

### Intermediate 89

2-(2-Chlorophenyl)-2-oxoethyl 5-amino-3-(difluoromethyl)-1-methyl-1H-pyrazole-4-carboxylate

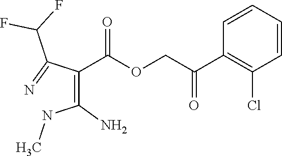 [SMILES: CN/C(N)=C(\C(=N)C(F)F)C(=O)OCC(=O)c1ccccc1Cl]

### Step 1: Sodium (Z)-3-cyano-4-ethoxy-1,1-difluoro-4-oxobut-2-en-2-olate

To a stirred solution of sodium (2.85 g, 124.01 mmol) in ethanol (26 mL) was added ethyl cyanoacetate (12.6 mL, 118.11 mmol) slowly at RT and the reaction mixture was stirred at RT for 1 h. To this mixture methyl difluoroacetate (13.0 g, 118.11 mmol) was added at RT and the reaction mixture was further stirred for 3 h. The reaction mixture was concentrated under reduced pressure and the residue obtained was triturated with hexane (75 mL). The solvent was evaporated under vacuum to yield 25.3 g of the titled product as oil. ¹H NMR (300 MHz, DMSO-d₆): δ 1.14 (t, J=6.9 Hz, 3H), 3.96 (q, J=6.9 Hz, 2H), 6.83 (t, J=55 Hz, 1H).

### Step 2: Ethyl 5-amino-3-(difluoromethyl)-1-methyl-1H-pyrazole-4-carboxylate

To a stirred solution of Step 1 intermediate (25.0 g, 117.30 mmol) in dimethyl carbonate (25 mL), methyl hydrazine sulfate (33.8 g, 234.62 mmol), molecular sieves (25 g) and trifluoroacetic acid (9.0 mL, 117.3 mmol) were added at RT. The reaction mixture was refluxed overnight. The mixture was cooled to RT and filtered. The filtrate was concentrated under reduced pressure to afford 8.1 g of the titled product as a solid. ¹H NMR (300 MHz, CDCl₃): δ 1.23 (t, J=7.2 Hz, 3H), 3.58 (s, 3H), 4.21 (q, J=6.9 Hz, 2H), 6.47 (br s, 2H), 6.96 (t, J=54 Hz, 1H).

### Step 3: 5-Amino-3-(difluoromethyl)-1-methyl-1H-pyrazole-4-carboxylic acid

The titled compound was prepared by the ester hydrolysis of Step 2 intermediate (8.0 g, 36.52 mmol) using aqueous solution of potassium hydroxide (2.0 M, 43 mL, 54.79 mmol) in ethanol (120 mL) as per the procedure described in Step 3 of Intermediate 1 to yield 4.3 g of the product as a solid. ¹H NMR (300 MHz, DMSO-d₆): δ 3.56 (s, 3H), 6.36 (s, 2H), 6.96 (t, J=55 Hz, 1H), 12.40 (s, 1H).

### Step 4: 2-(2-Chlorophenyl)-2-oxoethyl 4-amino-1-(difluoromethyl)-3-methyl-1H-pyrazole-5-carboxylate

The titled compound was prepared by the reaction of Step 3 intermediate (2 g, 10.47 mmol) with 2-bromo-1-(2-chlorophenyl)ethanone (2.4 g, 10.47 mmol) using potassium fluoride (912 mg, 15.70 mmol) in dry DMF (20 mL) as per the procedure described in Step 4 of Intermediate 1 to yield 3.31 g of the product as a solid. ¹H NMR (300 MHz, DMSO-d₆): δ 3.60 (s, 3H), 5.37 (s, 2H), 6.55 (s, 2H), 6.97 (t, J=54 Hz, 1H), 7.48-7.60 (m, 3H), 7.80 (d, J=8.0 Hz, 1H).

### Intermediate 90

2-(2,6-Difluorophenyl)-2-oxoethyl 5-amino-3-(difluoromethyl)-1-methyl-1H-pyrazole-4-carboxylate

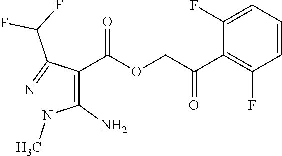 [SMILES: CN/C(N)=C(\C(=N)C(F)F)C(=O)OCC(=O)c1c(F)cccc1F]

The titled compound was prepared by the reaction of 5-amino-3-(difluoromethyl)-1-methyl-1H-pyrazole-4-carboxylic acid (1.2 g, 6.28 mmol) with 2-bromo-1-(2,6-difluorophenyl)ethanone (1.47 g, 6.28 mmol) using potassium fluoride (547 mg, 9.42 mmol) in dry DMF (12 mL) as per the procedure described in Step 4 of Intermediate 1 to afford 1.41 g of the product as a solid. ¹H NMR (300 MHz, DMSO-d₆): δ 3.59 (s, 3H), 5.25 (s, 2H), 6.57 (s, 2H), 6.96 (s, 1H), 7.23-7.30 (m, 2H), 7.63-7.73 (m, 1H); APCI (m/z) 344 (M−H)⁻.

### Intermediate 91

2-(2-Chlorophenyl)-2-oxoethyl 5-amino-3-methyl-1-(2-morpholinoethyl)-1H-pyrazole-4-carboxylate

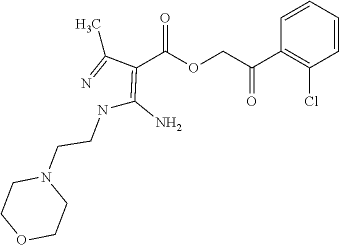 [SMILES: CC(=N)/C(C(=O)OCC(=O)c1ccccc1Cl)=C(/N)NCCN1CCOCC1]

### Step 1: Ethyl 5-amino-3-methyl-1-[2-(morpholin-4-yl)ethyl]-1H-pyrazole-4-carboxylate

The titled compound was prepared by the reaction of ethyl (2E)-2-cyano-3-ethoxybut-2-enoate (18.5 g, 0.10 mmol) with 4-(2-hydrazinylethyl)morpholine (17.58 g, 0.12 mmol) using N,N-diisopropylethylamine (34.8 mL, 0.20 mmol) in dry ethanol (185 mL) as per the procedure described in Step 1 of Intermediate 75 to yield 22.2 g of the product as a liquid. ¹H NMR (300 MHz, DMSO-d₆): δ 1.24 (t, J=7.2 Hz, 3H), 2.14 (s, 3H), 2.40-2.48 (m, 4H), 2.57 (t, J=6.9 Hz, 3H), 3.50-3.63 (m, 4H), 3.92 (t, J=6.3 Hz, 2H), 4.15 (q, J=7.2 Hz, 2H), 6.25 (s, 2H); ESI (m/z) 283 (M+H)⁺.

### Step 2: 5-Amino-3-methyl-1-[2-(morpholin-4-yl)ethyl]-1H-pyrazole-4-carboxylic acid

To a solution of Step 1 intermediate (22 g, 0.07 mmol) in ethanol (155 ml) was added aqueous solution of potassium hydroxide (2.0 M, 77 mL, 0.311 mmol) at RT and mixture was heated to reflux for overnight and additional aqueous solution of potassium hydroxide (2.0 M, 77 mL, 0.311 mmol) was added and continued reaction at reflux temperature for another 24 h. The solvent was evaporated completely and cooled in ice bath and acidified with 2N citric acid (pH 6-7) and extracted with ethyl acetate (3×300 mL) and evaporation solvent to yield 13.1 g of the product as sticky solid. ¹H NMR (300 MHz, DMSO-d₆): δ 2.11 (s, 3H), 2.37-2.48 (m, 4H), 2.55 (t, J=6.3 Hz, 2H), 3.89 (t, J=6.6 Hz, 2H), 6.20 (s, 2H), 11.89 (br s, 1H).

### Step 3: 2-(2-Chlorophenyl)-2-oxoethyl 5-amino-3-methyl-1-(2-morpholinoethyl)-1H-pyrazole-4-carboxylate

The titled compound was prepared by the reaction of Step 2 intermediate (13 g, 0.051 mmol) with 2-bromo-1-(2-chlorophenyl)ethanone (13.14 g, 0.056 mmol) using potassium fluoride (4.46 g, 0.076 mmol) in dry DMF (130 mL) as per the procedure described in Step 4 of Intermediate 1 to yield 15.1 g of the product as thick liquid. ¹H NMR (300 MHz, DMSO-d₆): δ 2.13 (s, 3H), 2.37-2.48 (m, 4H), 2.58 (t, J=6.3 Hz, 2H), 3.48-3.61 (m, 4H), 3.94 (t, J=6.3 Hz, 2H), 5.30 (s, 2H), 6.37 (s, 2H), 7.46-7.54 (m, 1H), 7.58 (d, J=3.9 Hz, 1H), 7.77 (d, J=7.8 Hz, 1H); ESI (m/z) 407 (M+H)⁺.

### Intermediate 92

2-(2,6-Difluorophenyl)-2-oxoethyl 5-amino-1-cyclopropyl-3-methyl-1H-pyrazole-4-carboxylate

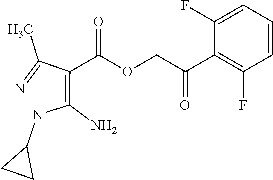 [SMILES: CC(=N)/C(C(=O)OCC(=O)c1c(F)cccc1F)=C(/N)NC1CC1]

### Step 1: Ethyl 5-amino-1-cyclopropyl-3-methyl-1H-pyrazole-4-carboxylate

Cyclopropylhydrazine hydrochloride (3.0 g, 27.32 mmol) was added to a stirred solution of ethyl (2E)-2-cyano-3-ethoxybut-2-enoate (5.0 g, 27.32 mmol) in dry ethanol (50 mL) followed by DIPEA (14 mL, 81.96 mmol) in ethanol (50 mL) at RT. The reaction mixture was heated to 90° C. and stirred overnight at the RT. The ethanol was removed under reduced pressure and the residue obtained was diluted with saturated aqueous sodium bicarbonate solution (50 mL). The aqueous mixture was extracted with ethyl acetate (2×100 mL) and the organic extract was dried over anhydrous sodium sulfate. The solvent was removed under reduced pressure and the compound obtained was purified by silica gel column chromatography to yield 4.0 g of the titled product as oil. ¹H NMR (300 MHz, CDCl₃): δ 1.05-1.09 (m, 4H), 1.33 (t, J=7.5 Hz, 3H), 2.31 (s, 3H), 2.99-3.06 (m, 1H), 4.19-4.30 (m, 2H), 5.27 (br s, 2H); ESI (m/z) 210 (M+H)⁺.

### Step 2: 5-Amino-1-cyclopropyl-3-methyl-1H-pyrazole-4-carboxylic acid

To a stirred solution of Step 1 intermediate (3.9 g, 18.85 mmol) in ethanol (30 mL) was added an aqueous solution of potassium hydroxide (3.0 M, 10 mL, 37.70 mmol) at RT. The mixture was refluxed overnight. The ethanol was removed under reduced pressure and the residue obtained was diluted with water (25 mL). The aqueous mixture was washed with ethyl acetate (2×100 mL). The aqueous layer was acidified with 1N citric acid till pH 3-4. The precipitated solid was collected by filtration to yield 2.0 g of the titled product. ¹H NMR (300 MHz, DMSO-d₆): δ 0.86-0.91 (m, 4H), 2.10 (s, 3H), 3.12-3.17 (m, 1H), 6.12 (s, 2H), 11.71 (s, 1H); ESI (m/z) 182 (M+H)⁺.

### Step 3: 2-(2,6-Difluorophenyl)-2-oxoethyl 5-amino-1-cyclopropyl-3-methyl-1H-pyrazole-4-carboxylate

To a stirred solution of Step 2 intermediate (1.0 g, 5.52 mmol) in dry DMF (10 mL), 2-bromo-1-(2,6-difluorophenyl)ethanone (1.55 g, 6.62 mmol) and potassium fluoride (480 mg, 8.28 mmol) were added at RT. The reaction mixture was stirred overnight at RT. The mixture was basified with saturated aqueous sodium bicarbonate solution till pH 9 and the precipitated solid was filtered. The solid was washed with water (2×10 mL), dried and purified by silica gel column chromatography to yield 1.15 g of the desired product as a solid. ¹H NMR (300 MHz, DMSO-d₆): δ 0.86-0.92 (m, 4H), 2.09 (s, 3H), 3.13-3.18 (m, 1H), 5.19 (s, 2H), 6.29 (s, 2H), 7.26 (t, J=8.7 Hz, 2H), 7.62 (t, J=6.3 Hz, 1H); ESI (m/z) 336 (M+H)⁺.

### Intermediate 93

2-(2,6-Difluorophenyl)-2-oxoethyl 5-amino-3-methyl-1-(2-morpholinoethyl)-1H-pyrazole-4-carboxylate

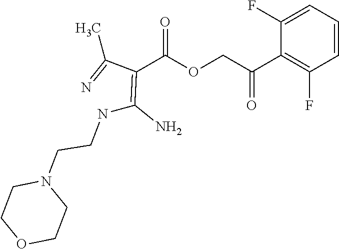 [SMILES: CC(=N)/C(C(=O)OCC(=O)c1c(F)cccc1F)=C(/N)NCCN1CCOCC1]

The titled compound was prepared by the reaction of 5-amino-3-methyl-1-[2-(morpholin-4-yl)ethyl]-1H-pyrazole-4-carboxylic acid (2.0 g, 7.86 mmol) with 2-bromo-1-(2,6-difluorophenyl)ethanone (2.2 g, 9.43 mmol) using potassium fluoride (502 mg, 8.64 mmol) in dry DMF (15 mL) as per the procedure described in Step 4 of Intermediate 1 to afford 1.3 g of the product as a solid. ¹H NMR (300 MHz, CDCl₃): δ 2.28 (s, 3H), 2.79-2.84 (m, 4H), 3.06-3.10 (m, 2H), 3.87-3.90 (m, 4H), 4.31-4.33 (m, 2H), 5.20 (s, 2H), 6.38 (s, 2H), 7.00 (t, J=9.0 Hz, 2H), 7.47 (t, J=6.3 Hz, 2H); ESI (m/z) 409 (M+H)⁺.

### Intermediate 94

2-(2,6-Difluorophenyl)-2-oxoethyl 5-amino-1-[2-(dimethylamino)ethyl]-3-methyl-1H-pyrazole-4-carboxylate

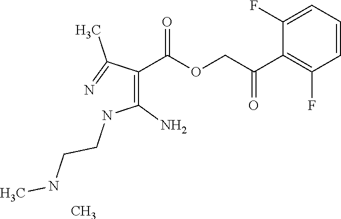

The titled compound was prepared by the reaction of 5-amino-1-[2-(dimethylamino)ethyl]-3-methyl-1H-pyrazole-4-carboxylic acid (1.4 g, 6.65 mmol) with 2-bromo-1-(2,6-difluorophenyl)ethanone (1.8 g, 7.91 mmol) using potassium fluoride (420 mg, 7.25 mmol) in dry DMF (14 mL) as per the procedure described in Step 4 of Intermediate 1 to yield 2.5 g of the product as oil. ¹H NMR (300 MHz, DMSO-d₆): δ 2.12 (s, 3H), 2.17 (s, 3H), 2.50 (s, 3 H, overlapped with DMSO), 2.52 (t, J=7.5 Hz, 2H), 3.90 (t, J=6.3 Hz, 2H), 5.19 (s, 2H), 6.36 (s, 2H), 7.26 (t, J=8.1 Hz, 2H), 7.62-7.71 (m, 1H); APCI (m/z) 367 (M+H)⁺.

### Intermediate 95

2-(2,6-Difluorophenyl)-2-oxoethyl 5-amino-1-(2-methoxyethyl)-3-methyl-1H-pyrazole-4-carboxylate

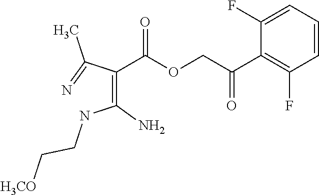 [SMILES: COCCN/C(N)=C(\C(C)=N)C(=O)OCC(=O)c1c(F)cccc1F]

### Step 1: Ethyl 5-amino-1-(2-methoxyethyl)-3-methyl-1H-pyrazole-4-carboxylate

The titled compound was prepared by the reaction of ethyl (2E)-2-cyano-3-ethoxybut-2-enoate (2.0 g, 10.9 mmol) with (2-methoxyethyl)hydrazine (1.08 g, 12.02 mmol) using DIPEA (3.8 mL, 21.85 mmol) in ethanol (20 mL) as per the procedure described in Step 1 of Intermediate 75 to yield 1.35 g of the product as a liquid. ¹H NMR (300 MHz, DMSO-d₆): δ 1.24 (t, J=6.9 Hz, 3H), 2.15 (s, 3H), 3.22 (s, 3H), 3.57 (t, J=5.7 Hz, 2H), 3.97 (t, J=5.7 Hz, 2H), 4.16 (q, J=6.9 Hz, 2H), 6.11 (s, 2H).

### Step 2: 5-Amino-1-(2-methoxyethyl)-3-methyl-1H-pyrazole-4-carboxylic acid

The titled intermediate was prepared by the ester hydrolysis of Step 1 intermediate (1.3 g, 5.78 mmol) using aqueous solution of potassium hydroxide (2 M, 4.0 mL, 11.40 mmol) in ethanol (13 mL) as per the procedure described in Step 3 of Intermediate 1 to yield 690 mg of the product as sticky solid. ¹H NMR (300 MHz, DMSO-d₆): δ 2.08 (s, 3H), 3.22 (s, 3H), 3.57 (t, J=5.4 Hz, 2H), 3.89-3.98 (m, 2H), 6.08 (s, 2H).

### Step 3: 2-(2,6-Difluorophenyl)-2-oxoethyl 5-amino-1-(2-methoxyethyl)-3-methyl-1H-pyrazole-4-carboxylate

The titled compound was prepared by the reaction of Step 2 intermediate (650 mg, 3.26 mmol) with 2-bromo-1-(2,6-difluorophenyl)ethanone (920 mg, 3.91 mmol) using potassium fluoride (284 mg, 4.89 mmol) in dry DMF (6.5 mL) as per the procedure described in Step 4 of Intermediate 1 to yield 260 mg of the product as oil. ¹H NMR (300 MHz, CDCl₃): δ 2.35 (s, 3H), 3.37 (s, 3H), 3.73 (t, J=4.5 Hz, 2H), 4.16 (t, J=4.5 Hz, 2H), 5.22 (s, 2H), 5.74 (br s, 1H), 7.00 (t, J=8.4 Hz, 1H), 7.46-7.49 (m, 1H), 8.01 (s, 1H); APCI (m/z) 354 (M+H)⁺.

### Intermediate 96

2-(2,6-Difluorophenyl)-2-oxoethyl 5-amino-1-methyl-3-(propan-2-yl)-1H-pyrazole-4-carboxylate

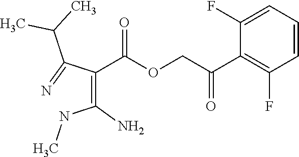 [SMILES: CN/C(N)=C(\C(=N)C(C)C)C(=O)OCC(=O)c1c(F)cccc1F]

### Step 1: 5-Amino-1-methyl-3-(propan-2-yl)-1H-pyrazole-4-carbonitrile

The titled compound was prepared by the reaction of (1-methoxy-2-methylpropylidene)propanedinitrile (2.0 g, 13.3 mmol) with methyl hydrazine sulfate (1.91 g, 13.3 mmol) using DIPEA (4.6 mL, 26.32 mmol) in ethanol (20 mL) as per the procedure described in Step 1 of Intermediate 75 to yield 1.35 g of the product as a solid. ¹H NMR (300 MHz, DMSO-d₆): δ 1.16 (d, J=6.9 Hz, 6H), 2.74-2.82 (m, 1H), 3.44 (s, 3H), 6.43 (s, 2H).

### Step 2: 5-Amino-1-methyl-3-(propan-2-yl)-1H-pyrazole-4-carboxylic acid

To Step 1 intermediate (1.3 g, 7.92 mmol) was added a solution of sodium hydroxide (3.16 g, 79.2 mmol) in water (10 mL) and the mixture was stirred overnight at 100° C. Another same batch of sodium hydroxide was added and the mixture and further stirred for 18 h. The mixture was cooled to RT and diluted with water (10 mL). The aqueous mixture was washed with ethyl acetate (30 mL) andacidified with 1N citric acid till pH 2-3. The aqueous layer was extracted with ethyl acetate (75 mL×2). The organic layer was dried over anhydrous sodium sulfate and concentrated under reduced pressure to obtain 725 mg of the product as sticky solid. ¹H NMR (300 MHz, DMSO-d₆): δ 1.10 (d, J=6.6 Hz, 6H), 3.19-3.35 (m, 1H), 3.43 (s, 3H), 6.07 (s, 2H), 11.70 (br s, 1H).

### Step 3: 2-(2,6-Difluorophenyl)-2-oxoethyl 5-amino-1-methyl-3-(propan-2-yl)-1H-pyrazole-4-carboxylate

The titled compound was prepared by the reaction of Step 2 intermediate (700 mg, 3.82 mmol) with 2-bromo-1-(2,6-difluorophenyl)ethanone (990 mg, 4.20 mmol) using potassium fluoride (335 mg, 5.73 mmol) in dry DMF (7.0 mL) as per the procedure described in Step 4 of Intermediate 1 to yield 625 mg of the product as a solid. ¹H NMR (300 MHz, DMSO-d₆): δ 1.09 (d, J=6.9 Hz, 6H), 3.12-3.20 (m, 1H), 3.47 (s, 3H), 5.19 (s, 2H), 6.26 (s, 2H), 7.25 (t, J=8.4 Hz, 2H), 7.63-7.70 (m, 1H).

### Intermediate 97

2-(2,6-Difluorophenyl)-2-oxoethyl 5-amino-1-(3-methoxypropyl)-3-methyl-1H-pyrazole-4-carboxylate

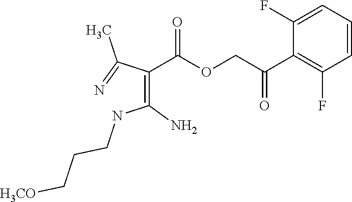 [SMILES: COCCCN/C(N)=C(\C(C)=N)C(=O)OCC(=O)c1c(F)cccc1F]

### Step 1: Ethyl 5-amino-1-(3-methoxypropyl)-3-methyl-1H-pyrazole-4-carboxylate

The titled compound was prepared by the reaction of ethyl (2E)-2-cyano-3-ethoxybut-2-enoate (3.5 g, 19.12 mmol) with (2-methoxypropyl)hydrazine (1.99 g, 19.12 mmol) using DIPEA (6.5 mL, 38.25 mmol) in ethanol (35 mL) as per the procedure described in Step 1 of Intermediate 75 to yield 2.70 g of the product as a liquid. ¹H NMR (300 MHz, CDCl₃): δ 1.35 (t, J=6.9 Hz, 3H), 2.00-2.06 (m, 2H), 2.34 (s, 3H), 3.30 (t, J=5.4 Hz, 2H), 3.35 (s, 3H), 3.96 (t, J=5.7 Hz, 2H), 4.27 (q, J=6.9 Hz, 2H), 5.45 (br s, 2H).

### Step 2: 5-Amino-1-(3-methoxypropyl)-3-methyl-1H-pyrazole-4-carboxylic acid

The titled intermediate was prepared by the ester hydrolysis of Step 1 intermediate (2.70 g, 11.23 mmol) using aqueous solution of potassium hydroxide (2 M, 12.5 mL, 22.38 mmol) in ethanol (27 mL) as per the procedure described in Step 3 of Intermediate 1 to yield 1.85 g of the product as a solid. ¹H NMR (300 MHz, DMSO-d₆): δ 1.84 (t, J=6.9 Hz, 2H), 2.13 (s, 3H), 3.21 (s, 3H), 3.25-3.34 (m, 5H), 3.82 (t, J=6.9 Hz, 2H), 6.09 (s, 2H), 11.75 (s, 1H).

### Step 3: 2-(2,6-Difluorophenyl)-2-oxoethyl 5-amino-1-(3-methoxypropyl)-3-methyl-1H-pyrazole-4-carboxylate

The titled compound was prepared by the reaction of Step 2 intermediate (1.0 g, 4.78 mmol) with 2-bromo-1-(2,6-difluorophenyl)ethanone (1.1 g, 4.78 mmol) using potassium fluoride (410 mg, 7.05 mmol) in dry DMF (10 mL) as per the procedure described in Step 4 of Intermediate 1 to yield 1.18 g of the product as a solid. ¹H NMR (300 MHz, CDCl₃): δ 2.02 (t, J=6.3 Hz, 2H), 2.29 (s, 3H), 3.31 (t, J=5.7 Hz, 2H), 3.36 (s, 3H), 3.94 (t, J=6.3 Hz, 2H), 5.20 (s, 2H), 5.51 (s, 2H), 6.99 (t, J=9.0 Hz, 2H), 7.40-7.50 (m, 1H).

### Intermediate 98

2-(2,6-Difluoro -3-methylphenyl)-2-oxoethyl 5-amino-1-cyclopropyl-3-methyl-1H-pyrazole-4-carboxylate

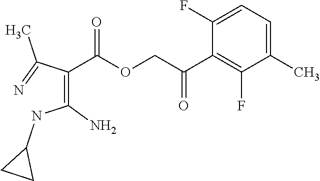 [SMILES: CC(=N)/C(C(=O)OCC(=O)c1c(F)ccc(C)c1F)=C(/N)NC1CC1]

The titled compound was prepared by the reaction of 5-amino-1-cyclopropyl-3-methyl-1H-pyrazole-4-carboxylic acid (500 mg, 2.76 mmol) with 2-bromo-1-(2,6-difluoro-3-methylphenyl)ethanone (826 mg, 3.31 mmol) using potassium fluoride (240 mg, 4.14 mmol) in dry DMF (5.0 mL) as per the procedure described in Step 4 of Intermediate 1 to yield 775 mg of the product as a sticky oil. ¹H NMR (300 MHz, CDCl₃): δ 1.07-1.12 (m, 4H), 2.26 (s, 3H), 2.29 (s, 3H), 2.95-3.07 (m, 1H), 5.19 (s, 2H), 5.38 (s, 2H), 6.88 (t, J=9.9 Hz, 1H), 7.27-7.34 (m, 1H); APCI (m/z) 350 (M+H)⁺.

### Intermediate 99

2-Oxo-2-(2,4,6-trifluorophenyl)ethyl 5-amino-1-cyclopropyl-3-methyl-1H-pyrazole-4-carboxylate

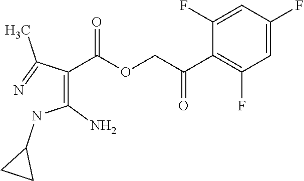 [SMILES: CC(=N)/C(C(=O)OCC(=O)c1c(F)cc(F)cc1F)=C(/N)NC1CC1]

The titled compound was prepared by the reaction of 5-amino-1-cyclopropyl-3-methyl-1H-pyrazole-4-carboxylic acid (600 mg, 3.31 mmol) with 2-bromo-1-(2,4,6-trifluoro-phenyl)ethanone (1.0 g, 3.97 mmol) using potassium fluoride (290 mg, 4.96 mmol) in dry DMF (6.0 mL) as per the procedure described in Step 4 of Intermediate 1 to yield 880 mg of the product as a sticky oil. ¹H NMR (300 MHz, CDCl₃): δ 1.06-1.13 (m, 4H), 2.29 (s, 3H), 3.01-3.07 (m, 1H), 5.16 (s, 2H), 5.36 (s, 2H), 6.76 (t, J=8.4 Hz, 2H).

### Intermediate 100

2-(2,3-Difluorophenyl)-2-oxoethyl 5-amino-1-ethyl-3-methyl-1H-pyrazole-4-carboxylate

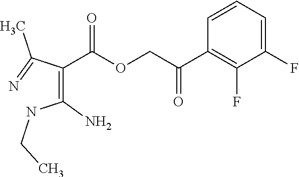 [SMILES: CCN/C(N)=C(\C(C)=N)C(=O)OCC(=O)c1cccc(F)c1F]

The titled compound was prepared by the reaction of 5-amino-1-ethyl-3-methyl-1H-pyrazole-4-carboxylic acid (400 mg, 2.37 mmol) with 2-bromo-1-(2,3-difluorophenyl)ethanone (680 mg, 2.85 mmol) using potassium fluoride (210 mg, 3.55 mmol) in dry DMF (4.0 mL) as per the procedure described in Step 4 of Intermediate 1 to yield 635 mg of the product as a solid. ¹H NMR (300 MHz, CDCl₃): δ 1.38 (t, J=7.2 Hz, 3H), 2.38 (s, 3H), 3.88 (q, J=7.2 Hz, 2H), 5.10 (br s, 2H), 5.37 (s, 2H), 7.23-7.28 (m, 1H), 7.40-7.45 (m, 1H), 7.70-7.78 (m, 1H); ESI (m/z) 324 (M+H)⁺.

### Intermediate 101

2-(2,3-Difluorophenyl)-2-oxoethyl 5-amino-1-cyclopropyl-3-methyl-1H-pyrazole-4-carboxylate

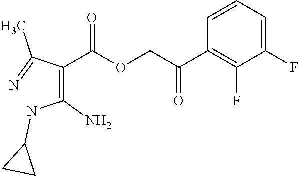 [SMILES: CC(=N)/C(C(=O)OCC(=O)c1cccc(F)c1F)=C(/N)NC1CC1]

The titled compound was prepared by the reaction of 5-amino-1-cyclopropyl-3-methyl-1H-pyrazole-4-carboxylic acid (500 mg, 2.76 mmol) with 2-bromo-1-(2,3-difluorophenyl)ethanone (880 mg, 3.31 mmol) using potassium fluoride (240 mg, 4.14 mmol) in dry DMF (5.0 mL) as per the procedure described in Step 4 of Intermediate 1 to yield 696 mg of the product as oil. ¹H NMR (300 MHz, DMSO-d₆): δ 0.84-0.98 (m, 4H), 2.13 (s, 3H), 3.13-3.20 (m, 1H), 5.35 (s, 2H), 6.28 (s, 2H), 7.33-7.44 (m, 1H), 7.67-7.78 (m, 2H).

### Intermediate 102

2-Oxo-2-(2,4,6-trifluorophenyl)ethyl 5-amino-1-ethyl-3-methyl-1H-pyrazole-4-carboxylate

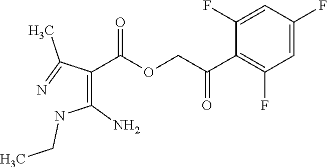 [SMILES: CCN/C(N)=C(\C(C)=N)C(=O)OCC(=O)c1c(F)cc(F)cc1F]

The titled compound was prepared by the reaction of 5-amino-1-ethyl-3-methyl-1H-pyrazole-4-carboxylic acid (500 mg, 2.97 mmol) with 2-bromo-1-(2,4,6-trifluorophenyl)ethanone (910 mg, 3.56 mmol) using potassium fluoride (260 mg, 4.45 mmol) in dry DMF (5.0 mL) as per the procedure described in Step 4 of Intermediate 1 to yield 880 mg of the product as a solid. ¹H NMR (300 MHz, CDCl₃): δ 1.37 (t, J=7.2 Hz, 3H), 2.31 (s, 3H), 3.88 (q, J=7.2 Hz, 2H), 5.14 (s, 2H), 5.16 (s, 2H), 6.75 (t, J=8.4 Hz, 2H). ESI (m/z) 342.26 (M+H)⁺.

### Intermediate 103

2-(2,6-Difluorophenyl)-2-oxoethyl 5-amino-1-cyclopropyl-3-(trifluoromethyl)-1H-pyrazole-4-carboxylate

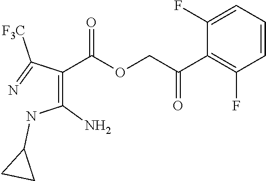 [SMILES: N=C(/C(C(=O)OCC(=O)c1c(F)cccc1F)=C(/N)NC1CC1)C(F)(F)F]

Step 1: Ethyl 5-amino-1-cyclopropyl-3-(trifluoromethyl)-1H-pyrazole-4-carboxylate

The titled compound was prepared by the reaction of ethyl (2E)-3-chloro-2-cyano-4,4,4-trifluorobut-2-enoate (4.5 g, 19.78 mmol) with cyclopropylhydrazine hydrochloride (3.0 g, 27.69 mmol) using triethylamine (5.5 ml, 39.56 mmol) in dry ethanol (45 mL) as per the procedure described in Step 1 of Intermediate 75 to yield 1.62 g of the product as oil. ¹H NMR (300 MHz, CDCl₃): δ 1.09-1.18 (m, 4H), 1.31 (t, J=6.6 Hz, 3H), 3.11-3.17 (m, 1H), 4.28 (q, J=6.6 Hz, 2H), 5.46 (br s, 2H).

### Step 2: 5-Amino-1-cyclopropyl-3-(trifluoromethyl)-1H-pyrazole-4-carboxylic acid

The titled intermediate was prepared by the ester hydrolysis of Step 1 intermediate (1.67 g, 6.34 mmol) using aqueous solution of potassium hydroxide (2.0 M, 4.2 mL, 12.68 mmol) in ethanol (27 mL) as per the procedure described in Step 3 of Intermediate 1 to yield 1.18 g of the product as a solid. ¹H NMR (300 MHz, DMSO-d₆): δ 0.95-1.03 (m, 4H), 3.33-3.36 (m, 1H), 6.57 (s, 2H), 12.42 (br s, 1H).

### Step 3: 2-(2,6-Difluorophenyl)-2-oxoethyl 5-amino-1-cyclopropyl-3-(trifluoromethyl)-1H-pyrazole-4-carboxylate

The titled compound was prepared by the reaction of Step 2 intermediate (1.15 g, 4.89 mmol) with 2-bromo-1-(2,6-difluorophenyl)ethanone (1.38 g, 5.86 mmol) using potassium fluoride (426 mg, 7.33 mmol) in dry DMF (10 mL) as per the procedure described in Step 4 of Intermediate 1 to yield 1.34 g of the product as a solid. ¹H NMR (300 MHz, DMSO-d₆): δ 0.93-1.04 (m, 4H), 3.29-3.35 (m, 1H), 5.24 (s, 2H), 6.76 (s, 2H), 7.24 (t, J=8.7 Hz, 2H), 7.61-7.73 (m, 1H).

### Intermediate 104

2-(2,6-Difluorophenyl)-2-oxoethyl 5-amino-3-(difluoromethyl)-1-ethyl-1H-pyrazole-4-carboxylate

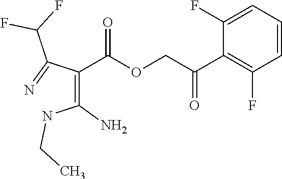 [SMILES: CCN/C(N)=C(\C(=N)C(F)F)C(=O)OCC(=O)c1c(F)cccc1F]

### Step 1: Ethyl (2E)-3-chloro-2-cyano-4,4-difluorobut-2-enoate

The titled compound was prepared by the reaction of ethyl cyanoacetate (5.0 g, 44.25 mmol) with ethyl difluoroacetate (5.7 mL, 54.31 mmol) using sodium metal (1.0 g, 44.25 mmol) in dry ethanol (25 mL) as per the procedure described in Step 1 of Intermediate 39 followed by treating with phosphorus pentachloride (9.2 g, 44.25 mmol) in dichloromethane (50 mL) to give 3.2 g of the desired product as oil. ¹H NMR (300 MHz, CDCl₃): δ 1.39 (t, J=7.2 Hz, 3H), 4.38 (q, J=7.2 Hz, 2H), 6.68 (t, J=53 Hz, 1H).

### Step 2: Ethyl 5-amino-3-(difluoromethyl)-1-ethyl-1H-pyrazole-4-carboxylate

The titled compound was prepared by the reaction of Step 1 intermediate (2.4 g, 11.45 mmol) with ethyl hydrazine oxalate (1.7 g, 11.45 mmol) using triethylamine (3.2 mL, 22.91 mmol) in dry ethanol (25 mL) as per the procedure described in Step 1 of Intermediate 75 to yield 1.5 g of the product as oil. ¹H NMR (300 MHz, DMSO-d₆): δ 1.24 (t, J=6.6 Hz, 6H), 3.97 (q, J=7.5 Hz, 2H), 4.18 (q, J=6.9 Hz, 2H), 6.43 (s, 2H), 6.94 (t, J=54 Hz, 1H).

### Step 3: 4-Amino-1-(difluoromethyl)-3-ethyl-1H-pyrazole-5-carboxylic acid

The titled intermediate was prepared by the ester hydrolysis of Step 2 intermediate (1.67 g, 6.84 mmol) using aqueous solution of potassium hydroxide (2 M, 5.0 mL, 13.71 mmol) in ethanol (10 mL) as per the procedure described in Step 3 of Intermediate 1 to yield 660 mg of the product as a solid. ¹H NMR (300 MHz, DMSO-d₆): δ 1.22 (t, J=6.9 Hz, 3H), 3.96 (q, J=6.9 Hz, 2H), 6.40 (s, 2H), 6.97 (t, J=54.3 Hz, 1H), 12.31 (br s, 1H).

### Step 4: 2-(2,6-Difluorophenyl)-2-oxoethyl 5-amino-3-(difluoromethyl)-1-ethyl-1H-pyrazole-4-carboxylate

The titled compound was prepared by the reaction of Step 3 intermediate (650 mg, 3.16 mmol) with 2-bromo-1-(2,6-difluorophenyl)ethanone (745 mg, 3.16 mmol) using potassium fluoride (275 mg, 4.74 mmol) in dry DMF (7.0 mL) as per the procedure described in Step 4 of Intermediate 1 to yield 750 mg of the product as a solid. ¹H NMR (300 MHz, DMSO-d₆): δ 1.24 (t, J=6.6 Hz, 3H), 3.99 (q, J=7.2 Hz, 2H), 5.25 (s, 2H), 6.59 (s, 2H), 6.78-7.14 (m, 1H), 6.96 (t, J=54 Hz, 1H), 7.26 (t, J=8.7 Hz, 2H), 7.63-7.70 (m, 1H).

### Intermediate 105

2-(2,6-Difluorophenyl)-2-oxoethyl 5-amino-1-ethyl-3-(trifluoromethyl)-1H-pyrazole-4-carboxylate

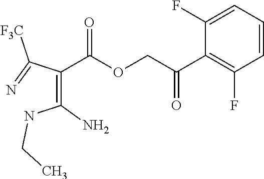 [SMILES: CCN/C(N)=C(\C(=N)C(F)(F)F)C(=O)OCC(=O)c1c(F)cccc1F]

### Step 1: Ethyl 5-amino-1-ethyl-3-(trifluoromethyl)-1H-pyrazole-4-carboxylate

The titled compound was prepared by the reaction of ethyl (2E)-3-chloro-2-cyano-4,4,4-trifluorobut-2-enoate (3.0 g, 13.18 mmol) with ethyl hydrazine oxalate (1.97 g, 13.18 mmol) using triethylamine (3.8 mL, 26.37 mmol) in dry ethanol (30 mL) as per the procedure described in Step 1 of Intermediate 75 to yield 900 mg of the product as a solid. ¹H NMR (300 MHz, DMSO-d₆): δ 1.23 (t, J=7.2 Hz, 6H), 4.00 (q, J=7.2 Hz, 2H), 4.19 (q, J=6.9 Hz, 2H), 6.60 (s, 2H).

### Step 2: 5-Amino-1-ethyl-3-(trifluoromethyl)-1H-pyrazole-4-carboxylic acid

The titled intermediate was prepared by the ester hydrolysis of Step 1 intermediate (900 mg, 3.58 mmol) using aqueous solution of potassium hydroxide (2 M, 3.0 mL, 7.16 mmol) in ethanol (9.0 mL) as per the procedure described in Step 3 of Intermediate 1 to yield 565 mg of the product as a solid. ¹H NMR (300 MHz, DMSO-d₆): δ 1.23 (t, J=6.9 Hz, 3H), 3.98 (q, J=6.9 Hz, 2H), 6.57 (s, 2H), 12.38 (br s, 1H).

### Step 3: 2-(2,6-Difluorophenyl)-2-oxoethyl 5-amino-1-ethyl-3-(trifluoromethyl)-1H-pyrazole-4-carboxylate

The titled compound was prepared by the reaction of Step 2 intermediate (550 mg, 2.46 mmol) with 2-bromo-1-(2,6-difluorophenyl)ethanone (695 mg, 2.95 mmol) using potassium fluoride (214 mg, 3.69 mmol) in dry DMF (5.0 mL) as per the procedure described in Step 4 of Intermediate 1 to yield 275 mg of the product as a solid. ¹H NMR (300 MHz, DMSO-d₆): δ 1.24 (t, J=6.9 Hz, 3H), 4.01 (q, J=6.9 Hz, 2H), 5.25 (s, 2H), 6.76 (s, 2H), 7.26 (t, J=8.7 Hz, 2H), 7.60-7.74 (m, 1H).

### Intermediate 106

2-(2,6-Difluorophenyl)-2-oxoethyl 5-amino-1-ethyl-1H-imidazole-4-carboxylate

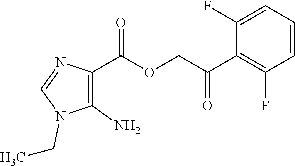 [SMILES: CCn1cnc(C(=O)OCC(=O)c2c(F)cccc2F)c1N]

### Step 1: Ethyl 5-amino-1-ethyl-1H-imidazole-4-carboxylate

The titled compound was prepared by the reaction of ethyl amino(cyano)acetate (3.6 g, 28.09 mmol) with triethylorthoformate (4.7 mL, 28.09 mmol) using ethylamine (2 M in Me0 H, 14.5 mL, 28.09 mmol) in methyl cyanide (70 mL) as per the procedure described in Step 1 of Intermediate 7 to yield 1.15 g of the product as a solid. ¹H NMR (300 MHz, DMSO-d₆): δ 1.22 (t, J=6.9 Hz, 6H), 3.82 (q, J=6.9 Hz, 2H), 4.14 (q, J=6.9 Hz, 2H), 6.00 (s, 2H), 7.15 (s, 1H).

### Step 2: 5-Amino-1-ethyl-1H-imidazole-4-carboxylic acid

The titled intermediate was prepared by the ester hydrolysis of Step 1 intermediate (1.1 g, 6.00 mmol) using aqueous solution of potassium hydroxide (2 M, 3.0 mL, 9.00 mmol) in ethanol (6.0 mL) as per the procedure described in Step 3 of Intermediate 1 to yield 950 mg of the product as a solid. The product was used as such for next Step without characterization.

### Step 3: 2-(2,6-Difluorophenyl)-2-oxoethyl 5-amino-1-ethyl-1H-imidazole-4-carboxylate

The titled compound was prepared by the reaction of Step 2 intermediate (930 mg, 4.79 mmol) with 2-bromo-1-(2,6-difluorophenyl)ethanone (1.35 g, 5.75 mmol) using potassium fluoride (420 mg, 7.18 mmol) in dry DMF (9.0 mL) as per the procedure described in Step 4 of Intermediate 1 to yield 920 mg of the product as a solid. ¹H NMR (300 MHz, DMSO-d₆): δ 1.23 (t, J=6.9 Hz, 3H), 3.82 (q, J=7.2 Hz, 2H), 5.18 (s, 2H), 6.14 (s, 2H), 7.17-7.30 (m, 3H), 7.60-7.78 (m, 1H).

### Intermediate 107

2-(2,6-Difluorophenyl)-2-oxoethyl 5-amino-3-(methoxymethyl)-1-methyl-1H-pyrazole-4-carboxylate

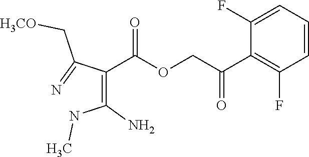 [SMILES: CN/C(N)=C(\C(=N)COC)C(=O)OCC(=O)c1c(F)cccc1F]

### Step 1: 5-Amino-3-(methoxymethyl)-1-methyl-1H-pyrazole-4-carbonitrile

The titled compound was prepared by the reaction of (1,2-dimethoxyethylidene)propanedinitrile (1.95 g, 12.85 mmol) with methyl hydrazine sulfate (1.84 g, 12.18 mmol) using DIPEA (4.4 mL, 25.6 mmol) in ethanol (20 mL) as per the procedure described in Step 1 of Intermediate 75 to yield 980 mg of the product as a solid. ¹H NMR (300 MHz, CDCl₃): δ 3.43 (s, 3H), 3.63 (s, 3H), 4.40 (s, 2H).

### Step 2: 5-Amino-3-(methoxymethyl)-1-methyl-1H-pyrazole-4-carboxylic acid

A suspension of Step 1 intermediate (980 mg, 5.89 mmol) and sodium hydroxide (3.0 g, 75.0 mmol) in water (10 mL) was heated at 90° C. for 72 h. The mixture was cooled to RT and acidified with 1N citric acid till pH 2-3. The aqueous layer was extracted with ethyl acetate (75 mL×2) and the organic layer was dried over anhydrous sodium sulfate. The solution was concentrated under reduced pressure to obtain 450 mg of the titled product as a solid. ¹H NMR (300 MHz, DMSO-d₆): δ 3.21 (s, 3H), 3.50 (s, 3H), 4.34 (s, 2H), 6.17 (s, 2H), 11.84 (br s, 1H).

### Step 3: 2-(2,6-Difluorophenyl)-2-oxoethyl 5-amino-3-(methoxymethyl)-1-methyl-1H-pyrazole-4-carboxylate

The titled compound was prepared by the reaction of Step 2 intermediate (430 mg, 2.32 mmol) with 2-bromo-1-(2,6-difluorophenyl)ethanone (660 mg, 2.78 mmol) using potassium fluoride (210 mg, 3.48 mmol) in dry DMF (4.5 mL) as per the procedure described in Step 4 of Intermediate 1 to yield 380 mg of the product as sticky solid. ¹H NMR (300 MHz, CDCl₃): δ 3.43 (s, 3H), 3.60 (s, 3H), 4.55 (s, 2H), 5.10 (br s, 2H), 5.21 (s, 2H), 6.99 (t, J=10.5 Hz, 2H), 7.44-7.47 (m, 1H).

### Intermediate 108

2-(2,6-Difluorophenyl)-2-oxoethyl 5-amino-1,3-diethyl-1H-pyrazole-4-carboxylate

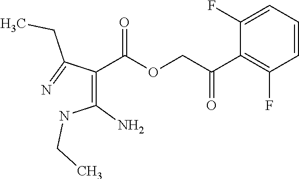 [SMILES: CCN/C(N)=C(\C(=N)CC)C(=O)OCC(=O)c1c(F)cccc1F]

### Step 1: Ethyl 5-amino-1,3-diethyl-1H-pyrazole-4-carboxylate

The titled compound was prepared by the reaction of ethyl (2Z)-2-cyano-3-ethoxypent-2-enoate (10 g, 50.74 mmol) with ethyl hydrazine oxalate (7.61 g, 50.78 mmol) using triethylamine (14.5 mL, 101.40 mmol) in ethanol (100 mL) as per the procedure described in Step 1 of Intermediate 75 to yield 7.4 g of the product as oil. ¹H NMR (300 MHz, CDCl₃): δ 1.23 (t, J=7.2 Hz, 3H), 1.25-1.40 (m, 6H), 2.74 (q, J=7.2 Hz, 2H), 3.89 (q, J=7.5 Hz, 2H), 4.26 (q, J=7.5 Hz, 2H), 5.09 (s, 2H).

### Step 2: 5-Amino-1,3-diethyl-1H-pyrazole-4-carboxylic acid

The titled intermediate was prepared by the ester hydrolysis of Step 1 intermediate (7.3 g, 34.6 mmol) using aqueous solution of potassium hydroxide (2 M, 20 mL, 69.19 mmol) in ethanol (40 mL) as per the procedure described in Step 3 of Intermediate 1 to yield 2.8 g of the product as a solid. ¹H NMR (300 MHz, DMSO-d₆): δ 1.03-1.22 (m, 6H), 2.58 (q, J=7.8 Hz, 2H), 3.34-3.89 (m, 2H), 6.12 (s, 2H), 11.69 (br s, 1H).

### Step 3: 2-(2,6-Difluorophenyl)-2-oxoethyl 5-amino-1,3-diethyl-1H-pyrazole-4-carboxylate

The titled compound was prepared by the reaction of Step 2 intermediate (750 mg, 4.09 mmol) with 2-bromo-1-(2,6-difluorophenyl)ethanone (1.15 g, 4.91 mmol) using potassium fluoride (360 mg, 6.14 mmol) in dry DMF (7.5 mL) as per the procedure described in Step 4 of Intermediate 1 to yield 820 mg of the product as a solid. ¹H NMR (300 MHz, CDCl₃): δ 1.19 (t, J=7.5 Hz, 3H), 1.36 (t, J=7.5 Hz, 3H), 2.72 (q, J=7.2 Hz, 2H), 3.89 (q, J=7.2 Hz, 2H), 5.16 (s, 2H), 5.20 (s, 2H), 6.98 (t, J=8.4 Hz, 2H), 7.40-7.50 (m, 1H).

### Intermediate 109

2-[2-Fluoro-3-(trifluoromethyl)phenyl]-2-oxoethyl 5-amino-1-ethyl-3-methyl-1H-pyrazole-4-carboxylate

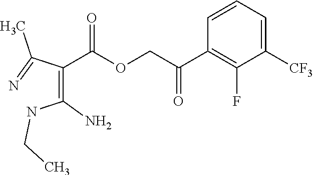 [SMILES: CCN/C(N)=C(\C(C)=N)C(=O)OCC(=O)c1cccc(C(F)(F)F)c1F]

The titled compound was prepared by the reaction of 5-amino-1-ethyl-3-methyl-1H-pyrazole-4-carboxylic acid (500 mg, 2.95 mmol) with 2-bromo-1-[2-fluoro-3-(trifluoromethyl)phenyl]ethanone (1.0 g, 3.54 mmol) using potassium fluoride (257 mg, 4.43 mmol) in dry DMF (5.0 mL) as per the procedure described in Step 4 of Intermediate 1 to yield 745 mg of the product as a solid. ¹H NMR (300 MHz, DMSO-d₆): δ 1.20 (t, J=6.9 Hz, 3H), 2.18 (s, 3H), 3.85 (q, J=6.6 Hz, 2H), 5.37 (s, 2H), 6.29 (s, 2H), 7.58 (t, J=7.8 Hz, 1H), 8.09 (t, J=6.9 Hz, 1H), 8.19 (t, J=6.9 Hz, 1H).

### Intermediate 110

2-(2,6-Difluorophenyl)-2-oxoethyl 5-amino-1-ethyl-1H-pyrazole-4-carboxylate

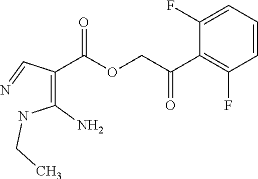 [SMILES: CCN/C(N)=C(\C=N)C(=O)OCC(=O)c1c(F)cccc1F]

### Step 1: Ethyl 5-amino-1-ethyl-1H-pyrazole-4-carboxylate

The titled compound was prepared by the reaction of ethyl (2Z)-2-cyano-3-(dimethylamino)prop-2-enoate (2.0 g, 11.85 mmol) with ethyl hydrazine oxalate (2.14 g, 14.26 mmol) using N,N′-diisopropylethylamine (4.0 mL, 23.78 mmol) in dry ethanol (20 mL) as per the procedure described in Step 1 of Intermediate 75 to yield 2.1 g of the product as a solid. ¹H NMR (300 MHz, CDCl₃): δ 1.25-1.49 (m, 6H), 3.94 (q, J=7.2 Hz, 2H), 4.17-4.30 (m, 2H), 5.04 (br s, 2H), 7.62 (s, 1H).

### Step 2: 5-Amino-1-ethyl-1H-pyrazole-4-carboxylic acid

The titled intermediate was prepared by the ester hydrolysis of Step 1 intermediate (2.0 g, 1.18 mmol) using aqueous solution of potassium hydroxide (2.0 M, 7.0 mL, 23.66 mmol) in ethanol (20 mL) as per the procedure described in Step 3 of Intermediate 1 to yield 820 mg of the product as a solid. ¹H NMR (300 MHz, DMSO-d₆): δ 1.20 (t, J=7.2 Hz, 3H), 3.89 (q, J=7.2 Hz, 2H), 6.15 (s, 2H), 7.40 (s, 1H), 11.67 (br s, 1H).

### Step 3: 2-(2,6-Difluorophenyl)-2-oxoethyl 5-amino-1-ethyl-1H-pyrazole-4-carboxylate

The titled compound was prepared by the reaction of Step 2 intermediate (500 mg, 3.54 mmol) with 2-bromo-1-(2,6-difluorophenyl)ethanone (1.6 g, 4.25 mmol) using potassium fluoride (500 mg, 5.31 mmol) in dry DMF (5.0 mL) as per the procedure described in Step 4 of Intermediate 1 to yield 810 mg of the product as a solid. ¹H NMR (300 MHz, CDCl₃): δ 1.41 (t, J=7.2 Hz, 3H), 3.97 (q, J=7.2 Hz, 2H), 5.19 (s, 2H), 7.00 (t, J=8.1 Hz, 2H), 7.40-7.50 (m, 1H), 7.68 (s, 1H).

### Intermediate 111

2-(2,6-Difluorophenyl)-2-oxoethyl 5-amino-1-tert-butyl-3-methyl-1H-pyrazole-4-carboxylate

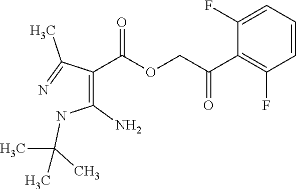 [SMILES: CC(=N)/C(C(=O)OCC(=O)c1c(F)cccc1F)=C(/N)NC(C)(C)C]

### Step 1: Ethyl 5-amino-1-tert-butyl-3-methyl-1H-pyrazole-4-carboxylate

The titled compound was prepared by the reaction of ethyl (2E)-2-cyano-3-ethoxybut-2-enoate (5.0 g, 27.32 mmol) with tert-butyl hydrazine hydrochloride (3.45 g, 27.32 mmol) using N,N′-diisopropylethylamine (14 mL, 81.96 mmol) in ethanol (50 mL) as per the procedure described in Step 1 of Intermediate 75 to yield 5.56 g of the product as a solid. ¹H NMR (300 MHz, CDCl₃): δ 1.13 (s, 9H), 1.30-1.40 (m, 3H), 2.38 (s, 3H), 4.20 (q, J=7.2 Hz, 2H).

### Step 2: 5-Amino-1-tert-butyl-3-methyl-1H-pyrazole-4-carboxylic acid

The titled intermediate was prepared by the ester hydrolysis of Step 1 intermediate (5.5 g, 24.41 mmol) using aqueous solution of potassium hydroxide (2.0 M, 18 mL, 36.61 mmol) in ethanol (60 mL) as per the procedure described in Step 3 of Intermediate 1 to yield 2.07 g of the product as a white solid. ¹H NMR (300 MHz, DMSO-d₆): δ 1.49 (s, 9H), 2.12 (s, 3H), 6.05 (s, 2H), 11.76 (s, 1H).

### Step 3: 2-(2,6-Difluorophenyl)-2-oxoethyl 5-amino-1-tert-butyl-3-methyl-1H-pyrazole-4-carboxylate

The titled compound was prepared by the reaction of Step 2 intermediate (500 mg, 2.53 mmol) with 2-bromo-1-(2,6-difluorophenyl)ethanone (715 mg, 3.04 mmol) using potassium fluoride (220 mg, 3.80 mmol) in dry DMF (5.0 mL) as per the procedure described in Step 4 of Intermediate 1 to yield 695 mg of the product as a solid. ¹H NMR (300 MHz, CDCl₃): δ 1.60 (s, 9H), 2.26 (s, 3H), 5.18 (s, 2H), 5.35 (br s, 2H), 6.98 (t, J=8.4 Hz, 2H), 7.42-7.50 (m, 1H).

### Intermediate 112

2-(2-Fluorophenyl)-2-oxoethyl 5-amino-1-ethyl-3-methyl-1H-pyrazole-4-carboxylate

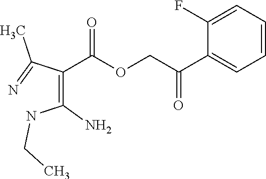 [SMILES: CCN/C(N)=C(\C(C)=N)C(=O)OCC(=O)c1ccccc1F]

The titled compound was prepared by the reaction of 5-amino-1-ethyl-3-methyl-1H-pyrazole-4-carboxylic acid (500 mg, 2.95 mmol) with 2-bromo-142-fluoro-phenyllethanone (770 mg, 3.54 mmol) using potassium fluoride (260 mg, 4.43 mmol) in dry DMF (5.0 mL) as per the procedure described in Step 4 of Intermediate 1 to yield 510 mg of the product as a solid. ¹H NMR (300 MHz, DMSO-d₆): δ 1.20 (t, J=7.2 Hz, 3H), 2.18 (s, 3H), 3.85 (q, J=7.2 Hz, 2H), 5.34 (s, 2H), 6.28 (s, 2H), 7.37-7.46 (m, 2H), 7.69-7.74 (m, 1H), 7.91 (t, J=7.2 Hz, 1H).

### Intermediate 113

2-(2,6-Difluorophenyl)-2-oxoethyl 5-amino-3-methyl-1-(2-methylpropyl)-1H-pyrazole-4-carboxylate

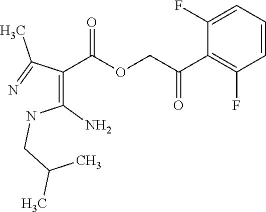 [SMILES: CC(=N)/C(C(=O)OCC(=O)c1c(F)cccc1F)=C(/N)NCC(C)C]

### Step 1: Ethyl 5-amino-3-methyl-1-(2-methylpropyl)-1H-pyrazole-4-carboxylate

The titled compound was prepared by the reaction of ethyl (2E)-2-cyano-3-ethoxybut-2-enoate (2.00 g, 10.92 mmol) with (2-methylpropyl)hydrazine (1.15 g, 13.11 mmol) using triethylamine (3.0 mL, 21.85 mmol) in ethanol (20 mL) as per the procedure described in Step 1 of Intermediate 75 to yield 1.17 g of the product as a solid. ¹H NMR (300 MHz, CDCl₃): δ 0.94 (d, J=6.6 Hz, 6H), 1.34 (t, J=6.9 Hz, 3H), 2.15-2.25 (m, 1H), 2.35 (s, 3H), 3.63 (d, J=7.2 Hz, 2H), 4.27 (q, J=6.9 Hz, 2H), 5.10 (br s, 1H).

### Step 2: 5-Amino-3-methyl-1-(2-methylpropyl)-1H-pyrazole-4-carboxylic acid

The titled intermediate was prepared by the ester hydrolysis of Step 1 intermediate (1.1 g, 4.88 mmol) using aqueous solution of potassium hydroxide (2.0 M, 4.0 mL, 7.32 mmol) in ethanol (11 mL) as per the procedure described in Step 3 of Intermediate 1 to yield 336 mg of the product as a solid. ¹H NMR (300 MHz, DMSO-d₆): δ 0.82 (d, J=6.9 Hz, 6H), 2.00-2.10 (m, 1H), 2.13 (s, 3H), 3.59 (d, J=6.9 Hz, 2H), 6.12 (s, 2H), 11.71 (br s, 1H).

### Step 3: 2-(2,6-Difluorophenyl)-2-oxoethyl 5-amino-3-methyl-1-(2-methylpropyl)-1H-pyrazole-4-carboxylate

The titled compound was prepared by the reaction of Step 2 intermediate (330 mg, 1.67 mmol) with 2-bromo-1-(2,6-difluorophenyl)ethanone (472 mg, 2.00 mmol) using potassium fluoride (145 mg, 2.51 mmol) in dry DMF (3.5 mL) as per the procedure described in Step 4 of Intermediate 1 to yield 275 mg of the product as a solid. ¹H NMR (300 MHz, DMSO-d₆): δ 0.82 (d, J=6.9 Hz, 6H), 1.95-2.10 (m, 1H), 2.12 (s, 3H), 3.61 (d, J=6.9 Hz, 2H), 5.18 (s, 2H), 6.27 (s, 2H), 7.25 (t, J=8.7 Hz, 2H), 7.60-7.70 (m, 1H).

### Intermediate 114

2-(2-Chlorophenyl)-2-oxoethyl 5-amino-1-ethyl-3-(trifluoromethyl)-1H-pyrazole-4-carboxylate

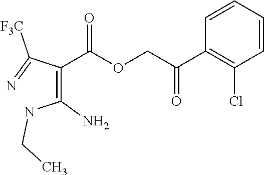 [SMILES: CCN/C(N)=C(\C(=N)C(F)(F)F)C(=O)OCC(=O)c1ccccc1Cl]

The titled compound was prepared by the reaction of 5-amino-1-ethyl-3-(trifluoromethyl)-1H-pyrazole-4-carboxylic acid (600 mg, 2.68 mmol) with 2-bromo-1-(2-chlorophenyl) ethanone (755 mg, 3.22 mmol) using potassium fluoride (235 mg, 4.03 mmol) in dry DMF (6.0 mL) as per the procedure described in Step 4 of Intermediate 1 to yield 653 mg of the product as a solid. ¹H NMR (300 MHz, DMSO-d₆): δ 1.25 (t, J=7.2 Hz, 3H), 4.02 (q, J=7.5 Hz, 2H), 5.38 (s, 2H), 6.75 (s, 2H), 7.45-7.54 (m, 1H), 7.58 (s, 2H), 7.80 (d, J=7.8 Hz, 1H).

### Intermediate 115

2-(2-Methoxyphenyl)-2-oxoethyl 5-amino-1-ethyl-3-methyl-1H-pyrazole-4-carboxylate

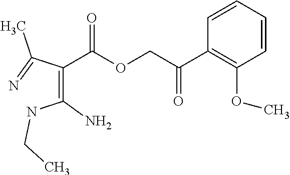 [SMILES: CCN/C(N)=C(\C(C)=N)C(=O)OCC(=O)c1ccccc1OC]

The titled compound was prepared by the reaction of 5-amino-1-ethyl-3-methyl-1H-pyrazole-4-carboxylic acid (500 mg, 2.95 mmol) with 2-bromo-1-(2-methoxyphenyl)ethanone (812 mg, 3.54 mmol) using potassium fluoride (257 mg, 4.43 mmol) in dry DMF (5.0 mL) as per the procedure described in Step 4 of Intermediate 1 to yield 656 mg of the product as a solid. ¹H NMR (300 MHz, DMSO-d₆): δ 1.20 (t, J=7.2 Hz, 3H), 2.17 (s, 3H), 3.85 (q, J=7.2 Hz, 2H), 3.94 (s, 3H), 5.28 (s, 2H), 6.26 (s, 2H), 7.09 (t, J=7.8 Hz, 1H), 7.24 (d, J=8.4 Hz, 1H), 7.63 (t, J=7.5 Hz, 1H), 7.75 (d, J=7.5 Hz, 1H).

### Intermediate 116

2-(2,6-Difluorophenyl)-2-oxoethyl 5-amino-1-(2-methylpropyl)-3-(trifluoromethyl)-1H-pyrazole-4-carboxylate

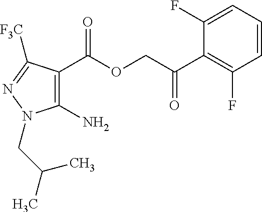 [SMILES: CC(C)Cn1nc(C(F)(F)F)c(C(=O)OCC(=O)c2c(F)cccc2F)c1N]

### Step 1: Ethyl 5-amino-1-(2-methylpropyl)-3-(trifluoromethyl)-1H-pyrazole-4-carboxylate

The titled compound was prepared by the reaction of ethyl (2E)-3-chloro-2-cyano-4,4,4-trifluorobut-2-enoate (6.45 g, 28.36 mmol) with (2-methylpropyl)hydrazine (2.5 g, 28.36 mmol) using triethylamine (8.0 mL, 56.72 mmol) in ethanol (60 mL) as per the procedure described in Step 1 of Intermediate 75 to yield 1.31 g of the product as a solid. ¹H NMR (300 MHz, CDCl₃): δ 0.94 (d, J=6.9 Hz, 6H), 1.33 (t, J=6.9 Hz, 3H), 2.15-2.34 (m, 1H), 3.70 (d, J=7.2 Hz, 2H), 4.26 (q, J=6.9 Hz, 2H).

### Step 2: 5-Amino-1-(2-methylpropyl)-3-(trifluoromethyl)-1H-pyrazole-4-carboxylic acid

The titled intermediate was prepared by the ester hydrolysis of Step 1 intermediate (1.4 g, 5.01 mmol) using aqueous solution of potassium hydroxide (2 M, 5.0 mL, 7.51 mmol) in ethanol (15 mL) as per the procedure described in Step 3 of Intermediate 1 to yield 730 mg of the product as a solid. ¹H NMR (300 MHz, DMSO-d₆): δ 0.84 (d, J=6.3 Hz, 6H), 2.00-2.09 (m, 1H), 3.79 (d, J=7.2 Hz, 2H), 6.58 (s, 2H), 12.40 (br s, 1H).

### Step 3: 2-(2,6-Difluorophenyl)-2-oxoethyl 5-amino-1-(2-methylpropyl)-3-(trifluoromethyl)-1H-pyrazole-4-carboxylate

The titled compound was prepared by the reaction of Step 2 intermediate (500 mg, 1.99 mmol) with 2-bromo-1-(2,6-difluorophenyl)ethanone (561 mg, 2.38 mmol) using potassium fluoride (173 mg, 2.98 mmol) in dry DMF (5.0 mL) as per the procedure described in Step 4 of Intermediate 1 to yield 435 mg of the product as a sticky oil. ¹H NMR (300 MHz, DMSO-d₆): δ 0.84 (d, J=6.3 Hz, 6H), 2.00-2.10 (m, 1H), 3.81 (d, J=7.2 Hz, 2H), 5.25 (s, 2H), 6.76 (s, 2H), 7.26 (t, J=8.7 Hz, 2H), 7.60-7.70 (m, 1H).

### Intermediate 117

2-(4-Fluorophenyl)-2-oxoethyl 5-amino-1-ethyl-3-methyl-1H-pyrazole-4-carboxylate

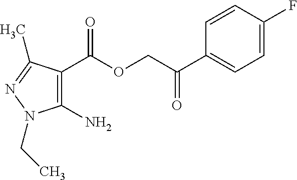 [SMILES: CCn1nc(C)c(C(=O)OCC(=O)c2ccc(F)cc2)c1N]

The titled compound was prepared by the reaction of 5-amino-1-ethyl-3-methyl-1H-pyrazole-4-carboxylic acid (500 mg, 2.95 mmol) with 2-bromo-1-[4-fluorophenyl]ethanone (770 mg, 3.54 mmol) using potassium fluoride (257 mg, 4.43 mmol) in dry DMF (5.0 mL) as per the procedure described in Step 4 of Intermediate 1 to yield 650 mg of the product as a solid. ¹H NMR (300 MHz, CDCl₃): δ 1.41 (t, J=7.2 Hz, 3H), 2.44 (s, 3H), 4.00 (q, J=7.2 Hz, 2H), 5.47 (s, 2H), 7.19 (t, J=8.7 Hz, 2H), 7.92-8.05 (m, 2H).

### Intermediate 118

2-Oxo-2-phenylethyl 5-amino-3-(4-fluorophenyl)-1-methyl-1H-pyrazole-4-carboxylate

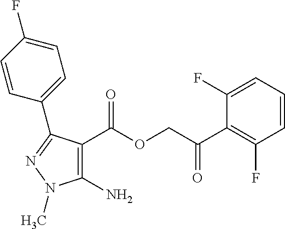 [SMILES: Cn1nc(-c2ccc(F)cc2)c(C(=O)OCC(=O)c2c(F)cccc2F)c1N]

### Step 1: 5-Amino-3-(4-fluorophenyl)-1-methyl-1H-pyrazole-4-carbonitrile

To a stirred solution of 4-fluorobenzaldehyde (2.0 g, 16.11 mmol) in methanol (80 mL) was added iodine (820 mg, 3.22 mmol) followed by malonitrile (1.1 g, 16.11 mmol) at RT and the mixture was stirred for 15 min. To this mixture were added methyl hydrazine sulfate (2.35 g, 16.11 mmol) and N,N′-diisopropylethylamine (2.75 mL, 16.11 mmol) and was stirred overnight at 60° C. The solvent was removed under vacuum and the residue was diluted with saturated sodium thiosulfate solution (30 mL). The mixture was extracted with ethyl acetate (100 mL×2). The organic layer was dried over anhydrous sodium sulfate and concentrated under reduced pressure. The residue thus obtained was purified by silica gel column chromatography to yield 470 mg of the titled product as a solid. ¹H NMR (300 MHz, DMSO-d₆): δ 3.58 (s, 3H), 6.70 (s, 2H), 7.30 (t, J=8.7 Hz, 2H), 7.79 (t, J=5.8 Hz, 2H).

### Step 2: 5-Amino-3-(4-fluorophenyl)-1-methyl-1H-pyrazole-4-carboxylic acid

The titled compound was prepared by the reaction of Step 1 intermediate (2.4 g, 11.10 mmol) with sodium hydroxide (5.0 g, 125 mmol) in water (50 mL) as per the procedure described in Step 2 of Intermediate 96 to yield 2.1 g of the desired product as a solid. ¹H NMR (300 MHz, DMSO-d₆): δ 3.57 (s, 3H), 6.32 (s, 2H), 7.16 (t, J=9.0 Hz, 2H), 7.57 (t, J=6.0 Hz, 2H), 11.85 (br s, 1H).

### Step 3: 2-Oxo-2-phenylethyl 5-amino-3-(4-fluorophenyl)-1-methyl-1H-pyrazole-4-carboxylate

The titled compound was prepared by the reaction of Step 2 intermediate (700 mg, 2.97 mmol) with 2-bromo-1-(2,6-difluorophenyl)ethanone (770 mg, 3.27 mmol) using potassium fluoride (260 mg, 4.46 mmol) in dry DMF (7.0 mL) as per the procedure described in Step 4 of Intermediate 1 to yield 490 mg of the product as a sticky oil. ¹H NMR (300 MHz, CDCl₃): δ 3.70 (s, 3H), 5.15 (s, 2H), 6.94-7.08 (m, 4H), 7.47 (t, J=8.4 Hz, 1H), 7.66 (t, J=6.0 Hz, 2H).

### Intermediate 119

2-(2-Fluoro-4-methoxyphenyl)-2-oxoethyl 5-amino-1-cyclopropyl-3-methyl-1H-pyrazole-4-carboxylate

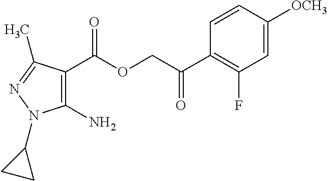 [SMILES: COc1ccc(C(=O)COC(=O)c2c(C)nn(C3CC3)c2N)c(F)c1]

The titled compound was prepared by the reaction of 5-amino-1-cyclopropyl-3-methyl-1H-pyrazole-4-carboxylic acid (200 mg, 1.10 mmol) with 2-bromo-1-(2-fluoro-4-methoxyphenyl)ethanone (327 mg, 1.32 mmol) using potassium fluoride (96 mg, 1.65 mmol) in dry DMF (2.0 mL) as per the procedure described in Step 4 of Intermediate 1 to yield 256 mg of the product as a solid. ¹H NMR (300 MHz, DMSO-d₆): δ 0.90-1.12 (m, 4H), 2.15 (s, 3H), 3.13-3.22 (m, 1H), 3.87 (s, 3H), 5.28 (s, 2H), 6.30 (s, 2H), 6.93-7.06 (m, 2H), 7.87 (t, J=8.7 Hz, 1H).

### Intermediate 120

2-(2,6-Difluorophenyl)-2-oxoethyl 5-amino-3-benzyl-1-methyl-1H-pyrazole-4-carboxylate

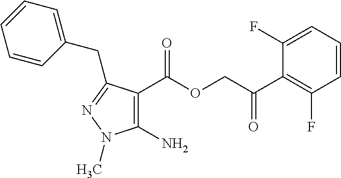 [SMILES: Cn1nc(Cc2ccccc2)c(C(=O)OCC(=O)c2c(F)cccc2F)c1N]

### Step 1: 5-Amino-3-benzyl-1-methyl-1H-pyrazole-4-carbonitrile

The titled compound was prepared by the reaction of (1-methoxy-2-phenylethylidene)propanedinitrile (6.5 g, 3.26 mmol) with methyl hydrazine sulfate (4.70 g, 3.26 mmol) using DIPEA (1.1 mL, 6.52 mmol) in dry ethanol (65 mL) as per the procedure described in Step 1 of Intermediate 75 to yield 2.45 g of the product as a solid. ¹H NMR (300 MHz, DMSO-d₆): δ 3.46 (s, 3H), 3.74 (s, 2H), 6.49 (s, 2H), 7.15-7.38 (m, 5H).

### Step 2: 5-Amino-3-benzyl-1-methyl-1H-pyrazole-4-carboxylic acid

The titled compound was prepared by the reaction of Step 1 intermediate (2.4 g, 11.30 mmol) with sodium hydroxide (5.0 g, 125 mmol) in water (50 mL) as per the procedure described in Step 2 of Intermediate 96 to yield 2.1 g of the product as a solid. ¹H NMR (300 MHz, DMSO-d6): δ 3.47 (s, 3H), 3.93 (s, 2H), 6.14 (s, 2H), 7.10-7.30 (m, 5H), 11.82 (br s, 1H).

### Step 3: 2-(2,6-Difluorophenyl)-2-oxoethyl 5-amino-3-benzyl-1-methyl-1H-pyrazole-4-carboxylate

The titled compound was prepared by the reaction of Step 2 intermediate (800 mg, 3.45 mmol) with 2-bromo-1-(2,6-difluorophenyl)ethanone (900 mg, 3.80 mmol) using potassium fluoride (300 mg, 5.17 mmol) in dry DMF (8.0 mL) as per the procedure described in Step 4 of Intermediate 1 to yield 900 mg of the product as a solid. ¹H NMR (300 MHz, DMSO-d₆): δ 3.48 (s, 3H), 3.91 (s, 2H), 5.17 (s, 2H), 6.31 (s, 2H), 7.16-7.30 (m, 6H), 7.64-7.70 (m, 2H).

### Intermediate 121

2-[2,4-Difluoro-3-(trifluoromethyl)phenyl]-2-oxoethyl 5-amino-1-ethyl-3-methyl-1H-pyrazole-4-carboxylate

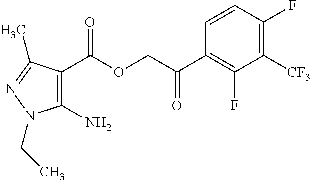 [SMILES: CCn1nc(C)c(C(=O)OCC(=O)c2ccc(F)c(C(F)(F)F)c2F)c1N]

The titled compound was prepared by the reaction of 5-amino-1-ethyl-3-methyl-1H-pyrazole-4-carboxylic acid (500 mg, 2.95 mmol) with 2-bromo-1-[2,4-difluoro-3-(trifluoromethyl)phenyl]ethanone (1.07 g, 3.54 mmol) using potassium fluoride (257 mg, 4.43 mmol) in dry DMF (5.0 mL) as per the procedure described in Step 4 of Intermediate 1 to yield 338 mg of the product as a solid. ¹H NMR (300 MHz, DMSO-d₆): δ 1.20 (t, J=7.2 Hz, 3H), 2.18 (s, 3H), 3.85 (q, J=7.2 Hz, 2H), 5.35 (s, 2H), 6.28 (s, 2H), 7.57 (t, J=10.2 Hz, 1H), 8.23-8.29 (m, 1H).

### Intermediate 122

2-(2,6-Difluorophenyl)-2-oxoethyl 5-amino-1-methyl-3-(morpholin-4-yl)-1H-pyrazole-4-carboxylate

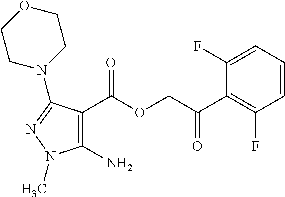 [SMILES: Cn1nc(N2CCOCC2)c(C(=O)OCC(=O)c2c(F)cccc2F)c1N]

### Step 1: [(Methylsulfanyl)(morpholin-4-yl)methylidene]propanedinitrile

To a stirred solution of 2-[bis(methylthio)methylene]malononitrile (7.3 g, 42.87 mmol) in isopropyl alcohol (50 mL), morpholine (3.8 mL, 42.87 mmol) was added and the reaction mixture was refluxed for 2 h. The solvent was evaporated under reduced pressure and the residue was stirred in diethyl ether (50 mL) for 30 min. The solid obtained was filtered and washed with diethyl ether to obtain 8.23 g of the desired product. ¹H NMR (300 MHz, CDCl₃): δ 2.62 (s, 3H), 3.75-3.95 (m, 8H).

### Step 2: 5-Amino-1-methyl-3-(morpholin-4-yl)-1H-pyrazole-4-carbonitrile

The titled compound was prepared by the reaction of Step 1 intermediate (3.8 g, 18.15 mmol) with methyl hydrazine sulfate (2.6 g, 18.15 mmol) using triethylamine (5.1 mL, 36.3 mmol) in isopropyl alcohol (40 mL) as per the procedure described in Step 1 of Intermediate 75 to yield 630 mg of the product as a solid. ¹H NMR (300 MHz, DMSO-d₆): δ 3.00-3.15 (m, 4H), 3.41 (s, 3H), 3.65-3.75 (m, 4H), 5.36 (s, 2H).

### Step 3: 5-Amino-1-methyl-3-(morpholin-4-yl)-1H-pyrazole-4-carboxylic acid

The titled compound was prepared by the reaction of Step 2 intermediate (1.4 g, 6.75 mmol) with sodium hydroxide (3.0 g, 75 mmol) in water (30 mL) as per the procedure described in Step 2 of Intermediate 96 to yield 342 mg of the product as a solid. ¹H NMR (300 MHz, DMSO-d₆): δ 3.00-3.15 (m, 4H), 3.58 (s, 3H), 3.60-3.75 (m, 4H), 5.22 (s, 2H).

### Step 4: 2-(2,6-Difluorophenyl)-2-oxoethyl 5-amino-1-methyl-3-(morpholin-4-yl)-1H-pyrazole-4-carboxylate

The titled compound was prepared by the reaction of Step 3 intermediate (340 mg, 1.50 mmol) with 2-bromo-1-(2,6-difluorophenyl)ethanone (424 mg, 1.80 mmol) using potassium fluoride (130 mg, 2.25 mmol) in dry DMF (4.0 mL) as per the procedure described in Step 4 of Intermediate 1 to yield 120 mg of the product as a solid. ¹H NMR (300 MHz, DMSO-d₆): δ 3.00-3.15 (m, 4H), 3.59 (s, 3H), 3.65-3.75 (m, 4H), 4.41 (d, J=6.0 Hz, 2H), 6.45-6.48 (m, 1H), 7.15-7.25 (m, 3H), 7.59-7.65 (m, 1H).

### Intermediate 123

2-(2,6-Difluoro-4-methoxyphenyl)-2-oxoethyl 5-amino-1-ethyl-3-methyl-1H-pyrazole-4-carboxylate

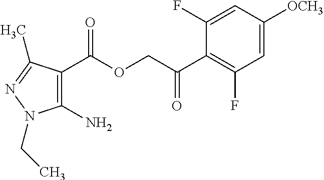 [SMILES: CCn1nc(C)c(C(=O)OCC(=O)c2c(F)cc(OC)cc2F)c1N]

The titled compound was prepared by the reaction of 5-amino-1-ethyl-3-methyl-1H-pyrazole-4-carboxylic acid (800 mg, 4.73 mmol) with 2-bromo-1-(2,6-difluoro-4-methoxyphenyl)ethanone (1.25 g, 4.73 mmol) using potassium fluoride (412 mg, 7.09 mmol) in dry DMF (6.0 mL) as per the procedure described in Step 4 of Intermediate 1 to yield 1.25 g of the product as a solid. ¹H NMR (300 MHz, DMSO-d₆): δ 1.17 (t, J=7.2 Hz, 3H), 2.12 (s, 3H), 3.79-3.95 (m, 5H), 5.11 (s, 2H), 6.24 (s, 2H), 6.87 (d, J=11.7 Hz, 2H).

### Intermediate 124

2-{2-Fluoro-4-[(methylsulfonyl)amino]phenyl}-2-oxoethyl 5-amino-1-ethyl-3-methyl-1H-pyrazole-4-carboxylate

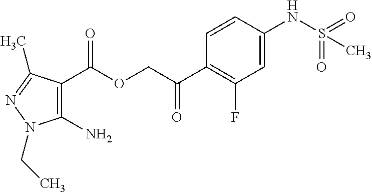 [SMILES: CCn1nc(C)c(C(=O)OCC(=O)c2ccc(NS(C)(=O)=O)cc2F)c1N]

The titled compound was prepared by the reaction of 5-amino-1-ethyl-3-methyl-1H-pyrazole-4-carboxylic acid (700 mg, 4.13 mmol) with N-[4-(bromoacetyl)-3-fluorophenyl]methanesulfonamide (1.40 g, 4.55 mmol) using potassium fluoride (360 mg, 6.20 mmol) in dry DMF (7.0 mL) as per the procedure described in Step 4 of Intermediate 1 to yield 1.10 g of the product as a solid. ¹H NMR (300 MHz, DMSO-d₆): δ 1.20 (t, J=7.2 Hz, 3H), 2.18 (s, 3H), 3.19 (s, 3H), 3.85 (q, J=7.2 Hz, 2H), 5.29 (s, 2H), 6.28 (s, 2H), 7.09-7.18 (m, 2H), 7.85-7.95 (m, 1H), 10.71 (s, 1H).

### Intermediate 125

2-{2,6-Difluoro-4-[(methylsulfonyl)amino]phenyl}-2-oxoethyl 5-amino-1-ethyl-3-methyl-1H-pyrazole-4-carboxylate

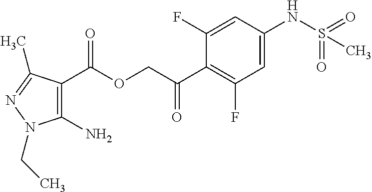 [SMILES: CCn1nc(C)c(C(=O)OCC(=O)c2c(F)cc(NS(C)(=O)=O)cc2F)c1N]

The titled compound was prepared by the reaction of 5-amino-1-ethyl-3-methyl-1H-pyrazole-4-carboxylic acid (700 mg, 4.13 mmol) with N-[4-(bromoacetyl)-3,5-difluorophenyl]methanesulfonamide (1.6 g, 4.97 mmol) using potassium fluoride (360 mg, 6.20 mmol) in dry DMF (7.0 mL) as per the procedure described in Step 4 of Intermediate 1 to yield 610 mg of the product as a solid. ¹H NMR (300 MHz, CDCl₃): δ 1.37 (t, J=6.9 Hz, 3H), 2.31 (s, 3H), 3.13 (s, 3H), 3.86 (q, J=7.8 Hz, 2H), 5.08 (br s, 2H), 5.17 (s, 2H), 6.84 (d, J=10.2 Hz, 2H).

### Intermediate 126

2-{2-Fluoro-4-[(methylsulfonyl)amino]phenyl}-2-oxoethyl 5-amino-1-methyl-3-(trifluoromethyl)-1H-pyrazole-4-carboxylate

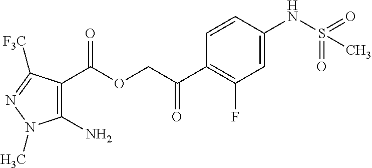 [SMILES: Cn1nc(C(F)(F)F)c(C(=O)OCC(=O)c2ccc(NS(C)(=O)=O)cc2F)c1N]

The titled compound was prepared by the reaction of 5-amino-1-methyl-3-(trifluoromethyl)-1H-pyrazole-4-carboxylic acid (650 mg, 3.11 mmol) with N-[4-(bromoacetyl)-3-fluorophenyl]methanesulfonamide (1.06 g, 3.42 mmol) using potassium fluoride (275 mg, 4.66 mmol) in dry DMF (7.0 mL) as per the procedure described in Step 4 of Intermediate 1 to yield 760 mg of the product as a solid. ¹H NMR (300 MHz, DMSO-d₆): δ 3.19 (s, 3H), 3.64 (s, 3H), 5.34 (s, 2H), 6.72 (s, 2H), 7.10-7.20 (m, 2H), 7.86-7.92 (m, 1H), 10.71 (s, 1H).

### Intermediate 127

2-[2-Fluoro-4-(2-methoxyethoxy)phenyl]-2-oxoethyl 5-amino-1-ethyl-3-methyl-1H-pyrazole-4-carboxylate

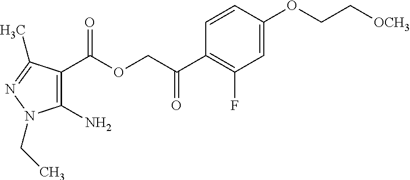 [SMILES: CCn1nc(C)c(C(=O)OCC(=O)c2ccc(OCCOC)cc2F)c1N]

The titled compound was prepared by the reaction of 5-amino-1-ethyl-3-methyl-1H-pyrazole-4-carboxylic acid (600 mg, 3.54 mmol) with 2-bromo-1-[2-fluoro-4-(2-methoxyethoxy)phenyl]ethanone (1.00 g, 3.54 mmol) using potassium fluoride (309 mg, 5.32 mmol) in dry DMF (8.0 mL) as per the procedure described in Step 4 of Intermediate 1 to yield 1.05 g of the product as a solid. ¹H NMR (300 MHz, DMSO-d₆): δ 1.20 (t, J=7.2 Hz, 3H), 2.18 (s, 3H), 3.30 (s, 3H), 3.67 (t, J=7.2 Hz, 2H), 3.85 (q, J=7.2 Hz, 2H), 4.23 (t, J=7.2 Hz, 2H), 5.29 (s, 2H), 6.29 (s, 2H), 6.95 (d, J=9.0 Hz, 1H), 7.03 (d, J=13.2 Hz, 1H), 7.85 (t, J=9.0 Hz, 1H); APCI (m/z) 380 (M+H)⁺.

### Intermediate 128

2-[4-(Cyclopropylmethoxy)-2-fluorophenyl]-2-oxoethyl 5-amino-1-ethyl-3-methyl-1H-pyrazole-4-carboxylate

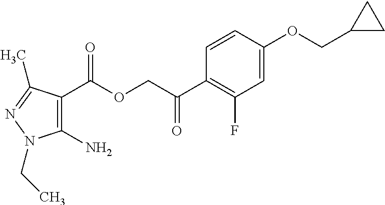 [SMILES: CCn1nc(C)c(C(=O)OCC(=O)c2ccc(OCC3CC3)cc2F)c1N]

The titled compound was prepared by the reaction of 5-amino-1-ethyl-3-methyl-1H-pyrazole-4-carboxylic acid (600 mg, 3.54 mmol) with 2-bromo-1-[4-(cyclopropylmethoxy)-2-fluorophenyl]ethanone (1.00 g, 3.54 mmol) using potassium fluoride (308 mg, 5.31 mmol) in dry DMF (6.0 mL) as per the procedure described in Step 4 of Intermediate 1 to yield 1.02 g of the product as a solid. ¹H NMR (300 MHz, DMSO-d₆): δ 0.30-0.35 (m, 2H), 0.54-0.59 (m, 2H), 1.18 (t, J=7.2 Hz, 3H), 1.21-1.25 (m, 1H), 2.15 (s, 3H), 3.83 (q, J=7.2 Hz, 2H), 3.92 (d, J=7.2 Hz, 2H), 5.26 (s, 2H), 6.25 (s, 2H), 6.85-7.00 (m, 2H), 7.82 (t, J=8.7 Hz, 1H); APCI (m/z) 375 (M+H)⁺.

### Intermediate 129

2-(2,6-Difluorophenyl)-2-oxoethyl 5-amino-1-ethyl-3-(2-methylpropyl)-1H-pyrazole-4-carboxylate

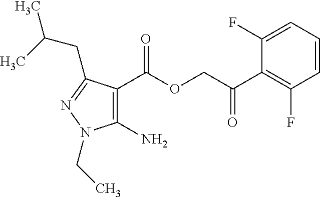 [SMILES: CCn1nc(CC(C)C)c(C(=O)OCC(=O)c2c(F)cccc2F)c1N]

### Step 1: 5-Amino-1-ethyl-3-(2-methylpropyl)-1H-pyrazole-4-carbonitrile

The titled compound was prepared by the reaction of 2-(1-methoxy-3-methylbutylidene)malononitrile (6.7 g, 40.85 mmol) with ethylhydrazine oxalate (6.1 g, 40.85 mmol) using triethylamine (11.5 mL, 81.7 mmol) in ethanol (70 mL) as per the procedure described in Step 1 of Intermediate 75 to yield 5.67 g of the product as a solid. ¹H NMR (300 MHz, DMSO-d₆): δ 0.87 (d, J=6.3 Hz, 6H), 1.16 (t, J=7.2 Hz, 3H), 1.85-1.93 (m, 1H), 2.28 (d, J=7.5 Hz, 2H), 3.82 (q, J=6.9 Hz, 2H), 6.44 (br s, 2H).

### Step 2: 5-Amino-1-ethyl-3-(2-methylpropyl)-1H-pyrazole-4-carboxamide

A solution of Step 1 intermediate (5.6 g, 29.13 mmol) in aqueous sodium hydroxide (60 mL, 11.6 g, 291.3 mmol) was stirred at 100° C. for 3 days. The mixture was cooled to RT and washed with ethyl acetate (200 mL×2). The organic layer was washed with water (200 mL and concentrated under reduced pressure to yield 4.3 g of the titled product as a solid. ¹H NMR (300 MHz, DMSO-d₆): δ 0.84 (d, J=6.9 Hz, 6H), 1.16 (t, J=6.9 Hz, 3H), 1.84-2.00 (m, 1H), 2.48 (d, J=7.2 Hz, 2H), 3.82 (q, J=6.9 Hz, 2H), 6.11 (s, 2H), 6.45 (br s, 2H).

### Step 3: 5-Amino-1-ethyl-3-(2-methylpropyl)-1H-pyrazole-4-carboxylic acid

A solution of Step 2 intermediate (4.3 g, 20.50 mmol) in aqueous sodium hydroxide (60 mL, 8.0 g, 205.0 mmol) was stirred at 100° C. for 2 days. The mixture was cooled to RT and washed with ethyl acetate (100 mL×3). The aqueous layer was acidified with 1N citric acid till pH 3-4. The aqueous mixture was extracted with ethyl acetate (100 mL×3) and the organic layer was dried over anhydrous sodium sulfate. The solution was filtered and concentrated under reduced pressure to yield 3.2 g of the titled product as oil. ¹H NMR (300 MHz, DMSO-d₆): δ 0.83 (d, J=6.9 Hz, 6H), 1.16 (t, J=6.9 Hz, 3H), 1.74-1.80 (m, 1H), 2.43 (d, J=6.9 Hz, 2H), 3.82 (q, J=6.9 Hz, 2H), 4.97 (s, 2H), 11.90 (br s, 1H).

### Step 4: 2-(2,6-Difluorophenyl)-2-oxoethyl 5-amino-1-ethyl-3-(2-methylpropyl)-1H-pyrazole-4-carboxylate

The titled compound was prepared by the reaction of Step 3 intermediate (600 mg, 2.84 mmol) with 2-bromo-1-(2,6-difluorophenyl)ethanone (801 mg, 3.41 mmol) using potassium fluoride (248 mg, 4.26 mmol) in dry DMF (6.0 mL) as per the procedure described in Step 4 of Intermediate 1 to yield 251 mg of the product as sticky oil. ¹H NMR (300 MHz, DMSO-d₆): δ 0.82 (d, J=6.9 Hz, 6H), 1.17 (t, J=6.9 Hz, 3H), 1.86-1.92 (m, 1H), 2.40 (d, J=7.2 Hz, 2H), 3.85 (q, J=6.9 Hz, 2H), 5.17 (s, 2H), 6.28 (s, 2H), 7.25 (t, J=9.0 Hz, 2H), 7.65-7.70 (m, 1H); APCI (m/z) 366 (M+H)⁺.

### Intermediate 130

2-(2-Chloro-6-fluorophenyl)-2-oxoethyl 5-amino-1-ethyl-3-methyl-1H-pyrazole-4-carboxylate

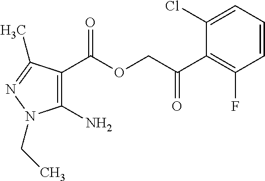 [SMILES: CCn1nc(C)c(C(=O)OCC(=O)c2c(F)cccc2Cl)c1N]

The titled compound was prepared by the reaction of 5-amino-1-ethyl-3-methyl-1H-pyrazole-4-carboxylic acid (500 mg, 2.95 mmol) with 2-bromo-1-(2-chloro-6-fluorophenyl)ethanone (820 mg, 3.25 mmol) using potassium fluoride (260 mg, 4.42 mmol) in dry DMF (5.0 mL) as per the procedure described in Step 4 of Intermediate 1 to yield 560 mg of the product as a solid. ¹H NMR (300 MHz, DMSO-d₆): δ 1.18 (t, J=7.2 Hz, 3H), 2.07 (s, 3H), 3.83 (q, J=7.2 Hz, 2H), 5.20 (s, 2H), 6.29 (s, 2H), 7.37 (d, J=8.7 Hz, 1H), 7.45 (d, J=8.4 Hz, 1H), 7.59 (t, J=8.4 Hz, 1H); APCI (m/z) 340 (M+H)⁺.

### Intermediate 131

2-(2-Chloro-6-fluorophenyl)-2-oxoethyl 5-amino-1-cyclopropyl-3-methyl-1H-pyrazole-4-carboxylate

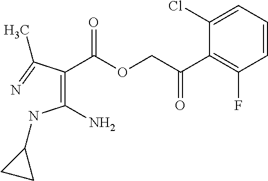 [SMILES: CC(=N)/C(C(=O)OCC(=O)c1c(F)cccc1Cl)=C(/N)NC1CC1]

The titled compound was prepared by the reaction of 5-amino-1-cyclopropyl-3-methyl-1H-pyrazole-4-carboxylic acid (300 mg, 1.65 mmol) with 2-bromo-1-(2-chloro-6-fluorophenyl)ethanone (416 mg, 1.65 mmol) using potassium fluoride (145 mg, 2.47 mmol) in dry DMF (3.0 mL) as per the procedure described in Step 4 of Intermediate 1 to yield 240 mg of the product as sticky solid. ¹H NMR (300 MHz, DMSO-d₆): δ 0.85-0.98 (m, 4H), 2.05 (s, 3H), 3.13-3.21 (m, 1H), 5.20 (s, 2H), 6.29 (br s, 2H), 7.42-7.50 (m, 2H), 7.53-7.60 (m, 1H).

### Intermediate 132

2-(2-Chlorophenyl)-2-oxoethyl 5-amino-1-cyclopropyl-3-methyl-1H-pyrazole-4-carboxylate

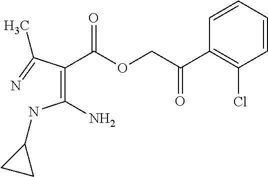 [SMILES: CC(=N)/C(C(=O)OCC(=O)c1ccccc1Cl)=C(/N)NC1CC1]

The titled compound was prepared by the reaction of 5-amino-1-cyclopropyl-3-methyl-1H-pyrazole-4-carboxylic acid (500 mg, 2.78 mmol) with 2-bromo-1-(2-chlorophenyl)ethanone (645 mg, 2.78 mmol) using potassium fluoride (245 mg, 4.17 mmol) in dry DMF (5.0 mL) as per the procedure described in Step 4 of Intermediate 1 to yield 880 mg of the product as a solid. ¹H NMR (300 MHz, DMSO-d₆): δ 0.86-0.97 (m, 4H), 2.11 (s, 3H), 3.15-3.25 (m, 1H), 5.31 (s, 2H), 6.30 (br s, 2H), 7.49-7.55 (m, 1H), 7.59 (d, J=3.3 Hz, 2H), 7.78 (d, J=7.5 Hz, 1H).

### Intermediate 133

2-(2-Chlorophenyl)-2-oxoethyl 5-amino-1-(4-fluoro-2-methylphenyl)-3-methyl-1H-pyrazole-4-carboxylate

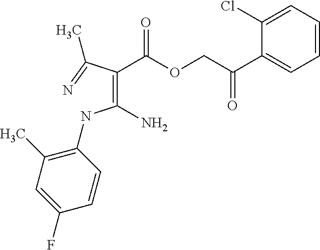 [SMILES: CC(=N)/C(C(=O)OCC(=O)c1ccccc1Cl)=C(/N)Nc1ccc(F)cc1C]

### Step 1: Ethyl 5-amino-1-(4-fluoro-2-methylphenyl)-3-methyl-1H-pyrazole-4-carboxylate

The titled compound was prepared by the reaction of ethyl (2E)-2-cyano-3-ethoxybut-2-enoate (2.0 g, 10.92 mmol) with (4-fluoro-2-methylphenyl)hydrazine hydrochloride (2.2 g, 12.01 mmol) using triethylamine (3.4 mL, 24.04 mmol) in dry ethanol (20 mL) as per the procedure described in Step 1 of Intermediate 75 to yield 2.93 g of the product as a solid. ¹H NMR (300 MHz, DMSO-d₆): δ 1.27 (t, J=7.2 Hz, 3H), 2.04 (s, 3H), 2.23 (s, 3H), 4.19 (q, J=7.2 Hz, 2H), 6.05 (br s, 2H), 7.16 (t, J=8.4 Hz, 1H), 7.24-7.35 (m, 2H).

### Step 2: 5-Amino-1-(4-fluoro-2-methylphenyl)-3-methyl-1H-pyrazole-4-carboxylic acid

The titled intermediate was prepared by the ester hydrolysis of Step 1 intermediate (1.4 g, 5.01 mmol) using aqueous solution of potassium hydroxide (2 M, 15 mL, 20.9 mmol) in ethanol (30 mL) as per the procedure described in Step 3 of Intermediate 1 to yield 1.7 g of the product as a solid. ¹H NMR (300 MHz, DMSO-d₆): δ 2.04 (s, 3H), 2.22 (s, 3H), 6.02 (s, 2H), 7.16 (t, J=8.4 Hz, 1H), 7.20-7.40 (m, 2H), 11.85 (br s, 1H).

### Step 3: 2-(2-Chlorophenyl)-2-oxoethyl 5-amino-1-(4-fluoro-2-methylphenyl)-3-methyl-1H-pyrazole-4-carboxylate

The titled compound was prepared by the reaction of Step 2 intermediate (600 mg, 2.40 mmol) with 2-bromo-1-(2-chlorophenyl)ethanone (620 mg, 2.64 mmol) using potassium fluoride (210 mg, 3.60 mmol) in dry DMF (6.0 mL) as per the procedure described in Step 4 of Intermediate 1 to yield 800 mg of the product as oil. ¹H NMR (300 MHz, DMSO-d₆): δ 2.05 (s, 3H), 2.22 (s, 3H), 5.36 (s, 2H), 6.18 (s, 2H), 7.16 (t, J=8.4 Hz, 1H), 7.30-7.40 (m, 2H), 7.45-7.57 (m, 1H), 7.58-7.62 (m, 2H), 7.80 (d, J=7.2 Hz, 1H).

### Intermediate 134

2-(2,6-Difluorophenyl)-2-oxoethyl 5-amino-1-cyclopentyl-3-methyl-1H-pyrazole-4-carboxylate

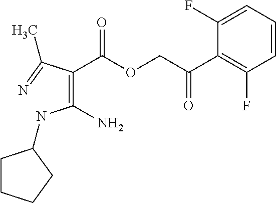 [SMILES: CC(=N)/C(C(=O)OCC(=O)c1c(F)cccc1F)=C(/N)NC1CCCC1]

### Step 1: Ethyl 5-amino-1-cyclopentyl-3-methyl-1H-pyrazole-4-carboxylate

The titled compound was prepared by the reaction of ethyl-2-cyano-3-ethoxybut-2-enoate (4.20 g, 22.87 mmol) with cyclopentylhydrazine hydrochloride (3.1 g, 22.87 mmol) using N,N-diisopropylethylamine (7.9 mL, 45.76 mmol) in dry ethanol (42 mL) as per the procedure described in Step 1 of Intermediate 75 to yield 4.08 g of the product as oil. ¹H NMR (300 MHz, DMSO-d₆): δ 1.24 (t, J=7.5 Hz, 3H), 1.46-1.62 (m, 2H), 1.67-1.98 (m, 6H), 2.16 (s, 3H), 4.14 (q, J=7.5 Hz, 2H), 4.42-4.58 (m, 1H), 6.15 (s, 2H).

### Step 2: 5-Amino-1-cyclopentyl-3-methyl-1H-pyrazole-4-carboxylic acid

The titled intermediate was prepared by the ester hydrolysis of Step 1 intermediate (4.0 g, 16.87 mmol) using potassium hydroxide (1.4 g, 25.32 mmol) in water (20 mL) and ethanol (55 mL) as per the procedure described in Step 3 of Intermediate 1 to yield 1.72 g of the product as a solid. ¹H NMR (300 MHz, DMSO-d₆): δ 1.48-1.62 (m, 2H), 1.66-1.95 (m, 6H), 2.14 (s, 3H), 4.45-4.52 (m, 1H), 6.12 (s, 2H), 11.67 (s, 1H).

### Step 3: 2-(2,6-Difluorophenyl)-2-oxoethyl 5-amino-1-cyclopentyl-3-methyl-1H-pyrazole-4-carboxylate

The titled compound was prepared by the reaction of Step 2 intermediate (1.0 g, 4.78 mmol) with 2-bromo-1-(2,6-difluorophenyl)ethanone (1.1 g, 4.78 mmol) using potassium fluoride (417 mg, 7.18 mmol) in anhydrous DMF (10 mL) as per the procedure described in Step 4 of Intermediate 1 to afford 1.25 g of the product as sticky solid. ¹H NMR (300 MHz, DMSO-d₆): δ 1.47-1.64 (m, 2H), 1.67-2.00 (m, 6H), 2.12 (s, 3H), 4.48-4.58 (m, 1H), 5.19 (s, 2H), 6.28 (s, 2H), 7.26 (t, J=8.4 Hz, 2H), 7.60-7.70 (m, 1H).

### Intermediate 135

2-(2,6-Difluorophenyl)-2-oxoethyl 5-amino-3-methyl-1-(tetrahydro-2H-pyran-4-yl)-1H-pyrazole-4-carboxylate

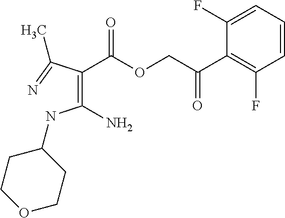 [SMILES: CC(=N)/C(C(=O)OCC(=O)c1c(F)cccc1F)=C(/N)NC1CCOCC1]

### Step 1: Ethyl 5-amino-3-methyl-1-(tetrahydro-2H-pyran-4-yl)-1H-pyrazole-4-carboxylate

The titled compound was prepared by the reaction of ethyl (2E)-2-cyano-3-ethoxybut-2-enoate (2.5 g, 13.66 mmol) with tetrahydro-2H-pyran-4-ylhydrazine hydrochloride (2.5 g, 16.39 mmol) using N,N-diisopropylethylamine (5.8 mL, 34.15 mmol) in dry ethanol (25 mL) as per the procedure described in Step 1 of Intermediate 75 to yield 2.7 g of the product as solid. ¹H NMR (300 MHz, DMSO-d₆): δ 1.24 (t, J=6.9 Hz, 3H), 1.64-1.72 (m, 2H), 1.80-2.00 (m, 2H), 2.16 (s, 3H), 3.35-3.45 (m, 2H), 3.90-3.98 (m, 2H), 4.15 (q, J=6.9 Hz, 2H), 4.20-4.34 (m, 1H), 6.23 (s, 2H).

### Step 2: 5-Amino-3-methyl-1-(tetrahydro-2H-pyran-4-yl)-1H-pyrazole-4-carboxylic acid

The titled intermediate was prepared by the ester hydrolysis of Step 1 intermediate (2.6 g, 10.26 mmol) using potassium hydroxide (1.20 g, 20.52 mmol) in water (13 mL) and ethanol (25 mL) as per the procedure described in Step 3 of Intermediate 1 to yield 1.2 g of the product as a solid. ¹H NMR (300 MHz, DMSO-d₆): δ 1.63-1.74 (m, 2H), 1.80-2.00 (m, 2H), 2.15 (s, 3H), 3.30-3.44 (m, 2H), 3.90-3.98 (m, 2H), 4.18-4.30 (m, 1H), 6.20 (s, 2H), 11.72 (br s, 1H).

### Step 3: 2-(2,6-Difluorophenyl)-2-oxoethyl 5-amino-3-methyl-1-(tetrahydro-2H-pyran-4-yl)-1H-pyrazole-4-carboxylate

The titled compound was prepared by the reaction of Step 2 intermediate (600 mg, 2.66 mmol) with 2-bromo-1-(2,6-difluorophenyl)ethanone (630 mg, 2.66 mmol) using potassium fluoride (235 mg, 3.99 mmol) in anhydrous DMF (6.0 mL) as per the procedure described in Step 4 of Intermediate 1 to afford 730 mg of the product as solid. ¹H NMR (300 MHz, DMSO-d₆): δ 1.62-1.74 (m, 2H), 1.82-2.00 (m, 2H), 2.13 (s, 3H), 3.35-3.48 (m, 2H), 3.90-4.00 (m, 2H), 4.20-4.36 (m, 1H), 5.19 (s, 2H), 6.35 (s, 2H), 7.26 (t, J=8.4 Hz, 2H), 7.65-7.68 (m, 1H).

### Intermediate 136

2-(2-Chloro-5-(pivalamidomethyl)phenyl)-2-oxoethyl 5-amino-1-ethyl-3-methyl-1H-pyrazole-4-carboxylate

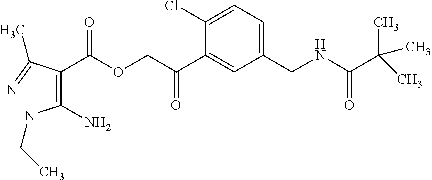 [SMILES: CCN/C(N)=C(\C(C)=N)C(=O)OCC(=O)c1cc(CNC(=O)C(C)(C)C)ccc1Cl]

The titled compound was prepared by the reaction of 5-amino-1-ethyl-3-methyl-1H-pyrazole-4-carboxylic acid (268 mg, 1.58 mmol) with N-[3-(bromoacetyl)-4-chlorobenzyl]-2,2-dimethylpropanamide (550 mg, 1.58 mmol) using potassium fluoride (138 mg, 2.38 mmol) in anhydrous DMF (3.0 mL) as per the procedure described in Step 4 of Intermediate 1 to afford 520 mg of the product as solid. ¹H NMR (300 MHz, DMSO-d₆): δ 1.12 (s, 9H), 1.19 (t, J=6.9 Hz, 3H), 2.14 (s, 3H), 3.84 (q, J=6.9 Hz, 2H), 4.27 (d, J=5.7 Hz, 2H), 5.28 (s, 2H), 6.28 (br s, 2H), 7.40 (d, J=8.4 Hz, 1H), 7.52 (d, J=8.4 Hz, 1H), 7.61 (s, 1H), 8.17 (br s, 1H).

### Intermediate 137

2-(2-Chlorophenyl)-2-oxoethyl 5-amino-1-(tetrahydro-2H-pyran-4-yl)-3-(trifluoromethyl)-1H-pyrazole-4-carboxylate

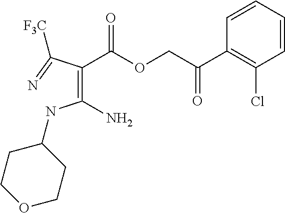 [SMILES: N=C(/C(C(=O)OCC(=O)c1ccccc1Cl)=C(/N)NC1CCOCC1)C(F)(F)F]

### Step 1: Ethyl 5-amino-1-(tetrahydro-2H-pyran-4-yl)-3-(trifluoromethyl)-1H-pyrazole-4-carboxylate

The titled compound was prepared by the reaction of ethyl-3-chloro-2-cyano-4,4,4-trifluorobut-2-enoate (8.0 g, 35.2 mmol) with tetrahydro-2H-pyran-4-ylhydrazine hydrochloride (5.3 g, 35.2 mmol) using triethylamine (12.5 mL, 87.0 mmol) in dry ethanol (80 mL) as per the procedure described in Step 1 of Intermediate 75 to yield 3.3 g of the product as solid. ¹H NMR (300 MHz, DMSO-d₆): δ 1.23 (t, J=6.9 Hz, 3H), 1.70-1.94 (m, 4H), 3.35-3.46 (m, 2H), 3.90-4.04 (m, 2H), 4.17 (q, J=6.9 Hz, 2H), 4.38-4.50 (m, 1H), 6.70 (s, 2H); APCI (m/z) 308 (M+H)⁺.

### Step 2: 5-Amino-1-(tetrahydro-2H-pyran-4-yl)-3-(trifluoromethyl)-1H-pyrazole-4-carboxylic acid

The titled intermediate was prepared by the ester hydrolysis of Step 1 intermediate (3.2 g, 10.41 mmol) using potassium hydroxide (1.15 g, 20.82 mmol) in water (10 mL) and ethanol (20 mL) as per the procedure described in Step 3 of Intermediate 1 to yield 1.5 g of the product as a solid. ¹H NMR (300 MHz, DMSO-d₆): δ 1.70-1.95 (m, 4H), 3.30-3.45 (m, 2H), 3.90-4.00 (m, 2H), 4.38-4.50 (m, 1H), 6.66 (s, 2H), 12.42 (br s, 1H); APCI (m/z) 278 (M−H)⁻.

### Step 3: 2-(2-Chlorophenyl)-2-oxoethyl 5-amino-1-(tetrahydro-2H-pyran-4-yl)-3-(trifluoromethyl)-1H-pyrazole-4-carboxylate

The titled compound was prepared by the reaction of Step 2 intermediate (600 mg, 2.14 mmol) with 2-bromo-1-(2-chlorophenyl)ethanone (500 mg, 2.14 mmol) using potassium fluoride (190 mg, 3.21 mmol) in anhydrous DMF (6.0 mL) as per the procedure described in Step 4 of Intermediate 1 to afford 1.1g of the product as solid. ¹H NMR (300 MHz, DMSO-d₆): δ 1.74-2.00 (m, 4H), 3.41 (t, J=9.6 Hz, 2H), 3.90-4.01 (m, 2H), 4.38-4.50 (m, 1H), 5.39 (s, 2H), 6.83 (s, 2H), 7.48-7.55 (m, 1H), 7.60 (d, J=3.9 Hz, 2H), 7.81 (d, J=7.8 Hz, 1H); APCI (m/z) 432 (M+H)⁺.

### Intermediate 138

2-(2,6-Difluorophenyl)-2-oxoethyl 5-amino-1-cyclobutyl-3-methyl-1H-pyrazole-4-carboxylate

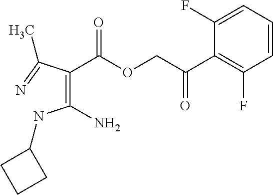 [SMILES: CC(=N)/C(C(=O)OCC(=O)c1c(F)cccc1F)=C(/N)NC1CCC1]

### Step 1: Ethyl 5-amino-1-cyclobutyl-3-methyl-1H-pyrazole-4-carboxylate

The titled compound was prepared by the reaction of ethyl ethyl-2-cyano-3-ethoxybut-2-enoate (10.0 g, 54.58 mmol) with cyclobutylhydrazine hydrochloride (6.55 g, 54.58 mmol) using N,N-diisopropylethylamine (19 mL, 109.17 mmol) in dry ethanol (65 mL) as per the procedure described in Step 1 of Intermediate 75 to yield 2.79 g of the product as solid. ¹H NMR (300 MHz, DMSO-d₆): δ 1.23 (t, J=7.2 Hz, 3H), 1.60-1.80 (m, 2H), 2.19 (s, 3H), 2.18-2.30 (m, 2H), 2.35-2.56 (m, 2H), 4.14 (q, J=7.2 Hz, 2H), 4.65-4.73 (m, 1H), 6.16 (s, 2H).

### Step 2: 5-Amino-1-cyclobutyl-3-methyl-1H-pyrazole-4-carboxylic acid

The titled intermediate was prepared by the ester hydrolysis of Step 1 intermediate (2.75 g, 12.33 mmol) using potassium hydroxide (1.40 g, 24.66 mmol) in water (20 mL) and ethanol (40 mL) as per the procedure described in Step 3 of Intermediate 1 to yield 1.72 g of the product as a solid. ¹H NMR (300 MHz, DMSO-d₆): δ 1.60-1.80 (m, 2H), 2.17 (s, 3H), 2.15-2.30 (m, 2H), 2.38-2.54 (m, 2H), 4.60-4.74 (m, 1H), 6.13 (s, 2H), 11.72 (s, 1H).

### Step 3: 2-(2,6-Difluorophenyl)-2-oxoethyl 5-amino-1-cyclobutyl-3-methyl-1H-pyrazole-4-carboxylate

The titled compound was prepared by the reaction of Step 2 intermediate (1.7 g, 8.71mmol) with 2-bromo-1-(2,6-difluorophenyl)ethanone (2.05 g, 8.71 mmol) using potassium fluoride (760 mg, 13.06 mmol) in anhydrous DMF (17 mL) as per the procedure described in Step 4 of Intermediate 1 to afford 1.97 g of the product as solid. ¹H NMR (300 MHz, DMSO-d₆): δ 1.62-1.80 (m, 2H), 2.15 (s, 3H), 2.16-2.35 (m, 2H), 2.38-2.57 (m, 2H), 4.65-4.76 (m, 1H), 5.18 (s, 2H), 6.27 (s, 2H), 7.26 (t, J=8.7 Hz, 2H), 7.62-7.73 (m, 1H).

### Intermediate 139

2-(2-Chloro-5-(pivalamidomethyl)phenyl)-2-oxoethyl 5-amino-1-methyl-3-(trifluoromethyl)-1H-pyrazole-4-carboxylate

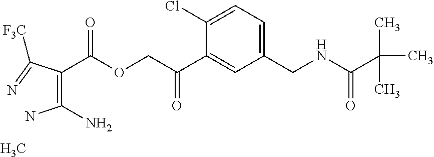

The titled compound was prepared by the reaction of 5-amino-1-methyl-3-(trifluoromethyl)-1H-pyrazole-4-carboxylic acid (482 mg, 2.30 mmol) with N-[3-(bromoacetyl)-4-chlorobenzyl]-2,2-dimethylpropanamide (800 mg, 2.30 mmol) using potassium fluoride (201 mg, 3.46 mmol) in anhydrous DMF (5.0 mL) as per the procedure described in Step 4 of Intermediate 1 to afford 780 mg of the product as solid. ¹H NMR (300 MHz, DMSO-d₆): δ 1.12 (s, 9H), 3.63 (s, 3H), 4.28 (d, J=5.7 Hz, 2H), 5.36 (s, 2H), 6.73 (br s, 2H), 7.40 (d, J=7.8 Hz, 1H), 7.54 (d, J=7.8 Hz, 1H), 7.63 (s, 1H), 8.17 (br s, 1H); APCI (m/z) 474 (M+H)⁺.

### Intermediate 140

2-(2-Chloro-5-(pivalamidomethyl)phenyl)-2-oxoethyl 5-amino-1-cyclopropyl-3-methyl-1H-pyrazole-4-carboxylate

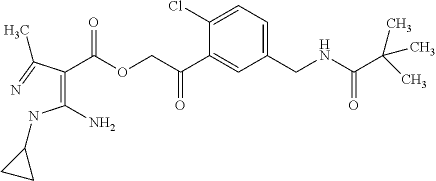 [SMILES: CC(=N)/C(C(=O)OCC(=O)c1cc(CNC(=O)C(C)(C)C)ccc1Cl)=C(/N)NC1CC1]

The titled compound was prepared by the reaction of 5-amino-1-cyclopropyl-3-methyl-1H-pyrazole-4-carboxylic acid (423 mg, 2.33 mmol) with N-[3-(bromoacetyl)-4-chlorobenzyl]-2,2-dimethylpropanamide (810 mg, 2.33 mmol) using potassium fluoride (203 mg, 3.50 mmol) in anhydrous DMF (5.0 mL) as per the procedure described in Step 4 of Intermediate 1 to afford 730 mg of the product as solid. ¹H NMR (300 MHz, DMSO-d₆): δ 0.87-0.96 (m, 4H), 1.12 (s, 9H), 2.11 (s, 3H), 3.16-3.20 (m, 1H), 4.28 (d, J=5.7 Hz, 2H), 5.28 (s, 2H), 6.30 (br s, 2H), 7.39 (d, J=8.7 Hz, 1H), 7.53 (d, J=8.1 Hz, 1H), 7.61 (s, 1H), 8.17 (br s, 1H).

### Intermediate 141

2-(2,6-Difluorophenyl)-2-oxoethyl 5-amino-1-(tetrahydro-2H-pyran-4-yl)-3-(trifluoromethyl)-1H-pyrazole-4-carboxylate

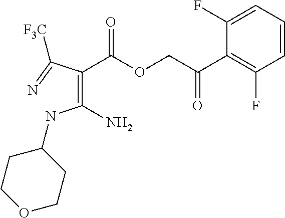 [SMILES: N=C(/C(C(=O)OCC(=O)c1c(F)cccc1F)=C(/N)NC1CCOCC1)C(F)(F)F]

The titled compound was prepared by the reaction of 5-amino-1-(tetrahydro-2H-pyran-4-yl)-3-(trifluoromethyl)-1H-pyrazole-4-carboxylic acid (750 mg, 2.68 mmol) with 2-bromo-1-(2,6-difluorophenyl)ethanone (635 mg, 2.68 mmol) using potassium fluoride (235 mg, 4.02 mmol) in anhydrous DMF (7.5 mL) as per the procedure described in Step 4 of Intermediate 1 to afford 855 mg of the product as solid. ¹H NMR (300 MHz, DMSO-d₆): δ 1.75-1.95 (m, 4H), 3.34-3.48 (m, 2H), 3.90-4.02 (m, 2H), 4.40-4.52 (m, 1H), 5.26 (s, 2H), 6.83 (s, 2H), 7.26 (t, J=8.7 Hz, 2H), 7.62-7.78 (m, 1H); APCI (m/z) 434 (M+H)⁺.

### Intermediate 142

2-(2-Fluoro-5-(pivalamidomethyl)phenyl)-2-oxoethyl 5-amino-1-methyl-3-(trifluoromethyl)-1H-pyrazole-4-carboxylate

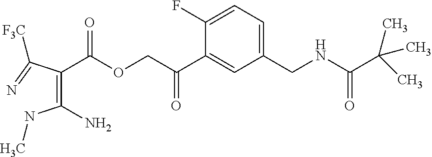 [SMILES: CN/C(N)=C(\C(=N)C(F)(F)F)C(=O)OCC(=O)c1cc(CNC(=O)C(C)(C)C)ccc1F]

The titled compound was prepared by the reaction of 5-amino-1-methyl-3-(trifluoromethyl)-1H-pyrazole-4-carboxylic acid (950 mg, 4.54 mmol) with N-[3-(bromoacetyl)-4-fluorobenzyl]-2,2-dimethylpropanamide (1.5 g, 4.54 mmol) using potassium fluoride (396 mg, 6.82 mmol) in anhydrous DMF (10 mL) as per the procedure described in Step 4 of Intermediate 1 to afford 1.57 g of the product as a solid. ¹H NMR (300 MHz, DMSO-d₆): δ 1.12 (s, 9H), 3.64 (s, 3H), 4.28 (d, J=5.7 Hz, 2H), 5.37 (s, 2H), 6.73 (br s, 2H), 7.37 (t, J=9.3 Hz, 1H), 7.50-7.60 (m, 1H), 7.73 (d, J=7.8 Hz, 1H), 8.17 (br s, 1H).

### Intermediate 143

2-(2,6-Difluorophenyl)-2-oxoethyl 5-amino-1-cyclohexyl-3-methyl-1H-pyrazole-4-carboxylate

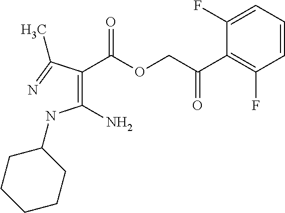 [SMILES: CC(=N)/C(C(=O)OCC(=O)c1c(F)cccc1F)=C(/N)NC1CCCCC1]

### Step 1: Ethyl 5-amino-1-cyclohexyl-3-methyl-1H-pyrazole-4-carboxylate

The titled compound was prepared by the reaction of ethyl (2E)-2-cyano-3-ethoxybut-2-enoate (5.60 g, 37.60 mmol) with cyclohexylhydrazine hydrochloride (6.9 g, 37.60 mmol) using N,N-Diisopropylethylamine (13 mL, 75.37 mmol) in ethanol (56 mL) as per the procedure described in Step 1 of Intermediate 75 to yield 7.1 g of the product as oil. ¹H NMR (300 MHz, DMSO-d₆): δ 1.10-1.40 (m, 2H), 1.23 (t, J=6.9 Hz, 3H), 1.42-1.85 (m, 8H), 2.15 (s, 3H), 3.95-4.00 (m, 1H), 4.14 (q, J=6.9 Hz, 2H), 6.15 (s, 2H); APCI (m/z) 252 (M+H)⁺.

### Step 2: 5-Amino-1-cyclohexyl-3-methyl-1H-pyrazole-4-carboxylic acid

The titled intermediate was prepared by the ester hydrolysis of Step 1 intermediate (7.0 g, 27.85 mmol) using potassium hydroxide (2.4 g, 41.78 mmol) in water (35 mL) and ethanol (90 mL) as per the procedure described in Step 3 of Intermediate 1 to yield 2.97 g of the product as a solid. ¹H NMR (300 MHz, DMSO-d₆): δ 1.11-1.42 (m, 3H), 1.58-1.82 (m, 7H), 2.13 (s, 3H), 3.90-4.07 (m, 1H), 6.13 (s, 2H), 11.67 (s, 1H).

### Step 3: 2-(2,6-Difluorophenyl)-2-oxoethyl 5-amino-1-cyclohexyl-3-methyl-1H-pyrazole-4-carboxylate

The titled compound was prepared by the reaction of Step 2 intermediate (1.0 g, 4.47 mmol) with 2-bromo-1-(2,6-difluorophenyl)ethanone (1.05 g, 4.47 mmol) using potassium fluoride (390 mg, 6.79 mmol) in anhydrous DMF (10 mL) as per the procedure described in Step 4 of Intermediate 1 to afford 1.12 g of the product as sticky oil. ¹H NMR (300 MHz, DMSO-d₆): δ 1.10-1.45 (m, 3H), 1.57-1.82 (m, 7H), 2.12 (s, 3H), 3.96-4.15 (m, 1H), 5.18 (s, 2H), 6.27 (s, 2H), 7.26 (t, J=8.4 Hz, 2H), 7.62-7.72 (m, 1H); APCI (m/z) 378 (M+H)⁺.

### Intermediate 144

2-(2,6-Difluorophenyl)-2-oxoethyl 5-amino-1-(4,4-difluorocyclohexyl)-3-methyl-1H-pyrazole-4-carboxylate

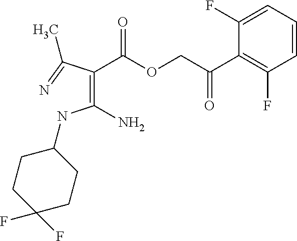 [SMILES: CC(=N)/C(C(=O)OCC(=O)c1c(F)cccc1F)=C(/N)NC1CCC(F)(F)CC1]

### Step 1: Ethyl 5-amino-1-(4,4-difluorocyclohexyl)-3-methyl-1H-pyrazole-4-carboxylate

The titled compound was prepared by the reaction of ethyl -2-cyano-3-ethoxybut-2-enoate (2.78 g, 15.21 mmol) with (4,4-difluorocyclohexyl)hydrazine hydrochloride (2.8 g, 15.21 mmol) using N,N-Diisopropylethylamine (5.2 mL, 30.42 mmol) in ethanol (28 mL) as per the procedure described in Step 1 of Intermediate 75 to yield 4.3 g of the product as oil. ¹H NMR (300 MHz, DMSO-d₆): δ 1.24 (t, J=7.2 Hz, 3H), 1.90-2.01 (m, 4H), 2.08-2.20 (m, 4H), 2.16 (s, 3H), 3.95-4.00 (m, 1H), 4.15 (q, J=6.9 Hz, 3H), 6.22 (s, 2H).

### Step 2: 5-Amino-1-(4,4-difluorocyclohexyl)-3-methyl-1H-pyrazole-4-carboxylic acid

The titled intermediate was prepared by the ester hydrolysis of Step 1 intermediate (4.3 g, 15.00 mmol) using potassium hydroxide (2.6 g, 46.42 mmol) in water (18 mL) and ethanol (50 mL) as per the procedure described in Step 3 of Intermediate 1 to yield 1.9 g of the product as a solid. ¹H NMR (300 MHz, DMSO-d₆): δ 1.75-2.02 (m, 6H), 2.10-2.20 (m, 2H), 2.12 (s, 3H), 4.14-4.17 (m, 1H), 6.16 (s, 2H), 11.71 (s, 1H).

### Step 3: 2-(2,6-Difluorophenyl)-2-oxoethyl 5-amino-1-(4,4-difluorocyclohexyl)-3-methyl-1H-pyrazole-4-carboxylate

The titled compound was prepared by the reaction of Step 2 intermediate (1.0 g, 3.86 mmol) with 2-bromo-1-(2,6-difluorophenyl)ethanone (907 mg, 3.86 mmol) using potassium fluoride (336 mg, 5.79 mmol) in anhydrous DMF (10 mL) as per the procedure described in Step 4 of Intermediate 1 to afford 1.23 g of the product as a sticky solid. ¹H NMR (300 MHz, DMSO-d₆): δ 1.78-2.05 (m, 6H), 2.09-2.12 (m, 2H), 2.11 (s, 3H), 4.17-4.21 (m, 1H), 5.17 (s, 2H), 6.32 (s, 2H), 7.24 (t, J=8.7 Hz, 2H), 7.63-7.67 (m, 1H).

### Intermediate 145

2-(2-Fluoro-5-(pivalamidomethyl)phenyl)-2-oxoethyl 5-amino-1-ethyl-3-methyl-1H-pyrazole-4-carboxylate

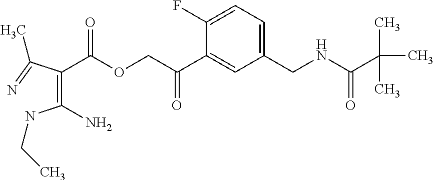 [SMILES: CCN/C(N)=C(\C(C)=N)C(=O)OCC(=O)c1cc(CNC(=O)C(C)(C)C)ccc1F]

The titled compound was prepared by the reaction of 5-amino-1-ethyl-3-methyl-1H-pyrazole-4-carboxylic acid (410 mg, 2.42 mmol) with N-[3-(bromoacetyl)-4-fluorobenzyl]-2,2-dimethylpropanamide (800 mg, 2.42 mmol) using potassium fluoride (210 mg, 3.63 mmol) in anhydrous DMF (5.0 mL) as per the procedure described in Step 4 of Intermediate 1 to afford 730 mg of the product as a solid. ¹H NMR (300 MHz, DMSO-d₆): δ 1.12 (s, 9H), 1.23 (t, J=6.9 Hz, 3H), 2.18 (s, 3H), 3.85 (q, J=6.9 Hz, 2H), 4.28 (d, J=5.4 Hz, 2H), 5.32 (s, 2H), 6.28 (s, 2H), 7.36 (t, J=9.3 Hz, 1H), 7.53-7.58 (m, 1H), 7.73-7.76 (m, 1H), 8.16 (br s, 1H).

### Intermediate 146

2-(2,6-Difluorophenyl)-2-oxoethyl 5-amino-1-(2-chloro-4-fluorophenyl)-3-methyl-1H-pyrazole-4-carboxylate

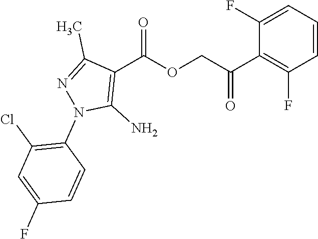 [SMILES: Cc1nn(-c2ccc(F)cc2Cl)c(N)c1C(=O)OCC(=O)c1c(F)cccc1F]

### Step 1: Ethyl 5-amino-1-(2-chloro-4-fluorophenyl)-3-methyl-1H-pyrazole-4-carboxylate

The titled compound was prepared by the reaction of ethyl (2E)-2-cyano-3-ethoxybut-2-enoate (2.32 g, 12.68 mmol) with (2-chloro-4-fluorophenyl)hydrazine hydrochloride (2.5 g, 12.68 mmol) using triethylamine (3.9 mL, 27.89 mmol) in ethanol (25 mL) as per the procedure described in Step 1 of Intermediate 75 to yield 3.57 g of the product as a solid. ¹H NMR (300 MHz, DMSO-d₆): δ 1.27 (t, J=6.9 Hz, 3H), 2.22 (s, 3H), 4.19 (q, J=6.9 Hz, 2H), 6.24 (s, 2H), 7.37 (t, J=8.4 Hz, 1H), 7.50-7.62 (m, 1H), 7.67-7.74 (m, 1H).

### Step 2: 5-Amino-1-(2-chloro-4-fluorophenyl)-3-methyl-1H-pyrazole-4-carboxylic acid

The titled intermediate was prepared by the ester hydrolysis of Step 1 intermediate (3.5 g, 11.75 mmol) using potassium hydroxide (1.31 g, 23.51 mmol) in water (17.5 mL) and ethanol (35 mL) as per the procedure described in Step 3 of Intermediate 1 to yield 2.8 g of the product as a solid. ¹H NMR (300 MHz, DMSO-d₆): δ 2.21 (s, 3H), 6.20 (s, 2H), 7.37 (t, J=8.4 Hz, 1H), 7.50-7.60 (m, 1H), 7.65-7.74 (m, 1H), 11.95 (br s, 1H).

### Step 3: 2-(2,6-Difluorophenyl)-2-oxoethyl 5-amino-1-(2-chloro-4-fluorophenyl)-3-methyl-1H-pyrazole-4-carboxylate

The titled compound was prepared by the reaction of Step 2 intermediate (1.0 g, 3.70 mmol) with 2-bromo-1-(2,6-difluorophenyl)ethanone (871mg, 3.70 mmol) using potassium fluoride (323 mg, 5.56 mmol) in anhydrous DMF (10 mL) as per the procedure described in Step 4 of Intermediate 1 to afford 980 mg of the product as a solid. ¹H NMR (300 MHz, DMSO-d₆): δ 2.20 (s, 3H), 5.25 (s, 2H), 6.38 (s, 2H), 7.28 (t, J=8.4 Hz, 2H), 7.33-7.44 (m, 1H), 7.54-7.76 (m, 3H); APCI (m/z) 424 (M+H)⁺.

### Intermediate 147

2-(2,6-Difluorophenyl)-2-oxoethyl 5-amino-1-cyclopropyl-3-ethyl-1H-pyrazole-4-carboxylate

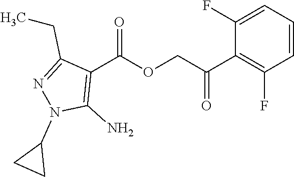 [SMILES: CCc1nn(C2CC2)c(N)c1C(=O)OCC(=O)c1c(F)cccc1F]

### Step 1: Ethyl 5-amino-1-cyclopropyl-3-ethyl-1H-pyrazole-4-carboxylate

The titled compound was prepared by the reaction of ethyl ethyl-2-cyano-3-ethoxypent-2-enoate (3.2 g, 16.22 mmol) with cyclopropylhydrazine hydrochloride (2.11 g, 19.46 mmol) using N,N-diisopropylethylamine (5.6 mL, 32.44 mmol) in dry ethanol (32 mL) as per the procedure described in Step 1 of Intermediate 75 to yield 2.35 g of the product as oil. ¹H NMR (300 MHz, DMSO-d₆): δ 0.87-0.93 (m, 2H), 1.07 (t, J=7.5 Hz, 3H), 1.23 (t, J=6.9 Hz, 3H), 1.35-1.44 (m, 2H), 2.56 (q, J=7.5 Hz, 2H), 3.13-3.17 (m, 1H), 4.15 (q, J=6.9 Hz, 2H), 6.16 (s, 2H); APCI (m/z) 224 (M+H)⁺.

### Step 2: 5-amino-1-cyclopropyl-3-ethyl-1H-pyrazole-4-carboxylic acid

The titled intermediate was prepared by the ester hydrolysis of Step 1 intermediate (2.3 g, 10.30 mmol) using potassium hydroxide (1.15 g, 20.6 mmol) in water (3.0 mL) and ethanol (23 mL) as per the procedure described in Step 3 of Intermediate 1 to yield 1.10 g of the product as a solid. ¹H NMR (300 MHz, DMSO-d₆): δ 0.87-0.94 (m, 4H), 1.07 (t, J=7.8 Hz, 3H), 2.55 (q, J=7.8 Hz, 2H), 3.13-3.17 (m, 1H), 6.12 (s, 2H), 11.80 (br s, 1H).

### Step 3: 2-(2,6-Difluorophenyl)-2-oxoethyl 5-amino-1-cyclopropyl-3-ethyl-1H-pyrazole-4-carboxylate

The titled compound was prepared by the reaction of Step 2 intermediate (950 mg, 4.36 mmol) with 2-bromo-1-(2,6-difluorophenyl)ethanone (1.15 g, 4.86 mmol) using potassium fluoride (430 mg, 7.3 mmol) in anhydrous DMF (10 mL) as per the procedure described in Step 4 of Intermediate 1 to afford 1.05 g of the product as sticky oil. ¹H NMR (300 MHz, DMSO-d₆): δ 0.88-1.09 (m, 4H), 1.05 (t, J=7.8 Hz, 3H), 2.55 (q, J=7.8 Hz, 2H), 3.15-3.19 (m, 1H), 5.19 (s, 2H), 6.30 (s, 2H), 7.26 (t, J=8.7 Hz, 2H), 7.62-7.72 (m, 1H); APCI (m/z) 350 (M+H)⁺.

### Intermediate 148

2-(2-Chlorophenyl)-2-oxoethyl 5-amino-1-(2-chloro-4-fluorophenyl)-3-methyl-1H-pyrazole-4-carboxylate

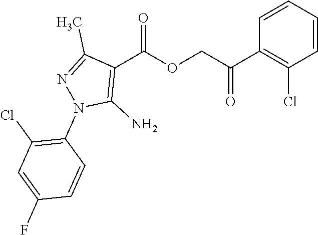 [SMILES: Cc1nn(-c2ccc(F)cc2Cl)c(N)c1C(=O)OCC(=O)c1ccccc1Cl]

The titled compound was prepared by the reaction of 5-amino-1-(2-chloro-4-fluorophenyl)-3-methyl-1H-pyrazole-4-carboxylic acid (1.0 g, 3.70 mmol) with 2-bromo-1-(2-chlorophenyl)ethanone (866 mg, 3.70 mmol) using potassium fluoride (323 mg, 5.56 mmol) in anhydrous DMF (10 mL) as per the procedure described in Step 4 of Intermediate 1 to afford 1.01 g of the product as a solid. ¹H NMR (300 MHz, DMSO-d₆): δ 2.21 (s, 3H), 5.36 (s, 2H), 6.39 (s, 2H), 7.38 (t, J=8.4 Hz, 1H), 7.52-7.61 (m, 4H), 7.69-7.74 (m, 1H), 7.80 (d, J=7.8 Hz, 1H); APCI (m/z) 424 (M+H)⁺.

### Intermediate 149

2-(2-Chloro-5-(pivalamidomethyl)phenyl)-2-oxoethyl 5-amino-3-methyl-1-(tetrahydro-2H-pyran-4-yl)-1H-pyrazole-4-carboxylate

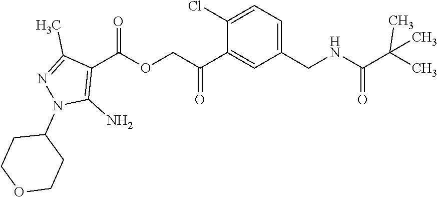 [SMILES: Cc1nn(C2CCOCC2)c(N)c1C(=O)OCC(=O)c1cc(CNC(=O)C(C)(C)C)ccc1Cl]

The titled compound was prepared by the reaction of 5-amino-3-methyl-1-(tetrahydro-2H-pyran-4-yl)-1H-pyrazole-4-carboxylic acid (470 mg, 2.08 mmol) with N-[3-(bromoacetyl)-4-chlorobenzyl]-2,2-dimethylpropanamide (723 mg, 2.08 mmol) using potassium fluoride (181 mg, 3.13 mmol) in anhydrous DMF (5.0 mL) as per the procedure described in Step 4 of Intermediate 1 to afford 705 mg of the product as a solid. ¹H NMR (300 MHz, DMSO-d₆): δ 1.12 (s, 9H), 1.67-1.73 (m, 2H), 1.80-2.00 (m, 3H), 2.16 (s, 3H), 3.35-3.48 (m, 2H), 3.91-3.98 (m, 2H), 4.28 (d, J=6.0 Hz, 2H), 5.28 (s, 2H), 6.35 (s, 2H), 7.40 (d, J=8.7 Hz, 1H), 7.53 (d, J=7.8 Hz, 1H), 7.61 (s, 1H), 8.17 (s, 1H); APCI (m/z) 491 (M+H)⁺.

### Intermediate 150

2-Oxo-2-(2-(trifluoromethyl)phenyl)ethyl 5-amino-1-ethyl-3-methyl-1H-pyrazole-4-carboxylate

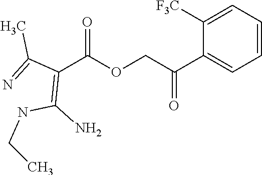 [SMILES: CCN/C(N)=C(\C(C)=N)C(=O)OCC(=O)c1ccccc1C(F)(F)F]

The titled compound was prepared by the reaction of 5-amino-1-ethyl-3-methyl-1H-pyrazole-4-carboxylic acid (800 mg, 4.72 mmol) with 2-bromo-1-[2-(trifluoromethyl)phenyl]ethanone (1.11 g, 4.72 mmol) using potassium fluoride (411 mg, 7.08 mmol) in anhydrous DMF (8.0 mL) as per the procedure described in Step 4 of Intermediate 1 to afford 1.23 g of the product as a solid. ¹H NMR (300 MHz, DMSO-d₆): δ 1.19 (t, J=6.9 Hz, 3H), 2.10 (s, 3H), 3.84 (q, J=6.9 Hz, 2H), 5.31 (s, 2H), 6.29 (s, 2H), 7.79-7.95 (m, 4H).

### Intermediate 151

2-(2-Chlorophenyl)-2-oxoethyl 5-amino-1-cyclobutyl-3-methyl-1H-pyrazole-4-carboxylate

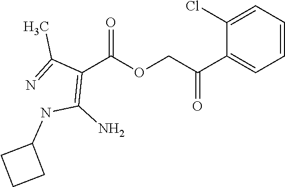 [SMILES: CC(=N)/C(C(=O)OCC(=O)c1ccccc1Cl)=C(/N)NC1CCC1]

The titled compound was prepared by the reaction of 5-amino-1-cyclobutyl-3-methyl-1H-pyrazole-4-carboxylic acid (1.85 g, 9.48 mmol) with 2-bromo-1-(2-chlorophenyl)ethanone (2.45 g, 10.43 mmol) using potassium fluoride (830 mg, 14.23 mmol) in anhydrous DMF (15 mL) as per the procedure described in Step 4 of Intermediate 1 to afford 2.06 g of the product as a solid. ¹H NMR (300 MHz, DMSO-d₆): δ 1.62-2.00 (m, 2H), 2.17 (s, 3H), 2.18-2.34 (m, 2H), 2.37-2.56 (m, 2H), 4.65-4.72 (m, 1H), 5.30 (s, 2H), 6.27 (s, 2H), 7.46-7.62 (m, 3H), 7.76 (d, J=7.2 Hz, 1H).

### Intermediate 152

2-(2-Chlorophenyl)-2-oxoethyl 5-amino-3-(2-chlorobenzyl)-1-methyl-1H-pyrazole-4-carboxylate

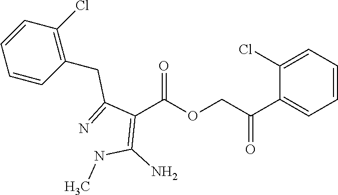 [SMILES: CN/C(N)=C(\C(=N)Cc1ccccc1Cl)C(=O)OCC(=O)c1ccccc1Cl]

### Step 1: 5-Amino-3-(2-chlorobenzyl)-1-methyl-1H-pyrazole-4-carbonitrile

The titled compound was prepared by the reaction of [2-(2-chlorophenyl)-1-methoxyethylidene]propanedinitrile (2.8 g, 12.03 mmol) with methylhydrazine sulfate (1.73 g, 12.03 mmol) using N,N-Diisopropylethylamine (4.2 mL, 24.07 mmol) in dry ethanol (30 mL) as per the procedure described in Step 1 of Intermediate 75 to yield 1.55 g of the product as a solid. ¹H NMR (300 MHz, DMSO-d₆): □ 3.44 (s, 3H), 3.88 (s, 2H), 6.53 (s, 2H), 7.27 (s, 3H), 7.39-7.42 (m, 1H); APCI (m/z) 245 (M−H)⁻.

### Step 2: 5-Amino-3-(2-chlorobenzyl)-1-methyl-1H-pyrazole-4-carboxylic acid

A mixture of Step 1 intermediate (2.35 g, 9.52 mmol) and sodium hydroxide (3.85 g, 95.2 mmol) in water (40 mL) was refluxed for 72 h. The mixture was cooled to RT and washed with ethyl acetate (2×75 mL). The aqueous layer was collected and acidified with 1N citric acid. The precipitated solid was filtered and washed with water (20 mL). The solid was dried under vacuum to afford 2.06 g of the titled product. ¹H NMR (300 MHz, DMSO-d₆): □ 3.45 (s, 3H), 4.04 (s, 2H), 6.19 (s, 2H), 7.08 (s, 1H), 7.18-7.22 (m, 2H), 7.35-7.40 (m, 1H), 11.08 (br s, 1H); APCI (m/z) 264 (M−H)⁻.

### Step 3: 2-(2-Chlorophenyl)-2-oxoethyl 5-amino-3-(2-chlorobenzyl)-1-methyl-1H-pyrazole-4-carboxylate

The titled compound was prepared by the reaction of Step 2 intermediate (1.0 g, 3.76 mmol) with 2-bromo-1-(2-chlorophenyl)ethanone (970 mg, 4.14 mmol) using potassium fluoride (330 mg, 5.04 mmol) in anhydrous DMF (10 mL) as per the procedure described in Step 4 of Intermediate 1 to afford 1.3 g of the product as a solid. ¹H NMR (300 MHz, DMSO- d6): □ 3.47 (s, 3H), 4.05 (s, 2H), 5.27 (s, 2H), 6.34 (s, 2H), 7.11-7.14 (m, 1H), 7.15-7.25 (m, 2H), 7.32-7.52 (m, 2H), 7.54-7.57 (m, 2H), 7.73 (d, J=7.8 Hz, 1H); APCI (m/z) 418 (M)⁺.

### Intermediate 153

2-(2-Chlorophenyl)-2-oxoethyl 5-amino-3-ethyl-1-(2-morpholinoethyl)-1H-pyrazole-4-carboxylate

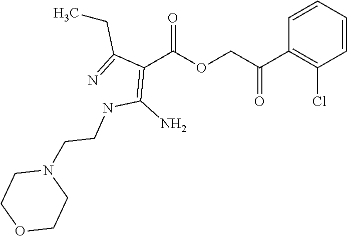 [SMILES: CCC(=N)/C(C(=O)OCC(=O)c1ccccc1Cl)=C(/N)NCCN1CCOCC1]

### Step 1: Ethyl 5-amino-3-ethyl-1-(2-morpholinoethyl)-1H-pyrazole-4-carboxylate

The titled compound was prepared by the reaction of ethyl-2-cyano-3-ethoxypent-2-enoate (4.0 g, 20.304 mmol) with 4-(2-hydrazinylethyl)morpholine (3.5 g, 24.36 mmol) using N,N-Diisopropylethylamine (6.9 mL, 40.60 mmol) in ethanol (45 mL) as per the procedure described in Step 1 of Intermediate 75 to yield 2.85 g of the product as a liquid. ¹H NMR (300 MHz, DMSO-d₆): δ 1.08 (t, J=7.2 Hz, 3H), 1.24 (t, J=6.3 Hz, 3H), 2.37-2.45 (m, 4H), 2.47-2.64 (m, 4H), 3.53-3.59 (m, 4H), 3.94 (t, J=6.3 Hz, 2H), 4.15 (q, J=7.2 Hz, 2H), 6.26 (s, 2H).

### Step 2: 5-Amino-3-ethyl-1-(2-morpholinoethyl)-1H-pyrazole-4-carboxylic acid

The titled intermediate was prepared by the ester hydrolysis of Step 1 intermediate (2.8 g, 9.45 mmol) using aqueous solution of potassium hydroxide (2.0 M, 20 mL, 37.918 mmol) and ethanol (20 mL) as per the procedure described in Step 2 of Intermediate 91 to yield 2.05 g of the product as a sticky solid. ¹H NMR (300 MHz, DMSO-d₆): δ 1.08 (t, J=7.8 Hz, 3H), 2.28-2.34 (m, 4H), 2.48-2.62 (m, 4H), 3.51-3.58 (m, 4H), 3.84-3.97 (m, 2H), 6.23 (s, 2H), 11.74 (br. S, 1H).

### Step 3: 2-(2-Chlorophenyl)-2-oxoethyl 5-amino-3-ethyl-1-(2-morpholinoethyl)-1H-pyrazole-4-carboxylate

The titled compound was prepared by the reaction of Step 2 intermediate (2.0 g, 7.46 mmol) with 2-bromo-1-(2-chlorophenyl)ethanone (1.9 g, 8.20 mmol) using potassium fluoride (650 mg, 11.19 mmol) in dry DMF (20 mL) as per the procedure described in Step 4 of Intermediate 1 to yield 2.24 g of the product as thick liquid. ¹H NMR (300 MHz, DMSO-d₆): δ 1.05 (t, J=7.2 Hz, 3H), 2.38-2.47 (m, 4H), 2.49-2.63 (m, 4H), 3.52-3.59 (m, 4H), 3.94 (t, J=7.2 Hz, 2H), 5.29 (s, 2H), 6.37 (s, 2H), 7.40-7.52 (m, 1H), 7.56 (s, 2H), 7.76 (d, J=7.2 Hz, 1H), 8.29 (s, 1H).

### Intermediate 154

2-(2-Chlorophenyl)-2-oxoethyl 5-amino-3-methyl-1-(3-morpholinopropyl)-1H-pyrazole-4-carboxylate

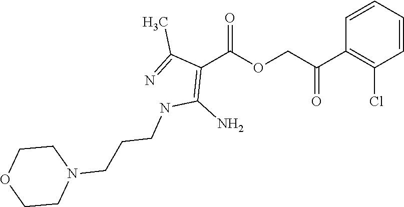 [SMILES: CC(=N)/C(C(=O)OCC(=O)c1ccccc1Cl)=C(/N)NCCCN1CCOCC1]

### Step 1: Ethyl 5-amino-3-methyl-1-(3-morpholinopropyl)-1H-pyrazole-4-carboxylate

The titled compound was prepared by the reaction of ethyl (2E)-2-cyano-3-ethoxybut-2-enoate (5.7 g, 31.10 mmol) with 4-(3-hydrazinylpropyl)morpholine (6.0 g, 37.70 mmol) using N,N-Diisopropylethylamine (21.4 mL, 12.40 mmol) in dry ethanol (57 mL) as per the procedure described in Step 1 of Intermediate 75 to yield 3.12 g of the product. ¹H NMR (300 MHz, CDCl₃): δ 1.34 (t, J=7.2 Hz, 3H), 1.98 (t, J=7.2 Hz, 2H), 2.18-2.30 (m, 2H), 2.32 (s, 3H), 2.38-2.50 (m, 4H), 3.65-3.78 (m, 4H), 3.93 (t, J=5.7 Hz, 2H), 4.27 (q, J=7.2 Hz, 2H), 6.14 (s, 2H).

### Step 2: 5-Amino-3-methyl-1-(3-morpholinopropyl)-1H-pyrazole-4-carboxylic acid

The titled intermediate was prepared by the ester hydrolysis of Step 1 intermediate (3.10 g, 10.46 mmol) using aqueous solution of potassium hydroxide (2 M, 2.34 g, 41.70 mmol in 20 mL water) in ethanol (20 mL) as per the procedure described in Step 3 of Intermediate 1 to yield 740 mg of the product as a white solid. ¹H NMR (300 MHz, DMSO-d₆): δ 1.68-1.86 (m, 2H), 2.14 (s, 3H), 2.16-2.27 (m, 2H), 2.28-2.40 (m, 4H), 3.50-3.63 (m, 4H), 3.80 (t, J=6.3 Hz, 2H), 6.15 (br s, 2H).

### Step 3: 2-(2-Chlorophenyl)-2-oxoethyl 5-amino-3-methyl-1-(3-morpholinopropyl)-1H-pyrazole-4-carboxylate

The titled compound was prepared by the reaction of Step 2 intermediate (720 mg, 2.68 mmol) with 2-bromo-1-(2-chlorophenyl)ethanone (690 mg, 2.95 mmol) using potassium fluoride (234 mg, 4.02 mmol) in dry DMF (9.5 mL) as per the procedure described in Step 4 of Intermediate 1 to yield 537 mg of the product. ¹H NMR (300 MHz, DMSO-d₆): δ 1.75-1.87 (m, 2H), 2.13(s, 3H), 2.20-2.38 (m, 6H), 3.54-3.60 (m, 4H), 3.82 (t, J=6.5 Hz, 2H), 5.30 (s, 2H), 6.31 (s, 2H), 7.43-7.63 (m, 3H), 7.77 (d, J=6.6 Hz, 1H).

### Intermediate 155

2-(2-Chlorophenyl)-2-oxoethyl 5-amino-1-(2-((2R,6S)-2,6-dimethylmorpholino)ethyl)-3-methyl-1H-pyrazole-4-carboxylate

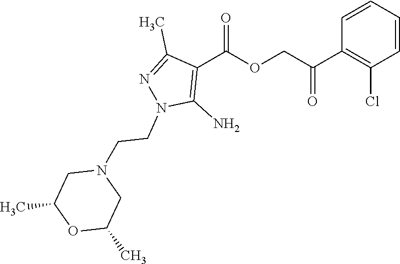 [SMILES: Cc1nn(CCN2C[C@@H](C)O[C@@H](C)C2)c(N)c1C(=O)OCC(=O)c1ccccc1Cl]

### Step 1: Ethyl 5-amino-1-(2-((2S ,6R)-2,6-dimethylmorpholino)ethyl)-3-methyl-1H-pyrazole-4-carboxylate

The titled compound was prepared by the reaction of ethyl (2E)-2-cyano-3-ethoxybut-2-enoate (3.70 g, 20.19 mmol) with (2S,6R)-4-(2-hydrazinylethyl)-2,6-dimethylmorpholine (4.2 g, 24.23 mmol) using N,N-Diisopropylethylamine (7 mL, 40.38 mmol) in dry ethanol (37 mL) as per the procedure described in Step 1 of Intermediate 75 to yield 3.0 g of the product as oil. ¹H NMR (300 MHz, DMSO-d₆): δ 1.03 (d, J=5.7 Hz, 6H), 1.24 (t, J=7.2 Hz, 3H), 1.66 (t, J=10.8 Hz, 2H), 2.14 (s, 3H), 2.50-2.58 (m, 2H), 2.75-2.83 (m, 2H), 3.42-3.49 (m, 2H), 3.92 (t, J=6.6 Hz, 2H), 4.14 (q, J=6.3 Hz, 2H), 6.25 (s, 2H).

### Step 2: 5-Amino-1-(2-((2S ,6R)-2,6-dimethylmorpholino)ethyl)-3-methyl-1H-pyrazole-4-carboxylic acid

The titled intermediate was prepared by the ester hydrolysis of Step 1 intermediate (2.9 g, 9.35 mmol) using aqueous solution of potassium hydroxide (2 M, 2.2 g, 39.28 mmol in 10 mL water) in ethanol (20 mL) as per the procedure described in Step 3 of Intermediate 1 to yield 1.75 g of the product as a sticky solid. ¹H NMR (300 MHz, DMSO-d₆): δ 1.01 (d, J=9.0 Hz, 6H), 1.58-1.78 (m, 2H), 2.11 (s, 3H), 2.43-2.50 (m, 2H), 2.72-2.83 (m, 2H), 3.42-3.59 (m, 2H), 3.82-3.96 (m, 2H), 6.19 (s, 2H); ESI (m/z) 281 (M−H)⁻

### Step 3: 2-(2-Chlorophenyl)-2-oxoethyl 5-amino-1-(2-((2S,6R)-2,6-dimethylmorpholino)ethyl)-3-methyl-1H-pyrazole-4-carboxylate

The titled compound was prepared by the reaction of Step 2 intermediate (1.70 g, 6.02 mmol) with 2-bromo-1-(2-chlorophenyl)ethanone (1.70 g, 7.23 mmol) using potassium fluoride (525 mg, 525 mmol) in dry DMF (15 mL) as per the procedure described in Step 4 of Intermediate 1 to yield 1.72 g of the product as sticky solid. ¹H NMR (300 MHz, DMSO-d₆): δ 1.03 (t, J=6.6 Hz, 6H), 1.65 (t, J=11.1 Hz, 2H), 2.13 (s, 3H), 2.49-2.2.60 (m, 2H), 2.73-2.84 (m, 2H), 3.43-3.3.59 (m, 2H), 3.89-3.99 (m, 2H), 5.30 (s, 2H), 6.36 (s, 2H), 7.44-7.60 (m, 3H), 7.77 (d, J=6.6 Hz, 1H).

### Intermediate 156

2-(2-Chlorophenyl)-2-oxoethyl 5-amino-3-methyl-1-(2-(piperidin-1-yl)ethyl)-1H-pyrazole-4-carboxylate

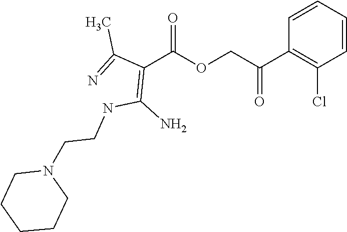 [SMILES: CC(=N)/C(C(=O)OCC(=O)c1ccccc1Cl)=C(/N)NCCN1CCCCC1]

### Step 1: Benzyl 5-amino-3-methyl-1-(2-(piperidin-1-yl)ethyl)-1H-pyrazole-4-carboxylate

The titled compound was prepared by the reaction of (E)-benzyl 2-cyano-3-ethoxybut-2-enoate (1.5 g, 6.12 mmol) with 1-(2-hydrazinylethyl)piperidine (1.1 g, 7.34 mmol) using N,N-Diisopropylethylamine (2.1 mL, 12.24 mmol) in dry ethanol (15 mL) as per the procedure described in Step 1 of Intermediate 75 to yield 1.05 g of the product as oil. ¹H NMR (300 MHz, DMSO-d₆): δ 1.30-1.57 (m, 6H), 2.13 (s, 3H), 2.26-2.44 (m, 4H), 3.30-3.42 (m, 2H), 3.89 (t, J=6.6 Hz, 2H), 5.18 (s, 2H), 6.35 (s, 2H), 7.24-7.42 (m, 5H).

### Step 2: 5-Amino-3-methyl-1-(2-(piperidin-1-yl)ethyl)-1H-pyrazole-4-carboxylic acid

The titled intermediate was prepared by deprotection of Step 1 intermediate (1.0 g, 2.92 mmol) using palladium on carbon (10%, 300 mg) in methanol (10 mL) as per the procedure described in Step 5 of Intermediate 87 to yield 740 mg of the product as a sticky solid. ¹H NMR (300 MHz, DMSO-d₆): ¹H NMR (300 MHz, DMSO-d₆): δ 1.68-1.85 (m, 6H), 2.16 (s, 3H), 2.80-3.00 (m, 2H), 3.30-3.50 (m, 2H), 4.32 (t, J=6.6 Hz, 2H), 6.39 (br s, 2H), 10.47 (br s, 1H).

### Step 3: 2-(2-Chlorophenyl)-2-oxoethyl 5-amino-3-methyl-1-(2-(piperidin-1-yl)ethyl)-1H-pyrazole-4-carboxylate

The titled compound was prepared by the reaction of Step 2 intermediate (730 mg, 2.89 mmol) with 2-bromo-1-(2-chlorophenyl)ethanone (812 mg, 3.47 mmol) using potassium fluoride (252 mg, 4.33 mmol) in dry DMF (8.0 mL) as per the procedure described in Step 4 of Intermediate 1 to yield 310 mg of the product as sticky oil. ¹H NMR (300 MHz, DMSO-d₆): δ 1.28-1.53 (m, 6H), 2.11 (s, 3H), 2.25-2.60 (m, 4H), 3.12-3.17 (m, 2H), 3.91 (t, J=6.6 Hz, 2H), 5.28 (s, 2H), 6.41 (br s, 2H), 7.42-7.59 (m, 3H), 7.80 (d, J=6.6 Hz, 1H); ESI (m/z) 405 (M+H)⁺.

### Intermediate 157

2-(2-Chlorophenyl)-2-oxoethyl 5-amino-1-(2-morpholinoethyl)-3-(trifluoromethyl)-1H-pyrazole-4-carboxylate

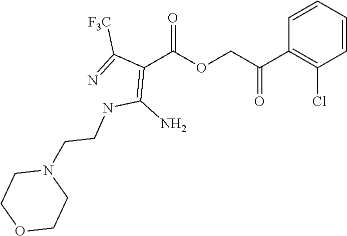 [SMILES: N=C(/C(C(=O)OCC(=O)c1ccccc1Cl)=C(/N)NCCN1CCOCC1)C(F)(F)F]

### Step 1: (E)-Ethyl 3-chloro-2-cyano-4,4,4-trifluorobut-2-enoate

The titled compound was prepared by the reaction of ethyl cyanoacetate (10.0 g, 88.40 mmol) with ethyl trifluoroacetate (15.5 g, 108.73 mmol) using sodium metal (2.1 g, 88.40 mmol) in dry ethanol (50 mL) as per the procedure described in Step 1 of Intermediate 39 followed by treating with phosphorus pentachloride (18.4 g, 88.40 mmol) in dichloromethane (100 mL) to give 5.0 g of the desired product as oil. ¹H NMR (300 MHz, CDCl₃): δ 1.33-1.49 (m, 3H). 4.37-4.53 (m, 2H).

### Step 2: Ethyl 5-amino-1-(2-morpholinoethyl)-3-(trifluoromethyl)-1H-pyrazole-4-carboxylate

The titled compound was prepared by the reaction of Step 1 intermediate (4.9 g, 21.53 mmol) with 4-(2-hydrazinylethyl)morpholine (3.5 g, 21.53 mmol) using triethylamine (7.6 mL, 53.84 mmol) in dry ethanol (50 mL) as per the procedure described in Step 1 of Intermediate 75 to yield 1.35 g of the product as oil. ¹H NMR (300 MHz, DMSO-d₆): δ 1.34 (t, J=7.5 Hz, 3H), 2.57-2.68 (m, 4H), 2.73-2.81 (m, 2H), 3.68-3.97 (m, 4H), 4.14-4.20 (m, 2H), 4.30 (q, J=6.9 Hz, 2H), 6.65 (br s, 2H).

### Step 3: 5-Amino-1-(2-morpholinoethyl)-3-(trifluoromethyl)-1H-pyrazole-4-carboxylic acid

The titled intermediate was prepared by the ester hydrolysis of Step 2 intermediate (1.30 g, 3.86 mmol) using aqueous solution of potassium hydroxide (2 M, 0.440 g, 7.73 mmol in 6.5 mL water) in ethanol (10 mL) as per the procedure described in Step 3 of Intermediate 1 to yield 960 mg of the product as a white solid. ¹H NMR (300 MHz, DMSO-d₆): δ 2.47-2.82 (m, 6H), 3.55-3.64 (m, 4H), 4.14 (t, J=6.6 Hz, 2H), 6.70 (br s, 2H), 12.12-13.28 (m, 1H); ESI (m/z) 309 (M+H)⁺.

### Step 4: 2-(2-Chlorophenyl)-2-oxoethyl 5-amino-1-(2-morpholinoethyl)-3-(trifluoromethyl)-1H-pyrazole-4-carboxylate

The titled compound was prepared by the reaction of Step 3 intermediate (940 mg, 3.04 mmol) with 2-bromo-1-(2-chlorophenyl)ethanone (790 mg, 3.35 mmol) using potassium fluoride (265 mg, 4.57 mmol) in dry DMF (9.5 mL) as per the procedure described in Step 4 of Intermediate 1 to yield 720 mg of the product as a sticky oil. ¹H NMR (300 MHz, DMSO-d₆): δ 2.60-2.74 (m, 4H), 2.78-2.85 (m, 2H), 3.70-3.80 (m, 4H), 4.17-4.24 (sm 2H), 5.38 (s, 2H), 6.80 (br s, 2H), 7.36-7.48 (m, 3H), 7.66 (d, J=6.6 Hz, 1H).

### Intermediate 158

2-(2-Chlorophenyl)-2-oxoethyl 5-amino-3-methyl-1-(2-(pyrrolidin-1-yl)ethyl)-1H-pyrazole-4-carboxylate

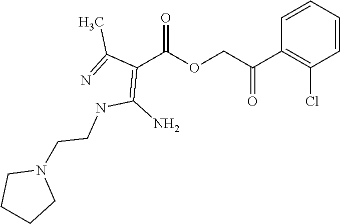 [SMILES: CC(=N)/C(C(=O)OCC(=O)c1ccccc1Cl)=C(/N)NCCN1CCCC1]

### Step 1: Benzyl 5-amino-3-methyl-1-(2-(pyrrolidin-1-yl)ethyl)-1H-pyrazole-4-carboxylate

The titled compound was prepared by the reaction of (E)-benzyl 2-cyano-3-ethoxybut-2-enoate (4.0 g, 16.32 mmol) with 1-(2-hydrazinylethyl)pyrrolidine (2.52 g, 19.59 mmol) using N,N-Diisopropylethylamine (5.58 mL, 32.65 mmol) in ethanol (40 mL) as perthe procedure described in Step 1 of Intermediate 75 to yield 1.95 g of the product. ¹H NMR (300 MHz, DMSO-d₆): δ 1.60-1.75 (m, 4H), 2.14 (s, 3H), 2.44-2.58 (m, 4H), 2.70-2.79 (m, 2H), 3.92 (t, J=6.6 Hz, 2H), 5.19 (s, 2H), 6.30 (s, 2H), 7.24-7.40 (m, 5H).

### Step 2: 5-Amino-3-methyl-1-(2-(pyrrolidin-1-yl)ethyl)-1H-pyrazole-4-carboxylic acid

The titled intermediate was prepared by the deprotection of of Step 1 intermediate (1.90 g, 5.79 mmol) using palladium on carbon (10%, 600 mg) in methanol (20 mL) as per the procedure described in Step 5 of Intermediate 87 to yield 1.21 g of the product as a solid. ¹H NMR (300 MHz, DMSO-d₆): δ 1.84-1.93 (m, 4H), 2.17 (s, 3H), 2.44-2.56 (m, 6H), 4.17-4.24 (m, 2H), 6.37 (s, 2H).

### Step 3: 2-(2-Chlorophenyl)-2-oxoethyl 5-amino-3-methyl-1-(2-(pyrrolidin-1-yl)ethyl)-1H-pyrazole-4-carboxylate

The titled compound was prepared by the reaction of Step 2 intermediate (600 mg, 2.52 mmol) with 2-bromo-1-(2-chlorophenyl)ethanone (653 mg, 2.77 mmol) using potassium fluoride (219 mg, 3.78 mmol) in dry DMF (6.0 mL) as per the procedure described in Step 4 of Intermediate 1 to yield 289 mg of the product. ¹H NMR (300 MHz, DMSO-d₆): δ 1.63-1.75 (m, 4H), 2.11 (s, 3H), 2.40-2.64 (m, 4H), 2.67-2.76 (m, 2H), 3.89-4.00 (m, 2H), 5.29 (s, 2H), 6.36 (s, 2H), 7.45-7.59 (m, 2H), 7.73 (d, J=6.9 Hz, 1H).

### Intermediate 159

2-Oxo-2-(2-(trifluoromethyl)phenyl)ethyl 5-amino-3-methyl-1-(2-morpholinoethyl)-1H-pyrazole-4-carboxylate

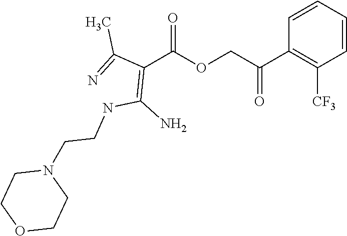 [SMILES: CC(=N)/C(C(=O)OCC(=O)c1ccccc1C(F)(F)F)=C(/N)NCCN1CCOCC1]

The titled compound was prepared by the reaction of Step 2 of Intermediate 91 (750 mg, 2.95 mmol) with 2-bromo-1-(2-(trifluoromethyl)phenyl)ethanone (768 mg, 3.24 mmol) using potassium fluoride (257 mg, 4.42 mmol) in dry DMF (8.0 mL) as per the procedure described in Step 4 of Intermediate 1 to yield 940 mg of the product as a sticky solid. ¹H NMR (300 MHz, DMSO-d₆): δ 2.10 (s, 3H), 2.40-2.64 (m, 6H), 3.52-3.60 (m, 4H), 3.94 (t, J=6.6 Hz, 2H), 5.31 (s, 2H), 6.38 (s, 2H), 7.78-7.95 (m, 4H).

### Intermediate 160

2-(2-Fluoro-3-(trifluoromethyl)phenyl)-2-oxoethyl 5-amino-3-methyl-1-(2-morpholinoethyl)-1H-pyrazole-4-carboxylate

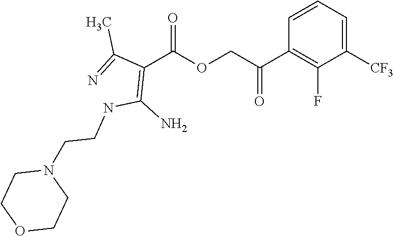 [SMILES: CC(=N)/C(C(=O)OCC(=O)c1cccc(C(F)(F)F)c1F)=C(/N)NCCN1CCOCC1]

The titled compound was prepared by the reaction of 5-amino-3-methyl-1-(2-morpholinoethyl)-1H-pyrazole-4-carboxylic acid Step 2 Intermediate 91 (800 mg, 3.14 mmol) with 2-bromo-1-(2-fluoro-3-(trifluoromethyl)phenyl)ethanone (987 mg, 3.46 mmol) using potassium fluoride (274 mg, 4.72 mmol) in dry DMF (8.0 mL) as per the procedure described in Step 4 of Intermediate 1 to yield 1.01 g of the product as a solid. ¹H NMR (300 MHz, DMSO-d₆): δ 2.17 (s, 3H), 2.40-2.56 (m, 4H), 2.59 (t, J=6.8 Hz, 2H), 3.50-3.60 (m, 4H), 3.95 (t, J=6.6 Hz, 2H), 5.38 (s, 2H), 6.38 (s, 2H), 7.60 (t, J=8.1 Hz, 1H), 8.09 (t, J=7.2 Hz, 1H), 8.19 (t, J=6.9 Hz, 1H).

### Intermediate 161

2-(2-Chlorophenyl)-2-oxoethyl 5-amino-1-(2-(dimethylamino)ethyl)-3-(trifluoromethyl)-1H-pyrazole-4-carboxylate

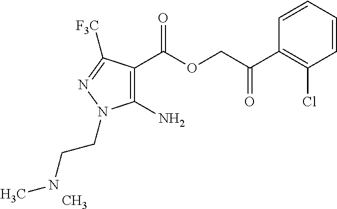

### Step 1: Ethyl 5-amino-1-(2-(dimethylamino)ethyl)-3-(trifluoromethyl)-1H-pyrazole-4-carboxylate

The titled compound was prepared by the reaction of ethyl (E)-ethyl 3-chloro-2-cyano-4,4,4-trifluorobut-2-enoate (10 g, 40.98 mmol) with 2-hydrazinyl-N,N-dimethylethanamine (4.3 g, 40.98 mmol) using triethyl amine (11.5 mL, 81.96 mmol) in dry methanol (100 mL) as per the procedure described in Step 1 of Intermediate 75 to yield 1.68 g of the product as oil. ¹H NMR (300 MHz, DMSO-d₆): δ 1.23 (t, J=7.2 Hz, 3H), 2.17 (s, 6H), 2.57 (t, J=6.3 Hz, 2H), 4.07 (t, J=6.3 Hz, 2H), 4.18 (q, J=7.2 Hz, 2H), 6.72 (s, 2H).

### Step 2: 5-Amino-1-(2-(dimethylamino)ethyl)-3-(trifluoromethyl)-1H-pyrazole-4-carboxylic acid

The titled intermediate was prepared by the ester hydrolysis of Step 1 intermediate (1.6 g, 5.44 mmol) using aqueous solution of potassium hydroxide (609 mg, 10.88 mmol in 6.0 mL of water) in ethanol (16 mL) as per the procedure described in Step 3 of Intermediate 1 to yield 745 mg of the product as a white solid. ¹H NMR (300 MHz, DMSO-d₆): δ 2.18 (s, 6H), 2.58 (t, J=6.3 Hz, 3H), 4.06 (t, J=6.6 Hz, 2H), 6.65 (br s, 2H); APCI (m/z) 267 (M+H)⁺.

### Step 3: 2-(2-Chlorophenyl)-2-oxoethyl 5-amino-1-(2-(dimethylamino)ethyl)-3-(trifluoromethyl)-1H-pyrazole-4-carboxylate

The titled compound was prepared by the reaction of Step 2 intermediate (735 mg, 2.76 mmol) with 2-bromo-1-(2-chlorophenyl)ethanone (645 mg, 2.76 mmol) using potassium fluoride (240 mg, 4.14 mmol) in dry DMF (7.0 mL) as per the procedure described in Step 4 of Intermediate 1 to yield 533 mg of the product as oil. ¹H NMR (300 MHz, DMSO-d₆): δ 2.19 (s, 6H), 2.60 (t, J=6.6 Hz, 2H), 4.10 (t, J=6.6 Hz, 2H), 5.38 (s, 2H), 6.85 (s, 2H), 7.47-7.55 (m, 1H), 7.59 (d, J=3.9 Hz, 2H), 7.80 (d, J=7.2 Hz, 1H).

### Intermediate 162

2-(2-Chlorophenyl)-2-oxoethyl 5-amino-3-methyl-1-(2-(4-methylpiperazin-1-yl)ethyl)-1H-pyrazole-4-carboxylate

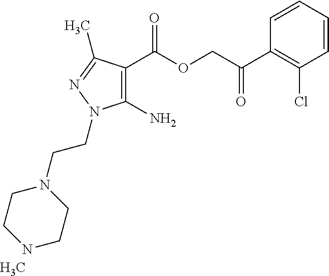

### Step 1: Ethyl 5-amino -3-methyl-1-(2-(4-methylpiperazin-1-yl)ethyl)-1H-pyrazole-4-carboxylate

The titled compound was prepared by the reaction of ethyl (2E)-2-cyano-3-ethoxybut-2-enoate (1.50 g, 8.17 mmol) with 1-(2-hydrazinylethyl)-4-methylpiperazine (1.55 g, 9.80 mmol) using N,N-Diisopropylethylamine (2.80 mL, 16.34 mmol) in ethanol (15 mL) as per the procedure described in Step 1 of Intermediate 75 to yield 1.5 g of the product as oil. ¹H NMR (300 MHz, CDCl₃): δ 1.35 (t, J=6.9 Hz, 3H), 2.31 (s, 6H), 2.35-2.80 (m, 10H), 4.02 (t, J=5.1 Hz, 2H), 4.27 (q, J=6.9 Hz, 2H), 6.30 (s, 2H).

### Step 2: 5-Amino-3-methyl-1-(2-(4-methylpiperazin-1-yl)ethyl)-1H-pyrazole-4-carboxylic acid

The titled intermediate was prepared by the ester hydrolysis of Step 1 intermediate (1.45 g, 4.90 mmol) using aqueous solution of potassium hydroxide (550 mg, 9.8 mmol in 6 mL water) in IPA (15 mL) as per the procedure described in Step 3 of Intermediate 1. The solvent was then evaporated under reduced pressure followed by co-distilation with isopropanol (3×75 mL) yielded crude product which was used directly for the next step.

### Step 3: 2-(2-Chlorophenyl)-2-oxoethyl 5-amino-3-methyl-1-(2-(4-methylpiperazin-1-yl)ethyl)-1H-pyrazole-4-carboxylate

The titled compound was prepared by the reaction of Step 2 intermediate (1.20 g, 4.4 mmol) with 2-bromo-1-(2-chlorophenyl)ethanone (1.11 g, 4.93 mmol) using potassium fluoride (390 mg, 6.73 mmol) in dry DMF (12.0 mL) as per the procedure described in Step 4 of Intermediate 1 to yield 910 mg of the product as white solid. ¹H NMR (300 MHz, CDCl₃): δ 2.27 (s, 3H), 2.33 (s, 3H), 2.40-2.77 (m, 10H), 4.00-4.08 (m, 2H), 5.32 (s, 2H), 6.37 (br s, 2H), 7.35-7.42 (m, 1H), 7.44 (d, J=3.3 Hz, 2H), 7.65 (d, J=6.9 Hz, 1H).

### Intermediate 163

3-(2,6-Difluorophenyl)-2-oxopropyl 5-amino-3-ethyl-1-(2-morpholinoethyl)-1H-pyrazole-4-carboxylate

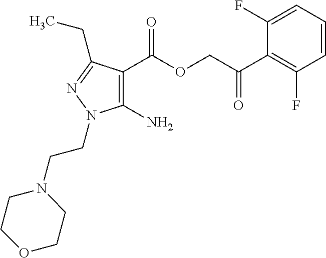 [SMILES: CCc1nn(CCN2CCOCC2)c(N)c1C(=O)OCC(=O)c1c(F)cccc1F]

The titled compound was prepared by the reaction of Step 2 intermediate 153 (1.9 g, 6.41 mmol) with 2-bromo-1-(2,6-difluorophenyl)ethanone (1.51 g, 6.41 mmol) using potassium fluoride (560 mg, 9.62 mmol) in dry DMF (20.0 mL) as per the procedure described in Step 4 of Intermediate 1 to yield 1.94 g of the product. ¹H NMR (300 MHz, DMSO-d₆): δ 1.07 (t, J=7.2 Hz, 3H), 2.38-2.63 (m, 8H), 3.51-3.60 (m, 4H), 3.94-4.00 (m, 2H), 5.19 (s, 2H), 6.38 (s, 2H), 7.27 (t, J=8.7 Hz, 2H), 7.60-7.69 (m, 1H).

### Intermediate 164

2-(2,6-Difluorophenyl)-2-oxoethyl 1-(2-(1H-pyrazol-1-yl)ethyl)-5-amino-3-ethyl-1H-pyrazole-4-carboxylate

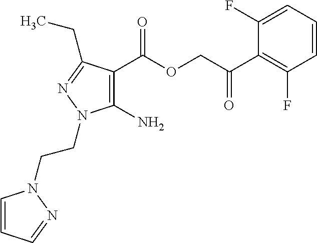 [SMILES: CCc1nn(CCn2cccn2)c(N)c1C(=O)OCC(=O)c1c(F)cccc1F]

### Step 1: Ethyl 1-(2-(1H-pyrazol-1-yl)ethyl)-5-amino-3-ethyl-1H-pyrazole-4-carboxylate

The titled compound was prepared by the reaction of (E)-ethyl 2-cyano-3-ethoxypent-2-enoate (4.0 g, 20.30 mmol) with 1-(2-hydrazinylethyl)-1H-pyrazole (3.0g, 23.77 mmol) using N,N-diisopropylethylamine (6.94, 40.59 mmol) in IPA (40 mL) as per the procedure described in Step 1 of Intermediate 75 to yield 2.88 g of the product as oil. ¹H NMR (300 MHz, DMSO-d₆): δ 1.09 (t, J=7.5 Hz, 3H), 1.23 (t, J=6.9 Hz, 3H), 2.59 (q, J=7.8 Hz, 2H), 4.14 (q, J=7.2 Hz, 2H), 4.19-4.28 (m, 2H), 4.37-4.44 (m, 2H), 6.11 (s, 2H), 6.19 (s, 1H), 7.44 (s, 1H), 7.51 (s, 1H)

### Step 2: 1-(2-(1H-Pyrazol-1-yl)ethyl)-5-amino-3-ethyl-1H-pyrazole-4-carbo xylic acid

The titled intermediate was prepared by the ester hydrolysis of Step 1 intermediate (2.8 g, 10.63 mmol) using aqueous solution of potassium hydroxide (2 M, 1.19 g, 21.25 mmol in 12 mL water) in IPA (28 mL) as per the procedure described in Step 3 of Intermediate 1 to yield 2.0 g of the product as a solid. ¹H NMR (300 MHz, DMSO-d₆): δ 1.09 (t, J=7.5 Hz, 2H), 2.58 (t, J=7.2 Hz, 3H), 4.21 (t, J=6.3 Hz, 2H), 4.41 (t, J=6.0 Hz, 2H), 6.10 (br, s, 2H), 6.19 (s, 1H), 7.44 (s, 1H), 7.51 (s, 1H).

### Step 3: 2-(2,6-Difluorophenyl)-2-oxoethyl 1-(2-(1H-pyrazol-1-yl)ethyl)-5-amino-3-ethyl-1H-pyrazole-4-carboxylate

The titled compound was prepared by the reaction of Step 2 intermediate (1.00 g, 4.01 mmol) with 2-bromo-1-(2,6-difluorophenyl)ethanone (940 mg, 4.01 mmol) using potassium fluoride (350 mg, 6.03 mmol) in dry DMF (10 mL) as per the procedure described in Step 4 of Intermediate 1 to yield 1.19 g of the product . ¹H NMR (300 MHz, DMSO-d₆): δ 1.07 (t, J=7.2 Hz, 3H), 2.47-2.60 (m, 2H), 4.23 (t, J=6.3 Hz, 2H), 4.42 (t, J=6.8 Hz, 2H), 5.18 (s, 2H), 6.15-6.25 (m, 3H), 7.25 (t, J=8.7 Hz, 2H), 7.44 (s, 1H), 7.52 (s, 1H), 7.65-7.70 (m, 1H).

### Intermediate 165

2-(2-Chlorophenyl)-2-oxoethyl 1-(2-(1H-pyrazol-1-yl)ethyl)-5-amino-3-ethyl-1H-pyrazole-4-carboxylate

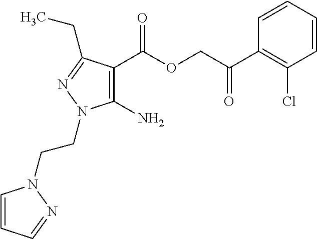 [SMILES: CCc1nn(CCn2cccn2)c(N)c1C(=O)OCC(=O)c1ccccc1Cl]

The titled compound was prepared by the reaction of Step 2 of intermediate 164 (1.0 g,4.01 mmol) with 2-bromo-1-(2-chlorophenyl)ethanone (936 mg, 4.00 mmol) using potassium fluoride (350 mg, 6.02 mmol) in dry DMF (10 mL) as per the procedure described in Step 4 of Intermediate 1 to yield 1.41 g of the product as solid. ¹H NMR (300 MHz, DMSO-d₆): δ 1.07 (t, J=7.2 Hz, 3H), 2.57 (q, J=7.8 Hz, 2H), 4.20-4.37 (m, 2H), 4.39-4.45 (m, 2H), 5.30 (s, 2H), 6.20 (br, s, 2H), 6.24 (s, 1H), 7.44-7.62 (m, 5H), 7.77 (d, J=7.2 Hz, 1H).

### Intermediate 166

2-(4-Fluoro-3-(trifluoromethyl)phenyl)-2-oxoethyl 5-amino-3-ethyl-1-methyl-1H-pyrazole-4-carboxylate

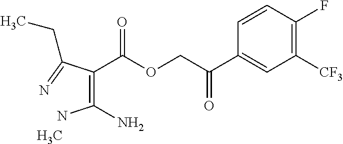

The titled compound was prepared by the reaction of Step 2 of intermediate 75 (1.0 g, 5.9 mmol) with 2-bromo-1-(4-fluoro-3-(trifluoromethyl)phenyl)ethanone (1.85 g, 6.5 mmol) using potassium fluoride (520mg, 8.8 mmol) in dry DMF (10 mL) as per the procedure described in Step 4 of Intermediate 1 to yield 1.30 g of the product as sticky solid. ¹H NMR (300 MHz, DMSO-d₆): δ 1.11 (t, J=7.2 Hz, 3H), 2.61 (q, J=7.2 Hz, 2H), 3.49 (s, 3H), 5.58 (s, 2H), 6.27 (s, 2H), 7.74 (t, J=9.6 Hz, 1H), 8.31 (d, J=6.9 Hz, 1H), 8.36-8.44 (m, 1H)

### Intermediate 167

2-(2-Fluoro-3-(trifluoromethyl)phenyl)-2-oxoethyl 5-amino-3-ethyl-1-methyl-1H-pyrazole-4-carboxylate

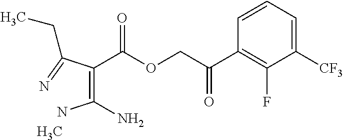

The titled compound was prepared by the reaction of Step 2 of intermediate 75 (850 mg, 5.02 mmol) with 2-bromo-1-(2-fluoro-3-(trifluoromethyl)phenyl)ethanone (1.6 g, 5.53 mmol) using potassium fluoride (440 mg, 7.54 mmol) in dry DMF (15 mL) as per the procedure described in Step 4 of Intermediate 1 to yield 1.15 g of the product as white solid. ¹H NMR (300 MHz, DMSO-d₆): δ 1.09 (t, J=7.5 Hz, 3H), 2.60 (q, J=7.2 Hz, 2H), 3.48 (s, 3H), 5.38 (s, 2H), 6.27 (s, 2H), 7.58 (t, J=6.9 Hz, 1H), 8.09 (t, J=7.2 Hz, 1H), 8.19 (t, J=6.9 Hz, 1H).

### Intermediate 168

2-(2,4-Difluorophenyl)-2-oxoethyl 5-amino-3-ethyl-1-methyl-1H-pyrazole-4-carboxylate

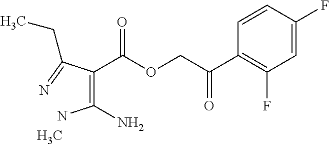

The titled compound was prepared by the reaction of Step 2 of intermediate 75 (1.00 g, 5.91 mmol) with 2-bromo-1-(2,4-difluorophenyl)ethanone (1.40 g, 5.91 mmol) using potassium fluoride (515 mg, 8.87 mmol) in dry DMF (15 mL) as per the procedure described in Step 4 of Intermediate 1 to yield 1.45 g of the product as a solid. ¹H NMR (300 MHz, DMSO-d₆): δ 1.10 (t, J=7.2 Hz, 3H), 2.61 (q, J=6.3 Hz, 2H), 3.48 (s, 3H), 5.33 (s, 2H), 6.26 (s, 2H), 7.29 (t, J=7.2 Hz, 1H), 7.50 (t, J=6.9 Hz, 1H), 7.98 (t, J=7.2 Hz, 1H).

### Intermediate 169

2-(3,5-Difluorophenyl)-2-oxoethyl 5-amino-3-ethyl-1-methyl-1H-pyrazole-4-carboxylate

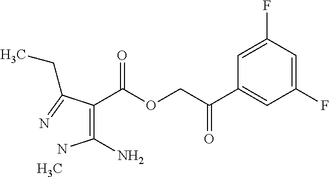

The titled compound was prepared by the reaction of Step 2 intermediate 75 (1.0 g, 5.91 mmol) with 2-bromo-1-(3,5-difluorophenyl)ethanone (1.40 g, 5.91 mmol) using potassium fluoride (525 mg, 8.87 mmol) in dry DMF (10 mL) as per the procedure described in Step 4 of Intermediate 1 to yield 1.06 g of the product as a solid. ¹H NMR (300 MHz, DMSO-d₆): δ 1.09 (t, J=7.5 Hz, 3H), 2.59 (t, J=7.5 Hz, 2H), 3.47 (s, 3H), 5.50 (s, 2H), 6.25 (s, 2H), 7.58-7.76 (m, 3H); ESI (m/z) 324 (M+H).

### Intermediate 170

2-(2,5-Difluorophenyl)-2-oxoethyl 5-amino-3-ethyl-1-methyl-1H-pyrazole-4-carboxylate

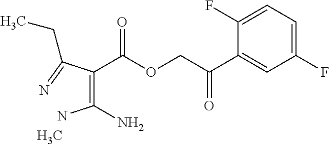

The titled compound was prepared by the reaction of Step 2 intermediate 75 (1.0 g, 5.91 mmol) with 2-bromo-1-(2,5-difluorophenyl)ethanone (1.40 g, 5.91 mmol) using potassium fluoride (525 mg, 8.87 mmol) in dry DMF (10 mL) as per the procedure described in Step 4 of Intermediate 1 to yield 420 mg of the product as a solid. ¹H NMR (300 MHz, DMSO-d₆): δ 1.10 (t, J=7.8 Hz, 3H), 2.61 (q, J=7.2 Hz, 2H), 3.48 (s, 3H), 5.35 (s, 2H), 6.27 (br s, 2H), 7.40-7.75 (m, 3H).

### Intermediate 171

2-(2-Fluoro-3-(trifluoromethyl)phenyl)-2-oxoethyl 5-amino-3-ethyl-1-(2-morpholinoethyl)-1H-pyrazole-4-carboxylate

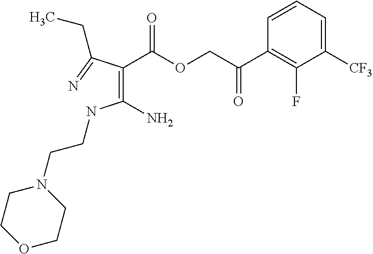 [SMILES: CCC(=N)/C(C(=O)OCC(=O)c1cccc(C(F)(F)F)c1F)=C(/N)NCCN1CCOCC1]

The titled compound was prepared by the reaction of Step 2 of intermediate 153 (1.75 g, 6.52 mmol) with 2-bromo-1-(2-fluoro-3-(trifluoromethyl)phenyl)ethanone (2.04 g, 7.18 mmol) using potassium fluoride (569 mg, 9.79 mmol) in dry DMF (18 mL) as per the procedure described in Step 4 of Intermediate 1 to yield 1.54 g of the product . ¹H NMR (300 MHz, DMSO-d₆): δ 1.09 (t, J=6.9 Hz, 3H), 2.38-2.49 (m, 4H), 2.57-2.64 (m, 4H), 3.52-3.64 (m, 4H), 3.97 (t, J=6.9 Hz, 2H), 5.38 (s, 2H), 6.39 (s, 2H), 7.57 (t, J=7.2 Hz, 1H), 8.09 (t, J=6.9 Hz, 1H), 8.20 (t, J=7.2 Hz, 1H).

### Intermediate 172

2-(2-Chlorophenyl)-2-oxoethyl 5-amino-3-ethyl-1-(2-(4-methylpiperazin-1-yl)ethyl)-1H-pyrazole-4-carboxylate

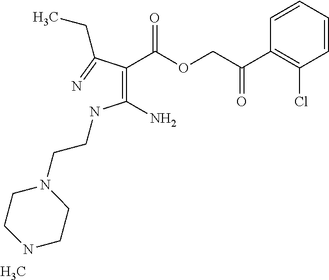

### Step 1: Ethyl 5-amino-3-ethyl-1-(2-(4-methylpiperazin-1-yl)ethyl)-1H-pyrazole-4-carboxylate

The titled compound was prepared by the reaction of ethyl 2-cyano-3-ethoxypent-2-enoate (4.0 g, 20.28 mmol) with 1-(2-hydrazinylethyl)-4-methylpiperazine (3.85 g, 24.30 mmol) using N,N-Diisopropylethylamine (7.0 mL, 4056 mmol) in ethanol (40 mL) as per the procedure described in Step 1 of Intermediate 75 to yield 1.65 g of the product as sticky oil. ¹H NMR (300 MHz, DMSO-d₆): δ 1.08 (t, J=7.2 Hz, 3H), 1.24 (t, J=6.9 Hz, 3H), 2.19 (s, 3H), 2.24-2.64 (m, 12H), 3.92 (t, J=6.9 Hz, 2H), 4.14 (q, J=7.2 Hz, 2H), 6.26 (s, 2H).

### Step 2: 5-amino-3-ethyl-1-(2-(4-methylpiperazin-1-yl)ethyl)-1H-pyrazole-4-carboxylic acid

The titled intermediate was prepared by the ester hydrolysis of Step 1 intermediate (2.10 g, 6.78 mmol) using aqueous solution of potassium hydroxide (760 mg, 13.58 mmol in 2.5mL water) in EtOH (15 mL) as per the procedure described in Step 3 of Intermediate 1 The solvent was then evaporated under reduced pressure followed by co-distilation with isopropanol (3×75 ml) yielded the crude product, which was used directly for the next step.

### Step 3: 2-(2-Chlorophenyl)-2-oxoethyl 5-amino-3-ethyl-1-(2-(4-methylpiperazin-1-yl)ethyl)-1H-pyrazole-4-carboxylate

The titled compound was prepared by the reaction of Step 2 intermediate (1.65 g, 5.86mmol) with 2-bromo-1-(2-chlorophenyl)ethanone (1.50 g, 6.44 mmol) using potassium fluoride (510 mg, 8.80 mmol) in dry DMF (16.0 mL) as per the procedure described in Step 4 of Intermediate 1 to yield 1.30g of the product as a sticky oil . ¹H NMR (300 MHz, DMSO-d₆): δ 1.07 (t, J=7.2 Hz, 3H), 2.17 (s, 3H), 2.23-2.64 (m, 12H), 3.94 (t, J=6.6 Hz, 2H), 5.31 (s, 2H), 6.39 (br s, 2H), 7.45-7.55 (m, 1H), 7.58 (d, J=7.8 Hz, 2H), 7.78 (d, J=8.4 Hz, 1H); ESI (m/z) 434 (M+H)⁺.

### Intermediate 173

2-(2,6-Difluorophenyl)-2-oxoethyl 5-amino-3-ethyl-1-(2-(4-methylpiperazin-1-yl)ethyl)-1H-pyrazole-4-carboxylate

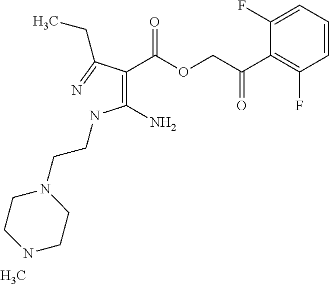

The titled compound was prepared by the reaction of Step 2 intermediate 172 (2.0 g, 7.10 mmol) with 2-bromo-1-(2,6-difluorophenyl)ethanone (1.67 g, 7.10 mmol) using potassium fluoride (620 mg, 10.67 mmol) in dry DMF (20.0mL) as per the procedure described in Step 4 of Intermediate 1 to yield 1.10 g of the product. ¹H NMR (300 MHz, DMSO-d₆): δ 1.06 (t, J=7.2 Hz, 3H), 2.19 (s, 3H), 2.24-2.64 (m, 12H), 3.92-4.04 (m, 2H), 5.19 (s, 2H), 6.39 (br s, 2H), 7.27 (d, J=9.0 Hz, 2H), 7.64-7.69 (m, 1H).

### Intermediate 174

2-(2-Chlorophenyl)-2-oxoethyl 5-amino-3-ethyl-1-(2-(3-oxomorpholino)ethyl)-1H-pyrazole-4-carboxylate

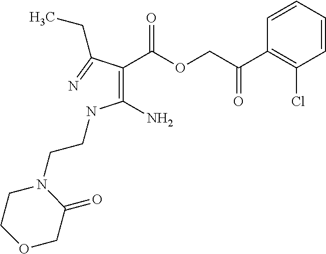 [SMILES: CCC(=N)/C(C(=O)OCC(=O)c1ccccc1Cl)=C(/N)NCCN1CCOCC1=O]

### Step 1: Ethyl 5-amino-3-ethyl-1-(2-(3-oxomorpholino)ethyl)-1H-pyrazole-4-carboxylate

The titled compound was prepared by the reaction of (E)-ethyl 2-cyano-3-ethoxypent-2-enoate (1.30 g, 6.68 mmol) with 4-(2-hydrazinylethyl)morpholin-3-one (2.50 g, 6.68 mmol) using N,N-Diisopropylethylamine (2.30 mL, 13.36 mmol) in ethanol (25 mL) as per the procedure described in Step 1 of Intermediate 75 to yield 1.10 g of the product as oil. ¹H NMR (300 MHz, DMSO-d₆): δ 1.09 (t, J=7.2 Hz, 3H), 1.24 (t, J=6.9 Hz, 3H), 2.60 (q, J=7.2 Hz, 2H), 3.10-3.18 (m, 2H), 3.54 (t, J=6.6 Hz, 2H), 3.72 (t, J=6.6 Hz, 2H), 3.96 (s, 2H), 4.02 (q, J=6.3 Hz, 2H), 4.15 (q, J=7.2 Hz, 2H), 6.22 (s, 2H).

### Step 2: 5-Amino-3-ethyl-1-(2-(3-oxomorpholino)ethyl)-1H-pyrazole-4-carboxylic acid

The titled intermediate was prepared by the ester hydrolysis of Step 1 intermediate (1.50 g, 4.85 mmol) using aqueous solution of potassium hydroxide (543 mg, 9.70 mmol in 5.5mL water) in EtOH (10 mL) as per the procedure described in Step 3 of Intermediate 1. The solvent was then evaporated under reduced pressure and lypholized or co-distilled with isopropanol (3×75 ml) to obtain the crude product, obtained was carried forward for the next reaction.

### Step 3: 2-(2-Chlorophenyl)-2-oxoethyl 5-amino-3-ethyl-1-(2-(3-oxomorpholino)ethyl)-1H-pyrazole-4-carboxylate

The titled compound was prepared by the reaction of Step 2 intermediate (1.20 g, 4.27 mmol) with 2-bromo-1-(2-chlorophenyl)ethanone (1.20 g, 5.12 mmol) using potassium fluoride (372 mg, 6.40 mmol) in dry DMF (12 mL) as per the procedure described in Step 4 of Intermediate 1 to yield 560 mg of the product as oil. ¹H NMR (300 MHz, DMSO-d₆): δ 1.07 (t, J=7.5 Hz, 3H), 2.55 (q, J=7.2 Hz, 2H), 3.10-3.20 (m, 2H), 3.55 (t, J=6.6 Hz, 2H), 3.72 (t, J=6.6 Hz, 2H), 3.97 (s, 2H), 4.04 (t, J=6.8 Hz, 2H), 5.31 (s, 2H), 6.35 (s, 2H), 7.48-7.59 (m, 2H), 7.78 (d, J=7.2 Hz, 1H), 7.95 (s, 1H); ESI (m/z) 434 (M+H)⁺.

### Intermediate 175

2-(2,6-Difluorophenyl)-2-oxoethyl 5-amino-3-ethyl-1-(2-(3-oxomorpholino)ethyl)-1H-pyrazole-4-carboxylate

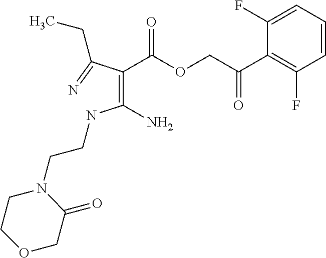 [SMILES: CCC(=N)/C(C(=O)OCC(=O)c1c(F)cccc1F)=C(/N)NCCN1CCOCC1=O]

The titled compound was prepared by the reaction of Step 2 intermediate of 174 (1.5 g, 5.33 mmol) with 2-bromo-1-(2,6-difluorophenyl)ethanone (1.4 g, 5.87 mmol) using potassium fluoride (463 mg, 7.99 mmol) in dry DMF (15 mL) as per the procedure described in Step 4 of Intermediate 1 to yield 240 mg of the product as sticky oil. ¹H NMR (300 MHz, DMSO-d₆): δ 1.07 (t, J=7.5 Hz, 3H), 2.55 (q, J=7.2 Hz, 2H), 3.10-3.20 (m, 2H), 3.53 (t, J=6.6 Hz, 2H), 3.71 (t, J=6.6 Hz, 2H), 3.95 (s, 2H), 4.01 (t, J=6.8 Hz, 2H), 5.17 (s, 2H), 6.32 (s, 2H), 7.26 (t, J=8.7 Hz, 2H), 7.62-7.69 (m, 1H).

### Intermediate 176

2-(2,6-Difluorophenyl)-2-oxoethyl 5-amino-3-methyl-1-(2-(4-methylpiperazin-1-yl)ethyl)-1H-pyrazole-4-carboxylate

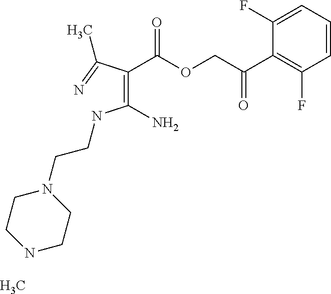

The titled compound was prepared by the reaction of Step 2 intermediate 162 (1.0 g, 3.74 mmol) with 2-bromo-1-(2,6-difluorophenyl)ethanone (965 mg, 4.11 mmol) using potassium fluoride (330 mg, 5.61 mmol) in dry DMF (10 mL) as per the procedure described in Step 4 of Intermediate 1 to yield 770 mg of the product as sticky oil. ¹H NMR (500 MHz, DMSO-d₆): δ 2.11 (s, 3H), 2.16 (s, 3H), 2.29-2.31 (m, 4H), 2.40-2.54 (m, 4H), 2.57 (t, J=7.0 Hz, 2H), 3.92 (t, J=6.5 Hz, 2H), 5.19 (s, 2H), 6.38 (s, 2H), 7.26 (t, J=8.5 Hz, 2H), 7.64-7.71 (m, 1H); ESI (m/z) 421.96 (M+H)⁺.

### Intermediate 177

2-(2,4-Difluorophenyl)-2-oxoethyl 5-amino-3-ethyl-1-propyl-1H-pyrazole-4-carboxylate

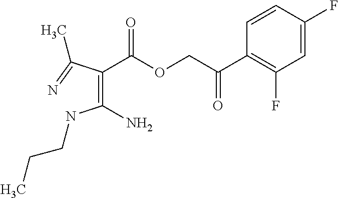 [SMILES: CCCN/C(N)=C(\C(C)=N)C(=O)OCC(=O)c1ccc(F)cc1F]

### Step 1: Ethyl 5-amino-3-ethyl-1-propyl-1H-pyrazole-4-carboxylate

The titled compound was prepared by the reaction of (E)-ethyl 2-cyano-3-ethoxypent-2-enoate (25.0 g, 127 mmol) with propylhydrazine (9.5 g, 127 mmol) using N,N-Diisopropylethylamine (44 mL, 245 mmol) in ethanol (250 mL) as per the procedure described in Step 1 of Intermediate 75 to yield 19.0 g of the product as oil. ¹H NMR (300 MHz, DMSO-d₆): δ 0.80 (t, J=7.2 Hz, 3H), 1.07 (t, J=7.8 Hz, 3H), 1.22 (t, J=7.2 Hz, 3H), 1.61 (q, J=7.5 Hz, 2H), 2.56 (q, J=7.2 Hz, 2H), 3.74 (t, J=7.2 Hz, 2H), 4.13 (q, J=7.2 Hz, 2H), 6.14 (s, 2H).

### Step 2: 5-Amino-3-ethyl-1-propyl-1H-pyrazole-4-carboxylic acid

The titled intermediate was prepared by the ester hydrolysis of Step 1 intermediate (19.0 g, 84.44 mmol) using aqueous solution of potassium hydroxide (9.5 g, 168.8 mmol in 140 mL water) in EtOH (190 mL) as per the procedure described in Step 3 of Intermediate 1. The solvent was then evaporated under reduced pressure obtained crude was carried forward to the next step.

### Step 3: 2-(2,4-Difluorophenyl)-2-oxoethyl 5-amino-3-ethyl-1-propyl-1H-pyrazole-4-carboxylate

The titled compound was prepared by the reaction of Step 2 intermediate (2.0 g, 10.15 mmol) with 2-bromo-1-(2,4-difluorophenyl)ethanone (2.6 g, 11.17 mmol) using potassium fluoride (884 mg, 15.23 mmol) in dry DMF (20 mL) as per the procedure described in Step 4 of Intermediate 1 to yield 1.4 g of the product as a solid. ¹H NMR (500 MHz, DMSO-d₆): δ 0.84 (t, J=7.5 Hz, 3H), 1.11 (t, J=7.5 Hz, 3H), 1.66 (q, J=7.5 Hz, 2H), 2.61 (t, J=7.5 Hz, 2H), 3.79 (t, J=7.0 Hz, 2H), 5.34 (s, 2H), 6.29 (s, 2H), 7.26-7.32 (m, 2H), 7.47-7.53 (m, 2H), 7.96-8.03 (m, 2H).

### Intermediate 178

2-(2,6-Difluorophenyl)-2-oxoethyl 5-amino-3-ethyl-1-propyl-1H-pyrazole-4-carboxylate

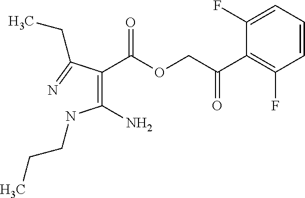 [SMILES: CCCN/C(N)=C(\C(=N)CC)C(=O)OCC(=O)c1c(F)cccc1F]

The titled compound was prepared by the reaction of Step 2 of intermediate 177 (2.0 g, 10.15 mmol) with 2-bromo-1-(2,6-difluorophenyl)ethanone (2.6 g, 11.17 mmol) using potassium fluoride (884 mg, 15.23 mmol) in dry DMF (20 mL) as per the procedure described in Step 4 of Intermediate 1 to yield 1.9 g of the product as a solid. ¹H NMR (500 MHz, DMSO-d₆): δ 0.83 (t, J=7.5 Hz, 3H), 1.07 (t, J=7.5 Hz, 3H), 1.63 (q, J=7.0 Hz, 2H), 2.55 (q, J=8.0 Hz, 2H), 3.77 (t, J=7.0 Hz, 2H), 5.19 (s, 2H), 6.28 (s, 2H), 7.26 (t, J=8.5 Hz, 2H), 7.63-7.71 (m, 1H).

### Intermediate 179

2-(2,6-Difluorophenyl)-2-oxoethyl 5-amino-3-ethyl-1-(2-(4-ethylpiperazin-1-yl)ethyl)-1H-pyrazole-4-carboxylate

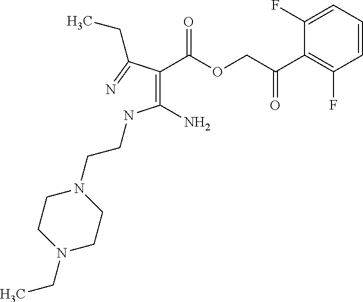 [SMILES: CCC(=N)/C(C(=O)OCC(=O)c1c(F)cccc1F)=C(/N)NCCN1CCN(CC)CC1]

### Step 1: Ethyl 5-amino-3-ethyl-1-(4-ethylpiperazin-1-yl)-1H-pyrazole-4-carboxylate

The titled compound was prepared by the reaction of (E)-ethyl 2-cyano-3-ethoxypent-2-enoate (6.7 g, 33.92 mmol) with 1-ethyl-4-(2-hydrazinylethyl)piperazine (7.0 g, 40.70 mmol) using N,N-Diisopropylethylamine (11.70 mL, 67.83 mmol) in ethanol (67 mL) as per the procedure described in Step 1 of Intermediate 75 to yield 1.05 g of the product as oil. ¹H NMR (400 MHz, DMSO-d₆): δ 0.91-1.21 (m, 6H), 1.25 (t, J=6.9 Hz, 3H), 2.48-2.64 (m, 10H), 2.58 (t, J=6.9 Hz, 2H), 3.90-4.15 (m, 4H), 4.17 (t, J=6.8 Hz, 2H), 6.27 (s, 2H).

### Step 2: 5-Amino-3-ethyl-1-(4-ethylpiperazin-1-yl)-1H-pyrazole-4-carboxylic acid

The titled intermediate was prepared by the ester hydrolysis of Step 1 intermediate (1.00 g, 3.09 mmol) using aqueous solution of potassium hydroxide (346 mg, 6.18 mmol in 2 mL water) in EtOH (20 mL) as per the procedure described in Step 3 of Intermediate 1. The solvent was then evaporated under reduced pressure to obtain a mixture which was carried forward to the next reaction.

### Step 3: 2-(2,6-Difluorophenyl)-2-oxoethyl 5-amino-3-ethyl-1-(4-ethylpiperazin-1-yl)-1H-pyrazole-4-carboxylate

The titled compound was prepared by the reaction of Step 2 intermediate (300 g, 3.06 mmol) with 2-bromo-1-(2,6-difluorophenyl)ethanone (863 g, 3.97 mmol) using potassium fluoride (266 mg, 4.59 mmol) in dry DMF (9 mL) as per the procedure described in Step 4 of Intermediate 1 to yield 420 mg of the product as a solid. ¹H NMR (400 MHz, DMSO-d₆): δ 0.99 (t, J=7.2 Hz, 3H), 1.07 (q, J=7.6 Hz, 3H), 2.41-2.6 (m, 10H), 3.95 (t, J=6.8 Hz, 2H), 5.20 (s, 2H), 6.41 (s, 2H), 7.27(t, J=8.8 Hz, 2H), 7.68 (q, J=8.4 Hz 1H).

### Intermediate 180

2-(2-Chlorophenyl)-2-oxoethyl 5-amino-3-ethyl-1-(2-(4-ethylpiperazin-1-yl)ethyl)-1H-pyrazole-4-carboxylate

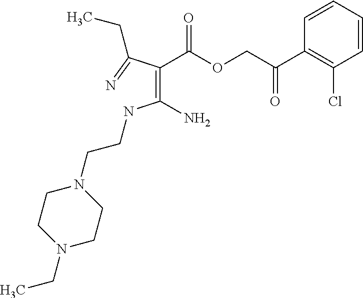 [SMILES: CCC(=N)/C(C(=O)OCC(=O)c1ccccc1Cl)=C(/N)NCCN1CCN(CC)CC1]

The titled compound was prepared by the reaction of Step 2 Intermediate 179 (1.2 g, 4.08 mmol) with 2-bromo-1-(2-chlorophenyl)ethanone (1.1 g, 4.89 mmol) using potassium fluoride (355 mg, 6.12 mmol) in dry DMF (12 mL) as per the procedure described in Step 4 of Intermediate 1 to yield 615 mg of the product as liquid. ¹H NMR (400 MHz, DMSO-d₆): δ 0.89-1.34 (m, 6H), 2.53-2.84 (m, 12H), 2.79 (t, J=4.8 Hz, 3H), 4.06 (t, J=4.4 Hz, 2H), 5.36 (s, 2H), 6.40 (s, 2H), 7.39 (t, J=7.6 Hz, 1H), 7.45-7.48 (m, 2H), 7.65-7.69 (m, 1H).

### Intermediate 181

2-(2-Fluoro-3-(trifluoromethyl)phenyl)-2-oxoethyl 5-amino-3-ethyl-1-(3-morpholinopropyl)-1H-pyrazole-4-carboxylate

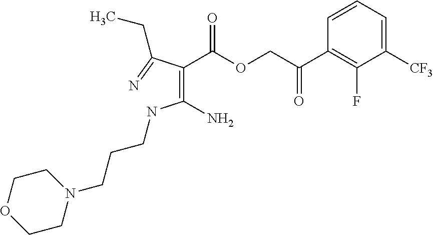 [SMILES: CCC(=N)/C(C(=O)OCC(=O)c1cccc(C(F)(F)F)c1F)=C(/N)NCCCN1CCOCC1]

### Step 1: Ethyl 5-amino-3-ethyl-1-(3-morpholinopropyl)-1H-pyrazole-4-carboxylate

The titled compound was prepared by the reaction of (E)-ethyl 2-cyano-3-ethoxypent-2-enoate (9.0 g, 45.6 mmol) with 4-(3-hydrazinylpropyl)morpholine (8.8 g, 54.75 mmol) using N,N-Diisopropylethylamine (15.6 mL, 9.12 mmol) in dry ethanol (90 mL) as per the procedure described in Step 1 of Intermediate 75 to yield 5.15 g of the product as sticky oil. ¹H NMR (400 MHz, DMSO-d₆)): δ 1.23 (t, J=7.2 Hz, 3H), 1.36 (t, J=7.2 Hz, 3H), 2.00-2.08 (m, 2H), 2.33 (t, J=6.0 Hz, 2H), 2.41-2.58 (m, 4H), 2.75 (q, J=7.6 Hz, 2H), 3.78 (t, J=4.8 Hz, 4H), 3.97 (t, J=6.0 Hz, 2H), 4.29 (q, J=7.2 Hz, 2H), 6.16 (br s, 2H). APCI (m/z) 312 (M+H)⁺.

### Step 2: 5-Amino-3-ethyl-1-(3-morpholinopropyl)-1H-pyrazole-4-carboxylic acid

The titled intermediate was prepared by the ester hydrolysis of Step 1 intermediate (5.1 g, 16.4 mmol) using aqueous solution of potassium hydroxide (1.84 g, 32.89 mmol 9.0 mL water) in EtOH (50 mL) as per the procedure described in Step 3 of Intermediate 1. The solvent was then evaporated and co-distilled with isopropanol (4×25 ml) to yield 2.15 g of the product as sticky oil which was directly used for the next step.

### Step 3: 2-(2-Fluoro-3-(trifluoromethyl)phenyl)-2-oxoethyl 5-amino-3-ethyl-1-(3-morpholinopropyl)-1H-pyrazole-4-carboxylate

The titled compound was prepared by the reaction of Step 2 intermediate (1.3 g, 4.0 mmol) with 2-bromo-1-(2-fluoro-3-(trifluoromethyl)phenyl)ethanone (1.45 g, 5.0 mmol) using potassium fluoride (401 mg, 6.9 mmol) in dry DMF (20 mL) as per the procedure described in Step 4 of Intermediate 1 to yield 730 mg of the product as a solid. ¹H NMR (400 MHz, DMSO-d₆): δ 1.11 (t, J=7.2 Hz, 3H), 1.82 (t, J=6.8 Hz, 2H), 2.25 (t, J=6.8 Hz, 2H), 2.28-2.36 (m, 4H), 2.62 (q, J=7.6 Hz, 2H), 3.58 (t, J=4.8 Hz, 4H), 3.86 (t, J=6.8 Hz, 2H), 5.39 (s, 2H), 6.34 (s, 2H), 7.60 (t, J=7.6 Hz, 1H), 8.11 (t, J=6.4 Hz, 1H), 8.19 (t, J=6.8 Hz, 1H); APCI (m/z) 487 (M+H)⁺.

### Intermediate 182

2-(2,6-Difluorophenyl)-2-oxoethyl 5-amino-3-ethyl-1-(3-morpholinopropyl)-1H-pyrazole-4-carboxylate

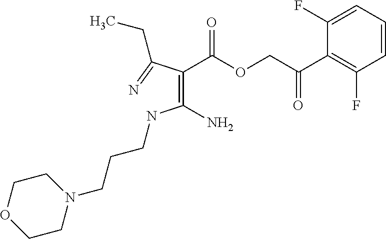 [SMILES: CCC(=N)/C(C(=O)OCC(=O)c1c(F)cccc1F)=C(/N)NCCCN1CCOCC1]

The titled compound was prepared by the reaction of Step 2 of intermediate 181 (900 mg, 3.19 mmol) with 2-bromo-1-(2,6-difluorophenyl)ethanone (825 g, 3.50 mmol) using potassium fluoride (280 mg, 4.78 mmol) in dry DMF (20 mL) as per the procedure described in Step 4 of Intermediate 1 to yield 350 mg of the product. ¹H NMR (400 MHz, CDCl₃): δ 1.19-1.43 (m, 3H), 2.03-2.10 (m, 2H), 2.32-2.46 (m, 2H), 2.50-2.68 (m, 4H), 2.73 (t, J=7.2 Hz, 2H), 3.75-3.90 (m, 4H), 4.01 (t, J=6.0 Hz, 2H), 5.22 (s, 1H), 7.01 (t, J=8.4 Hz, 2H), 7.44-7.49 (m, 1H).

### Intermediate 183

2-(2,4-Difluorophenyl)-2-oxoethyl 5-amino-3-ethyl-1-(3-morpholinopropyl)-1H-pyrazole-4-carboxylate

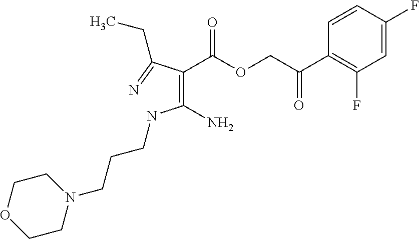 [SMILES: CCC(=N)/C(C(=O)OCC(=O)c1ccc(F)cc1F)=C(/N)NCCCN1CCOCC1]

The titled compound was prepared by the reaction of Step 2 of intermediate 181 (800 mg, 3.19 mmol) with 2-bromo-1-(2,4-difluorophenyl)ethanone (735 g, 3.11 mmol) using potassium fluoride (250 mg, 4.25 mmol) in dry DMF (8 mL) as per the procedure described in Step 4 of Intermediate 1 to yield 395 mg of the product. ¹H NMR (400 MHz, DMSO-d₆): δ 1.12 (t, J=7.2 Hz, 3H), 1.83 (t, J=6.8 Hz, 2H), 2.25 (t, J=7.2 Hz, 2H), 2.29-2.37 (m, 4H), 2.63 (q, J=7.6 Hz, 2H), 3.58 (t, J=4.8 Hz, 4H), 3.86 (t, J=6.8 Hz, 2H), 5.33 (s, 2H), 6.33 (s, 2H), 7.30 (t, J=8.4 Hz, 1H), 7.51 (t, J=6.8 Hz, 1H), 8.0 (q, J=6.8 Hz, 1H).

### Intermediate 184

2-(2-Chlorophenyl)-2-oxoethyl 5-amino-3-methyl-1-(2-(piperazin-1-yl)ethyl)-1H-pyrazole-4-carboxylate

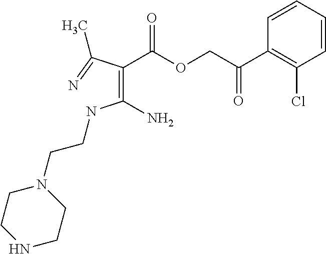 [SMILES: CC(=N)/C(C(=O)OCC(=O)c1ccccc1Cl)=C(/N)NCCN1CCNCC1]

### Step 1: tert-Butyl 4-(2-hydrazinylethyl)piperazine-1-carboxylate

To a stirred solution of tert-butyl 4-(2-chloroethyl)piperazine-1-carboxylate (9.7 g, 38.9 mmol) in ethanol (25 ml) was added hydrazine hydrate (19.5 ml, 38.9 mmol) and resulting recation mixturte was heated to 60° C. for 3 h. The solvent was evaporated under reduced pressure, diluted with water, extracted with diethyl ether (75 ml×4) and the organic extract was dried over Na₂SO₄ and concentrated under reduced pressure to give 9.6 g of the desired product as colorless oil. ¹H NMR (400 MHz, DMSO-d₆): δ 1.45 (s, 9H), 2.38-2.45 (m, 4H), 2.52 (t, J=6.0 Hz, 2H), 2.88 (t, J=6.0 Hz, 2H), 3.04 (br, s, 3H), 3.39-3.69 (m, 4H).

### Step 2: tert-Butyl 4-(2-(5-amino-4-(ethoxycarbonyl)-3-methyl-1H-pyrazol-1-yl)ethyl)piperazine-1-carboxylate

The titled compound was prepared by the reaction of (E)-ethyl 2-cyano-3-ethoxypent-2-enoate (8.5 g, 43.7 mmol) with tert-Butyl 4-(2-hydrazinylethyl)piperazine-1-carboxylate (step 1 intermediate, 11.7 g, 48.07 mmol) using N,N-Diisopropylethylamine (15 mL, 87.4 mmol) in dry ethanol (65 mL) as per the procedure described in Step 1 of Intermediate 75 to yield 13 g of the product as sticky oil. ¹H NMR (400 MHz, DMSO-d₆)): δ 1.36 (t, J=7.2 Hz, 3H), 1.47 (s, 9H), 2.32 (s, 3H), 2.50-2.57 (m, 4H), 2.78 (t, J=4.4 Hz, 2H), 3.47 (t, J=4.8 Hz, 4H), 4.07 (t, J=4.8 Hz, 2H), 4.28 (q, J=7.2 Hz, 2H), 6.17 (s, 2H).

### Step 3: 5-Amino-1-(2-(4-(tert-butoxycarbonyl)piperazin-1-yl)ethyl)-3-methyl-1H-pyrazole-4-carboxylic acid

The titled intermediate was prepared by the ester hydrolysis of Step 2 intermediate (9.5 g, 24.9 mmol) using aqueous solution of potassium hydroxide (2.8 g, 49.8 mmol, 20 mL water) in EtOH (95 mL) as per the procedure described in Step 3 of Intermediate 1. The solvent was then evaporated and co-distilled with isopropanol (4×25 ml) to yield 8.7 g of the product as sticky oil which was directly used for the next step.

### Step 4: tert-Butyl 4-(2-(5-amino-4-((2-(2-chlorophenyl)-2-oxoethoxy)carbonyl)-3-methyl-1H-pyrazol-1-yl)ethyl)piperazine-1-carboxylate

The titled compound was prepared by the reaction of Step 3 Intermediate (8.7 g, 24.6 mmol) with 2-bromo-1-(2-chlorophenyl)ethanone (6.4 g, 27.07 mmol) using potassium fluoride (2.2 g, 36.92 mmol) in dry DMF (85 mL) as per the procedure described in Step 4 of Intermediate 1 to yield 8.9 g of the product as liquid. ¹H NMR (400 MHz, DMSO-d₆): δ 1.48 (s, 9H), 2.30 (s, 3H), 2.59-2.64 (m, 4H), 2.87-2.98 (m, 2H), 3.50-3.57 (m, 4H), 4.12-4.17 (m, 2H), 5.35 (s, 2H), 6.31 (s, 2H), 7.27-7.42 (m, 17.45-7.48 (m, 2H), 7.64-7.69 (m, 1H).

### Step 5: 2-(2-Chlorophenyl)-2-oxoethyl 5-amino-3-methyl-1-(2-(piperazin-1-yl)ethyl)-1H-pyrazole-4-carboxylate

To s stirred solution of Step 4 Intermediate (11.3 g, 22.37 mmol) in dry ethyl acetate (50 ml) was added dry saturated hydrochloric acid in ethyl acetate (200 ml) at 0° C. and resulting mixture was stirred at room temperature for overnight. The solvent was evaporated under reduced pressure and basified with saturated solution of NaHCO₃ and extracted with ethyl acetate (150 ml×3) and combinmed organic layer was dried over Na2SO4 and concentrated to give 8.9 of the product as oil. ¹H NMR (400 MHz, DMSO-d₆): δ 2.13 (s, 3H), 2.58-2.67 (m, 6H), 2.98 (t, J=4.8 Hz, 4H), 3.94 (t, J=6.4 Hz, 2H), 5.31 (s, 2H), 6.37 (s, 2H), 7.47-7.53 (m, 1H), 7.57-7.61 (m, 2H), 7.76-7.80 (m, 1H), 8.12 (br, s, 1H). ESI (m/z) 406 (M+H)⁺.

### Intermediate 185

2-(2-Fluoro-3-(trifluoromethyl)phenyl)-2-oxoethyl 5-amino-3-methyl-1-(2-(4-methylpiperazin-1-yl)ethyl)-1H-pyrazole-4-carboxylate

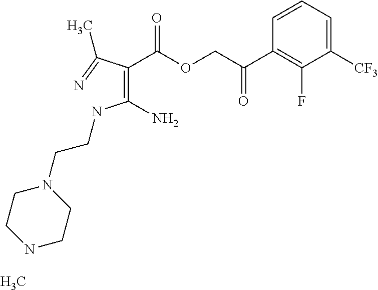

The titled compound was prepared by the reaction of Step 2 intermediate 162 (1.0 g, 3.74 mmol) with 2-bromo-1-(2-fluoro-3-(trifluoromethyl)phenyl)ethanone (1.17 g, 4.11 mmol) using potassium fluoride (326 mg, 5.61 mmol) in dry DMF (10 mL) as per the procedure described in Step 4 of Intermediate 1 for 3 h to yield 330 mg of the product as sticky oil. ¹H NMR (500 MHz, DMSO-d₆): δ 1.98 (s, 3H), 2.17 (s, 3H), 2.21-2.52 (m, 8H), 2.59 (t, J=8.0 Hz, 2H), 3.94 (t, J=6.5 Hz, 2H), 5.38 (s, 2H), 6.39 (s, 2H), 7.59 (t, J=8.0 Hz, 2H), 8.10 (t, J=8.0 Hz, 1H), 8.19 (t, J=8.0 Hz).

### Intermediate 186

2-(2-Fluoro-3-(trifluoromethyl)phenyl)-2-oxoethyl 5-amino-3-ethyl-1-(2-(4-methylpiperazin-1-yl)ethyl)-1H-pyrazole-4-carboxylate

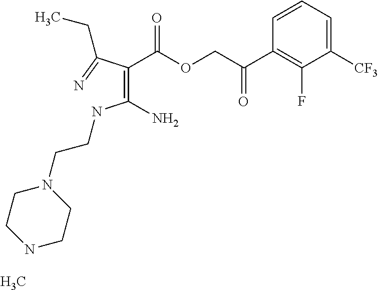

The titled compound was prepared by the reaction of Step 2 intermediate 172 (1.8 g, 6.39 mmol) with 2-bromo-1-(2-fluoro-3-(trifluoromethyl)phenyl)ethanone (2.00 g, 7.03 mmol) using potassium fluoride (620 mg, 10.67 mmol) in dry DMF (20.0mL) as per the procedure described in Step 4 of Intermediate 1 for 4 h to yield 800 mg of the product. ¹H NMR (300 MHz, DMSO-d₆): δ 1.10 (t, J=7.6 Hz, 3H), 2.16 (s, 3H), 2.20-2.65 (m, 12H), 3.95 (t, J=6.4 Hz, 2H), 5.38 (s, 2H), 6.42 (br s, 2H), 7.59 (t, J=7.6 Hz, 1H), 8.10 (t, J=6.8 Hz, 1H), 8.20 (t, J=6.4 Hz, 1H). ESI (m/z) 486 (M+H)⁺.

### Intermediate 187

2-(2,6-Difluorophenyl)-2-oxoethyl 5-amino-3-ethyl-1-is opentyl-1H-pyrazole-4-carboxylate

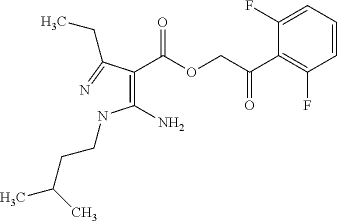 [SMILES: CCC(=N)/C(C(=O)OCC(=O)c1c(F)cccc1F)=C(/N)NCCC(C)C]

### Step 1: ethyl 5-amino-3-ethyl-1-isopentyl-1H-pyrazole-4-carboxylate

The titled compound was prepared by the reaction of (E)-ethyl 2-cyano-3-ethoxypent-2-enoate (6.0 g, 32.69 mmol) with isopentylhydrazine (2.9 g, 39.23 mmol) using N,N-Diisopropylethylamine (11.0 mL, 65.38 mmol) in IPA (60 mL) as per the procedure described in Step 1 of Intermediate 75 to yield 1.61 g of the product as sticky oil. ¹H NMR (400 MHz, DMSO-d₆)): δ 0.89 (d, J=6.0 Hz, 6H), 1.09 (t, J=7.6 Hz, 3H), 1.23 (t, J=7.2 Hz, 3H), 1.50-1.54 (m, 2H), 2.50 (br s, 2H), 2.54 (q, J=7.2 Hz, 2H), 3.82 (t, J=7.2 Hz, 1H), 4.15 (q, J=6.8 Hz, 2H), 6.16 (s, 2H).

### Step 2: 5-Amino-3-ethyl-1-isopentyl-1H-pyrazole-4-carboxylic acid

The titled intermediate was prepared by the ester hydrolysis of Step 1 intermediate (1.6 g, 6.42 mmol) using aqueous solution of potassium hydroxide (719 mg, 12.85 mmol 1.0 mL water) in EtOH (16 mL) as per the procedure described in Step 3 of Intermediate 1 to yield 1.10 g of the product as off-white solid. ¹H NMR (400 MHz, DMSO-d₆)): δ 0.89 (t, J=6.4 Hz, 6H), 1.09 (t, J=7.6 Hz, 3H), 1.52 (br s, 3H), 2.58 (q, J=7.60 Hz, 2H), 3.80 (t, J=6.8 Hz, 2H), 6.13 (s, 2H), 11.69 (br s, 1H).

### Step 3: 2-(2,6-Difluorophenyl)-2-oxoethyl 5-amino-3-ethyl-1-isopentyl-1H-pyrazole-4-carboxylate

The titled compound was prepared by the reaction of Step 2 intermediate (1.1 g, 4.97 mmol) with 2-bromo-1-(2,4-difluorophenyl)ethanone (1.28 g, 5.47 mmol) using potassium fluoride (432 mg, 7.46 mmol) in dry DMF (10 mL) as per the procedure described in Step 4 of Intermediate 1 to yield 1.25 g of the product as a brown coloured oil. ¹H NMR (400 MHz, DMSO-d₆): δ 0.88 (d, J=6.8 Hz, 6H), 1.05 (t, J=4.8 Hz, 3H), 1.51-1.54 (m, 3H), 2.49 (q, J=7.6 Hz, 2H), 3.83 (d, J=7.2 Hz, 2H), 5.20 (s, 2H), 6.28 (s, 2H), 7.26 (t, J=8.4 Hz, 2H), 7.65-7.69 (m, 1H).

### Intermediate 188

2-(2-Chlorophenyl)-2-oxoethyl 5-amino-3-ethyl-1-is opentyl-1H-pyrazole-4-carboxylate

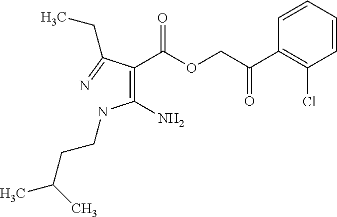 [SMILES: CCC(=N)/C(C(=O)OCC(=O)c1ccccc1Cl)=C(/N)NCCC(C)C]

The titled compound was prepared by the reaction of Step 2 intermediate 187 5-amino-3-ethyl-1-isopentyl-1H-pyrazole-4-carboxylic acid (1.0 g, 4.52 mmol) with 2-bromo-1-(2-chlorophenyl)ethanone (1.16 g, 4.97 mmol) using potassium fluoride (394 mg, 6.78 mmol) in dry DMF (10.0 mL) as per the procedure described in Step 4 of Intermediate 1 for 4 h to yield 950 mg of the product. ¹H NMR (400 MHz, DMSO-d₆): δ 0.90 (d, J=6.0 Hz, 6H), 1.05-1.09 (m, 3H), 1.49-1.51 (m, 2H), 2.49-2.51 (m, 3H), 3.83 (t, J=7.2 Hz, 2H), 5.31 (s, 2H), 6.28 (s, 2H), 7.50-7.56 (m, 1H), 7.58-7.60 (m, 2H), 7.79 (t, J=1.2 Hz, 1H).

### Intermediate 189

2-(2,6-Difluorophenyl)-2-oxoethyl 5-amino-3-(methoxymethyl)-1-propyl-1H-pyrazole-4-carboxylate

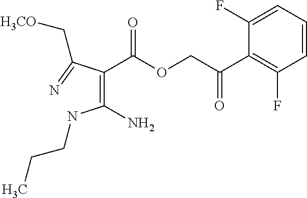 [SMILES: CCCN/C(N)=C(\C(=N)COC)C(=O)OCC(=O)c1c(F)cccc1F]

### Step 1: 5-Amino-3-(methoxymethyl)-1-propyl-1H-pyrazole-4-carbonitrile

The titled compound was prepared by the reaction of 2-(1,2-dimethoxyethylidene)malononitrile (8.2 g, 53.96 mmol) with propylhydrazine (4.0 g, 53.96 mmol) using N,N-Diisopropylethylamine (18.6 mL, 107.9 mmol) in dry ethanol (80 mL) as per the procedure described in Step 1 of Intermediate 75 to yield 7.30 g of the product as sticky oil. ¹H NMR (400 MHz, DMSO-d₆)): δ 0.81 (t, J=7.6 Hz, 3H), 3.21 (d, J=3.2 Hz, 3H), 3.33 (s, 2H), 3.80 (t, J=7.20 Hz, 2H), 4.21 (s, 2H), 6.75 (s, 2H); ESI (m/z) 195 (M+H)⁺.

### Step 2: 5-Amino-3-(methoxymethyl)-1-propyl-1H-pyrazole-4-carboxylic acid

A suspension of Step 1 intermediate (7.3 g, 37.62 mmol) and sodium hydroxide (9.0 g, 225.7 mmol) in water (90 mL) was heated at 90° C. for 72 h. The mixture was cooled to RT and acidified with 1N citric acid till pH 2-3. The aqueous layer was extracted with ethyl acetate (75 mL×2) and the organic layer was dried over anhydrous sodium sulfate. The solution was concentrated under reduced pressure to obtain 3.8 g of the titled product as a solid. ¹H NMR (400 MHz, DMSO-d₆): δ 0.82 (t, J=4.40 Hz, 3H), 1.63-1.68 (m, 2H), 3.18 (d, J=5.20 Hz, 3H), 3.79 (t, J=7.20 Hz, 2H), 4.35 (s, 2H), 6.21 (s, 2H), 11.91 (br s, 1H).

### Step 3: 2-(2,6-Difluorophenyl)-2-oxoethyl 5-amino-3-(methoxymethyl)-1-propyl-1H-pyrazole-4-carboxylate

The titled compound was prepared by the reaction of Step 2 intermediate (1.8 g, 8.45 mmol) with 2-bromo-1-(2,4-difluorophenyl)ethanone (2.2 g, 9.29 mmol) using potassium fluoride (735 mg, 12.67 mmol) in dry DMF (20 mL) as per the procedure described in Step 4 of Intermediate 1 to yield 2.2 g of the product as a solid. ¹H NMR (400 MHz, DMSO-d₆): δ 0.84 (t, J=7.2 Hz, 3H), 1.14-1.69 (m, 2H), 3.17 (d, J=5.2 Hz, 3H), 3.84 (t, J=6.8 Hz, 2H), 4.32 (s, 2H), 5.21 (s, 2H), 6.37 (s, 2H), 7.24 (t, J=8.4 Hz, 2H), 7.66-7.740 (m, 1H).

### Intermediate 190

2-(2,6-Difluorophenyl)-2-oxoethyl 5-amino-1-cyclopropyl-3-(methoxymethyl)-1H-pyrazole-. 4-carboxylate

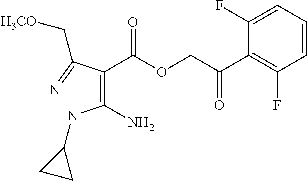 [SMILES: COCC(=N)/C(C(=O)OCC(=O)c1c(F)cccc1F)=C(/N)NC1CC1]

### Step 1: 5-Amino-1-cyclopropyl-3-(methoxymethyl)-1H-pyrazole-4-carbonitrile

The titled compound was prepared by the reaction of 2-(1,2-dimethoxyethylidene)malononitrile (8.0 g, 52.59 mmol) with cyclopropylhydrazine (6.85 g, 63.06 mmol) using N,N-Diisopropylethylamine (18.0 mL, 105 mmol) in dry ethanol (83 mL) as per the procedure described in Step 1 of Intermediate 75 to yield 4.9 g of the product as sticky oil. ¹H NMR (400 MHz, CDCl₃): δ 1.13-1.15 (m, 2), 1.68-1.70 (m, 2H), 3.10-3.12 (m, 1H), 3.44 (s, 3H), 4.39 (s, 2H), 4.60-4.70 (brs, 2H).

### Step 2: 5-Amino-1-cyclopropyl-3-(methoxymethyl)-1H-pyrazole-4-carboxylic acid

The titled compound was prepared by the reaction of Step 1 intermediate (4.9 g, 25.49 mmol) with sodium hydroxide (6.19 g, 152.9 mmol) in water (50 mL) as per the procedure described in Step 2 of Intermediate 96 to yield 2.1 g of the desired product as a solid. ¹H NMR (400 MHz, DMSO-d₆): δ 0.95-0.96 (m, 4H), 3.22-3.31 (m, 1H), 3.32 (s, 3H), 4.32 (s, 2H), 6.21 (s, 2H), 11.94 (br s, 1H).

### Step 3: 2-(2,6-Difluorophenyl)-2-oxoethyl 5-amino-1-cyclopropyl-3-(methoxymethyl)-1H-pyrazole-4-carboxylate

The titled compound was prepared by the reaction of Step 2 intermediate (2.1 g, 9.94 mmol) with 2-bromo-1-(2,6-difluorophenyl)ethanone (2.57 g, 10.33 mmol) using potassium fluoride (866 mg, 14.91 mmol) in dry DMF (20 mL) as per the procedure described in Step 4 of Intermediate 1 to yield 1.12 g of the product as a solid. ¹H NMR (400 MHz, DMSO-d₆): δ 0.93-0.98 (m, 4H), 3.28-3.30 (m, 1H), 3.33 (s, 3H), 4.30 (s, 2H), 5.21 (s, 2H), 6.39 (s, 2H), 7.27 (td, J₁=2.0 Hz, J₂=8.8 Hz, 2H), 7.66-7.698 (m, 1H).

### Intermediate 191

2-(2,6-Difluorophenyl)-2-oxoethyl 5-amino-1-(2-(2,2-dimethyl-3-oxomorpholino)ethyl)-3-ethyl-1H-pyrazole-4-carboxylate

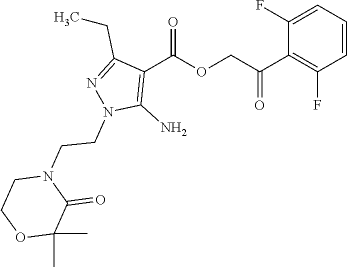 [SMILES: CCc1nn(CCN2CCOC(C)(C)C2=O)c(N)c1C(=O)OCC(=O)c1c(F)cccc1F]

### Step 1: Ethyl 5-amino-1-(2-(2,2-dimethyl-3-oxomorpholino)ethyl)-3-ethyl-1H-pyrazole-4-carboxylate

The titled compound was prepared by the reaction of 2-(1,2-dimethoxyethylidene)malononitrile (7.7 g, 41.9 mmol) with 4-(2-hydrazinylethyl)-2,2-dimethylmorpholin-3-one (9.5 g, 50.35 mmol) using N,N-Diisopropylethylamine (14.4 mL, 83.9 mmol) in dry ethanol (77 mL) as per the procedure described in Step 1 of Intermediate 75 to yield 2.74 g of the product as sticky oil. ¹H NMR (400 MHz, DMSO-d₆)): δ 1.10 (t, J=7.6 Hz, 3H), 1.23-1.26 (m, 9H), 2.59 (q, J=7.2 Hz, 2H), 3.07 (t, J=5.2 Hz, 2H), 3.51 (t, J=6.0 Hz, 2H), 3.67 (t, J=4.8 Hz, 2H), 4.02 (t, J=5.6 Hz, 2H), 4.15 (q, J=7.2 Hz, 2H), 6.22 (s, 2H); APCI (m/z) 312 (M+H)⁺.

### Step 2: 5-Amino-1-(2-(2,2-dimethyl-3-oxomorpholino)ethyl)-3-ethyl-1H-pyrazole-4-carboxylic acid

The titled intermediate was prepared by the ester hydrolysis of Step 1 intermediate (3.0 g, 8.90 mmol) using aqueous solution of potassium hydroxide (1.99 g, 35.6 mmol 6.0 mL water) in EtOH (30 mL) as per the procedure described in Step 3 of Intermediate 1 to yield 2.4 g of the crude product carry forwarded as it is for next step.

### Step 3: 2-(2,6-Difluorophenyl)-2-oxoethyl 5-amino-1-(2-(2,2-dimethyl-3-oxomorpholino)ethyl)-3-ethyl-1H-pyrazole-4-carboxylate

The titled compound was prepared by the reaction of Step 2 intermediate (2.37 g, 7.66 mmol) with 2-bromo-1-(2,6-difluorophenyl)ethanone (667 mg, 11.50 mmol) using potassium fluoride (1.98 g, 8.43 mmol) in dry DMF (25 mL) as per the procedure described in Step 4 of Intermediate 1 to yield 1.02 g of the product as a solid. ¹H NMR (400 MHz, DMSO-d₆): δ 1.08 (t, J=7.6 Hz, 3H), 1.23 (s, 6H), 2.54 (q, J=7.6 Hz, 2H), 3.08 (t, J=4.8 Hz, 2H), 3.52 (t, J=5.6 Hz, 2H), 3.67 (t, J=5.2 Hz, 2H), 4.03 (t, J=5.6 Hz, 2H), 5.20 (s, 2H), 6.35 (s, 2H), 7.23-7.29 (m, 2H), 7.64-7.71 (m, 1H).

### Intermediate 192

2-(2,6-Difluorophenyl)-2-oxoethyl 5-amino-1-methyl-3-(1-(methylsulfonyl)piperidin-4-yl)-1H-pyrazole-4-carboxylate

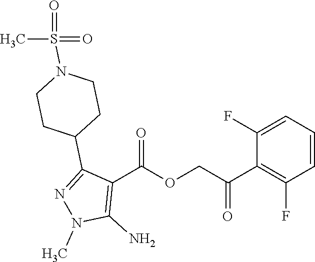 [SMILES: Cn1nc(C2CCN(S(C)(=O)=O)CC2)c(C(=O)OCC(=O)c2c(F)cccc2F)c1N]

### Step 1: 5-Amino-1-methyl-3-(1-(methylsulfonyl)piperidin-4-yl)-1H-pyrazole-4-carbonitrile

The titled compound was prepared by the reaction of 2-(methoxy(l-(methylsulfonyl)piperidin-4-yl)methylene)malononitrile (4.7 g, 0.017 mol) with methylhydrazine sulphate (2.52 g, 0.017 mol) using N,N-Diisopropylethylamine (6.0 mL, 0.034 mol) in dry ethanol (50 mL) as per the procedure described in Step 1 of Intermediate 75 to yield 1.83 g of the product as sticky oil. ¹H NMR (400 MHz, DMSO-d₆)): δ 1.62-1.72 (m, 2H), 1.89-1.93 (m, 2H), 2.60-2.67 (m, 1H), 2.78-2.87 (m, 2H), 2.92 (s, 3H), 3.47 (s, 3H), 3.52-3.61 (m, 2H), 6.52 (s, 2H).

### Step 2: 5-Amino-1-methyl-3-(1-(methylsulfonyl)piperidin-4-yl)-1H-pyrazole-4-carboxylic acid

The titled compound was prepared by the reaction of Step 1 intermediate (1.9 g, 6.70 mmol) with sodium hydroxide (1.60 g, 40.23 mmol) in water (20 mL) as per the procedure described in Step 2 of Intermediate 96 to yield 1.37 g of the desired product as a solid. ¹H NMR (300 MHz, DMSO-d₆): δ 1.63-1.67 (m, 2H), 1.91-1.99 (m, 2H), 2.48 (s, 2H), 2.86 (s, 3H), 3.17-3.84 (m, 6H), 6.08-6.15 (br s, 2H), 11.78-12.00 (br s, 1H).

### Step 3: 2-(2,6-Difluorophenyl)-2-oxoethyl 5-amino-1-methyl-3-(1-(methylsulfonyl)piperidin-4-yl)-1H-pyrazole-4-carboxylate

The titled compound was prepared by the reaction of Step 2 intermediate (1.3 g, 4.29 mmol) with 2-bromo-1-(2,4-difluorophenyl)ethanone (1.11 g, 4.72 mmol) using potassium fluoride (374 mg, 6.44 mmol) in dry DMF (10 mL) as per the procedure described in Step 4 of Intermediate 1 to yield 1.06 g of the product as a solid. ¹H NMR (400 MHz, DMSO-d₆): δ 1.57-1.61 (m, 2H), 1.89-1.99 (m, 2H), 2.71-2.77 (m, 2H), 2.87 (s, 3H), 2.98-3.34 (m, 1H), 3.50 (s, 3H), 3.58-3.61 (m, 2H), 5.20 (s, 2H), 6.32 (s, 2H), 7.28 (t, J=0.8 Hz, 2H), 7.67-7.71 (m, 1H).

### Intermediate 193

2-(2,6-difluorophenyl)-2-oxoethyl 5-amino-1-cyclopropyl-3-(1-(methylsulfonyl)piperidin-4-yl)-1H-pyrazole-4-carboxylate

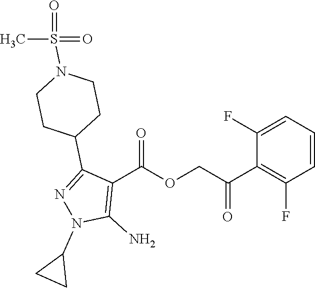 [SMILES: CS(=O)(=O)N1CCC(c2nn(C3CC3)c(N)c2C(=O)OCC(=O)c2c(F)cccc2F)CC1]

### Step 1: 5-Amino-1-cyclopropyl-3-(1-(methylsulfonyl)piperidin-4-yl)-1H-pyrazole-4-carbonitrile

The titled compound was prepared by the reaction of 2-(methoxy(1-(methylsulfonyl)piperidin-4-yl)methylene)malononitrile (4.0 g, 0.014 mol) with cyclopropyl hydrazine hydrochloride (2.14 g, 0.014 mol) using N,N-Diisopropylethylamine (5.1 mL, 0.029 mol) in dry ethanol (40 mL) as per the procedure described in Step 1 of Intermediate 75 to yield 1.41 g of the product as sticky oil. ¹H NMR (400 MHz, DMSO-d₆)): δ 0.93-0.96 (m, 4H), 1.62-1.87 (m, 2H), 1.87-1.93 (m, 2H), 2.59-2.65 (m, 1H), 2.77-2.86 (m, 3H), 3.13-3.20 (m, 1H), 3.33-3.46 (m, 2H), 3.61 (d, J=10.0 Hz, 2H), 6.60 (s, 2H).

### Step 2: 5-Amino-1-cyclopropyl-3-(1-(methylsulfonyl)piperidin-4-yl)-1H-pyrazole-4-carboxylic acid

The titled compound was prepared by the reaction of Step 1 intermediate (1.4 g, 4.520 mmol) with sodium hydroxide (1.08 g, 27.14 mmol) in water (15 mL) as per the procedure described in Step 2 of Intermediate 96 to yield 430 mg of the desired product as a solid. ¹H NMR (300 MHz, DMSO-d₆): δ 0.93-1.17 (m, 4H), 1.57-1.66 (m, 2H), 1.89-1.99 (m, 2H), 2.72-3.35 (m, 7H), 3.57 (d, J=11.6 Hz, 2H), 6.10-6.16 (br s, 2H), 11.76-11.92 (br s, 1H).

### Step 3: 2-(2,6-Difluorophenyl)-2-oxoethyl 5-amino-1-cyclopropyl-3-(1-(methylsulfonyl)piperidin-4-yl)-1H-pyrazole-4-carboxylate

The titled compound was prepared by the reaction of Step 2 intermediate (420 mg, 1.27 mmol) with 2-bromo-1-(2,4-difluorophenyl)ethanone (330 mg, 1.40 mmol) using potassium fluoride (112 mg, 1.91 mmol) in dry DMF (10 mL) as per the procedure described in Step 4 of Intermediate 1 to yield 420 mg of the product as a solid. ¹H NMR (400 MHz, DMSO-d₆): δ 0.96-0.98 (m, 4H), 1.54-1.87 (m, 2H), 1.89-1.91 (m, 2H), 2.70-2.99 (m, 5H), 3.16-3.20 (m, 2H), 3.45 (d, J=11.6 Hz, 2H), 5.20 (s, 2H), 6.35 (s, 2H), 7.25 (t, J=8.4 Hz, 2H), 7.65-7.72 (m, 1H).

### Intermediate 194

2-(2-(2,4-Difluorophenyl)thiazol-5-yl)-2-oxoethyl 5-amino-3-ethyl-1-methyl-1H-pyrazole-4-carboxylate

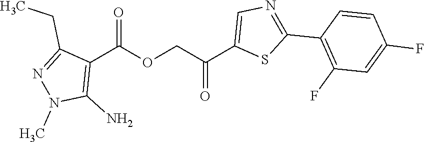 [SMILES: CCc1nn(C)c(N)c1C(=O)OCC(=O)c1cnc(-c2ccc(F)cc2F)s1]

The titled compound was prepared by the reaction of Step 2 intermediate 55 (880 mg, 5.71 mmol) with 2-bromo-1-(2-(2,4-difluorophenyl)thiazol-5-yl)ethanone (2.0 g, 6.28 mmol) using potassium fluoride (497 mg, 8.56 mmol) in dry DMF (10 mL) as per the procedure described in Step 4 of Intermediate 1 to yield 1.81 g of the product as a solid. ¹H NMR (400 MHz, DMSO-d₆): δ 1.12 (t, J=7.6 Hz, 3H), 2.61 (q, J=7.6 Hz, 2H), 3.49 (s, 3H), 5.48 (s, 2H), 6.30 (s, 2H), 7.34 (t, J=2.4 Hz, 1H), 7.58-7.63 (m, 1H), 8.37 (q, J=2.4 Hz, 1H), 8.92 (s, 1H); ESI (m/z) 407 (M+H)⁺.

### Intermediate 195

2-(2,6-Difluorophenyl)-2-oxoethyl 5-amino-3-(methoxymethyl)-1-methyl-1H-pyrazole-4-carboxylate

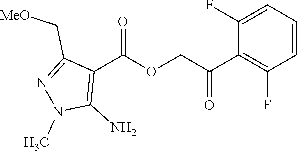 [SMILES: COCc1nn(C)c(N)c1C(=O)OCC(=O)c1c(F)cccc1F]

The titled compound was prepared by the reaction of Step 2 intermediate 107 (490 mg, 2.64 mmol) with 2-bromo-1-(2,6-difluorophenyl)ethanone (746 mg, 3.17 mmol) using potassium fluoride (230 mg, 3.96 mmol) in dry DMF (10 mL) as per the procedure described in Step 4 of Intermediate 1 to yield 310 mg of the product as a solid. ¹H NMR (400 MHz, DMSO-d₆): δ 3.21 (s, 3H), 3.52 (s, 3H), 4.32 (s, 2H), 5.21 (s, 2H), 6.34 (s, 2H), 7.26 (t, J=8.8 Hz, 2H), 7.65-7.70 (m, 1H).

### Intermediate 196

2-(2,6-Difluorophenyl)-2-oxoethyl 5-amino-1-methyl-3-(tetrahydro-2H-pyran-4-yl)-1H-pyrazole-4-carboxylate

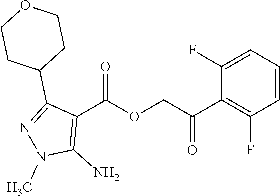 [SMILES: Cn1nc(C2CCOCC2)c(C(=O)OCC(=O)c2c(F)cccc2F)c1N]

### Step 1: 5-Amino-1-methyl-3-(tetrahydro-2H-pyran-4-yl)-1H-pyrazole-4-carbonitrile

The titled compound was prepared by the reaction of 2-(methoxy(tetrahydro-2H-pyran-4-yl)methylene)malononitrile (4.6 g, 23.95 mmol) with methyl hydrazine sulfate (3.7 g, 23.95 mmol) using N,N-Diisopropylethylamine (8.3 mL, 47.9 mmol) in dry ethanol (50 mL) as per the procedure described in Step 1 of Intermediate 75 to yield 2.81 g of the product as yellow solid. ¹H NMR (400 MHz, DMSO-d₆): δ 1.42-1.73 (m, 4H), 2.71-2.77 (m, 1H), 3.21-3.46 (m, 5H), 3.86-3.90 (m, 2H), 6.47 (s, 2H).

### Step 2: 5-Amino-1-methyl-3-(tetrahydro-2H-pyran-4-yl)-1H-pyrazole-4-carboxylic acid

The titled compound was prepared by the reaction of Step 1 intermediate (2.8 g, 13.59 mmol) with sodium hydroxide (3.3 g, 81.54 mmol) in water (33 mL) as per the procedure described in Step 2 of Intermediate 96 to yield 1.30 g of the desired product as white solid. ¹H NMR (300 MHz, DMSO-d₆): δ 1.57-1.70 (m, 4H), 2.55-2.67 (m, 1H), 3.11-3.32 (m, 2H), 3.52 (s, 3H), 3.88-3.89 (m, 2H), 6.13 (s, 2H), 11.91-11.94 (br s, 1H).

### Step 3: 2-(2,6-Difluorophenyl)-2-oxoethyl 5-amino-1-methyl-3-(tetrahydro-2H-pyran-4-yl)-1H-pyrazole-4-carboxylate

The titled compound was prepared by the reaction of Step 2 intermediate (660 mg, 2.93 mmol) with 2-bromo-1-(2,6-difluorophenyl)ethanone (758 mg, 3.22 mmol) using potassium fluoride (255 mg, 4.39 mmol) in dry DMF (7.0 mL) as per the procedure described in Step 4 of Intermediate 1 to yield 503 mg of the product as a sticky solid. ¹H NMR (400 MHz, DMSO-d₆): δ 1.57-1.70 (m, 4H), 3.08-3.15 (m, 1H), 3.17 (d, J=5.2 Hz, 2H), 3.35 (s, 3H), 3.85-3.88 (m, 2H), 5.19 (s, 2H), 6.30 (s, 2H), 7.24-7.28 (m, 2H), 7.65-7.69 (m, 1H).

### Intermediate 197

2-(2,6-Difluorophenyl)-2-oxoethyl 5-amino-1-cyclopropyl-3-(tetrahydro-2H-pyran-4-yl)-1H-pyrazole-4-carboxylate

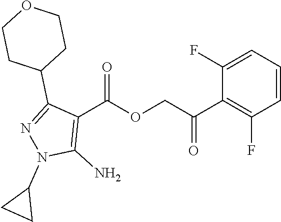 [SMILES: Nc1c(C(=O)OCC(=O)c2c(F)cccc2F)c(C2CCOCC2)nn1C1CC1]

### Step 1: 5-Amino-1-cyclopropyl-3-(tetrahydro-2H-pyran-4-yl)-1H-pyrazole-4-carbonitrile

The titled compound was prepared by the reaction of 2-(methoxy(tetrahydro-2H-pyran-4-yl)methylene)malononitrile (4.0 g, 20.83 mmol) with cyclopropyl hydrazine hydrochloride (3.0 g, 20.83 mmol) using N,N-Diisopropylethylamine (7.2 mL, 41.66 mmol) in dry ethanol (40 mL) as per the procedure described in Step 1 of Intermediate 75 to yield 2.31 g of the product as yellow solid. ¹H NMR (400 MHz, DMSO-d₆): δ 0.90-1.0 (m, 4H), 1.49-1.70 (m, 4H), 2.70-2.73 (m, 1H), 3.13-3.40 (m, 3H), 3.85-3.90 (m, 2H), 6.56 (s, 2H); ESI (m/z) 233 (M+H)⁺.

### Step 2: 5-Amino-1-cyclopropyl-3-(tetrahydro-2H-pyran-4-yl)-1H-pyrazole-4-carboxylic acid

The titled compound was prepared by the reaction of Step 1 intermediate (2.0 g, 8.62 mmol) with sodium hydroxide (2.06 g, 51.72 mmol) in water (20 mL) as per the procedure described in Step 2 of Intermediate 96 to yield 1.07 g of the desired product as a pale yellow solid. ¹H NMR (300 MHz, DMSO-d₆): δ 0.90-1.0 (m, 4H), 1.61-1.91 (m, 4H), 3.12-3.17 (m, 1H), 3.28-3.44 (m, 2H), 3.82-3.88 (m, 2H), 12.16 (br s, 2H); ESI (m/z) 251 (M+H)⁺.

### Step 3: 2-(2,6-Difluorophenyl)-2-oxoethyl 5-amino-1-cyclopropyl-3-(tetrahydro-2H-pyran-4-yl)-1H-pyrazole-4-carboxylate

The titled compound was prepared by the reaction of Step 2 intermediate (500 mg, 1.99 mmol) with 2-bromo-1-(2,6-difluorophenyl)ethanone (561 mg, 2.39 mmol) using potassium fluoride (173 mg, 2.98 mmol) in dry DMF (5.0 mL) as per the procedure described in Step 4 of Intermediate 1 to yield 551 mg of the product as brown oil. ¹H NMR (400 MHz, DMSO-d₆): δ 0.91-0.96 (m, 4H), 1.56-1.65 (m, 4H), 3.06-3.19 (m, 2H), 3.28-3.33 (m, 2H), 3.84-3.87 (m, 2H), 5.19 (s, 2H), 6.32 (s, 2H), 7.26 (dt, J₁=2.0 Hz, J₂=10.4 Hz, 2H), 7.64-7.71 (m, 1H); ESI (m/z) 406 (M+H)⁺.

### Intermediate 198

2-(2-Chlorophenyl)-2-oxoethyl 5-amino-1-methyl-3-(tetrahydro-2H-pyran-4-yl)-1H-pyrazole-4-carboxylate

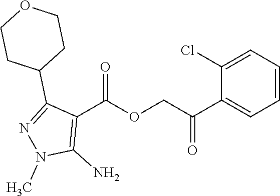 [SMILES: Cn1nc(C2CCOCC2)c(C(=O)OCC(=O)c2ccccc2Cl)c1N]

The titled compound was prepared by the reaction of Step 2 intermediate 196 5-amino-3-ethyl-1-isopentyl-1H-pyrazole-4-carboxylic acid (810 mg, 3.60 mmol) with 2-bromo-1-(2-chlorophenyl)ethanone (930 mg, 3.96 mmol) using potassium fluoride (315 mg, 5.4 mmol) in dry DMF (10.0 mL) as per the procedure described in Step 4 of Intermediate 1 for 4 h to yield 850 mg of the product as sticky solid. ¹H NMR (400 MHz, DMSO-d₆): δ 1.58-1.71 (m, 4H), 3.08-3.14 (m, 1H), 3.30-3.49 (m, 2H), 3.51 (s, 3H), 3.85-3.88 (m, 2H), 5.31 (s, 2H), 6.30 (s, 2H), 7.47-7.51 (m, 1H), 7.58-7.59 (m, 2H), 7.68-7.79 (m, 1H).

### Intermediate 199

2-(2-Chlorophenyl)-2-oxoethyl 5-amino-1-methyl-3-(1-(methylsulfonyl)piperidin-4-yl)-1H-pyrazole-4-carboxylate

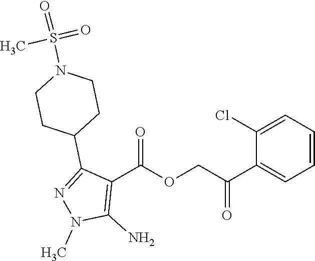 [SMILES: Cn1nc(C2CCN(S(C)(=O)=O)CC2)c(C(=O)OCC(=O)c2ccccc2Cl)c1N]

The titled compound was prepared by the reaction of Step 2 intermediate 192 (950 mg, 3.14 mmol) with 2-bromo-1-(2-chlorophenyl)ethanone (806 g, 3.46 mmol) using potassium fluoride (274 mg, 4.71 mmol) in dry DMF (10.0 mL) as per the procedure described in Step 4 of Intermediate 1 to yield 630 mg of the product as yellow solid. ¹H NMR (400 MHz, DMSO-d₆): δ 1.57-1.63 (m, 2H), 1.89-1.99 (m, 2H), 2.50-2.51 (m, 2H), 2.94 (s, 3H), 2.95-3.01 (m, 1H), 3.51 (m, 3H), 3.57-3.60 (m, 2H), 5.32 (s, 2H), 6.31 (s, 2H), 7.49-7.52 (m, 1H), 7.59-7.60 (m, 2H), 7.28 (d, J=7.6 Hz, 1H).

### Intermediate 200

2-(2-Chlorophenyl)-2-oxoethyl 5-amino-3-(methoxymethyl)-1-methyl-1H-pyrazole-4-carboxylate

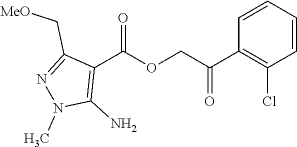 [SMILES: COCc1nn(C)c(N)c1C(=O)OCC(=O)c1ccccc1Cl]

The titled compound was prepared by the reaction of Step 2 intermediate 107 (1.0 g, 5.40 mmol) with 2-bromo-1-(2-chlorophenyl)ethanone (1.4 g, 5.94 mmol) using potassium fluoride (470 mg, 8.1 mmol) in dry DMF (10 mL) as per the procedure described in Step 4 of Intermediate 1 to yield 870 mg of the product as a solid. ¹H NMR (400 MHz, DMSO-d₆): δ 3.19 (s, 3H), 3.55 (s, 3H), 4.34 (s, 2H), 5.32 (s, 2H), 6.33 (s, 2H), 7.47-7.51 (m, 1H), 7.57-7.59 (m, 2H), 7.79 (d, J=7.2 Hz, 1H).

### Intermediate 201

2-(2,6-Difluorophenyl)-2-oxoethyl 5-amino-1-(2-((2R,6S)-2,6-dimethylmorpholino)ethyl)-3-ethyl-1H-pyrazole-4-carboxylate

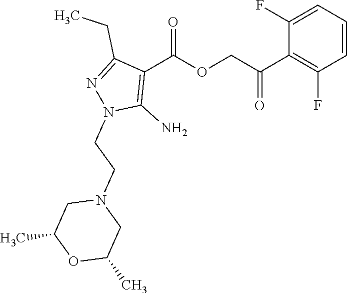 [SMILES: CCc1nn(CCN2C[C@@H](C)O[C@@H](C)C2)c(N)c1C(=O)OCC(=O)c1c(F)cccc1F]

### Step 1: Ethyl 5-amino-1-(2-((2R,6S)-2,6-dimethylmorpholino)ethyl)-3-ethyl-1H-pyrazole-4-carboxylate

The titled compound was prepared by the reaction of (E)-ethyl 2-cyano-3-ethoxypent-2-enoate (5.3 g, 0.026 mmol) with (2R,6S)-4-(hydrazinylmethyl)-2,6-dimethylmorpholine (5.57 g, 0.032 mmol) using N,N-Diisopropylethylamine (9.26 mL, 0.053 mol) in dry ethanol (55 mL) as per the procedure described in Step 1 of Intermediate 75 to yield 4.7 g of the product as sticky oil. ¹H NMR (400 MHz, DMSO-d₆): δ 1.03 (d, J=6.4 Hz, 2H), 1.09 (t, J=7.6 Hz, 6H), 1.23 (t, J=6.8 Hz, 4H), 1.67 (t, J=10.8 Hz, 2H), 2.50-2.62 (m, 4H), 2.79 (d, J=10.8 Hz, 2H), 3.51-3.54 (m, 2H), 3.94 (t, J=6.8 Hz, 2H), 4.16 (q, J=7.2 Hz, 2H), 6.27 (s, 2H).

### Step 2: 5-Amino-1-(2-((2R,6S)-2,6-dimethylmorpholino)ethyl)-3-ethyl-1H-pyrazole-4-carboxylic acid

The titled compound was prepared by the reaction of Step 1 intermediate (4.6 g, 0.014 mmol) with potassium hydroxide (1.67 g, 0.029 mmol) in water (17 mL) and ethanol (34 mL) as per the procedure described in Step 3 of Intermediate 1 to yield 2.83 g of the desired product as a solid. ¹H NMR (400 MHz, DMSO-d₆)): δ 1.04 (d, J=6.4 Hz, 3H), 1.09 (t, J=7.6 Hz, 6H), 1.68 (t, J=10.8 Hz, 2H), 1.99 (s, 2H), 2.49-2.61 (m, 2H), 2.79 (d, J=10.4 Hz, 2H), 3.50-3.55 (m, 2H), 3.93 (t, J=6.8 Hz, 2H), 6.23 (s, 2H), 11.76 (br s, 1H).

### Step 3: 2-(2,6-Difluorophenyl)-2-oxoethyl 5-amino-1-(2-((2R,6S)-2,6-dimethylmorpholino)ethyl)-3-ethyl-1H-pyrazole-4-carboxylate

The titled compound was prepared by the reaction of Step 2 intermediate (1.50 g, 5.06 mmol) with 2-bromo-1-(2,6-difluorophenyl)ethanone (1.42 g, 6.08 mmol) using potassium fluoride (441 mg, 7.60 mmol) in dry DMF (15 mL) as per the procedure described in Step 4 of Intermediate 1 to yield 1.03 g of the product as a solid. ¹H NMR (400 MHz, DMSO-d₆)): δ 1.03-1.09 (m, 9H), 1.68 (t, J=10.4 Hz, 2H), 2.49-2.58 (m, 4H), 2.80 (d, J=10.8 Hz, 2H), 3.51-3.54 (m, 2H), 3.96 (t, J=6.4 Hz, 2H), 5.20 (s, 2H), 5.76 (s, 2H), 7.24-7.29 (m, 2H), 7.65-7.69 (m, 1H).

### Intermediate 202

2-(2-Chlorophenyl)-2-oxoethyl 5-amino-1-(2-((2R, 6S)-2,6-dimethylmorpholino)ethyl)-3-ethyl-1H-pyrazole-4-carboxylate

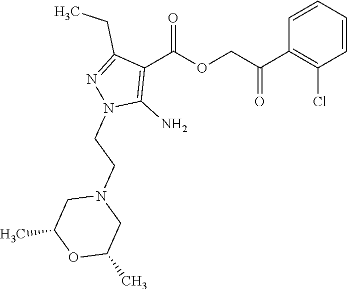 [SMILES: CCc1nn(CCN2C[C@@H](C)O[C@@H](C)C2)c(N)c1C(=O)OCC(=O)c1ccccc1Cl]

The titled compound was prepared by the reaction of Step 2 intermediate 201 (1.30 g, 4.39 mol) with 2-bromo-1-(2-chlorophenyl)ethanone (1.23 g, 5.27 mol) using potassium fluoride (382 mg, 6.58 mmol) in dry DMF (13 mL) as per the procedure described in Step 4 of Intermediate 1 to yield 1.1 g of the product as a solid. ¹H NMR (400 MHz, DMSO-d₆): δ 1.03-1.09 (m, 9H), 1.67 (t, J=10.8 Hz, 2H), 2.50-2.59 (m, 4H), 2.80 (t, J=10.8 Hz, 2H), 3.51-3.54 (m, 2H), 3.96 (t, J=10.8 Hz, 2H), 5.76 (s, 2H), 6.39 (s, 2H), 7.47-7.51 (m, 1H), 7.58-7.59 (m, 2H), 7.78 (d, J=7.2 Hz, 1H).

### Intermediate 203

2-(2,6-Difluorophenyl)-2-oxoethyl 5-amino-3-ethyl-1-(2-hydroxyethyl)-1H-pyrazole-4-carboxylate

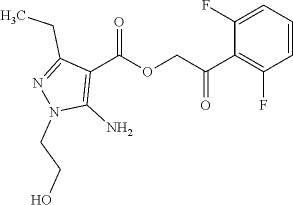 [SMILES: CCc1nn(CCO)c(N)c1C(=O)OCC(=O)c1c(F)cccc1F]

### Step 1: Ethyl 5-amino-3-ethyl-1-(2-hydroxyethyl)-1H-pyrazole-4-carboxylate

The titled compound was prepared by the reaction of (E)-ethyl 2-cyano-3-ethoxypent-2-enoate (5.0 g, 0.025 mol) with 2-hydroxy ethylhydrazine (2.31 g, 0.030 mol) using N,N-Diisopropylethylamine (8.6 mL, 0.050 mol) in dry ethanol (50 mL) as per the procedure described in Step 1 of Intermediate 75 to yield 4.46 g of the product as sticky oil. ¹H NMR (400 MHz, DMSO-d₆)): δ 1.11 (dt, J₁=1.2 Hz, J₂=5.4 Hz, 3H), 1.25 (dt, J₁=1.2 Hz, J₂=6.8 Hz, 3H), 2.60 (q, J=6.4 Hz, 2H), 3.88 (t, J=6.0 Hz, 2H), 4.14 (t, J=5.6 Hz, 2H), 4.18 (q, J=6.0 Hz, 2H), 4.92 (br s, 1H), 6.07 (s, 2H).

### Step 2: 5-Amino-3-ethyl-1-(2-hydroxyethyl)-1H-pyrazole-4-carboxylic acid

The titled compound was prepared by the reaction of Step 1 intermediate (4.4 g, 0.019 mol) with potassium hydroxide (2.1 g, 0.029 mol) in water (15 mL) and ethanol (30 mL) as per the procedure described in Step 3 of Intermediate 1 to yield 2.73 g of the desired product as a solid. ¹H NMR (300 MHz, DMSO-d₆): δ 1.10 (t, J=7.2 Hz, 3H), 2.59 (q, J=5.2 Hz, 2H), 3.64 (q, J=5.2 Hz, 2H), 3.87 (t, J=6.0 Hz, 2H), 4.92 (t, J=5.2 Hz, 1H), 6.04 (s, 2H), 11.80 (s, 1H).

### Step 3: 2-(2,6-Difluorophenyl)-2-oxoethyl 5-amino-3-ethyl-1-(2-hydroxyethyl)-1H-pyrazole-4-carboxylate

The titled compound was prepared by the reaction of Step 2 intermediate (2.70 g, 0.013 mol) with 2-bromo-1-(2,6-difluorophenyl)ethanone (3.82 g, 0.016 mol) using potassium fluoride (1.18 g, 0.020 mol) in dry DMF (27 mL) as per the procedure described in Step 4 of Intermediate 1 to yield 1.78 g of the product as a solid. ¹H NMR (400 MHz, DMSO-d₆): δ 1.08 (t, J=7.2 Hz, 3H), 2.56 (q, J=7.2 Hz, 2H), 3.66 (q, J=5.6 Hz, 2H),3.89 (t, J=4.8 Hz, 2H), 4.93 (t, J=5.2 Hz, 1H), 5.20 (s, 2H), 6.20 (s, 2H), 7.26 (t, J=8.4 Hz, 2H), 7.65-7.69 (m, 1H)

### Intermediate 204

2-(2,6-Difluorophenyl)-2-oxoethyl 5-amino-1-(4-fluorophenyl)-3-(tetrahydro-2H-pyran-4-yl)-1H-pyrazole-4-carboxylate

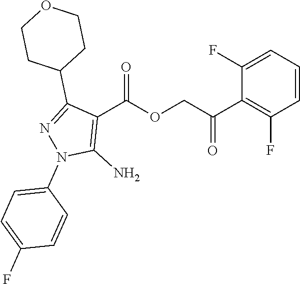 [SMILES: Nc1c(C(=O)OCC(=O)c2c(F)cccc2F)c(C2CCOCC2)nn1-c1ccc(F)cc1]

### Step 1: 5-Amino-1-(4-fluorophenyl)-3-(tetrahydro-2H-pyran-4-yl)-1H-pyrazole-4-carbonitrile

The titled compound was prepared by the reaction of 2-(methoxy(tetrahydro-2H-pyran-4-yl)methylene)malononitrile (3.2 g, 16.6 mmol) with 4-fluorophenyl hydrazine hydrochloride (2.7 g, 16.6 mmol) using N,N-Diisopropylethylamine (5.7 mL, 33.2 mmol) in dry ethanol (32 mL) as per the procedure described in Step 1 of Intermediate 75 to yield 3.58 g of the product as sticky oil. ¹H NMR (400 MHz, DMSO-d₆)): δ 1.70-1.81 (m, 2H), 2.81-2.89 (m, 1H), 3.28-3.62 (m, 4H), 3.90-3.92 (m, 2H), 6.65 (m, 2H), 7.32-7.37 (m, 2H), 7.49-7.53 (m, 2H).

### Step 2: 5-Amino-1-(4-fluorophenyl)-3-(tetrahydro-2H-pyran-4-yl)-1H-pyrazole-4-carboxylic acid

The titled compound was prepared by the reaction of Step 1 intermediate (3.5 g, 12.2 mnmol) with sodium hydroxide (2.9 g, 73.3 mmol) in water (29 mL) as per the procedure described in Step 2 of Intermediate 96 to yield 2.73 g of the desired product as a solid. ¹H NMR (300 MHz, DMSO-d₆): δ 1.67-1.73 (m, 3H), 3.24-3.41 (m, 4H), 3.89 (m, 2H), 6.30 (br s, 2H), 7.35 (t, J=8.8 Hz, 2H), 7.54-7.57 (m, 2H), 12.17 (br s, 1H).

### Step 3: 2-(2,6-Difluorophenyl)-2-oxoethyl 5-amino-1-(4-fluorophenyl)-3-(tetrahydro-2H-pyran-4-yl)-1H-pyrazole-4-carboxylate

The titled compound was prepared by the reaction of Step 2 intermediate (1.0 g, 3.27 mmol) with 2-bromo-1-(2,6-difluorophenyl)ethanone (924 mg, 3.93 mmol) using potassium fluoride (285 mg, 4.90 mmol) in dry DMF (10 mL) as per the procedure described in Step 4 of Intermediate 1 to yield 905 mg of the product as a solid. ¹H NMR (400 MHz, DMSO-d₆): δ 1.64-1.68 (m, 2H), 1.70-1.79 (m, 2H), 3.16-3.20 (m, 1H), 3.34-3.39 (m, 2H), 3.88-3.91 (m, 2H), 5.27 (s, 2H), 6.44 (s, 2H), 7.26-7.35 (m, 2H), 7.35-7.39 (m, 2H), 7.54-7.58 (m, 2H), 7.67-7.72 (m, 1H).

### Intermediate 205

2-(2,6-Difluorophenyl)-2-oxoethyl 5-amino-1-(2-(4,4-difluoropiperidin-1-yl)ethyl)-3-ethyl-1H-pyrazole-4-carboxylate

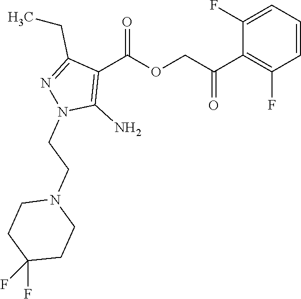 [SMILES: CCc1nn(CCN2CCC(F)(F)CC2)c(N)c1C(=O)OCC(=O)c1c(F)cccc1F]

### Step 1: Ethyl 5-amino-1-(2-(4,4-difluoropiperidin-1-yl)ethyl)-3-ethyl-1H-pyrazole-4-carboxylate

The titled compound was prepared by the reaction of (E)-ethyl 2-cyano-3-methoxypent-2-enoate (2.5 g, 12.67 mmol) with 4,4-difluoro-1-(2-hydrazinylethyl)piperidine (2.72 g, 15.2 mmol) using N,N-Diisopropylethylamine (4.4 mL, 25.3 mmol) in dry ethanol (25 mL) as per the procedure described in Step 1 of Intermediate 75 to yield 1.55 g of the product as sticky oil. ¹H NMR (400 MHz, DMSO-d₆)): δ 1.21-1.41 (m, 6H), 2.03-2.12 (m, 4H), 2.72-2.84 (m, 6H), 2.93-2.96 (m, 2H), 4.16-4.20 (m, 2H), 4.30 (q, J=7.2 Hz, 2H), 6.15 (s, 2H); ESI (m/z) 331 (M+H)⁺.

### Step 2: 5-Amino-1-(2-(4,4-difluoropiperidin-1-yl)ethyl)-3-ethyl-1H-pyrazole-4-carboxylic acid

The titled compound was prepared by the reaction of Step 1 intermediate (1.5 g, 4.5 mnmol) with potassium hydroxide (510 mg, 6.08 mmol) in water (50 mL) and ethanol (15 mL) as per the procedure described in Step 3 of Intermediate 1 to yield 190 mg of the desired product as a solid. ¹H NMR (300 MHz, DMSO-d₆): δ 1.09 (t, J=7.6 Hz, 3H), 1.88-1.99 (m, 4H), 2.50-2.61 (m, 6H), 2.67 (t, J=6.4 Hz, 2H), 3.93 (t, J=6.8 Hz, 2H), 6.22 (s, 2H), 11.72 (s, 1H); ESI (m/z) 303 (M+H)⁺.

### Step 3: 2-(2,6-Difluorophenyl)-2-oxoethyl 5-amino-1-(2-(4,4-difluoropiperidin-1-yl)ethyl)-3-ethyl-1H-pyrazole-4-carboxylate

The titled compound was prepared by the reaction of Step 2 intermediate (870 mg, 2.87 mmol) with 2-bromo-1-(2,6-difluorophenyl)ethanone (820 mg, 3.45 mmol) using potassium fluoride (250 mg, 4.31 mmol) in dry DMF (9 mL) as per the procedure described in Step 4 of Intermediate 1 to yield 340 mg of the product as a solid. ¹H NMR (400 MHz, DMSO-d₆): δ 1.07 (t, J=7.6 Hz, 3H), 1.88-1.98 (m, 4H), 2.50-2.68 (m, 4H), 2.73 (s, 2H), 2.89 (s, 2H), 3.96 (t, J=6.8 Hz, 2H), 5.20 (s, 2H), 6.38 (s, 2H), 7.26 (t, J=8.4 Hz, 2H), 7.64-7.71 (m, 1H); ESI (m/z) 457 (M+H)⁺.

### Intermediate 206

2-(2,6-Difluorophenyl)-2-oxoethyl 5-amino-1-(3-((2R,6S)-2,6-dimethylmorpholino)propyl)-3-ethyl-1H-pyrazole-4-carboxylate

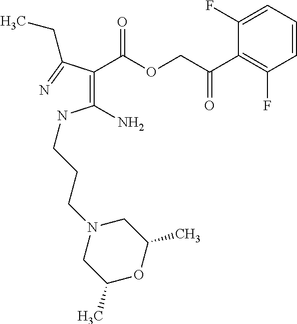 [SMILES: CCC(=N)/C(C(=O)OCC(=O)c1c(F)cccc1F)=C(/N)NCCCN1C[C@@H](C)O[C@@H](C)C1]

### Step 1: Ethyl 5-amino-1-(3-((2R,6S)-2,6-dimethylmorpholino)propyl)-3-ethyl-1H-pyrazole-4-carboxylate

The titled compound was prepared by the reaction of (E)-ethyl 2-cyano-3-methoxypent-2-enoate (7.0 g, 0.035 mol) with (2R,6S)-4-(3-hydrazinylpropyl)-2,6-dimethylmorpholine (7.96 g, 0.042 mol) using N,N-Diisopropylethylamine (12.3 mL, 0.070 mol) in dry ethanol (70 mL) as per the procedure described in Step 1 of Intermediate 75 to yield 3.0 g of the product as sticky oil. ¹H NMR (400 MHz, DMSO-d₆): δ 1.02-1.06 (m, 8H), 1.10 (t, J=7.6 Hz, 2H), 1.25 (t, J=7.2 Hz, 2H), 1.53 (t, J=10.4 Hz, 2H), 1.78-1.81 (m, 2H), 2.20 (t, J=6.8 Hz, 2H), 2.59 (q, J=7.6 Hz, 2H), 2.69 (d, J=10.8 Hz, 2H), 3.51-3.56 (m, 2H), 3.82 (t, J=6.8 Hz, 2H), 4.16 (q, J=6.8 Hz, 2H), 6.19 (s, 2H).

### Step 2: 5-Amino-1-(3-((2R,6S)-2,6-dimethylmorpholino)propyl)-3-ethyl-1H-pyrazole-4-carboxylic acid

The titled compound was prepared by the reaction of Step 1 intermediate (3.0 g, 9.25 mnmol) with potassium hydroxide (1.03 g, 18.51 mmol) in water (12 mL) and ethanol (23 mL) as per the procedure described in Step 3 of Intermediate 1 to yield 1.32 g of the desired product as a solid.

### Step 3: 2-(2,6-Difluorophenyl)-2-oxoethyl 5-amino-1-(2-((2S,6R)-2,6-dimethylmorpholino)ethyl)-3-ethyl-1H-pyrazole-4-carboxylate

The titled compound was prepared by the reaction of Step 2 intermediate (1.30 g, 4.19 mol) with 2-bromo-1-(2,6-difluorophenyl)ethanone (1.18 g, 5.03 mol) using potassium fluoride (365 mg, 6.29 mmol) in dry DMF (10 mL) as per the procedure described in Step 4 of Intermediate 1 to yield 314 mg of the product as a solid. ¹H NMR (400 MHz, DMSO-d₆): δ 0.84-1.10 (m, 9H), 1.54 (t, J=10.0 Hz, 2H), 1.79-1.91 (m, 2H), 2.15-2.21 (m, 2H), 2.70 (d, J=10.4 Hz, 2H), 3.53-3.56 (m, 2H), 3.82-3.85 (m, 2H), 5.20 (s, 2H), 5.76 (s, 2H), 6.32 (s, 2H), 7.26 (t, J=8.4 Hz, 2H), 7.66-7.69 (m, 1H).

### Intermediate 207

2-(2,6-Difluorophenyl)-2-oxoethyl 5-amino-3-ethyl-1-((tetrahydro-2H-pyran-4-yl)methyl)-1H-pyrazole-4-carboxylate

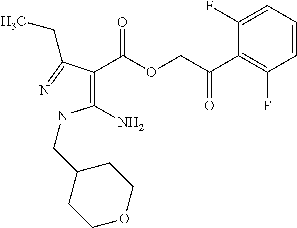 [SMILES: CCC(=N)/C(C(=O)OCC(=O)c1c(F)cccc1F)=C(/N)NCC1CCOCC1]

### Step 1: Ethyl 5-amino-3-ethyl-1-((tetrahydro-2H-pyran-4-yl)methyl)-1H-pyrazole-4-carboxylate

The titled compound was prepared by the reaction of (E)-ethyl 2-cyano-3-methoxypent-2-enoate (3.2 g, 16.22 mmol) with ((tetrahydro-2H-pyran-4-yl)methyl)hydrazine (2.53 g, 19.47 mmol) using N,N-Diisopropylethylamine (5.6 mL, 32.44 mmol) in dry ethanol (732mL) as per the procedure described in Step 1 of Intermediate 75 to yield 3.02 g of the product as sticky oil. ¹H NMR (400 MHz, DMSO-d₆)): δ 1.18-1.57 (m, 10H), 1.99-2.02 (m, 1H), 2.59 (q, J=7.6 Hz, 2H), 3.17-3.29 (m, 4H), 3.73 (d, J=7.2 Hz, 2H), 3.80-3.83 (m, 2H), 6.20 (s, 2H).

### Step 2: 5-Amino-3-ethyl-1-((tetrahydro-2H-pyran-4-yl)methyl)-1H-pyrazole-4-carboxylic acid

The titled compound was prepared by the reaction of Step 1 intermediate (3.50 g, 12.45 mnmol) with potassium hydroxide (1.7 g, 24.91 mmol) in water (20 mL) and ethanol (35 mL) as per the procedure described in Step 3 of Intermediate 1 to yield 3.05 g of the desired product as a solid. ¹H NMR (400 MHz, DMSO-d₆): δ 1.21 (t, J=7.6 Hz, 3H), 1.24-1.42 (m, 4H), 1.98-1.99 (m, 1H), 2.59 (q, J=7.6 Hz, 2H), 3.16-3.28 (m, 2H), 3.70-3.82 (m, 4H), 6.17 (s, 2H), 11.86 (br s, 1H).

### Step 3: 2-(2,6-Difluorophenyl)-2-oxoethyl 5-amino-3-ethyl-1-((tetrahydro-2H-pyran-4-yl)methyl)-1H-pyrazole-4-carboxylate

The titled compound was prepared by the reaction of Step 2 intermediate (1.0 g, 3.95 mol) with 2-bromo-1-(2,6-difluorophenyl)ethanone (1.10 g, 4.74 mol) using potassium fluoride (344 mg, 5.92 mmol) in dry DMF (10 mL) as per the procedure described in Step 4 of Intermediate 1 to yield 462 mg of the product as a solid. ¹H NMR (400 MHz, DMSO-d₆): δ 1.07 (t, J=4.8 Hz, 3H), 1.09-1.33 (m, 2H), 1.38-1.42 (m, 2H), 1.97-2.01 (m, 1H), 2.54 (q, J=7.6 Hz, 2H), 3.23 (t, J=10 Hz, 2H), 3.73 (d, J=7.2 Hz, 2H), 3.80-3.84 (m, 2H), 5.19 (s, 2H), 6.32 (s, 2H), 7.23-7.29 (m, 2H), 7.65-7.69 (m, 1H).

### Intermediate 208

2-(2-Chlorophenyl)-2-oxoethyl 5-amino-3-ethyl-1-((tetrahydro-2H-pyran-4-yl)methyl)-1H-pyrazole-4-carboxylate

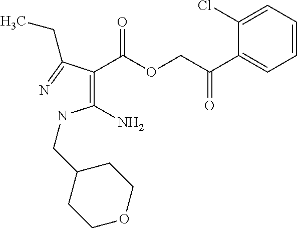 [SMILES: CCC(=N)/C(C(=O)OCC(=O)c1ccccc1Cl)=C(/N)NCC1CCOCC1]

The titled compound was prepared by the reaction of Step 2 intermediate 207 (1.0 g, 3.95 mmol) with 2-bromo-1-(2-chlorophenyl)ethanone (1.23 g, 4.74 mmol) using potassium fluoride (344 mg, 5.95 mmol) in dry DMF (13 mL) as per the procedure described in Step 4 of Intermediate 1 to yield 583 mg of the product as a solid. ¹H NMR (400 MHz, DMSO-d₆): δ 1.06 (t, J=5.6 Hz, 3H), 1.23-1.29 (m, 2H), 1.40-1.43 (m, 2H), 1.99-2.0 (m, 1H), 2.57 (q, J=7.6 Hz, 2H), 3.24 (t, J=10.0 Hz, 2H), 3.74 (d, J=7.2 Hz, 2H), 3.81-4.12 (m, 2H), 5.31 (s, 2H), 6.33 (s, 2H), 7.48-7.51 (m, 1H), 7.58-7.59 (m, 2H), 7.77-7.80 (m, 1H).

### Intermediate 209

2-(2,6-Difluorophenyl)-2-oxoethyl 5-amino-1-methyl-3-((tetrahydro-2H-pyran-4-yl)methyl)-1H-pyrazole-4-carboxylate

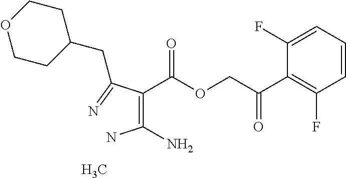

### Step 1: 5-Amino-1-methyl-3-((tetrahydro-2H-pyran-4-yl)methyl)-1H-pyrazole-4-carbonitrile

The titled compound was prepared by the reaction of 2-(1-methoxy-2-(tetrahydro-2H-pyran-4-yl)ethylidene)malononitrile (1.8 g, 8.73 mmol) with methylhydrazine sulphate (1.3 g, 8.73 mmol) using N,N-Diisopropylethylamine (3.0 mL, 17.47 mmol) in dry ethanol (20 mL) as per the procedure described in Step 1 of Intermediate 75 to yield 1.12 g of the product as yellow solid. ¹H NMR (400 MHz, CDCl₃): δ 1.26-1.51 (m, 2H), 1.63 (dd, J₁=2.0 Hz, J2 =10.4 Hz, 2H), 1.89-2.0 (m, 1H), 2.54 (d, J=7.6 Hz, 2H), 3.39 (dt, J₁=2.0 Hz, J₂=12.0 Hz, 2H), 3.39 (s, 3H), 3.96 (dd, J₁=2.8 Hz, J₂=9.6 Hz, 2H), 4.35-4.40 (br s, 2H).

### Step 2: 5-Amino-1-methyl-3-((tetrahydro-2H-pyran-4-yl)methyl)-1H-pyrazole-4-carboxylic acid

The titled compound was prepared by the reaction of Step 1 intermediate (1.10 g, 4.99 mnmol) with sodium hydroxide (1.2 g, 29.90 mmol) in water (12 mL) as per the procedure described in Step 2 of Intermediate 96 to yield 870 mg of the desired product as a solid. ¹H NMR (400 MHz, DMSO-d₆): δ 1.10-1.54 (m, 6H), 1.91-1.99 (m, 1H), 2.71-2.75 (m, 2H), 3.17-3.54 (m, 6H), 3.81 (d, J=9.6 Hz, 3H), 6.12 (s, 2H), 11.88 (br s, 1H).

### Step 3: 2-(2,6-Difluorophenyl)-2-oxoethyl 5-amino-1-methyl-3-((tetrahydro-2H-pyran-4-yl)methyl)-1H-pyrazole-4-carboxylate

The titled compound was prepared by the reaction of Step 2 intermediate (850 mg, 3.55 mmol) with 2-bromo-1-(2,6-difluorophenyl)ethanone (1.0 g, 4.26 mmol) using potassium fluoride (310 mg, 5.33 mmol) in dry DMF (9 mL) as per the procedure described in Step 4 of Intermediate 1 to yield 506 mg of the product as a yellow solid. ¹H NMR (400 MHz, DMSO-d₆): δ 1.13-1.23 (m, 2H), 1.47-1.50 (m, 2H), 1.79-1.85 (m, 1H), 2.47-2.51 (m, 2H), 3.20 (t, J=10.4 Hz, 2H), 3.47 (t, J=9.6 Hz, 3H), 3.79 (dd, J₁=2.4 Hz, J2 =7.2 Hz, 2H), 5.19 (s, 2H), 6.28 (s, 2H), 7.26 (t, J=8.8 Hz, 2H), 7.65-7.70 (m, 1H).

### Intermediate 210

2-(2-Chlorophenyl)-2-oxoethyl 5-amino-1-(2-(4,4-difluoropiperidin-1-yl)ethyl)-3-ethyl-1H-pyrazole-4-carboxylate

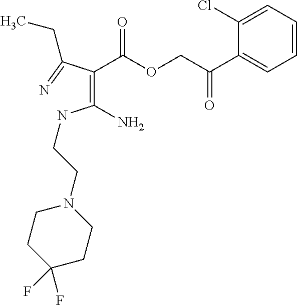 [SMILES: CCC(=N)/C(C(=O)OCC(=O)c1ccccc1Cl)=C(/N)NCCN1CCC(F)(F)CC1]

The titled compound was prepared by the reaction of Step 2 intermediate 205 (330 mg, 0.99 mmol) with 2-bromo-1-(2-chlorophenyl)ethanone (280 mg, 1.19 mmol) using potassium fluoride (90 mg, 1.49 mmol) in dry DMF (3.5 mL) as per the procedure described in Step 4 of Intermediate 1 to yield 330 mg of the product as a solid. ¹H NMR (400 MHz, DMSO-d₆): d 1.21 (t, J=7.6 Hz, 3H), 1.27-1.43 (m, 4H), 2.15 (br s, 2H), 2.73 (t, J=7.6 Hz, 2H), 2.83 (br s, 2H), 2.97 (s, 2H), 4.20 (br s, 2H), 5.36 (s, 2H), 6.27 (s, 2H), 7.37-7.41 (m, 1H), 7.48-7.50 (m, 2H), 7.67-7.69 (m, 1H).

### Intermediate 211

2-(2,6-Difluorophenyl)-2-oxoethyl 5-amino-3-ethyl-1-(2-(tetrahydro-2H-pyran-4-yl)ethyl)-1H-pyrazole-4-carboxylate

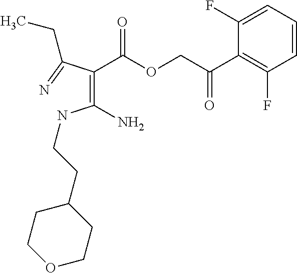 [SMILES: CCC(=N)/C(C(=O)OCC(=O)c1c(F)cccc1F)=C(/N)NCCC1CCOCC1]

### Step 1: Ethyl 5-amino-3-ethyl-1-(2-(tetrahydro-2H-pyran-4-yl)ethyl)-1H-pyrazole-4-carboxylate

The titled compound was prepared by the reaction of (E)-ethyl 2-cyano-3-methoxypent-2-enoate (3.2 g, 16.22 mmol) with ((tetrahydro-2H-pyran-4-yl)methyl)hydrazine (2.80 g, 19.47 mmol) using N,N-Diisopropylethylamine (5.6 mL, 32.44 mmol) in dry ethanol (32mL) as per the procedure described in Step 1 of Intermediate 75 to yield 4.03 g of the product as sticky oil. ¹H NMR (400 MHz, DMSO-d₆)): δ 1.09 (t, J=7.2 Hz, 4H), 1.25 (t, J=7.6 Hz, 4H), 1.34-1.36 (m, 1H), 1.55-1.60 (m, 4H), 2.59 (q, J=7.2 Hz, 2H), 3.23 (t, J=11.6 Hz, 2H), 3.80-3.87 (m, 4H), 4.16 (q, J=7.2 Hz, 2H), 6.17 (s, 2H).

### Step 2: 5-Amino-3-ethyl-1-(2-(tetrahydro-2H-pyran-4-yl)ethyl)-1H-pyrazole-4-carboxylic acid

The titled compound was prepared by the reaction of Step 1 intermediate (4.0 g, 13.60 mnmol) with potassium hydroxide (1.8 g, 27.10 mmol) in water (10 mL) and ethanol (40 mL) as per the procedure described in Step 3 of Intermediate 1 to yield 2.42 g of the desired product as a solid. ¹H NMR (300 MHz, DMSO-d₆): δ 1.07-1.24 (m, 5H), 1.44-1.60 (m, 5H), 2.58 (q, J=7.6 Hz, 2H), 3.21 (t, J=10.0 Hz, 2H), 3.82-3.85 (m, 4H), 6.14 (s, 2H), 11.68 (br s, 1H).

### Step 3: 2-(2,6-Difluorophenyl)-2-oxoethyl 5-amino-3-ethyl-1-(2-(tetrahydro-2H-pyran-4-yl)ethyl)-1H-pyrazole-4-carboxylate

The titled compound was prepared by the reaction of Step 2 intermediate 207 (1.0 g, 3.74 mmol) with 2-bromo-1-(2,6-difluorophenyl)ethanone (1.05 g, 4.49 mmol) using potassium fluoride (325 mg, 5.61 mmol) in dry DMF (10 mL) as per the procedure described in Step 4 of Intermediate 1 to yield 1.32 g of the product as a solid. ¹H NMR (400 MHz, DMSO-d₆): δ 1.07 (t, J=7.6 Hz, 3H), 1.15-1.61 (m, 7H), 2.56 (q, J=7.2 Hz, 2H), 3.16-3.28 (m, 2H), 3.79-3.89 (m, 4H), 5.20 (s, 2H), 6.29 (s, 2H), 7.26 (t, J=8.8 Hz, 2H), 7.64-7.71 (m, 1H).

### Intermediate 212

2-(2-Chlorophenyl)-2-oxoethyl 5-amino-3-ethyl-1-(2-(tetrahydro-2H-pyran-4-yl)ethyl)-1H-pyrazole-4-carboxylate

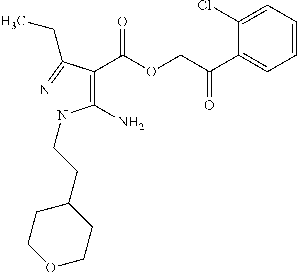 [SMILES: CCC(=N)/C(C(=O)OCC(=O)c1ccccc1Cl)=C(/N)NCCC1CCOCC1]

The titled compound was prepared by the reaction of Step 2 intermediate 211 (1.20 g, 4.49 mmol) with 2-bromo-1-(2-chlorophenyl)ethanone (1.60 g, 6.74 mmol) using potassium fluoride (520 mg, 8.98 mmol) in dry DMF (12 mL) as per the procedure described in Step 4 of Intermediate 1 to yield 980 mg of the product as a solid. ¹H NMR (400 MHz, DMSO-d₆): δ 1.09 (t, J=3.2 Hz, 3H), 1.15-1.62 (m, 7H), 2.57 (q, J=7.6 Hz, 2H), 3.19-3.29 (m, 2H), 3.80-3.90 (m, 4H), 5.32 (s, 2H), 6.30 (s, 2H), 7.47-7.52 (m, 1H), 7.58-7.60 (m, 2H), 7.77-7.80 (m, 1H).

### Intermediate 213

2-(2-Fluoro-3-(trifluoromethyl)phenyl)-2-oxoethyl 5-amino-3-ethyl-1-(2-(tetrahydro-2H-pyran-4-yl)ethyl)-1H-pyrazole-4-carboxylate

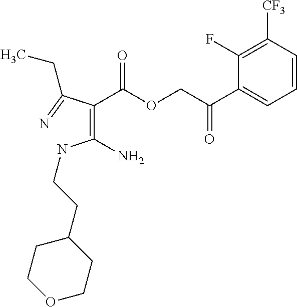 [SMILES: CCC(=N)/C(C(=O)OCC(=O)c1cccc(C(F)(F)F)c1F)=C(/N)NCCC1CCOCC1]

The titled compound was prepared by the reaction of Step 2 intermediate 211 (1.0 g, 3.74 mmol) with 2-bromo-1-(2-fluoro-3-(trifluoromethyl)phenyl)ethanone (1.6 g, 5.61 mmol) using potassium fluoride (434 mg, 7.49 mmol) in dry DMF (10 mL) as per the procedure described in Step 4 of Intermediate 1 to yield 1.35 g of the product as a yellow solid. ¹H NMR (400 MHz, DMSO-d₆): δ 1.10 (t, J=7.2 Hz, 3H), 1.13-1.63 (m, 7H), 2.61 (q, J=7.2 Hz, 2H), 3.16-3.28 (m, 2H), 3.79-3.90 (m, 4H), 5.39 (s, 2H), 6.31 (s, 2H), 7.59 (t, J=8.0 Hz, 1H), 8.10 (t, J=7.6 Hz, 1H), 8.20 (t, J=6.8 Hz, 1H).

### Intermediate 214

2-(2,6-Difluorophenyl)-2-oxoethyl 5-amino-3-(methoxymethyl)-1-((tetrahydro-2H-pyran-4-yl)methyl)-1H-pyrazole-4-carboxylate

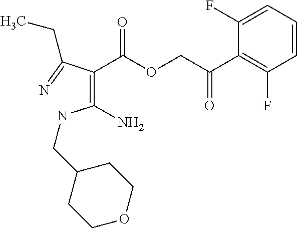 [SMILES: CCC(=N)/C(C(=O)OCC(=O)c1c(F)cccc1F)=C(/N)NCC1CCOCC1]

### Step 1: 5-Amino-3-(methoxymethyl)-1-((tetrahydro-2H-pyran-4-yl)methyl)-1H-pyrazole-4-carbonitrile

The titled compound was prepared by the reaction of 2-(1-(4-hydroxybutoxy)-2-methoxyethylidene)malononitrile (2.14 g, 10.25 mmol) with ((tetrahydro-2H-pyran-4-yl)methyl)hydrazine (1.6 g, 12.30 mmol) using N,N-Diisopropylethylamine (3.5 mL, 20.5 mmol) in dry ethanol (30 mL) as per the procedure described in Step 1 of Intermediate 75 to yield 1.72 g of the product as brown oil. ¹H NMR (400 MHz, DMSO-d₆)): d 1.20-1.28 (m, 2H), 1.38-1.41 (m, 2H), 1.95-2.01 (m, 1H), 3.17 (t, J=5.2 Hz, 2H), 3.22 (s, 3H), 3.75 (d, J=7.2 Hz, 2H), 3.81 (dd, J₁=2.8 Hz, J2 =11.2 Hz, 2H), 4.21 (s, 2H), 6.60 (s, 2H).

### Step 2: 5-Amino-3-(methoxymethyl)-1-(2-(tetrahydro-2H-pyran-4-yl)methyl)-1H-pyrazole-4-carboxylic acid

The titled compound was prepared by the reaction of Step 1 intermediate (2.2 g, 8.80 mnmol) with sodium hydroxide (3.52 g, 88.0 mmol) in water (44 mL) as per the procedure described in Step 2 of Intermediate 96 to yield 1.92 mg of the desired product as a brown sticky oil. ¹H NMR (400 MHz, DMSO-d₆): d 1.17-1.27 (m, 2H), 1.39-1.42 (m, 2H), 1.91-2.01 (m, 1H), 3.16-3.26 (m, 2H), 3.35 (s, 3H), 3.76-3.84 (m, 4H), 4.35 (s, 2H), 6.26 (s, 2H), 11.94 (s, 1H); ESI (m/z) 269 (M)⁺.

### Step 3: 2-(2,6-Difluorophenyl)-2-oxoethyl 5-amino-3-(methoxymethyl)-1-((tetrahydro-2H-pyran-4-yl)methyl)-1H-pyrazole-4-carboxylate

The titled compound was prepared by the reaction of Step 2 intermediate (1.9 g, 7.06 mmol) with 2-bromo-1-(2,6-difluorophenyl)ethanone (2.0 g, 8.47 mmol) using potassium fluoride (615 mg, 10.59 mmol) in dry DMF (20 mL) as per the procedure described in Step 4 of Intermediate 1 to yield 836 mg of the product as a solid. ¹H NMR (400 MHz, DMSO-d₆): d 1.20-1.30 (m, 2H), 1.40-1.42 (m, 2H), 1.91-2.01 (m, 1H), 3.17-3.26 (m, 5H), 3.78-4.14 (m, 4H), 4.35 (s, 2H), 5.21 (s, 2H), 6.41 (s, 2H), 7.24-7.29 (m. 2H), 7.64-7.71 (m, 1H); ESI (m/z) 424 (M)⁺.

### Intermediate 215

2-(2,6-difluorophenyl)-2-oxoethyl 5-amino-3-(methoxymethyl)-1-(2-(tetrahydro-2H-pyran-4-yl)ethyl)-1H-pyrazole-4-carboxylate

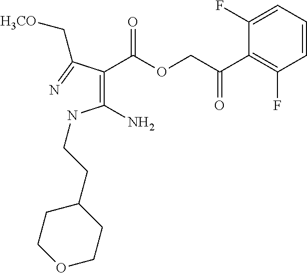 [SMILES: COCC(=N)/C(C(=O)OCC(=O)c1c(F)cccc1F)=C(/N)NCCC1CCOCC1]

### Step 1: 5-Amino-3-(methoxymethyl)-1-(2-(tetrahydro-2H-pyran-4-yl)ethyl)-1H-pyrazole-4-carbonitrile

The titled compound was prepared by the reaction of 2-(1,2-dimethoxyethylidene)malononitrile (2.8 g, 19.73 mmol) with (2-(tetrahydro-2H-pyran-4-yl)ethyl)hydrazine (2.8 g, 19.73 mmol) using N,N-Diisopropylethylamine (6.8 mL, 39.46 mmol) in dry ethanol (30 mL) as per the procedure described in Step 1 of Intermediate 75 to yield 2.0 g of the product as brown oil. ¹H NMR (400 MHz, DMSO-d₆)): δ 1.12-1.16 (m, 7H), 3.16-3.21 (m, 5H), 3.79-3.89 (m, 4H), 4.21 (s, 2H), 6.58 (s, 2H).

### Step 2: 5-amino-3-(methoxymethyl)-1-(2-(tetrahydro-2H-pyran-4-yl)ethyl)-1H-pyrazole-4-carboxylic acid

The titled compound was prepared by the reaction of Step 1 intermediate (2.0 g, 7.93 mnmol) with sodium hydroxide (3.17 g, 79.3 mmol) in water (20 mL) as per the procedure described in Step 2 of Intermediate 96 to yield 353 mg of the desired product as a brown sticky oil. ¹H NMR (300 MHz, DMSO-d₆): δ 1.15-1.60 (m, 7H), 1.91 (s, 3H), 3.17-3.28 (m, 4H), 3.80-3.89 (m, 2H), 4.35 (s, 2H), 6.22 (s, 2H), 11.94 (s, 1H).

### Step 3: 2-(2,6-Difluorophenyl)-2-oxoethyl 5-amino-3-(methoxymethyl)-1-(2-(tetrahydro-2H-pyran-4-yl)ethyl)-1H-pyrazole-4-carboxylate

The titled compound was prepared by the reaction of Step 2 intermediate (1.1 g, 3.88 mmol) with 2-bromo-1-(2,6-difluorophenyl)ethanone (1.37 g, 5.83 mmol) using potassium fluoride (450 mg, 7.77 mmol) in dry DMF (11 mL) as per the procedure described in Step 4 of Intermediate 1 to yield 353 mg of the product as a solid. ¹H NMR (400 MHz, DMSO-d₆): δ 1.15-1.61 (m, 7H), 3.16-3.27 (m, 5H), 3.82 (dd, J=2.8 Hz, 11.6 Hz, 2H), 3.92 (t, J=7.2 Hz, 2H), 4.33 (s, 2H), 5.21 (s, 2H), 6.38 (s, 2H), 7.27 (t, J=8.8 Hz, 2H), 7.64-7.72 (m, 1H).

### Intermediate 216

2-(2,6-difluorophenyl)-2-oxoethyl 5-amino-3-ethyl-1-(2-(methylsulfonyl)ethyl)-1H-pyrazole-4-carboxylate

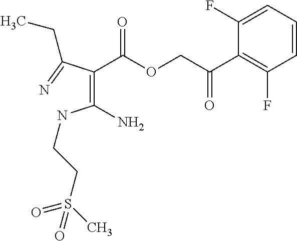 [SMILES: CCC(=N)/C(C(=O)OCC(=O)c1c(F)cccc1F)=C(/N)NCCS(C)(=O)=O]

### Step 1: Ethyl 5-amino-3-ethyl-1-(2-(methylsulfonyl)ethyl)-1H-pyrazole-4-carboxylate

The titled compound was prepared by the reaction of (Z)-ethyl 2-cyano-3-methoxypent-2-enoate (3.0 g, 15.21 mmol) with (2-(methylsulfonyl)ethyl)hydrazine (2.5 g, 18.10 mmol) using N,N-Diisopropylethylamine (5.24 mL, 30.40 mmol) in dry ethanol (30 mL) as per the procedure described in Step 1 of Intermediate 75 to yield 1.0 g of the product as brown oil. ¹H NMR (400 MHz, DMSO-d₆)): δ 1.11 (t, J=7.6 Hz, 3H), 1.25 (t, J=7.2 Hz, 3H), 2.61 (q, J=7.6 Hz, 2H), 2.97 (d, J=7.2 Hz, 3H), 3.53 (t, J=7.2 Hz, 2H), 4.17 (q, J=7.2 Hz, 2H), 4.26 (t, J=6.8 Hz, 2H), 6.34 (s, 2H).

### Step 2: 5-amino-3-ethyl-1-(2-(methylsulfonyl)ethyl)-1H-pyrazole-4-carboxylic acid

The titled compound was prepared by the reaction of Step 1 intermediate (1.0 g, 3.46 mnmol) with potassium hydroxide (0.387 g, 6.91 mmol) in water (5 mL) and ethanol (10 mL) as per the procedure described in Step 3 of Intermediate 1 to yield 430 mg of the desired product as a solid. ¹H NMR (300 MHz, DMSO-d₆): δ 1.10 (t, J=7.6 Hz, 3H), 2.60 (q, J=7.6 Hz, 2H), 2.96 (s, 3H), 3.53 (t, J=7.2 Hz, 2H), 4.25 (t, J=6.8 Hz, 2H), 6.30 (s, 2H), 12.01 (br s, 1H).

### Step 3: 2-(2,6-difluorophenyl)-2-oxoethyl 5-amino-3-ethyl-1-(2-(methylsulfonyl)ethyl)-1H-pyrazole-4-carboxylate

The titled compound was prepared by the reaction of Step 2 intermediate (200 mg, 0.765 mmol) with 2-bromo-1-(2,6-difluorophenyl)ethanone (215 mg, 0.918 mmol) using potassium fluoride (67 mg, 1.14 mmol) in dry DMF (2 mL) as per the procedure described in Step 4 of Intermediate 1 to yield 220 mg of the product as a solid. ¹H NMR (400 MHz, DMSO-d₆): δ 1.09 (t, J=7.6 Hz, 3H), 2.58 (q, J=7.6 Hz, 2H), 2.97 (s, 3H), 3.54 (t, J=7.2 Hz, 2H), 4.28 (t, J=6.8 Hz, 2H), 5.21 (s, 2H), 6.47 (s, 2H), 7.24-7.29 (m, 2H), 7.64-7.72 (m, 1H).

### EXAMPLES

The compounds of the present invention shown below are prepared from intermediates described above using synthetic schemes 1 to 20. General procedures for the preparation of compounds of present invention are given below.

### Method A:

### Example 1

### Synthesis of 6-(2-Chlorophenyl)-5-hydroxy-1,3-dimethyl-1,7-dihydro-4H-pyrazolo[3,4-b]pyridin-4-one

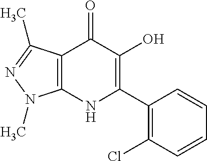 [SMILES: Cc1nn(C)c2[nH]c(-c3ccccc3Cl)c(O)c(=O)c12]

A mixture of 2-(2-Chlorophenyl)-2-oxoethyl 5-amino-1,3-dimethyl-1H-pyrazole-4-carboxylate (590 mg, 1.91 mmol) and polyphosphoric acid (6.0 mL) was heated to 120° C. for 3 h. The reaction mixture was cooled to RT and neutralized with 1 N sodium hydroxide. The reaction mixture was extracted with ethyl acetate (3×100 mL) and the organic layer was washed with water (100 mL). The organic layer was dried over anhydrous sodium sulfate and concentrated under reduced pressure. The residue obtained was purified by flash silica gel column chromatography to afford 112 mg of the titled product as a solid. ¹H NMR (300 MHz, DMSO-d₆): δ 2.52 (s, 3H), 3.80 (s, 3H), 7.45-7.60 (m, 4H), 7.89 (s, 1H), 11.62 (br s, 1H); APCI (m/z) 290 (M+H)⁺.

### Method B:

### Example 2

### Synthesis of 6-(2-Chlorophenyl)-5-hydroxy-3-methyl-1-(2-morpholinoethyl)-1H-pyrazolo[3,4-b]pyridin-4(7H)-one

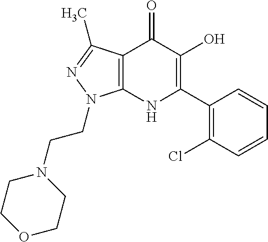 [SMILES: Cc1nn(CCN2CCOCC2)c2[nH]c(-c3ccccc3Cl)c(O)c(=O)c12]

A solution of 2-(2-Chlorophenyl)-2-oxoethyl 5-amino-3-methyl-1-(2-morpholinoethyl)-1H-pyrazole-4-carboxylate (Intermediate-91, 4.0 g, 9.85 mmol) in conc. sulfuric acid (30 mL) was stirred at 80° C. for 2 h. The reaction mixture was cooled to RT and quenched with ice cold water (35 mL). The precipitated solid was filtered and dried well to obtain 1.56 g of the desired product. ¹H NMR (300 MHz, DMSO-d₆): δ 2.39-2.52 (m, 7H), 2.69 (t, J=7.0 Hz, 2H), 3.16-3.41 (m, 4H), 4.27 (s, 2H), 7.49-7.63 (m, 4H), 7.91 (br s, 1H), 11.89 (br s, 1H); ESI (m/z) 389 (M+H)⁺.

The examples 3-190, 193-194, 198, 200, 202-213, 215 & 217-218 given in the Table-1 were prepared by following either of the above mentioned procedures. The structural formulas, chemical names, ¹H NMR and MS data are provided in Table-1.

TABLE 1
Structure, chemical name, ¹H NMR and MS data of the Examples 3-190, 193-194, 198, 200, 202-213, 215 & 217-218.

| Exam- | | Method/ | |
|---|---|---|---|
| ple No | Structure | Intermediate | Chemical name, ¹H NMR and MS data |
| 3 | 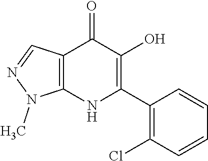 [SMILES: Cn1ncc2c(=O)c(O)c(-c3ccccc3Cl)[nH]c21] | A/Intermediate- 1 | 6-(2-Chlorophenyl)-5-hydroxy-1-methyl-1,7- dihydro-4H-pyrazolo[3,4-b]pyridin-4-one; ¹H NMR (300 MHz, DMSO-d₆): δ 3.89 (s, 3H), 7.40-7.52 (m, 4H), 7.96-8.07 (m, 2H), 11.83 (br s, 1H); APCI (m/z) 276 (M + H)⁺. |
| 4 | 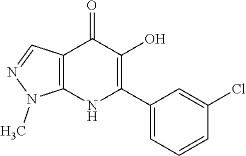 [SMILES: Cn1ncc2c(=O)c(O)c(-c3cccc(Cl)c3)[nH]c21] | A/Intermediate- 2 | 6-(3-Chlorophenyl)-5-hydroxy-1-methyl-1,7- dihydro-4H-pyrazolo[3,4-b]pyridin-4-one; ¹H NMR (300 MHz, DMSO-d₆): δ 3.95 (s, 3H), 7.42-7.58 (m, 2H), 7.99-8.09 (m, 2H), 8.51-8.60 (m, 2H), 11.45 (br s, 1H); ESI (m/z) 276 (M + H)⁺. |
| 5 | 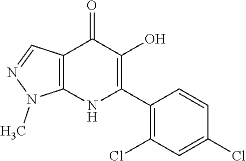 [SMILES: Cn1ncc2c(=O)c(O)c(-c3ccc(Cl)cc3Cl)[nH]c21] | A/Intermediate- 3 | 6-(2,4-Dichlorophenyl)-5-hydroxy-1-methyl- 1,7-dihydro-4H-pyrazolo[3,4-b]pyridin-4-one; ¹H NMR (300 MHz, DMF-d₆): δ 4.18 (s, 3H), 7.75-7.85 (m, 2H), 7.91-8.00 (m, 2H), 8.19-8.25 (m, 2H); APCI (m/z) 310 (M + H)⁺. |
| 6 | 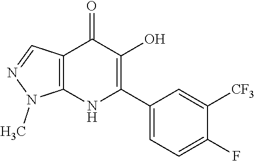 [SMILES: Cn1ncc2c(=O)c(O)c(-c3ccc(F)c(C(F)(F)F)c3)[nH]c21] | A/Intermediate- 4 | 6-[4-Fluoro-3-(trifluoromethyl)phenyl]-5- hydroxy-1-methyl-1,7-dihydro-4H- pyrazolo[3,4-b]pyridin-4-one; ¹H NMR (300 MHz, DMSO-d₆): δ 3.96 (s, 3H), 7.63 (t, J = 9.3 Hz, 1H), 8.05 (s, 1H), 8.31-8.40 (m, 2H); ESI (m/z) 328 (M + H)⁺. |
| 7 | 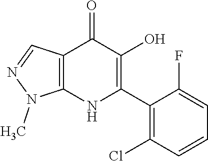 [SMILES: Cn1ncc2c(=O)c(O)c(-c3c(F)cccc3Cl)[nH]c21] | A/Intermediate- 5 | 6-(2-Chloro-6-fluorophenyl)-5-hydroxy-1- methyl-1,7-dihydro-4H-pyrazolo[3,4-b]pyridin- 4-one; ¹H NMR (300 MHz, DMSO-d₆): δ 3.89 (s, 3H), 7.25-7.50 (m, 3H), 7.95-8.06 (m, 1H), 8.39-8.48 (m, 1H), 11.43 (br s, 1H); APCI (m/z) 294 (M + H)⁺. |
| 8 | 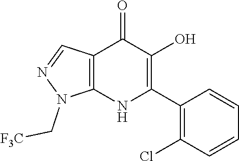 [SMILES: O=c1c(O)c(-c2ccccc2Cl)[nH]c2c1cnn2CC(F)(F)F] | A/Intermediate- 6 | 6-(2-Chlorophenyl)-5-hydroxy-1-(2,2,2- trifluoroethyl)-1,7-dihydro-4H-pyrazolo[3,4- b]pyridin-4-one; ¹H NMR (300 MHz, DMSO-d₆): δ 5.17 (q, J = 6.3 Hz, 2H), 7.37-7.45 (m, 2H), 7.49-7.58 (m, 2H), 8.26 (s, 1H), 8.47 (br s, 1H), 11.58 (br s, 1H); APCI (m/z) 344 (M + H)⁺. |
| 9 | 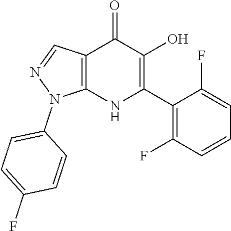 [SMILES: O=c1c(O)c(-c2c(F)cccc2F)[nH]c2c1cnn2-c1ccc(F)cc1] | A/Intermediate- 7 | 6-(2,6-Difluorophenyl)-1-(4-fluorophenyl)-5- hydroxy-1,7-dihydro-4H-pyrazolo[3,4- b]pyridin-4-one; ¹H NMR (300 MHz, DMSO- d₆): δ 7.21 (t, J = 7.8 Hz, 2H), 7.36 (t, J = 8.1 Hz, 2H), 7.52-7.58 (m, 1H), 8.24 (br s, 2H), 8.43 (s, 1H), 8.84 (br s, 1H), 11.83 (br s, 1H); APCI (m/z) 356 (M − H)⁻. |
| 10 | 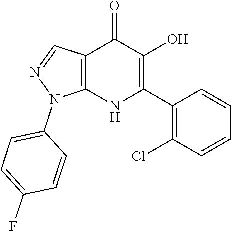 [SMILES: O=c1c(O)c(-c2ccccc2Cl)[nH]c2c1cnn2-c1ccc(F)cc1] | A/Intermediate- 8 | 6-(2-Chlorophenyl)-1-(4-fluorophenyl)-5- hydroxy-1,7-dihydro-4H-pyrazolo[3,4- b]pyridin-4-one; ¹H NMR (300 MHz, DMSO- d₆): δ 7.31-7.54 (m, 5H), 8.25-8.27 (m, 2H), 8.40 (s, 1H), 8.62 (br s, 1H), 11.43 (br s, 1H); APCI (m/z) 356 (M + H)⁺. |
| 11 | 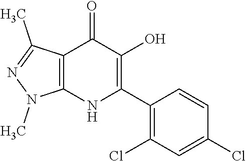 [SMILES: Cc1nn(C)c2[nH]c(-c3ccc(Cl)cc3Cl)c(O)c(=O)c12] | A/Intermediate- 10 | 6-(2,4-Dichlorophenyl)-5-hydroxy-1,3- dimethyl-1,7-dihydro-4H-pyrazolo[3,4- b]pyridin-4-one; ¹H NMR (300 MHz, DMSO- d₆): δ 2.49 (s, 3H), 3.76 (s, 3H), 7.51-7.60 (m, 2H), 7.82 (s, 1H), 8.03 (s, 1H), 11.57 (br s, 1H); APCI (m/z) 324 (M)⁺. |
| 12 | 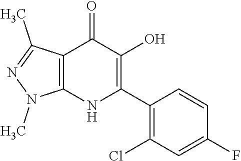 [SMILES: Cc1nn(C)c2[nH]c(-c3ccc(F)cc3Cl)c(O)c(=O)c12] | A/Intermediate- 11 | 6-(2-Chloro-4-fluorophenyl)-5-hydroxy-1,3- dimethyl-1,7-dihydro-4H-pyrazolo[3,4- b]pyridin-4-one; ¹H NMR (300 MHz, DMSO- d₆): δ 2.49 (s, 3H), 3.77 (s, 3H), 7.32-7.42 (m, 1H), 7.51-7.67 (m, 1H), 7.90-7.99 (m, 1H), 11.58 (br s, 1H); APCI (m/z) 308 (M + H)⁺. |
| 13 | 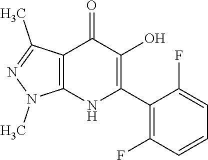 [SMILES: Cc1nn(C)c2[nH]c(-c3c(F)cccc3F)c(O)c(=O)c12] | A/Intermediate- 12 | 6-(2,6-Difluorophenyl)-5-hydroxy-1,3- dimethyl-1,7-dihydro-4H-pyrazolo[3,4- b]pyridin-4-one; ¹H NMR (300 MHz, DMSO- d₆): δ 2.46 (s, 3H), 3.77 (s, 3H), 7.15-7.35 (m, 2H), 7.41-7.66 (m, 1H), 8.23 (br s, 1H), 11.70 (br s, 1H); APCI (m/z) 292 (M + H)⁺. |
| 14 | 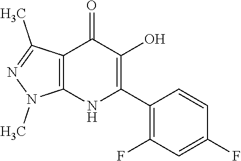 [SMILES: Cc1nn(C)c2[nH]c(-c3ccc(F)cc3F)c(O)c(=O)c12] | A/Intermediate- 13 | 6-(2,4-Difluorophenyl)-5-hydroxy-1,3- dimethyl-1,7-dihydro-4H-pyrazolo[3,4- b]pyridin-4-one; ¹H NMR (300 MHz, DMSO- d₆): δ 2.47 (s, 3H), 3.78 (s, 3H), 7.18-7.28 (m, 1H), 7.30-7.48 (m, 1H), 7.52-7.70 (m, 1H), 8.03 (br s, 1H), 11.56 (br s, 1H); APCI (m/z) 292 (M + H)⁺. |
| 15 | 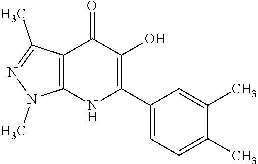 [SMILES: Cc1ccc(-c2[nH]c3c(c(C)nn3C)c(=O)c2O)cc1C] | A/Intermediate- 14 | 6-(3,4-Dimethylphenyl)-5-hydroxy-1,3- dimethyl-1,7-dihydro-4H-pyrazolo[3,4- b]pyridin-4-one; ¹H NMR (300 MHz, DMSO- d₆): δ 2.30 (s, 6H), 2.45 (s, 3H), 3.83 (s, 3H), 7.27 (d, J = 7.5 Hz, 1H), 7.41-7.47 (m, 2H), 7.78 (br s, 1H), 11.25 (br s, 1H); APCI (m/z) 284 (M + H)⁺. |
| 16 | 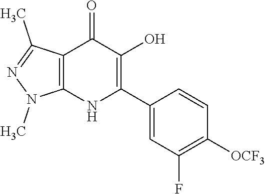 [SMILES: Cc1nn(C)c2[nH]c(-c3ccc(OC(F)(F)F)c(F)c3)c(O)c(=O)c12] | A/Intermediate- 15 | 6-[3-Fluoro-4-(trifluoromethoxy)phenyl]-5- hydroxy-1,3-dimethyl-1,7-dihydro-4H- pyrazolo[3,4-b]pyridin-4-one; ¹H NMR (300 MHz, DMF-d₇): δ 2.56 (s, 3H), 3.94 (s, 3H), 7.68 (t, J = 8.7 Hz, 1H), 7.80-8.30 (m, 2H), 11.25 (br s, 1H); APCI (m/z) 358 (M + H)⁺. |
| 17 | 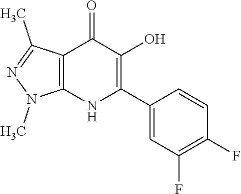 [SMILES: Cc1nn(C)c2[nH]c(-c3ccc(F)c(F)c3)c(O)c(=O)c12] | A/Intermediate- 16 | 6-(3,4-Difluorophenyl)-5-hydroxy-1,3- dimethyl-1,7-dihydro-4H-pyrazolo[3,4- b]pyridin-4-one; ¹H NMR (300 MHz, DMSO-d₆): δ 2.55 (s, 3H), 3.94 (s, 3H), 7.56- 7.60 (m, 2H), 7.75-7.92 (m, 3H), 11.32 (br s, 1H); APCI (m/z) 292 (M + H)⁺. |
| 18 | 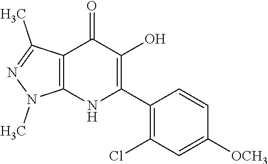 [SMILES: COc1ccc(-c2[nH]c3c(c(C)nn3C)c(=O)c2O)c(Cl)c1] | A/Intermediate- 17 | 6-(2-Chloro-4-methoxyphenyl)-5-hydroxy-1,3- dimethyl-1,7-dihydro-4H-pyrazolo[3,4- b]pyridin-4-one; ¹H NMR (300 MHz, DMSO-d₆): δ 2.47 (s, 3H), 3.77 (s, 3H), 3.84 (s, 3H), 7.05 (d, J = 8.1 Hz, 1H), 7.19 (s, 1H), 7.42 (d, J = 8.1 Hz, 1H), 7.79 (br s, 1H), 11.52 (br s, 1H); APCI (m/z) 320 (M + H)⁺. |
| 19 | 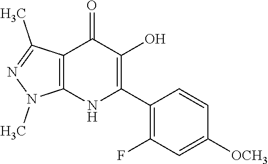 [SMILES: COc1ccc(-c2[nH]c3c(c(C)nn3C)c(=O)c2O)c(F)c1] | A/Intermediate- 18 | 6-(2-Fluoro-4-methoxyphenyl)-5-hydroxy-1,3- dimethyl-1,7-dihydro-4H-pyrazolo[3,4- b]pyridin-4-one; ¹H NMR (300 MHz, DMSO-d₆): δ 2.45 (s, 3H), 3.78 (s, 3H), 3.83 (s, 3H), 6.90- 7.02 (m, 2H), 7.45 (t, J = 8.4 Hz, 1H), 7.85 (s, 1H), 11.49 (br s, 1H); ESI (m/z) 304 (M + H)⁺. |
| 20 | 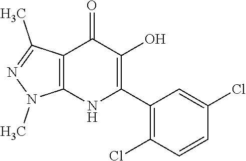 [SMILES: Cc1nn(C)c2[nH]c(-c3cc(Cl)ccc3Cl)c(O)c(=O)c12] | A/Intermediate- 19 | 6-(2,5-Dichlorophenyl)-5-hydroxy-1,3- dimethyl-1,7-dihydro-4H-pyrazolo[3,4-b]pyridin- 4-one; ¹H NMR (300 MHz, DMSO-d₆): δ 2.47 (s, 3H), 3.77 (s, 3H), 7.55-7.66 (m, 3H), 8.08 (s, 1H), 11.60 (br s, 1H); ESI (m/z) 326 (M)⁺. |
| 21 | 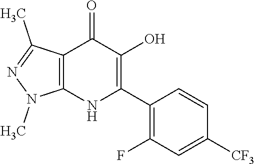 | A/Intermediate- 20 | 6-[2-Fluoro-4-(trifluoromethyl)phenyl]-5- hydroxy-1,3-dimethyl-1,7-dihydro-4H- pyrazolo[3,4-b]pyridin-4-one; ¹H NMR (300 MHz, DMSO-d₆, D₂O exchange): δ 2.47 (s, 3H), 3.76 (s, 3H), 7.72-7.81 (m, 3H); ESI (m/z) 339 (M − H)⁻. |
| 22 | 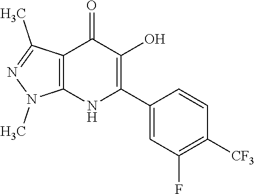 [SMILES: Cc1nn(C)c2[nH]c(-c3ccc(C(F)(F)F)c(F)c3)c(O)c(=O)c12] | A/Intermediate- 21 | 6-[3-Fluoro-4-(trifluoromethyl)phenyl]-5- hydroxy-1,3-dimethyl-1,7-dihydro-4H- pyrazolo[3,4-b]pyridin-4-one; ¹H NMR (300 MHz, DMF-d₇): δ 2.57 (s, 3H), 3.95 (s, 3H), 7.92-8.00 (m, 1H), 8.03 (s, 1H); ESI (m/z) 342 (M + H)⁺. |
| 23 | 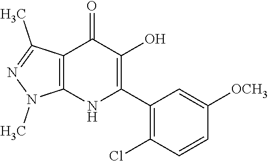 [SMILES: COc1ccc(Cl)c(-c2[nH]c3c(c(C)nn3C)c(=O)c2O)c1] | A/Intermediate- 22 | 6-(2-Chloro-5-methoxyphenyl)-5-hydroxy-1,3- dimethyl-1,7-dihydro-4H-pyrazolo[3,4- b]pyridin-4-one; ¹H NMR (300 MHz, DMSO- d₆): δ 2.45 (s, 3H), 3.76 (s, 3H), 3.78 (s, 3H), 7.07 (br s, 2H), 7.45-7.49 (m, 1H), 7.85 (br s, 1H), 11.57 (br s, 1H); ESI (m/z) 318 (M − H)⁻. |
| 24 | 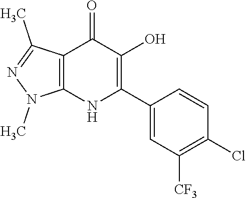 [SMILES: Cc1nn(C)c2[nH]c(-c3ccc(Cl)c(C(F)(F)F)c3)c(O)c(=O)c12] | A/Intermediate- 23 | 6-[3-Chloro-3-(trifluoromethyl)phenyl]-5- hydroxy-1,3-dimethyl-1,7-dihydro-4H- pyrazolo[3,4-b]pyridin-4-one; ¹H NMR (300 MHz, DMSO-d₆): δ 3.85 (s, 6H), 3.76 (s, 3H), 3.78 (s, 3H), 7.07 (br s, 2H), 7.45-7.49 (m, 1H), 7.85 (br s, 1H), 11.57 (br s, 1H); ESI (m/z) 358 (M + H)⁺. |
| 25 | 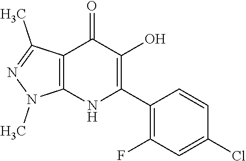 [SMILES: Cc1nn(C)c2[nH]c(-c3ccc(Cl)cc3F)c(O)c(=O)c12] | A/Intermediate- 24 | 6-(4-Chloro-2-fluorophenyl)-5-hydroxy-1,3- dimethyl-1,7-dihydro-4H-pyrazolo[3,4- b]pyridin-4-one; ¹H NMR (300 MHz, DMSO- d₆): δ 2.46 (s, 3H), 3.77 (s, 3H), 7.46-7.49 (m, 1H), 7.60-7.64 (m, 2H), 8.14 (s, 1H), 11.57 (br s, 1H); ESI (m/z) 308 (M + H)⁺. |
| 26 | 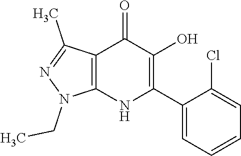 [SMILES: CCn1nc(C)c2c(=O)c(O)c(-c3ccccc3Cl)[nH]c21] | A/Intermediate- 25 | 6-(2-Chlorophenyl)-1-ethyl-5-hydroxy-3- methyl-1,7-dihydro-4H-pyrazolo[3,4-b]pyridin- 4-one; ¹H NMR (300 MHz, DMSO-d₆): δ 1.26 (t, J = 6.9 Hz, 3H), 2.47 (d, J = 6.3 Hz, 3H), 4.16 (q, J = 6.9 Hz, 2H), 7.48-7.53 (m, 3H), 7.61 (d, J = 7.8 Hz, 1H), 7.86 (br s, 1H), 11.56 (br s, 1H); APCI (m/z) 304 (M + H)⁺. |
| 27 | 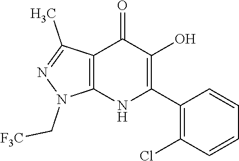 [SMILES: Cc1nn(CC(F)(F)F)c2[nH]c(-c3ccccc3Cl)c(O)c(=O)c12] | A/Intermediate- 26 | 6-(2-Chlorophenyl)-5-hydroxy-3-methyl-1- (2,2,2-trifluoroethyl)-1,7-dihydro-4H- pyrazolo[3,4-b]pyridin-4-one; ¹H NMR (300 MHz, DMSO-d₆): δ 2.56 (s, 3H), 5.09 (br s, 2H), 7.42-7.63 (m, 4H), 8.13 (s, 1H), 11.85 (br s, 1H); APCI (m/z) 358 (M + H)⁺. |
| 28 | 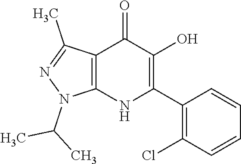 [SMILES: Cc1nn(C(C)C)c2[nH]c(-c3ccccc3Cl)c(O)c(=O)c12] | A/Intermediate- 27 | 6-(2-Chlorophenyl)-5-hydroxy-3-methyl-1- (propan-2-yl)-1,7-dihydro-4H-pyrazolo[3,4- b]pyridin-4-one; ¹H NMR (300 MHz, DMSO- d₆): δ 1.36 (d, J = 6.3 Hz, 6H), 2.49 (s, 3H), 4.74- 4.78 (m, 1H), 7.42-7.55 (m, 3H), 7.62 (d, J = 7.8 Hz, 1H), 7.87 (s, 1H), 11.50 (br s, 1H); APCI (m/z) 318 (M + H)⁺. |
| 29 | 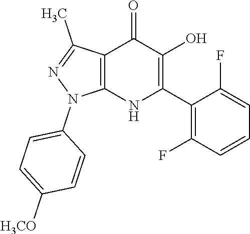 [SMILES: COc1ccc(-n2nc(C)c3c(=O)c(O)c(-c4c(F)cccc4F)[nH]c32)cc1] | A/Intermediate- 28 | 6-(2,6-Difluorophenyl)-5-hydroxy-1-(4- methoxyphenyl)-3-methyl-1,7-dihydro-4H- pyrazolo[3,4-b]pyridin-4-one; ¹H NMR (300 MHz, DMSO-d₆): δ 2.64 (s, 3H), 3.76 (s, 3H), 7.04 (d, J = 8.4 Hz, 2H), 7.21 (t, J = 7.8 Hz, 2H), 7.52-7.57 (m, 1H), 8.02 (d, J = 7.8 Hz, 2H), 8.40 (br s, 1H), 11.71 (br s, 1H); APCI (m/z) 384 (M + H)⁺. |
| 30 | 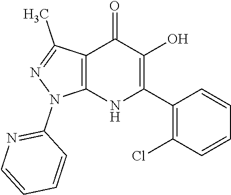 [SMILES: Cc1nn(-c2ccccn2)c2[nH]c(-c3ccccc3Cl)c(O)c(=O)c12] | A/Intermediate- 29 | 6-(2-Chlorophenyl)-5-hydroxy-3-methyl-1- (pyridin-2-yl)-1,7-dihydro-4H-pyrazolo[3,4- b]pyridin-4-one; ¹H NMR (300 MHz, DMSO- d₆): δ 2.62 (s, 3H), 7.29-7.32 (m, 1H), 7.51-7.62 (m, 4H), 7.83-7.88 (m, 1H), 8.00-8.05 (m, 1H), 8.45 (br s, 2H), 11.41 (br s, 1H); ESI (m/z) 353 (M + H)⁺. |
| 31 | 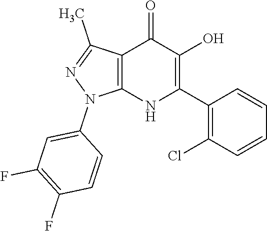 [SMILES: Cc1nn(-c2ccc(F)c(F)c2)c2[nH]c(-c3ccccc3Cl)c(O)c(=O)c12] | A/Intermediate- 30 | 6-(2-Chlorophenyl)-1-(3,4-difluorophenyl)-5- hydroxy-3-methyl-1,7-dihydro-4H- pyrazolo[3,4-b]pyridin-4-one; ¹H NMR (300 MHz, DMSO-d₆): δ 2.64 (s, 3H), 7.46-7.57 (m, 5H), 8.11-8.13 (m, 1H), 8.29-8.32 (m, 1H), 11.18 (br s, 1H); ESI (m/z) 388 (M + H)⁺. |
| 32 | 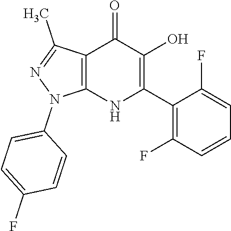 [SMILES: Cc1nn(-c2ccc(F)cc2)c2[nH]c(-c3c(F)cccc3F)c(O)c(=O)c12] | A/Intermediate- 31 | 6-(2,6-Difluorophenyl)-1-(4-fluorophenyl)-5- hydroxy-3-methyl-1,7-dihydro-4H- pyrazolo[3,4-b]pyridin-4-one; ¹H NMR (300 MHz, DMSO-d₆): δ 2.64 (s, 3H), 7.21 (t, J = 7.5 Hz, 2H), 7.31 (t, J = 7.5 Hz, 2H), 7.52-7.55 (m, 1H), 8.20 (br s, 2H), 8.31 (s, 1H), 10.93 (br s, 1H); ESI (m/z) 372 (M + H)⁺. |
| 33 | 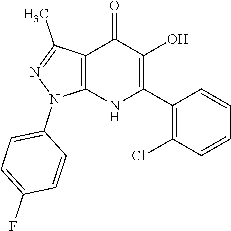 [SMILES: Cc1nn(-c2ccc(F)cc2)c2[nH]c(-c3ccccc3Cl)c(O)c(=O)c12] | A/Intermediate- 32 | 6-(2-Chlorophenyl)-1-(4-fluorophenyl)-5- hydroxy-3-methyl-1,7-dihydro-4H- pyrazolo[3,4-b]pyridin-4-one; ¹H NMR (300 MHz, DMSO-d₆): δ 2.63 (s, 3H), 7.26-7.30 (m, 2H), 7.40-7.65 (m, 6H), 8.21 (br s, 1H); APCI (m/z) 368 (M + H)⁺. |
| 34 | 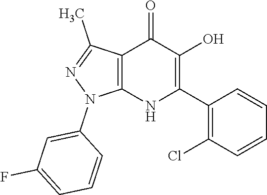 [SMILES: Cc1nn(-c2cccc(F)c2)c2[nH]c(-c3ccccc3Cl)c(O)c(=O)c12] | A/Intermediate- 33 | 6-(2-Chlorophenyl)-1-(3-fluorophenyl)-5- hydroxy-3-methyl-1,7-dihydro-4H- pyrazolo[3,4-b]pyridin-4-one; ¹H NMR (300 MHz, DMSO-d₆): δ 2.64 (s, 3H), 7.01 (s, 1H), 7.46-7.56 (m, 5H), 8.13 (br s, 2H), 8.44 (br s, 1H), 10.92 (br s, 1H); ESI (m/z) 368 (M + H)⁺. |
| 35 | 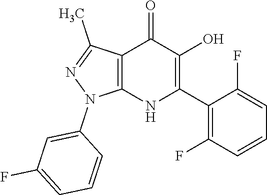 [SMILES: Cc1nn(-c2cccc(F)c2)c2[nH]c(-c3c(F)cccc3F)c(O)c(=O)c12] | A/Intermediate- 34 | 6-(2,6-Difluorophenyl)-1-(3-fluorophenyl)-5- hydroxy-3-methyl-1,7-dihydro-4H- pyrazolo[3,4-b]pyridin-4-one; ¹H NMR (300 MHz, DMSO-d₆): δ 2.65 (s, 3H), 7.00-7.06 (m, 1H), 7.23 (t, J = 8.4 Hz, 2H), 7.48- 7.55 (m, 2H), 8.09-8.12 (m, 2H), 8.68 (br s, 1H), 11.01 (br s, 1H); ESI (m/z) 372 (M + H)⁺. |
| 36 | 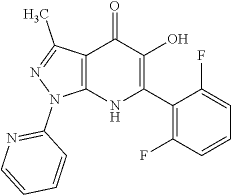 [SMILES: Cc1nn(-c2ccccn2)c2[nH]c(-c3c(F)cccc3F)c(O)c(=O)c12] | A/Intermediate- 35 | 6-(2,6-Difluorophenyl)-5-hydroxy-3-methyl-1- (pyridin-2-yl)-1,7-dihydro-4H-pyrazolo[3,4- b]pyridin-4-one; ¹H NMR (300 MHz, DMSO- d₆): δ 2.62 (s, 3H), 7.23-7.29 (m, 3H), 7.62-7.65 (m, 1H), 7.84 (d, J = 8.4 Hz, 1H), 8.01 (t, J = 7.2 Hz, 1H), 8.45 (br s, 1H), 8.70 (s, 1H), 11.68 (br s, 1H); ESI (m/z) 355 (M + H)⁺. |
| 37 | 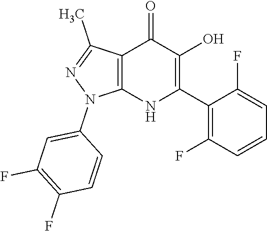 | A/Intermediate- 36 | 6-(2,6-Difluorophenyl)-1-(3,4-difluorophenyl)- 5-hydroxy-3-methyl-1,7-dihydro-4H- pyrazolo[3,4-b]pyridin-4-one; ¹H NMR (300 MHz, DMSO-d₆): δ 2.64 (s, 3H), 7.23 (t, J = 7.8 Hz, 2H), 7.52-7.57 (m, 2H), 8.07-8.09 (m, 1H), 8.20-8.25 (m, 1H), 8.64 (br s, 1H), 10.90 (br s, 1H). ESI (m/z) 355 (M + H)⁺. |
| 38 | 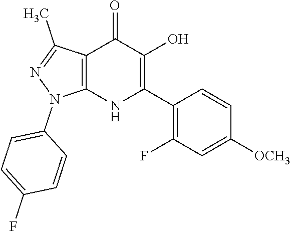 [SMILES: COc1ccc(-c2[nH]c3c(c(C)nn3-c3ccc(F)cc3)c(=O)c2O)c(F)c1] | A/Intermediate- 37 | 6-(2-Fluoro-4-methoxyphenyl)-1-(4- fluorophenyl)-5-hydroxy-3-methyl-1,7-dihydro- 4H-pyrazolo[3,4-b]pyridin-4-one; ¹H NMR (300 MHz, DMSO-d₆): δ 2.48 (s, 3H), 3.82 (s, 3H), 6.89-6.94 (m, 2H), 7.30 (t, J = 9.0 Hz, 2H), 7.45- 7.50 (m, 1H), 8.25-8.28 (m, 2H); APCI (m/z) 384 (M + H)⁺. |
| 39 | 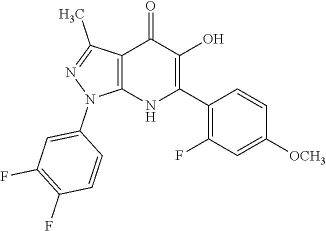 [SMILES: COc1ccc(-c2[nH]c3c(c(C)nn3-c3ccc(F)c(F)c3)c(=O)c2O)c(F)c1] | A/Intermediate- 38 | 1-(3,4-Difluorophenyl)-6-(2-fluoro-4- methoxyphenyl)-5-hydroxy-3-methyl-1,7- dihydro-4H-pyrazolo[3,4-b]pyridin-4-one; ¹H NMR (300 MHz, DMSO-d₆): δ 2.62 (s, 3H), 3.83 (s, 3H), 6.91 (t, J = 9.9 Hz, 2H), 7.52 (t, J = 9.0 Hz, 2H), 8.10-8.20 (m, 1H), 8.27-8.42 (m, 2H), 10.90 (br s, 1H); ESI (m/z) 402 (M + H)⁺. |
| 40 | 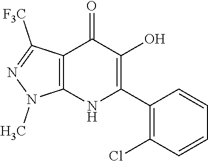 [SMILES: Cn1nc(C(F)(F)F)c2c(=O)c(O)c(-c3ccccc3Cl)[nH]c21] | A/Intermediate- 39 | 6-(2-Chlorophenyl)-5-hydroxy-1-methyl-3- (trifluoromethyl)-1,7-dihydro-4H-pyrazolo[3,4- b]pyridin-4-one; ¹H NMR (300 MHz, DMSO- d₆): δ 3.98 (s, 3H), 7.41-7.55 (m, 4H), 8.33 (br s, 1H), 12.17 (br s, 1H); APCI (m/z) 344 (M + H)⁺. |
| 41 | 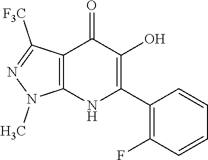 | A/Intermediate- 40 | 6-(2-Fluorophenyl)-5-hydroxy-1-methyl-3- (trifluoromethyl)-1,7-dihydro-4H-pyrazolo[3,4- b]pyridin-4-one; ¹H NMR (300 MHz, DMSO-d₆): δ 3.99 (s, 3H), 7.29-7.40 (m, 2H), 7.50-7.60 (m, 2H), 8.39 (br s, 1H), 12.05 (br s, 1H); APCI (m/z) 344 (M + H)⁺. |
| 42 | 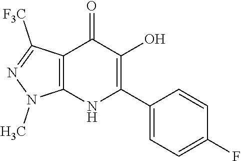 [SMILES: Cn1nc(C(F)(F)F)c2c(=O)c(O)c(-c3ccc(F)cc3)[nH]c21] | A/Intermediate- 41 | 6-(4-Fluorophenyl)-5-hydroxy-1-methyl-3- (trifluoromethyl)-1,7-dihydro-4H-pyrazolo[3,4- b]pyridin-4-one; ¹H NMR (300 MHz, DMSO-d₆): δ 4.03 (s, 3H), 7.35 (t, J = 6.3 Hz, 2H), 7.85-7.95 (m, 2H), 11.10 (br s, 1H); APCI (m/z) 328 (M + H)⁺. |
| 43 | 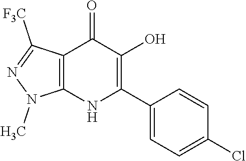 [SMILES: Cn1nc(C(F)(F)F)c2c(=O)c(O)c(-c3ccc(Cl)cc3)[nH]c21] | A/Intermediate- 42 | 6-(4-Chlorophenyl)-5-hydroxy-1-methyl-3- (trifluoromethyl)-1,7-dihydro-4H-pyrazolo[3,4- b]pyridin-4-one; ¹H NMR (300 MHz, DMSO- d₆): δ 4.03 (s, 3H), 7.59 (d, J = 8.1 Hz, 2H), 7.89- 7.95 (m, 2H), 8.40 (br s, 1H), 11.40 (br s, 1H); APCI (m/z) 342 (M + H)⁺. |
| 44 | 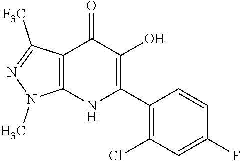 [SMILES: Cn1nc(C(F)(F)F)c2c(=O)c(O)c(-c3ccc(F)cc3Cl)[nH]c21] | A/Intermediate- 43 | 6-(2-Chloro-4-fluorophenyl)-5-hydroxy-1- methyl-3-(trifluoromethyl)-1,7-dihydro-4H pyrazolo[3,4-b]pyridin-4-one; ¹H NMR (300 MHz, DMSO-d₆): δ 3.98 (s, 3H), 7.31-7.40 (m, 1H), 7.55-7.67 (m, 2H), 8.31 (br s, 1H), 12.10 (br s, 1H); APCI (m/z) 362 (M + H)⁺. |
| 45 | 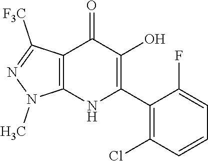 [SMILES: Cn1nc(C(F)(F)F)c2c(=O)c(O)c(-c3c(F)cccc3Cl)[nH]c21] | A/Intermediate- 44 | 6-(2-Chloro-6-fluorophenyl)-5-hydroxy-1- methyl-3-(trifluoromethyl)-1,7-dihydro-4H- pyrazolo[3,4-b]pyridin-4-one; ¹H NMR (300 MHz, DMSO-d₆): δ 3.98 (s, 3H), 7.41-7.61 (m, 3H), 8.59 (br s, 1H), 12.30 (br s, 1H); APCI (m/z) 362 (M + H)⁺. |
| 46 | 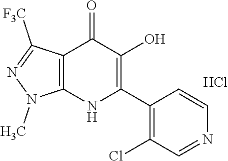 [SMILES: Cn1nc(C(F)(F)F)c2c(=O)c(O)c(-c3ccncc3Cl)[nH]c21 ; Cl] | A/Intermediate- 45 | 6-(3-Chloropyridin-4-yl)-5-hydroxy-1-methyl- 3-(trifluoromethyl)-1,7-dihydro-4H- pyrazolo[3,4-b]pyridin-4-one hydrochloride; ¹H NMR (300 MHz, DMSO-d₆): δ 4.00 (s, 3H), 7.05-7.39 (m, 3H), 7.56-7.60 (m, 1H), 8.65-8.68 (m, 1H), 8.80 (s, 1H); APCI (m/z) 345 (M + H)⁺. |
| 47 | 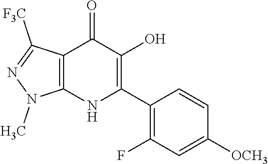 [SMILES: COc1ccc(-c2[nH]c3c(c(C(F)(F)F)nn3C)c(=O)c2O)c(F)c1] | A/Intermediate- 46 | 6-(2-Fluoro-4-methoxyphenyl)-5-hydroxy-1- methyl-3-(trifluoromethyl)-1,7-dihydro-4H- pyrazolo[3,4-b]pyridin-4-one; ¹H NMR (300 MHz, DMSO-d₆): δ 3.84 (s, 3H), 3.98 (s, 3H), 6.90-6.95 (m, 2H), 7.47 (t, J = 8.4 Hz, 1H), 8.23- 8.28 (m, 1H), 12.04 (br s, 1H); APCI (m/z) 358 (M + H)⁺. |
| 48 | 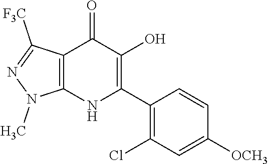 [SMILES: COc1ccc(-c2[nH]c3c(c(C(F)(F)F)nn3C)c(=O)c2O)c(Cl)c1] | A/Intermediate- 47 | 6-(2-Chloro-4-methoxyphenyl)-5-hydroxy-1- methyl-3-(trifluoromethyl)-1,7-dihydro-4H- pyrazolo[3,4-b]pyridin-4-one; ¹H NMR (300 MHz, DMSO-d₆): δ 3.84 (s, 3H), 3.97 (s, 3H), 7.04-7.06 (m, 1H), 7.19-7.22 (m, 1H), 7.40-7.44 (m, 1H), 8.23 (br s, 1H), 12.1 (br s, 1H); ESI (m/z) 374 (M + H)⁺. |
| 49 | 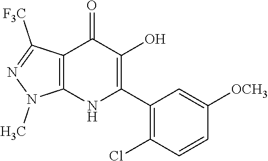 [SMILES: COc1ccc(Cl)c(-c2[nH]c3c(c(C(F)(F)F)nn3C)c(=O)c2O)c1] | A/Intermediate- 48 | 6-(2-Chloro-5-methoxyphenyl)-5-hydroxy-1- methyl-3-(trifluoromethyl)-1,7-dihydro-4H- pyrazolo[3,4-b]pyridin-4-one; ¹H NMR (300 MHz, DMSO-d₆): δ 3.79 (s, 3H), 3.98 (s, 3H), 7.05-7.10 (m, 2H), 7.46-7.50 (m, 1H), 8.29 (br s, 1H), 12.16 (br s, 1H); ESI (m/z) 374 (M + H)⁺. |
| 50 | 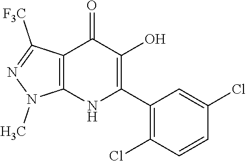 [SMILES: Cn1nc(C(F)(F)F)c2c(=O)c(O)c(-c3cc(Cl)ccc3Cl)[nH]c21] | A/Intermediate- 49 | 6-(2,5-Dichlorophenyl)-5-hydroxy-1-methyl-3- (trifluoromethyl)-1,7-dihydro-4H-pyrazolo[3,4- b]pyridin-4-one; ¹H NMR (300 MHz, DMSO- d₆): δ 3.98 (s, 3H), 7.58-7.62 (m, 3H); APCI (m/z) 379 (M + H)⁺. |
| 51 | 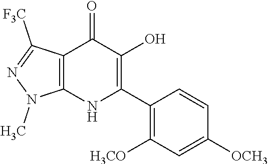 [SMILES: COc1ccc(-c2[nH]c3c(c(C(F)(F)F)nn3C)c(=O)c2O)c(OC)c1] | A/Intermediate- 50 | 6-(2,4-Dimethoxyphenyl)-5-hydroxy-1-methyl- 3-(trifluoromethyl)-1,7-dihydro-4H- pyrazolo[3,4-b]pyridin-4-one; ¹H NMR (300 MHz, DMSO-d₆): δ 3.64 (s, 3H), 3.83 (s, 3H), 3.96 (s, 3H), 6.46-6.71 (m, 2H), 7.20-7.30 (m, 1H), 7.87 (br s, 1H), 12.01 (br s, 1H); ESI (m/z) 370 (M + H)⁺. |
| 52 | 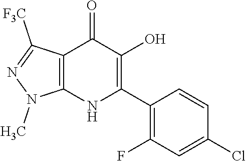 [SMILES: Cn1nc(C(F)(F)F)c2c(=O)c(O)c(-c3ccc(Cl)cc3F)[nH]c21] | A/Intermediate- 51 | 6-(4-Chloro-2-fluorophenyl)-5-hydroxy-1- methyl-3-(trifluoromethyl)-1,7-dihydro-4H- pyrazolo[3,4-b]pyridin-4-one; ¹H NMR (300 MHz, DMSO-d₆): δ 3.99 (s, 3H), 7.45-7.48 (m, 1H), 7.57-7.60 (m, 2H), 8.55 (br s, 1H), 11.24 (br s, 1H); APCI (m/z) 362 (M + H)⁺. |
| 53 | 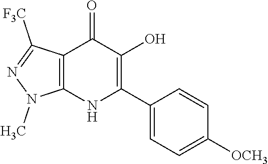 [SMILES: COc1ccc(-c2[nH]c3c(c(C(F)(F)F)nn3C)c(=O)c2O)cc1] | A/Intermediate- 52 | 5-Hydroxy-6-(4-methoxyphenyl)-1-methyl-3- (trifluoromethyl)-1,7-dihydro-4H-pyrazolo[3,4- b]pyridin-4-one; ¹H NMR (300 MHz, DMSO- d₆): δ 3.83 (s, 3H), 4.03 (s, 3H), 7.05-7.10 (m, 2H), 7.95 (br s, 2H), 10.52 (br s, 1H), 11.80 (br s, 1H); APCI (m/z) 340 (M + H)⁺. |
| 54 | 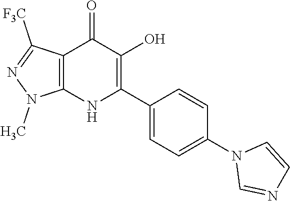 [SMILES: Cn1nc(C(F)(F)F)c2c(=O)c(O)c(-c3ccc(-n4ccnc4)cc3)[nH]c21] | A/Intermediate- 53 | 5-Hydroxy-6-[4-(1H-imidazol-1-yl)phenyl]-1- methyl-3-(trifluoromethyl)-1,7-dihydro-4H- pyrazolo[3,4-b]pyridin-4-one; ¹H NMR (300 MHz, DMSO-d₆): δ 4.05 (s, 3H), 7.16 (s, 1H), 7.78-7.87 (m, 3H), 7.90-8.21 (m, 2H), 8.39 (s, 1H); APCI (m/z) 376 (M + H)⁺. |
| 55 | 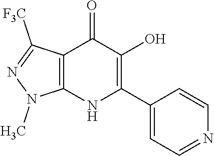 [SMILES: Cn1nc(C(F)(F)F)c2c(=O)c(O)c(-c3ccncc3)[nH]c21] | A/Intermediate- 54 | 5-Hydroxy-1-methyl-6-(pyridin-4-yl)-3- (trifluoromethyl)-1,7-dihydro-4H-pyrazolo[3,4- b]pyridin-4-one; ¹H NMR (300 MHz, DMSO- d₆): δ 4.04 (s, 3H), 7.85-7.94 (m, 2H), 8.67-8.79 (m, 2H); APCI (m/z) 309 (M − H)⁻. |
| 56 | 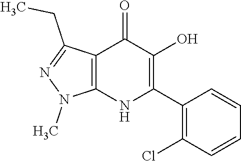 [SMILES: CCc1nn(C)c2[nH]c(-c3ccccc3Cl)c(O)c(=O)c12] | A/Intermediate- 55 | 6-(2-Chlorophenyl)-3-ethyl-5-hydroxy-1- methyl-1,7-dihydro-4H-pyrazolo[3,4-b]pyridin- 4-one; ¹H NMR (300 MHz, DMSO-d₆): δ 1.27 (t, J = 7.5 Hz, 3H), 2.87 (q, J = 7.5 Hz, 2H), 3.78 (s, 3H), 7.45-7.58 (m, 3H), 7.60-7.67 (m, 1H), 7.83-7.92 (m, 1H), 11.60 (br s, 1H); APCI (m/z) 304 (M + H)⁺. |
| 57 | 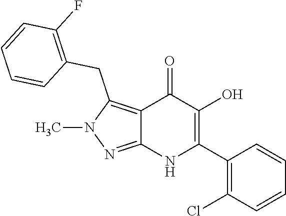 [SMILES: Cn1nc2[nH]c(-c3ccccc3Cl)c(O)c(=O)c2c1Cc1ccccc1F] | A/Intermediate- 56 | 6-(2-Chlorophenyl)-3-(2-fluorobenzyl)-5- hydroxy-2-methyl-2,7-dihydro-4H- pyrazolo[3,4-b]pyridin-4-one; ¹H NMR (300 MHz, DMSO-d₆): δ 3.83 (s, 3H), 4.56 (s, 2H), 7.10-7.18 (m, 1H), 7.21-7.30 (m, 3H), 7.45-7.49 (m, 3H), 7.59 (d, J = 7.8 Hz, 1H), 7.69 (s, 1H), 11.38 (br s, 1H); APCI (m/z) 384 (M + H)⁺. |
| 58 | 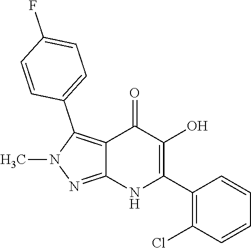 [SMILES: Cn1nc2[nH]c(-c3ccccc3Cl)c(O)c(=O)c2c1-c1ccc(F)cc1] | A/Intermediate- 57 | 6-(2-Chlorophenyl)-3-(4-fluorophenyl)-5- hydroxy-2-methyl-2,7-dihydro-4H- pyrazolo[3,4-b]pyridin-4-one; ¹H NMR (300 MHz, DMSO-d₆): δ 3.89 (s, 3H), 7.38 (t, J = 8.7 Hz, 2H), 7.45-7.65 (m, 5H), 7.76 (t, J = 5.4 Hz, 2H), 11.50 (br s, 1H); APCI (m/z) 370 (M + H)⁺. |
| 59 | 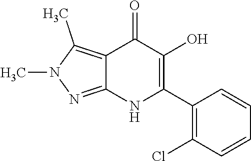 [SMILES: Cc1c2c(=O)c(O)c(-c3ccccc3Cl)[nH]c2nn1C] | A/Intermediate- 58 | 6-(2-Chlorophenyl)-5-hydroxy-2,3-dimethyl- 2,7-dihydro-4H-pyrazolo[3,4-b]pyridin-4-one; ¹H NMR (300 MHz, DMSO-d₆): δ 2.67 (s, 3H), 3.82 (s, 3H), 7.39-7.62 (m, 5H), 11.20 (br s, 1H); APCI (m/z) 290 (M + H)⁺. |
| 60 | 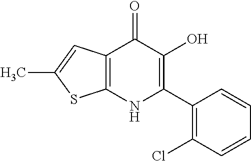 [SMILES: Cc1cc2c(=O)c(O)c(-c3ccccc3Cl)[nH]c2s1] | A/Intermediate- 60 | 6-(2-Chlorophenyl)-5-hydroxy-2- methylthieno[2,3-b]pyridin-4(7H)-one; ¹H NMR (300 MHz, DMSO-d₆): δ 2.56 (s, 3H), 7.11-7.20 (m, 1H), 7.35-7.45 (m, 3H), 8.62 (s, 1H), 10.63 (br s, 1H), 12.45 (br s, 1H); ESI (m/z) 292 (M + H)⁺. |
| 61 | 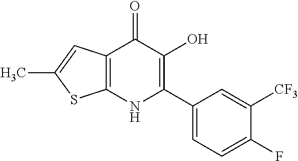 [SMILES: Cc1cc2c(=O)c(O)c(-c3ccc(F)c(C(F)(F)F)c3)[nH]c2s1] | A/Intermediate- 61 | 6-[4-Fluoro-3-(trifluoromethyl)phenyl]-5- hydroxy-2-methylthieno[2,3-b]pyridin-4(7H)- one; ¹H NMR (300 MHz, DMSO-d₆): δ 2.52 (s, 3H), 7.17 (s, 1H), 7.58 (t, J = 6.6 Hz, 1H), 8.32- 8.45 (m, 2H), 9.10 (br s, 1H), 10.86 (br s, 1H); APCI-MS (m/z) 344 (M + H)⁺. |
| 62 | 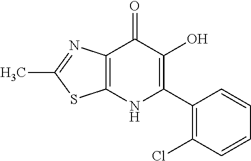 [SMILES: Cc1nc2c(=O)c(O)c(-c3ccccc3Cl)[nH]c2s1] | A/Intermediate- 66 | 5-(2-Chlorophenyl)-6-hydroxy-2- methyl[1,3]thiazolo[5,4-b]pyridin-7(4H)-one; ¹H NMR (300 MHz, DMSO-d₆): δ 2.78 (s, 3H), 7.40-7.49 (m, 3H), 7.50-7.52 (m, 1H), 9.03 (br s, 1H); APCI (m/z) 293 (M + H)⁺. |
| 63 | 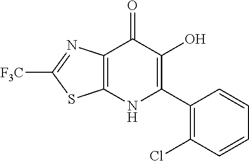 [SMILES: O=c1c(O)c(-c2ccccc2Cl)[nH]c2sc(C(F)(F)F)nc12] | A/Intermediate- 67 | 5-(2-Chlorophenyl)-6-hydroxy-2- trifluoromethyl[1,3]thiazolo[5,4-b]pyridin- 7(4H)-one; ¹H NMR (300 MHz, DMSO-d₆): δ 7.40-7.62 (m, 4H), 9.54 (br s, 1H), 12.40 (br s, 1H); APCI (m/z) 347 (M + H)⁺. |
| 64 | 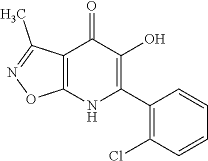 [SMILES: Cc1noc2[nH]c(-c3ccccc3Cl)c(O)c(=O)c12] | A/Intermediate- 68 | 6-(2-Chlorophenyl)-5-hydroxy-3- methyl[1,2]oxazolo[5,4-b]pyridin-4(7H)-one; ¹H NMR (300 MHz, DMSO-d₆): δ 2.54 (s, 3H), 7.42-7.56 (m, 2H), 7.55-7.58 (m, 1H); APCI (m/z) 275 (M − H)⁻. |
| 65 | 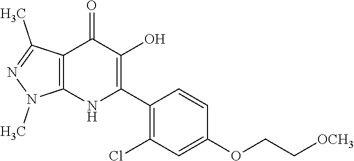 [SMILES: COCCOc1ccc(-c2[nH]c3c(c(C)nn3C)c(=O)c2O)c(Cl)c1] | A/Intermediate- 69 | 6-(2-Chloro-4-(2-methoxyethoxy)phenyl)-5- hydroxy-1,3-dimethyl-1H-pyrazolo[3,4- b]pyridin-4(7H)-one; ¹H NMR (300 MHz, DMSO-d₆): δ 2.46 (s, 3H), 3.31 (s, 3H), 3.68 (s, 2H), 3.77 (s, 3H), 4.19 (s, 2H), 7.04 (d, J = 7.4 Hz, 1H), 7.22 (s, 1H), 7.36-7.42 (m, 1H), 7.77- 7.83 (m, 1H), 11.53 (s, 1H); ESI (m/z) 364 (M + H)⁺. |
| 66 | 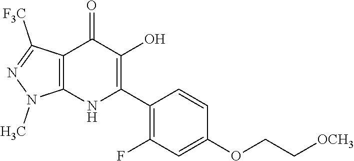 [SMILES: COCCOc1ccc(-c2[nH]c3c(c(C(F)(F)F)nn3C)c(=O)c2O)c(F)c1] | A/Intermediate- 70 | 6-(2-Fluoro-4-(2-methoxyethoxy)phenyl)-5- hydroxy-1-methyl-3-(trifluoromethyl)-1H- pyrazolo[3,4-b]pyridin-4(7H)-one; ¹H NMR (300 MHz, DMSO-d₆): δ 3.67 (s, 3H), 3.92-3.97 (m, 4H), 4.17 (s, 3H), 6.88-6.93 (m, 2H), 7.44 (t, J = 8.4 Hz, 1H), 8.26 (br s, 1H), 12.03 (br s, 1H); ESI (m/z) 400 (M − H)⁻. |
| 67 | 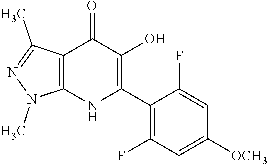 [SMILES: COc1cc(F)c(-c2[nH]c3c(c(C)nn3C)c(=O)c2O)c(F)c1] | A/Intermediate- 71 | 6-(2,6-Difluoro-4-methoxyphenyl)-5-hydroxy- 1,3-dimethyl-1H-pyrazolo[3,4-b]pyridin-4(7H)- one; ¹H NMR (300 MHz, DMSO-d₆): δ 2.45 (s, 3H), 3.76 (s, 3H), 3.85 (s, 3H), 6.90 (s, 1H), 6.94 (s, 1H), 8.12 (br s, 1H), 11.64 (s, 1H); ESI (m/z) 322 (M + H)⁺. |
| 68 | 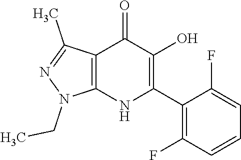 [SMILES: CCn1nc(C)c2c(=O)c(O)c(-c3c(F)cccc3F)[nH]c21] | A/Intermediate- 72 | 6-(2,6-Difluorophenyl)-1-ethyl-5-hydroxy-3- methyl-1H-pyrazolo[3,4-b]pyridin-4(7H)-one; ¹H NMR (300 MHz, DMSO-d₆): δ 1.24-1.32 (m, 3H), 3.99-4.18 (m, 2H), 7.21-7.32 (m, 2H), 7.58- 7.64 (m, 1H), 8.19-8.25 (m, 1H), 11.66 (s, 1H); ESI (m/z) 304 (M − H)⁻. |
| 69 | 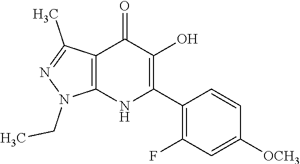 [SMILES: CCn1nc(C)c2c(=O)c(O)c(-c3ccc(OC)cc3F)[nH]c21] | A/Intermediate- 73 | 1-Ethyl-6-(2-fluoro-4-methoxyphenyl)-5- hydroxy-3-methyl-1H-pyrazolo[3,4-b]pyridin- 4(7H)-one; ¹H NMR (300 MHz, DMSO-d₆): δ 1.28 (t, J = 6.9 Hz, 3H), 2.46 (s, 3H), 3.83 (s, 3H), 4.18 (q, J = 6.9 Hz, 2H), 6.90-7.04 (m, 2H), 7.46 (t, J = 8.4 Hz, 1H), 7.85 (s, 1H), 11.47 (s, 1H); ESI (m/z) 318 (M + H)⁺. |
| 70 | 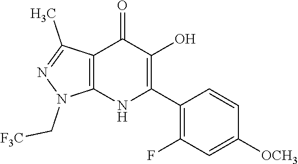 [SMILES: COc1ccc(-c2[nH]c3c(c(C)nn3CC(F)(F)F)c(=O)c2O)c(F)c1] | A/Intermediate- 74 | 6-(2-Fluoro-4-methoxyphenyl)-5-hydroxy-3- methyl-1-(2,2,2-trifluoroethyl)-1H- pyrazolo[3,4-b]pyridin-4(7H)-one; ¹H NMR (300 MHz, DMSO-d₆): δ 2.50 (s, 3H), 3.83 (s, 3H), 5.09-5.14 (m, 2H), 6.91-7.04 (m, 2H), 7.44 (t, J = 8.4 Hz, 1H), 8.11 (s, 1H), 11.75 (s, 1H); ESI (m/z) 372 (M + H)⁺. |
| 71 | 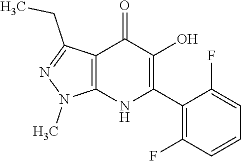 [SMILES: CCc1nn(C)c2[nH]c(-c3c(F)cccc3F)c(O)c(=O)c12] | A/Intermediate- 75 | 6-(2,6-Difluorophenyl)-3-ethyl-5-hydroxy-1- methyl-1H-pyrazolo[3,4-b]pyridin-4(7H)-one; ¹H NMR (300 MHz, DMSO-d₆): δ 1.27 (t, J = 7.8 Hz, 3H), 2.87 (q, J = 6.6 Hz, 2H), 3.78 (s, 3H), 7.20-7.35 (m, 2H), 7.50-7.68 (m, 1H), 8.25 (br s, 1H), 11.67 (br s, 1H); ESI (m/z) 306 (M + H)⁺. |
| 72 | 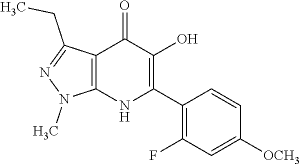 [SMILES: CCc1nn(C)c2[nH]c(-c3ccc(OC)cc3F)c(O)c(=O)c12] | A/Intermediate- 76 | 3-Ethyl-6-(2-fluoro-4-methoxyphenyl)-5- hydroxy-1-methyl-1H-pyrazolo[3,4-b]pyridin- 4(7H)-one; ¹H NMR (300 MHz, DMSO-d₆): δ 1.26 (t, J = 7.5 Hz, 3H), 2.85 (q, J = 6.9 Hz, 2H), 3.79 (s, 3H), 3.83 (s, 3H), 6.90-7.03 (m, 2H), 7.45 (t, J = 9.0 Hz, 1H), 7.86 (s, 1H), 11.51 (s, 1H); ESI (m/z) 318 (M + H)⁺. |
| 73 | 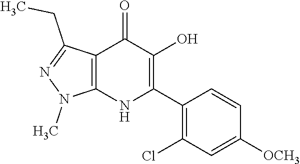 [SMILES: CCc1nn(C)c2[nH]c(-c3ccc(OC)cc3Cl)c(O)c(=O)c12] | A/Intermediate- 77 | 6-(2-Chloro-4-methoxyphenyl)-3-ethyl-5- hydroxy-1-methyl-1H-pyrazolo[3,4-b]pyridin- 4(7H)-one; ¹H NMR (300 MHz, DMSO-d₆): δ 1.26 (t, J = 7.2 Hz, 3H), 2.85 (q, J = 7.2 Hz, 2H), 3.77 (s, 3H), 3.84 (s, 3H), 7.05 (d, J = 7.5 Hz, 1H), 7.20 (s, 1H), 7.43 (d, J = 7.5 Hz, 1H), 7.79 (s, 1H), 11.54 (s, 1H); ESI (m/z) 332 (M − H)⁻. |
| 74 | 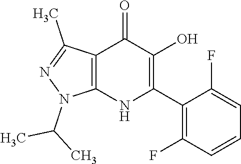 [SMILES: Cc1nn(C(C)C)c2[nH]c(-c3c(F)cccc3F)c(O)c(=O)c12] | A/Intermediate- 78 | 6-(2,6-Difluorophenyl)-5-hydroxy-1-isopropyl- 3-methyl-1H-pyrazolo[3,4-b]pyridin-4(7H)- one; ¹H NMR (300 MHz, DMSO-d₆): δ 1.35 (d, J = 6.3 Hz, 6H), 2.48 (s, 3H), 4.62-4.69 (m, 1H), 7.21-7.25 (m, 3H), 7.57-7.61 (m, 1H), 8.20 (br s, 1H), 11.59 (br s, 1H); ESI (m/z) 320 (M + H)⁺. |
| 75. | 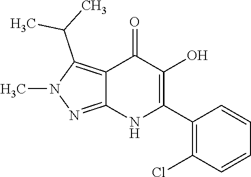 [SMILES: CC(C)c1c2c(=O)c(O)c(-c3ccccc3Cl)[nH]c2nn1C] | A/Intermediate- 79 | 6-(2-Chlorophenyl)-5-hydroxy-3-isopropyl-2- methyl-2H-pyrazolo[3,4-b]pyridin-4(7H)-one; ¹H NMR (300 MHz, DMSO-d₆): δ 1.32 (d, J = 6.0 Hz, 6H), 3.44-3.552 (m, 1H), 3.70 (s, 3H), 6.32 (s, 1H), 7.30-7.52 (m, 4H), 8.89 (s, 1H); APCI (m/z) 318 (M + H)⁺. |
| 76 | 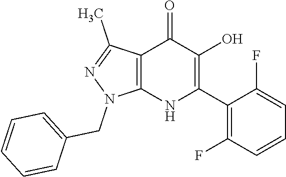 [SMILES: Cc1nn(Cc2ccccc2)c2[nH]c(-c3c(F)cccc3F)c(O)c(=O)c12] | A/Intermediate- 80 | 1-Benzyl-6-(2,6-difluorophenyl)-5-hydroxy-3- methyl-1H-pyrazolo[3,4-b]pyridin-4(7H)-one; ¹H NMR (300 MHz, DMSO-d₆): δ 5.38 (s, 2H), 7.10-7.28 (m, 7H), 7.58-7.64 (m, 1H), 8.30 (s, 1H), 11.84 (s, 1H); ESI (m/z) 368 (M + H)⁺. |
| 77 | 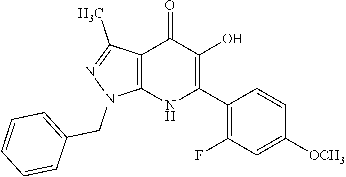 [SMILES: COc1ccc(-c2[nH]c3c(c(C)nn3Cc3ccccc3)c(=O)c2O)c(F)c1] | A/Intermediate- 81 | 1-Benzyl-6-(2-fluoro-4-methoxyphenyl)-5- hydroxy-3-methyl-1H-pyrazolo[3,4-b]pyridin- 4(7H)-one; ¹H NMR (300 MHz, DMSO-d₆): δ 2.48 (s, 3H), 3.83 (s, 3H), 5.41 (s, 2H), 6.89-6.94 (m, 3H), 7.16-7.21 (m, 2H), 7.28-7.34 (m, 3H), 7.42-7.47 (m, 1H), 7.96 (br s, 1H), 11.45 (br s, 1H); ESI (m/z) 380 (M + H)⁺. |
| 78 | 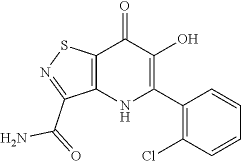 [SMILES: NC(=O)c1nsc2c(=O)c(O)c(-c3ccccc3Cl)[nH]c12] | A/Intermediate- 82 | 5-(2-Chlorophenyl)-6-hydroxy-7-oxo-4,7- dihydroisothiazolo[4,5-b]pyridine-3- carboxamide; ¹H NMR (300 MHz, DMSO-d₆): δ 7.47-7.61 (m, 4H), 8.05 (s, 1H), 8.46-8.50 (m, 1H), 9.17 (br s, 1H), 11.65 (br s, 1H); ESI (m/z) 320 (M − H)⁻. |
| 79 | 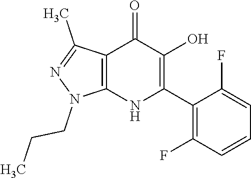 [SMILES: CCCn1nc(C)c2c(=O)c(O)c(-c3c(F)cccc3F)[nH]c21] | A/Intermediate- 83 | 6-(2,6-Difluorophenyl)-5-hydroxy-3-methyl-1- propyl-1H-pyrazolo[3,4-b]pyridin-4(7H)-one; ¹H NMR (300 MHz, DMSO-d₆): δ 0.81 (t, J = 7.5 Hz, 3H), 1.73 (q, J = 6.9 Hz, 2H), 2.47 (s, 3H), 4.07 (t, J = 6.3 Hz, 2H), 7.17-7.31 (m, 2H), 7.59-7.68 (m, 1H), 8.21 (s, 1H), 11.62 (s, 1H); ESI (m/z) 318 (M − H)⁻. |
| 80 | 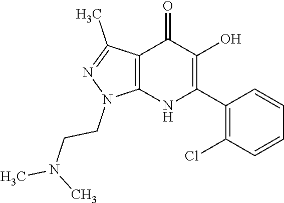 [SMILES: Cc1nn(CCN(C)C)c2[nH]c(-c3ccccc3Cl)c(O)c(=O)c12] | A/Intermediate- 84 | 6-(2-Chlorophenyl)-1-(2- (dimethylamino)ethyl)-5-hydroxy-3-methyl- 1H-pyrazolo[3,4-b]pyridin-4(7H)-one; ¹H NMR (300 MHz, DMSO-d₆): δ 2.18 (s, 3H), 2.49 (s, 6H), 2.69 (t, J = 6.2 Hz, 2H), 4.25 (t, J = 6.2 Hz, 2H), 7.42-7.60 (m, 4H); ESI (m/z) 347 (M + H)⁺. |
| 81 | 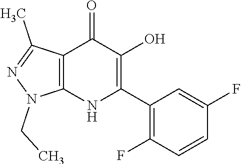 [SMILES: CCn1nc(C)c2c(=O)c(O)c(-c3cc(F)ccc3F)[nH]c21] | A/Intermediate- 85 | 6-(2,5-Difluorophenyl)-1-ethyl-5-hydroxy-3- methyl-1H-pyrazolo[3,4-b]pyridin-4(7H)-one; ¹H NMR (300 MHz, DMSO-d₆): δ 1.29 (t, J = 6.6 Hz, 3H), 3.33 (s, 3H), 4.17 (q, J = 7.5 Hz, 2H), 7.39-7.45 (m, 3H), 8.14 (br s, 1H), 11.55 (br s, 1H); APCI (m/z) 306 (M + H)⁺. |
| 82 | 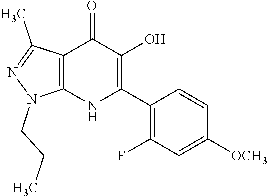 [SMILES: CCCn1nc(C)c2c(=O)c(O)c(-c3ccc(OC)cc3F)[nH]c21] | A/Intermediate- 86 | 6-(2-Fluoro-4-methoxyphenyl)-5-hydroxy-3- methyl-1-propyl-1H-pyrazolo[3,4-b]pyridin- 4(7H)-one; ¹H NMR (300 MHz, DMSO-d₆): δ 0.82 (t, J = 7.5 Hz, 3H), 1.68-1.80 (m, 2H), 2.47 (s, 3H), 3.83 (s, 3H), 4.05-4.18 (m, 2H), 6.85- 7.05 (m, 2H), 7.46 (t, J = 12 Hz, 1H), 7.84 (s, 1H), 11.44 (br s, 1H); ESI (m/z) 332 (M + H)⁺. |
| 83 | 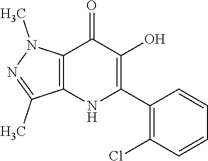 [SMILES: Cc1nn(C)c2c(=O)c(O)c(-c3ccccc3Cl)[nH]c12] | A/Intermediate- 87 | 5-(2-Chlorophenyl)-6-hydroxy-1,3-dimethyl- 1H-pyrazolo[4,3-b]pyridin-7(4H)-one; ¹H NMR (300 MHz, DMSO-d₆): δ 2.35 (s, 3H), 4.19 (s, 3H), 7.46-7.57 (m, 3H), 7.60-7.64 (m, 1H), 8.08 (s, 1H), 11.75 (s, 1H); ESI (m/z) 290 (M + H)⁺. |
| 84 | 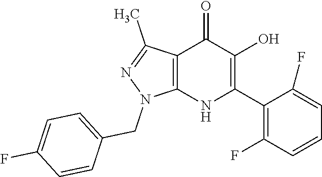 [SMILES: Cc1nn(Cc2ccc(F)cc2)c2[nH]c(-c3c(F)cccc3F)c(O)c(=O)c12] | A/Intermediate- 88 | 6-(2,6-Difluorophenyl)-1-(4-fluorobenzyl)-5- hydroxy-3-methyl-1H-pyrazolo[3,4-b]pyridin- 4(7H)-one; ¹H NMR (300 MHz, DMSO-d₆): δ 2.50 (s, 3H), 5.37 (s, 2H), 7.05-7.30 (m, 6H), 7.59 (br s, 1H), 8.31 (s, 1H), 9.70 (s, 1H); APCI (m/z) 386 (M + H)⁺. |
| 85 | 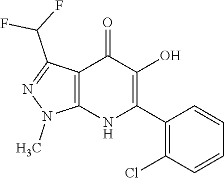 [SMILES: Cn1nc(C(F)F)c2c(=O)c(O)c(-c3ccccc3Cl)[nH]c21] | B/Intermediate- 89 | 6-(2-Chlorophenyl)-3-(difluoromethyl)-5- hydroxy-1-methyl-1H-pyrazolo[3,4-b]pyridin- 4(7H)-one; ¹H NMR (300 MHz, DMSO-d₆): δ 3.94 (s, 3H), 7.21 (t, J = 5.3 Hz, 1H), 7.40-7.65 (m, 4H); APCI (m/z) 326 (M + H)⁺. |
| 86 | 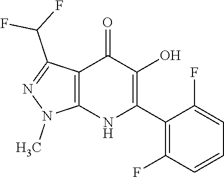 [SMILES: Cn1nc(C(F)F)c2c(=O)c(O)c(-c3c(F)cccc3F)[nH]c21] | B/Intermediate- 90 | 3-(Difluoromethyl)-6-(2,6-difluorophenyl)-5- hydroxy-1-methyl-1H-pyrazolo[3,4-b]pyridin- 4(7H)-one; ¹H NMR (300 MHz, DMSO-d₆): δ 3.95 (s, 3H), 7.24 (t, J = 54 Hz, 1H), 7.15-7.40 (m, 2H), 7.45-7.68 (m, 1H), 8.59 (br s, 1H), 12.18 (s, 1H); APCI (m/z) 326 (M − H)⁻. |
| 87 | 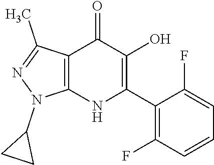 [SMILES: Cc1nn(C2CC2)c2[nH]c(-c3c(F)cccc3F)c(O)c(=O)c12] | B/Intermediate- 92 | 1-Cyclopropyl-6-(2,6-difluorophenyl)-5- hydroxy-3-methyl-1H-pyrazolo[3,4-b]pyridin- 4(7H)-one; ¹H NMR (300 MHz, DMSO-d₆): δ 0.97-1.05 (m, 4H), 2.44 (s, 3H), 3.44-3.55 (m, 1H), 7.15-7.35 (m, 2H), 7.60-7.65 (m, 1H), 8.23 (s, 1H), 11.76 (s, 1H); ESI (m/z) 318 (M + H)⁺. |
| 88 | 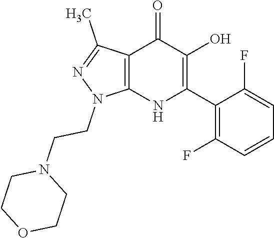 [SMILES: Cc1nn(CCN2CCOCC2)c2[nH]c(-c3c(F)cccc3F)c(O)c(=O)c12] | B/Intermediate- 93 | 6-(2,6-Difluorophenyl)-5-hydroxy-3-methyl-1- (2-morpholinoethyl)-1H-pyrazolo[3,4- b]pyridin-4(7H)-one; ¹H NMR (300 MHz, DMSO-d₆): δ 2.37-2.40 (m, 7H), 2.68 (t, J = 7.5 Hz, 2H), 3.36-3.45 (m, 4H), 4.24-4.27 (m, 2H), 7.25-7.29 (m, 3H), 7.61-7.64 (m, 1H), 8.22-8.26 (m, 1H); APCI (m/z) 389 (M − H)⁻. |
| 89 | 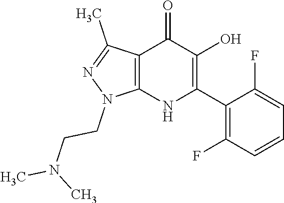 [SMILES: Cc1nn(CCN(C)C)c2[nH]c(-c3c(F)cccc3F)c(O)c(=O)c12] | B/Intermediate- 94 | 6-(2,6-Difluorophenyl)-1-(2- (dimethylamino)ethyl)-5-hydroxy-3-methyl- 1H-pyrazolo[3,4-b]pyridin-4(7H)-one; ¹H NMR (300 MHz, DMSO-d₆): δ 2.16 (s, 6H), 2.47 (s, 3H), 2.68 (t, J = 6.0 Hz, 2H), 4.22 (t, J = 6.3 Hz, 2H), 7.23 (t, J = 8.4 Hz, 2H), 7.51-7.59 (m, 1H); ESI (m/z) 349 (M + H)⁺. |
| 90 | 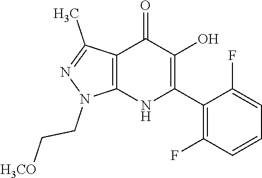 [SMILES: COCCn1nc(C)c2c(=O)c(O)c(-c3c(F)cccc3F)[nH]c21] | B/Intermediate- 95 | 6-(2,6-Difluorophenyl)-5-hydroxy-1-(2- methoxyethyl)-3-methyl-1H-pyrazolo[3,4- b]pyridin-4(7H)-one; ¹H NMR (300 MHz, DMSO-d₆): δ 2.50 (s, 3H), 3.19 (s, 3H), 3.64- 3.67 (m, 2H), 4.27-4.31 (m, 2H), 7.25-7.29 (m, 2H), 7.60-7.63 (m, 1H), 8.23 (s, 1H), 11.69 (s, 1H); APCI (m/z) 336 (M + H)⁺. |
| 91 | 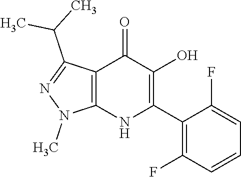 [SMILES: CC(C)c1nn(C)c2[nH]c(-c3c(F)cccc3F)c(O)c(=O)c12] | B/Intermediate- 96 | 6-(2,6-Difluorophenyl)-5-hydroxy-3-isopropyl- 1-methyl-1H-pyrazolo[3,4-b]pyridin-4(7H)one; ¹H NMR (300 MHz, DMSO-d₆): δ 1.33 (d, J = 6.3 Hz, 6H), 3.33-3.36 (m, 1H), 3.78 (s, 3H), 7.20-7.35 (m, 2H), 7.60-7.65 (m, 1H), 8.19 (br s, 1H), 11.71 (s, 1H). |
| 92 | 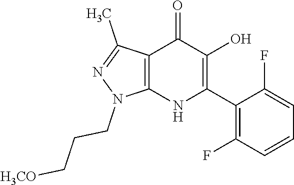 [SMILES: COCCCn1nc(C)c2c(=O)c(O)c(-c3c(F)cccc3F)[nH]c21] | B/Intermediate- 97 | 6-(2,6-Difluorophenyl)-5-hydroxy-1-(3- methoxypropyl)-3-methyl-1H-pyrazolo[3,4- b]pyridin-4(7H)-one; ¹H NMR (300 MHz, DMSO-d₆): δ 1.95 (t, J = 6.3 Hz, 2H), 2.47 (s, 3H), 3.16 (s, 3H), 3.27 (t, J = 5.7 Hz, 2H), 4.17 (t, J = 6.9 Hz, 2H), 7.26 (t, J = 7.8 Hz, 2H), 7.48- 7.65 (m, 1H), 8.31 (s, 1H), 11.68 (s, 1H); APCI (m/z) 350 (M + H)⁺. |
| 93 | 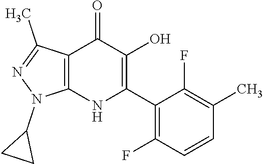 [SMILES: Cc1ccc(F)c(-c2[nH]c3c(c(C)nn3C3CC3)c(=O)c2O)c1F] | B/Intermediate- 98 | 1-Cyclopropyl-6-(2,6-difluoro-3-methylphenyl)- 5-hydroxy-3-methyl-1H-pyrazolo[3,4-b]pyridin- 4(7H)-one; ¹H NMR (300 MHz, DMSO-d₆): δ 0.96-1.05 (m, 4H), 2.28 (s, 3H), 2.44 (s, 3H), 3.46-3.51 (m, 1H), 7.16 (t, J = 9.3 Hz, 1H), 7.46-7.53 (m, 1H), 8.19 (br s, 1H), 11.75 (s, 1H); APCI (m/z) 332 (M + H)⁺. |
| 94 | 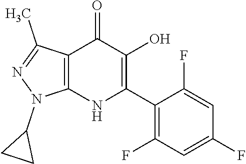 [SMILES: Cc1nn(C2CC2)c2[nH]c(-c3c(F)cc(F)cc3F)c(O)c(=O)c12] | B/Intermediate- 99 | 1-Cyclopropyl-5-hydroxy-3-methyl-6-(2,4,6- trifluorophenyl)-1H-pyrazolo[3,4-b]pyridin- 4(7H)-one; ¹H NMR (300 MHz, DMSO-d₆): δ 0.98-1.15 (m, 4H), 2.44 (s, 3H), 3.40-3.52 (m, 1H), 7.36-7.45 (m, 2H), 8.33 (s, 1H), 11.70 (s, 1H); APCI (m/z) 336 (M + H)⁺. |
| 95 | 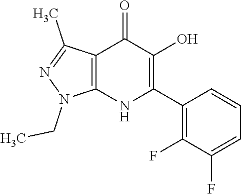 [SMILES: CCn1nc(C)c2c(=O)c(O)c(-c3cccc(F)c3F)[nH]c21] | B/Intermediate- 100 | 6-(2,3-Difluorophenyl)-1-ethyl-5-hydroxy-3- methyl-1H-pyrazolo[3,4-b]pyridin-4(7H)-one; ¹H NMR (300 MHz, DMSO-d₆): δ 1.29 (t, J = 6.9 Hz, 3H), 2.48 (s, 3H), 4.20 (q, J = 6.9 Hz, 2H), 7.35-7.41 (m, 2H), 7.54-7.59 (m, 1H), 8.17 (s, 1H), 11.57 (s, 1H); APCI (m/z) 306 (M + H)⁺. |
| 96 | 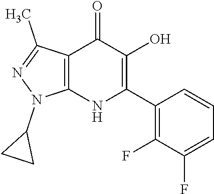 [SMILES: Cc1nn(C2CC2)c2[nH]c(-c3cccc(F)c3F)c(O)c(=O)c12] | B/Intermediate- 101 | 1-Cyclopropyl-6-(2,3-difluorophenyl)-5- hydroxy-3-methyl-1H-pyrazolo[3,4-b]pyridin- 4(7H)-one; ¹H NMR (300 MHz, DMSO-d₆): δ 0.98-1.15 (m, 4H), 2.44 (s. 3H), 3.45-3.56 (m, 1H), 7.30-7.42 (m, 2H), 7.55-7.62 (m, 1H), 8.19 (s, 1H), 11.67 (s, 1H); ESI-MS (m/z) 318 (M + H)⁺. |
| 97 | 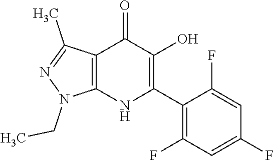 [SMILES: CCn1nc(C)c2c(=O)c(O)c(-c3c(F)cc(F)cc3F)[nH]c21] | B/Intermediate- 102 | 1-Ethyl-5-hydroxy-3-methyl-6-(2,4,6- trifluorophenyl)-1H-pyrazolo[3,4-b]pyridin- 4(7H)-one; ¹H NMR (300 MHz, DMSO-d₆): δ 1.29 (t, J = 6.9 Hz, 3H), 2.47 (s, 3H), 4.15 (q, J = 6.9 Hz, 2H), 7.35-7.50 (m, 2H), 8.31 (s, 1H), 11.63 (s, 1H); ESI-MS (m/z) 324 (M + H)⁺. |
| 98 | 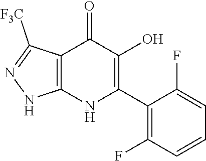 [SMILES: O=c1c(O)c(-c2c(F)cccc2F)[nH]c2[nH]nc(C(F)(F)F)c12] | B/Intermediate- 103 | 6-(2,6-Difluorophenyl)-5-hydroxy-3- (trifluoromethyl)-1H-pyrazolo[3,4-b]pyridin- 4(7H)-one; ¹H NMR (300 MHz, DMSO-d₆): δ 7.20-7.35 (m, 2H), 7.55-7.68 (m, 1H), 8.58-8.70 (m, 1H), 12.56 (br s, 1H), 14.02 (br s, 1H). |
| 99 | 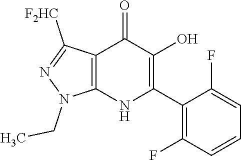 [SMILES: CCn1nc(C(F)F)c2c(=O)c(O)c(-c3c(F)cccc3F)[nH]c21] | B/Intermediate- 104 | 3-(Difluoromethyl)-6-(2,6-difluorophenyl)-1- ethyl-5-hydroxy-1H-pyrazolo[3,4-b]pyridin- 4(7H)-one; ¹H NMR (300 MHz, DMSO-d₆): δ 1.37 (t, J = 6.9 Hz, 3H), 4.35 (q, J = 6.9 Hz, 2H), 7.24 (t, J = 54 Hz, 1H), 7.15-7.40 (m, 2H), 7.48- 7.65 (m, 1H), 8.55-8.59 (m, 1H), 12.11 (br s, 1H); APCI-MS (m/z) 342 (M + H)⁺. |
| 100 | 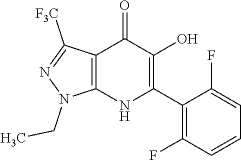 [SMILES: CCn1nc(C(F)(F)F)c2c(=O)c(O)c(-c3c(F)cccc3F)[nH]c21] | B/Intermediate- 105 | 6-(2,6-Difluorophenyl)-1-ethyl-5-hydroxy-3- (trifluoromethyl)-1H-pyrazolo[3,4-b]pyridin- 4(7H)-one; ¹H NMR (300 MHz, DMSO-d₆): δ 1.38 (t, J = 7.2 Hz, 3H), 4.39 (q, J = 7.2 Hz, 2H), 7.18-7.38 (m, 2H), 7.55-7.65 (m, 1H), 8.67 (br s, 1H), 12.30 (br s, 1H); APCI-MS (m/z) 360 (M + H)⁺. |
| 101 | 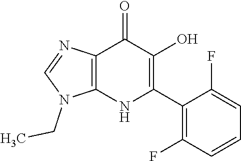 | B/Intermediate- 106 | 5-(2,6-Difluorophenyl)-3-ethyl-6-hydroxy-3H- imidazo[4,5-b]pyridin-7(4H)-one; ¹H NMR (300 MHz, DMSO-d₆): δ 1.39 (t, J = 7.2 Hz, 3H), 4.16 (q, J = 7.2 Hz, 2H), 7.15 (t, J = 7.8 Hz, 2H), 7.40-7.52 (m, 1H), 8.27 (s, 1H); APCI (m/z) 392 (M + H)⁺. |
| 102 | 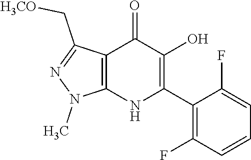 [SMILES: COCc1nn(C)c2[nH]c(-c3c(F)cccc3F)c(O)c(=O)c12] | B/Intermediate- 107 | 6-(2,6-Difluorophenyl)-5-hydroxy-3- (methoxymethyl)-1-methyl-1H-pyrazolo[3,4- b]pyridin-4(7H)-one; ¹H NMR (300 MHz, DMSO-d₆): δ 3.34 (s, 3H), 3.85 (s, 3H), 4.68 (s, 2H), 7.20-7.35 (m, 2H), 7.55-7.68 (m, 1H), 8.40 (br s, 1H), 11.86 (br s, 1H); APCI (m/z) 323 (M + H)⁺. |
| 103 | 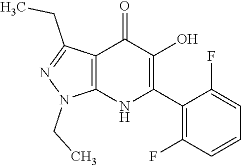 [SMILES: CCc1nn(CC)c2[nH]c(-c3c(F)cccc3F)c(O)c(=O)c12] | B/Intermediate- 108 | 6-(2,6-Difluorophenyl)-1,3-diethyl-5-hydroxy- 1,7-dihydro-4H-pyrazolo[3,4-b]pyridin-4-one; ¹H NMR (300 MHz, DMSO-d₆): δ 1.20-1.35 (m, 6H), 2.80-2.95 (m, 2H), 4.15-4.24 (m, 2H), 7.20- 7.35 (m, 2H), 7.60-7.68 (m, 1H), 8.23 (br s, 1H), 11.68 (s, 1H); APCI-MS (m/z) 320 (M + H)⁺. |
| 104 | 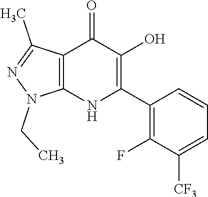 [SMILES: CCn1nc(C)c2c(=O)c(O)c(-c3cccc(C(F)(F)F)c3F)[nH]c21] | B/Intermediate- 109 | 1-Ethyl-6-[2-fluoro-3-(trifluoromethyl)phenyl]- 5-hydroxy-3-methyl-1,7-dihydro-4H- pyrazolo[3,4-b]pyridin-4-one; ¹H NMR (300 MHz, DMSO-d₆): δ 1.30 (t, J = 7.2 Hz, 3H), 2.50 (s, 3H), 4.18 (q, J = 7.2 Hz, 2H), 7.54 (t, J = 7.8 Hz, 1H), 7.82-7.95 (m, 2H); APCI-MS (m/z) 356 (M + H)⁺. |
| 105 | 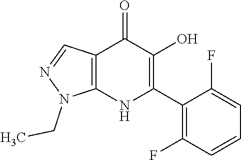 [SMILES: CCn1ncc2c(=O)c(O)c(-c3c(F)cccc3F)[nH]c21] | B/Intermediate- 110 | 6-(2,6-Difluorophenyl)-1-ethyl-5-hydroxy-1,7- dihydro-4H-pyrazolo[3,4-b]pyridin-4-one; ¹H NMR (300 MHz, DMSO-d₆): δ 1.34 (t, J = 7.2 Hz, 3H), 4.27 (q, J = 12 Hz, 2H), 7.20 (t, J = 7.8 Hz, 2H), 7.50-7.59 (m, 1H), 8.10 (s, 1H), 8.42 (s, 1H), 11.90 (br s, 1H). |
| 106 | 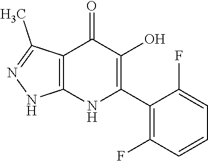 [SMILES: Cc1n[nH]c2[nH]c(-c3c(F)cccc3F)c(O)c(=O)c12] | B/Intermediate- 111 | 6-(2,6-Difluorophenyl)-5-hydroxy-3-methyl- 1,7-dihydro-4H-pyrazolo[3,4-b]pyridin-4-one; ¹H NMR (300 MHz, DMSO-d₆): δ 2.56 (s, 3H), 7.23 (t, J = 7.8 Hz, 2H), 7.58 (t, J = 7.5 Hz, 1H), 7.89 (br s, 1H), 11.35 (br s, 1H), 12.97 (br s, 1H); ESI-MS (m/z) 278 (M + H)⁺. |
| 107 | 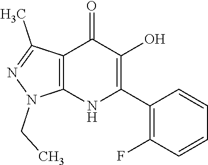 [SMILES: CCn1nc(C)c2c(=O)c(O)c(-c3ccccc3F)[nH]c21] | B/Intermediate- 112 | 1-Ethyl-6-(2-fluorophenyl)-5-hydroxy-3- methyl-1,7-dihydro-4H-pyrazolo[3,4-b]pyridin- 4-one; ¹H NMR (300 MHz, DMSO-d₆): δ 1.28 (t, J = 6.9 Hz, 3H), 2.47 (s, 3H), 4.18 (q, J = 6.9 Hz, 2H), 7.32-7.40 (m, 2H), 7.46-7.60 (m, 2H), 7.95 (s, 1H), 11.54 (s, 1H); APCI-MS (m/z) 287 (M + H)⁺. |
| 108 | 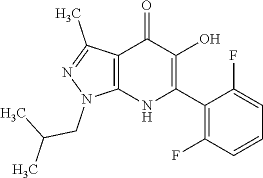 [SMILES: Cc1nn(CC(C)C)c2[nH]c(-c3c(F)cccc3F)c(O)c(=O)c12] | B/Intermediate- 113 | 6-(2,6-Difluorophenyl)-5-hydroxy-1-isobutyl-3- methyl-1H-pyrazolo[3,4-b]pyridin-4(7H)-one; ¹H NMR (300 MHz, DMSO-d₆): δ 0.82 (d, J = 6.9 Hz, 6H), 2.05-2.18 (m, 1H), 3.32 (s, 3H), 3.95 (d, J = 6.9 Hz, 2H), 7.20-7.33 (m, 2H), 7.57-7.68 (m, 1H), 8.22 (br s, 1H), 11.58 (br s, 1H); APCI-MS (m/z) 334 (M + H)⁺. |
| 109 | 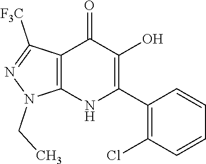 [SMILES: CCn1nc(C(F)(F)F)c2c(=O)c(O)c(-c3ccccc3Cl)[nH]c21] | B/Intermediate- 114 | 6-(2-Chlorophenyl)-1-ethyl-5-hydroxy-3- (trifluoromethyl)-1H-pyrazolo[3,4-b]pyridin- 4(7H)-one; ¹H NMR (300 MHz, DMSO-d₆): δ 1.37 (t, J = 6.9 Hz, 3H), 4.38 (q, J = 6.9 Hz, 2H), 7.40-7.70 (m, 4H), 8.31 (br s, 1H), 12.09 (br s, 1H); APCI-MS (m/z) 356 (M + H)⁺. |
| 110 | 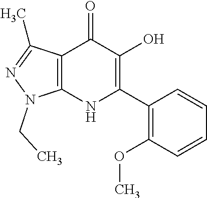 | B/Intermediate- 115 | 1-Ethyl-5-hydroxy-6-(2-methoxyphenyl)-3- methyl-1H-pyrazolo[3,4-b]pyridin-4(7H)-one; ¹H NMR (300 MHz, DMSO-d₆): δ 1.26 (t, J = 6.9 Hz, 3H), 2.46 (s, 3H), 3.76 (s, 3H), 4.16 (q, J = 7.2 Hz, 2H), 7.05 (t, J = 6.6 Hz, 1H), 7.16 (d, J = 8.7 Hz, 1H), 7.36 (d, J = 7.5 Hz, 1H), 7.46 (t, J = 7.8 Hz, 1H), 7.54 (s, 1H), 11.38 (s, 1H); APCI-MS (m/z) 300 (M + H)⁺. |
| 111 | 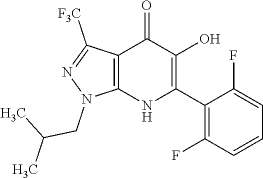 [SMILES: CC(C)Cn1nc(C(F)(F)F)c2c(=O)c(O)c(-c3c(F)cccc3F)[nH]c21] | B/Intermediate- 116 | 6-(2,6-Difluorophenyl)-5-hydroxy-1-isobutyl-3- (trifluoromethyl)-1H-pyrazolo[3,4-b]pyridin- 4(7H)-one; ¹H NMR (300 MHz, DMSO-d₆): δ 0.81 (d, J = 6.9 Hz, 6H), 2.10-2.21 (m, 1H), 4.13-4.20 (m, 2H), 7.20-7.27 (m, 2H), 7.56 (br s, 1H); APCI-MS (m/z) 388 (M + H)⁺. |
| 112 | 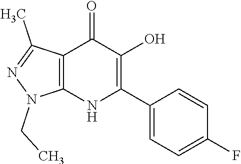 [SMILES: CCn1nc(C)c2c(=O)c(O)c(-c3ccc(F)cc3)[nH]c21] | B/Intermediate- 117 | 1-Ethyl-6-(4-fluorophenyl)-5-hydroxy-3- methyl-1,7-dihydro-4H-pyrazolo[3,4-b]pyridin- 4-one; ¹H NMR (300 MHz, DMSO-d₆): δ 1.31 (t, J = 6.9 Hz, 3H), 3.32 (s, 3H), 4.23 (q, J = 6.9 Hz, 2H), 7.28-7.42 (m, 2H), 7.70-8.05 (m, 3H), 11.29 (m, 1H); APCI-MS (m/z) 288 (M + H)⁺. |
| 113 | 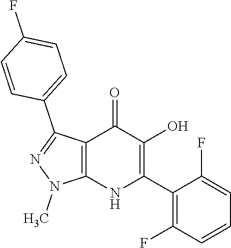 [SMILES: Cn1nc(-c2ccc(F)cc2)c2c(=O)c(O)c(-c3c(F)cccc3F)[nH]c21] | B/Intermediate- 118 | 6-(2,6-Difluorophenyl)-3-(4-fluorophenyl)-5- hydroxy-1-methyl-1H-pyrazolo[3,4-b]pyridin- 4(7H)-one; ¹H NMR (300 MHz, DMSO-d₆): δ 3.92 (s, 3H), 7.20-7.32 (m, 4H), 7.60-7.70 (m, 1H), 8.32-8.35 (m, 1H), 8.50-8.55 (m, 2H), 12.01 (s, 1H); APCI-MS (m/z) 370 (M − H)⁻. |
| 114 | 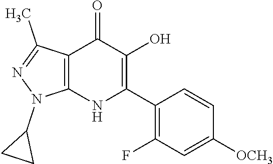 [SMILES: COc1ccc(-c2[nH]c3c(c(C)nn3C3CC3)c(=O)c2O)c(F)c1] | A/Intermediate- 119 | 1-Cyclopropyl-6-(2-fluoro-4-methoxyphenyl)- 5-hydroxy-3-methyl-1H-pyrazolo[3,4-b]pyridin- 4(7H)-one; ¹H NMR (300 MHz, DMSO-d₆): δ 0.98-1.15 (m, 4H), 2.44 (s, 3H), 3.46-3.58 (m, 1H), 3.83 (s, 3H), 6.85-7.08 (m, 2H), 7.46 (t, J = 6.7 Hz, 1H), 7.80-7.87 (m, 1H), 11.55 (br s, 1H); ESI (m/z) 330 (M + H)⁺. |
| 115 | 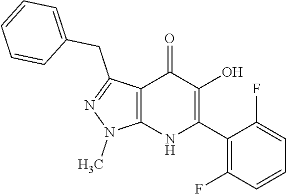 [SMILES: Cn1nc(Cc2ccccc2)c2c(=O)c(O)c(-c3c(F)cccc3F)[nH]c21] | A/Intermediate- 120 | 3-Benzyl-6-(2,6-difluorophenyl)-5-hydroxy-1- methyl-1H-pyrazolo[3,4-b]pyridin-4(7H)-one; ¹H NMR (300 MHz, DMSO-d₆): δ 3.78 (s, 3H), 4.22 (s, 2H), 7.13-7.34 (m, 5H), 7.39 (d, J = 6.6 Hz, 2H), 7.61-7.65 (m, 1H), 8.34 (s, 1H), 11.79 (s, 1H); ESI m/z) 368 (M + H)⁺. |
| 116 | 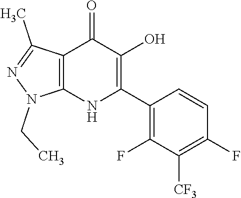 [SMILES: CCn1nc(C)c2c(=O)c(O)c(-c3ccc(F)c(C(F)(F)F)c3F)[nH]c21] | B/Intermediate- 121 | 6-(2,4-Difluoro-3-(trifluoromethyl)phenyl)-1- ethyl-5-hydroxy-3-methyl-1H-pyrazolo[3,4- b]pyridin-4(7H)-one; ¹H NMR (300 MHz, DMSO-d₆): δ 1.30 (t, J = 6.9 Hz, 3H), 2.47 (s, 3H), 4.12 (q, J = 6.9 Hz, 2H), 7.40-7.65 (m, 1H), 7.85-8.15 (m, 1H), 8.26-8.35 (m, 1H), 11.54 (br s, 1H); APCI-MS (m/z) 374 (M + H)⁺. |
| 117 | 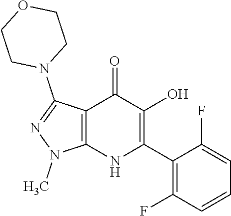 [SMILES: Cn1nc(N2CCOCC2)c2c(=O)c(O)c(-c3c(F)cccc3F)[nH]c21] | B/Intermediate- 122 | 6-(2,6-Difluorophenyl)-5-hydroxy-1-methyl-3- morpholino-1H-pyrazolo[3,4-b]pyridin-4(7H)- one; ¹H NMR (300 MHz, DMSO-d₆): δ 3.25 (s, 3H), 3.31-3.40 (m, 4H), 3.65-3.78 (m, 4H), 7.20- 7.34 (m, 2H), 7.42 (s, 1H), 7.53-7.58 (m, 1H); APCI-MS (m/z) 362 (M + H)⁺. |
| 118 | 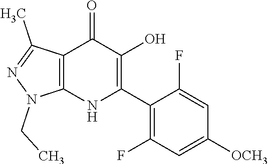 [SMILES: CCn1nc(C)c2c(=O)c(O)c(-c3c(F)cc(OC)cc3F)[nH]c21] | A/Intermediate- 123 | 6-(2,6-Difluoro-4-methoxyphenyl)-1-ethyl-5- hydroxy-3-methyl-1H-pyrazolo[3,4-b]pyridin- 4(7H)-one; ¹H NMR (300 MHz, DMSO-d₆): δ 1.25 (t, J = 6.9 Hz, 3H), 2.47 (s, 3H), 3.83 (s, 3H), 4.12 (q, J = 6.9 Hz, 2H), 6.90 (d, J = 9.6 Hz, 2H), 8.07 (s, 1H), 11.57 (s, 1H). |
| 119 | 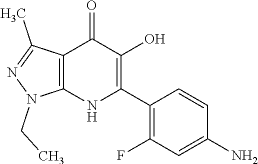 [SMILES: CCn1nc(C)c2c(=O)c(O)c(-c3ccc(N)cc3F)[nH]c21] | B/Intermediate- 124 | 6-(4-Amino-2-fluorophenyl)-1-ethyl-5- hydroxy-3-methyl-1,7-dihydro-4H- pyrazolo[3,4-b]pyridin-4-one; ¹H NMR (300 MHz, DMSO-d₆): δ 1.27 (t, J = 6.9 Hz, 3H), 2.46 (s, 3H), 4.18 (q, J = 6.9 Hz, 2H), 5.72 (s, 2H), 6.38-6.54 (m, 2H), 7.16 (t, J = 8.4 Hz, 1H), 7.61 (s, 1H), 11.33 (s, 1H); APCI (m/z) 303 (M + H)⁺. |
| 120 | 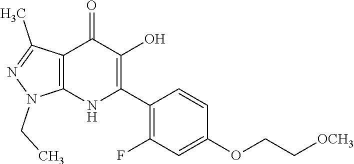 [SMILES: CCn1nc(C)c2c(=O)c(O)c(-c3ccc(OCCOC)cc3F)[nH]c21] | A/Intermediate- 127 | 1-Ethyl-6-[2-fluoro-4-(2- methoxyethoxy)phenyl]-5-hydroxy-3-methyl- 1,7-dihydro-4H-pyrazolo[3,4-b]pyridin-4-one; ¹H NMR (300 MHz, DMSO-d₆): δ 1.28 (t, J = 6.9 Hz, 3H), 2.47 (s, 3H), 3.32 (s, 3H), 3.68 (t, J = 6.3 Hz, 2H), 4.10-4.25 (m, 4H), 6.92 (d, J = 9.6 Hz, 1H), 7.01 (d, J = 9.6 Hz, 1H), 7.45 (t, J = 8.4 Hz, 1H), 7.87 (s, 1H), 11.47 (s, 1H); APCI (m/z) 362 (M + H)⁺. |
| 121 | 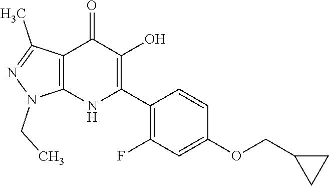 [SMILES: CCn1nc(C)c2c(=O)c(O)c(-c3ccc(OCC4CC4)cc3F)[nH]c21] | A/Intermediate- 128 | 6-[4-(Cyclopropylmethoxy)-2-fluorophenyl]-1- ethyl-5-hydroxy-3-methyl-1,7-dihydro-4H- pyrazolo[3,4-b]pyridin-4-one; ¹H NMR (300 MHz, DMSO-d₆): δ 1.27 (t, J = 6.9 Hz, 3H), 1.33-1.65 (m, 4H), 2.00-2.19 (m, 1H), 2.46 (s, 3H), 2.77 (br s, 2H), 4.18 (q, J = 6.9 Hz, 2H), 6.73 (d, J = 8.4 Hz, 1H), 7.23 (s, 1H), 7.82 (s, 1H), 11.45 (s, 1H); APCI (m/z) 358 (M + H)⁺. |
| 122 | 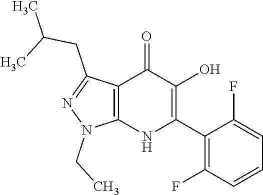 [SMILES: CCn1nc(CC(C)C)c2c(=O)c(O)c(-c3c(F)cccc3F)[nH]c21] | B/Intermediate- 129 | 6-(2,6-Difluorophenyl)-1-ethyl-5-hydroxy-3-(2- methylpropyl)-1,7-dihydro-4H-pyrazolo[3,4- b]pyridin-4-one; ¹H NMR (300 MHz, DMSO-d₆): δ 0.91 (d, J = 6.3 Hz, 6H), 1.27 (t, J = 6.9 Hz, 3H), 2.10-2.25 (m, 1H), 2.74 (d, J = 6.9 Hz, 2H), 4.17 (q, J = 7.2 Hz, 2H), 7.28 (t, J = 8.1 Hz, 2H), 7.57-7.68 (m, 1H), 8.22 (s, 1H), 11.69 (s, 1H); APCI (m/z) 348 (M + H)⁺. |
| 123 | 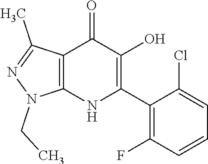 [SMILES: CCn1nc(C)c2c(=O)c(O)c(-c3c(F)cccc3Cl)[nH]c21] | B/Intermediate- 130 | 6-(2-Chloro-6-fluorophenyl)-1-ethyl-5- hydroxy-3-methyl-1,7-dihydro-4H- pyrazolo[3,4-b]pyridin-4-one; ¹H NMR (300 MHz, DMSO-d₆): δ 1.27 (t, J = 7.2 Hz, 3H), 2.47 (s, 3H), 4.14 (q, J = 7.2 Hz, 2H), 7.40 (t, J = 7.8 Hz, 1H), 7.52 (d, J = 7.8 Hz, 1H), 7.56-7.65 (m, 1H), 8.18 (s, 1H), 11.67 (s, 1H); APCI (m/z) 322 (M + H)⁺. |
| 124 | 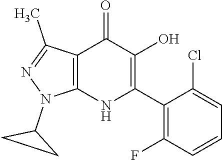 [SMILES: Cc1nn(C2CC2)c2[nH]c(-c3c(F)cccc3Cl)c(O)c(=O)c12] | A/Intermediate- 131 | 6-(2-Chloro-6-fluorophenyl)-1-cyclopropyl-5- hydroxy-3-methyl-1,7-dihydro-4H- pyrazolo[3,4-b]pyridin-4-one; ¹H NMR (300 MHz, DMSO-d₆): δ 0.95-1.10 (m, 4H), 2.42 (s, 3H), 3.41-3.48 (m, 1H), 7.35-7.60 (m, 3H), 8.16 (s, 1H), 11.73 (s, 1H); APCI (m/z) 334 (M + H)⁺. |
| 125 | 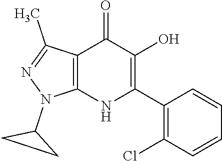 [SMILES: Cc1nn(C2CC2)c2[nH]c(-c3ccccc3Cl)c(O)c(=O)c12] | A/Intermediate- 132 | 6-(2-Chlorophenyl)-1-cyclopropyl-5-hydroxy- 3-methyl-1,7-dihydro-4H-pyrazolo[3,4- b]pyridin-4-one; ¹H NMR (300 MHz, DMSO- d₆): δ 0.95-1.15 (m, 4H), 2.44 (s, 3H), 3.45-3.60 (m, 1H), 7.40-7.65 (m, 3H), 7.57-7.64 (m, 1H), 7.89 (s, 1H), 11.69 (s, 1H); APCI (m/z) 316 (M + H)⁺. |
| 126 | 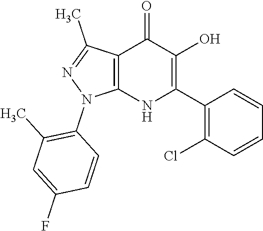 [SMILES: Cc1cc(F)ccc1-n1nc(C)c2c(=O)c(O)c(-c3ccccc3Cl)[nH]c21] | B/Intermediate- 133 | 6-(2-Chlorophenyl)-1-(4-fluoro-2- methylphenyl)-5-hydroxy-3-methyl-1,7- dihydro-4H-pyrazolo[3,4-b]pyridin-4-one; ¹H NMR (300 MHz, DMSO-d₆): δ 2.02 (s, 3H), 2.55 (s, 3H), 7.10-7.60 (m, 7H), 8.03 (s, 1H), 11.60 (s, 1H) APCI (m/z) 384 (M + H)⁺. |
| 127 | 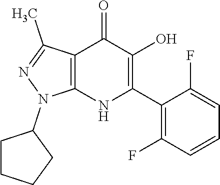 [SMILES: Cc1nn(C2CCCC2)c2[nH]c(-c3c(F)cccc3F)c(O)c(=O)c12] | B/Intermediate- 134 | 1-Cyclopentyl-6-(2,6-difluorophenyl)-5- hydroxy-3-methyl-1,7-dihydro-4H- pyrazolo[3,4-b]pyridin-4-one; ¹H NMR (300 MHz, DMSO-d₆): δ 1.50-1.70 (m, 2H), 1.80- 2.05 (m, 6H), 2.48 (s, 3H), 4.78-4.86 (m, 1H), 7.15-7.30 (m, 2H), 7.57-7.68 (m, 1H), 8.19 (s, 1H), 11.59 (s, 1H); APCI (m/z) 346 (M + H)⁺. |
| 128 | 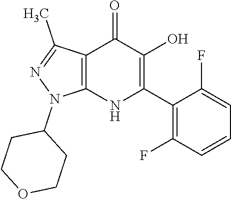 [SMILES: Cc1nn(C2CCOCC2)c2[nH]c(-c3c(F)cccc3F)c(O)c(=O)c12] | B/Intermediate- 135 | 6-(2,6-Difluorophenyl)-5-hydroxy-3-methyl-1- (tetrahydro-2H-pyran-4-yl)-1,7-dihydro-4H- pyrazolo[3,4-b]pyridin-4-one; ¹H NMR (300 MHz, DMSO-d₆): δ 1.70-1.85 (m, 2H), 1.90- 2.10 (m, 2H), 2.48 (s, 3H), 3.40 (t, J = 10.8 Hz, 2H), 3.90-4.00 (m, 2H), 4.54-4.75 (m, 1H), 7.18- 7.35 (m, 2H), 7.48-7.65 (m, 1H), 8.26 (s, 1H), 11.69 (s, 1H); APCI (m/z) 362 (M + H)⁺. |
| 129 | 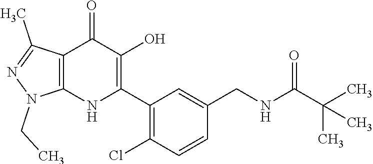 [SMILES: CCn1nc(C)c2c(=O)c(O)c(-c3cc(CNC(=O)C(C)(C)C)ccc3Cl)[nH]c21] | B/Intermediate- 136 | N-(4-Chloro-3-(1-ethyl-5-hydroxy-3-methyl-4- oxo-4,7-dihydro-1H-pyrazolo[3,4-b]pyridin-6- yl)benzyl)pivalamide; ¹H NMR (300 MHz, CDCl₃): δ 1.21 (s, 9H), 1.42 (t, J = 7.2 Hz, 3H), 2.64 (s, 3H), 4.24 (q, J = 7.2 Hz, 2H), 4.32 (s, 2H), 6.96 (br s, 1H), 7.15-7.32 (m, 2H), 7.41 (d, J = 8.4 Hz, 1H); APCI (m/z) 417 (M + H)⁺. |
| 130 | 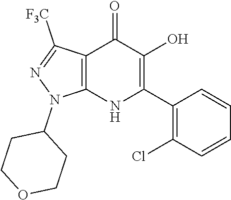 [SMILES: O=c1c(O)c(-c2ccccc2Cl)[nH]c2c1c(C(F)(F)F)nn2C1CCOCC1] | B/Intermediate- 137 | 6-(2-Chlorophenyl)-5-hydroxy-1-(tetrahydro- 2H-pyran-4-yl)-3-(trifluoromethyl)-1H- pyrazolo[3,4-b]pyridin-4(7H)-one; ¹H NMR (300 MHz, DMSO-d₆): δ 1.80-2.26 (m, 4H), 3.25-3.64 (m, 2H), 3.90-4.05 (m, 2H), 4.82-5.05 (m, 1H), 7.38-7.70 (m, 3H), 8.36 (s, 1H), 11.08 (br s, 1H), 12.14 (br s, 1H); APCI (m/z) 414 (M + H)⁺. |
| 131 | 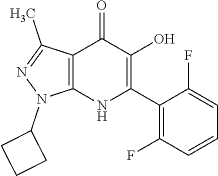 [SMILES: Cc1nn(C2CCC2)c2[nH]c(-c3c(F)cccc3F)c(O)c(=O)c12] | B/Intermediate- 138 | 1-Cyclobutyl-6-(2,6-difluorophenyl)-5- hydroxy-3-methyl-1H-pyrazolo[3,4-b]pyridin- 4(7H)-one; ¹H NMR (300 MHz, DMSO-d₆): δ 1.65-1.85 (m, 2H), 2.24-2.40 (m, 2H), 2.41-2.65 (m, 5H), 5.02 (br s, 1H), 7.10-7.38 (m, 2H), 7.50- 7.68 (m, 1H), 8.26 (s, 1H), 11.65 (br s, 1H); APCI (m/z) 332 (M + H)⁺. |
| 132 | 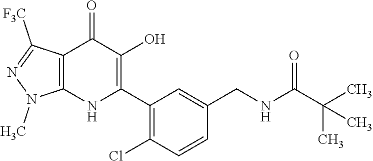 [SMILES: Cn1nc(C(F)(F)F)c2c(=O)c(O)c(-c3cc(CNC(=O)C(C)(C)C)ccc3Cl)[nH]c21] | B/Intermediate- 139 | N-(4-Chloro-3-(5-hydroxy-1-methyl-4-oxo-3- (trifluoromethyl)-4,7-dihydro-1H-pyrazolo[3,4- b]pyridin-6-yl)benzyl)pivalamide; ¹H NMR (300 MHz, DMSO-d₆): δ 1.12 (s, 9H), 3.96 (s, 3H), 4.29 (d, J = 6.0 Hz, 2H), 7.25-7.44 (m, 2H), 7.45-7.66 (m, 1H), 8.20 (s, 1H), 11.03 (br s, 1H), 12.23 (br s, 1H); APCI (m/z) 457 (M + H)⁺. |
| 133 | 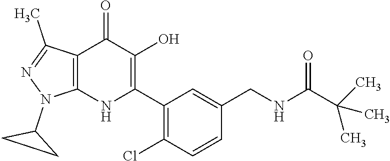 [SMILES: Cc1nn(C2CC2)c2[nH]c(-c3cc(CNC(=O)C(C)(C)C)ccc3Cl)c(O)c(=O)c12] | A/Intermediate- 140 | N-(4-Chloro-3-(1-cyclopropyl-5-hydroxy-3- methyl-4-oxo-4,7-dihydro-1H-pyrazolo[3,4- b]pyridin-6-yl)benzyl)pivalamide; ¹H NMR (300 MHz, DMSO-d₆): δ 0.90-1.09 (m, 4H), 1.12 (s, 9H), 2.44 (s, 3H), 3.50-3.54 (m, 1H), 4.28 (d, J = 5.4 Hz, 2H), 7.32-7.39 (m, 2H), 7.55 (d, J = 8.4 Hz, 1H), 7.87 (br s, 1H), 8.19 (br s, 1H), 11.72 (s, 1H); APCI (m/z) 429 (M)⁺. |
| 134 | 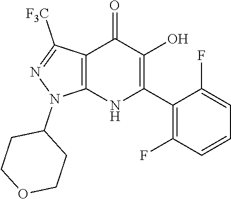 [SMILES: O=c1c(O)c(-c2c(F)cccc2F)[nH]c2c1c(C(F)(F)F)nn2C1CCOCC1] | B/Intermediate- 141 | 6-(2,6-Difluorophenyl)-5-hydroxy-1- (tetrahydro-2H-pyran-4-yl)-3-(trifluoromethyl)- 1,7-dihydro-4H-pyrazolo[3,4-b]pyridin-4-one; ¹H NMR (300 MHz, DMSO-d₆): δ 1.92-1.95 (m, 2H), 1.96-2.07 (m, 2H), 3.33-3.52 (m, 2H), 3.93- 3.97 (m, 2H), 4.90-5.05 (m, 1H), 7.15-7.35 (m, 2H), 7.48-7.65 (m, 1H), 8.70 (s, 1H), 12.28 (s, 1H); APCI (m/z) 416 (M + H)⁺. |
| 135 | 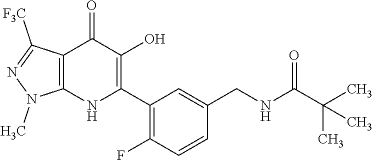 [SMILES: Cn1nc(C(F)(F)F)c2c(=O)c(O)c(-c3cc(CNC(=O)C(C)(C)C)ccc3F)[nH]c21] | B/Intermediate- 142 | N-(4-Fluoro-3-(5-hydroxy-1-methyl-4-oxo-3- (trifluoromethyl)-4,7-dihydro-1H-pyrazolo[3,4- b]pyridin-6-yl)benzyl)pivalamide; ¹H NMR (300 MHz, DMSO-d₆): δ 1.10 (s, 9H), 3.97 (s, 3H), 4.28 (d, J = 5.7 Hz, 2H), 7.15-7.45 (m, 3H), 8.10-8.20 (m, 1H), 10.98 (br s, 1H), 12.14 (br s, 1H); APCI (m/z) 441 (M + H)⁺. |
| 136 | 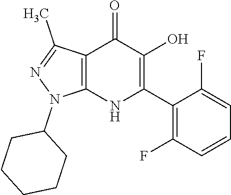 [SMILES: Cc1nn(C2CCCCC2)c2[nH]c(-c3c(F)cccc3F)c(O)c(=O)c12] | B/Intermediate- 143 | 1-Cyclohexyl-6-(2,6-difluorophenyl)-5- hydroxy-3-methyl-1,7-dihydro-4H- pyrazolo[3,4-b]pyridin-4-one; ¹H NMR (300 MHz, DMSO-d₆): δ 1.08-1.45 (m, 3H), 1.60- 1.90 (m, 7H), 2.47 (s, 3H), 4.28-4.40 (m, 1H), 7.28 (t, J = 7.8 Hz, 2H), 7.60-7.65 (m, 1H), 8.21 (s, 1H), 11.58 (s, 1H); APCI (m/z) 360 (M + H)⁺. |
| 137 | 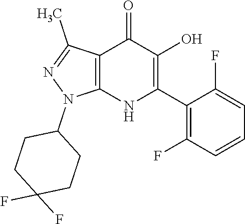 | B/Intermediate- 144 | 1-(4,4-Difluorocyclohexyl)-6-(2,6- difluorophenyl)-5-hydroxy-3-methyl-1H- pyrazolo[3,4-b]pyridin-4(7H)-one; ¹H NMR (300 MHz, DMSO-d₆): δ 1.25-1.45 (m, 2H), 1.68-1.80 (m, 2H), 1.90-2.10 (m, 4H), 3.10 (s, 3H), 4.35-4.45 (m, 1H), 7.15-7.38 (m, 2H), 7.58- 7.65 (m, 1H), 8.19 (s, 1H), 11.57 (s, 1H). APCI (m/z) 388 (M + H)⁺. |
| 138 | 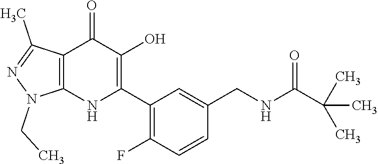 [SMILES: CCn1nc(C)c2c(=O)c(O)c(-c3cc(CNC(=O)C(C)(C)C)ccc3F)[nH]c21] | B/Intermediate- 145 | N-(3-(1-Ethyl-5-hydroxy-3-methyl-4-oxo-4,7- dihydro-1H-pyrazolo[3,4-b]pyridin-6-yl)-4- fluorobenzyl)pivalamide; ¹H NMR (300 MHz, DMSO-d₆): δ 1.12 (s, 9H), 1.27 (t, J = 6.9 Hz, 3H), 2.47 (s, 3H), 4.17 (q, J = 6.9 Hz, 2H), 4.28 (d, J = 5.4 Hz, 2H), 7.25-7.42 (m, 3H), 7.94 (s, 1H), 8.16 (s, 1H), 11.58 (s, 1H); APCI (m/z) 401 (M + H)⁺. |
| 139 | 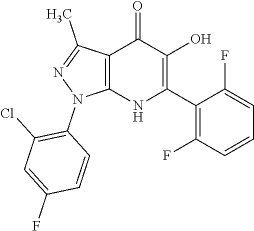 [SMILES: Cc1nn(-c2ccc(F)cc2Cl)c2[nH]c(-c3c(F)cccc3F)c(O)c(=O)c12] | B/Intermediate- 146 | 1-(2-Chloro-4-fluorophenyl)-6-(2,6- difluorophenyl)-5-hydroxy-3-methyl-1H- pyrazolo[3,4-b]pyridin-4(7H)-one; ¹H NMR (300 MHz, DMSO-d₆): δ 2.55 (s, 3H), 7.10-7.28 (m, 2H), 7.30-7.80 (m, 3H), 8.43 (s, 1H), 11.72 (s, 1H); APCI (m/z) 406 (M + H)⁺. |
| 140 | 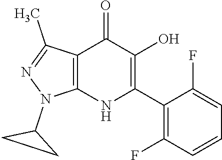 | A/Intermediate- 147 | 1-Cyclopropyl-6-(2,6-difluorophenyl)-3-ethyl- 5-hydroxy-1H-pyrazolo[3,4-b]pyridin-4(7H)- one; ¹H NMR (300 MHz, DMSO-d₆): δ 0.95- 1.08 (m, 4H), 1.25 (t, J = 7.2 Hz, 3H), 2.83 (q, J = 7.2 Hz, 2H), 3.45-3.51 (m, 1H), 7.27 (t, J = 7.8 Hz, 2H), 7.45-7.64 (m, 1H), 8.25 (s, 1H), 11.77 (s, 1H); APCI (m/z) 332 (M + H)⁺. |
| 141 | 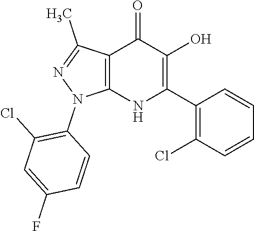 [SMILES: Cc1nn(-c2ccc(F)cc2Cl)c2[nH]c(-c3ccccc3Cl)c(O)c(=O)c12] | B/Intermediate- 148 | 1-(2-Chloro-4-fluorophenyl)-6-(2- chlorophenyl)-5-hydroxy-3-methyl-1H- pyrazolo[3,4-b]pyridin-4(7H)-one; ¹H NMR (300 MHz, DMSO-d₆): δ 2.55 (s, 3H), 7.35-7.60 (m, 6H), 7.67-7.76 (m, 2H), 8.01 (s, 1H), 11.70 (s, 1H); APCI (m/z) 404 (M)⁺. |
| 142 | 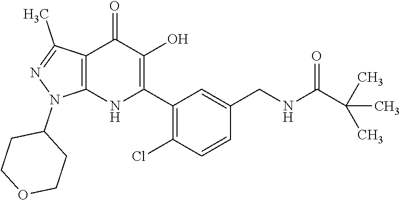 [SMILES: Cc1nn(C2CCOCC2)c2[nH]c(-c3cc(CNC(=O)C(C)(C)C)ccc3Cl)c(O)c(=O)c12] | B/Intermediate- 149 | N-(4-Chloro-3-(5-hydroxy-3-methyl-4-oxo-1- (tetrahydro-2H-pyran-4-yl)-4,7-dihydro-1H- pyrazolo[3,4-b]pyridin-6-yl)benzyl)pivalamide; ¹H NMR (300 MHz, DMSO-d₆): δ 1.11 (s, 9H), 1.77-1.85 (m, 2H), 1.96-2.05 (m, 2H), 2.54 (s, 3H), 3.93-3.97 (m, 2H), 4.26-4.32 (m, 2H), 4.62- 4.66 (m, 1H), 7.25-7.38 (m, 2H), 7.56 (d, J = 7.8 Hz, 1H), 7.89 (s, 1H), 8.20 (s, 1H), 11.59 (s, 1H); APCI (m/z) 473 (M + H)⁺. |
| 143 | 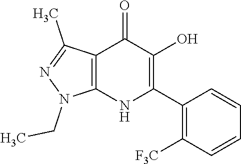 [SMILES: CCn1nc(C)c2c(=O)c(O)c(-c3ccccc3C(F)(F)F)[nH]c21] | B/Intermediate- 150 | 1-Ethyl-5-hydroxy-3-methyl-6-(2- (trifluoromethyl)phenyl)-1H-pyrazolo[3,4- b]pyridin-4(7H)-one; ¹H NMR (300 MHz, DMSO-d₆): δ 1.24 (t, J = 7.2 Hz, 3H), 2.51 (s, 3H), 4.14 (q, J = 7.2 Hz, 2H), 7.57-7.64 (m, 1H), 7.67-7.96 (m, 4H), 11.62 (s, 1H); APCI (m/z) 338 (M + H)⁺. |
| 144 | 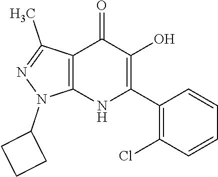 [SMILES: Cc1nn(C2CCC2)c2[nH]c(-c3ccccc3Cl)c(O)c(=O)c12] | B/Intermediate- 151 | 6-(2-Chlorophenyl)-1-cyclobutyl-5-hydroxy-3- methyl-1H-pyrazolo[3,4-b]pyridin-4(7H)-one; ¹H NMR (300 MHz, DMSO-d₆): δ 1.65-1.86 (m, 2H), 2.20-2.40 (m, 2H), 2.42-2.68 (m, 5H, overlapping with DMSO peak), 5.02-5.10 (m, 1H), 7.39-7.74 (m, 4H), 7.90 (s, 1H), 11.54 (s, 1H); APCI (m/z) 330 (M)⁺. |
| 145 | 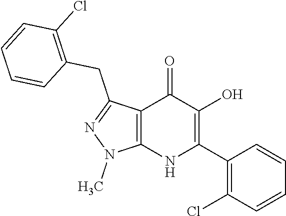 [SMILES: Cn1nc(Cc2ccccc2Cl)c2c(=O)c(O)c(-c3ccccc3Cl)[nH]c21] | B/Intermediate- 152 | 3-(2-Chlorobenzyl)-6-(2-chlorophenyl)-5- hydroxy-1-methyl-1H-pyrazolo[3,4-b]pyridin- 4(7H)-one; ¹H NMR (300 MHz, DMSO-d₆): δ 3.79 (s, 3H), 4.40 (s, 2H), 7.23-7.26 (m, 2H), 7.38-7.54 (m, 5H), 7.62 (s, 1H), 8.00 (s, 1H), 11.72 (s, 1H); APCI (m/z) 401 (M + H)⁺. |
| 146 | 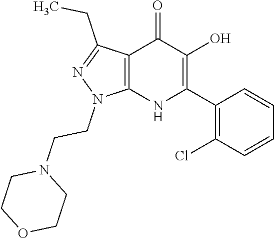 [SMILES: CCc1nn(CCN2CCOCC2)c2[nH]c(-c3ccccc3Cl)c(O)c(=O)c12] | B/Intermediate- 153 | 6-(2-Chlorophenyl)-3-ethyl-5-hydroxy-1-(2- morpholinoethyl)-1H-pyrazolo[3,4-b]pyridin- 4(7H)-one; ¹H NMR (300 MHz, DMSO-d₆): δ 1.27 (t, J = 7.2 Hz, 3H), 2.38-2.47 (m, 4H), 2.69 (t, J = 7.2 Hz, 2H), 2.87 (q, J = 6.9 Hz, 2H), 3.30-3.50 (m, 4H), 4.29 (t, J = 7.2 Hz, 2H), 7.43- 7.59 (m, 3H), 7.62 (br s, 1H), 7.94 (br s, 1H), 11.93 (br s, 1H); ESI (m/z) 403 (M)⁺. |
| 147 | 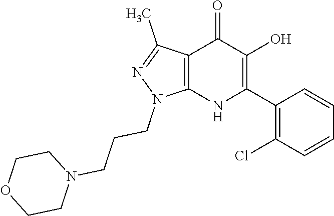 [SMILES: Cc1nn(CCCN2CCOCC2)c2[nH]c(-c3ccccc3Cl)c(O)c(=O)c12] | B/Intermediate- 154 | 6-(2-Chlorophenyl)-5-hydroxy-3-methyl-1-(3- morpholinopropyl)-1H-pyrazolo[3,4-b]pyridin- 4(7H)-one; ¹H NMR (300 MHz, DMSO-d₆): δ 1.80-1.95 (m, 2H), 2.20-2.37 (m, 7H), 3.38-3.54 (m, 6H), 4.10-4.23 (m, 2H), 7.42-7.57 (m, 3H), 7.59-7.64 (m, 1H); ESI (m/z) 403 (M + H)⁺. |
| 148 | 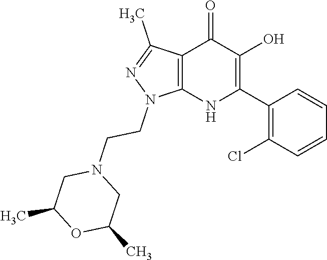 [SMILES: Cc1nn(CCN2C[C@@H](C)O[C@@H](C)C2)c2[nH]c(-c3ccccc3Cl)c(O)c(=O)c12] | B/Intermediate- 155 | 6-(2-Chlorophenyl)-1-(2-((2S,6R)-2,6- dimethylmorpholino)ethyl)-5-hydroxy-3- methyl-1H-pyrazolo[3,4-b]pyridin-4(7H)-one; ¹H NMR (300 MHz, DMSO-d₆): δ 0.96 (d, J = 6.3 Hz, 6H), 1.64 (t, J = 7.8 Hz, 2H), 2.50 (s, 3H), 2.62-2.2.67 (m, 2H), 2.68-2.82 (m, 2H), 3.20-3.50 (m, 2H), 4.28 (t, J = 6.6 Hz, 2H), 7.40- 7.69 (m, 4H), 7.80-7.98 (m, 1H), 11.95 (br s, 1H); ESI (m/z) 417 (M + H)⁺. |
| 149 | 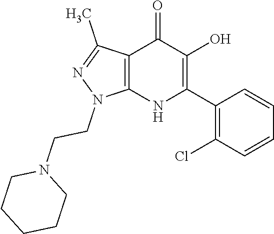 [SMILES: Cc1nn(CCN2CCCCC2)c2[nH]c(-c3ccccc3Cl)c(O)c(=O)c12] | B/Intermediate- 156 | 6-(2-Chlorophenyl)-5-hydroxy-3-methyl-1-(2- (piperidin-1-yl)ethyl)-1H-pyrazolo[3,4- b]pyridin-4(7H)-one; ¹H NMR (300 MHz, DMSO-d₆): δ 1.08-1.34 (m, 6H), 2.40 (s, 3H), 2.45-2.56 (m, 4H), 2.69 (t, J = 6.6 Hz, 2H), 4.27 (t, J = 6.6 Hz, 2H), 7.43-7.53 (m, 3H), 7.56-7.64 (m, 1H); ESI (m/z) 387 (M + H)⁺. |
| 150 | 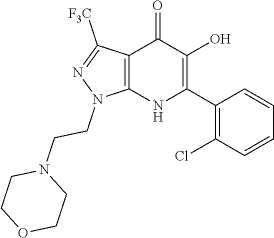 [SMILES: O=c1c(O)c(-c2ccccc2Cl)[nH]c2c1c(C(F)(F)F)nn2CCN1CCOCC1] | B/Intermediate- 157 | 6-(2-Chlorophenyl)-5-hydroxy-1-(2- morpholinoethyl)-3-(trifluoromethyl)-1H- pyrazolo[3,4-b]pyridin-4(7H)-one; ¹H NMR (300 MHz, DMSO-d₆): δ 2.40-2.54 (m, 4H), 2.80 (t, J = 5.7 Hz, 2H), 3.36-2.48 (m, 4H), 4.50 (t, J = 5.7 Hz, 2H), 7.42-7.57 (m, 3H), 7.58-7.64 (m, 1H), ; ESI (m/z) 443 (M + H)⁺. |
| 151 | 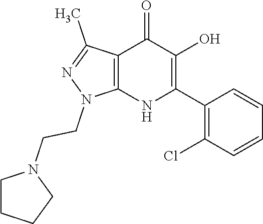 [SMILES: Cc1nn(CCN2CCCC2)c2[nH]c(-c3ccccc3Cl)c(O)c(=O)c12] | B/Intermediate- 158 | 6-(2-Chlorophenyl)-5-hydroxy-3-methyl-1-(2- (pyrrolidin-1-yl)ethyl)-1H-pyrazolo[3,4- b]pyridin-4(7H)-one; ¹H NMR (300 MHz, DMSO-d₆): δ 1.38-1.47 (m, 4H), 2.45-2.55 (m, 4H), 2.90 (t, J = 6.6 Hz, 2H), 3.16 (s, 3H), 4.29 (t, J = 6.6 Hz, 2H), 7.43-7.56 (m, 3H), 7.59 (d, J = 6.9 Hz, 1H); ESI (m/z) 373 (M + H)⁺. |
| 152 | 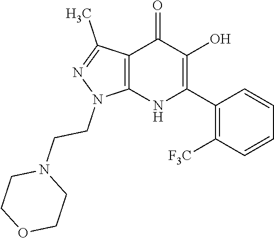 [SMILES: Cc1nn(CCN2CCOCC2)c2[nH]c(-c3ccccc3C(F)(F)F)c(O)c(=O)c12] | B/Intermediate- 159 | 5-Hydroxy-3-methyl-1-(2-morpholinoethyl)-6- (2-(trifluoromethyl)phenyl)-1H-pyrazolo[3,4- b]pyridin-4(7H)-one; ¹H NMR (300 MHz, DMSO-d₆): δ 2.38 (s, 3H), 2.46-2.60 (m, 2H), 2.67 (t, J = 6.6 Hz, 2H), 3.30-3.43 (m, 6H), 4.20- 4.36 (m, 2H), 7.59 (s, 1H), 7.68-7.97 (m, 3H), 12.0 (br. s, 1H); APCI (m/z) 423 (M + H)⁺. |
| 153 | 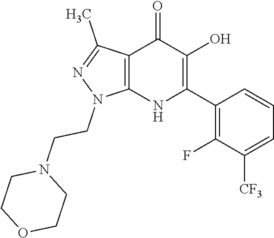 [SMILES: Cc1nn(CCN2CCOCC2)c2[nH]c(-c3cccc(C(F)(F)F)c3F)c(O)c(=O)c12] | B/Intermediate- 160 | 6-(2-fluoro-3-(trifluoromethyl)phenyl)-5- hydroxy-3-methyl-1-(2-morpholinoethyl)-1H- pyrazolo[3,4-b]pyridin-4(7H)-one; ¹H NMR (300 MHz, DMSO-d₆): δ 2.42 (s, 3H), 2.46-2.59 (m, 4H), 2.70 (t, J = 6.6 Hz, 2H), 3.403.49 (m, 4H), 4.29 (t, J = 6.6 Hz, 2H), 7.56 (d, J = 6.9 Hz, 1H), 7.84-8.00 (m, 2H); APCI (m/z) 441 (M + H)⁺. |
| 154 | 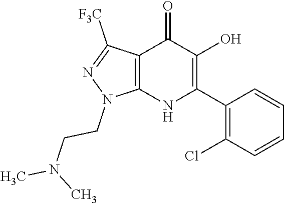 [SMILES: CN(C)CCn1nc(C(F)(F)F)c2c(=O)c(O)c(-c3ccccc3Cl)[nH]c21] | B/Intermediate- 161 | 6-(2-chlorophenyl)-1-(2-(dimethylamino)ethyl)- 5-hydroxy-3-(trifluoromethyl)-1H- pyrazolo[3,4-b]pyridin-4(7H)-one; ¹H NMR (300 MHz, DMSO-d₆): δ 2.33 (s, 6H), 2.99 (t, J = 6.6 Hz, 2H), 4.51 (t, J = 6.6 Hz, 2H), 7.43-7.52 (m, 3H), 7.54-7.58 (m, 1H); APCI (m/z) 401 (M)⁺. |
| 155 | 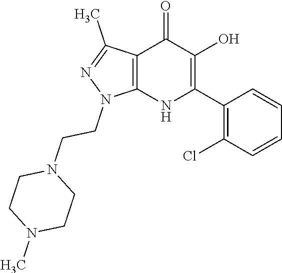 [SMILES: Cc1nn(CCN2CCN(C)CC2)c2[nH]c(-c3ccccc3Cl)c(O)c(=O)c12] | B/Intermediate- 162 | 6-(2-chlorophenyl)-5-hydroxy-3-methyl-1-(2- (4-methylpiperazin-1-yl)ethyl)-1H- pyrazolo[3,4-b]pyridin-4(7H)-one; ¹H NMR (300 MHz, DMSO-d₆): δ 1.99 (s, 6H), 2.30-2.60 (m, 8H), 2.71 (t, J = 6.6 Hz, 2H), 4.28 (t, J = 6.6 Hz, 2H), 7.40-7.58 (m, 3H), 7.62 (d, J = 5.7 Hz, 1H), 8.31 (s, 1H), 12.26 (br. s, 1H). |
| 156 | 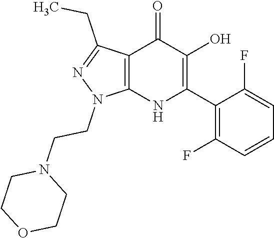 [SMILES: CCc1nn(CCN2CCOCC2)c2[nH]c(-c3c(F)cccc3F)c(O)c(=O)c12] | B/Intermediate- 163 | 6-(2,6-difluorophenyl)-3-ethyl-5-hydroxy-1-(2- morpholinoethyl)-1H-pyrazolo[3,4-b]pyridin- 4(7H)-one; ¹H NMR (300 MHz, DMSO-d₆): δ 1.25 (t, J = 7.2 Hz, 3H), 2.34-2.40 (m, 4H), 2.67 (t, J = 6.6 Hz, 2H), 2.78-2.93 (m, 2H), 3.30-3.50 (m, 4H), 4.20-4.29 (m, 2H), 7.20-7.38 (m, 2H), 7.56-7.64 (m, 1H). |
| 157 | 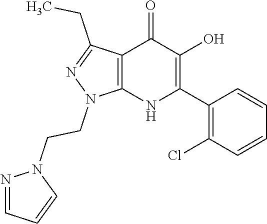 [SMILES: CCc1nn(CCn2cccn2)c2[nH]c(-c3ccccc3Cl)c(O)c(=O)c12] | B/Intermediate- 164 | 1-(2-(1H-pyrazol-1-yl)ethyl)-6-(2-chlorophenyl)- 3-ethyl-5-hydroxy-1H-pyrazolo[3,4-b]pyridin- 4(7H)-one; ¹H NMR (300 MHz, DMSO-d₆): δ 1.27 (t, J = 7.2 Hz, 3H), 2.80-3.00 (m, 2H), 4.47- 4.59 (m, 4H), 6.13 (s, 1H), 7.34-7.62 (m, 6H), 7.84-7.88 (m, 1H), 11.48 (s, 1H); ESI (m/z) 384 (M + H)⁺. |
| 158 | 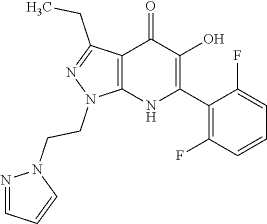 [SMILES: CCc1nn(CCn2cccn2)c2[nH]c(-c3c(F)cccc3F)c(O)c(=O)c12] | B/Intermediate- 165 | 1-(2-(1H-pyrazol-1-yl)ethyl)-6-(2,6- difluorophenyl)-3-ethyl-5-hydroxy-1H- pyrazolo[3,4-b]pyridin-4(7H)-one; ¹H NMR (300 MHz, DMSO-d₆); δ 1.25 (t, J = 7.2 Hz, 3H), 2.80- 3.00 (m, 2H), 4.42-4.60 (m, 4H), 6.09 (s, 1H), 7.25 (t, J = 7.8 Hz, 2H), 7.33-7.36 (m, 2H), 7.58- 7.65 (m, 1H), 8.15-8.20 (br s, 1H), 11.59 (s, 1H); APCI (m/z) 386 (M)⁺. |
| 159 | 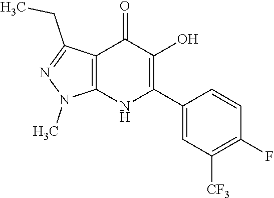 [SMILES: CCc1nn(C)c2[nH]c(-c3ccc(F)c(C(F)(F)F)c3)c(O)c(=O)c12] | B/Intermediate- 166 | 3-Ethyl-6-(4-fluoro-3-(trifluoromethyl)phenyl)- 5-hydroxy-1-methyl-1H-pyrazolo[3,4-b]pyridine- 4(7H)-one; ¹H NMR (300 MHz, DMSO-d₆): δ 1.25 (t, J = 7.5 Hz, 3H), 2.88-2.96 (m, 2H), 3.84 (s, 3H), 7.57-7.75 (m, 1H), 8.00-8.45 (m, 3H), 11.20-11.40 (m, 1H); ESI (m/z) 355 (M)⁺. |
| 160 | 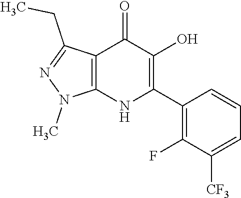 [SMILES: CCc1nn(C)c2[nH]c(-c3cccc(C(F)(F)F)c3F)c(O)c(=O)c12] | B/Intermediate- 167 | 3-Ethyl-6-(2-fluoro-3-(trifluoromethyl)phenyl)- 5-hydroxy-1-methyl-1H-pyrazolo[3,4- b]pyridine-4(7H)-one; ¹H NMR (300 MHz, DMSO-d₆): δ 1.27 (t, J = 7.8 Hz, 3H), 2.88 (q, J = 7.2 Hz, 2H), 3.79 (s, 3H), 7.56 (t, J = 6.9 Hz, 1H), 7.80-8.00 (m, 2H), 8.20-8.36 (m, 1H), 11.62-11.70 (m, 1H); ESI (m/z) 356 (M)⁺. |
| 161 | 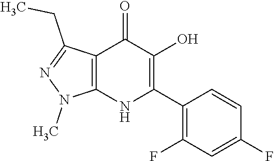 [SMILES: CCc1nn(C)c2[nH]c(-c3ccc(F)cc3F)c(O)c(=O)c12] | B/Intermediate- 168 | 6-(2,4-Difluorophenyl)-3-ethyl-5-hydroxy-1- methyl-1H-pyrazolo[3,4-b]pyridin-4(7H)-one; ¹H NMR (300 MHz, DMSO-d₆): 1.27 (t, J = 7.2 Hz, 3H), 2.88 (q, J = 6.9 Hz, 2H), 3.79 (s, 3H), 7.20-7.30 (m, 1H), 7.40-7.50 (m, 1H), 7.56-7.64 (m, 1H), 8.00-8.05 (m, 1H), 11.56 (s, 1H); ESI (m/z) 306 (M + H). |
| 162 | 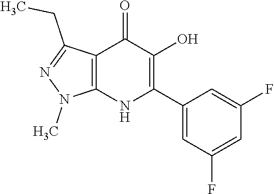 [SMILES: CCc1nn(C)c2[nH]c(-c3cc(F)cc(F)c3)c(O)c(=O)c12] | B/Intermediate- 169 | 6-(3,5-Difluorophenyl)-3-ethyl-5-hydroxy-1- methyl-1H-pyrazolo[3,4-b]pyridine-4(7H)-one; ¹H NMR (300 MHz, DMSO-d₆): δ 1.26 (t, J = 7.2 Hz, 3H), 2.80-3.00 (m, 2H), 3.85 (s, 3H), 7.20-7.80 (m, 3H), 8.26-8.30 (m, 1H), 11.28 (br s, 1H); ESI (m/z) 305 (M + H)⁺. |
| 163 | 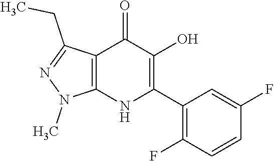 [SMILES: CCc1nn(C)c2[nH]c(-c3cc(F)ccc3F)c(O)c(=O)c12] | B/Intermediate- 170 | 6-(2,5-Difluorophenyl)-3-ethyl-5-hydroxy-1- methyl-1H-pyrazolo[3,4-b]pyridine-4(7H)-one; ¹H NMR (300 MHz, DMSO-d₆): δ 1.27 (t, J = 7.5 Hz, 3H), 2.80-2.97 (m, 2H), 3.79(br s, 3H), 7.20-7.45 (m, 3H), 8.00-8.10 (br s, 1H), 11.50- 11.70 (m, 1H); APCI (m/z) 306 (M + H)⁺. |
| 164 | 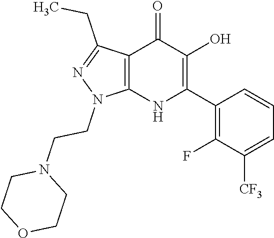 [SMILES: CCc1nn(CCN2CCOCC2)c2[nH]c(-c3cccc(C(F)(F)F)c3F)c(O)c(=O)c12] | B/Intermediate- 171 | 3-Ethyl-6-(2-fluoro-3-(trifluoromethyl)phenyl)- 5-hydroxy-1-(2-morpholinoethyl)-1H- pyrazolo[3,4-b]pyridin-4(7H)-one; ¹H NMR (300 MHz, DMSO-d₆): δ 1.27 (t, J = 7.2 Hz, 3H), 2.38-2.45 (m, 3H), 2.70 (t, J = 7.2 Hz, 2H), 2.89 (q, J = 6.9 Hz, 2H), 3.20-3.58 (m, 5H), 4.30 (t, J = 6.3 Hz, 2H), 7.46-7.62 (m, 1H), 7.87-7.98 (m, 2H). |
| 165 | 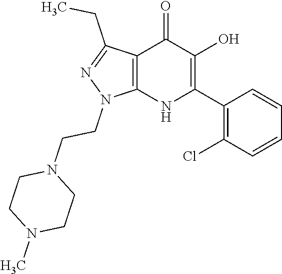 [SMILES: CCc1nn(CCN2CCN(C)CC2)c2[nH]c(-c3ccccc3Cl)c(O)c(=O)c12] | B/Intermediate- 172 | 6-(2-Chlorophenyl)-3-ethyl-5-hydroxy-1-(2-(4- methylpiperazin-1-yl)ethyl)-1H-pyrazolo[3,4- b]pyridine-4(7H)-one; ¹H NMR (300 MHz, DMSO-d₆): δ 1.25 (t, J = 7.2 Hz, 3H), 1.96 (s, 3H), 1.98-2.28 (m, 4H), 2.40-2.52 (m, 4H), 2.70 (t, J = 6.3 Hz, 2H), 2.85 (q, J = 7.5 Hz, 2H), 4.27 (t, J = 6.6 Hz, 2H), 7.40-7.58 (m, 3H), 7.60 (d, J = 9.0 Hz, 1H); APCI (m/z) 416 (M + H)⁺. |
| 166 | 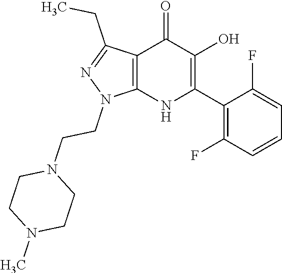 [SMILES: CCc1nn(CCN2CCN(C)CC2)c2[nH]c(-c3c(F)cccc3F)c(O)c(=O)c12] | B/Intermediate- 173 | 6-(2,6-Difluorophenyl)-3-ethyl-5-hydroxy-1-(2- (4-methylpiperazin-1-yl)ethyl)-1H- pyrazolo[3,4-b]pyridine-4(7H)-one; ¹H NMR (300 MHz, DMSO-d₆): δ 1.25 (t, J = 7.2 Hz, 3H), 2.01 (s, 3H), 2.03-2.24 (m, 4H), 2.40-2.50 (m, 4H), 2.70 (t, J = 6.0 Hz, 2H), 2.86 (q, J = 6.9 Hz, 2H), 4.26 (t, J = 6.6 Hz, 2H), 7.20-7.35 (m, 2H), 7.55-7.65 (m, 1H). |
| 167 | 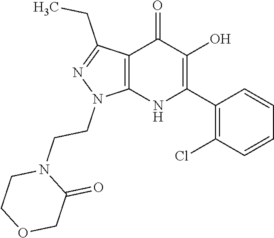 [SMILES: CCc1nn(CCN2CCOCC2=O)c2[nH]c(-c3ccccc3Cl)c(O)c(=O)c12] | B/Intermediate- 174 | 4-(2-(6-(2-Chlorophenyl)-3-ethyl-5-hydroxy-4- oxo-4,7-dihydro-1H-pyrazolo[3,4-b]pyridin-1- yl)ethyl)morpholin-3-one; ¹H NMR (300 MHz, DMSO-d₆): δ 1.27 (t, J = 7.2 Hz, 3H), 2.80-3.00 (m, 2H), 3.02-3.20 (m, 2H), 3.58-3.75 (m, 4H), 3.93 (s, 2H), 4.26-4.44 (m, 2H), 7.36-7.68 (m, 4H), 7.89-8.00 (m, 1H), 11.68 (s, 1H); ESI (m/z) 417 (M + H)⁺. |
| 168 | 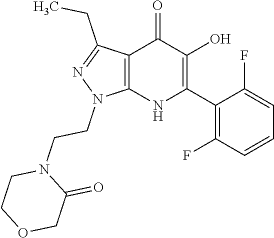 [SMILES: CCc1nn(CCN2CCOCC2=O)c2[nH]c(-c3c(F)cccc3F)c(O)c(=O)c12] | B/Intermediate- 175 | 4-(2-(6-(2,6-Difluorophenyl)-3-ethyl-5- hydroxy-4-oxo-4,7-dihydro-1H-pyrazolo[3,4- b]pyridin-1-yl)ethyl)morpholin-3-one; ¹H NMR (300 MHz, DMSO-d₆): δ 1.25 (t, J = 7.8 Hz, 3H), 2.80-3.00 (m, 2H), 3.02-3.10 (m, 1H), 3.15 (d, J = 4.8 Hz, 2H), 3.58-3.68 (m, 2H), 3.90 (s, 1H), 3.80 (s, 1H), 4.04-4.08 (m, 1H), 4.29-4.42 (m, 2H), 7.27 (t, J = 7.8 Hz, 2H), 7.54-7.67 (m, 1H), 8.15 (s, 1H), 11.74 (s, 1H); ESI (m/z) 419 (M + H)⁺. |
| 169 | 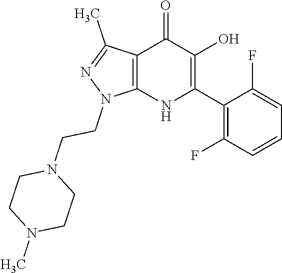 [SMILES: Cc1nn(CCN2CCN(C)CC2)c2[nH]c(-c3c(F)cccc3F)c(O)c(=O)c12] | B/Intermediate- 176 | 6-(2,6-Difluorophenyl)-5-hydroxy-3-methyl-1- (2-(4-methylpiperazin-1-yl)ethyl)-1H- pyrazolo[3,4-b]pyridin-4(7H)-one; ¹H NMR (500 MHz, DMSO-d₆): δ 2.03 (s, 3H), 2.02-2.30 (m, 4H), 2.40-2.45 (m, 4H), 2.47 (s, 3H), 2.70 (t, J = 5.2 Hz, 2H), 4.26 (t, J = 5.2 Hz, 2H), 7.20- 7.32 (m, 2H), 7.56-7.64 (m, 1H), 8.18 (br s, 1H).; APCI (m/z) 404 (M + H)⁺. |
| 170 | 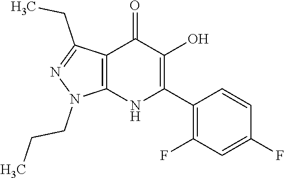 [SMILES: CCCn1nc(CC)c2c(=O)c(O)c(-c3ccc(F)cc3F)[nH]c21] | B/Intermediate- 177 | 6-(2,4-Difluorophenyl)-3-ethyl-5-hydroxy-1- propyl-1H-pyrazolo[3,4-b]pyridin-4(7H)-one; ¹H NMR (500 MHz, DMSO-d₆): δ 0.83 (t, J = 7.5 Hz, 3H), 1.27 (t, J = 7.5 Hz, 3H), 1.68-1.76 (m, 2H), 2.82-2.92 (m, 2H), 4.11 (br. s, 2H), 7.20-7.29 (m1H), 7.40-7.47 (m, 1H), 7.60-7.64 (m, 1H), 7.97-8.10 (m, 1H).; APCI (m/z) 334 (M + H)⁺. |
| 171 | 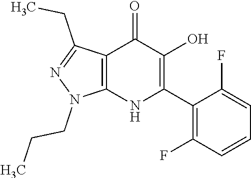 [SMILES: CCCn1nc(CC)c2c(=O)c(O)c(-c3c(F)cccc3F)[nH]c21] | B/Intermediate- 178 | 6-(2,4-Difluorophenyl)-3-ethyl-5-hydroxy-1- propyl-1H-pyrazolo[3,4-b]pyridin-4(7H)-one; ¹H NMR (500 MHz, DMSO-d₆): δ 0.82 (t, J = 7.5 Hz, 3H), 1.27 (t, J = 7.5 Hz, 3H), 1.67-1.76 (m, 2H), 2.86 (t, J = 7.0 Hz, 2H), 4.08 (t, J = 7.0 Hz, 2H), 7.28 (t, J = 8.0 Hz, 2H), 7.52-7.67 (m, 1H), 8.21 (s, 1H), 11.63 (s, 1H).; APCI (m/z) 334 (M + H)⁺. |
| 172 | 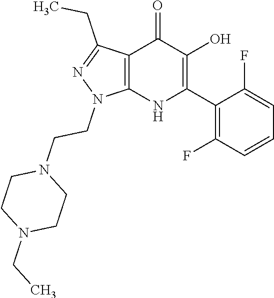 [SMILES: CCc1nn(CCN2CCN(CC)CC2)c2[nH]c(-c3c(F)cccc3F)c(O)c(=O)c12] | B/Intermediate- 179 | 6-(2,6-Difluorophenyl)-3-ethyl-1-(2-(4- ethylpiperazin-1-yl)ethyl)-5-hydroxy-1H- pyrazolo[3,4-b]pyridin-4(7H)-one; ¹H NMR (400 MHz, DMSO-d₆): δ 0.92 (t, J = 7.2 Hz, 3H), 1.28 (t, J = 7.2 Hz, 3H), 2.14-2.60 (m, 10H), 2.72 (t, J = 6.0 Hz, 2H), 2.88 (q, J = 7.6 Hz, 2H), 4.28 (t, J = 6.0 Hz, 2H), 7.27 (t, J = 7.6 Hz, 2H), 7.58-7.63 (m, 1H), 8.14-8.27 (br s, 1H); APCI (m/z) 433 (M + H)⁺. |
| 173 | 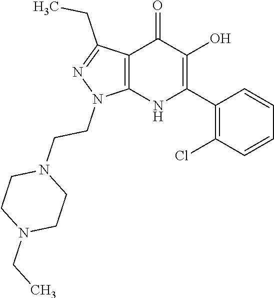 [SMILES: CCc1nn(CCN2CCN(CC)CC2)c2[nH]c(-c3ccccc3Cl)c(O)c(=O)c12] | B/Intermediate- 180 | 6-(2-Chlorophenyl)-3-ethyl-1-(2-(4- ethylpiperazin-1-yl)ethyl)-5-hydroxy-1H- pyrazolo[3,4-b]pyridin-4(7H)-one; ¹H NMR (400 MHz, DMSO-d₆): δ 0.90 (t, J = 7.2 Hz, 3H), 1.28 (t, J = 7.6 Hz, 3H), 1.97-2.61 (m, 10H), 2.72 (t, J = 6.0 Hz, 2H), 2.88 (q, J = 7.6 Hz, 2H), 4.28 (t, J = 6.0 Hz, 2H), 7.27 (t, J = 7.6 Hz, 2H), 7.58-7.63 (m, 1H), 8.14-8.27 (br s, 1H); ESI (m/z) 431 (M + H)⁺. |
| 174 | 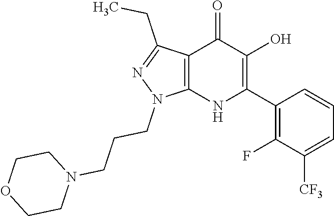 [SMILES: CCc1nn(CCCN2CCOCC2)c2[nH]c(-c3cccc(C(F)(F)F)c3F)c(O)c(=O)c12] | B/Intermediate- 181 | 3-Ethyl-6-(2-fluoro-3-(trifluoromethyl)phenyl)- 5-hydroxy-1-(3-morpholinopropyl)-1H- pyrazolo[3,4-b]pyridin-4(7H)-one; ¹H NMR (400 MHz, DMSO-d₆): δ 1.28 (t, J = 6.8 Hz, 3H), 1.91 (t, J = 6.8 Hz, 2H), 2.26 (t, J = 6.8 Hz, 4H), 2.89 (q, J = 7.6 Hz, 2H), 3.45 (t, J = 4.8 Hz, 7H), 4.21 (t, J = 6.8 Hz, 2H), 7.57 (t, J = 7.6 Hz, 1H), 7.87-7.93 (m, 2H); APCI (m/z) 469 (M + H)⁺. |
| 175 | 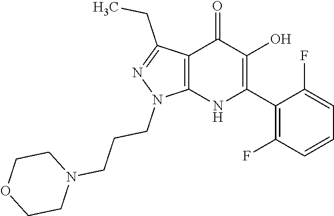 [SMILES: CCc1nn(CCCN2CCOCC2)c2[nH]c(-c3c(F)cccc3F)c(O)c(=O)c12] | B/Intermediate- 182 | 6-(2,6-Difluorophenyl)-3-ethyl-5-hydroxy-1-(3- morpholinopropyl)-1H-pyrazolo[3,4-b]pyridin- 4(7H)-one; ¹H NMR (400 MHz, DMSO-d₆): δ 1.27 (t, J = 7.6 Hz, 3H), 1.889 (t, J = 6.8 Hz, 2H), 2.22-2.28 (m, 4H), 2.88 (q, J = 7.2 Hz, 2H), 3.33-3.52 (m, 6H), 4.18-4.20 (br s, 2H), 7.20- 7.29 (m, 2H), 7.60-7.65 (m, 1H), 8.20-8.40 (m, 1H); APCI (m/z) 419 (M + H)⁺. |
| 176 | 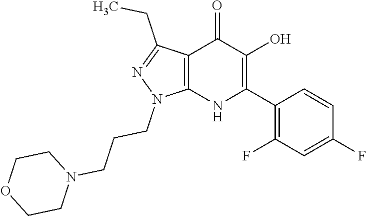 [SMILES: CCc1nn(CCCN2CCOCC2)c2[nH]c(-c3ccc(F)cc3F)c(O)c(=O)c12] | B/Intermediate- 183 | 6-(2,4-Difluorophenyl)-3-ethyl-5-hydroxy-1-(3- morpholinopropyl)-1H-pyrazolo[3,4-b]pyridin- 4(7H)-one; ¹H NMR (400 MHz, DMSO-d₆): δ 1.27 (t, J = 7.6 Hz, 3H), 1.89 (t, J = 6.8 Hz, 2H), 2.23-2.29 (m, 4H), 2.88 (q, J = 7.6 Hz, 2H), 3.30-3.48 (m, 6H), 4.20 (t, J = 6.0 Hz, 2H), 7.26 (t, J = 8.0 Hz, 1H), 7.38-7.53 (m, 1H), 7.61 (q, J = 8.0 Hz, 1H), 8.09 (br. s, 1H).; APCI (m/z) 419 (M + H)⁺. |
| 177 | 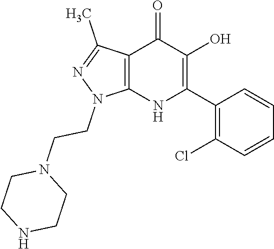 [SMILES: Cc1nn(CCN2CCNCC2)c2[nH]c(-c3ccccc3Cl)c(O)c(=O)c12] | B/Intermediate- 184 | 6-(2-Chlorophenyl)-5-hydroxy-3-methyl-1-(2- (piperazin-1-yl)ethyl)-1H-pyrazolo[3,4- b]pyridin-4(7H)-one; ¹H NMR (300 MHz, DMSO-d₆): δ 2.40-2.65 (m, 8H), 2.70-2.76 (m, 2H), 2.90-2.94 (m, 4H), 4.23-4.27 (m, 2H), 7.46- 7.62 (m, 4H), 8.46 (br, s, 1H). ESI (m/z) 388 (M + H)+. |
| 178 | 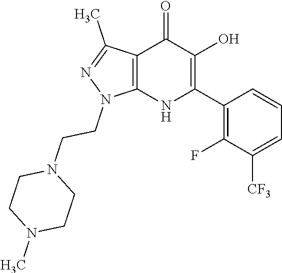 [SMILES: Cc1nn(CCN2CCN(C)CC2)c2[nH]c(-c3cccc(C(F)(F)F)c3F)c(O)c(=O)c12] | B/Intermediate- 185 | 6-(2-fluoro-3-(trifluoromethyl)phenyl)-5- hydroxy-3-methyl-1-(2-(4-methylpiperazin-1- yl)ethyl)-1H-pyrazolo[3,4-b]pyridin-4(7H)-one; ¹H NMR (300 MHz, DMSO-d₆): δ 2.08 (s, 3H), 2.20-2.51 (m, 11H), 2.72 (t, J = 8.0 Hz, 2H), 4.28 (t, J = 6.0 Hz, 2H), 7.55 (t, J = 7.6 Hz, 4H), 7.85- 7.92 (m, 2H). ESI (m/z) 454 (M + H)+. |
| 179 | 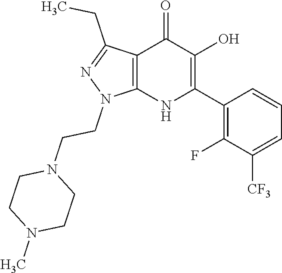 [SMILES: CCc1nn(CCN2CCN(C)CC2)c2[nH]c(-c3cccc(C(F)(F)F)c3F)c(O)c(=O)c12] | B/Intermediate- 186 | 3-Ethyl-6-(2-fluoro-3-(trifluoromethyl)phenyl)- 5-hydroxy-1-(2-(4-methylpiperazin-1-yl)ethyl)- 1H-pyrazolo[3,4-b]pyridin-4(7H)-one; ¹H NMR (300 MHz, DMSO-d₆): δ 1.27 (t, J = 7.2 Hz, 3H), 2.06-2.53 (m, 11H), 2.70-2.74 (m, 2H), 2.85- 2.91 (m, 2H), 4.27-4.34 (m, 2H), 7.58 (t, J = 7.2 Hz, 4H), 7.87-7.92 (m, 2H). ESI (m/z) 468 (M + H)+. |
| 180 | 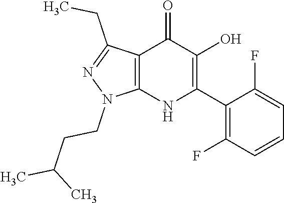 [SMILES: CCc1nn(CCC(C)C)c2[nH]c(-c3c(F)cccc3F)c(O)c(=O)c12] | B/Intermediate- 187 | 6-(2,6-Difluorophenyl)-3-ethyl-5-hydroxy-1- isopentyl-1H-pyrazolo[3,4-b]pyridin-4(7H)- one; ¹H NMR (400 MHz, DMSO-d₆): δ 0.88 (d, J = 6.0 Hz, 6H), 1.27-1.29 (m, 3H), 1.49-1.60 (m, 1H), 1.62-1.68 (m, 2H), 2.84-2.94 (m, 2H), 4.14 (t, J = 7.2 Hz, 2H), 7.29 (t, J = 8.0 Hz, 2H), 7.60-7.70 (m, 1H), 8.24 (s, 1H), 11.65 (s, 1H); ESI (m/z) 362 (M + H)+. |
| 181 | 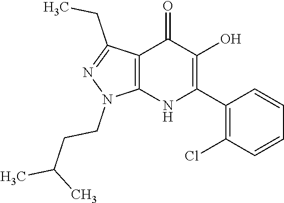 [SMILES: CCc1nn(CCC(C)C)c2[nH]c(-c3ccccc3Cl)c(O)c(=O)c12] | B/Intermediate- 188 | 6-(2-Chlorophenyl)-3-ethyl-5-hydroxy-1- isopentyl-1H-pyrazolo[3,4-b]pyridin-4(7H)- one; ¹H NMR (400 MHz, DMSO-d₆): δ 0.88 (d, J = 6.0 Hz, 6H), 1.48 (t, J = 6.8 Hz, 2H), 1.59- 1.65 (m, 3H), 2.84-2.89 (m, 2H), 4.15 (t, J = 7.2 Hz, 2H), 7.42-7.63 (m, 5H), 7.88 (s, 1H), 11.56 (s, 1H). |
| 182 | 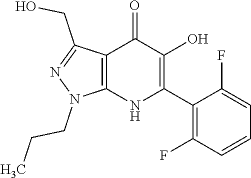 [SMILES: CCCn1nc(CO)c2c(=O)c(O)c(-c3c(F)cccc3F)[nH]c21] | B/Intermediate- 189 | 6-(2,6-Difluorophenyl)-5-hydroxy-3- (hydroxymethyl)-1-propyl-1H-pyrazolo[3,4- b]pyridin-4(7H)-one; ¹H NMR (400 MHz, DMSO-d₆): δ 0.82 (t, J = 7.60 Hz, 3H), 1.75-1.77 (m, 2H), 4.08-4.17 (m, 2H), 4.71-4.84 (m, 2H), 5.97-6.0 (m, 1H), 7.20-7.30 (m, 2H), 7.50-7.68 (m, 1H), 8.60 (s, 1H), 12.12 (br s, 1H). |
| 183 | 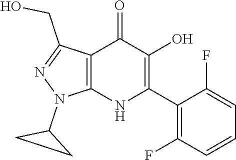 [SMILES: O=c1c(O)c(-c2c(F)cccc2F)[nH]c2c1c(CO)nn2C1CC1] | B/Intermediate- 190 | 1-Cyclopropyl-6-(2,6-difluorophenyl)-5- hydroxy-3-(hydroxymethyl)-1H-pyrazolo[3,4- b]pyridin-4(7H)-one; ¹H NMR (300 MHz, DMSO-d₆): ¹H NMR (400 MHz, DMSO-d₆)): δ 1.03-1.07 (m, 4H), 3.50-3.60 (m, 1H), 4.60-4.70 (br s, 2H), 4.80-4.90 (m, 1H), 5.90-6.0 (m, 1H), 7.21-7.29 (m, 2H), 7.55-7.65 (m, 1H), 12.26 (br s, 1H); ESI (m/z) 334 (M + H)+. |
| 184 | 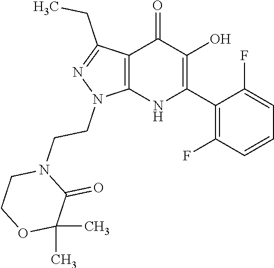 [SMILES: CCc1nn(CCN2CCOC(C)(C)C2=O)c2[nH]c(-c3c(F)cccc3F)c(O)c(=O)c12] | B/Intermediate- 191 | 4-(2-(6-(2,6-Difluorophenyl)-3-ethyl-5- hydroxy-4-oxo-4,7-dihydro-1H-pyrazolo[3,4- b]pyridin-1-yl)ethyl)-2,2-dimethylmorpholin-3- one; ¹H NMR (300 MHz, DMSO-d₆): ¹H NMR (400 MHz, DMSO-d₆): δ 1.12 (s, 6H), 1.23 (t, J = 8.0 Hz, 3H), 2.84-2.86 (m, 2H), 3.0-3.10 (m, 2H), 3.93-3.98 (m, 4H), 4.31-4.35 (m, 2H), 7.15- 7.22 (m, 2H), 7.59 (br s, 1H); ESI (m/z) 447 (M + H)⁺. |
| 185 | 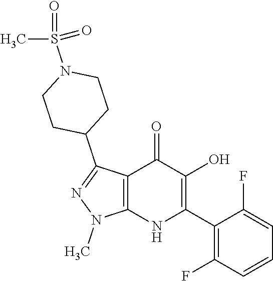 [SMILES: Cn1nc(C2CCN(S(C)(=O)=O)CC2)c2c(=O)c(O)c(-c3c(F)cccc3F)[nH]c21] | B/Intermediate- 192 | 6-(2,6-Difluorophenyl)-5-hydroxy-1-methyl-3- (1-(methylsulfonyl)piperidin-4-yl)-1H- pyrazolo[3,4-b]pyridin-4(7H)-one; ¹H NMR (400 MHz, DMSO-d₆): δ 1.93-1.99 (m, 2H), 2.07-2.10 (m, 2H), 2.87-2.92 (m, 5H), 3.16-3.34 (m, 1H), 3.67 (d, J = 12 Hz, 2H), 3.81 (s, 3H), 7.29 (t, J = 8.0 Hz, 2H), 7.63-7.66 (m, 1H), 8.24 (s, 1H), 11.82 (br s, 1H); ESI (m/z) 439 (M + H)⁺. |
| 186 | 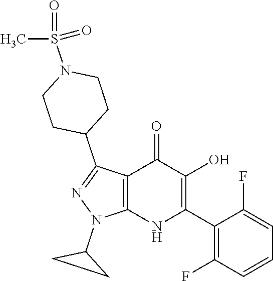 [SMILES: CS(=O)(=O)N1CCC(c2nn(C3CC3)c3[nH]c(-c4c(F)cccc4F)c(O)c(=O)c23)CC1] | B/Intermediate- 193 | 1-Cyclopropyl-6-(2,6-difluorophenyl)-5- hydroxy-3-(1-(methylsulfonyl)piperidin-4-yl)- 1H-pyrazolo[3,4-b]pyridin-4(7H)-one; ¹H NMR (400 MHz, DMSO-d₆): δ 0.99-1.97 (m, 4H), 1.92-2.06 (m, 4H), 2.85-2.91 (m, 5H), 3.12-3.15 (m, 1H), 3.50-3.51 (m, 1H), 3.64 (d, J= 12.0 Hz, 2H), 7.28 (t, J = 8.0 Hz, 2H), 7.61-7.65 (m, 1H), 8.25 (s, 1H), 11.86 (s, 1H). |
| 187 | 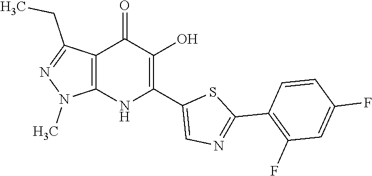 [SMILES: CCc1nn(C)c2[nH]c(-c3cnc(-c4ccc(F)cc4F)s3)c(O)c(=O)c12] | B/Intermediate- 194 | 6-(2-(2,4-Difluorophenyl)thiazol-5-yl)-3-ethyl- 5-hydroxy-1-methyl-1H-pyrazolo[3,4- b]pyridin-4(7H)-one; ¹H NMR (400 MHz, DMSO-d₆): δ 1.28 (t, J = 7.6 Hz, 3H), 2.92 (q, J = 7.2 Hz, 2H), 3.90 (s, 3H), 7.32 (dt, J₁ = 2.4 Hz, J₂ = 8.4 Hz, 1H), 7.56 (dt, J₁ = 2.4 Hz, J₂ = 11.2 Hz, 1H), 8.35 (dd, J₁ = 8.0 Hz, J₂ = 15.6 Hz, 1H), 8.79 (s, 1H), 8.86 (br s, 1H), 10.79 (br s, 1H); ESI (m/z) 389 (M + H)⁺. |
| 188 | 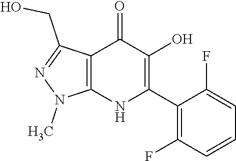 [SMILES: Cn1nc(CO)c2c(=O)c(O)c(-c3c(F)cccc3F)[nH]c21] | B/Intermediate- 195 | 6-(2,6-Difluorophenyl)-5-hydroxy-3- (hydroxymethyl)-1-methyl-1H-pyrazolo[3,4- b]pyridin-4(7H)-one; ¹H NMR (400 MHz, DMSO-d₆): δ 3.85 (s, 3H), 4.72-4.76 (m, 2H), 5.80-5.99 (m, 1H), 7.20-7.30 (m, 2H), 7.60-7.70 (m, 1H), 8.59 (s, 1H), 12.19 (br s, 1H); APCI (m/z) 308 (M + H)⁺. |
| 189 | 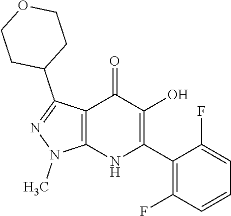 [SMILES: Cn1nc(C2CCOCC2)c2c(=O)c(O)c(-c3c(F)cccc3F)[nH]c21] | B/Intermediate- 196 | 6-(2,6-Difluorophenyl)-5-hydroxy-1-methyl-3- (tetrahydro-2H-pyran-4-yl)-1H-pyrazolo[3,4- b]pyridin-4(7H)-one; ¹H NMR (400 MHz, DMSO-d₆): δ 1.88-1.99 (m, 4H), 3.29-3.34 (m, 1H), 3.43-3.50 (m, 2H), 3.86 (s, 3H), 3.95 (d, J = 10.8 Hz, 2H), 7.28 (t, J = 7.6 Hz, 2H), 7.62- 7.64 (m, 1H), 8.21-8.23 (m, 1H), 11.07 (br s, 1H); ESI (m/z) 362 (M + H)⁺. |
| 190 | 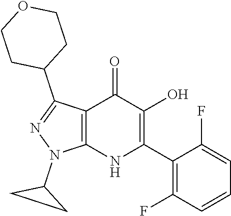 [SMILES: O=c1c(O)c(-c2c(F)cccc2F)[nH]c2c1c(C1CCOCC1)nn2C1CC1] | B/Intermediate- 197 | 1-Cyclopropyl-6-(2,6-difluorophenyl)-5- hydroxy-3-(tetrahydro-2H-pyran-4-yl)-1H- pyrazolo[3,4-b]pyridin-4(7H)-one; ¹H NMR (400 MHz, DMSO-d₆): δ 1.01-1.05 (m, 4H), 1.83-1.95 (m, 4H), 3.16-3.27 (m, 2H), 3.33-3.49 (m, 2H), 3.93 (d, J = 10.4 Hz, 2H), 7.27 (t, J = 8.0 Hz, 2H), 7.60-7.66 (m, 1H), 8.21 (s, 1H), 11.81 (s, 1H); ESI (m/z) 388 (M + H)⁺. |
| 193 | 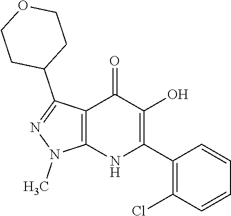 [SMILES: Cn1nc(C2CCOCC2)c2c(=O)c(O)c(-c3ccccc3Cl)[nH]c21] | B/Intermediate- 198 | 6-(2-Chlorophenyl)-5-hydroxy-1-methyl-3- (tetrahydro-2H-pyran-4-yl)-1H-pyrazolo[3,4- b]pyridin-4(7H)-one; ¹H NMR (400 MHz, DMSO-d₆): δ 1.88-1.95 (m, 4H), 3.27-3.30 (m, 1H), 3.43-3.49 (m, 2H), 3.80 (s, 3H), 3.95 (d, J = 10.8 Hz, 2H), 7.48-7.55 (m, 3H), 7.64 (d, J = 7.6 Hz, 1H), 7.86 (s, 1H), 11.66 (s, 1H). |
| 194 | 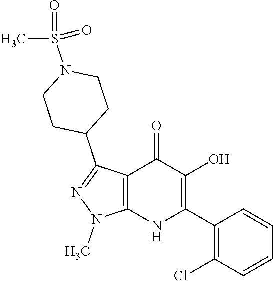 [SMILES: Cn1nc(C2CCN(S(C)(=O)=O)CC2)c2c(=O)c(O)c(-c3ccccc3Cl)[nH]c21] | B/Intermediate- 199 | 6-(2-chlorophenyl)-5-hydroxy-1-methyl-3-(1- (methylsulfonyl)piperidin-4-yl)-1H- pyrazolo[3,4-b]pyridin-4(7H)-one; ¹H NMR (400 MHz, DMSO-d₆): δ 1.92-1.98 (m, 4H), 2.84-2.89 (m, 4H), 3.15 (s, 2H), 3.49-3.67 (m, 2H), 3.80 (s, 3H), 7.47-7.52 (m, 3H), 7.91-7.63 (m, 2H). |
| 198 | 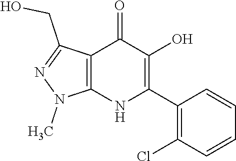 [SMILES: Cn1nc(CO)c2c(=O)c(O)c(-c3ccccc3Cl)[nH]c21] | B/Intermediate- 200 | 6-(2-Chlorophenyl)-5-hydroxy-3- (hydroxymethyl)-1-methyl-1H-pyrazolo[3,4- b]pyridin-4(7H)-one; ¹H NMR (400 MHz, DMSO-d₆): δ 3.84 (s, 3H), 4.73 (s, 2H), 6.16 (s, 1H), 7.46-7.63 (m, 4H), 8.28 (s, 1H), 12.04 (br s, 1H); ESI (m/z) 306 (M + H)⁺. |
| 200 | 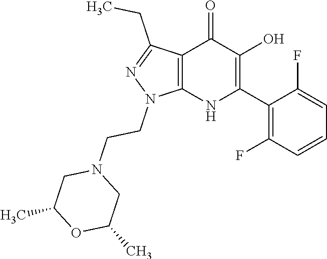 [SMILES: CCc1nn(CCN2C[C@@H](C)O[C@@H](C)C2)c2[nH]c(-c3c(F)cccc3F)c(O)c(=O)c12] | B/Intermediate- 201 | 6-(2,6-Difluorophenyl)-1-(2-((2R,6S)-2,6- dimethylmorpholino)ethyl)-3-ethyl-5-hydroxy- 1H-pyrazolo[3,4-b]pyridin-4(7H)-one; ¹H NMR (400 MHz, DMSO-d₆): δ 0.92 (d, J = 6.4 Hz, 6H), 1.27 (t, J = 7.2 Hz, 3H), 1.64 (t, J = 9.6 Hz, 2H), 2.67 (t, J = 10.4 Hz, 2H), 2.75 (d, J = 10.4 Hz, 2H), 2.88 (q, J = 7.2 Hz, 2H), 3.30-3.37 (m, 2H), 4.28 (br s, 2H), 7.26-7.30 (m, 2H), 7.60- 7.64 (m, 1H), 8.25 (br s, 1H), 11.95 (br s, 1H); ESI (m/z) 433 (M + H)⁺. |
| 202 | 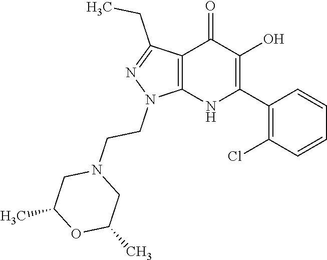 [SMILES: CCc1nn(CCN2C[C@@H](C)O[C@@H](C)C2)c2[nH]c(-c3ccccc3Cl)c(O)c(=O)c12] | B/Intermediate- 202 | 6-(2-Chlorophenyl)-1-(2-((2R,6S)-2,6- dimethylmorpholino)ethyl)-3-ethyl-5-hydroxy- 1H-pyrazolo[3,4-b]pyridin-4(7H)-one; ¹H NMR (400 MHz, DMSO-d₆): δ 0.96 (d, J = 6.4 Hz, 6H), 1.27 (t, J = 7.6 Hz, 3H), 1.65 (t, J = 10.8 Hz, 2H), 2.69 (t, J = 6.4 Hz, 2H), 2.77 (d, J = 10.4 Hz, 2H), 2.87 (q, J = 7.6 Hz, 2H), 3.22-3.32 (m, 4H), 4.30 (t, J = 6.0 Hz, 2H), 7.49-7.59 (m, 3H), 7.62-7.64 (m, 1H), 7.87 (br s, 1H), 12.04 (br s, 1H); ESI (m/z) 431 (M + H)⁺. |
| 203 | 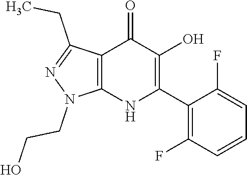 [SMILES: CCc1nn(CCO)c2[nH]c(-c3c(F)cccc3F)c(O)c(=O)c12] | B/Intermediate- 203 | 6-(2,6-Difluorophenyl)-3-ethyl-5-hydroxy-1-(2- hydroxyethyl)-1H-pyrazolo[3,4-b]pyridin- 4(7H)-one; ¹H NMR (400 MHz, DMSO-d₆): δ 1.29 (t, J = 7.6 Hz, 3H), 2.88 (q, J = 7.6 Hz, 2H), 3.73 (s, 2H), 4.20 (s, 2H), 4.88 (br s, 1H), 7.25- 7.27 (m, 2H), 7.61-7.63 (m, 1H), 8.19 (br s, 1H), 11.68 (s, 1H); ESI (m/z) 336 (M + H)⁺. |
| 204 | 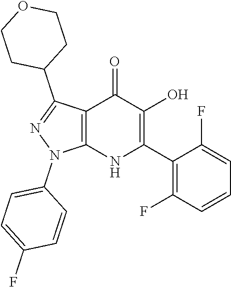 [SMILES: O=c1c(O)c(-c2c(F)cccc2F)[nH]c2c1c(C1CCOCC1)nn2-c1ccc(F)cc1] | B/Intermediate- 204 | 6-(2,6-Difluorophenyl)-1-(4-fluorophenyl)-5- hydroxy-3-(tetrahydro-2H-pyran-4-yl)-1H- pyrazolo[3,4-b]pyridin-4(7H)-one; ¹H NMR (400 MHz, DMSO-d₆): δ 1.95-2.05 (m, 4H), 3.37-3.56 (m, 3H), 4.00 (d, J = 10.8 Hz, 2H), 7.19-7.24 (m, 3H), 7.34 (t, J = 8.8 Hz, 2H), 7.52-7.60 (m, 1H), 8.21 (s, 2H), 11.83-11.85 (br s, 1H). |
| 205 | 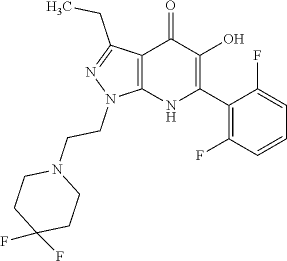 [SMILES: CCc1nn(CCN2CCC(F)(F)CC2)c2[nH]c(-c3c(F)cccc3F)c(O)c(=O)c12] | B/Intermediate- 205 | 6-(2,6-Difluorophenyl)-1-(2-(4,4- difluoropiperidin-1-yl)ethyl)-3-ethyl-5- hydroxy-1H-pyrazolo[3,4-b]pyridin-4(7H)-one; ¹H NMR (400 MHz, DMSO-d₆): δ 1.27 (t, J = 7.2 Hz, 3H), 1.75-1.85 (m, 4H), 2.49-2.55 (m, 2H), 2.75-2.89 (m, 4H), 3.38-3.41 (m, 2H), 4.26 (br s, 2H), 7.27-7.32 (m, 2H), 7.61-7.65 (m, 1H), 8.24 (br s, 1H), 11.76 (br s, 1H). |
| 206 | 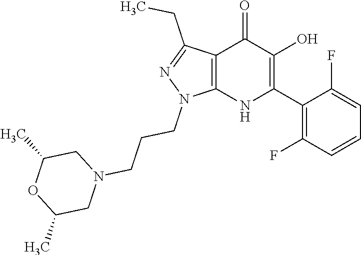 [SMILES: CCc1nn(CCCN2C[C@@H](C)O[C@@H](C)C2)c2[nH]c(-c3c(F)cccc3F)c(O)c(=O)c12] | B/Intermediate- 206 | 6-(2,6-Difluorophenyl)-1-(3-((2R,6S)-2,6- dimethylmorpholino)propyl)-3-ethyl-5- hydroxy-1H-pyrazolo[3,4-b]pyridin-4(7H)-one; ¹H NMR (400 MHz, DMSO-d₆): δ 0.97 (d, J = 6.4 Hz, 6H), 1.23-1.34 (m, 3H), 1.45 (t, J = 10.4 Hz, 2H), 1.88-1.91 (m, 2H), 2.23 (t, J = 6.8 Hz, 2H), 2.58 (d, J = 10.8 Hz, 2H), 2.88 (q, J = 7.2 Hz, 2H), 3.32-3.39 (m, 2H), 4.18 (m, 2H), 7.27 (t, J = 6.8 Hz, 2H), 7.61-7.70 (m, 1H), 8.24 (br s, 1H). |
| 207 | 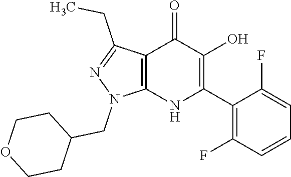 [SMILES: CCc1nn(CC2CCOCC2)c2[nH]c(-c3c(F)cccc3F)c(O)c(=O)c12] | B/Intermediate- 207 | 6-(2,6-Difluorophenyl)-3-ethyl-5-hydroxy-1- ((tetrahydro-2H-pyran-4-yl)methyl)-1H- pyrazolo[3,4-b]pyridin-4(7H)-one; ¹H NMR (400 MHz, DMSO-d₆): δ 1.22-1.39 (m, 7H), 2.04-2.10 (m, 1H), 2.88 (d, J = 6.8 Hz, 2H), 3.23 (t, J = 10.8 Hz, 2H), 3.80 (d, J = 9.2 Hz, 2H), 4.06 (br s, 2H), 7.27-7.31 (m, 2H), 7.62-7.66 (m, 1H), 8.24 (br s, 1H), 11.60 (br s, 1H); ESI (m/z) 390 (M + H)⁺. |
| 208 | 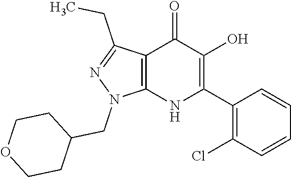 [SMILES: CCc1nn(CC2CCOCC2)c2[nH]c(-c3ccccc3Cl)c(O)c(=O)c12] | B/Intermediate- 208 | 6-(2-Chlorophenyl)-3-ethyl-5-hydroxy-1- ((tetrahydro-2H-pyran-4-yl)methyl)-1H- pyrazolo[3,4-b]pyridin-4(7H)-one; ¹H NMR (400 MHz, DMSO-d₆): δ 1.23-1.38 (m, 7H), 2.05-2.07 (m, 1H), 2.87 (q, J = 7.6 Hz, 2H), 3.22 (t, J = 10.4 Hz, 2H), 3.79-3.82 (m, 2H), 4.06 (d, J = 7.2 Hz, 2H), 7.49-7.56 (m, 3H), 7.65 (d, J = 7.6 Hz, 1H), 7.90 (s, 1H), 11.63 (s, 1H). |
| 209 | 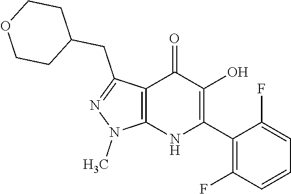 [SMILES: Cn1nc(CC2CCOCC2)c2c(=O)c(O)c(-c3c(F)cccc3F)[nH]c21] | B/Intermediate- 209 | 6-(2,6-Difluorophenyl)-5-hydroxy-1-methyl-3- ((tetrahydro-2H-pyran-4-yl)methyl)-1H- pyrazolo[3,4-b]pyridin-4(7H)-one; ¹H NMR (400 MHz, DMSO-d₆): δ 1.23-1.33 (m, 2H), 1.54-1.57 (m, 2H), 2.08-2.10 (m, 1H), 2.82 (d, J = 6.0 Hz, 2H), 3.23 (t, J = 10.0 Hz, 2H), 3.80- 3.84 (m, 5H), 7.26-7.30 (m, 2H), 7.63-7.70 (m, 1H), 8.25 (br s, 1H), 11.75 (s, 1H); ESI (m/z) 376 (M + H)⁺. |
| 210 | 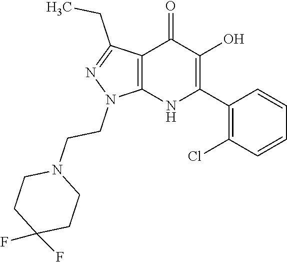 [SMILES: CCc1nn(CCN2CCC(F)(F)CC2)c2[nH]c(-c3ccccc3Cl)c(O)c(=O)c12] | B/Intermediate- 210 | 6-(2-Chlorophenyl)-1-(2-(4,4-difluoropiperidin- 1-yl)ethyl)-3-ethyl-5-hydroxy-1H-pyrazolo[3,4- b]pyridin-4(7H)-one; ¹H NMR (400 MHz, DMSO-d₆): δ 1.28 (t, J = 7.6 Hz, 3H), 1.74-1.84 (m, 4H), 2.48-2.54 (m, 2H), 2.78 (t, J = 5.6 Hz, 2H), 2.88 (q, J = 7.2 Hz, 2H), 3.32-3.38 (m, 2H), 4.27 (t, J = 5.6 Hz, 2H), 7.49-7.52 (m, 3H), 7.61- 7.65 (m, 1H), 7.91 (s, 1H), 11.80 (br s, 1H). |
| 211 | 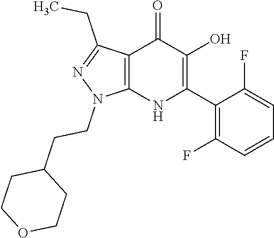 [SMILES: CCc1nn(CCC2CCOCC2)c2[nH]c(-c3c(F)cccc3F)c(O)c(=O)c12] | B/Intermediate- 211 | 6-(2,6-Difluorophenyl)-3-ethyl-5-fluoro-1-(2- (tetrahydro-2H-pyran-4-yl)ethyl)-1H- pyrazolo[3,4-b]pyridin-4(7H)-one; ¹H NMR (400 MHz, DMSO-d₆): δ 1.16-1.18 (m, 2H), 1.27 (t, J = 7.6 Hz, 3H), 1.40-1.42 (m, 1H), 1.56- 1.59 (m, 2H), 1.65-1.70 (m, 2H), 2.87 (q, J = 7.6 Hz, 2H), 3.21 (t, J = 11.6 Hz, 2H), 3.79-3.82 (m, 2H), 4.17 (t, J = 7.2 Hz, 2H), 7.29 (t, J = 8.0 Hz, 2H), 7.62-7.66 (m, 1H), 8.25 (s, 1H), 11.65 (s, 1H); ESI (m/z) 404 (M + H)⁺. |
| 212 | 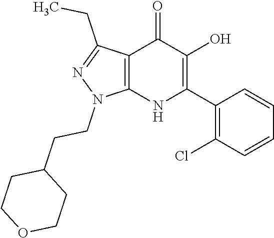 [SMILES: CCc1nn(CCC2CCOCC2)c2[nH]c(-c3ccccc3Cl)c(O)c(=O)c12] | B/Intermediate- 212 | 6-(2-Chlorophenyl)-3-ethyl-5-hydroxy-1-(2- (tetrahydro-2H-pyran-4-yl)ethyl)-1H- pyrazolo[3,4-b]pyridin-4(7H)-one; ¹H NMR (400 MHz, DMSO-d₆): δ 1.18-1.22 (m, 2H), 1.27 (t, J = 7.2 Hz, 3H), 1.41-1.43 (br s, 1H), 1.56-1.59 (m, 2H), 1.65-1.70 (m, 2H), 2.87 (q, J = 7.6 Hz, 2H), 3.16-3.34 (m, 2H), 3.78-3.82 (m, 2H), 4.18 (t, J = 7.2 Hz, 2H), 7.49-7.55 (m, 3H), 7.64 (d, J = 7.6 Hz, 1H), 7.90 (s, 1H), 11.56 (s, 1H); ESI (m/z) 402 (M + H)⁺. |
| 213 | 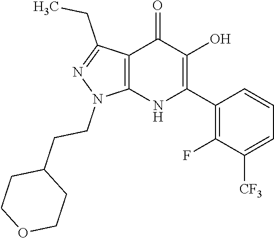 [SMILES: CCc1nn(CCC2CCOCC2)c2[nH]c(-c3cccc(C(F)(F)F)c3F)c(O)c(=O)c12] | B/Intermediate- 213 | 3-Ethyl-6-(2-fluoro-3-(trifluoromethyl)phenyl)- 5-hydroxy-1-(2-(tetrahydro-2H-pyran-4- yl)ethyl)-1H-pyrazolo[3,4-b]pyridin-4(7H)-one; ¹H NMR (400 MHz, DMSO-d₆): δ 1.16-1.19 (m, 2H), 1.27 (t, J = 7.6 Hz, 3H), 1.41-1.45 (m, 1H), 1.59-1.70 (m, 4H), 2.87-2.91 (m, 2H), 3.20-3.24 (m, 2H), 3.79-3.81 (m, 2H), 4.19 (br s, 2H), 7.56- 7.61 (m, 1H), 7.90-7.94 (m, 2H), 8.27 (s, 1H), 11.58 (br s, 1H); ESI (m/z) 454 (M + H)⁺. |
| 215 | 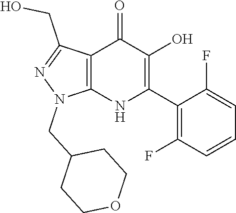 [SMILES: O=c1c(O)c(-c2c(F)cccc2F)[nH]c2c1c(CO)nn2CC1CCOCC1] | B/Intermediate- 214 | 6-(2,6-difluorophenyl)-5-hydroxy-3- (hydroxymethyl)-1-((tetrahydro-2H-pyran-4- yl)methyl)-1H-pyrazolo[3,4-b]pyridin-4(7H)- one; ¹H NMR (400 MHz, DMSO-d₆): δ 1.16- 1.39 (m, 4H), 1.99-2.09 (m, 1H), 3.226 (t, J = 10.4 Hz, 2H), 3.79-3.81 (m, 2H), 4.00-4.13 (m, 2H), 4.60-4.73 (m, 2H), 5.97-5.99 (m, 1H), 7.28 (br s, 2H), 7.63 (s, 1H), 8.62 (s, 1H), 12.06- 12.09 (br s, 1H). |
| 217 | 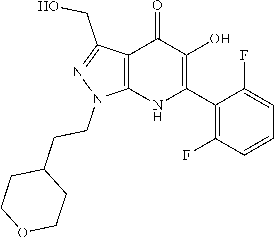 [SMILES: O=c1c(O)c(-c2c(F)cccc2F)[nH]c2c1c(CO)nn2CCC1CCOCC1] | B/Intermediate- 215 | 6-(2,6-difluorophenyl)-5-hydroxy-3- (hydroxymethyl)-1-(2-(tetrahydro-2H-pyran-4- yl)ethyl)-1H-pyrazolo[3,4-b]pyridin-4(7H)-one; ¹H NMR (400 MHz, DMSO-d₆): δ 1.08-1.21 (m, 2H), 1.37-1.38 (m, 1H), 1.60 (d, J = 12.8 Hz, 2H), 1.72 (q, J = 6.8 Hz, 2H), 3.18 (t, J = 11.6 Hz, 2H), 3.33-3.46 (m, 1H), 3.78 (dd, J₁ = 2.4 Hz, J₁ = 11.2 Hz, 2H), 4.25 (t, J = 6.8 Hz, 2H), 4.78 (s, 2H), 7.26 (t, J = 8.0 Hz, 2H), 7.58-7.62 (m, 1H), 8.67 (br s, 1H); ESI (m/z) 406 (M + H)⁺. |
| 218 | 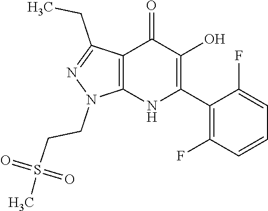 [SMILES: CCc1nn(CCS(C)(=O)=O)c2[nH]c(-c3c(F)cccc3F)c(O)c(=O)c12] | B/Intermediate- 216 | 6-(2,6-Difluorophenyl)-3-ethyl-5-hydroxy-1-(2- (methylsulfonyl)ethyl)-1H-pyrazolo[3,4- b]pyridin-4(7H)-one; ¹H NMR (400 MHz, DMSO-d₆): δ 1.29 (t, J = 7.6 Hz, 3H), 2.89-2.93 (m, 2H), 3.17 (s, 3H), 3.68 (t, J = 7.2 Hz, 2H), 4.60 (br s, 2H), 7.27-7.31 (m, 2H), 7.62-7.66 (m, 1H), 8.31 (br s, 1H), 11.58 (br s, 1H). |

### Method P:

### Example 191

### 6-(2,6-difluorophenyl)-5-hydroxy-1-methyl-3-(morpholinomethyl)-1H-pyrazolo[3,4-b]pyridin-4(7H)-one

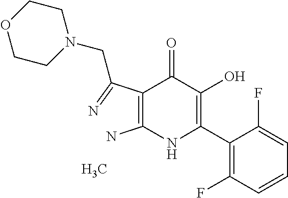

To a solution of morpholine (28.3 mg, 0.325 mmol) in dry THF (2 mL) were added (6-(2,6-Difluorophenyl)-5-hydroxy-3-(hydroxymethyl)-1-methyl-1H-pyrazolo[3,4-b]pyridin-4(7H)-one) (50 mg, 0.162 mmol) and triphenylphosphine (61.8 mg, 0.23 mmol). Then reaction was cooled at 0° C. and diisopropyl azodicarboxylate (DIAD) (46.8 μL, 0.236 mmol) was added dropwise to it.The reaction mixture was strirred at RT for 18 h.The mixute was quenched with water (2 drops) and then evapourated and purified by column to yield 18 mg of the titled product. ¹H NMR (400 MHz, DMSO-d₆): δ 2.77-2.81 (m, 4H), 3.72-3.76 (m, 4H), 3.80 (s, 3H), 4.00 (s, 2H), 7.14-7.20 (m, 2H), 7.47-7.53 (m, 1H), 8.58 (br s, 1H); ESI (m/z) 377 (M+H)⁺.

The examples 192, 195-197, 199, 201, 214, 216 & 219-224 given in the Table-2 were prepared by following either of the above mentioned procedure. The structural formulas, chemical names, ¹H NMR and MS data are provided in Table-2.

TABLE 2
Structure, chemical name, ¹H NMR and MS data of the Examples 192, 195-197, 199,
201, 214, 216 & 219-224.

| Example | | Method/ | |
|---|---|---|---|
| No | Structure | Intermediate | Chemical name, ¹H NMR and MS data |
| 192 | 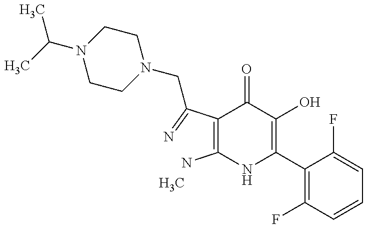 | P/ Example 188 | 6-(2,6-Difluorophenyl)-5-hydroxy-3-((4- isopropylpiperazin-1-yl)methyl)-1-methyl- 1H-pyraazolo[3,4-b]pyridin-4(7H)-one; ¹H NMR (400 MHz, DMSO-d₆): δ 0.98 (d, J = 5.6 Hz, 6H), 2.51-2.80 (m, 9H), 3.83 (s, 3H), 3.99 (s, 2H), 7.18 (t, J = 7.2 Hz, 2H), 7.48- 7.50 (m, 1H), 8.43 (br s, 1H); ESI (m/z) 418 (M + H)⁺. |
| 195 | 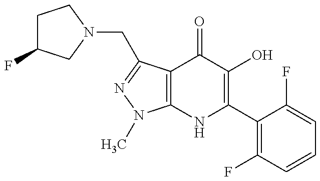 [SMILES: Cn1nc(CN2CC[C@H](F)C2)c2c(=O)c(O)c(-c3c(F)cccc3F)[nH]c21] | P/ Example 188 | (S)-6-(2,6-Difluorophenyl)-3-((3- fluoropyrrolidin-1-yl)methyl)-5-hydroxy-1- methyl-1H-pyrazolo[3,4-b]pyridin-4(7H)- one; ¹H NMR (400 MHz, DMSO-d₆): δ 2.32- 2.34 (m, 1H), 2.67-2.73 (m, 2H), 2.80-2.90 (m, 1H), 3.85 (s, 3H), 4.06 (d, J = 14.8 Hz, 2H), 4.23 (d, J = 14.4 Hz, 2H), 5.32-5.50 (m, 2H, rotamer), 7.17 (t, J = 8.0 Hz, 2H), 7.49- 7.50 (m, 1H), 8.48 (s, 1H); ESI (m/z) 379 (M + H)⁺. |
| 196 | 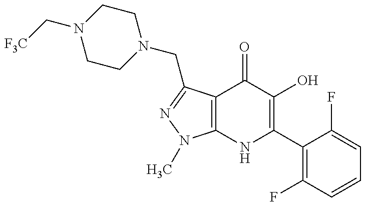 | P/ Example 188 | 3-((4-(2,2,2-trifluoroethyl)piperazin-1- yl)methyl)-6-(2,6-difluorophenyl)-5- hydroxy-1-methyl-1H-pyrazolo[3,4- b]pyridin-4(7H)-one; ¹H NMR (400 MHz, DMSO-d₆): δ 2.11-2.17 (m, 4H), 2.92 (m, 4H), 3.81 (s, 3H), 4.07 (s, 2H), 7.17 (t, J = 8.0 Hz, 2H), 7.50-7.53 (m, 1H), 8.52 (br s, 1H); ESI (m/z) 411 (M + H)⁺. |
| 197 | 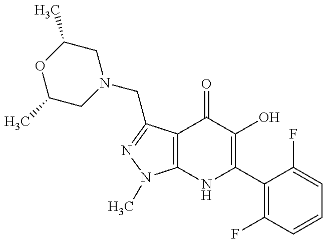 [SMILES: C[C@@H]1CN(Cc2nn(C)c3[nH]c(-c4c(F)cccc4F)c(O)c(=O)c23)C[C@H](C)O1] | P/ Example 188 | 6-(2,6-Difluorophenyl)-3-((2,6- dimethylmorpholino)methyl)-5-hydroxy-1- methyl-1H-pyrazolo[3,4-b]pyridin-4(7H)- one; ¹H NMR (400 MHz, DMSO-d₆): δ 1.08- 1.12 (m, 6H), 2.14 (t, J = 10.8 Hz, 2H), 3.05 (d, J = 10.8 Hz, 2H), 3.70-3.74 (m, 2H), 3.85 (s, 3H), 3.97 (s, 2H), 7.17 (t, J = 8.0 Hz, 2H), 7.49-7.53 (m, 1H), 8.53 (br s, 1H); ESI (m/z) 405 (M + H)⁺. |
| 199 | 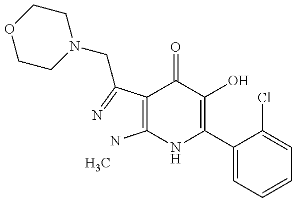 | P/ Example 198 | 6-(2-Chlorophenyl)-5-hydroxy-1-methyl-3- (morpholinomethyl)-1H-pyrazolo[3,4- b]pyridin-4(7H)-one; ¹H NMR (400 MHz, DMSO-d₆): δ 2.77 (br s, 4H), 3.74 (br s, 4H), 3.85 (s, 3H), 3.98 (s, 2H), 7.36-7.44 (m, 3H), 7.50-7.52 (m, 1H), 8.39 (br s, 1H); ESI (m/z) 375 (M + H)⁺. |
| 201 | 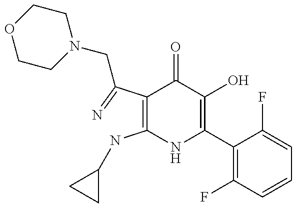 [SMILES: N=C(CN1CCOCC1)c1c(NC2CC2)[nH]c(-c2c(F)cccc2F)c(O)c1=O] | P/ Example 183 | 1-Cyclopropyl-6-(2,6-difluorophenyl)-5- hydroxy-3-(morpholinomethyl)-1H- pyrazolo[3,4-b]pyridin-4(7H)-one; ¹H NMR (400 MHz, DMSO-d₆): δ 0.97-1.14 (m, 4H), 2.76-2.82 (m, 4H), 3.69-3.73 (m, 5H), 3.97 (s, 2H), 7.17 (t, J = 7.6 Hz, 2H), 7.48-7.65 (m, 1H), 8.59 (br s, 1H). |
| 214 | 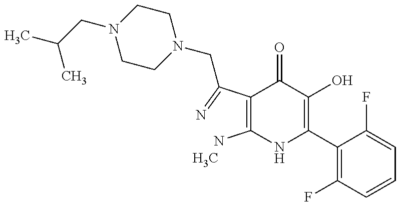 | P/ Example 188 | 6-(2,6-Difluorophenyl)-5-hydroxy-3-((4- isobutylpiperazin-1-yl)methyl)-1-methyl-1H- pyrazolo[3,4-b]pyridin-4(7H)-one; ¹H NMR (400 MHz, DMSO-d₆): δ 0.87 (d, J = 6.4 Hz, 6H), 1.75-1.78 (m, 1H), 2.08 (d, J = 7.2 Hz, 2H), 2.99-3.04 (m, 4H), 3.17-3.37 (m, 4H), 3.84 (s, 3H), 4.00 (s, 2H), 7.16 (t, J = 8.0 Hz, 2H), 7.48-7.52 (m, 2H), 8.42 (br s, 1H); ESI (m/z) 432 (M + H)⁺. |
| 216 | 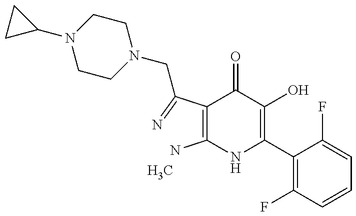 | P/ Example 188 | 3-((4-Cyclopropylpiperazin-1-yl)methyl)-6- (2,6-difluorophenyl)-5-hydroxy-1-methyl- 1H-pyrazolo[3,4-b]pyridin-4(7H)-one; ¹H NMR (400 MHz, DMSO-d₆): δ 0.33-0.45 (m, 2H), 0.85-0.86 (m, 2H), 1.65-1.69 (m, 2H), 2.51-2.75 (m, 3H), 3.23-3.84 (m, 4H), 3.89 (s, 3H), 3.99 (s, 2H), 7.18 (t, J = 7.6 Hz, 2H), 7.47-7.54 (m, 1H), 8.47 (s, 1H), 14.76 (br s, 1H); ESI (m/z) 416 (M + H)⁺. |
| 219 | 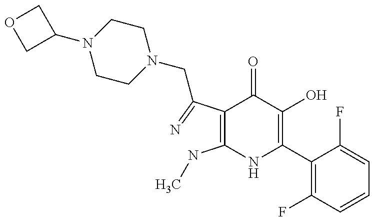 [SMILES: CNc1[nH]c(-c2c(F)cccc2F)c(O)c(=O)c1C(=N)CN1CCN(C2COC2)CC1] | P/ Example 188 | 6-(2,6-Difluorophenyl)-5-hydroxy-1-methyl- 3-((4-(oxetan-3-yl)piperazin-1-yl)methyl)- 1H-pyrazolo[3,4-b]pyridin-4(7H)-one; ¹H NMR (400 MHz, DMSO-d₆): δ 2.45-2.51 (m, 2H), 2.82-2.86 (m, 4H), 3.42-3.48 (m, 5H), 3.84 (s, 3H), 4.01 (s, 2H), 4.44 (t, J = 6.0 Hz, 2H), 4.55 (t, J = 6.4 Hz, 2H), 7.17 (t, J = 8.0 Hz, 2H), 7.48-7.52 (m, 1H), 8.48 (s, 1H). |
| 220 | 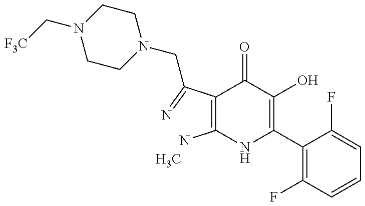 | P/ Example 188 | 6-(2,6-Difluorophenyl)-5-hydroxy-1-methyl- 3-((4-(2,2,2-trifluoroethyl)piperazin-1- yl)methyl)-1H-pyrazolo[3,4-b]pyridin- 4(7H)-one; ¹H NMR (400 MHz, DMSO-d₆): δ 3.23-3.31 (m, 9H), 3.84 (s, 4H), 4.0 (s, 2H), 7.15-7.19 (m, 2H), 7.49-7.53 (m, 1H), 8.52 (s, 1H), 14.53-14.58 (br s, 1H); ESI (m/z) 458 (M + H)⁺. |
| 221 | 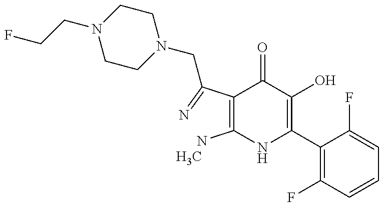 | P/ Example 188 | 6-(2,6-Difluorophenyl)-3-((4-(2- fluoroethyl)piperazin-1-yl)methyl)-5- hydroxy-1-methyl-1H-pyrazolo[3,4- b]pyridin-4(7H)-one; ¹H NMR (400 MHz, DMSO-d₆): δ 2.45-2.82 (m, 9H), 3.84 (s, 4H), 4.0 (s, 2H), 4.49 (t, J = 4.8 Hz, 1H), 4.61 (t, J = 4.4 Hz, 1H), 7.15-7.17 (m, 2H), 7.49-7.52 (m, 1H), 8.47 (s, 1H), 14.63-14.75 (br s, 1H); ESI (m/z) 422 (M + H)⁺. |
| 222 | 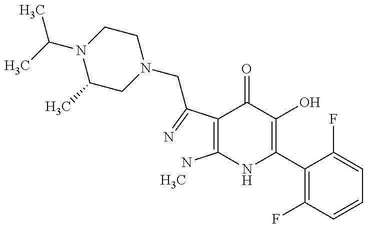 | P/ Example 188 | (S)-6-(2,6-Difluorophenyl)-5-hydroxy-3-((4- isopropyl-3-methylpiperazin-1-yl)methyl)-1- methyl-1H-pyrazolo[3,4-b]pyridin-4(7H)- one; ¹H NMR (400 MHz, DMSO-d₆): δ 0.85 (d, J = 6.4 Hz, 3H), 1.01-1.21 (m, 6H), 2.50- 3.34 (m, 7H), 3.92 (s, 3H), 3.96-4.01 (m, 2H), 4.73-4.79 (m, 1H), 7.14-7.18 (m, 2H), 7.48- 7.54 (m, 1H), 8.44 (s, 1H); ESI (m/z) 432 (M + H)⁺. |
| 223 | 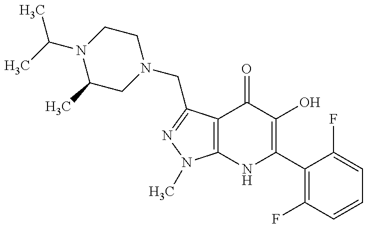 [SMILES: CC(C)N1CCN(Cc2nn(C)c3[nH]c(-c4c(F)cccc4F)c(O)c(=O)c23)C[C@H]1C] | P/ Example 188 | (R)-6-(2,6-Difluorophenyl)-5-hydroxy-3-((4- isopropyl-3-methylpiperazin-1-yl)methyl)-1- methyl-1H-pyrazolo[3,4-b]pyridin-4(7H)- one; ¹H NMR (400 MHz, DMSO-d₆): δ 0.84- 0.85 (m, 3H), 0.86-1.11 (m, 6H), 2.29-3.84 (m, 5H), 3.96 (s, 6H), 3.98-4.01 (m, 2H), 7.15-7.18 (m, 1H), 7.47-7.54 (m, 2H), 8.44 (s, 1H), 14.75-14.80 (m, 1H); ESI (m/z) 432 (M + H)⁺. |
| 224 | 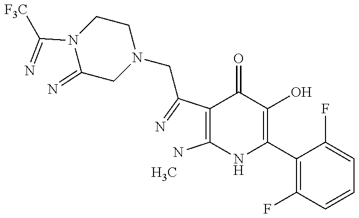 | P/ Example 188 | 6-(2,6-Difluorophenyl)-5-hydroxy-1-methyl- 3-((3-(trifluoromethyl)-5,6-dihydro- [1,2,4]triazolo[4,3-a]pyrazin-7(8H)- yl)methyl)-1H-pyrazolo[3,4-b]pyridin- 4(7H)-one; ¹H NMR (400 MHz, DMSO-d₆): δ 3.34-3.39 (br s, 4H), 3.88 (s, 3H), 4.02-4.40 (br s, 4H), 7.21-7.22 (br s, 2H), 7.54 (br s, 1H), 8.30-8.50 (m, 1H); ESI (m/z) 482 (M + H)⁺. |

### Method C:

### Example 225

### Synthesis of 5-hydroxy-6-(4-hydroxyphenyl)-1-methyl-3-(trifluoromethyl)-1,7-dihydro-4H-pyrazolo[3,4-b]pyridin-4-one

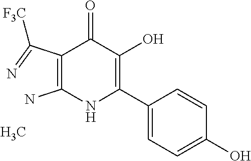

A suspension of 5-hydroxy-6-(4-methoxyphenyl)-1-methyl-3-(trifluoromethyl)-1,7-dihydro-4H-pyrazolo[3,4-b]pyridin-4-one (100 mg, 0.29 mmol) in aqueous hydrogen bromide (3.0 mL) was heated to 100° C. for 48 h. The reaction mixture was cooled to RT and quenched with saturated sodium bicarbonate solution (100 mL). The product was extracted in ethyl acetate (50 mL×3). The combined organic extracts were dried under anhydrous sodium sulfate, filtered and concentrated. The obtained product was purified by silica gel column chromatography to yield 14 mg of the product as a solid. ¹H NMR (300 MHz, DMSO-d₆): δ 4.01 (s, 3H), 6.85-6.90 (m, 2H), 7.70-7.75 (m, 2H), 8.52 (br s, 1H), 9.77 (br s, 1H); ESI (m/z) 326 (M+H)⁺.

### Method D:

### Example 226

### Synthesis of 6-(2,6-difluorophenyl)-1,3-dimethyl-4-oxo-4,7-dihydro-1H-pyrazolo[3,4-b]pyridin-5-yl methyl carbonate

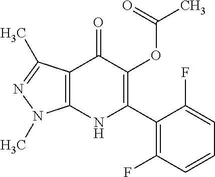 [SMILES: CC(=O)Oc1c(-c2c(F)cccc2F)[nH]c2c(c(C)nn2C)c1=O]

To a stirred solution of 6-(2,6-difluorophenyl)-5-hydroxy-1,3-dimethyl-1,7-dihydro-4H-pyrazolo[3,4-b]pyridin-4-one (25 mg, 0.08 mmol) in THF (3.0 mL) were added pyridine (8.3 μL, 0.10 mmol) and acetic anhydride (9.7 μL, 0.10 mmol) at RT. The reaction mixture was stirred overnight at RT. The reaction mixture was quenched with 1 N HCl (5.0 mL) and extracted with ethyl acetate (2×10 mL). The combined organic layers were dried over anhydrous sodium sulfate and concentrated under reduced pressure. The product obtained was purified by silica gel column chromatography to yield 22 mg of the product as a solid. ¹H NMR (300 MHz, DMSO-d₆): δ 2.05 (s, 3H), 2.46 (s, 3H), 3.97 (s, 3H), 7.26 (d, J=7.8 Hz, 2H), 7.62 (br s, 1H); APCI (m/z) 334 (M-FH)⁺.

### Method E:

### Example 227

### Synthesis of 5-(2-chlorophenyl)-6-hydroxy-2,3-dimethylpyrano[3,2-c]pyrazol-7(2H)-one

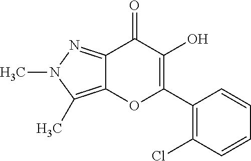 [SMILES: Cc1c2oc(-c3ccccc3Cl)c(O)c(=O)c2nn1C]

To a stirred solution of 3-(2-chlorophenyl)-1-(4-hydroxy-1,5-dimethyl-1H-pyrazol-3-yl)prop-2-en-1-one (Intermediate-59, 440 mg, 1.59 mmol) in ethanol (5.0 mL), a solution of sodium hydroxide (127 mg, 3.18 mmol) in water (1.2 mL) was added. The reaction mixture was cooled to 0° C. and was slowly added hydrogen peroxide (35%, 339 μL, 3.49 mmol) at the same temperature. The reaction mixture was stirred at RT for 18 h. The solvent was evaporated under reduced pressure and 1N HCl (20 mL) was added to the residue. The precipitate obtained was filtered and dried under vacuum to afford 190 mg of the product as a solid. ¹H NMR (300 MHz, DMSO-d₆): δ 2.39 (s, 3H), 3.98 (s, 3H), 7.48-7.57 (m, 2H), 7.62 (d, J=7.5 Hz, 2H), 8.92 (br s, 1H); APCI (m/z) 291(M+H)⁺.

The examples 228-229 were prepared by following the above mentioned procedure. The structural formulas, chemical names, ¹H NMR and MS data of the examples 228-229 are provided in Table-3.

TABLE 3
Structure, chemical name, ¹H NMR and MS data of Examples 228-229.

| Example | | Method/ | |
|---|---|---|---|
| No | Structure | Intermediate | Chemical name, ¹H NMR and MS data |
| 228 | 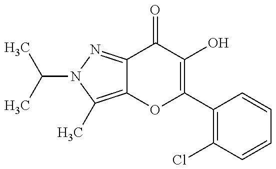 [SMILES: Cc1c2oc(-c3ccccc3Cl)c(O)c(=O)c2nn1C(C)C] | E/ Intermediate 62 | 5-(2-Chlorophenyl)-6-hydroxy-3-methyl-2-(propan- 2-yl)pyrano[3,2-c]pyrazol-7(2H)-one; ¹H NMR (300 MHz, DMSO-d₆): δ 1.47 (d, J = 6.3 Hz, 6H), 2.42 (s, 3H), 4.68-4.82 (m, 1H), 7.46-7.62 (m, 4H), 8.97 (br s, 1H); APCI (m/z) 319 (M + H)⁺. |
| 229 | 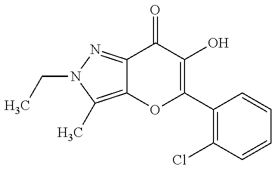 [SMILES: CCn1nc2c(=O)c(O)c(-c3ccccc3Cl)oc2c1C] | E/ Intermediate 63 | 5-(2-Chlorophenyl)-2-ethyl-6-hydroxy-3- methylpyrano[3,2-c]pyrazol-7(2H)-one; ¹H NMR (300 MHz, DMSO-d₆): δ 1.41 (t, J = 6.9 Hz, 3H), 2.41 (s, 3H), 4.30 (q, J = 6.9 Hz, 2H), 7.42-7.63 (m, 2H), 7.58-7.65 (m, 2H), 8.96 (br s, 1H); APCI (m/z) 305 (M + H)⁺. |

### Method F:

### Example 230

### Synthesis of 6-(2-chlorophenyl)-5-hydroxy-1,3-dimethylpyrano[2,3-c]pyrazol-4(1H)-one

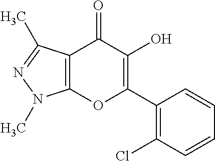 [SMILES: Cc1nn(C)c2oc(-c3ccccc3Cl)c(O)c(=O)c12]

To a stirred solution of 6-(2-chlorophenyl)-5-methoxy-1,3-dimethylpyrano[2,3-c]pyrazol-4(1H)-one (200 mg, 0.65 mmol) in dichloromethane (1.0 mL), borontribromide in dichloromethane (1M, 2.6 mL, 2.62 mmol) was added at RT. The mixture was stirred overnight at same temperature. The reaction was concentrated under reduced pressure and quenched with saturated aqueous sodium bicarbonate solution. The precipitated solid was filtered and dried well. The product obtained was purified by silica gel column chromatography to yield 118 mg of the titled product as a solid. ¹H NMR (300 MHz, DMSO-d₆): δ 2.44 (s, 3H), 3.72 (s, 3H), 7.49-7.56 (m, 2H), 7.63-7.68 (m, 2H), 9.13 (s, 1H); ESI (m/z) 291 (M+H)⁺.

The examples 231-232 were prepared by following the above mentioned procedure. The structural formulas, chemical names, ¹H NMR and MS data of the examples 231-232 are provided in Table-4.

TABLE 4
Structure, chemical name, ¹H NMR and MS data of Examples 231-232.

| Example | | Method/ | |
|---|---|---|---|
| No | Structure | Intermediate | Chemical name, ¹H NMR and MS data |
| 231 | 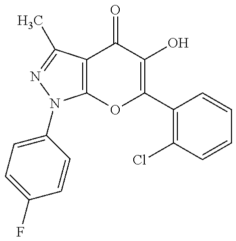 [SMILES: Cc1nn(-c2ccc(F)cc2)c2oc(-c3ccccc3Cl)c(O)c(=O)c12] | F/ Intermediate 65 | 6-(2-Chlorophenyl)-1-(4-fluorophenyl)-5-hydroxy-3- methylpyrano[2,3-c]pyrazol-4(1H)-one; ¹H NMR (300 MHz, DMSO-d₆): δ 2.55 (s, 3H), 7.43 (t, J = 8.7 Hz, 2H), 7.50-7.56 (m, 2H), 7.57-7.71 (m, 2H), 7.78-7.81 (m, 2H), 9.39 (br s, 1H); ESI (m/z) 371 (M + H)⁺. |
| 232 | 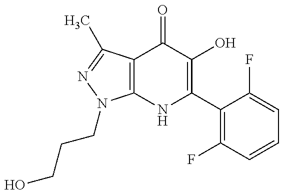 [SMILES: Cc1nn(CCCO)c2[nH]c(-c3c(F)cccc3F)c(O)c(=O)c12] | F/ Intermediate 92 | 6-(2,6-Difluorophenyl)-5-hydroxy-1-(3- hydroxypropyl)-3-methyl-1H-pyrazolo[3,4-b]pyridin- 4(7H)-one; ¹H NMR (300 MHz, DMSO-d₆): δ 1.80- 1.90 (m, 2H), 2.48 (s, 3H), 3.30-3.36 (m, 2H), 4.11-4.17 (m, 2H), 7.15-7.35 (m, 3H), 7.57-7.65 (m, 1H), 8.20 (br s, 1H), 11.64 (s, 1H); ESI (m/z) 336 (M + H)⁺. |

### Method G:

### Example 233

### Synthesis of 6-(2-chlorophenyl)-5-methoxy-1,3-dimethyl-1H-pyrazolo[3,4-b]pyridin-4(7H)-one

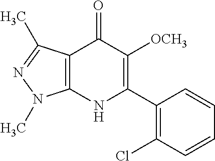 [SMILES: COc1c(-c2ccccc2Cl)[nH]c2c(c(C)nn2C)c1=O]

### Step 1: tert-Butyl 6-(2-chlorophenyl)-5-hydroxy-1,3-dimethyl-4-oxo-1,4-dihydro-7H-pyrazolo[3,4-b]pyridine-7-carboxylate

To a stirred suspension of 6-(2-chlorophenyl)-5-hydroxy-1,3-dimethyl-1,7-dihydro-4H-pyrazolo[3,4-b]pyridin-4-one (100 mg, 0.34 mmol) in THF (2.0 mL), BOC anhydride (75 mg, 0.34 mmol) followed by DMAP (5.0 mg, 0.03 mmol) was added at RT and the reaction mixture was stirred for 2 h. The solvent was evaporated under reduced pressure and the residue was purified by silica gel column chromatography to obtain 270 mg of the product as a solid. ¹H NMR (300 MHz, DMSO-d₆): δ 1.31 (s, 9H), 2.49 (s, 3H), 3.82 (s, 3H), 7.30-7.70 (m, 4H), 8.31 (s, 1H), 12.24 (br s, 1H).

### Step 2: tert-Butyl 6-(2-chlorophenyl)-5-methoxy-1,3-dimethyl-4-oxo-1,4-dihydro-7H-pyrazolo[3,4-b]pyridine-7-carboxylate

To a stirred solution of Step 1 intermediate (150 mg, 0.38 mmol) in dry DMF (1.5 mL) was added potassium carbonate (63.7 mg, 0.46 mmol) followed by methyl iodide (26.5 μL, 0.42 mmol) at RT and the resulting reaction mixture was stirred for 1 h. The mixture was acidified with 1 N citric acid till pH 2-3. The precipitated solid was filtered and dried well to obtain 112 mg of the desired product. ¹H NMR (300 MHz, CDCl₃): δ 1.29 (s, 9H), 2.64 (s, 3H), 4.03 (s, 3H), 4.19 (s, 3H), 7.26-7.52 (m, 4H).

### Step 3: 6-(2-chlorophenyl)-5-methoxy-1,3-dimethyl-1H-pyrazolo[3,4-b]pyridin-4(7H)-one

To a stirred solution of Step 2 Intermediate (135 mg, 0.33 mmol) in dichloromethane (2.0 mL), trifluoroacetic acid (123 μL, 1.67 mmol) was added at RT and the reaction mixture was stirred for 1 h. The reaction mixture was concentrated, basified with saturated aqueous NaHCO₃ solution till pH 8 and extracted with ethyl acetate (10 mL×2). The organic layer was dried over anhydrous sodium sulfate and concentrated under reduced pressure. The compound obtained was purified by silica gel column chromatography to obtain 31 mg of the desired product as a solid. ¹H NMR (300 MHz, CDCl₃): δ 2.67 (s, 3H), 4.00 (s, 3H), 4.15 (s, 3H), 5.09 (br s, 1H), 7.35-7.54 (m, 4H); ESI (m/z) 304 (M)⁺.

The examples given in the Table-5 were prepared by following the above mentioned procedure. The structural formulas, chemical names, ¹H NMR and MS data of the examples are provided in the Table-5 below.

The Example 234 was prepared by following the above mentioned procedure. The structural formulae, chemical name, ¹H NMR and MS data of the Example 234 is provided in Table-5.

TABLE 5
Structure, chemical name, ¹H NMR and MS data of Example 234.

| Example | | Method/ | |
|---|---|---|---|
| No | Structure | Intermediate | Chemical name, ¹H NMR and MS data |
| 234 | 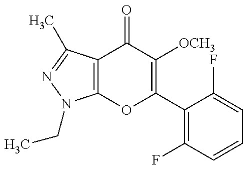 [SMILES: CCn1nc(C)c2c(=O)c(OC)c(-c3c(F)cccc3F)oc21] | G/ Example 68 | 6-(2,6-Difluorophenyl)-1-ethyl-5-methoxy-3-methyl- 1H-pyrazolo[3,4-b]pyridin-4(7H)-one; ¹H NMR (300 MHz, CDCl₃): δ 1.50 (t, J = 7.2 Hz, 3H), 2.72 (s, 3H), 4.18 (s, 3H), 4.48 (q, J = 7.2 Hz, 2H), 5.33 (br s, 1H), 7.06 (t, J = 7.8 Hz, 2H), 7.35-7.44 (m, 1H); APCI (m/z) 320 (M + H)⁺. |

### Method H:

### Example 235

### Synthesis of N-[4-(1-ethyl-5-hydroxy-3-methyl-4-oxo-4,7-dihydro-1H-pyrazolo[3,4-b]pyridin-6-yl)-3-fluorophenyl]methanesulfonamide

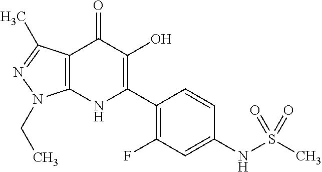 [SMILES: CCn1nc(C)c2c(=O)c(O)c(-c3ccc(NS(C)(=O)=O)cc3F)[nH]c21]

To a stirred solution of 6-(4-amino-2-fluorophenyl)-1-ethyl-5-hydroxy-3-methyl-1,7-dihydro-4H-pyrazolo[3,4-b]pyridin-4-one (110 mg, 0.36 mmol) in dichloromethane (2.0 mL), pyridine (40 μL, 0.50 mmol) and methanesulfonyl chloride (30 μL, 0.36 mmol) were added at 0° C. The recation mixture was stirred RT for overnight. The reaction mixture was concentrated under reduced pressure and the residue was diluted with water (10 mL). The aqueous mixture was acidified with 1N HCl till pH 3-4. The precipitated solid was collected by filtration and purified by silica gel column chromatography. The solid compound thus obtained was stirred in methanol, filtered and dried to yield 45 mg of the titled product. ¹H NMR (300 MHz, DMSO-d₆): δ 1.28 (t, J=7.2 Hz, 3H), 2.47 (s, 3H), 3.13 (s, 3H), 4.17 (q, J=7.2 Hz, 2H), 7.14 (d, J=9.3 Hz, 2H), 7.46-7.58 (m, 1H), 7.96 (br s, 1H), 10.27 (br s, 1H), 11.48 (s, 1H); APCI (m/z) 381 (M+H)⁺.

The Examples 236-237 were prepared by following the above mentioned procedure. The structural formulas, chemical names, ¹H NMR and MS data of the Examples 236-237 are provided in Table-6.

Table-6: Structure, chemical name, ¹H NMR and MS data of Examples 236-237.

TABLE 6
Structure, chemical name, ¹H NMR and MS data of Examples 236-237.

| Example | | | |
|---|---|---|---|
| No | Structure | Method | Chemical name, ¹H NMR and MS data |
| 236 | 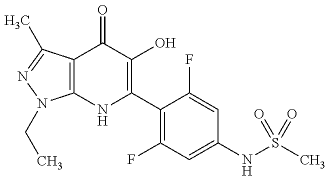 [SMILES: CCn1nc(C)c2c(=O)c(O)c(-c3c(F)cc(NS(C)(=O)=O)cc3F)[nH]c21] | H/ Intermediate- 125 | N-[4-(1-Ethyl-5-hydroxy-3-methyl-4-oxo-4,7-dihydro- 1H-pyrazolo[3,4-b]pyridin-6-yl)-3,5- difluorophenyl]methanesulfonamide; ¹H NMR (300 MHz, DMSO-d₆): δ 1.27 (t, J = 7.2 Hz, 3H), 2.46 (s, 3H), 3.20 (s, 3H), 4.14 (q, J = 7.2 Hz, 2H), 7.00 (d, J = 9.0 Hz, 2H), 8.23 (s, 1H), 10.55 (s, 1H), 11.61 (s, 1H); APCI (m/z) 399 (M + H)⁺. |
| 237 | 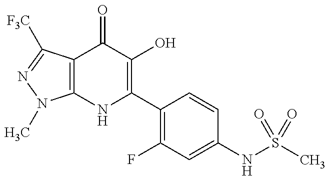 [SMILES: Cn1nc(C(F)(F)F)c2c(=O)c(O)c(-c3ccc(NS(C)(=O)=O)cc3F)[nH]c21] | H/ Intermediate- 126 | N-{3-Fluoro-4-[5-hydroxy-1-methyl-4-oxo-3- (trifluoromethyl)-4,7-dihydro-1H-pyrazolo[3,4- b]pyridin-6-yl]phenyl}methanesulfonamide; ¹H NMR (300 MHz, DMSO-d₆): δ 3.11 (s, 3H), 3.96 (s, 3H), 7.05- 7.15 (m, 2H), 7.50 (t, J = 7.5 Hz, 1H), 8.29 (br s, 1H), 10.22 (br s, 1H), 12.08 (br s, 1H); APCI (m/z) 419 (M − H)⁻. |

### Method I:

### Example 238

Synthesis of 6-(2-Chlorophenyl)-3-ethyl-5-hydroxy-1-(2-morpholinoethyl)-1H-pyrazolo[3,4-b]pyridin-4(7H)-one hydrochloride

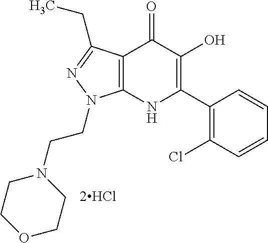 [SMILES: CCc1nn(CCN2CCOCC2)c2[nH]c(-c3ccccc3Cl)c(O)c(=O)c12]

To a stirred solution of solution of 6-(2-Chlorophenyl)-3-ethyl-5-hydroxy-1-(2-morpholinoethyl)-1H-pyrazolo[3,4-b]pyridin-4(7H)-one (100 mg, 0.248 mmol) in dry ethyl acetate (0.5 ml) was added saturated solution of dry hydrochloric acid in ethyl acetate (3 ml) and the resulting suspension was stirred for overnight. The solid obtained was collected by filtration and was stirred with dry diisopropyl ether, filtered and dried to yield 90 mg of the desired product. ¹H NMR (300 MHz, DMSO-d₆): δ 1.29 (t, J=7.5 Hz, 3H), 2.92 (q, J=7.8 Hz, 2H), 3.00-3.20 (m, 2H), 3.40-3.56 (m, 4H), 3.60-4.00 (m, 4H), 4.60-4.68 (m, 2H), 7.40-7.55 (m, 3H), 7.56-7.64 (m, 1H), 11.13 (br s, 1H); ESI (m/z) 403 (M)⁺.

The Examples 239-242 were prepared by following the above mentioned procedure. The structural formulas, chemical names, ¹H NMR and MS data of the Examples 239-242 are provided in Table-7.

TABLE 7
Structure, chemical name, ¹H NMR and MS data of Examples 239-242.

| Example | | Method/ | |
|---|---|---|---|
| No | Structure | Intermediate | Chemical name, ¹H NMR and MS data |
| 239 | 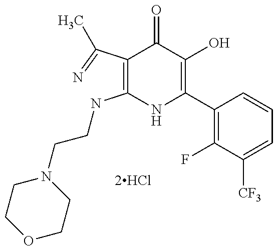 [SMILES: CC(=N)c1c(NCCN2CCOCC2)[nH]c(-c2cccc(C(F)(F)F)c2F)c(O)c1=O] | I/ Example 153 | 6-(2-Fluoro-3-(trifluoromethyl)phenyl)-5-hydroxy-3- methyl-1-(2-morpholinoethyl)-1H-pyrazolo[3,4- b]pyridin-4(7H)-one dihydrochloride; ¹H NMR (300 MHz, DMSO-d₆): δ 2.55 (s, 3H), 3.00-3.20 (m, 2H), 3.49-3.61 (m, 4H), 3.67-3.80 (m, 2H), 3.90-3.96 (m, 2H), 4.70 (t, J = 6.4 Hz, 2H), 7.54 (t, J = 7.8 Hz, 1H), 7.84-7.96 (m, 2H), 9.31 (br s, 3H), 11.05 (br s, 1H); ESI (m/z) 441 (M + H − 2HCl)⁺. |
| 240 | 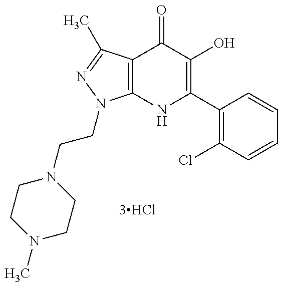 | I/ Example 155 | 6-(2-Chlorophenyl)-5-hydroxy-3-methyl-1-(2-(4- methylpiperazin-1-yl)ethyl)-1H-pyrazolo[3,4- b]pyridin-4(7H)-one trihydrochloride; ¹H NMR (500 MHz, DMSO-d₆): δ 2.52 (s, 3H), 2.78 (s, 3H), 3.18- 3.28 (m, 4H), 3.40-3.47 (m, 2H), 3.48-3.61 (m, 4H), 4.57 (t, J = 6.0 Hz, 2H), 7.45-7.53 (m, 3H), 7.58-7.62 (m, 1H), 11.43 (br s, 1H). ESI (m/z) 440 (M)⁺. |
| 241 | 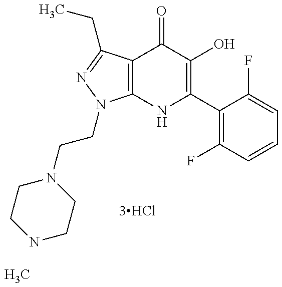 | I/ Example 166 | 6-(2,6-Difluorophenyl)-3-ethyl-5-hydroxy-1-(2-(4- methylpiperazin-1-yl)ethyl)-1H-pyrazolo[3,4- b]pyridin-4(7H)-one trihydrochloride; ¹H NMR (400 MHz, DMSO-d₆): δ 1.31 (t, J = 7.2 Hz, 3H), 2.81 (s, 3H), 2.91 (q, J = 7.2 Hz, 2H), 3.27-4.02 (m, 10H), 4.67 (t, J = 4.4 Hz, 2H), 7.21-7.28 (m, 2H), 7.57-7.62 (m, 1H), 11.83-11.95 (m, 1H).; ESI (m/z) 440 (M)⁺. |
| 242 | 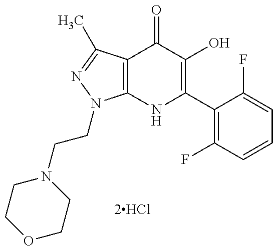 | I/ Example 156 | 6-(2,6-Difluorophenyl)-3-ethyl-5-hydroxy-1-(2- morpholinoethyl)-1H-pyrazolo[3,4-b]pyridin-4(7H)- one dihydrochloride; ¹H NMR (400 MHz, DMSO-d₆): δ 1.31 (t, J = 7.6 Hz, 3H), 2.93 (q, J = 7.6 Hz, 2H), 3.10-3.17 (m, 2H), 3.50-3.95 (m, 6H), 4.62-4.72 (m, 2H), 4.64-4.73 (m, 2H), 7.24 (t, J = 7.6 Hz, 2H), 7.59 (t, J = 7.2 Hz, 1H), 10.82-10.99 (m, 1H); APCI (m/z) 406 (M + H − 2HCl)⁺. |

### Method J:

### Example 243

### Synthesis of 6-(2-Chlorophenyl)-5-hydroxy-3-methyl-1-(2-(4-methylpiperazin-1-yl)ethyl)-1H-pyrazolo[3,4-b]pyridin-4(7H)-one fumarate

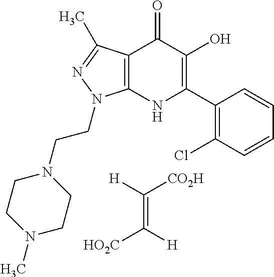

To a stirred solution of solution of 6-(2-chlorophenyl)-5-hydroxy-3-methyl-1-(2-(4-methylpiperazin-1-yl)ethyl)-1H-pyrazolo[3,4-b]pyridin-4(7H)-one (100 mg, 0.249 mmol) in acetone (2.0 ml), fumaric acid (28.8 mg, 0.240 mmol) was added and the resulting suspension was stirred for overnight. The solid obtained was collected by filtration, washed with acetone and dried to yield 125 mg of the desied product. ¹H NMR (500 MHz, DMSO-d₆): δ 2.14 (s, 3H), 2.20-2.29 (m, 2H), 2.48 (s, 3H), 2.40-2.57 (m, 6H), 2.72 (t, J=4.8 Hz, 2H), 4.28 (t, J=6.0 Hz, 2H), 6.58 (s, 2H), 7.45-7.54 (m, 3H), 7.62 (d, J=7.5 Hz, 1H).

The Examples 244-245 were prepared by following the above mentioned procedure. The structural formulas, chemical names, ¹H NMR and MS data of the Examples 244-245 are provided in Table-8.

TABLE 8
Structure, chemical name, ¹H NMR and MS data of Examples 244-245.

| Example | | Method/ | |
|---|---|---|---|
| No | Structure | Intermediate | Chemical name, ¹H NMR and MS data |
| 244 | 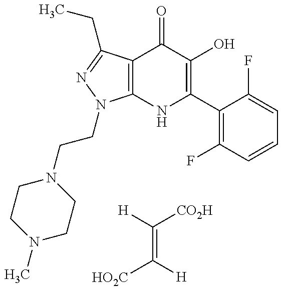 [SMILES: CCc1nn(CCN2CCN(C)CC2)c2[nH]c(-c3c(F)cccc3F)c(O)c(=O)c12 ; O=C(O)/C=C/C(=O)O] | J/ Example 166 | 6-(2,6-Difluorophenyl)-3-ethyl-5-hydroxy-1-(2-(4- methylpiperazin-1-yl)ethyl)-1H-pyrazolo[3,4- b]pyridin-4(7H)-one fumarate; ¹H NMR (400 MHz, DMSO-d₆): ¹H NMR (400 MHz, DMSO-d₆): δ 1.28 (t, J = 7.2 Hz, 3H), 2.21 (s, 3H), 2.26-2.54 (m, 8H), 2.74 (t, J = 6.8 Hz, 2H), 2.90 (q, J = 7.6 Hz, 2H), 4.29 (t, J = 6.4 Hz, 2H), 6.58 (s, 2H), 7.26 (t, J = 8.0 Hz, 2H), 7.54-7.64 (m, 1H). |
| 245 | 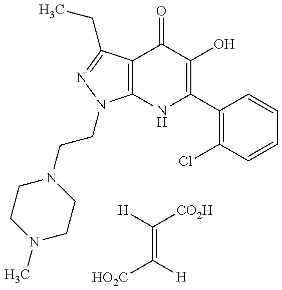 [SMILES: CCc1nn(CCN2CCN(C)CC2)c2[nH]c(-c3ccccc3Cl)c(O)c(=O)c12 ; O=C(O)/C=C/C(=O)O] | J/ Example 155 | 6-(2-Chlorophenyl)-3-ethyl-5-hydroxy-1-(2-(4- methylpiperazin-1-yl)ethyl)-1H-pyrazolo[3,4- b]pyridin-4(7H)-one fumarate; ¹H NMR (400 MHz, DMSO-d₆): δ 1.28 (t, J = 7.2 Hz, 3H), 2.08-2.52 (m, 11H), 2.74 (t, J = 6.4 Hz, 2H), 2.86 (q, J = 7.6 Hz, 2H), 4.30 (t, J = 6.0 Hz, 2H), 6.59 (s, 2H), 7.47-7.55 (m, 3H), 7.63 (d, J = 7.6 Hz, 1H), 12.20-12.60 (m, 2H). |

### Method K:

### Example 246

### Synthesis of 6-(2-Chlorophenyl)-5-hydroxy-3-methyl-1-(2-(4-methylpiperazin-1-yl)ethyl)-1H-pyrazolo[3,4-b]pyridin-4(7H)-one hemi fumarate

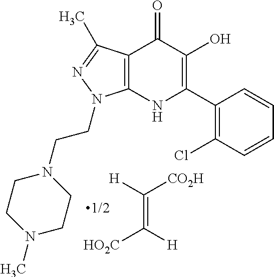

To a stirred solution of solution of 6-(2-chlorophenyl)-5-hydroxy-3-methyl-1-(2-(4-methylpiperazin-1-yl)ethyl)-1H-pyrazolo[3,4-b]pyridin-4(7H)-one (150 mg, 0.374 mmol) in acetone (4.0 ml), fumaric acid (26 mg, 0.224 mmol) was added and the resulting suspension was stirred for overnight. The solid obtained was collected by filtration, washed with acetone and dried well to yield 153 mg of the titled product. ¹H NMR (400 MHz, DMSO-d₆): δ 2.12 (s, 3H), 2.21-2.30 (m, 2H), 2.48 (s, 3H), 2.39-2.58 (m, 6H), 2.73 (t, J=6.0 Hz, 2H), 4.28 (t, J=6.0 Hz, 2H), 6.57 (s, 1H), 7.47-7.54 (m, 3H), 7.62 (d, J=8.0 Hz, 1H).

The Examples 247-248 were prepared by following the above mentioned procedure. The structural formulas, chemical names, ¹H NMR and MS data of the Examples 247-248 are provided in Table-9.

TABLE 9
Structure, chemical name, ¹H NMR and MS data of Examples 247-248.

| Example | | Method/ | |
|---|---|---|---|
| No | Structure | Intermediate | Chemical name, ¹H NMR and MS data |
| 247 | 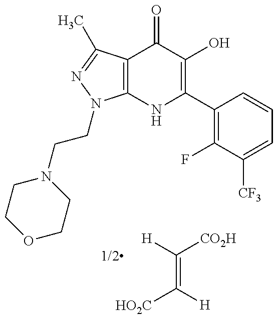 [SMILES: Cc1nn(CCN2CCOCC2)c2[nH]c(-c3cccc(C(F)(F)F)c3F)c(O)c(=O)c12 ; O=C(O)/C=C/C(=O)O] | K/ Example 153 | 6-(2-Fluoro-3-(trifluoromethyl)phenyl)-5-hydroxy-3- methyl-1-(2-morpholinoethyl)-1H-pyrazolo[3,4- b]pyridin-4(7H)-one hemi fumarate; ¹H NMR (400 MHz, DMSO-d₆): δ 2.44 (s, 3H), 2.45-2.52 (m, 4H), 2.73 (t, J = 6.4 Hz, 2H), 3.47 (t, J = 4.4 Hz, 4H), 4.30 (t, J = 6.4 Hz, 2H), 6.62 (s, 1H), 7.55-7.58 (m, 1H), 7.90 (t, J = 7.6 Hz, 2H). |
| 248 | 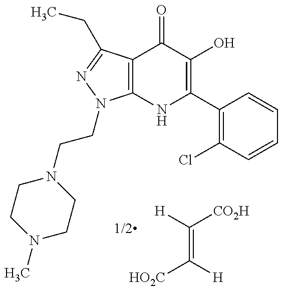 [SMILES: CCc1nn(CCN2CCN(C)CC2)c2[nH]c(-c3ccccc3Cl)c(O)c(=O)c12 ; O=C(O)/C=C/C(=O)O] | K/ Example 165 | 6-(2-Chlorophenyl)-3-ethyl-5-hydroxy-1-(2-(4- methylpiperazin-1-yl)ethyl)-1H-pyrazolo[3,4- b]pyridin-4(7H)-one hemi fumarate; ¹H NMR (400 MHz, DMSO-d₆): δ 1.30 (t, J = 7.2 Hz, 3H), 2.08-2.28 (m, 7H), 2.45-2.54 (m, 4H), 2.74 (t, J = 6.0 Hz, 2H), 2.86 (q, 7.2 Hz, 2H), 4.30 (t, J = 6.0 Hz, 2H), 6.57 (s, 1H), 7.47-7.55 (m, 3H), 7.63 (d, J = 8.0 Hz, 1H), 12.05- 12.59 (m, 2H) |

### Method L:

### Example 249

### Syntheis of 6-(2-Chlorophenyl)-5-hydroxy-3-methyl-1-(2-(4-methylpiperazin-1-yl)ethyl)-1H-pyrazolo[3,4-b]pyridin-4(7H)-one trimethanesulfonate

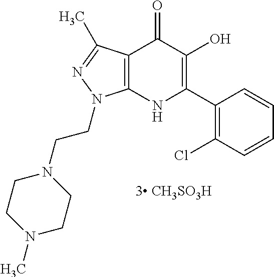 [SMILES: Cc1nn(CCN2CCN(C)CC2)c2[nH]c(-c3ccccc3Cl)c(O)c(=O)c12]

To a stirred solution of 6-(2-chlorophenyl)-5-hydroxy-3-methyl-1-(2-(4-methylpiperazin-1-yl)ethyl)-1H-pyrazolo[3,4-b]pyridin-4(7H)-one (300 mg, 0.748 mmol) in acetone (4 ml), methanesulfonic acid (160 μL, 2.40 mmol) was added at 0° C. and the resulting suspension was stiired for overnight. The solid obtained was collected by filtration, washed with acetone and dried to yield 440 mg of the desired product. ¹H NMR (500 MHz, DMSO-d₆): δ 2.38 (s, 9H), 2.53 (s, 3H), 2.86 (s, 3H), 3.12-3.30 (m, 4H), 3.47-3.54 (m, 2H), 3.64-3.74 (m, 4H), 4.54 (t, J=6.0 Hz, 2H), 7.45-7.54 (m, 3H), 7.60 (d, J=8.0 Hz, 1H), 10.08 (br s, 1H).

The Examples 250-251 were prepared by following the above mentioned procedure. The structural formulas, chemical names, ¹H NMR and MS data of the Examples 250-251 are provided in Table-10.

TABLE 10
Structure, chemical name, ¹H NMR and MS data of Examples 250-251.

| Example | | Method/ | |
|---|---|---|---|
| No | Structure | Intermediate | Chemical name, ¹H NMR and MS data |
| 250 | 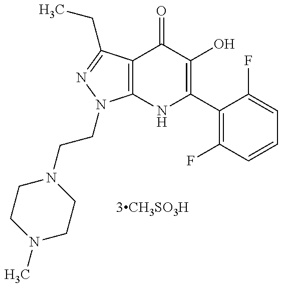 [SMILES: CCc1nn(CCN2CCN(C)CC2)c2[nH]c(-c3c(F)cccc3F)c(O)c(=O)c12] | L/ Example 166 | 6-(2,6-Difluorophenyl)-3-ethyl-5-hydroxy-1-(2-(4- methylpiperazin-1-yl)ethyl)-1H-pyrazolo[3,4-b]pyridin- 4(7H)-one trimethanesulfonate; ¹H NMR (400 MHz, DMSO-d₆): δ 1.31 (t, J = 7.2 Hz, 3H), 2.38 (s, 9H), 2.93 (q, J = 7.2 Hz, 2H), 2.90-4.07 (m, 10H), 4.50-4.56 (m, 2H), 7.26 (t, J = 7.6 Hz, 2H), 7.61 (t, J = 8.0 Hz, 1H), 9.96 (br s, 1H). |
| 251 | 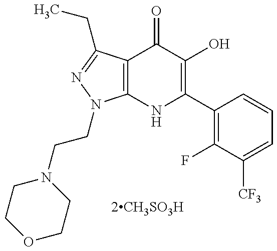 [SMILES: CCc1nn(CCN2CCOCC2)c2[nH]c(-c3cccc(C(F)(F)F)c3F)c(O)c(=O)c12] | L/ Example 164 | 3-Ethyl-6-(2-fluoro-3-(trifluoromethyl)phenyl)-5- hydroxy-1-(2-morpholinoethyl)-1H-pyrazolo[3,4- b]pyridin-4(7H)-one trimethanesulfonate; ¹H NMR (400 MHz, DMSO-d₆): δ 2.09 (s, 3H), 2.56 (s, 6H), 3.60-3.65 (m, 4H), 3.90-3.98 (m, 2H), 4.62 (t, J = 6.4 Hz, 2H), 5.05-5.49 (m, 4H), 7.56 (t, J = 7.6 Hz, 1H), 7.84-7.92 (m, 2H), 9.72 (br.s, 1H). |

### Method M:

### Example 252

### Synthesis of 6-(2-Chlorophenyl)-5-hydroxy-3-methyl-1-(2-(4-methylpiperazin-1-yl)ethyl)-1H-pyrazolo[3,4-b]pyridin-4(7H)-one hemi 2-hydroxypropane-1,2,3-tricarboxylate

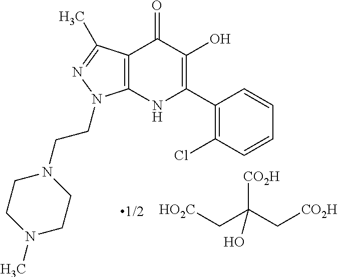

A solution of citric acid (72 mg, 0.370 mmol) in water (0.5 mL) was added to acetone (3 ml) at RT. A suspension of 6-(2-chlorophenyl)-5-hydroxy-3-methyl-1-(2-(4-methylpiperazin-1-yl)ethyl)-1H-pyrazolo[3,4-b]pyridin-4(7H)-one (225 mg, 0.560 mmol) in acetone (3 ml) was added to above solution and the resulting suspension was stiired for overnight. The solid obtained was collected by filtration, washed with acetone and dried to yield 230 mg of the desired product. ¹H NMR (500 MHz, DMSO-d₆): δ 2.59 (s, 3H), 2.61-3.50 (m, 13H), 2.96 (t, J=4.4 Hz, 2H), 4.36 (t, J=4.4 Hz, 2H), 7.51-7.62 (m, 3H), 7.68 (d, J=8.0 Hz, 1H).

The Examples 253-254 were prepared by following the above mentioned procedure. The structural formulas, chemical names, ¹H NMR and MS data of the Examples 253-254 are provided in Table-11.

TABLE 11
Structure, chemical name, ¹H NMR and MS data of Examples 253-254.

| Example | | Method/ | |
|---|---|---|---|
| No | Structure | Intermediate | Chemical name, ¹H NMR and MS data |
| 253 | 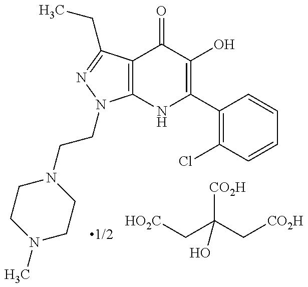 | M/ Example 165 | 6-(2-Chlorophenyl)-3-ethyl-5-hydroxy-1-(2-(4- methylpiperazin-1-yl)ethyl)-1H-pyrazolo[3,4- b]pyridin-4(7H)-one hemi 2-hydroxypropane-1,2,3- tricarboxylate; ¹H NMR (400 MHz, DMSO-d₆): δ 1.28 (t, J = 7.2 Hz, 2H), 2.33 (s, 3H), 2.49-2.68 (m, 10H), 2.76 (t, J = 6.0 Hz, 2H), 3.37 (q, J = 7.2 Hz, 2H), 4.30 (t, J = 6.0 Hz, 2H), 7.48-7.54 (m, 3H), 7.62 (d, J = 8.0 Hz, 1H), 7.85-7.95 (m, 1H), 11.02-11.52 (m, 2H). |
| 254 | 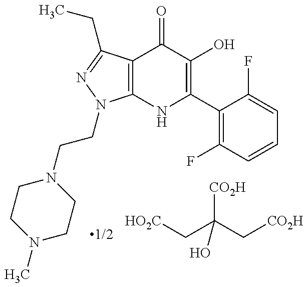 | M/ Example 166 | 6-(2,6-Difluorophenyl)-3-ethyl-5-hydroxy-1-(2-(4- methylpiperazin-1-yl)ethyl)-1H-pyrazolo[3,4- b]pyridin-4(7H)-one hemi 2-hydroxypropane-1,2,3- tricarboxylate; ¹H NMR (400 MHz, DMSO-d₆): δ 1.28 (t, J = 7.6 Hz, 3H), 2.38 (s, 3H), 2.46-2.64 (m, 10H), 2.76 (t, J = 6.6 Hz, 2.87 (q, J = 7.2 Hz, 2H), 4.29 (t, J = 6.4 Hz, 2H), 7.28 (t, J = 7.6 Hz, 2H), 7.62 (t, J = 6.8 Hz, 1H), 8.13-8.36 (m, 1H). |

### Method N:

### Example 255

### Synthesis of 6-(2-Chlorophenyl)-5-hydroxy-3-methyl-1-(2-(4-methylpiperazin-1-yl)ethyl)-1H-pyrazolo[3,4-b]pyridin-4(7H)-one 2-hydroxypropane-1,2,3-tricarboxylate

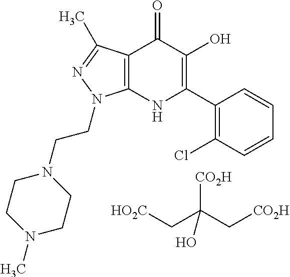

A solution of citric acid (48 mg, 0.249 mmol) in water (0.25 mL) was added to acetone (2 ml) at RT. A suspension of 6-(2-chlorophenyl)-5-hydroxy-3-methyl-1-(2-(4-methylpiperazin-1-yl)ethyl)-1H-pyrazolo[3,4-b]pyridin-4(7H)-one (100 mg, 0.560 mmol) in acetone (2 mL) was added to above solution and the resulting suspension was stiired for overnight. The solid obtained was collected by filtration, washed with acetone and dried well to yield 129 mg of the desired product. ¹H NMR (400 MHz, DMSO-d₆): δ 2.43 (s, 3H), 2.48-2.65 (m, 15H), 2.76 (t, J=4.4 Hz, 2H), 4.28 (t, J=4.4 Hz, 2H), 7.47-7.53 (m, 3H), 7.63 (d, J=7.6 Hz, 1H), 10.72-10.96 (m, 1H).

The Examples 256-257 were prepared by following the above mentioned procedure. The structural formulas, chemical names, ¹H NMR and MS data of the Examples 256-257 are provided in Table-12.

TABLE 12
Structure, chemical name, ¹H NMR and MS data of Examples 256-257.

| Example | | Method/ | |
|---|---|---|---|
| No | Structure | Intermediate | Chemical name, ¹H NMR and MS data |
| 256 | 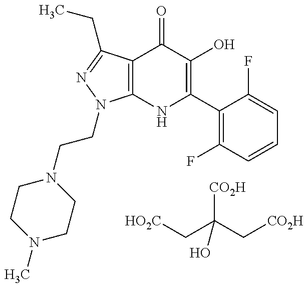 | N/ Example 166 | 6-(2,6-difluorophenyl)-3-ethyl-5-hydroxy-1-(2-(4- methylpiperazin-1-yl)ethyl)-1H-pyrazolo[3,4- b]pyridin-4(7H)-one 2-hydroxypropane-1,2,3- tricarboxylate; ¹H NMR (400 MHz, DMSO-d₆): δ 1.28 (t, J = 7.2 Hz, 3H), 2.42-2.65 (m, 17H), 3.28- 3.40 (m, 2H), 4.29 (t, J = 6.6 Hz, 2H), 7.25-7.30 (m, 2H), 7.61-7.65 (m, 1H), 10.81-11.07 (m, 3H). |
| 257 | 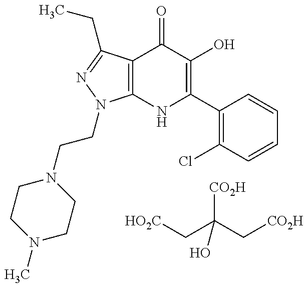 | N/ Example 165 | 6-(2-Chlorophenyl)-3-ethyl-5-hydroxy-1-(2-(4- methylpiperazin-1-yl)ethyl)-1H-pyrazolo[3,4- b]pyridin-4(7H)-one 2-hydroxypropane-1,2,3- tricarboxylate; ¹H NMR (400 MHz, DMSO-d₆): δ 1.30 (t, J = 7.2 Hz, 3H), 2.08-2.65 (m, 15H), 2.77 (t, J = 6.0 Hz, 2H), 2.88 (q, J = 7.6 Hz, 2H), 4.29 (t, J = 6.4 Hz, 2H), 7.47-7.54 (m, 2H), 7.63 (d, J = 8.0 Hz, 1H), 7.90-7.93 (m, 1H), 10.73-11.02 (m, 5H). |

### Pharmacological Activity

The inhibition of NOX4 activity was measured as inhibition of formation of reactive oxygen species (ROS) from oxygen in cell free assays. Due to the instability and reactive nature of superoxide, the read-out techniques involve detection of H₂O₂, which is a more stable product. A cell free membrane based in vitro assay was developed by preparing membranes from cells stably overexpressing human NOX4. hNOX4/HEK stable cell line was generated in-house for this purpose. Membranes from stably transfected cells overexpressing human NOX4 were prepared by homogenizing the cells in buffer (20 mM HEPES, 1 mM EGTA, 0.5 mM DTT, 0.5% protease inhibitor cocktail, 1 mM PMSF, pH 7.6), followed by centrifugation at 160000 g for 30 min. Membrane fraction (pellet) was resuspended in storage buffer (homogenization buffer with 10% glycerol and 100 mM NaCl) and stored at −80° C. Protein concentration was determined by Bradford reagent. H202 production by membranes expressing human NOX4 was measured using Amplex Red (Invitrogen) by following a slightly modified version of the manufacturer's instruction manual. Briefly, membranes were incubated in assay buffer (25 mM HEPES, 0.12 M NaCl, 3 mM KCl, 1 mM MgCl₂, pH 7.4) with Horse Radish Peroxidase (HRP) and Amplex Red. Reaction was started by addition of NADPH oxidase to the membrane mix. Antagonism of NOX4 inhibitors was measured by incubating the membrane with increasing concentrations of the inhibitors for 20 min on a plate shaker prior to addition of NADPH. H₂O₂ levels were measured for 10 min in a BMG Fluostar microplate reader with excitation and emission wavelengths of 544 nm and 590 nm respectively. Concentration response curves were plotted as a % of maximal response obtained in the absence of the inhibitor. IC₅₀ value was calculated from concentration response curve by nonlinear regression analysis using GraphPad PRISM software.

The compounds prepared were tested using the above assay procedure and the results obtained are given in Table-13. Percentage inhibition at concentrations of 1.0 μM and 10.0 μM are given in the table along with IC₅₀ (nM) details for selected examples. The compounds prepared were tested using the above assay procedure and were found to have IC₅₀ less than 1100 nM, preferably less than 100 nM, more preferably less than 50 nM.

The IC₅₀ (nM) values of some of the compounds are set forth in Table-13wherein “A” refers to an IC₅₀ value of less than 50 nM, “B” refers to IC₅₀ value in range of 50.01 to 100.0 nM, “C” refers to IC₅₀ value in range of 100.01 to 150.0 nM,and “D” refers to IC₅₀ values more than 150 nM.

TABLE 13

| | | Percentage inhibition at | | |
|---|---|---|---|---|
| S. N. | Example Number | 1.0 μM | 10.0 μM | IC₅₀ |
| 1. | Example 1 | 94.14 | 89.72 | A |
| 2. | Example 2 | 83.49 | 81.48 | C |
| 3. | Example 3 | 93.67 | 100.00 | C |
| 4. | Example 4 | 94.52 | 98.54 | D |
| 5. | Example 5 | 74.02 | 84.15 | D |
| 6. | Example 6 | 96.31 | 94.31 | C |
| 7. | Example 7 | 99.96 | 100.00 | B |
| 8. | Example 8 | 87.50 | 100.00 | B |
| 9. | Example 9 | 81.72 | 97.42 | D |
| 10. | Example 10 | 100.00 | 100.00 | A |
| 11. | Example 11 | 76.62 | 76.48 | C |
| 12. | Example 12 | 94.95 | 86.00 | B |
| 13. | Example 13 | 99.60 | 98.65 | A |
| 14. | Example 14 | 100.00 | 82.45 | B |
| 15. | Example 15 | 50.09 | — | — |
| 16. | Example 16 | 99.70 | — | D |
| 17. | Example 17 | 63.92 | — | — |
| 18. | Example 18 | 78.56 | 78.44 | C |
| 19. | Example 19 | 95.30 | 75.19 | B |
| 20. | Example 20 | 95.86 | 96.12 | A |
| 21. | Example 21 | 100.00 | 100.00 | D |
| 22. | Example 22 | 100.00 | 80.93 | D |
| 23. | Example 23 | 100.00 | 99.34 | B |
| 24. | Example 24 | 88.94 | — | D |
| 25. | Example 25 | 90.70 | 78.32 | — |
| 26. | Example 26 | 89.73 | 64.46 | B |
| 27. | Example 27 | 98.24 | 100.00 | C |
| 28. | Example 28 | 82.13 | 58.91 | C |
| 29. | Example 29 | 89.44 | 95.63 | C |
| 30. | Example 30 | 64.11 | 77.27 | D |
| 31. | Example 31 | 100.00 | 100.00 | A |
| 32. | Example 32 | 100.00 | 100.00 | A |
| 33. | Example 33 | 100.00 | 100.00 | A |
| 34. | Example 34 | 100.00 | 100.00 | A |
| 35. | Example 35 | 100.00 | 100.00 | A |
| 36. | Example 36 | 84.19 | 100.00 | D |
| 37. | Example 37 | 100.00 | 100.00 | B |
| 38. | Example 38 | 87.46 | 86.08 | C |
| 39. | Example 39 | 85.05 | 87.30 | C |
| 40. | Example 40 | 93.34 | 100.00 | A |
| 41. | Example 41 | 77.69 | 87.63 | D |
| 42. | Example 42 | 86.83 | 96.50 | C |
| 43. | Example 43 | 90.63 | 97.92 | C |
| 44. | Example 44 | 84.56 | 100.00 | D |
| 45. | Example 45 | 89.14 | 98.57 | C |
| 46. | Example 46 | 73.62 | 100.00 | C |
| 47. | Example 47 | 100.00 | 100.00 | B |
| 48. | Example 48 | 84.19 | 98.34 | C |
| 49. | Example 49 | 100.00 | 100.00 | D |
| 50. | Example 50 | 89.26 | 99.55 | C |
| 51. | Example 51 | 74.36 | 86.53 | D |
| 52. | Example 52 | 75.85 | 94.88 | D |
| 53. | Example 53 | 90.48 | 84.59 | C |
| 54. | Example 54 | 92.14 | 94.55 | C |
| 55. | Example 55 | 83.75 | 94.23 | D |
| 56. | Example 56 | 82.40 | 69.82 | A |
| 57. | Example 57 | 21.85 | 13.53 | — |
| 58. | Example 58 | 36.74 | 51.21 | — |
| 59. | Example 59 | 41.14 | 38.80 | — |
| 60. | Example 60 | 77.02 | 100.00 | D |
| 61. | Example 61 | 100.00 | 100.00 | B |
| 62. | Example 62 | 38.99 | 97.97 | D |
| 63. | Example 63 | 58.61 | 100.00 | D |
| 64. | Example 64 | 68.85 | 100.00 | D |
| 65. | Example 65 | 74.65 | 63.07 | D |
| 66. | Example 66 | 100.00 | 100.00 | D |
| 67. | Example 67 | 87.06 | 85.74 | B |
| 68. | Example 68 | 84.30 | 80.32 | B |
| 69. | Example 69 | 67.15 | — | D |
| 70. | Example 70 | 97.52 | 98.41 | D |
| 71. | Example 71 | 81.53 | 79.33 | A |
| 72. | Example 72 | 82.15 | — | C |
| 73. | Example 73 | 79.85 | 73.09 | B |
| 74. | Example 74 | 91.14 | 90.27 | B |
| 75. | Example 75 | 1.59 | 12.48 | — |
| 76. | Example 76 | 94.95 | 93.46 | A |
| 77. | Example 77 | 88.90 | 72.12 | C |
| 78. | Example 78 | 55.27 | 89.30 | D |
| 79. | Example 79 | 78.42 | 81.57 | C |
| 80. | Example 80 | 93.99 | 97.51 | B |
| 81. | Example 81 | 93.35 | 81.46 | D |
| 82. | Example 82 | 87.45 | — | B |
| 83. | Example 83 | 74.40 | 99.03 | D |
| 84. | Example 84 | 80.19 | 88.60 | D |
| 85. | Example 85 | 72.52 | 88.02 | D |
| 86. | Example 86 | 74.55 | 89.82 | D |
| 87. | Example 87 | 78.22 | 88.54 | C |
| 88. | Example 88 | 100.00 | 100.00 | B |
| 89. | Example 89 | 100.00 | 100.00 | B |
| 90. | Example 90 | 100.0 | 100.0 | B |
| 91. | Example 91 | 98.29 | 87.07 | B |
| 92. | Example 92 | 75.58 | 76.46 | C |
| 93. | Example 93 | 69.07 | 79.56 | D |
| 94. | Example 94 | 72.54 | 80.05 | D |
| 95. | Example 95 | 71.13 | 62.60 | D |
| 96. | Example 96 | 67.26 | 72.34 | D |
| 97. | Example 97 | 76.71 | 78.15 | D |
| 98. | Example 98 | 86.36 | 93.44 | B |
| 99. | Example 99 | 71.29 | 85.08 | D |
| 100. | Example 100 | 66.65 | 84.82 | D |
| 101. | Example 101 | 69.66 | 88.62 | D |
| 102. | Example 102 | 86.22 | 91.52 | B |
| 103. | Example 103 | 99.43 | 100.00 | A |
| 104. | Example 104 | 99.83 | 96.92 | C |
| 105. | Example 105 | 99.14 | 100.00 | D |
| 106. | Example 106 | 100.00 | 100.00 | B |
| 107. | Example 107 | 70.78 | 61.17 | D |
| 108. | Example 108 | 70.67 | 77.93 | B |
| 109. | Example 109 | 60.48 | 82.13 | D |
| 110. | Example 110 | 60.45 | 63.05 | D |
| 111. | Example 111 | 56.38 | 81.71 | D |
| 112. | Example 112 | 44.11 | — | — |
| 113. | Example 113 | 91.32 | 92.32 | B |
| 114. | Example 114 | 61.01 | 69.90 | D |
| 115. | Example 115 | 76.74 | 80.77 | C |
| 116. | Example 116 | 78.64 | 73.05 | D |
| 117. | Example 117 | 6.79 | 15.55 | — |
| 118. | Example 118 | 78.82 | 77.47 | B |
| 119. | Example 119 | 66.90 | — | D |
| 120. | Example 120 | 64.81 | — | D |
| 121. | Example 121 | 70.18 | — | D |
| 122. | Example 122 | 72.74 | 75.40 | D |
| 123. | Example 123 | 75.46 | 76.15 | D |
| 124. | Example 124 | 69.29 | 81.90 | D |
| 125. | Example 125 | 58.39 | 66.75 | — |
| 126. | Example 126 | 63.42 | 76.29 | D |
| 127. | Example 127 | 87.55 | 89.50 | A |
| 128. | Example 128 | 88.06 | 92.13 | B |
| 129. | Example 129 | 85.06 | 74.42 | B |
| 130. | Example 130 | 69.56 | 98.52 | D |
| 131. | Example 131 | 92.42 | 90.23 | A |
| 132. | Example 132 | 74.75 | 88.71 | D |
| 133. | Example 133 | 77.29 | 88.85 | D |
| 134. | Example 134 | 57.08 | 88.21 | D |
| 135. | Example 135 | 71.01 | 90.72 | D |
| 136. | Example 136 | 85.02 | 87.54 | A |
| 137. | Example 137 | 89.51 | 86.30 | B |
| 138. | Example 138 | 92.59 | 85.00 | D |
| 139. | Example 139 | 98.98 | 100.00 | B |
| 140. | Example 140 | 100.00 | 100.00 | B |
| 141. | Example 141 | 77.89 | 95.10 | D |
| 142. | Example 142 | 80.42 | 69.91 | D |
| 143. | Example 143 | 73.11 | — | C |
| 144. | Example 144 | 83.58 | 63.84 | B |
| 145. | Example 145 | 90.05 | 93.20 | D |
| 146. | Example 146 | 94.02 | 81.83 | C |
| 147. | Example 147 | 94.28 | 64.86 | C |
| 148. | Example 148 | 94.34 | 85.26 | D |
| 149. | Example 149 | 79.12 | 74.69 | D |
| 150. | Example 150 | 67.12 | 84.36 | D |
| 151. | Example 151 | 81.09 | 76.20 | D |
| 152. | Example 152 | 93.53 | 89.94 | C |
| 153. | Example 153 | 100.00 | 93.00 | D |
| 154. | Example 154 | 99.58 | 100.00 | D |
| 155. | Example 155 | 100.00 | 93.33 | A |
| 156. | Example 156 | 97.44 | 100.00 | A |
| 157. | Example 157 | 95.18 | 98.57 | B |
| 158. | Example 158 | 100.00 | 100.00 | A |
| 159. | Example 159 | 71.87 | — | D |
| 160. | Example 160 | 80.68 | 68.90 | D |
| 161. | Example 161 | 71.63 | — | D |
| 162. | Example 162 | 74.78 | — | D |
| 163. | Example 163 | 72.82 | — | D |
| 164. | Example 164 | — | — | D |
| 165. | Example 165 | 99.90 | 80.06 | A |
| 166. | Example 166 | 96.36 | 95.51 | A |
| 167. | Example 167 | 85.97 | 71.84 | B |
| 168. | Example 168 | 94.94 | 93.46 | B |
| 169. | Example 169 | 83.70 | — | B |
| 170. | Example 170 | 74.41 | — | D |
| 171. | Example 171 | 84.86 | 79.58 | B |
| 172. | Example 172 | 80.91 | — | D |
| 173. | Example 173 | 76.09 | 69.71 | D |
| 174. | Example 174 | 85.59 | — | C |
| 175. | Example 175 | 84.02 | 81.35 | C |
| 176. | Example 176 | 81.35 | — | D |
| 177. | Example 177 | 68.57 | 50.1 | D |
| 178. | Example 178 | 89.05 | — | D |
| 179. | Example 179 | 92.31 | — | C |
| 180. | Example 180 | 100.00 | 100.00 | A |
| 181. | Example 181 | 100.00 | 100.00 | B |
| 182. | Example 182 | 100.00 | 100.00 | B |
| 183. | Example 183 | 84.15 | 97.11 | C |
| 184. | Example 184 | 78.81 | 81.62 | D |
| 185. | Example 185 | 80.11 | 78.21 | B |
| 186. | Example 186 | 71.87 | 81.24 | D |
| 187. | Example 187 | 76.97 | 77.80 | C |
| 188. | Example 188 | 96.49 | 100.00 | C |
| 189. | Example 189 | 99.22 | 98.58 | A |
| 190. | Example 190 | 93.08 | 100.00 | A |
| 191. | Example 191 | 91.21 | 97.81 | B |
| 192. | Example 192 | 90.61 | 95.35 | B |
| 193. | Example 193 | 84.68 | 75.41 | A |
| 194. | Example 194 | 98.30 | 95.95 | B |
| 195. | Example 195 | 97.71 | 100.00 | C |
| 196. | Example 196 | 100.00 | 100.00 | C |
| 197. | Example 197 | 100.00 | 100.00 | C |
| 198. | Example 198 | 87.71 | 86.69 | B |
| 199. | Example 199 | 86.82 | 93.52 | C |
| 200. | Example 200 | 91.44 | 90.65 | A |
| 201. | Example 201 | 74.01 | 86.97 | D |
| 202. | Example 202 | 82.82 | 77.97 | C |
| 203. | Example 203 | 82.5 | 77.72 | B |
| 204. | Example 204 | 95.18 | 99.73 | C |
| 205. | Example 205 | 95.86 | 94.59 | B |
| 206. | Example 206 | 94.59 | 91.35 | B |
| 207. | Example 207 | 87.82 | 88.20 | B |
| 208. | Example 208 | 82.47 | 71.61 | C |
| 209. | Example 209 | 89.50 | 89.75 | B |
| 210. | Example 210 | 90.97 | 86.35 | D |
| 211. | Example 211 | 92.36 | 93.68 | B |
| 212. | Example 212 | 81.47 | 71.71 | D |
| 213. | Example 213 | 86.92 | 75.56 | D |
| 214. | Example 214 | 81.82 | 85.03 | D |
| 215. | Example 215 | 78.83 | 87.35 | C |
| 216. | Example 216 | 73.36 | 88.34 | D |
| 217. | Example 217 | 83.82 | 89.71 | C |
| 218. | Example 218 | 85.55 | 90.06 | B |
| 219. | Example 219 | 81.73 | 89.20 | D |
| 220. | Example 220 | 87.57 | 100.00 | D |
| 221. | Example 221 | 84.02 | 97.14 | D |
| 222. | Example 222 | 86.16 | 92.72 | D |
| 223. | Example 223 | 89.66 | 96.59 | C |
| 224. | Example 224 | — | — | — |
| 225. | Example 225 | 80.07 | 90.68 | D |
| 226. | Example 226 | 22.93 | 66.93 | — |
| 227. | Example 227 | 6.03 | 16.02 | — |
| 228. | Example 228 | 8.66 | 11.03 | — |
| 229. | Example 229 | 4.45 | 5.81 | — |
| 230. | Example 230 | 3.28 | 5.83 | — |
| 231. | Example 231 | 12.52 | 9.39 | — |
| 232. | Example 232 | 85.06 | 84.85 | B |
| 233. | Example 233 | 32.79 | 69.74 | — |
| 234. | Example 234 | 25.80 | 70.17 | — |
| 235. | Example 235 | 72.86 | — | D |
| 236. | Example 236 | 84.80 | 87.68 | B |
| 237. | Example 237 | 60.88 | 74.30 | D |
| 238. | Example 238 | 80.17 | 71.20 | D |
| 239. | Example 239 | 84.59 | 77.77 | D |
| 240. | Example 240 | — | — | — |
| 241. | Example 241 | — | — | — |
| 242. | Example 242 | — | — | — |
| 243. | Example 243 | — | — | D |
| 244. | Example 244 | — | — | — |
| 245. | Example 245 | — | — | — |
| 246. | Example 246 | — | — | — |
| 247. | Example 247 | — | — | — |
| 248. | Example 248 | — | — | — |
| 249. | Example 249 | — | — | — |
| 250. | Example 250 | — | — | — |
| 251. | Example 251 | — | — | — |
| 252. | Example 252 | — | — | — |
| 253. | Example 253 | — | — | — |
| 254. | Example 254 | — | — | — |
| 255. | Example 255 | — | — | — |
| 256. | Example 256 | — | — | — |
| 257. | Example 257 | — | — | — |

(—): Not determined

1-37. (canceled)

38. A compound selected from:
6-(2-Chlorophenyl)-5-hydroxy-1,3-dimethyl-1,7-dihydro-4H-pyrazolo[3,4-b]pyridin-4-one;
6-(2-Chlorophenyl)-5-hydroxy-3-methyl-1-(2-morpholinoethyl)-1H-pyrazolo[3,4-b]pyridin-4(7H)-one;
6-(2-Chlorophenyl)-5-hydroxy-1-methyl-1,7-dihydro-4H-pyrazolo[3,4-b]pyridin-4-one;
6-(3-Chlorophenyl)-5-hydroxy-1-methyl-1,7-dihydro-4H-pyrazolo[3,4-b]pyridin-4-one;
6-(2,4-Dichlorophenyl)-5-hydroxy-1-methyl-1,7-dihydro-4H-pyrazolo[3,4-b]pyridin-4-one;
6-[4-Fluoro-3-(trifluoromethyl)phenyl]-5-hydroxy-1-methyl-1,7-dihydro-4H-pyrazolo[3,4-b]pyridin-4-one;
6-(2-Chloro-6-fluorophenyl)-5-hydroxy-1-methyl-1,7-dihydro-4H-pyrazolo[3,4-b]pyridin-4-one;
6-(2-Chlorophenyl)-5-hydroxy-1-(2,2,2-trifluoroethyl)-1,7-dihydro-4H-pyrazolo[3,4-b]pyridin-4-one;
6-(2,6-Difluorophenyl)-1-(4-fluorophenyl)-5-hydroxy-1,7-dihydro-4H-pyrazolo[3,4-b]pyridin-4-one;
6-(2-Chlorophenyl)-1-(4-fluorophenyl)-5-hydroxy-1,7-dihydro-4H-pyrazolo[3,4-b]pyridin-4-one;
6-(2,4-Dichlorophenyl)-5-hydroxy-1,3-dimethyl-1,7-dihydro-4H-pyrazolo[3,4-b]pyridin-4-one;
6-(2-Chloro-4-fluorophenyl)-5-hydroxy-1,3-dimethyl-1,7-dihydro-4H-pyrazolo[3,4-b]pyridin-4-one;
6-(2,6-Difluorophenyl)-5-hydroxy-1,3-dimethyl-1,7-dihydro-4H-pyrazolo[3,4-b] pyridin-4-one;
6-(2,4-Difluorophenyl)-5-hydroxy-1,3-dimethyl-1,7-dihydro-4H-pyrazolo[3,4-b] pyridin-4-one;
6-(3,4-Dimethylphenyl)-5-hydroxy-1,3-dimethyl-1,7-dihydro-4H-pyrazolo[3,4-b] pyridin-4-one;
6-[3-Fluoro-4-(trifluoromethoxy)phenyl]-5-hydroxy-1,3-dimethyl-1,7-dihydro-4H-pyrazolo[3,4-b]pyridin-4-one;
6-(3,4-Difluorophenyl)-5-hydroxy-1,3-dimethyl-1,7-dihydro-4H-pyrazolo[3,4-b] pyridin-4-one;
6-(2-Chloro-4-methoxyphenyl)-5-hydroxy-1,3-dimethyl-1,7-dihydro-4H-pyrazolo[3,4-b]pyridin-4-one;
6-(2-Fluoro-4-methoxyphenyl)-5-hydroxy-1,3-dimethyl-1,7-dihydro-4H-pyrazolo[3,4-b]pyridin-4-one;
6-(2,5-Dichlorophenyl)-5-hydroxy-1,3-dimethyl-1,7-dihydro-4H-pyrazolo[3,4-b]pyridin-4-one;
6-[2-Fluoro-4-(trifluoromethyl)phenyl]-5-hydroxy-1,3-dimethyl-1,7-dihydro-4H-pyrazolo[3,4-b]pyridin-4-one;
6-[3-Fluoro-4-(trifluoromethyl)phenyl]-5-hydroxy-1,3-dimethyl-1,7-dihydro-4H-pyrazolo[3,4-b]pyridin-4-one;
6-(2-Chloro-5-methoxyphenyl)-5-hydroxy-1,3-dimethyl-1,7-dihydro-4H-pyrazolo[3,4-b]pyridin-4-one;
6-[4-Chloro-3-(trifluoromethyl)phenyl]-5-hydroxy-1,3-dimethyl-1,7-dihydro-4H-pyrazolo[3,4-b] pyridin-4-one;
6-(4-Chloro-2-fluorophenyl)-5-hydroxy-1,3-dimethyl-1,7-dihydro-4H-pyrazolo[3,4-b]pyridin-4-one;
6-(2-Chlorophenyl)-1-ethyl-5-hydroxy-3-methyl-1,7-dihydro-4H-pyrazolo[3,4-b]pyridin-4-one;
6-(2-Chlorophenyl)-5-hydroxy-3-methyl-1-(2,2,2-trifluoroethyl)-1,7-dihydro-4H-pyrazolo[3,4-b] pyridin-4-one;
6-(2-Chlorophenyl)-5-hydroxy-3-methyl-1-(propan-2-yl)-1,7-dihydro-4H-pyrazolo[3,4-b]pyridin-4-one;
6-(2,6-Difluorophenyl)-5-hydroxy-1-(4-methoxyphenyl)-3-methyl-1,7-dihydro-4H-pyrazolo[3,4-b]pyridin-4-one;
6-(2-Chlorophenyl)-5-hydroxy-3-methyl-1-(pyridin-2-yl)-1,7-dihydro-4H-pyrazolo[3,4-b]pyridin-4-one;
6-(2-Chlorophenyl)-1-(3,4-difluorophenyl)-5-hydroxy-3-methyl-1,7-dihydro-4H-pyrazolo[3,4-b]pyridin-4-one;
6-(2,6-Difluorophenyl)-1-(4-fluorophenyl)-5-hydroxy-3-methyl-1,7-dihydro-4H-pyrazolo[3,4-b]pyridin-4-one;
6-(2-Chlorophenyl)-1-(4-fluorophenyl)-5-hydroxy-3-methyl-1,7-dihydro-4H-pyrazolo[3,4-b]pyridin-4-one;
6-(2-Chlorophenyl)-1-(3-fluorophenyl)-5-hydroxy-3-methyl-1,7-dihydro-4H-pyrazolo[3,4-b]pyridin-4-one;
6-(2,6-Difluorophenyl)-1-(3-fluorophenyl)-5-hydroxy-3-methyl-1,7-dihydro-4H-pyrazolo[3,4-b]pyridin-4-one;
6-(2,6-Difluorophenyl)-5-hydroxy-3-methyl-1-(pyridin-2-yl)-1,7-dihydro-4H-pyrazolo[3,4-b]pyridin-4-one;
6-(2,6-Difluorophenyl)-1-(3,4-difluorophenyl)-5-hydroxy-3-methyl-1,7-dihydro-4H-pyrazolo[3,4-b]pyridin-4-one;
6-(2-Fluoro-4-methoxyphenyl)-1-(4-fluorophenyl)-5-hydroxy-3-methyl-1,7-dihydro-4H-pyrazolo[3,4-b]pyridin-4-one;
1-(3,4-Difluorophenyl)-6-(2-fluoro-4-methoxyphenyl)-5-hydroxy-3-methyl-1,7-dihydro-4H-pyrazolo[3,4-b]pyridin-4-one;
6-(2-Chlorophenyl)-5-hydroxy-1-methyl-3-(trifluoromethyl)-1,7-dihydro-4H-pyrazolo[3,4-b]pyridin-4-one;
6-(2-Fluorophenyl)-5-hydroxy-1-methyl-3-(trifluoromethyl)-1,7-dihydro-4H-pyrazolo[3,4-b]pyridin-4-one;
6-(4-Fluorophenyl)-5-hydroxy-1-methyl-3-(trifluoromethyl)-1,7-dihydro-4H-pyrazolo[3,4-b]pyridin-4-one;
6-(4-Chlorophenyl)-5-hydroxy-1-methyl-3-(trifluoromethyl)-1,7-dihydro-4H-pyrazolo[3,4-b]pyridin-4-one;
6-(2-Chloro-4-fluorophenyl)-5-hydroxy-1-methyl-3-(trifluoromethyl)-1,7-dihydro-4 H pyrazolo[3,4-b]pyridin-4-one;
6-(2-Chloro-6-fluorophenyl)-5-hydroxy-1-methyl-3-(trifluoromethyl)-1,7-dihydro-4H-pyrazolo[3,4-b]pyridin-4-one;
6-(3-Chloropyridin-4-yl)-5-hydroxy-1-methyl-3-(trifluoromethyl)-1,7-dihydro-4H-pyrazolo[3,4-b]pyridin-4-one hydrochloride;
6-(2-Fluoro-4-methoxyphenyl)-5-hydroxy-1-methyl-3-(trifluoromethyl)-1,7-dihydro-4H-pyrazolo[3,4-b]pyridin-4-one;
6-(2-Chloro-4-methoxyphenyl)-5-hydroxy-1-methyl-3-(trifluoromethyl)-1,7-dihydro-4H-pyrazolo[3,4-b]pyridin-4-one;
6-(2-Chloro-5-methoxyphenyl)-5-hydroxy-1-methyl-3-(trifluoromethyl)-1,7-dihydro-4H-pyrazolo[3,4-b]pyridin-4-one;
6-(2,5-Dichlorophenyl)-5-hydroxy-1-methyl-3-(trifluoromethyl)-1,7-dihydro-4H-pyrazolo[3,4-b]pyridin-4-one;
6-(2,4-Dimethoxyphenyl)-5-hydroxy-1-methyl-3-(trifluoromethyl)-1,7-dihydro-4H-pyrazolo[3,4-b]pyridin-4-one;
6-(4-Chloro-2-fluorophenyl)-5-hydroxy-1-methyl-3-(trifluoromethyl)-1,7-dihydro-4H-pyrazolo[3,4-b]pyridin-4-one;
5-Hydroxy-6-(4-methoxyphenyl)-1-methyl-3-(trifluoromethyl)-1,7-dihydro-4H-pyrazolo[3,4-b]pyridin-4-one;
5-Hydroxy-6-[4-(1H-imidazol-1-yl)phenyl]-1-methyl-3-(trifluoromethyl)-1,7-dihydro-4H-pyrazolo[3,4-b]pyridin-4-one;
5-Hydroxy-1-methyl-6-(pyridin-4-yl)-3-(trifluoromethyl)-1,7-dihydro-4H-pyrazolo[3,4-b]pyridin-4-one;
6-(2-Chlorophenyl)-3-ethyl-5-hydroxy-1-methyl-1,7-dihydro-4H-pyrazolo[3,4-b]pyridin-4-one;
6-(2-Chlorophenyl)-3-(2-fluorobenzyl)-5-hydroxy-2-methyl-2,7-dihydro-4H-pyrazolo[3,4-b]pyridin-4-one;
6-(2-Chlorophenyl)-3-(4-fluorophenyl)-5-hydroxy-2-methyl-2,7-dihydro-4H-pyrazolo[3,4-b]pyridin-4-one;
6-(2-Chlorophenyl)-5-hydroxy-2,3-dimethyl-2,7-dihydro-4H-pyrazolo[3,4-b]pyridin-4-one;
6-(2-Chlorophenyl)-5-hydroxy-2-methylthieno[2,3-b]pyridin-4(7H)-one;
6-[4-Fluoro-3-(trifluoromethyl)phenyl]-5-hydroxy-2-methylthieno[2,3-b]pyridin-4(7H)-one;
5-(2-Chlorophenyl)-6-hydroxy-2-methyl[1,3]thiazolo[5,4-b]pyridin-7(4H)-one;
5-(2-Chlorophenyl)-6-hydroxy-2-trifluoromethyl[1,3]thiazolo[5,4-b]pyridin-7(4H)-one;
6-(2-Chlorophenyl)-5-hydroxy-3-methyl[1,2] oxazolo[5,4-b]pyridin-4(7H)-one;
6-(2-Chloro-4-(2-methoxyethoxy)phenyl)-5-hydroxy-1,3-dimethyl-1H-pyrazolo[3,4-b]pyridin-4(7H)-one;
6-(2-Fluoro-4-(2-methoxyethoxy)phenyl)-5-hydroxy-1-methyl-3-(trifluoromethyl)-1H-pyrazolo[3,4-b]pyridin-4(7H)-one;
6-(2,6-Difluoro-4-methoxyphenyl)-5-hydroxy-1,3-dimethyl-1H-pyrazolo[3,4-b]pyridin-4(7H)-one;
6-(2,6-Difluorophenyl)-1-ethyl-5-hydroxy-3-methyl-1H-pyrazolo[3,4-b]pyridin-4(7H)-one;
1-Ethyl-6-(2-fluoro-4-methoxyphenyl)-5-hydroxy-3-methyl-1H-pyrazolo[3,4-b]pyridin-4(7H)-one;
6-(2-Fluoro-4-methoxyphenyl)-5-hydroxy-3-methyl-1-(2,2,2-trifluoroethyl)-1H-pyrazolo[3,4-b]pyridin-4(7H)-one;
6-(2,6-Difluorophenyl)-3-ethyl-5-hydroxy-1-methyl-1H-pyrazolo[3,4-b]pyridin-4(7H)-one;
3-Ethyl-6-(2-fluoro-4-methoxyphenyl)-5-hydroxy-1-methyl-1H-pyrazolo[3,4-b]pyridin-4(7H)-one;
6-(2-Chloro-4-methoxyphenyl)-3-ethyl-5-hydroxy-1-methyl-1H-pyrazolo[3,4-b]pyridin-4(7H)-one;
6-(2,6-Difluorophenyl)-5-hydroxy-1-isopropyl-3-methyl-1H-pyrazolo[3,4-b]pyridin-4(7H)-one;
6-(2-Chlorophenyl)-5-hydroxy-3-isopropyl-2-methyl-2H-pyrazolo[3,4-b]pyridin-4(7H)-one;
1-Benzyl-6-(2,6-difluorophenyl)-5-hydroxy-3-methyl-1H-pyrazolo[3,4-b]pyridin-4(7H)-one;
1-Benzyl-6-(2-fluoro-4-methoxyphenyl)-5-hydroxy-3-methyl-1H-pyrazolo[3,4-b]pyridin-4(7H)-one;
5-(2-Chlorophenyl)-6-hydroxy-7-oxo-4,7-dihydroisothiazolo[4,5-b]pyridine-3-carboxamide;
6-(2,6-Difluorophenyl)-5-hydroxy-3-methyl-1-propyl-1H-pyrazolo[3,4-b]pyridin-4(7H)-one;
6-(2-Chlorophenyl)-1-(2-(dimethylamino)ethyl)-5-hydroxy-3-methyl-1H-pyrazolo[3,4-b]pyridin-4(7H)-one;
6-(2,5-Difluorophenyl)-1-ethyl-5-hydroxy-3-methyl-1H-pyrazolo[3,4-b]pyridin-4(7H)-one;
6-(2-Fluoro-4-methoxyphenyl)-5-hydroxy-3-methyl-1-propyl-1H-pyrazolo[3,4-b]pyridin-4(7H)-one;
5-(2-Chlorophenyl)-6-hydroxy-1,3-dimethyl-1H-pyrazolo[4,3-b]pyridin-7(4H)-one;
6-(2,6-Difluorophenyl)-1-(4-fluorobenzyl)-5-hydroxy-3-methyl-1H-pyrazolo[3,4-b]pyridin-4 (7H)-one;
6-(2-Chlorophenyl)-3-(difluoromethyl)-5-hydroxy-1-methyl-1H-pyrazolo[3,4-b]pyridin-4(7H)-one;
3-(Difluoromethyl)-6-(2,6-difluorophenyl)-5-hydroxy-1-methyl-1H-pyrazolo[3,4-b]pyridin-4 (7H)-one;
1-Cyclopropyl-6-(2,6-difluorophenyl)-5-hydroxy-3-methyl-1H-pyrazolo[3,4-b]pyridin-4(7H)-one;
6-(2,6-Difluorophenyl)-5-hydroxy-3-methyl-1-(2-morpholinoethyl)-1H-pyrazolo[3,4-b]pyridin-4(7H)-one;
6-(2,6-Difluorophenyl)-1-(2-(dimethylamino)ethyl)-5-hydroxy-3-methyl-1H-pyrazolo[3,4-b]pyridin-4(7H)-one;
6-(2,6-Difluorophenyl)-5-hydroxy-1-(2-methoxyethyl)-3-methyl-1H-pyrazolo[3,4-b]pyridin-4(7H)-one;
6-(2,6-Difluorophenyl)-5-hydroxy-3-isopropyl-1-methyl-1H-pyrazolo[3,4-b]pyridin-4(7H)one;
6-(2,6-Difluorophenyl)-5-hydroxy-1-(3-methoxypropyl)-3-methyl-1H-pyrazolo[3,4-b]pyridin-4(7H)-one;
1-Cyclopropyl-6-(2,6-difluoro-3-methylphenyl)-5-hydroxy-3-methyl-1H-pyrazolo[3,4-b]pyridin-4(7H)-one;
1-Cyclopropyl-5-hydroxy-3-methyl-6-(2,4,6-trifluorophenyl)-1H-pyrazolo[3,4-b]pyridin-4(7H)-one;
6-(2,3-Difluorophenyl)-1-ethyl-5-hydroxy-3-methyl-1H-pyrazolo[3,4-b]pyridin-4(7H)-one;
1-Cyclopropyl-6-(2,3-difluorophenyl)-5-hydroxy-3-methyl-1H-pyrazolo[3,4-b]pyridin-4(7H)-one;
1-Ethyl-5-hydroxy-3-methyl-6-(2,4,6-trifluorophenyl)-1H-pyrazolo[3,4-b]pyridin-4(7H)-one;
6-(2,6-Difluorophenyl)-5-hydroxy-3-(trifluoromethyl)-1H-pyrazolo[3,4-b]pyridin-4(7H)-one;
3-(Difluoromethyl)-6-(2,6-difluorophenyl)-1-ethyl-5-hydroxy-1H-pyrazolo[3,4-b]pyridin-4(7H)-one;
6-(2,6-Difluorophenyl)-1-ethyl-5-hydroxy-3-(trifluoromethyl)-1H-pyrazolo[3,4-b]pyridin-4(7H)-one;
5-(2,6-Difluorophenyl)-3-ethyl-6-hydroxy-3H-imidazo[4,5-b]pyridin-7(4H)-one;
6-(2,6-Difluorophenyl)-5-hydroxy-3-(methoxymethyl)-1-methyl-1H-pyrazolo[3,4-b]pyridin-4(7H)-one;
6-(2,6-Difluorophenyl)-1,3-diethyl-5-hydroxy-1,7-dihydro-4H-pyrazolo[3,4-b]pyridin-4-one;
1-Ethyl-6-[2-fluoro-3-(trifluoromethyl)phenyl]-5-hydroxy-3-methyl-1,7-dihydro-4H-pyrazolo[3,4-b]pyridin-4-one;
6-(2,6-Difluorophenyl)-1-ethyl-5-hydroxy-1,7-dihydro-4H-pyrazolo[3,4-b]pyridin-4-one;
6-(2,6-Difluorophenyl)-5-hydroxy-3-methyl-1,7-dihydro-4H-pyrazolo[3,4-b]pyridin-4-one;
1-Ethyl-6-(2-fluorophenyl)-5-hydroxy-3-methyl-1,7-dihydro-4H-pyrazolo[3,4-b]pyridin-4-one;
6-(2,6-Difluorophenyl)-5-hydroxy-1-isobutyl-3-methyl-1H-pyrazolo[3,4-b]pyridin-4(7H)-one;
6-(2-Chlorophenyl)-1-ethyl-5-hydroxy-3-(trifluoromethyl)-1H-pyrazolo[3,4-b]pyridin-4(7H)-one;
1-Ethyl-5-hydroxy-6-(2-methoxyphenyl)-3-methyl-1H-pyrazolo[3,4-b]pyridin-4(7H)-one;
6-(2,6-Difluorophenyl)-5-hydroxy-1-isobutyl-3-(trifluoromethyl)-1H-pyrazolo[3,4-b]pyridin-4 (7H)-one;
1-Ethyl-6-(4-fluorophenyl)-5-hydroxy-3-methyl-1,7-dihydro-4H-pyrazolo[3,4-b]pyridin-4-one;
6-(2,6-Difluorophenyl)-3-(4-fluorophenyl)-5-hydroxy-1-methyl-1H-pyrazolo[3,4-b]pyridin-4 (7H)-one;
1-Cyclopropyl-6-(2-fluoro-4-methoxyphenyl)-5-hydroxy-3-methyl-1H-pyrazolo[3,4-b]pyridin-4(7H)-one;
3-Benzyl-6-(2,6-difluorophenyl)-5-hydroxy-1-methyl-1H-pyrazolo[3,4-b]pyridin-4(7H)-one;
6-(2,4-Difluoro-3-(trifluoromethyl)phenyl)-1-ethyl-5-hydroxy-3-methyl-1H-pyrazolo[3,4-b]pyridin-4(7H)-one;
6-(2,6-Difluorophenyl)-5-hydroxy-1-methyl-3-morpholino-1H-pyrazolo[3,4-b]pyridin-4(7H)-one;
6-(2,6-Difluoro-4-methoxyphenyl)-1-ethyl-5-hydroxy-3-methyl-1H-pyrazolo[3,4-b]pyridin-4 (7H)-one;
6-(4-Amino-2-fluorophenyl)-1-ethyl-5-hydroxy-3-methyl-1,7-dihydro-4H-pyrazolo[3,4-b]pyridin-4-one;
1-Ethyl-6-[2-fluoro-4-(2-methoxyethoxy)phenyl]-5-hydroxy-3-methyl-1,7-dihydro-4H-pyrazolo[3,4-b]pyridin-4-one;
6-[4-(Cyclopropylmethoxy)-2-fluorophenyl] -1-ethyl-5-hydroxy-3-methyl-1,7-dihydro-4H-pyrazolo[3,4-b]pyridin-4-one;
6-(2,6-Difluorophenyl)-1-ethyl-5-hydroxy-3-(2-methylpropyl)-1,7-dihydro-4H-pyrazolo[3,4-b]pyridin-4-one;
6-(2-Chloro-6-fluorophenyl)-1-ethyl-5-hydroxy-3-methyl-1,7-dihydro-4H-pyrazolo[3,4-b]pyridin-4-one;
6-(2-Chloro-6-fluorophenyl)-1-cyclopropyl-5-hydroxy-3-methyl-1,7-dihydro-4H-pyrazolo[3,4-b]pyridin-4-one;
6-(2-Chlorophenyl)-1-cyclopropyl-5-hydroxy-3-methyl-1,7-dihydro-4H-pyrazolo[3,4-b]pyridin-4-one;
6-(2-Chlorophenyl)-1-(4-fluoro-2-methylphenyl)-5-hydroxy-3-methyl-1,7-dihydro-4H-pyrazolo[3,4-b]pyridin-4-one;
1-Cyclopentyl-6-(2,6-difluorophenyl)-5-hydroxy-3-methyl-1,7-dihydro-4H-pyrazolo[3,4-b]pyridin-4-one;
6-(2,6-Difluorophenyl)-5-hydroxy-3-methyl-1-(tetrahydro-2H-pyran-4-yl)-1,7-dihydro-4H-pyrazolo[3,4-b]pyridin-4-one;
N-(4-Chloro-3-(1-ethyl-5-hydroxy-3-methyl-4-oxo-4,7-dihydro-1H-pyrazolo[3,4-b]pyridin-6-yl)benzyl)pivalamide;
6-(2-Chlorophenyl)-5-hydroxy-1-(tetrahydro-2H-pyran-4-yl)-3-(trifluoromethyl)-1H-pyrazolo[3,4-b]pyridin-4(7H)-one;
1-Cyclobutyl-6-(2,6-difluorophenyl)-5-hydroxy-3-methyl-1H-pyrazolo[3,4-b]pyridin-4(7H)-one;
N-(4-Chloro-3-(5-hydroxy-1-methyl-4-oxo-3-(trifluoromethyl)-4,7-dihydro-1H-pyrazolo[3,4-b]pyridin-6-yl)benzyl)pivalamide;
N-(4-Chloro-3-(1-cyclopropyl-5-hydroxy-3-methyl-4-oxo-4,7-dihydro-1H-pyrazolo[3,4-b]pyridin-6-yl)benzyl)pivalamide;
6-(2,6-Difluorophenyl)-5-hydroxy-1-(tetrahydro-2H-pyran-4-yl)-3-(trifluoromethyl)-1,7-dihydro-4H-pyrazolo[3,4-b]pyridin-4-one;
N-(4-Fluoro-3-(5-hydroxy-1-methyl-4-oxo-3-(trifluoromethyl)-4,7-dihydro-1H-pyrazolo[3,4-b]pyridin-6-yl)benzyl)pivalamide;
1-Cyclohexyl-6-(2,6-difluorophenyl)-5-hydroxy-3-methyl-1,7-dihydro-4H-pyrazolo[3,4-b]pyridin-4-one;
1-(4,4-Difluorocyclohexyl)-6-(2,6-difluorophenyl)-5-hydroxy-3-methyl-1H-pyrazolo[3,4-b]pyridin-4(7H)-one;
N-(3-(1-Ethyl-5-hydroxy-3-methyl-4-oxo-4,7-dihydro-1H-pyrazolo[3,4-b]pyridin-6-yl)-4-fluorobenzyl)pivalamide;
1-(2-Chloro-4-fluorophenyl)-6-(2,6-difluorophenyl)-5-hydroxy-3-methyl-1H-pyrazolo[3,4-b]pyridin-4(7H)-one;
1-Cyclopropyl-6-(2,6-difluorophenyl)-3-ethyl-5-hydroxy-1H-pyrazolo[3,4-b]pyridin-4(7H)-one;
1-(2-Chloro-4-fluorophenyl)-6-(2-chlorophenyl)-5-hydroxy-3-methyl-1H-pyrazolo[3,4-b]pyridin-4(7H)-one;
N-(4-Chloro-3-(5-hydroxy-3-methyl-4-oxo-1-(tetrahydro-2H-pyran-4-yl)-4 ,7-dihydro-1H-pyrazolo[3,4-b]pyridin-6-yl)benzyl)pivalamide;
1-Ethyl-5-hydroxy-3-methyl-6-(2-(trifluoromethyl)phenyl)-1H-pyrazolo[3,4-b]pyridin-4(7H)-one;
6-(2-Chlorophenyl)-1-cyclobutyl-5-hydroxy-3-methyl-1H-pyrazolo[3,4-b]pyridin-4(7H)-one;
3-(2-Chlorobenzyl)-6-(2-chlorophenyl)-5-hydroxy-1-methyl-1H-pyrazolo[3,4-b]pyridin-4(7H)-one;
6-(2-Chlorophenyl)-3-ethyl-5-hydroxy-1-(2-morpholinoethyl)-1H-pyrazolo[3,4-b]pyridin-4(7H)-one;
6-(2-Chlorophenyl)-5-hydroxy-3-methyl-1-(3-morpholinopropyl)-1H-pyrazolo[3,4-b]pyridin-4(7H)-one;
6-(2-Chlorophenyl)-1-(2-((2S ,6R)-2,6-dimethylmorpholino)ethyl)-5-hydroxy-3-methyl-1H-pyrazolo[3,4-b]pyridin-4(7H)-one;
6-(2-Chlorophenyl)-5-hydroxy-3-methyl-1-(2-(piperidin-1-yl)ethyl)-1H-pyrazolo[3,4-b]pyridin-4(7H)-one;
6-(2-Chlorophenyl)-5-hydroxy-1-(2-morpholinoethyl)-3-(trifluoromethyl)-1H-pyrazolo[3,4-b]pyridin-4(7H)-one;
6-(2-Chlorophenyl)-5-hydroxy-3-methyl-1-(2-(pyrrolidin-1-yl)ethyl)-1H-pyrazolo[3,4-b]pyridin-4(7H)-one;
5-Hydroxy-3-methyl-1-(2-morpholinoethyl)-6-(2-(trifluoromethyl)phenyl)-1H-pyrazolo[3,4-b]pyridin-4(7H)-one;
6-(2-fluoro-3-(trifluoromethyl)phenyl)-5-hydroxy-3-methyl-1-(2-morpholinoethyl)-1H-pyrazolo[3,4-b]pyridin-4(7H)-one;
6-(2-chlorophenyl)-1-(2-(dimethylamino)ethyl)-5-hydroxy-3-(trifluoromethyl)-1H-pyrazolo[3,4-b]pyridin-4(7H)-one;
6-(2-chlorophenyl)-5-hydroxy-3-methyl-1-(2-(4-methylpiperazin-1-yl)ethyl)-1H-pyrazolo[3,4-b]pyridin-4(7H)-one;
6-(2,6-difluorophenyl)-3-ethyl-5-hydroxy-1-(2-morpholinoethyl)-1H-pyrazolo[3,4-b]pyridin-4(7H)-one;
1-(2-(1H-pyrazol-1-yl)ethyl)-6-(2-chlorophenyl)-3-ethyl-5-hydroxy-1H-pyrazolo[3,4-b]pyridin-4(7H)-one;
1-(2-(1H-pyrazol-1-yl)ethyl)-6-(2,6-difluorophenyl)-3-ethyl-5-hydroxy-1H-pyrazolo[3,4-b]pyridin-4(7H)-one;
3-Ethyl-6-(4-fluoro-3-(trifluoromethyl)phenyl)-5-hydroxy-1-methyl-1H-pyrazolo[3,4-b]pyridine-4(7H)-one;
3-Ethyl-6-(2-fluoro-3-(trifluoromethyl)phenyl)-5-hydroxy-1-methyl-1H-pyrazolo[3,4-b]pyridine-4(7H)-one;
6-(2,4-Difluorophenyl)-3-ethyl-5-hydroxy-1-methyl-1H-pyrazolo[3,4-b]pyridin-4(7H)-one;
6-(3,5-Difluorophenyl)-3-ethyl-5-hydroxy-1-methyl-1H-pyrazolo[3,4-b]pyridine-4(7H)-one;
6-(2,5-Difluorophenyl)-3-ethyl-5-hydroxy-1-methyl-1H-pyrazolo[3,4-b]pyridine-4(7H)-one;
3-Ethyl-6-(2-fluoro-3-(trifluoromethyl)phenyl)-5-hydroxy-1-(2-morpholinoethyl)-1H-pyrazolo[3,4-b]pyridin-4(7H)-one;
6-(2-Chlorophenyl)-3-ethyl-5-hydroxy-1-(2-(4-methylpiperazin-1-yl)ethyl)-1H-pyrazolo[3,4-b]pyridine-4(7H)-one;
6-(2,6-Difluorophenyl)-3-ethyl-5-hydroxy-1-(2-(4-methylpiperazin-1-yl)ethyl)-1H-pyrazolo[3,4-b]pyridine-4(7H)-one;
4-(2-(6-(2-Chlorophenyl)-3-ethyl-5-hydroxy-4-oxo-4,7-dihydro-1H-pyrazolo[3,4-b]pyridin-1-yl)ethyl)morpholin-3-one;
4-(2-(6-(2,6-Difluorophenyl)-3-ethyl-5-hydroxy-4-oxo-4,7-dihydro-1H-pyrazolo[3,4-b]pyridin-1-yl)ethyl)morpholin-3-one;
6-(2,6-Difluorophenyl)-5-hydroxy-3-methyl-1-(2-(4-methylpiperazin-1-yl)ethyl)-1H-pyrazolo[3,4-b]pyridin-4(7H)-one;
6-(2,4-Difluorophenyl)-3-ethyl-5-hydroxy-1-propyl-1H-pyrazolo[3,4-b]pyridin-4(7H)-one;
6-(2,4-Difluorophenyl)-3-ethyl-5-hydroxy-1-propyl-1H-pyrazolo[3,4-b]pyridin-4(7H)-one;
6-(2,6-Difluorophenyl)-3-ethyl-1-(2-(4-ethylpiperazin-1-yl)ethyl)-5-hydroxy-1H-pyrazolo[3,4-b]pyridin-4(7H)-one;
6-(2-Chlorophenyl)-3-ethyl-1-(2-(4-ethylpiperazin-1-yl)ethyl)-5-hydroxy-1H-pyrazolo[3,4-b]pyridin-4(7H)-one;
3-Ethyl-6-(2-fluoro-3-(trifluoromethyl)phenyl)-5-hydroxy-1-(3-morpholinopropyl)-1H-pyrazolo[3,4-b]pyridin-4(7H)-one;
6-(2,6-Difluorophenyl)-3-ethyl-5-hydroxy-1-(3-morpholinopropyl)-1H-pyrazolo[3,4-b]pyridin-4(7H)-one;
6-(2,4-Difluorophenyl)-3-ethyl-5-hydroxy-1-(3-morpholinopropyl)-1H-pyrazolo[3,4-b]pyridin-4(7H)-one;
6-(2-Chlorophenyl)-5-hydroxy-3-methyl-1-(2-(piperazin-1-yl)ethyl)-1H-pyrazolo[3,4-b]pyridin-4(7H)-one;
6-(2-fluoro-3-(trifluoromethyl)phenyl)-5-hydroxy-3-methyl-1-(2-(4-methylpiperazin-1-yl)ethyl)-1H-pyrazolo[3,4-b]pyridin-4(7H)-one;
3-Ethyl-6-(2-fluoro-3-(trifluoromethyl)phenyl)-5-hydroxy-1-(2-(4-methylpiperazin-1-yl)ethyl)-1H-pyrazolo[3,4-b]pyridin-4(7H)-one;
6-(2,6-Difluorophenyl)-3-ethyl-5-hydroxy-1-isopentyl-1H-pyrazolo[3,4-b]pyridin-4(7H)-one;
6-(2-Chlorophenyl)-3-ethyl-5-hydroxy-1-isopentyl-1H-pyrazolo[3,4-b]pyridin-4(7H)-one;
6-(2,6-Difluorophenyl)-5-hydroxy-3-(hydroxymethyl)-1-propyl-1H-pyrazolo[3,4-b]pyridin-4(7H)-one;
1-Cyclopropyl-6-(2,6-difluorophenyl)-5-hydroxy-3-(hydroxymethyl)-1H-pyrazolo[3,4-b]pyridin-4(7H)-one;
4-(2-(6-(2,6-Difluorophenyl)-3-ethyl-5-hydroxy-4-oxo-4,7-dihydro-1H-pyrazolo[3,4-b]pyridin-1-yl)ethyl)-2,2-dimethylmorpholin-3-one;
6-(2,6-Difluorophenyl)-5-hydroxy-1-methyl-3-(1-(methylsulfonyl)piperidin-4-yl)-1H-pyrazolo[3,4-b]pyridin-4(7H)-one;
1-Cyclopropyl-6-(2,6-difluorophenyl)-5-hydroxy-3-(1-(methylsulfonyl)piperidin-4-yl)-1H-pyrazolo[3,4-b]pyridin-4(7H)-one;
6-(2-(2,4-Difluorophenyl)thiazol-5-yl)-3-ethyl-5-hydroxy-1-methyl-1H-pyrazolo[3,4-b]pyridin-4(7H)-one;
6-(2,6-Difluorophenyl)-5-hydroxy-3-(hydroxymethyl)-1-methyl-1H-pyrazolo[3,4-b]pyridin-4(7H)-one;
6-(2,6-Difluorophenyl)-5-hydroxy-1-methyl-3-(tetrahydro-2H-pyran-4-yl)-1H-pyrazolo[3,4-b]pyridin-4(7H)-one;
1-Cyclopropyl-6-(2,6-difluorophenyl)-5-hydroxy-3-(tetrahydro-2H-pyran-4-yl)-1H-pyrazolo[3,4-b]pyridin-4(7H)-one;
6-(2,6-difluorophenyl)-5-hydroxy-1-methyl-3-(morpholinomethyl)-1H-pyrazolo[3,4-b]pyridin-4(7H)-one;
6-(2,6-Difluorophenyl)-5-hydroxy-3-((4-isopropylpiperazin-1-yl)methyl)-1-methyl-1H-pyrazolo[3,4-b]pyridin-4(7H)-one;
6-(2-Chlorophenyl)-5-hydroxy-1-methyl-3-(tetrahydro-2H-pyran-4-yl)-1H-pyrazolo[3,4-b]pyridin-4(7H)-one;
6-(2-chlorophenyl)-5-hydroxy-1-methyl-3-(1-(methylsulfonyl)piperidin-4-yl)-1H-pyrazolo[3,4-b]pyridin-4(7H)-one;
(S)-6-(2,6-Difluorophenyl)-3-((3-fluoropyrrolidin--yl)methyl)-5-hydroxy-1-methyl-1H-pyrazolo[3,4-b]pyridin-4(7H)-one;
6-(2,6-Difluorophenyl)-3-((4,4-difluoropiperidin-1-yl)methyl)-5-hydroxy-1-methyl-1H-pyrazolo[3,4-b]pyridin-4(7H)-one;
6-(2,6-Difluorophenyl)-3-((2,6-dimethylmorpholino)methyl)-5-hydroxy-1-methyl-1H-pyrazolo[3,4-b]pyridin-4(7H)-one;
6-(2-Chlorophenyl)-5-hydroxy-3-(hydroxymethyl)-1-methyl-1H-pyrazolo[3,4-b]pyridin-4(7H)-one;
6-(2-Chlorophenyl)-5-hydroxy-1-methyl-3-(morpholinomethyl)-1H-pyrazolo[3,4-b]pyridin-4 (7H)-one;
6-(2,6-Difluorophenyl)-1-(2-((2R,6S)-2,6-dimethylmorpholino)ethyl)-3-ethyl-5-hydroxy-1H-pyrazolo[3,4-b]pyridin-4(7H)-one;
1-Cyclopropyl-6-(2,6-difluorophenyl)-5-hydroxy-3-(morpholinomethyl)-1H-pyrazolo[3,4-b]pyridin-4(7H)-one;
6-(2-Chlorophenyl)-1-(2-((2R,6S)-2,6-dimethylmorpholino)ethyl)-3-ethyl-5-hydroxy-1H-pyrazolo[3,4-b]pyridin-4(7H)-one;
6-(2,6-Difluorophenyl)-3-ethyl-5-hydroxy-1-(2-hydroxyethyl)-1H-pyrazolo[3,4-b]pyridin-4(7H)-one;
6-(2,6-Difluorophenyl)-1-(4-fluorophenyl)-5-hydroxy-3-(tetrahydro-2H-pyran-4-yl)-1H-pyrazolo[3,4-b]pyridin-4(7H)-one;
6-(2,6-Difluorophenyl)-1-(2-(4,4-difluoropiperidin-1-yl)ethyl)-3-ethyl-5-hydroxy-1H-pyrazolo[3,4-b]pyridin-4(7H)-one;
6-(2,6-Difluorophenyl)-1-(3-((2R,6S)-2,6-dimethylmorpholino)propyl)-3-ethyl-5-hydroxy-1H-pyrazolo[3,4-b]pyridin-4(7H)-one;
6-(2,6-Difluorophenyl)-3-ethyl-5-hydroxy-1-((tetrahydro-2H-pyran-4-yl)methyl)-1H-pyrazolo[3,4-b]pyridin-4(7H)-one;
6-(2-Chlorophenyl)-3-ethyl-5-hydroxy-1-((tetrahydro-2H-pyran-4-yl)methyl)-1H-pyrazolo[3,4-b]pyridin-4(7H)-one;
6-(2,6-Difluorophenyl)-5-hydroxy-1-methyl-3-((tetrahydro-2H-pyran-4-yl)methyl)-1H-pyrazolo[3,4-b]pyridin-4(7H)-one;
6-(2-Chlorophenyl)-1-(2-(4,4-difluoropiperidin-1-yl)ethyl)-3-ethyl-5-hydroxy-1H-pyrazolo[3,4-b]pyridin-4(7H)-one;
6-(2,6-Difluorophenyl)-3-ethyl-5-fluoro-1-(2-(tetrahydro-2H-pyran-4-yl)ethyl)-1H-pyrazolo[3,4-b]pyridin-4(7H)-one;
6-(2-Chlorophenyl)-3-ethyl-5-hydroxy-1-(2-(tetrahydro-2H-pyran-4-yl)ethyl)-1H-pyrazolo[3,4-b]pyridin-4(7H)-one;
3-Ethyl-6-(2-fluoro-3-(trifluoromethyl)phenyl)-5-hydroxy-1-(2-(tetrahydro-2H-pyran-4-yl)ethyl)-1H-pyrazolo[3,4-b]pyridin-4(7H)-one;
6-(2,6-Difluorophenyl)-5-hydroxy-3-((4-isobutylpiperazin-1-yl)methyl)-1-methyl-1H-pyrazolo[3,4-b]pyridin-4(7H)-one;
6-(2,6-difluorophenyl)-5-hydroxy-3-(hydroxymethyl)-1-((tetrahydro-2H-pyran-4-yl)methyl)-1H-pyrazolo[3,4-b]pyridin-4(7H)-one;
3-((4-Cyclopropylpiperazin-1-yl)methyl)-6-(2,6-difluorophenyl)-5-hydroxy-1-methyl-1H-pyrazolo[3,4-b]pyridin-4(7H)-one;
6-(2,6-difluorophenyl)-5-hydroxy-3-(hydroxymethyl)-1-(2-(tetrahydro-2H-pyran-4-yl)ethyl)-1H-pyrazolo[3,4-b]pyridin-4(7H)-one;
6-(2,6-Difluorophenyl)-3-ethyl-5-hydroxy-1-(2-(methylsulfonyl)ethyl)-1H-pyrazolo[3,4-b]pyridin-4(7H)-one;
6-(2,6-Difluorophenyl)-5-hydroxy-1-methyl-3-((4-(oxetan-3-yl)piperazin-1-yl)methyl)-1H-pyrazolo[3,4-b]pyridin-4(7H)-one;
6-(2,6-Difluorophenyl)-5-hydroxy-1-methyl-3-((4-(2,2,2-trifluoroethyl)piperazin-1-yl)methyl)-1H-pyrazolo[3,4-b]pyridin-4(7H)-one;
6-(2,6-Difluorophenyl)-3-((4-(2-fluoroethyl)piperazin--yl)methyl)-5-hydroxy-1-methyl-1H-pyrazolo[3,4-b]pyridin-4(7H)-one;
(S)-6-(2,6-Difluorophenyl)-5-hydroxy-3-((4-isopropyl-3-methylpiperazin-1-yl)methyl)-1-methyl-1H-pyrazolo[3,4-b]pyridin-4(7H)-one;
(R)-6-(2,6-Difluorophenyl)-5-hydroxy-3-((4-isopropyl-3-methylpiperazin-1-yl)methyl)-1-methyl-1H-pyrazolo[3,4-b]pyridin-4(7H)-one;
6-(2,6-Difluorophenyl)-5-hydroxy-1-methyl-3-((3-(trifluoromethyl)-5,6-dihydro-[ 1,2,4] triazolo[4,3-a]pyrazin-7(8H)-yl)methyl)-1H-pyrazolo[3,4-b]pyridin-4(7H)-one;
5-hydroxy-6-(4-hydroxyphenyl)-1-methyl-3-(trifluoromethyl)-1,7-dihydro-4H-pyrazolo[3,4-b]pyridin-4-one;
6-(2,6-difluorophenyl)-1,3-dimethyl-4-oxo-4,7-dihydro-1H-pyrazolo[3,4-b]pyridin-5-yl methyl carbonate;
5-(2-chlorophenyl)-6-hydroxy-2,3-dimethylpyrano[3,2-c]pyrazol-7(2H)-one;
5-(2-Chlorophenyl)-6-hydroxy-3-methyl-2-(propan-2-yl)pyrano[3,2-c]pyrazol-7(2H)-one;
5-(2-Chlorophenyl)-2-ethyl-6-hydroxy-3-methylpyrano[3,2-c]pyrazol-7(2H)-one;
6-(2-chlorophenyl)-5-hydroxy-1,3-dimethylpyrano[2,3-c]pyrazol-4(1H)-one;
6-(2-Chlorophenyl)-1-(4-fluorophenyl)-5-hydroxy-3-methylpyrano[2,3-c]pyrazol-4(1H)-one;
6-(2,6-Difluorophenyl)-5-hydroxy-1-(3-hydroxypropyl)-3-methyl-1H-pyrazolo[3,4-b]pyridin-4(7H)-one;
6-(2-chlorophenyl)-5-methoxy-1,3-dimethyl-1H-pyrazolo[3,4-b]pyridin-4(7H)-one;
6-(2,6-Difluorophenyl)-1-ethyl-5-methoxy-3-methyl-1H-pyrazolo[3,4-b]pyridin-4(7H)-one;
N-[4-(1-ethyl-5-hydroxy-3-methyl-4-oxo-4,7-dihydro-1H-pyrazolo[3,4-b]pyridin-6-yl)-3-fluorophenyl]methanesulfonamide;
N-[4-(1-Ethyl-5-hydroxy-3-methyl-4-oxo-4,7-dihydro-1H-pyrazolo[3,4-b]pyridin-6-yl)-3,5-difluorophenyl]methanesulfonamide;
N-{3-Fluoro-4-[5-hydroxy-1-methyl-4-oxo-3-(trifluoromethyl)-4,7-dihydro-1H-pyrazolo[3,4-b]pyridin-6-yl]phenyl}methanesulfonamide;
6-(2-Chlorophenyl)-3-ethyl-5-hydroxy-1-(2-morpholinoethyl)-1H-pyrazolo[3,4-b]pyridin-4(7H)-one hydrochloride;
6-(2-Fluoro-3-(trifluoromethyl)phenyl)-5-hydroxy-3-methyl-1-(2-morpholinoethyl)-1H-pyrazolo[3,4-b]pyridin-4(7H)-one dihydrochloride;
6-(2-Chlorophenyl)-5-hydroxy-3-methyl-1-(2-(4-methylpiperazin-1-yl)ethyl)-1H-pyrazolo[3,4-b]pyridin-4(7H)-one trihydrochloride;
6-(2,6-Difluorophenyl)-3-ethyl-5-hydroxy-1-(2-(4-methylpiperazin-1-yl)ethyl)-1H-pyrazolo[3,4-b]pyridin-4(7H)-one trihydrochloride;
6-(2,6-Difluorophenyl)-3-ethyl-5-hydroxy-1-(2-morpholinoethyl)-1H-pyrazolo[3,4-b]pyridin-4(7H)-one dihydrochloride;
6-(2-Chlorophenyl)-5-hydroxy-3-methyl-1-(2-(4-methylpiperazin-1-yl)ethyl)-1H-pyrazolo[3,4-b]pyridin-4(7H)-one fumarate;
6-(2,6-Difluorophenyl)-3-ethyl-5-hydroxy-1-(2-(4-methylpiperazin-1-yl)ethyl)-1H-pyrazolo[3,4-b]pyridin-4(7H)-one fumarate;
6-(2-Chlorophenyl)-3-ethyl-5-hydroxy-1-(2-(4-methylpiperazin-1-yl)ethyl)-1H-pyrazolo[3,4-b]pyridin-4(7H)-one fumarate;
6-(2-Chlorophenyl)-5-hydroxy-3-methyl-1-(2-(4-methylpiperazin-1-yl)ethyl)-1H-pyrazolo[3,4-b]pyridin-4(7H)-one hemi fumarate;
6-(2-Fluoro-3-(trifluoromethyl)phenyl)-5-hydroxy-3-methyl-1-(2-morpholinoethyl)-1H-pyrazolo[3,4-b]pyridin-4(7H)-one hemi fumarate;
6-(2-Chlorophenyl)-3-ethyl-5-hydroxy-1-(2-(4-methylpiperazin-1-yl)ethyl)-1H-pyrazolo[3,4-b]pyridin-4(7H)-one hemi fumarate;
6-(2-Chlorophenyl)-5-hydroxy-3-methyl-1-(2-(4-methylpiperazin-1-yl)ethyl)-1H-pyrazolo[3,4-b]pyridin-4(7H)-one trimethanesulfonate;
6-(2,6-Difluorophenyl)-3-ethyl-5-hydroxy-1-(2-(4-methylpiperazin-1-yl)ethyl)-1H-pyrazolo[3,4-b]pyridin-4(7H)-one trimethanesulfonate;
3-Ethyl-6-(2-fluoro-3-(trifluoromethyl)phenyl)-5-hydroxy-1-(2-morpholinoethyl)-1H-pyrazolo[3,4-b]pyridin-4(7H)-one trimethanesulfonate;
6-(2-Chlorophenyl)-5-hydroxy-3-methyl-1-(2-(4-methylpiperazin-1-yl)ethyl)-1H-pyrazolo[3,4-b]pyridin-4(7H)-one hemi 2-hydroxypropane-1,2,3-tricarboxylate;
6-(2-Chlorophenyl)-3-ethyl-5-hydroxy-1-(2-(4-methylpiperazin-1-yl)ethyl)-1H-pyrazolo[3,4-b]pyridin-4(7H)-one hemi 2-hydroxypropane-1,2,3-tricarboxylate;
6-(2,6-Difluorophenyl)-3-ethyl-5-hydroxy-1-(2-(4-methylpiperazin-1-yl)ethyl)-1H-pyrazolo[3,4-b]pyridin-4(7H)-one hemi 2-hydroxypropane-1,2,3-tricarboxylate;
6-(2-Chlorophenyl)-5-hydroxy-3-methyl-1-(2-(4-methylpiperazin-1-yl)ethyl)-1H-pyrazolo[3,4-b]pyridin-4(7H)-one 2-hydroxypropane-1,2,3-tricarboxylate;
6-(2,6-difluorophenyl)-3-ethyl-5-hydroxy-1-(2-(4-methylpiperazin-1-yl)ethyl)-1H-pyrazolo[3,4-b]pyridin-4(7H)-one 2-hydroxypropane-1,2,3-tricarboxylate;
6-(2-Chlorophenyl)-3-ethyl-5-hydroxy-1-(2-(4-methylpiperazin-1-yl)ethyl)-1H-pyrazolo[3,4-b]pyridin-4(7H)-one 2-hydroxypropane-1,2,3-tricarboxylate;
and pharmaceutically acceptable salts thereof.

39. The compound according to claim 38, wherein the compound has the formula 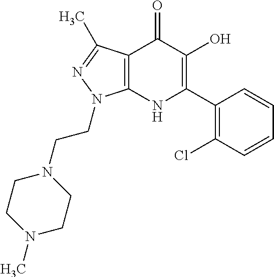 [SMILES: Cc1nn(CCN2CCN(C)CC2)c2[nH]c(-c3ccccc3Cl)c(O)c(=O)c12]
or a pharmaceutically acceptable salt thereof.

40. The compound according to claim 38, wherein the compound has the formula 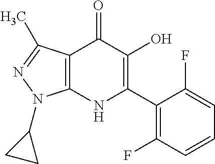 [SMILES: Cc1nn(C2CC2)c2[nH]c(-c3c(F)cccc3F)c(O)c(=O)c12]
or a pharmaceutically acceptable salt thereof.

41. The compound according to claim 38, wherein the compound has the formula 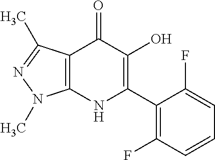 [SMILES: Cc1nn(C)c2[nH]c(-c3c(F)cccc3F)c(O)c(=O)c12]
or a pharmaceutically acceptable salt thereof.

42. The compound according to claim 38, wherein the compound has the formula 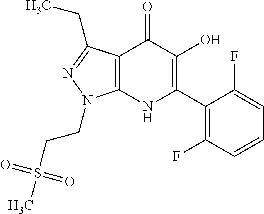 [SMILES: CCc1nn(CCS(C)(=O)=O)c2[nH]c(-c3c(F)cccc3F)c(O)c(=O)c12]
or a pharmaceutically acceptable salt thereof.

43. A pharmaceutical composition comprising a compound according to claim 38 and a pharmaceutically acceptable excipient.

44. The pharmaceutical composition according to claim 43, wherein the pharmaceutically acceptable excipient is a carrier or diluent.

45. A method of treating a NADPH oxidase mediated disease, disorder, syndrome, or condition in a subject comprising administering an effective amount of a compound according to claim 38.

46. The method according to claim 45, wherein the disease, disorder, syndrome or condition is pain, diabetes, cystic fibrosis osteoporosis, asthma, cough, chronic obstructive pulmonary diseases, COPD exacerbation, non-small cell lung cancer, breast cancer, prostate cancer, non-alcoholic fatty liver disease, non-alcoholic steatohepatitis, primary biliary cirrhosis or cirrhosis.

47. The method according to claim 45, wherein the disease, disorder, syndrome or condition is non-alcoholic fatty liver disease, non-alcoholic steatohepatitis, primary cirrhosis or cirrhosis.
